(19) United States
(12) Patent Application Publication
(10) Pub. No.: US 2026/0297085 A1
(43) Pub. Date: Oct. 1, 2026

(54) 1 H-PYRROLO[2,3-B]PYRIDIN-4-YL]-2-OXOPYRROLIDINE-3-CARBONITRILE DERIVATIVES AS TYROSINE KINASE 2 (TYK2) INHIBITORS FOR THE TREATMENT OF INFLAMMATORY DISEASES

(71) Applicant: BIOGEN MA INC., Cambridge, MA (US)

(72) Inventors: Nathan Edward Genung, Charlestown, MA (US); Tamara Levin, Belmont, MA (US)

(21) Appl. No.: 19/472,217

(22) PCT Filed: Apr. 5, 2024
(86) PCT No.: PCT/US2024/023255

§ 371 Date: Oct. 3, 2025

Related U.S. Application Data
(60) Provisional application No. 63/457,857, filed on Apr. 7, 2023.

(51) Int. Cl. C07D 471/04; A61K 31/428; A61K 31/4355; A61K 31/437; A61K 31/444; A61K 31/5025; C07D 417/14; C07D 487/04; C07D 491/048; C07D 513/04

(52) U.S. Cl. CPC C07D 471/04; A61K 31/428; A61K 31/4355; A61K 31/437; A61K 31/444; A61K 31/5025; C07D 417/14; C07D 487/04; C07D 491/048; C07D 513/04

(57) ABSTRACT

This disclosure relates to compounds of Formula (I-1) or (I-2): The compounds of the present disclosure are capable of inhibiting the activity of tyrosine kinase 2 (TYK2) and are useful for the treatment of diseases or disorders, such as e.g. inflammation, autoimmune disease, neuroinflammation, arthritis, rheumatoid arthritis, spondyloarthropathies, systemic lupus erythematous, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis and sunburn. The disclosure further provides methods of preparing the compounds.

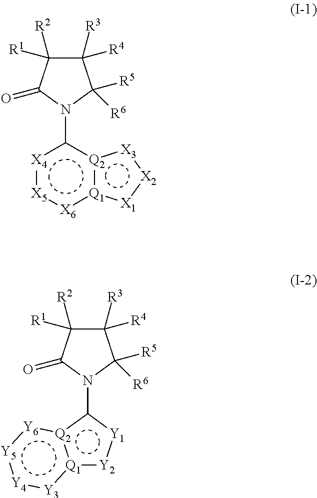

### RELATED APPLICATIONS

This application claims priority to U.S. Provisional Application No. 63/457,857, filed on Apr. 7, 2023. The entire contents of the foregoing application are expressly incorporated herein by reference.

### FIELD OF THE INVENTION

The present disclosure relates to inhibitors of Tyrosine kinase 2 (TYK2), and pharmaceutically acceptable salts 5 thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering the TYK2 inhibitor to a warm-blooded animal, especially a 10 human.

### BACKGROUND OF THE INVENTION

Cytokines are small secreted proteins released by cells and have a specific effect on the interactions and communications between cells. Cytokine pathways mediate a broad range of biological functions including many aspects of inflammation and immunity through mostly extracellular signaling.

Tyrosine kinase 2 (TYK2) is a member of Janus kinases (JAK) that are cytoplasmic protein kinases associated with cytokine receptors and play a central role in mediating cytokine signaling (Kisseleva et al., Gene, 2002, 285, 1; and Yamaoka et al. Genome Biology 2004, 5, 253). The JAK family also includes JAK1, JAK2 and JAK3. More specifically, cytokine's engagement with cognate receptors triggers activation of receptors associate with JAK, which leads to JAK mediated tyrosine phosphorylation of signal transducer and activator of transcription (STAT) proteins and ultimately transcriptional activation of specific gene sets (Schindler et al, 2007, J. Biol. Chem. 282: 20059-63). Numerous cytokines known to activate the JAK family include the interferon (IFN) family (IFN-alpha, IFN-beta, IFN-omega, Limitin, IFN-gamma, IL-10, IL-19, IL-20, IL-22), the glycoprotein (gp) 130 family (IL-6, IL-11, OSM, LlF, CNTF, NNT-1/BSF-3, G-CSF, CT-1, Leptin, IL-12, IL-23), the gamma C family (IL-2, IL-7, TSLP, IL-9, IL-15, IL-21, IL-4, IL-13), IL-3 family (IL-3, IL-5, GM-CSF), the single chain family (EPO, GH, PRL, TPO), receptor tyrosine kinases (EGF, PDGF, CSF-1, HGF), and G-protein coupled receptors (AT1).

TYK2 is important in the signaling of the type I interferons (e.g., IFN-alpha), IL-6, IL-10, IL-12 and IL-23 (Liang, Y. et al., Expert Opinion on Therapeutic Targets, 2014, 18, 5, 571-580; Kisseleva et al., 2002, Gene 285:1-24; and Watford, W. T. & O'Shea, J. J., 2006, Immunity 25:695-697). Consistent with this, primary cells derived from a TYK2 deficient human are defective in type I interferon, IL-6, IL-10, IL-12 and IL-23 signaling. TYK2 signals with other members of the JAK family in the following combinations: TYK2/JAK1, TYK2/JAK2, TYK2/JAK1/JAK2.

Studies have shown that inappropriate JAK activities can arise from mutation, over-expression, or inappropriate regulation, dys-regulation or de-regulation, as well as over- or under-production of growth factors or cytokines, and therefore trigger a variety of biological cellular responses relating to cell growth, cell differentiation, cell function, survival, apoptosis, and cell mobility. The inappropriate JAK activities are implicated in many diseases that include but not limited to cancer, cardiovascular diseases, allergies, asthma and other respiratory diseases, autoimmune diseases, inflammatory diseases, bone diseases, metabolic disorders, and neurological and neurodegenerative disorders such as Alzheimer's disease.

Small molecule JAK inhibitors have emerged as a major therapeutic advancement in treating autoimmune diseases. To date, all known small molecule JAK inhibitors that have progressed into development are active site-directed inhibitors that bind to the adenosine triphosphate (ATP) site of the catalytic domain (also referred to as the JH1 or “Janus Homology 1” domain) of the JAK protein, which prevents catalytic activity of the kinase by blocking ATP, downstream phosphorylation, and resulting pathway signal transduction (Bryan et al., J. Med. Chem. 2018, 61, 9030-9058).

Because of the high homology of the ATP active site across the kinome and especially within the JAK family, it is a significant challenge to achieve high selectivity for a specific JAK family member while also maintaining selectivity within the kinome. As a result, many JAK inhibitors that have been developed are pan-JAK inhibitors or are modestly selective for one or more JAK family members. While these inhibitors have shown encouraging results in treating autoimmune diseases, undesirable side effects leading to a narrow therapeutic index have been observed and suggest the need for improved treatments.

TYK2 has been shown to be important in the differentiation and function of multiple cell types important in inflammatory disease and autoimmune disease including natural killer cells, B cells, and T helper cell types. Aberrant TYK2 expression is associated with multiple autoimmune or inflammatory conditions.

There remains a need for potent compounds that demonstrate high selectivity for TYK2 over other members of the JAK family.

### SUMMARY OF THE INVENTION

One aspect of the present disclosure are compounds of Formula (I-1) or (I-2):

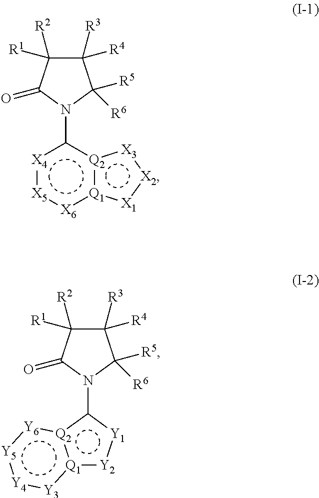

or a pharmaceutically acceptable salt thereof, wherein:
Q₁ and Q₂ each independently represent C or N;
X₁ is N, N—R^{X1}, or C—R^{X1};
X₂ is S, N—R^{X2} or C—R^{X2};
X₃ is N, O, S or C—R^{X3}, provided when X₂ is 5, then X₃ cannot be S or O;
X₄ is N or C—R^{X4};
X₅ is C—R^{X5};
X₂ is N or C—R^{X6};
Y₁ is N or C—R^{Y1};
Y₂ is N—R² or C—R^{Y2};
Y₃ is N or C—R^{X6};
Y₄ is N or C—R^{Y4};
Y₅ is C—R^{Y5};
Y₆ is C—R^{Y6};
R^{X1}, R^{X3}, R^{X4} and R^{X6} are each independently selected from H, halo, —CN, —NR^{1a}R^{1b}, —OR^{1c}, C₁₋₄ alkyl and C₁₋₄ haloalkyl;
R^{X2} and R^{X5} are each independently selected from H, halo, CN, —NR^{1a}R^{1b}, —OR^{1c}, C₁₋₆ alkyl, C₃₋₈ cycloalkyl and R^{S}, wherein C₁₋₆ alkyl and C₃₋₈ cycloalkyl represented by R^{X2} and R^{X5} are each optionally substituted with one or more R⁸;
R^{Y1}, R^{Y3}, R^{Y4} and R^{Y6} are each independently selected from H, halo, —CN, —NR^{1a}R^{1b}, —OR^{1c}, C₁₋₄ alkyl and C₁₋₄ haloalkyl;
R^{Y2} and R^{Y5} are each independently selected from H, halo, —CN, —NR^{1a}R^{1b}, —OR^{1c}, C₁₋₆ alkyl, C₃₋₈ cycloalkyl and R^{S}, wherein C₁₋₆ alkyl and C₃₋₈ cycloalkyl represented by R^{Y2} and R^{Y5} are each optionally substituted with one or more R⁸;
R^{S} is independently selected from C₆₋₁₀ aryl, 4 to 7 membered monocyclic heterocyclyl and 5 to 10 membered heteroaryl, wherein the C₆₋₁₀ aryl, 4 to 7 membered monocyclic heterocyclyl and 5 to 10 membered heteroaryl represented by R^{S} are each optionally substituted with one or more R⁷;
R¹, R², R³, R⁴, R⁵ and R⁶, are each independently selected from H, halo, —CN, —NR^{1a}R^{1b}, —OR^{1c}, C₁₋₆ alkyl, C₃₋₈ cycloalkyl, C₆₋₁₀ aryl, 4 to 10 membered heterocycloalkyl and 5 to 10 membered heteroaryl, wherein the C₁₋₆ alkyl, C₃₋₈ cycloalkyl, C₆₋₁₀ aryl, 4 to 10 membered heterocycloalkyl and 5 to 10 membered heteroaryl represented by R¹, R², R³, R⁴, R⁵ and R⁶ are each optionally substituted with one or more substituents independently selected from halo, —CN, —NR^{1a}R^{1b}, —OR^{1c}, C₁₋₆ alkyl and C₃₋₈ cycloalkyl;
R⁷ is independently selected from halo, —CN, —NR^{1a}R^{1b}, —NR^{1a}C(O)R^{1d}, —OR^{1c}, —C(O)OR^{1c}, —C(O)NR^{1a}R^{1b}, —SO₂R^{1c}, C₁₋₆ alkyl, C₁₋₆ haloalkyl, C₃₋₆ cycloalkyl, C₆₋₁₀ aryl, 4 to 7 membered monocyclic heterocycloalkyl, 7 to 10 membered bicyclic heterocycloalkyl, and 5 to 6 membered heteroaryl; wherein the C₁₋₆ alkyl, C₁₋₆ haloalkyl, C₃₋₆ cycloalkyl, C₆₋₁₀ aryl, 4 to 7 membered monocyclic heterocycloalkyl, 7 to 10 membered bicyclic heterocycloalkyl, and 5 to 6 membered heteroaryl represented by R⁷ are each optionally substituted with one or more R^{7a};
or alternatively two of R⁷, taken together with their intervening atoms, for a 3- to 7-membered monocyclic heterocyclyl that is optionally substituted by one or two substituents independently selected from C₁₋₆ alkyl and oxo (═O);
R^{7a}, for each occurrence, is independently selected from halo, —CN, C₁₋₄ alkyl, C₁₋₄ haloalkyl, C₃₋₆ cycloalkyl, —NR^{1a}R^{1b}, —OR^{1c} and 4 to 6 membered monocyclic heterocycloalkyl, wherein the C₁₋₄ alkyl, C₃₋₆ cycloalkyl and 4 to 6 membered monocyclic heterocycloalkyl are each optionally substituted with one or more substituents independently selected from C₁₋₃alkyl, —OR^{1c}, CN and halo;
R⁸, for each occurrence, is independently halo, —CN, —NR^{1a}R^{1b}, —OR^{1c}, —C(O)OR^{1c}, C₁₋₆ alkyl, C₁₋₆ haloalkyl, C₃₋₆ cycloalkyl, C₆₋₁₀ aryl, 4 to 7 membered monocyclic heterocycloalkyl, or 5 to 6 membered heteroaryl; wherein the C₁₋₆ alkyl, C₁₋₆ haloalkyl, C₃₋₆ cycloalkyl, C₆₋₁₀ aryl, 4 to 7 membered monocyclic heterocycloalkyl, and 5 to 6 membered heteroaryl represented by R¹ are each optionally substituted with one or more substituents independently selected from halo, C₁₋₄ alkyl, C₁₋₄haloalkyl, C₃₋₆ cycloalkyl, —NR^{1a}R^{1b}, —OR^{1c}; and 4 to 6 membered monocyclic heterocycloalkyl;
R^{1a} and R^{1b} are each independently H or C₁₋₄ alkyl;
R^{1c} is H, C₁₋₄ alkyl or C₁₋₄ haloalkyl;
R^{1d} is C₁₋₄ alkyl or —OR^{1e};
R^{1e} is C₁₋₄ alkyl, and
wherein at least one of R^{X1}, R^{X2}, R^{X3}, R^{X4}, R^{X5} and R^{X6} is not H, and at least one of R^{Y1}, R^{Y2}, R^{Y3}, R^{Y4}, R^{Y5} and R^{Y6} is not H.

In one aspect, the present disclosure is a pharmaceutical composition comprising at least one compound described herein, or a pharmaceutically acceptable salt thereof, and at least one pharmaceutically acceptable carrier.

Another aspect of the present disclosure is a method of inhibiting TYK2 activity in a subject in need thereof comprising administering to the subject an effective amount of at least one compound described herein, or a pharmaceutically acceptable salt thereof, or a pharmaceutical composition described herein.

In some aspect, the present disclosure is a method of treating a disease or disorder responsive to inhibition of TYK2 in a subject comprising administering to the subject an effective amount of at least one compound described herein, or a pharmaceutically acceptable salt thereof, or a pharmaceutical composition described herein.

The present disclosure also includes the use of at least one compound described herein, or a pharmaceutically acceptable salt thereof, or a pharmaceutically composition described herein for the manufacture of a medicament for inhibiting TYK2 activity. Also included is the use of at least one compound described herein, or a pharmaceutically acceptable salt thereof, or a pharmaceutically composition described herein for the manufacture of a medicament for treating a disease or disorder responsive to inhibition of TYK2.

The disclosure also provides a compound described herein, or a pharmaceutically acceptable salt thereof, or a pharmaceutical composition described herein for use in inhibiting TYK2 activity. Also provided is a compound described herein, or a pharmaceutically acceptable salt thereof, or a pharmaceutical composition described herein for use in treating a disease or disorder responsive to inhibition of TYK2.

Other features or advantages will be apparent from the following detailed description of several embodiments, and also from the appended claims.

### DETAILED DESCRIPTION OF THE INVENTION

The compounds or pharmaceutically acceptable salts thereof described herein demonstrate high potency against TYK2. In addition, the compounds or pharmaceutically acceptable salts thereof of the present disclosure have high selectivity for inhibiting TYK2 over other members of JAK family, such as JAK1, JAK2 and JAK3.

### Compounds and Compositions

In a first embodiment, the present disclosure provides a compound of Formula (I-1) or (I-2):

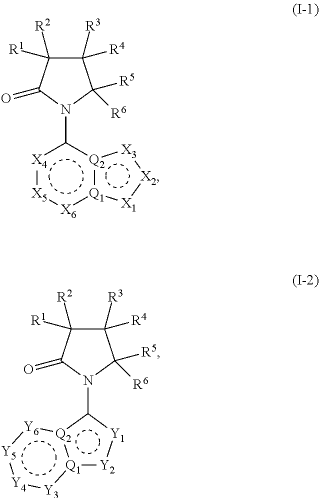

or a pharmaceutically acceptable salt thereof, wherein the variables in Formula (I-1) or (I-2) are as described in the first embodiment.

In a second embodiment, for the compounds of Formula (I-1) or (I-2), or a pharmaceutically acceptable salt thereof:
(i) Q₁ is C, and Q₂ is C;
(ii) Q₁ is N, and Q₂ is C; or
(iii) Q₁ is C, and Q₂ is N; and the remaining variables are as described in the first embodiment.

In a third embodiment, for the compounds of Formula (I-1), or a pharmaceutically acceptable salt thereof:
(i) X₁ is C—R^{X1}, X₂ is C—R^{X2}; X₄ is N or C—R^{X4}, X₅ is C—R^{X5}, X₆ is N, and X₃ is selected from N, S, O and C—R^{X3}; or
(ii) X₁ is N, X₂ is C—R^{X2}, X₄ is N or C—R^{X4}, X₅ is C—R^{X5}, X₆ is C—R^{Y6}, and X₃ is selected from N, S and C—R^{X3}; and the remaining variables are as described in the first or second embodiment.

In a fourth embodiment, for the compounds of Formula (I-1), or a pharmaceutically acceptable salt thereof, one of R^{X2} and R^{X5} is R^{S}, and the other one is H; and the remaining variables are as described in the third embodiment.

In a fifth embodiment, for the compounds of Formula (I-2), or a pharmaceutically acceptable salt thereof:
(i) Y₁ is N, Y₂ is N—R^{Y2}, Y₃ is N, Y₄ is C—R^{Y4}, Y₅ is C—R^{Y5}, and Y₆ is C—R^{Y6}; (ii) Y₁ is C—R^{Y1}, Y₂ is N—R^{Y2}, Y₃ is N, Y₄ is C—R^{Y4}, Y₅ is C—R^{Y5}, and Y₆ is C—R^{Y6}; (iii) Y₁ is C—R^{Y1}, Y₂ is C—R^{Y2}, Y₃ is N, Y₄ is C—R^{Y4}, Y₅ is C—R^{Y5}, and Y₆ is C—R^{Y6}; or
(iv) Y₁ is N, Y₂ is N—R^{Y2}, Y₃ is C—R^{Y3}, Y₄ is N, Y₅ is C—R^{Y5}, and Y₆ is C—R^{Y6}; and the remaining variables are as described in the first or second embodiment.

In a sixth embodiment, for the compounds of Formula (I-2), or a pharmaceutically acceptable salt thereof, one of R^{Y2} and R^{Y5} is R^{S} and the other one is H; and the remaining variables are as described in the fifth embodiment.

In a seventh embodiment, the compound of the present disclosure is represented by one of the following formulae:

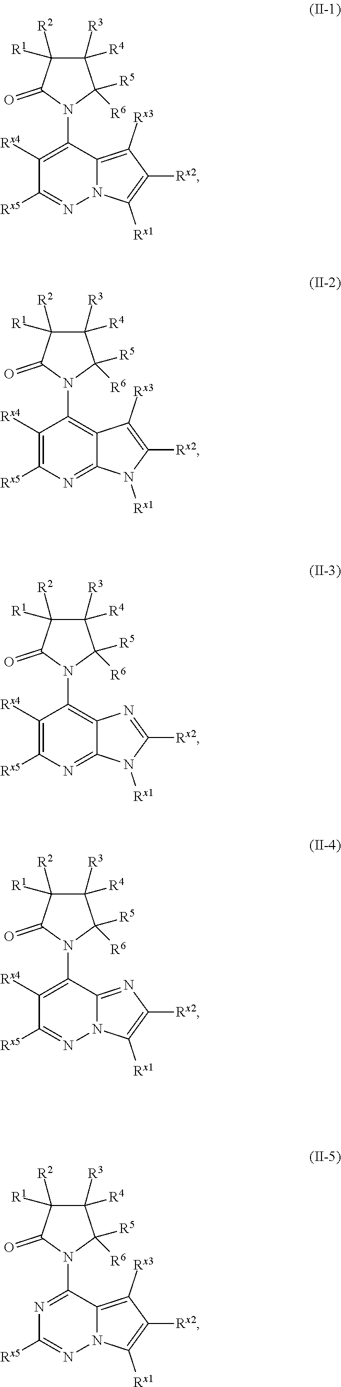

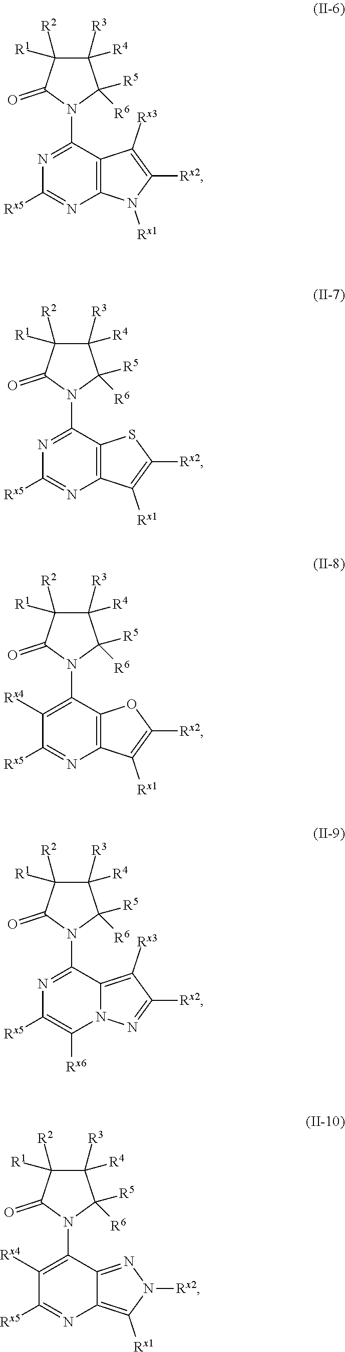

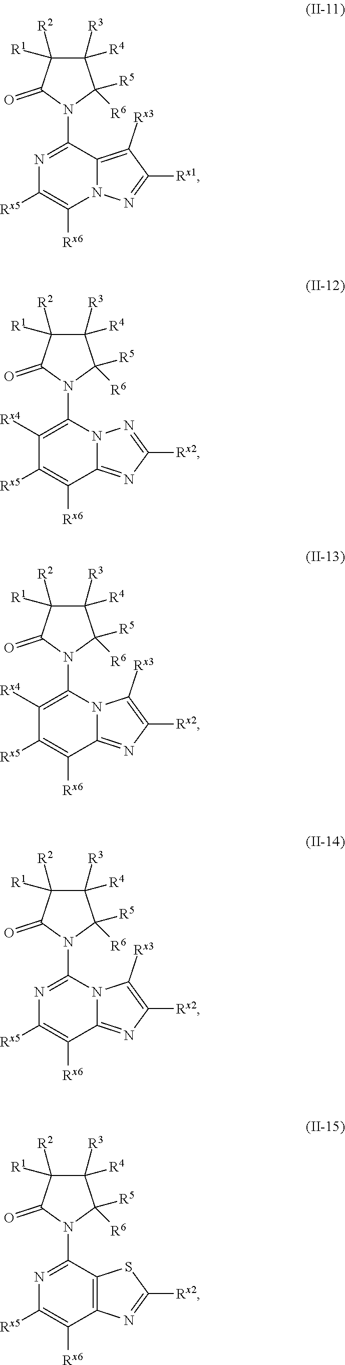

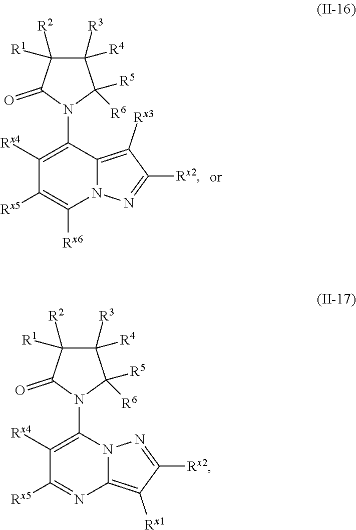

or a pharmaceutically acceptable salt thereof, wherein the variables in Formula (II-1), (II-2), (II-3), (II-4), (II-5), (II-6), (II-7), (II-8), (II-9), (II-10), (II-11), (II-12), (II-13), (II-14), (II-15), (II-16), and (II-17) are as described in the first embodiment.

In an eight embodiment, for the compounds of Formula (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), or (II-17), or a pharmaceutically acceptable salt thereof, R^{X2} is 4- to 7-membered monocyclic heterocyclyl or 5- to 10-membered heteroaryl, wherein the 4- to 7-membered monocyclic heterocyclyl and 5- to 10-membered heteroaryl represented by R^{X2} are each optionally substituted with 1 or 2 R⁷; and R^{X5}, if present, is H; and the reminding variables are as described in the first, second, third, fourth, or seventh embodiment.

In a ninth embodiment, for the compounds of Formula (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), or (II-17), or a pharmaceutically acceptable salt thereof, R^{X5} is 4- to 7-membered monocyclic heterocyclyl or 5 to 10 membered heteroaryl, wherein the 4- to 7-membered monocyclic heterocyclyl and 5 to 10 membered heteroaryl represented by R^{X5} are each optionally substituted with 1 or 2 R⁷; and R^{X2}, if present, is H; and the reminding variables are as described in the first, second, third, fourth, or seventh embodiment above.

In a tenth embodiment, the compound of the present disclosure is represented by one of the following formula:

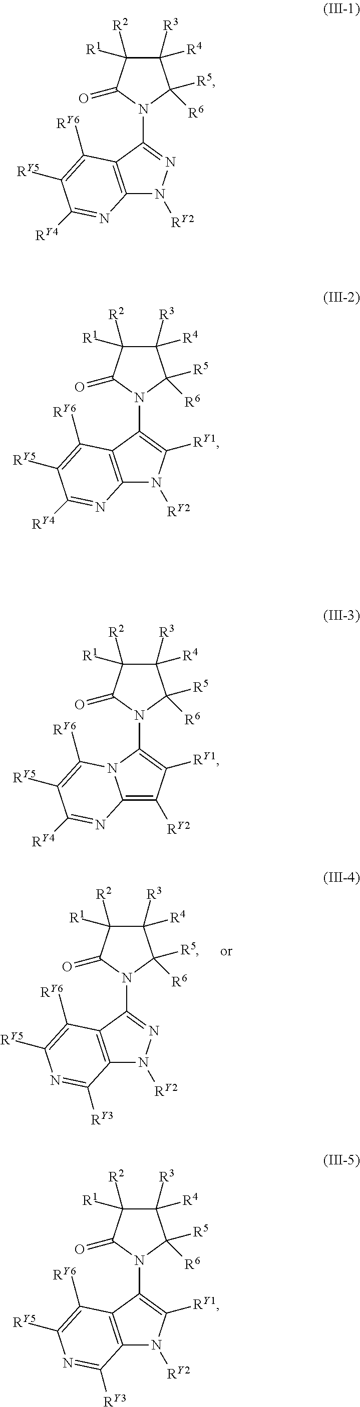

or a pharmaceutically acceptable salt thereof, wherein the variables in Formula (III-1), (III-2), (III-3), (III-4), and (III-5) are as described in the first embodiment.

In an eleventh embodiment, for the compounds of Formula (I-1), (I-2), (III-1), (III-2), (III-3), (III-4), or (III-5), or a pharmaceutically acceptable salt thereof, R^{Y2} is R^{S}; and R^{S} is 4-to 7-membered monocyclic heterocyclyl or 5- to 10-membered heteroaryl, wherein the 4- to 7-membered monocyclic heterocyclyl and 5- to 10-membered heteroaryl represented by R^{S} are each optionally substituted with 1 or 2 R⁷; and R^{Y5} is H; and the remaining variables are as described in the first, second, fifth, sixth, or tenth embodiment.

In a twelfth embodiment, for the compounds of Formula (I-1), (I-2), (III-1), (III-2), (III-3), (III-4), or (III-5), or a pharmaceutically acceptable salt thereof, R^{Y5} is R^{S}; and R^{S} is 4-to 7-membered monocyclic heterocyclyl or 5 to 10 membered heteroaryl, wherein the 4- to 7-membered monocyclic heterocyclyl and 5 to 10 membered heteroaryl represented by R^{S} are each optionally substituted with 1 or 2 R⁷; and R^{Y2} is H; and the remaining variables are as described in the first, second, fifth, sixth, or tenth embodiment.

In a thirteenth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R^{S} is selected from pyrazine, pyridazine, pyridine, pyridin-2(1H)-one, pyrazole, pyrazolopyridine, pyrimidine, pyrrolopyridine, isoxazole, imidazole, imidazopyridine, indazole, thiazole, triazole, thiazolopyridine and triazolopyridine, each of which is optionally substituted with 1 or 2 of R⁷; and the remaining variables are as described in the eighth, ninth, eleventh, or twelfth embodiment.

In a fourteenth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R^{S} is selected from:

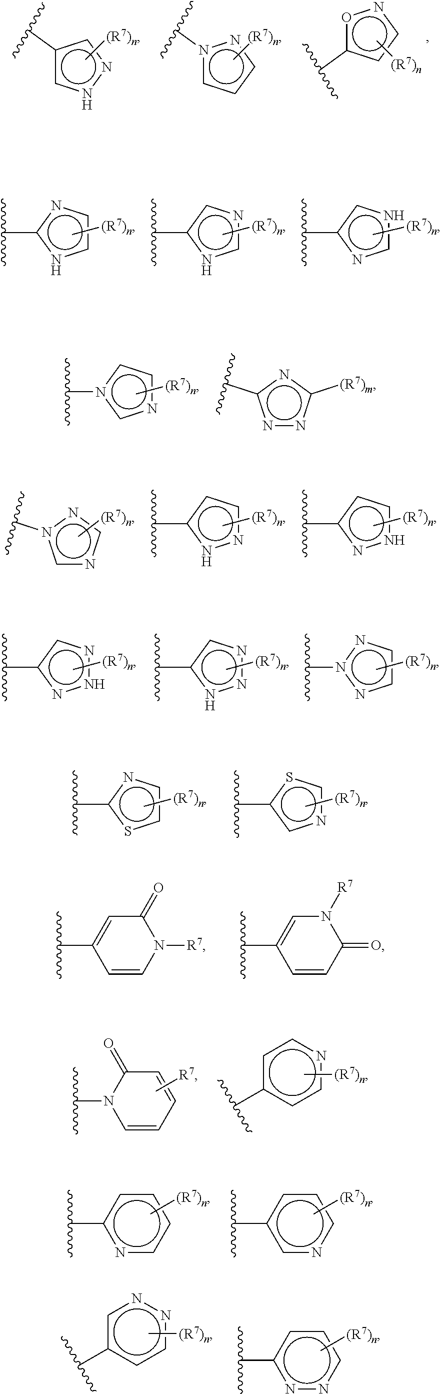

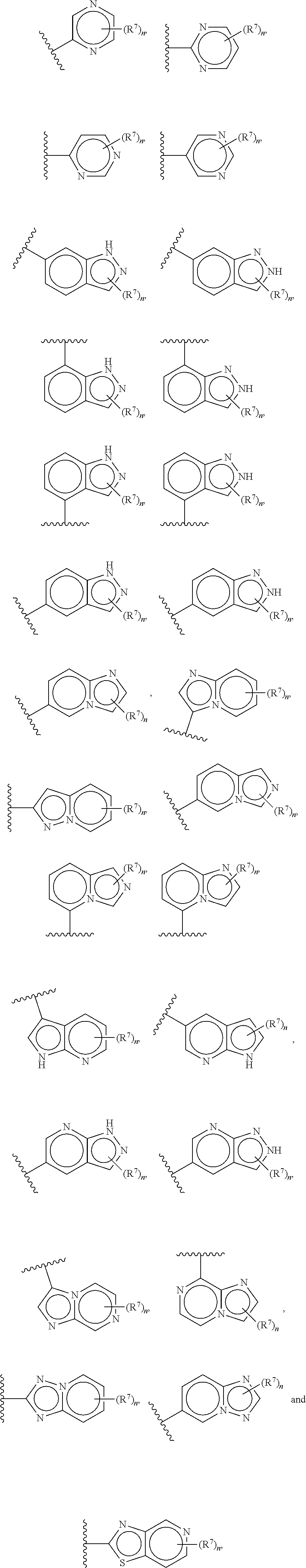

wherein

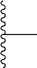

represents a bond to X₂, X₅, Y₂ or Y₅;
n represents 0, 1 or 2; and
m represents 0 or 1; and the remaining variables are as described in the eighth, ninth, eleventh, or twelfth embodiment.

In a fifteenth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof:
R⁷ is independently selected from halo, —CN, —NR^{1a}R^{1b}, —NR^{1a}C(═O)R^{1d}, —OR^{1c}, —C(O)NR^{1a}R^{1b}, —SO₂R^{1e}, C₁₋₆ alkyl, C₁₋₆ haloalkyl, C₃₋₆ cycloalkyl, 4 to 7 membered monocyclic heterocycloalkyl, 7 to 8 membered bicyclic heterocycloalkyl, and 5 to 6 membered heteroaryl; wherein the C₁₋₆ alkyl, C₁₋₆ haloalkyl, C₃₋₆ cycloalkyl, 4 to 7 membered monocyclic heterocycloalkyl, 7 to 8 membered bicyclic heterocycloalkyl, and 5 to 6 membered heteroaryl represented by R⁷ are each optionally substituted with one to three R^{7a},
or alternatively two of R⁷, taken together with their intervening atoms, for a 3 to 7membered monocyclic heterocyclyl that is optionally substituted by one or two substituents independently selected from C₁₋₆ alkyl and ═O;
R^{7a}, for each occurrence, is independently selected from C₁₋₃alkyl, halo, —CN, —OR^{1c}, C₃₋₆cycloalkyl, and 4 to 6 membered monocyclic heterocycloalkyl, wherein the C₁₋₃alkyl, C₃₋₆ cycloalkyl and 4 to 6 membered monocyclic heterocycloalkyl represented by R^{7a} are each optionally substituted with one or two substituents independently selected from C₁₋₃alkyl, —OH and CN;
R^{1a} and R^{1b} are each independently H or C₁₋₄ alkyl;
R^{1c} is H or C₁₋₄ alkyl or C₁₋₄ haloalkyl;
R^{1d} is C₁₋₄ alkyl or —OR^{1e}; and
R^{1e} is C₁₋₄ alkyl; and the remaining variables are as described in the eighth, ninth, eleventh, twelfth, thirteenth, or fourteenth embodiment. In some embodiments, R⁷ is C₁₋₆alkyl, wherein one or more of the hydrogen atom is replaced by deuterium,

In a sixteenth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R⁷ is independently selected from Cl, F, —CN, —OH, —OCH₃, —O—CHF₂, —CH₃, —CD₃, —CH₂CH₃, —CH(CH₃)₂, —CHF₂, —CH₂CHF₂, —CF₃, —CH₂—OH, —CH(OH)—CH₃, —CH₂OCH₃, —CH₂CH₂OCH₂CH₃, —CH₂—CN, —N(CH₃)₂, —C(O)—NH—CH₃, —C(O)—N(CH₃)₂, —NH—C(O)—CH₃, —N(CH₃)—C(O)—O—C(CH₃)₃, —SO₂—CH₃,

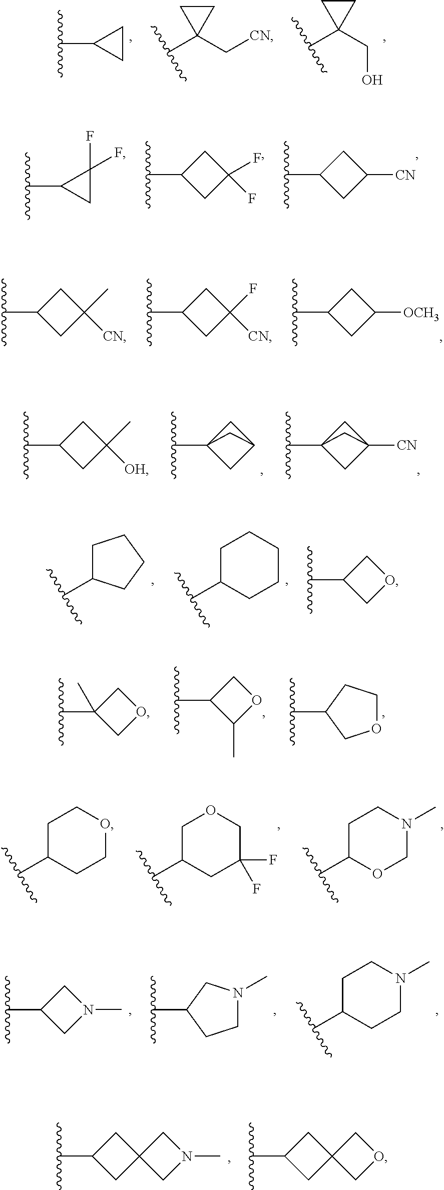

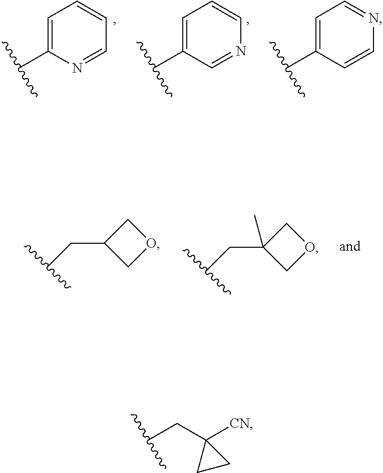

wherein

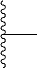

represents a bond to R^{S}; and the remaining variables are as described in e eighth, ninth, eleventh, twelfth, thirteenth, or fourteenth embodiment.

In a seventeenth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, two of R⁷, taken together with their intervening atoms, for a 3- to 7-membered monocyclic heterocyclyl that is optionally substituted by one or two substituents independently selected from C₁₋₆ alkyl and ═O; and the remaining variables are as described in the eighth, ninth, eleventh, twelfth, thirteenth, or fourteenth embodiment.

In an eighteenth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R^{S} is selected from:

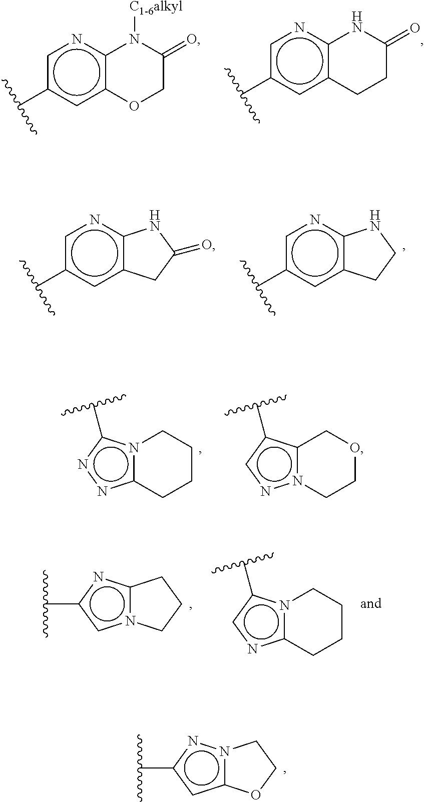

wherein

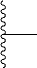

represents a bond to X₂, X₅, Y₂ or Y₅; and the remaining variables are as described in the seventeenth embodiment.

In an nineteenth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R¹, R², R³, R⁴, R⁵ and R⁶ are each independently selected from H, —CN, C₁₋₆ alkyl, C₁₋₆ haloalkyl, C₃₋₆ cycloalkyl and 4 to 6 membered heterocycloalkyl, wherein the C₁₋₆ alkyl is optionally substituted with —CN or —OR^{1f}; R^{1f} is C₁₋₄ alkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, or eighteenth embodiment.

In a twentieth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R¹, R², R³, R⁴, R⁵ and R⁶ are each independently selected from H, CN, —CH₃, —CH₂CH₃, cyclopropyl, cyclobutyl, —CHF₂, —CH₂—O—CH₃, CH₂—CN and

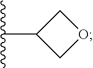 [SMILES: *C1COC1]

and the remaining variables are as described in the nineteenth embodiment.

In a twenty-first embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R¹ and R² are each independently selected from H, CN, C₁₋₆ alkyl, C₁₋₆ haloalkyl, C₃₋₆ cycloalkyl and 4 to 10 membered heterocycloalkyl, wherein the C₁₋₆ alkyl is optionally substituted with —CN or —OR^{1f}; R³ and R⁴ are each independently selected from H, C₁₋₆ alkyl and C₁₋₆ haloalkyl; R⁵ and R⁶ are each independently H or C₁₋₆ alkyl; R^{1f} is C₁₋₄ alkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, or eighteenth embodiment.

In a twenty-second embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (111-3), (III-4), or (III-5), or a pharmaceutically acceptable salt thereof, R¹ and R² are each independently selected from H, CN, —CH₃, —CH₂CH₃, —CHF₂, cyclopropyl, cyclobutyl, —CH₂—O—CH₃, —CH₂—CN and

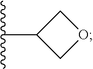 [SMILES: *C1COC1]

R³ and R⁴ are each independently selected from H, —CH₃, —CH₂CH₃ and —CHF₂; R⁵ and R⁶ are each independently selected from H, —CH₃ and —CH₂CH₃; and the remaining variables are as described in the twenty-first embodiment.

In a twenty-third embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R^{X1}, R^{X3}, R^{X4} and R^{X6} are each independently selected from H, halo, and C₁₋₄ alkyl; R^{Y1}, R^{Y3}, R^{Y4} and R^{Y6} are each independently selected from H, halo, and C₁₋₄ alkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-fourth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R^{X1}, R^{X3}, R^{X4} and R^{X6} are each independently selected from H, Cl, F, and CH₃; R^{Y1}, R^{Y3}, R^{Y4} and R^{Y6} are each independently selected from H, Cl, F, and CH₃; and the remaining variables are as described in the twenty-third embodiment.

In a twenty-fifth embodiment, for the compounds of Formula (I-1), (I-2), (I-1), (I-2), (II-1), (II-2), (II-3), (II-4), (II-5), (II-6), (II-7), (II-8), (II-9), (II-10), (II-11), (II-12), (II-13), (II-14), (II-15), (II-16), (II-17), (III-1), (III-2), (III-3), (III-4), or (III-5), or a pharmaceutically acceptable salt thereof, R^{X1}, R^{X3}, R^{X4} and R^{X6} are H; R^{Y1}, R^{Y3}, R^{Y4} and R^{Y6} are H; and the remaining variables are as described in the twenty-third embodiment.

In a twenty-sixth embodiment, the compound of the present disclosure is represented by one of the following formula:

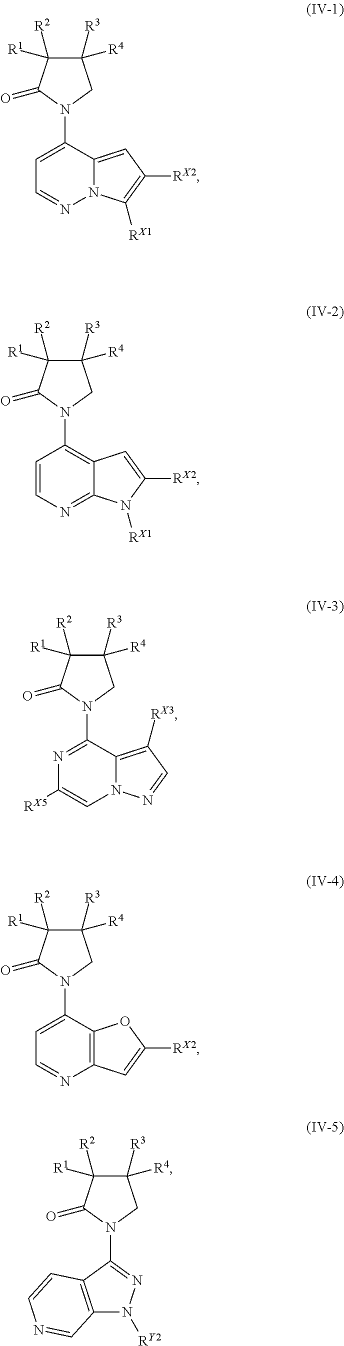

or a pharmaceutically acceptable salt thereof, wherein:
R^{X1} is H or halo;
R^{X2} is 5-membered heteroaryl optionally substituted with 1 or 2 R⁷;
R^{X3} is H or halo;
R^{X5} is 5- or 6-membered heteroaryl optionally substituted with 1 or 2 R⁷;
R^{Y2} is 5-membered heteroaryl optionally substituted with 1 or 2 R⁷;
R¹ and R² are each independently —CN or C₃₋₆ cycloalkyl; and
R³ and R⁴ are each independently H or C₁₋₃ alkyl; and the remaining variables are as described in the first embodiment.

In a twenty-seventh embodiment, for the compounds of Formula (IV-1), (IV-2), (IV-3), (IV-4), or (IV-5), or a pharmaceutically acceptable salt thereof, the 5- or 6-membered heteroaryl represented by R^{X2}, R^{X5} or R^{Y2} is pyrazole or pyridine, each of which is optionally substituted with 1 R⁷; and the remaining variables are as described in the twenty-sixth embodiment.

In a twenty-eighth embodiment, for the compounds of Formula (IV-1), (IV-2), (IV-3), (IV-4), or (IV-5), or a pharmaceutically acceptable salt thereof, the 5- or 6-membered heteroaryl represented by R^{X2}, R^{X5} or R^{Y2} is represented by one of the following formula:

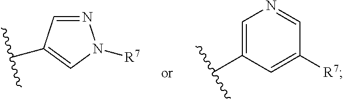

and the remaining variables are as described in the twenty-sixth embodiment.

In a twenty-ninth embodiment, for the compounds of Formula (IV-1), (IV-2), (IV-3), (IV-4), or (IV-5), or a pharmaceutically acceptable salt thereof, R⁷ is halo, C₁₋₄ alkyl, C₁₋₄ haloalkyl or 4 to 6 membered monocyclic heterocycloalkyl containing 1 or 2 heteroatoms independently selected from oxygen and nitrogen; and the remaining variables are as described in the twenty-seventh or twenty-eighth embodiment.

In a thirtieth embodiment, for the compounds of Formula (IV-1), (IV-2), (IV-3), (IV-4), or (IV-5), or a pharmaceutically acceptable salt thereof, R⁷ is F, —CH₃, —CHF₂, —CH₂CH₃ or

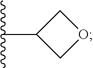 [SMILES: *C1COC1]

and the remaining variables are as described in the twenty-ninth embodiment.

In a thirty-first embodiment, for the compounds of Formula (IV-1), (IV-2), (IV-3), (IV-4), or (IV-5), or a pharmaceutically acceptable salt thereof, R¹ is CN, R² is C₃₋₆ cycloalkyl, R³ is H and R⁴ is C₁₋₃alkyl; and the remaining variables are as described in the twenty-sixth, twenty-seventh, twenty-eighth, twenty-ninth, or thirtieth embodiment.

In a thirty-second embodiment, for the compounds of Formula (IV-1), (IV-2), (IV-3), (IV-4), or (IV-5), or a pharmaceutically acceptable salt thereof, R¹ is CN, R² is cyclopropyl, R³ is H, R⁴ is —CH₃; and the remaining variables are as described in the thirty-first embodiment.

In a thirty-third embodiment, the present disclosure provides a compound described herein (e.g. a compound of any one of Examples 1 to 358), or a pharmaceutically acceptable salt thereof.

In a thirty-fourth, the present disclosure provides a compound selected from any one of the following:
(3R,4S)-3-cyclopropyl-4-methyl-1-[2-(1-methylpyrazol-4-yl)-1H-pyrrolo[2,3-b]pyridin-4-yl]-2-oxopyrrolidine-3-carbonitrile;
(3R)-3-cyclopropyl-1-[2-(1-methylpyrazol-4-yl)-1H-pyrrolo[2,3-b]pyridin-4-yl]-2-oxopyrrolidine-3-carbonitrile;
(3S)-3-cyclopropyl-1-[2-(2-methoxypyridin-4-yl)-1H-pyrrolo[2,3-b]pyridin-4-yl]-2-oxopyrrolidine-3-carbonitrile;
(3S)-3-cyclopropyl-1-[2-(1-methylpyrazol-4-yl)-1H-pyrrolo[2,3-b]pyridin-4-yl]-2-oxopyrrolidine-3-carbonitrile;
(3R)-3-cyclopropyl-1-[2-(2-methoxypyridin-4-yl)-1H-pyrrolo[2,3-b]pyridin-4-yl]-2-oxopyrrolidine-3-carbonitrile;
(3R,4S)-3-cyclopropyl-4-methyl-1-[6-(1-methylpyrazol-4-yl)-7H-pyrrolo[2,3-d]pyrimidin-4-yl]-2-oxopyrrolidine-3-carbonitrile;
rac-(3R)-1-[6-(1-methylpyrazol-4-yl)pyrrolo[1,2-b]pyridazin-4-yl]-2-oxopyrrolidine-3-carbonitrile;
(3S)-3-cyclopropyl-1-[6-(2-methoxypyridin-4-yl)pyrrolo[1,2-b]pyridazin-4-yl]-2-oxopyrrolidine-3-carbonitrile;
(3S)-3-cyclopropyl-1-[6-(1-methylpyrazol-4-yl)pyrrolo[1,2-b]pyridazin-4-yl]-2-oxopyrrolidine-3-carbonitrile;
(3S)-3-cyclopropyl-1-[6-[1-(difluoromethyl)pyrazol-4-yl]pyrrolo[1,2-b]pyridazin-4-yl]-2-oxopyrrolidine-3-carbonitrile;
(3S)-3-cyclopropyl-1-[6-(3-methyl-1,2-oxazol-5-yl)pyrrolo[1,2-b]pyridazin-4-yl]-2-oxopyrrolidine-3-carbonitrile;
(3S)-3-cyclopropyl-1-[6-(3-fluoro-1-methylpyrazol-4-yl)pyrrolo[1,2-b]pyridazin-4-yl]-2-oxopyrrolidine-3-carbonitrile;
(3R)-3-cyclopropyl-1-[6-(2-methoxypyridin-4-yl)pyrrolo[1,2-b]pyridazin-4-yl]-2-oxopyrrolidine-3-carbonitrile;
(3R)-3-cyclopropyl-1-[6-(1-methylpyrazol-4-yl)pyrrolo[1,2-b]pyridazin-4-yl]-2-oxopyrrolidine-3-carbonitrile;
(3R)-3-cyclopropyl-3-(difluoromethyl)-1-[6-(1-methylpyrazol-4-yl)pyrrolo[1,2-b]pyridazin-4-yl]pyrrolidin-2-one;
(3R,5R)-3-cyclopropyl-5-methyl-1-[6-(1-methylpyrazol-4-yl)pyrrolo[1,2-b]pyridazin-4-yl]-2-oxopyrrolidine-3-carbonitrile;
(3S,5R)-3-cyclopropyl-5-methyl-1-[6-(1-methylpyrazol-4-yl)pyrrolo[1,2-b]pyridazin-4-yl]-2-oxopyrrolidine-3-carbonitrile;
(3S,5R)-3-cyclopropyl-5-ethyl-1-[6-(1-methylpyrazol-4-yl)pyrrolo[1,2-b]pyridazin-4-yl]-2-oxopyrrolidine-3-carbonitrile;
(3S,5S)-3-cyclopropyl-5-ethyl-1-[6-(2-methyl-2H-imidazol-4-yl)pyrrolo[1,2-b]pyridazin-4-yl]-2-oxopyrrolidine-3-carbonitrile;
(3S,4S)-4-methyl-1-[6-(1-methylpyrazol-4-yl)pyrrolo[1,2-b]pyridazin-4-yl]-3-(oxetan-3-yl)-2-oxopyrrolidine-3-carbonitrile;
(3S,4S)-3-cyclopropyl-4-methyl-1-[6-(1-methylpyrazol-4-yl)pyrrolo[1,2-b]pyridazin-4-yl]-2-oxopyrrolidine-3-carbonitrile;
(3R,4R)-3-cyclopropyl-4-methyl-1-[6-(1-methylpyrazol-4-yl)pyrrolo[1,2-b]pyridazin-4-yl]-2-oxopyrrolidine-3-carbonitrile;
(3R,4S)-3-cyclopropyl-4-ethyl-1-[6-(1-methylpyrazol-4-yl)pyrrolo[1,2-b]pyridazin-4-yl]-2-oxopyrrolidine-3-carbonitrile;
(3R,4R)-3-cyclopropyl-4-ethyl-1-[6-(1-methylpyrazol-4-yl)pyrrolo[1,2-b]pyridazin-4-yl]-2-oxopyrrolidine-3-carbonitrile;
(3S,4R)-3-cyclopropyl-4-ethyl-1-[6-(1-methylpyrazol-4-yl)pyrrolo[1,2-b]pyridazin-4-yl]-2-oxopyrrolidine-3-carbonitrile;
(3S,4S)-3-cyclopropyl-4-ethyl-1-[6-(1-methylpyrazol-4-yl)pyrrolo[1,2-b]pyridazin-4-yl]-2-oxopyrrolidine-3-carbonitrile;
2-[(3R)-3-cyclopropyl-1-[6-(1-methylpyrazol-4-yl)pyrrolo[1,2-b]pyridazin-4-yl]-2-oxopyrrolidin-3-yl]acetonitrile;
(3R,4S)-3-cyclopropyl-1-[7-fluoro-6-(1-methylpyrazol-4-yl)pyrrolo[1,2-b]pyridazin-4-yl]-4-methyl-2-oxopyrrolidine-3-carbonitrile;
(3S,4R)-3-cyclopropyl-4-methyl-1-[6-(1-methylpyrazol-4-yl)pyrrolo[1,2-b]pyridazin-4-yl]-2-oxopyrrolidine-3-carbonitrile;
(3R,5S)-3-cyclopropyl-5-methyl-1-[6-(1-methylpyrazol-4-yl)pyrrolo[1,2-b]pyridazin-4-yl]-2-oxopyrrolidine-3-carbonitrile;
(3S,5S)-3-cyclopropyl-5-methyl-1-[6-(1-methylpyrazol-4-yl)pyrrolo[1,2-b]pyridazin-4-yl]-2-oxopyrrolidine-3-carbonitrile;
(3R,5S)-3-cyclopropyl-5-ethyl-1-[6-(1-methylpyrazol-4-yl)pyrrolo[1,2-b]pyridazin-4-yl]-2-oxopyrrolidine-3-carbonitrile;
(3R,5R)-3-cyclopropyl-5-ethyl-1-[6-(1-methylpyrazol-4-yl)pyrrolo[1,2-b]pyridazin-4-yl]-2-oxopyrrolidine-3-carbonitrile;
(3R,4S)-3-cyclopropyl-4-methyl-1-[6-(1-methylpyrazol-4-yl)pyrrolo[1,2-b]pyridazin-4-yl]-2-oxopyrrolidine-3-carbonitrile;
(3R,4S)-3-cyclopropyl-1-[6-[1-(difluoromethyl)pyrazol-4-yl]pyrrolo[1,2-b]pyridazin-4-yl]-4-methyl-2-oxopyrrolidine-3-carbonitrile;
rel-(3R)-3-cyclopropyl-1-[6-(1-methylpyrazol-4-yl)pyrrolo[1,2-b]pyridazin-4-yl]pyrrolidin-2-one;
rel-(3R)-3-cyclopropyl-1-[6-(1-methylpyrazol-4-yl)pyrrolo[1,2-b]pyridazin-4-yl]pyrrolidin-2-one;
(3S)-3-methyl-1-[6-(1-methylpyrazol-4-yl)pyrrolo[1,2-b]pyridazin-4-yl]-2-oxopyrrolidine-3-carbonitrile;
(3R)-3-methyl-1-[6-(1-methylpyrazol-4-yl)pyrrolo[1,2-b]pyridazin-4-yl]-2-oxopyrrolidine-3-carbonitrile;
rel-(3R)-3-(methoxymethyl)-1-[6-(1-methylpyrazol-4-yl)pyrrolo[1,2-b]pyridazin-4-yl]-2-oxopyrrolidine-3-carbonitrile;
rel-(3R)-3-(methoxymethyl)-1-[6-(1-methylpyrazol-4-yl)pyrrolo[1,2-b]pyridazin-4-yl]-2-oxopyrrolidine-3-carbonitrile;
rel-(3R)-3-ethyl-1-[6-(1-methylpyrazol-4-yl)pyrrolo[1,2-b]pyridazin-4-yl]-2-oxopyrrolidine-3-carbonitrile;
rel-(3R)-3-ethyl-1-[6-(1-methylpyrazol-4-yl)pyrrolo[1,2-b]pyridazin-4-yl]-2-oxopyrrolidine-3-carbonitrile;
rel-(3R)-1-[6-(1-methylpyrazol-4-yl)pyrrolo[1,2-b]pyridazin-4-yl]-3-(oxetan-3-yl)-2-oxopyrrolidine-3-carbonitrile;
rel-(3R)-1-[6-(1-methylpyrazol-4-yl)pyrrolo[1,2-b]pyridazin-4-yl]-3-(oxetan-3-yl)-2-oxopyrrolidine-3-carbonitrile;
rel-(3R)-3-cyclobutyl-1-[6-(1-methylpyrazol-4-yl)pyrrolo[1,2-b]pyridazin-4-yl]-2-oxopyrrolidine-3-carbonitrile;
rel-(3R)-3-cyclobutyl-1-[6-(1-methylpyrazol-4-yl)pyrrolo[1,2-b]pyridazin-4-yl]-2-oxopyrrolidine-3-carbonitrile;
(3R,4S)-3-cyclopropyl-1-[6-(6-methoxypyridin-3-yl)pyrrolo[1,2-b]pyridazin-4-yl]-4-methyl-2-oxopyrrolidine-3-carbonitrile;
(3R,4S)-3-cyclopropyl-4-methyl-1-[6-(2-methylpyrazol-3-yl)pyrrolo[1,2-b]pyridazin-4-yl]-2-oxopyrrolidine-3-carbonitrile;
(3R,4S)-3-cyclopropyl-1-[6-[2-(difluoromethoxy)pyridin-3-yl]pyrrolo[1,2-b]pyridazin-4-yl]-4-methyl-2-oxopyrrolidine-3-carbonitrile;
(3R,4S)-3-cyclopropyl-1-(6-imidazo[1,5-a]pyridin-6-ylpyrrolo[1,2-b]pyridazin-4-yl)-4-methyl-2-oxopyrrolidine-3-carbonitrile;
(3R,4S)-3-cyclopropyl-4-methyl-1-[6-(6-methylsulfonylpyridin-3-yl)pyrrolo[1,2-b]pyridazin-4-yl]-2-oxopyrrolidine-3-carbonitrile;
(3R,4S)-3-cyclopropyl-1-[6-(5-hydroxypyridin-3-yl)pyrrolo[1,2-b]pyridazin-4-yl]-4-methyl-2-oxopyrrolidine-3-carbonitrile;
(3R,4S)-3-cyclopropyl-4-methyl-1-[6-(1-methylindazol-4-yl)pyrrolo[1,2-b]pyridazin-4-yl]-2-oxopyrrolidine-3-carbonitrile;
(3R,4S)-3-cyclopropyl-4-methyl-1-[6-(1-methylindazol-5-yl)pyrrolo[1,2-b]pyridazin-4-yl]-2-oxopyrrolidine-3-carbonitrile;
(3R,4S)-3-cyclopropyl-1-[6-(2-fluoropyridin-3-yl)pyrrolo[1,2-b]pyridazin-4-yl]-4-methyl-2-oxopyrrolidine-3-carbonitrile;
(3R,4S)-3-cyclopropyl-4-methyl-2-oxo-1-[6-([1,2,4]triazolo[1,5-a]pyridin-6-yl)pyrrolo[1,2-b]pyridazin-4-yl]pyrrolidine-3-carbonitrile;
5-[4-[(3R,4S)-3-cyano-3-cyclopropyl-4-methyl-2-oxopyrrolidin-1-yl]pyrrolo[1,2-b]pyridazin-6-yl]-N-methylpyridine-2-carboxamide;
(3R,4S)-3-cyclopropyl-4-methyl-2-oxo-1-[6-(1H-pyrazol-4-yl)pyrrolo[1,2-b]pyridazin-4-yl]pyrrolidine-3-carbonitrile;2,2,2-trifluoroacetic acid;
(3R,4S)-3-cyclopropyl-4-methyl-2-oxo-1-(6-pyridin-3-ylpyrrolo[1,2-b]pyridazin-4-yl)pyrrolidine-3-carbonitrile;
N-[4-[4-[(3R,4S)-3-cyano-3-cyclopropyl-4-methyl-2-oxopyrrolidin-1-yl]pyrrolo[1,2-b]pyridazin-6-yl]pyridin-2-yl]acetamide;
(3R,4S)-3-cyclopropyl-1-[6-[6-(dimethylamino)pyridin-3-yl]pyrrolo[1,2-b]pyridazin-4-yl]-4-methyl-2-oxopyrrolidine-3-carbonitrile;
(3R,4S)-3-cyclopropyl-4-methyl-1-[6-(1-methylpyrazol-3-yl)pyrrolo[1,2-b]pyridazin-4-yl]-2-oxopyrrolidine-3-carbonitrile;
(3R,4S)-3-cyclopropyl-4-methyl-2-oxo-1-[6-(1H-pyrrolo[2,3-b]pyridin-5-yl)pyrrolo[1,2-b]pyridazin-4-yl]pyrrolidine-3-carbonitrile;
(3R,4S)-3-cyclopropyl-1-[6-(2-methoxypyridin-4-yl)pyrrolo[1,2-b]pyridazin-4-yl]-4-methyl-2-oxopyrrolidine-3-carbonitrile;
(3R,4S)-3-cyclopropyl-4-methyl-2-oxo-1-[6-(1H-pyrazol-3-yl)pyrrolo[1,2-b]pyridazin-4-yl]pyrrolidine-3-carbonitrile;2,2,2-trifluoroacetic acid;
(3R,4S)-3-cyclopropyl-4-methyl-1-[6-(6-methylpyridin-3-yl)pyrrolo[1,2-b]pyridazin-4-yl]-2-oxopyrrolidine-3-carbonitrile;
(3R,4S)-3-cyclopropyl-4-methyl-1-[6-(1-methylindazol-6-yl)pyrrolo[1,2-b]pyridazin-4-yl]-2-oxopyrrolidine-3-carbonitrile;
(3R,4S)-3-cyclopropyl-4-methyl-2-oxo-1-[6-[2-(trifluoromethyl)pyridin-4-yl]pyrrolo[1,2-b]pyridazin-4-yl]pyrrolidine-3-carbonitrile;
(3R,4S)-3-cyclopropyl-4-methyl-2-oxo-1-[6-[5-(trifluoromethyl)pyridin-3-yl]pyrrolo[1,2-b]pyridazin-4-yl]pyrrolidine-3-carbonitrile;
(3R,4S)-3-cyclopropyl-4-methyl-1-[6-(5-methylpyridin-3-yl)pyrrolo[1,2-b]pyridazin-4-yl]-2-oxopyrrolidine-3-carbonitrile;
(3R,4S)-3-cyclopropyl-4-methyl-2-oxo-1-[6-(1H-pyrrolo[2,3-b]pyridin-3-yl)pyrrolo[1,2-b]pyridazin-4-yl]pyrrolidine-3-carbonitrile;
(3R,4S)-3-cyclopropyl-1-[6-(2-fluoropyridin-4-yl)pyrrolo[1,2-b]pyridazin-4-yl]-4-methyl-2-oxopyrrolidine-3-carbonitrile;
(3R,4S)-3-cyclopropyl-4-methyl-2-oxo-1-[6-[6-(trifluoromethyl)pyridin-3-yl]pyrrolo[1,2-b]pyridazin-4-yl]pyrrolidine-3-carbonitrile;
(3R,4S)-3-cyclopropyl-4-methyl-2-oxo-1-[6-(1H-pyrazolo[3,4-b]pyridin-5-yl)pyrrolo[1,2-b]pyridazin-4-yl]pyrrolidine-3-carbonitrile;
(3R,4S)-3-cyclopropyl-1-[6-(2-methoxypyrimidin-5-yl)pyrrolo[1,2-b]pyridazin-4-yl]-4-methyl-2-oxopyrrolidine-3-carbonitrile;
(3R,4S)-3-cyclopropyl-4-methyl-1-[6-(2-methylpyridin-4-yl)pyrrolo[1,2-b]pyridazin-4-yl]-2-oxopyrrolidine-3-carbonitrile;
(3R,4S)-3-cyclopropyl-4-methyl-1-[6-(1-methylindazol-7-yl)pyrrolo[1,2-b]pyridazin-4-yl]-2-oxopyrrolidine-3-carbonitrile;
(3R,4S)-3-cyclopropyl-4-methyl-1-[6-(1-methyl-2-oxopyridin-4-yl)pyrrolo[1,2-b]pyridazin-4-yl]-2-oxopyrrolidine-3-carbonitrile;
(3R,4S)-3-cyclopropyl-4-methyl-1-[6-(2-methylpyrimidin-5-yl)pyrrolo[1,2-b]pyridazin-4-yl]-2-oxopyrrolidine-3-carbonitrile;
(3R,4S)-3-cyclopropyl-1-[6-(6-fluoropyridin-3-yl)pyrrolo[1,2-b]pyridazin-4-yl]-4-methyl-2-oxopyrrolidine-3-carbonitrile;
(3R,4S)-3-cyclopropyl-1-[6-(5-methoxypyridin-3-yl)pyrrolo[1,2-b]pyridazin-4-yl]-4-methyl-2-oxopyrrolidine-3-carbonitrile;
(3R,4S)-3-cyclopropyl-4-methyl-1-[6-(1-methyl-6-oxopyridin-3-yl)pyrrolo[1,2-b]pyridazin-4-yl]-2-oxopyrrolidine-3-carbonitrile;
(3R,4S)-3-cyclopropyl-1-[6-(5-fluoropyridin-3-yl)pyrrolo[1,2-b]pyridazin-4-yl]-4-methyl-2-oxopyrrolidine-3-carbonitrile;
5-[4-[(3R,4S)-3-cyano-3-cyclopropyl-4-methyl-2-oxopyrrolidin-1-yl]pyrrolo[1,2-b]pyridazin-6-yl]-2-methoxypyridine-3-carbonitrile;
5-[4-[(3R,4S)-3-cyano-3-cyclopropyl-4-methyl-2-oxopyrrolidin-1-yl]pyrrolo[1,2-b]pyridazin-6-yl]pyridine-2-carbonitrile;2,2,2-trifluoroacetic acid;
(3R,4S)-1-[6-(5-chloropyridin-3-yl)pyrrolo[1,2-b]pyridazin-4-yl]-3-cyclopropyl-4-methyl-2-oxopyrrolidine-3-carbonitrile;
(3R,4S)-3-cyclopropyl-1-(6-imidazo[1,2-a]pyridin-6-ylpyrrolo[1,2-b]pyridazin-4-yl)-4-methyl-2-oxopyrrolidine-3-carbonitrile;
(3R,4S)-3-cyclopropyl-4-methyl-1-[6-(3-methylimidazol-4-yl)pyrrolo[1,2-b]pyridazin-4-yl]-2-oxopyrrolidine-3-carbonitrile;
N-[5-[4-[(3R,4S)-3-cyano-3-cyclopropyl-4-methyl-2-oxopyrrolidin-1-yl]pyrrolo[1,2-b]pyridazin-6-yl]pyridin-3-yl]acetamide;
(3R,4S)-3-cyclopropyl-1-[6-[2-(dimethylamino)pyrimidin-5-yl]pyrrolo[1,2-b]pyridazin-4-yl]-4-methyl-2-oxopyrrolidine-3-carbonitrile;
(3R,4S)-3-cyclopropyl-4-methyl-2-oxo-1-(6-pyrimidin-5-ylpyrrolo[1,2-b]pyridazin-4-yl)pyrrolidine-3-carbonitrile;
4-[4-[(3R,4S)-3-cyano-3-cyclopropyl-4-methyl-2-oxopyrrolidin-1-yl]pyrrolo[1,2-b]pyridazin-6-yl]pyridine-2-carbonitrile;
(3R,4S)-3-cyclopropyl-1-[6-(5-fluoro-2-methoxypyridin-4-yl)pyrrolo[1,2-b]pyridazin-4-yl]-4-methyl-2-oxopyrrolidine-3-carbonitrile;
tert-butyl N-[5-[4-[(3R,4S)-3-cyano-3-cyclopropyl-4-methyl-2-oxopyrrolidin-1-yl]pyrrolo[1,2-b]pyridazin-6-yl]pyridin-2-yl]-N-methylcarbamate;
(3R,4S)-3-cyclopropyl-1-[6-(2-methoxypyridin-3-yl)pyrrolo[1,2-b]pyridazin-4-yl]-4-methyl-2-oxopyrrolidine-3-carbonitrile;
(3R,4S)-3-cyclopropyl-4-methyl-1-[6-[1-(oxetan-3-yl)pyrazol-4-yl]pyrrolo[1,2-b]pyridazin-4-yl]-2-oxopyrrolidine-3-carbonitrile;
(3R,4S)-3-cyclopropyl-1-[6-(3,6-dimethoxypyridazin-4-yl)pyrrolo[1,2-b]pyridazin-4-yl]-4-methyl-2-oxopyrrolidine-3-carbonitrile;
(3R,4S)-3-cyclopropyl-4-methyl-1-[6-(2-methyltriazol-4-yl)pyrrolo[1,2-b]pyridazin-4-yl]-2-oxopyrrolidine-3-carbonitrile;2,2,2-trifluoroacetic acid;
(3R,4S)-3-cyclopropyl-4-methyl-2-oxo-1-[6-(5,6,7,8-tetrahydroimidazo[1,2-a]pyridin-3-yl)pyrrolo[1,2-b]pyridazin-4-yl]pyrrolidine-3-carbonitrile;2,2,2-trifluoroacetic acid;
(3R,4S)-3-cyclopropyl-1-[6-[2-(difluoromethyl)-1,3-thiazol-5-yl]pyrrolo[1,2-b]pyridazin-4-yl]-4-methyl-2-oxopyrrolidine-3-carbonitrile;2,2,2-trifluoroacetic acid;
(3R,4S)-3-cyclopropyl-4-methyl-2-oxo-1-[6-([1,3]thiazolo[4,5-c]pyridin-2-yl)pyrrolo[1,2-b]pyridazin-4-yl]pyrrolidine-3-carbonitrile;2,2,2-trifluoroacetic acid;
3-[4-[(3R,4S)-3-cyano-3-cyclopropyl-4-methyl-2-oxopyrrolidin-1-yl]pyrrolo[1,2-b]pyridazin-6-yl]imidazo[1,2-a]pyridine-7-carbonitrile;2,2,2-trifluoroacetic acid;
(3R,4S)-3-cyclopropyl-4-methyl-2-oxo-1-(6-pyrazolo[1,5-a]pyridin-2-ylpyrrolo[1,2-b]pyridazin-4-yl)pyrrolidine-3-carbonitrile;2,2,2-trifluoroacetic acid;
(3R,4S)-3-cyclopropyl-4-methyl-2-oxo-1-[6-(5,6,7,8-tetrahydro-[1,2,4]triazolo[4,3-a]pyridin-3-yl)pyrrolo[1,2-b]pyridazin-4-yl]pyrrolidine-3-carbonitrile;2,2,2-trifluoroacetic acid;
(3R,4S)-3-cyclopropyl-1-[6-(6,7-dihydro-5H-pyrrolo[1,2-a]imidazol-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-4-methyl-1-[6-[2-methyl-5-(trifluoromethyl)pyrazol-3-yl]pyrrolo[1,2-b]pyridazin-4-yl]-2-oxopyrrolidine-3-carbonitrile;2,2,2-trifluoroacetic acid;
(3R,4S)-3-cyclopropyl-4-methyl-2-oxo-1-[6-([1,2,4]triazolo[1,5-a]pyridin-2-yl)pyrrolo[1,2-b]pyridazin-4-yl]pyrrolidine-3-carbonitrile;2,2,2-trifluoroacetic acid;
(3R,4S)-3-cyclopropyl-4-methyl-1-[6-(6-methyl-[1,2,4]triazolo[1,5-a]pyridin-2-yl)pyrrolo[1,2-b]pyridazin-4-yl]-2-oxopyrrolidine-3-carbonitrile;2,2,2-trifluoroacetic acid;
(3R,4S)-3-cyclopropyl-1-[6-(2,3-dihydropyrazolo[5,1-b][1,3]oxazol-6-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-4-methyl-1-[6-(1-methylimidazol-4-yl)pyrrolo[1,2-b]pyridazin-4-yl]-2-oxopyrrolidine-3-carbonitrile;2,2,2-trifluoroacetic acid;
(3R,4S)-3-cyclopropyl-4-methyl-1-[6-(1-methyltriazol-4-yl)pyrrolo[1,2-b]pyridazin-4-yl]-2-oxopyrrolidine-3-carbonitrile;2,2,2-trifluoroacetic acid;
(3R,4S)-3-cyclopropyl-4-methyl-1-[6-(4-methyl-1,3-thiazol-2-yl)pyrrolo[1,2-b]pyridazin-4-yl]-2-oxopyrrolidine-3-carbonitrile;2,2,2-trifluoroacetic acid;
2-[4-[(3R,4S)-3-cyano-3-cyclopropyl-4-methyl-2-oxopyrrolidin-1-yl]pyrrolo[1,2-b]pyridazin-6-yl]-1,3-thiazole-4-carbonitrile;2,2,2-trifluoroacetic acid;
(3R,4S)-3-cyclopropyl-4-methyl-1-[6-(1-methyl-1,2,4-triazol-3-yl)pyrrolo[1,2-b]pyridazin-4-yl]-2-oxopyrrolidine-3-carbonitrile;2,2,2-trifluoroacetic acid;
(3R,4S)-3-cyclopropyl-1-(6-imidazo[1,2-a]pyrazin-3-ylpyrrolo[1,2-b]pyridazin-4-yl)-4-methyl-2-oxopyrrolidine-3-carbonitrile;2,2,2-trifluoroacetic acid;
2-[4-[(3R,4S)-3-cyano-3-cyclopropyl-4-methyl-2-oxopyrrolidin-1-yl]pyrrolo[1,2-b]pyridazin-6-yl]-1,3-thiazole-5-carbonitrile;2,2,2-trifluoroacetic acid;
(3R,4S)-3-cyclopropyl-4-methyl-1-[6-(5-methyl-1,3-thiazol-2-yl)pyrrolo[1,2-b]pyridazin-4-yl]-2-oxopyrrolidine-3-carbonitrile;2,2,2-trifluoroacetic acid;
6-[4-[(3R,4S)-3-cyano-3-cyclopropyl-4-methyl-2-oxopyrrolidin-1-yl]pyrrolo[1,2-b]pyridazin-6-yl]-2-methylpyridine-3-carbonitrile;2,2,2-trifluoroacetic acid;
(3R,4S)-3-cyclopropyl-4-methyl-2-oxo-1-[6-[4-(trifluoromethyl)pyrimidin-2-yl]pyrrolo[1,2-b]pyridazin-4-yl]pyrrolidine-3-carbonitrile;2,2,2-trifluoroacetic acid;
(3R,4S)-3-cyclopropyl-4-methyl-2-oxo-1-[6-[6-(trifluoromethyl)pyrimidin-4-yl]pyrrolo[1,2-b]pyridazin-4-yl]pyrrolidine-3-carbonitrile;2,2,2-trifluoroacetic acid;
(3R,4S)-3-cyclopropyl-1-[6-(4-methoxypyrimidin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-(6-imidazo[1,2-a]pyrazin-8-ylpyrrolo[1,2-b]pyridazin-4-yl)-4-methyl-2-oxopyrrolidine-3-carbonitrile;2,2,2-trifluoroacetic acid;
6-[4-[(3R,4S)-3-cyano-3-cyclopropyl-4-methyl-2-oxopyrrolidin-1-yl]pyrrolo[1,2-b]pyridazin-6-yl]-N,N-dimethylpyridine-3-carboxamide;2,2,2-trifluoroacetic acid;
(3R,4S)-1-[6-[6-(cyanomethyl)pyridin-2-yl]pyrrolo[1,2-b]pyridazin-4-yl]-3-cyclopropyl-4-methyl-2-oxopyrrolidine-3-carbonitrile;2,2,2-trifluoroacetic acid;
(3R,4S)-3-cyclopropyl-1-[6-(6-methoxypyrazin-2-yl)pyrrolo[1,2-b]pyridazin-4-yl]-4-methyl-2-oxopyrrolidine-3-carbonitrile;2,2,2-trifluoroacetic acid;
(3R,4S)-1-[6-[5-(cyanomethyl)pyridin-2-yl]pyrrolo[1,2-b]pyridazin-4-yl]-3-cyclopropyl-4-methyl-2-oxopyrrolidine-3-carbonitrile;2,2,2-trifluoroacetic acid;
(3R,4S)-3-cyclopropyl-1-(6-imidazo[1,5-a]pyridin-5-ylpyrrolo[1,2-b]pyridazin-4-yl)-4-methyl-2-oxopyrrolidine-3-carbonitrile;2,2,2-trifluoroacetic acid;
2-[4-[(3R,4S)-3-cyano-3-cyclopropyl-4-methyl-2-oxopyrrolidin-1-yl]pyrrolo[1,2-b]pyridazin-6-yl]-4-hydroxypyridine-3-carbonitrile;2,2,2-trifluoroacetic acid;
(3R,4S)-3-cyclopropyl-1-[6-[5-(1-hydroxyethyl)pyridin-2-yl]pyrrolo[1,2-b]pyridazin-4-yl]-4-methyl-2-oxopyrrolidine-3-carbonitrile;2,2,2-trifluoroacetic acid;
N-[6-[4-[(3R,4S)-3-cyano-3-cyclopropyl-4-methyl-2-oxopyrrolidin-1-yl]pyrrolo[1,2-b]pyridazin-6-yl]pyridin-2-yl]acetamide;2,2,2-trifluoroacetic acid;
(3R,4S)-3-cyclopropyl-1-[6-(5-methoxypyrazin-2-yl)pyrrolo[1,2-b]pyridazin-4-yl]-4-methyl-2-oxopyrrolidine-3-carbonitrile;2,2,2-trifluoroacetic acid;
6-[4-[(3R,4S)-3-cyano-3-cyclopropyl-4-methyl-2-oxopyrrolidin-1-yl]pyrrolo[1,2-b]pyridazin-6-yl]pyrazine-2-carbonitrile;2,2,2-trifluoroacetic acid;
(3R,4S)-3-cyclopropyl-1-[6-[5-(dimethylamino)pyridin-2-yl]pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-[6-(difluoromethyl)pyridin-2-yl]pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-(5-methoxypyrimidin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-4-methyl-1-[6-(6-methylpyridazin-3-yl)pyrrolo[1,2-b]pyridazin-4-yl]-2-oxopyrrolidine-3-carbonitrile;2,2,2-trifluoroacetic acid;
(3R,4S)-3-cyclopropyl-1-[6-(3-fluoropyridin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-4-methyl-2-oxo-1-(6-pyrimidin-2-ylpyrrolo[1,2-b]pyridazin-4-yl)pyrrolidine-3-carbonitrile;2,2,2-trifluoroacetic acid;
(3R,4S)-3-cyclopropyl-1-[6-(3-hydroxy-6-methylpyridin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-(6-imidazo[1,2-a]pyridin-5-ylpyrrolo[1,2-b]pyridazin-4-yl)-4-methyl-2-oxopyrrolidine-3-carbonitrile;2,2,2-trifluoroacetic acid;
(3R,4S)-3-cyclopropyl-1-[6-(5-fluoro-4-methylpyridin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-(6-methoxypyridin-2-yl)pyrrolo[1,2-b]pyridazin-4-yl]-4-methyl-2-oxopyrrolidine-3-carbonitrile;2,2,2-trifluoroacetic acid;
6-[4-[(3R,4S)-3-cyano-3-cyclopropyl-4-methyl-2-oxopyrrolidin-1-yl]pyrrolo[1,2-b]pyridazin-6-yl]-4-methylpyridine-3-carbonitrile;2,2,2-trifluoroacetic acid;
(3R,4S)-3-cyclopropyl-1-[6-(5-methoxypyridin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-(3-methoxypyridin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-(5-fluoro-6-methylpyridin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-[4-(hydroxymethyl)pyridin-2-yl]pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-(3-hydroxypyridin-2-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-[5-(hydroxymethyl)pyridin-2-yl]pyrrolo[1,2-b]pyridazin-4-yl]-4-methyl-2-oxopyrrolidine-3-carbonitrile;2,2,2-trifluoroacetic acid;
(3R,4S)-3-cyclopropyl-4-methyl-2-oxo-1-(6-pyrazin-2-ylpyrrolo[1,2-b]pyridazin-4-yl)pyrrolidine-3-carbonitrile;2,2,2-trifluoroacetic acid;
rac-(3R,4R)-3-cyclopropyl-4-(difluoromethyl)-1-[6-(1-methylpyrazol-4-yl)pyrrolo[1,2-b]pyridazin-4-yl]-2-oxopyrrolidine-3-carbonitrile;
rac-(3R,4S)-3-cyclopropyl-4-(difluoromethyl)-1-[6-(1-methylpyrazol-4-yl)pyrrolo[1,2-b]pyridazin-4-yl]-2-oxopyrrolidine-3-carbonitrile;
(3R,4S)-3-cyclopropyl-1-[6-[1-[(1R^{S})-2,2-difluorocyclopropyl]pyrazol-4-yl]pyrrolo[1,2-b]pyridazin-4-yl]-4-methyl-2-oxopyrrolidine-3-carbonitrile;
(3R,4S)-3-cyclopropyl-1-[6-[1-[(1R*)-2,2-difluorocyclopropyl]pyrazol-4-yl]pyrrolo[1,2-b]pyridazin-4-yl]-4-methyl-2-oxopyrrolidine-3-carbonitrile;
(3R,4S)-3-cyclopropyl-1-[6-[1-[(1R*)-2,2-difluorocyclopropyl]pyrazol-4-yl]pyrrolo[1,2-b]pyridazin-4-yl]-4-methyl-2-oxopyrrolidine-3-carbonitrile;
(3R,4S)-3-cyclopropyl-1-[6-[1-(3,3-difluorocyclobutyl)pyrazol-4-yl]pyrrolo[1,2-b]pyridazin-4-yl]-4-methyl-2-oxopyrrolidine-3-carbonitrile;
(3R,4S)-3-cyclopropyl-4-methyl-2-oxo-1-[6-(1-pyridin-2-ylpyrazol-4-yl)pyrrolo[1,2-b]pyridazin-4-yl]pyrrolidine-3-carbonitrile;
(3R,4S)-3-cyclopropyl-4-methyl-2-oxo-1-[6-(1-pyridin-3-ylpyrazol-4-yl)pyrrolo[1,2-b]pyridazin-4-yl]pyrrolidine-3-carbonitrile;
(3R,4S)-3-cyclopropyl-4-methyl-2-oxo-1-[6-(1-pyridin-4-ylpyrazol-4-yl)pyrrolo[1,2-b]pyridazin-4-yl]pyrrolidine-3-carbonitrile;
(3R,4S)-3-cyclopropyl-1-[6-(1-cyclopropylpyrazol-4-yl)pyrrolo[1,2-b]pyridazin-4-yl]-4-methyl-2-oxopyrrolidine-3-carbonitrile;
(3R,4S)-3-cyclopropyl-4-methyl-2-oxo-1-[6-[1-[(3RS)-oxolan-3-yl]pyrazol-4-yl]pyrrolo[1,2-b]pyridazin-4-yl]pyrrolidine-3-carbonitrile;
(3R,4S)-3-cyclopropyl-4-methyl-1-[6-(1-methylsulfonylpyrazol-4-yl)pyrrolo[1,2-b]pyridazin-4-yl]-2-oxopyrrolidine-3-carbonitrile;
(3R,4S)-3-cyclopropyl-4-methyl-2-oxo-1-[6-[1-(trideuteriomethyl)pyrazol-4-yl]pyrrolo[1,2-b]pyridazin-4-yl]pyrrolidine-3-carbonitrile;
(3R,4S)-3-cyclopropyl-1-[6-(1,3-dimethylpyrazol-4-yl)pyrrolo[1,2-b]pyridazin-4-yl]-4-methyl-2-oxopyrrolidine-3-carbonitrile;
(3R,4S)-3-cyclopropyl-1-[6-(1,5-dimethylpyrazol-4-yl)pyrrolo[1,2-b]pyridazin-4-yl]-4-methyl-2-oxopyrrolidine-3-carbonitrile;
(3R,4S)-3-cyclopropyl-4-methyl-2-oxo-1-[6-(1-propan-2-ylpyrazol-4-yl)pyrrolo[1,2-b]pyridazin-4-yl]pyrrolidine-3-carbonitrile;
(3R,4S)-1-[6-[1-(cyanomethyl)pyrazol-4-yl]pyrrolo[1,2-b]pyridazin-4-yl]-3-cyclopropyl-4-methyl-2-oxopyrrolidine-3-carbonitrile;
(3R,4S)-1-[6-(1-cyclohexylpyrazol-4-yl)pyrrolo[1,2-b]pyridazin-4-yl]-3-cyclopropyl-4-methyl-2-oxopyrrolidine-3-carbonitrile;
(3R,4S)-1-[6-(1-cyclopentylpyrazol-4-yl)pyrrolo[1,2-b]pyridazin-4-yl]-3-cyclopropyl-4-methyl-2-oxopyrrolidine-3-carbonitrile;
(3R,4S)-3-cyclopropyl-1-[6-(5,6-dihydro-4H-pyrrolo[1,2-b]pyrazol-3-yl)pyrrolo[1,2-b]pyridazin-4-yl]-4-methyl-2-oxopyrrolidine-3-carbonitrile;
(3R,4S)-3-cyclopropyl-1-[6-[1-(2-ethoxyethyl)pyrazol-4-yl]pyrrolo[1,2-b]pyridazin-4-yl]-4-methyl-2-oxopyrrolidine-3-carbonitrile;
(3R,4S)-3-cyclopropyl-4-methyl-2-oxo-1-[6-(4,5,6,7-tetrahydropyrazolo[1,5-a]pyridin-3-yl)pyrrolo[1,2-b]pyridazin-4-yl]pyrrolidine-3-carbonitrile;
(3R,4S)-3-cyclopropyl-1-[6-(6-ethylpyridin-3-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-(4-ethylpyridin-3-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-1-[6-[6-(difluoromethoxy)pyridin-3-yl]pyrrolo[1,2-b]pyridazin-4-yl]-4-methyl-2-oxopyrrolidine-3-carbonitrile;
(3R,4S)-3-cyclopropyl-1-[6-[5-(difluoromethoxy)pyridin-3-yl]pyrrolo[1,2-b]pyridazin-4-yl]-4-methyl-2-oxopyrrolidine-3-carbonitrile;
(3R,4S)-3-cyclopropyl-1-[6-(2-ethylpyridin-3-yl)pyrrolo[1,2-b]pyridazin-4-yl]-4-methyl-2-oxopyrrolidine-3-carbonitrile;2,2,2-trifluoroacetic acid;
(3R,4S)-3-cyclopropyl-4-methyl-2-oxo-1-[6-[4-(trifluoromethyl)pyridin-3-yl]pyrrolo[1,2-b]pyridazin-4-yl]pyrrolidine-3-carbonitrile;
(3R,4S)-3-cyclopropyl-4-methyl-1-[6-(1-methylpyrazol-4-yl)pyrrolo[2,1-f][1,2,4]triazin-4-yl]-2-oxopyrrolidine-3-carbonitrile;
(3R,4S)-3-cyclopropyl-4-methyl-1-[2-(1-methylpyrazol-4-yl)-3H-imidazo[4,5-b]pyridin-7-yl]-2-oxopyrrolidine-3-carbonitrile;
(3R,4S)-3-cyclopropyl-4-methyl-1-[2-(1-methylpyrazol-4-yl)imidazo[1,2-b]pyridazin-8-yl]-2-oxopyrrolidine-3-carbonitrile;
(3R,4S)-3-cyclopropyl-1-[3-fluoro-2-(1-methylpyrazol-4-yl)imidazo[1,2-b]pyridazin-8-yl]-4-methyl-2-oxopyrrolidine-3-carbonitrile;
(3R,4S)-3-cyclopropyl-4-methyl-1-[6-(1-methylpyrazol-4-yl)pyrazolo[1,5-a]pyrazin-4-yl]-2-oxopyrrolidine-3-carbonitrile;
(3R,4S)-3-cyclopropyl-1-[6-[1-(difluoromethyl)pyrazol-4-yl]pyrazolo[1,5-a]pyrazin-4-yl]-4-methyl-2-oxopyrrolidine-3-carbonitrile;
(3R,4S)-3-cyclopropyl-4-methyl-1-[3-methyl-6-(1-methylpyrazol-4-yl)pyrazolo[1,5-a]pyrazin-4-yl]-2-oxopyrrolidine-3-carbonitrile;
(3R,4S)-3-cyclopropyl-1-[3-fluoro-6-(1-methylpyrazol-4-yl)pyrazolo[1,5-a]pyrazin-4-yl]-4-methyl-2-oxopyrrolidine-3-carbonitrile;
(3R,4S)-3-cyclopropyl-1-[6-(1-ethylpyrazol-4-yl)-3-fluoropyrazolo[1,5-a]pyrazin-4-yl]-4-methyl-2-oxopyrrolidine-3-carbonitrile;
(3R,4S)-3-cyclopropyl-1-[6-[1-(difluoromethyl)pyrazol-4-yl]-3-fluoropyrazolo[1,5-a]pyrazin-4-yl]-4-methyl-2-oxopyrrolidine-3-carbonitrile;
(3R,4S)-3-cyclopropyl-1-[3-fluoro-6-(1-propan-2-ylpyrazol-4-yl)pyrazolo[1,5-a]pyrazin-4-yl]-4-methyl-2-oxopyrrolidine-3-carbonitrile;
(3R,4S)-3-cyclopropyl-1-[3-fluoro-6-[1-[(3RS)-oxolan-3-yl]pyrazol-4-yl]pyrazolo[1,5-a]pyrazin-4-yl]-4-methyl-2-oxopyrrolidine-3-carbonitrile;
(3R,4S)-3-cyclopropyl-1-[6-(6,7-dihydro-4H-pyrazolo[5,1-c][1,4]oxazin-3-yl)-3-fluoropyrazolo[1,5-a]pyrazin-4-yl]-4-methyl-2-oxopyrrolidine-3-carbonitrile;
(3R,4S)-3-cyclopropyl-1-[6-(6-methoxypyridin-2-yl)pyrazolo[1,5-a]pyrazin-4-yl]-4-methyl-2-oxopyrrolidine-3-carbonitrile;
(3R,4S)-3-cyclopropyl-4-methyl-2-oxo-1-(6-pyridin-3-ylpyrazolo[1,5-a]pyrazin-4-yl)pyrrolidine-3-carbonitrile;
5-[4-[(3R,4S)-3-cyano-3-cyclopropyl-4-methyl-2-oxopyrrolidin-1-yl]pyrazolo[1,5-a]pyrazin-6-yl]pyridine-2-carbonitrile;2,2,2-trifluoroacetic acid;
(3R,4S)-3-cyclopropyl-1-[6-(5-fluoro-2-methoxypyridin-4-yl)pyrazolo[1,5-a]pyrazin-4-yl]-4-methyl-2-oxopyrrolidine-3-carbonitrile;
4-[4-[(3R,4S)-3-cyano-3-cyclopropyl-4-methyl-2-oxopyrrolidin-1-yl]pyrazolo[1,5-a]pyrazin-6-yl]pyridine-2-carbonitrile;
(3R,4S)-3-cyclopropyl-1-[6-[1-[(1RS)-2,2-difluorocyclopropyl]pyrazol-4-yl]pyrazolo[1,5-a]pyrazin-4-yl]-4-methyl-2-oxopyrrolidine-3-carbonitrile;
(3R,4S)-3-cyclopropyl-1-[6-(1,5-dimethylpyrazol-4-yl)pyrazolo[1,5-a]pyrazin-4-yl]-4-methyl-2-oxopyrrolidine-3-carbonitrile;
(3R,4S)-3-cyclopropyl-4-methyl-2-oxo-1-[6-(1,3,5-trimethylpyrazol-4-yl)pyrazolo[1,5-a]pyrazin-4-yl]pyrrolidine-3-carbonitrile;
(3R,4S)-3-cyclopropyl-4-methyl-2-oxo-1-[6-[1-(trideuteriomethyl)pyrazol-4-yl]pyrazolo[1,5-a]pyrazin-4-yl]pyrrolidine-3-carbonitrile;
(3R,4S)-3-cyclopropyl-1-[6-(1-ethylpyrazol-4-yl)pyrazolo[1,5-a]pyrazin-4-yl]-4-methyl-2-oxopyrrolidine-3-carbonitrile;
(3R,4S)-3-cyclopropyl-4-methyl-1-[6-[1-(oxan-4-yl)pyrazol-4-yl]pyrazolo[1,5-a]pyrazin-4-yl]-2-oxopyrrolidine-3-carbonitrile;
(3R,4S)-3-cyclopropyl-4-methyl-2-oxo-1-[6-(7-oxo-6,8-dihydro-5H-1,8-naphthyridin-3-yl)pyrazolo[1,5-a]pyrazin-4-yl]pyrrolidine-3-carbonitrile;
(3R,4S)-3-cyclopropyl-4-methyl-2-oxo-1-[6-(2-oxo-1,3-dihydropyrrolo[2,3-b]pyridin-5-yl)pyrazolo[1,5-a]pyrazin-4-yl]pyrrolidine-3-carbonitrile;
(3R,4S)-1-[3-chloro-6-(1-methylpyrazol-4-yl)pyrazolo[1,5-a]pyrazin-4-yl]-3-cyclopropyl-4-methyl-2-oxopyrrolidine-3-carbonitrile;
(3R,4S)-1-[6-[1-(1-bicyclo[1.1.1]pentanyl)pyrazol-4-yl]-3-fluoropyrazolo[1,5-a]pyrazin-4-yl]-3-cyclopropyl-4-methyl-2-oxopyrrolidine-3-carbonitrile;
(3R,4S)-3-cyclopropyl-1-[6-(1,3-dimethylpyrazol-4-yl)pyrazolo[1,5-a]pyrazin-4-yl]-4-methyl-2-oxopyrrolidine-3-carbonitrile;
(3R,4S)-3-cyclopropyl-1-[6-(2,4-dimethyl-1,3-thiazol-5-yl)pyrazolo[1,5-a]pyrazin-4-yl]-4-methyl-2-oxopyrrolidine-3-carbonitrile;
(3R,4S)-3-cyclopropyl-1-[6-[1-(difluoromethyl)-6-oxopyridin-3-yl]pyrazolo[1,5-a]pyrazin-4-yl]-4-methyl-2-oxopyrrolidine-3-carbonitrile;
(3R,4S)-3-cyclopropyl-1-[3-fluoro-6-[1-(trideuteriomethyl)pyrazol-4-yl]pyrazolo[1,5-a]pyrazin-4-yl]-4-methyl-2-oxopyrrolidine-3-carbonitrile;
(3R,4S)-3-cyclopropyl-1-[6-(3-fluoropyridin-2-yl)pyrazolo[1,5-a]pyrazin-4-yl]-4-methyl-2-oxopyrrolidine-3-carbonitrile;
(3R,4S)-3-cyclopropyl-1-[6-(5-methoxypyridin-2-yl)pyrazolo[1,5-a]pyrazin-4-yl]-4-methyl-2-oxopyrrolidine-3-carbonitrile;
(3R,4S)-3-cyclopropyl-4-methyl-1-[6-(2-methyl-1,3-thiazol-4-yl)pyrazolo[1,5-a]pyrazin-4-yl]-2-oxopyrrolidine-3-carbonitrile;
(3R,4S)-3-cyclopropyl-1-[6-(2,3-dihydro-1H-pyrrolo[2,3-b]pyridin-5-yl)pyrazolo[1,5-a]pyrazin-4-yl]-4-methyl-2-oxopyrrolidine-3-carbonitrile;
(3R,4S)-3-cyclopropyl-4-methyl-1-[2-(1-methylpyrazol-4-yl)pyrazolo[1,5-a]pyrazin-4-yl]-2-oxopyrrolidine-3-carbonitrile;
(3R,4S)-1-[6-(1-cyclopentylpyrazol-4-yl)-3-fluoropyrazolo[1,5-a]pyrazin-4-yl]-3-cyclopropyl-4-methyl-2-oxopyrrolidine-3-carbonitrile;
(3R,4S)-3-cyclopropyl-1-[3-fluoro-6-[1-(oxan-4-yl)pyrazol-4-yl]pyrazolo[1,5-a]pyrazin-4-yl]-4-methyl-2-oxopyrrolidine-3-carbonitrile;
(3R,4S)-1-[6-(1-cyclohexylpyrazol-4-yl)-3-fluoropyrazolo[1,5-a]pyrazin-4-yl]-3-cyclopropyl-4-methyl-2-oxopyrrolidine-3-carbonitrile;
(3R,4S)-1-[6-[1-(cyanomethyl)pyrazol-4-yl]-3-fluoropyrazolo[1,5-a]pyrazin-4-yl]-3-cyclopropyl-4-methyl-2-oxopyrrolidine-3-carbonitrile;
(3R,4S)-3-cyclopropyl-1-[6-(1,5-dimethylpyrazol-4-yl)-3-fluoropyrazolo[1,5-a]pyrazin-4-yl]-4-methyl-2-oxopyrrolidine-3-carbonitrile;
(3R,4S)-3-cyclopropyl-1-(3-fluoro-6-pyridin-3-ylpyrazolo[1,5-a]pyrazin-4-yl)-4-methyl-2-oxopyrrolidine-3-carbonitrile;
(3R,4S)-3-cyclopropyl-1-[3-fluoro-6-(5-methoxypyridin-3-yl)pyrazolo[1,5-a]pyrazin-4-yl]-4-methyl-2-oxopyrrolidine-3-carbonitrile;
(3R,4S)-3-cyclopropyl-1-[6-(6-methoxypyridin-3-yl)pyrazolo[1,5-a]pyrazin-4-yl]-4-methyl-2-oxopyrrolidine-3-carbonitrile;
(3R,4S)-3-cyclopropyl-4-methyl-1-[6-(1-methylindazol-5-yl)pyrazolo[1,5-a]pyrazin-4-yl]-2-oxopyrrolidine-3-carbonitrile;2,2,2-trifluoroacetic acid;
(3R,4S)-3-cyclopropyl-1-[6-(2-fluoropyridin-3-yl)pyrazolo[1,5-a]pyrazin-4-yl]-4-methyl-2-oxopyrrolidine-3-carbonitrile;2,2,2-trifluoroacetic acid;
5-[4-[(3R,4S)-3-cyano-3-cyclopropyl-4-methyl-2-oxopyrrolidin-1-yl]pyrazolo[1,5-a]pyrazin-6-yl]-N-methylpyridine-2-carboxamide;2,2,2-trifluoroacetic acid;
N-[4-[4-[(3R,4S)-3-cyano-3-cyclopropyl-4-methyl-2-oxopyrrolidin-1-yl]pyrazolo[1,5-a]pyrazin-6-yl]pyridin-2-yl]acetamide;
(3R,4S)-3-cyclopropyl-4-methyl-1-[6-(1-methylpyrazol-3-yl)pyrazolo[1,5-a]pyrazin-4-yl]-2-oxopyrrolidine-3-carbonitrile;2,2,2-trifluoroacetic acid;
(3R,4S)-3-cyclopropyl-4-methyl-2-oxo-1-[6-(1H-pyrrolo[2,3-b]pyridin-5-yl)pyrazolo[1,5-a]pyrazin-4-yl]pyrrolidine-3-carbonitrile;
(3R,4S)-3-cyclopropyl-1-[6-(2-methoxypyridin-4-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-2-oxo-1-[6-(1H-pyrazol-3-yl)pyrazolo[1,5-a]pyrazin-4-yl]pyrrolidine-3-carbonitrile;2,2,2-trifluoroacetic acid;
(3R,4S)-3-cyclopropyl-4-methyl-1-[6-(6-methylpyridin-3-yl)pyrazolo[1,5-a]pyrazin-4-yl]-2-oxopyrrolidine-3-carbonitrile;2,2,2-trifluoroacetic acid;
(3R,4S)-3-cyclopropyl-4-methyl-2-oxo-1-[6-[2-(trifluoromethyl)pyridin-4-yl]pyrazolo[1,5-a]pyrazin-4-yl]pyrrolidine-3-carbonitrile;2,2,2-trifluoroacetic acid;
(3R,4S)-3-cyclopropyl-4-methyl-1-[6-(4-methyl-3-oxopyrido[3,2-b][1,4]oxazin-7-yl)pyrazolo[1,5-a]pyrazin-4-yl]-2-oxopyrrolidine-3-carbonitrile;2,2,2-trifluoroacetic acid;
(3R,4S)-3-cyclopropyl-4-methyl-1-[6-(5-methylpyridin-3-yl)pyrazolo[1,5-a]pyrazin-4-yl]-2-oxopyrrolidine-3-carbonitrile;
(3R,4S)-3-cyclopropyl-1-[6-(2-fluoropyridin-4-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-2-oxo-1-[6-[6-(trifluoromethyl)pyridin-3-yl]pyrazolo[1,5-a]pyrazin-4-yl]pyrrolidine-3-carbonitrile;2,2,2-trifluoroacetic acid;
(3R,4S)-3-cyclopropyl-4-methyl-1-[6-(2-methyl-1,3-thiazol-5-yl)pyrazolo[1,5-a]pyrazin-4-yl]-2-oxopyrrolidine-3-carbonitrile;
(3R,4S)-3-cyclopropyl-1-[6-(2-methoxypyrimidin-5-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-1-[6-(2-methylpyridin-4-yl)pyrazolo[1,5-a]pyrazin-4-yl]-2-oxopyrrolidine-3-carbonitrile;2,2,2-trifluoroacetic acid;
(3R,4S)-3-cyclopropyl-4-methyl-1-[6-(1-methylindazol-7-yl)pyrazolo[1,5-a]pyrazin-4-yl]-2-oxopyrrolidine-3-carbonitrile;
(3R,4S)-3-cyclopropyl-1-[6-(6-fluoropyridin-3-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-1-[6-(5-methoxypyridin-3-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-1-[6-(1-methyl-6-oxopyridin-3-yl)pyrazolo[1,5-a]pyrazin-4-yl]-2-oxopyrrolidine-3-carbonitrile;
(3R,4S)-3-cyclopropyl-1-[6-(5-fluoropyridin-3-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-1-[6-(1H-imidazol-2-yl)pyrazolo[1,5-a]pyrazin-4-yl]-4-methyl-2-oxopyrrolidine-3-carbonitrile;2,2,2-trifluoroacetic acid;
(3R,4S)-1-[6-(5-chloropyridin-3-yl)pyrazolo[1,5-a]pyrazin-4-yl]-3-cyclopropyl-4-methyl-2-oxopyrrolidine-3-carbonitrile;2,2,2-trifluoroacetic acid;
N-[5-[4-[(3R,4S)-3-cyano-3-cyclopropyl-4-methyl-2-oxopyrrolidin-1-yl]pyrazolo[1,5-a]pyrazin-6-yl]pyridin-3-yl]acetamide;2,2,2-trifluoroacetic acid;
(3R,4S)-3-cyclopropyl-1-[6-(2-methoxypyridin-3-yl)pyrazolo[1,5-a]pyrazin-4-yl]-4-methyl-2-oxopyrrolidine-3-carbonitrile;
(3R,4S)-3-cyclopropyl-4-methyl-1-[6-[1-(oxetan-3-yl)pyrazol-4-yl]pyrazolo[1,5-a]pyrazin-4-yl]-2-oxopyrrolidine-3-carbonitrile;
4-[4-[(3R,4S)-3-cyano-3-cyclopropyl-4-methyl-2-oxopyrrolidin-1-yl]pyrazolo[1,5-a]pyrazin-6-yl]-N-methylpyridine-2-carboxamide;2,2,2-trifluoroacetic acid;
(3R,4S)-3-cyclopropyl-1-[6-(3,6-dimethoxypyridazin-4-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-1-[6-(2-methylpyrazol-3-yl)pyrazolo[1,5-a]pyrazin-4-yl]-2-oxopyrrolidine-3-carbonitrile;2,2,2-trifluoroacetic acid;
(3R,4S)-3-cyclopropyl-1-[6-(6-fluoropyridin-2-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-1-[6-[2-(difluoromethoxy)pyridin-3-yl]pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-1-[6-(6-methylsulfonylpyridin-3-yl)pyrazolo[1,5-a]pyrazin-4-yl]-2-oxopyrrolidine-3-carbonitrile;2,2,2-trifluoroacetic acid;
(3R,4S)-3-cyclopropyl-1-[6-(5-hydroxypyridin-3-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-1-[6-(1-methylindazol-6-yl)pyrazolo[1,5-a]pyrazin-4-yl]-2-oxopyrrolidine-3-carbonitrile;2,2,2-trifluoroacetic acid;
(3R,4S)-3-cyclopropyl-4-methyl-2-oxo-1-[6-[5-(trifluoromethyl)pyridin-3-yl]pyrazolo[1,5-a]pyrazin-4-yl]pyrrolidine-3-carbonitrile;2,2,2-trifluoroacetic acid;
(3R,4S)-3-cyclopropyl-4-methyl-1-[6-(1-methyl-2-oxopyridin-4-yl)pyrazolo[1,5-a]pyrazin-4-yl]-2-oxopyrrolidine-3-carbonitrile;2,2,2-trifluoroacetic acid;
5-[4-[(3R,4S)-3-cyano-3-cyclopropyl-4-methyl-2-oxopyrrolidin-1-yl]pyrazolo[1,5-a]pyrazin-6-yl]-2-methoxypyridine-3-carbonitrile;2,2,2-trifluoroacetic acid;
(3R,4S)-3-cyclopropyl-1-(6-imidazo[1,2-a]pyridin-6-ylpyrazolo[1,5-a]pyrazin-4-yl)-4-methyl-2-oxopyrrolidine-3-carbonitrile;2,2,2-trifluoroacetic acid;
(3R,4S)-3-cyclopropyl-1-[6-[2-(dimethylamino)pyrimidin-5-yl]pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-2-oxo-1-(6-pyrimidin-5-ylpyrazolo[1,5-a]pyrazin-4-yl)pyrrolidine-3-carbonitrile;2,2,2-trifluoroacetic acid;
tert-butyl N-[5-[4-[(3R,4S)-3-cyano-3-cyclopropyl-4-methyl-2-oxopyrrolidin-1-yl]pyrazolo[1,5-a]pyrazin-6-yl]pyridin-2-yl]-N-methylcarbamate;2,2,2-trifluoroacetic acid;
(3R,4S)-3-cyclopropyl-1-[6-(6-hydroxypyridin-3-yl)pyrazolo[1,5-a]pyrazin-4-yl]-4-methyl-2-oxopyrrolidine-3-carbonitrile;2,2,2-trifluoroacetic acid;
(3R,4S)-3-cyclopropyl-4-methyl-1-[2-(1-methylpyrazol-4-yl)furo[3,2-b]pyridin-7-yl]-2-oxopyrrolidine-3-carbonitrile;
(3R,4S)-3-cyclopropyl-4-methyl-1-[7-(1-methylpyrazol-4-yl)imidazo[1,2-c]pyrimidin-5-yl]-2-oxopyrrolidine-3-carbonitrile;
(3R,4S)-3-cyclopropyl-1-[6-[1-(difluoromethyl)pyrazol-4-yl]-3-fluoropyrazolo[1,5-a]pyridin-4-yl]-4-methyl-2-oxopyrrolidine-3-carbonitrile;
(3R,4S)-3-cyclopropyl-4-methyl-1-[6-(1-methylpyrazol-4-yl)pyrazolo[1,5-a]pyridin-4-yl]-2-oxopyrrolidine-3-carbonitrile;
(3R,4S)-3-cyclopropyl-4-methyl-1-[2-(1-methylpyrazol-4-yl)pyrazolo[1,5-a]pyridin-4-yl]-2-oxopyrrolidine-3-carbonitrile;
(3R,4S)-3-cyclopropyl-4-methyl-1-[2-(1-methylpyrazol-4-yl)pyrazolo[1,5-a]pyrimidin-7-yl]-2-oxopyrrolidine-3-carbonitrile;
(3R,4S)-3-cyclopropyl-4-methyl-1-[5-(1-methylpyrazol-4-yl)-1H-pyrazolo[3,4-b]pyridin-3-yl]-2-oxopyrrolidine-3-carbonitrile;
(3R,4S)-3-cyclopropyl-4-methyl-1-[1-(1-methylpyrazol-4-yl)pyrazolo[3,4-c]pyridin-3-yl]-2-oxopyrrolidine-3-carbonitrile;
(3R,4S)-3-cyclopropyl-1-[1-[1-(difluoromethyl)pyrazol-4-yl]pyrazolo[3,4-c]pyridin-3-yl]-4-methyl-2-oxopyrrolidine-3-carbonitrile;
(3R,4S)-3-cyclopropyl-1-[1-(1-ethylpyrazol-4-yl)pyrazolo[3,4-c]pyridin-3-yl]-4-methyl-2-oxopyrrolidine-3-carbonitrile;
(3R,4S)-3-cyclopropyl-4-methyl-1-[1-[1-(oxetan-3-yl)pyrazol-4-yl]pyrazolo[3,4-c]pyridin-3-yl]-2-oxopyrrolidine-3-carbonitrile;
(3R,4S)-3-cyclopropyl-4-methyl-1-[1-(1-methyl-6-oxopyridin-2-yl)pyrazolo[3,4-c]pyridin-3-yl]-2-oxopyrrolidine-3-carbonitrile;
(3R,4S)-3-cyclopropyl-4-methyl-2-oxo-1-[1-(1-propan-2-ylpyrazol-4-yl)pyrazolo[3,4-c]pyridin-3-yl]pyrrolidine-3-carbonitrile;
(3R,4S)-3-cyclopropyl-4-methyl-1-[1-(2-methylpyridin-4-yl)pyrazolo[3,4-c]pyridin-3-yl]-2-oxopyrrolidine-3-carbonitrile;
(3R,4S)-3-cyclopropyl-4-methyl-1-[1-(6-methylpyridin-3-yl)pyrazolo[3,4-c]pyridin-3-yl]-2-oxopyrrolidine-3-carbonitrile;
(3R,4S)-3-cyclopropyl-4-methyl-1-[3-(1-methylpyrazol-4-yl)pyrrolo[1,2-a]pyrimidin-6-yl]-2-oxopyrrolidine-3-carbonitrile;
(3R,4S)-3-cyclopropyl-4-methyl-1-[6-(1-methylpyrazol-4-yl)-[1,3]thiazolo[5,4-c]pyridin-4-yl]-2-oxopyrrolidine-3-carbonitrile;
(3R,4S)-3-cyclopropyl-4-methyl-1-[7-(1-methylpyrazol-4-yl)-[1,2,4]triazolo[1,5-a]pyridin-5-yl]-2-oxopyrrolidine-3-carbonitrile;
(3R,4S)-3-cyclopropyl-4-methyl-1-[6-(1-methylpyrazol-4-yl)thieno[3,2-d]pyrimidin-4-yl]-2-oxopyrrolidine-3-carbonitrile;
(3R,4S)-3-cyclopropyl-4-methyl-1-[7-(1-methylpyrazol-4-yl)imidazo[1,2-a]pyridin-5-yl]-2-oxopyrrolidine-3-carbonitrile;
(3R,4S)-3-cyclopropyl-4-methyl-1-[5-(1-methylpyrazol-4-yl)-1H-pyrrolo[2,3-b]pyridin-3-yl]-2-oxopyrrolidine-3-carbonitrile;
(3R,4S)-3-cyclopropyl-4-methyl-1-[6-[1-(2-oxaspiro[3.3]heptan-6-yl)pyrazol-4-yl]pyrazolo[1,5-a]pyrazin-4-yl]-2-oxopyrrolidine-3-carbonitrile;
(3R,4S)-3-cyclopropyl-1-[6-[1-(3,3-difluorocyclobutyl)pyrazol-4-yl]pyrazolo[1,5-a]pyrazin-4-yl]-4-methyl-2-oxopyrrolidine-3-carbonitrile;
(3R,4S)-3-cyclopropyl-1-[3-fluoro-6-[1-(oxetan-3-yl)pyrazol-4-yl]pyrazolo[1,5-a]pyrazin-4-yl]-4-methyl-2-oxopyrrolidine-3-carbonitrile;
(3R,4S)-1-[6-[1-(3-cyanocyclobutyl)pyrazol-4-yl]-3-fluoropyrazolo[1,5-a]pyrazin-4-yl]-3-cyclopropyl-4-methyl-2-oxopyrrolidine-3-carbonitrile;
(3R,4S)-1-[6-[1-(3-cyanocyclobutyl)pyrazol-4-yl]-3-fluoropyrazolo[1,5-a]pyrazin-4-yl]-3-cyclopropyl-4-methyl-2-oxopyrrolidine-3-carbonitrile;
(3R,4S)-3-cyclopropyl-4-methyl-2-oxo-1-[6-(1-pyridin-3-ylpyrazol-4-yl)pyrazolo[1,5-a]pyrazin-4-yl]pyrrolidine-3-carbonitrile;
(3R,4S)-3-cyclopropyl-4-methyl-2-oxo-1-[6-(1-pyridin-4-ylpyrazol-4-yl)pyrazolo[1,5-a]pyrazin-4-yl]pyrrolidine-3-carbonitrile;
(3R,4S)-3-cyclopropyl-4-methyl-2-oxo-1-[6-(1-pyridin-2-ylpyrazol-4-yl)pyrazolo[1,5-a]pyrazin-4-yl]pyrrolidine-3-carbonitrile;
(3R,4S)-1-[6-[1-(3-cyano-1-bicyclo[1.1.1]pentanyl)pyrazol-4-yl]-3-fluoropyrazolo[1,5-a]pyrazin-4-yl]-3-cyclopropyl-4-methyl-2-oxopyrrolidine-3-carbonitrile;
(3R,4S)-3-cyclopropyl-1-[3-fluoro-6-[1-(2-oxaspiro[3.3]heptan-6-yl)pyrazol-4-yl]pyrazolo[1,5-a]pyrazin-4-yl]-4-methyl-2-oxopyrrolidine-3-carbonitrile;
(3R,4S)-3-cyclopropyl-1-[3-fluoro-6-[1-(3-methoxycyclobutyl)pyrazol-4-yl]pyrazolo[1,5-a]pyrazin-4-yl]-4-methyl-2-oxopyrrolidine-3-carbonitrile;
(3R,4S)-3-cyclopropyl-1-[3-fluoro-6-[1-(3-methyloxetan-3-yl)pyrazol-4-yl]pyrazolo[1,5-a]pyrazin-4-yl]-4-methyl-2-oxopyrrolidine-3-carbonitrile;
(3R,4S)-3-cyclopropyl-1-[3-fluoro-6-[1-(3-hydroxy-3-methylcyclobutyl)pyrazol-4-yl]pyrazolo[1,5-a]pyrazin-4-yl]-4-methyl-2-oxopyrrolidine-3-carbonitrile;
(3R,4S)-1-[6-[1-[(1-cyanocyclopropyl)methyl]pyrazol-4-yl]-3-fluoropyrazolo[1,5-a]pyrazin-4-yl]-3-cyclopropyl-4-methyl-2-oxopyrrolidine-3-carbonitrile;
(3R,4S)-3-cyclopropyl-1-[3-fluoro-6-[1-[(3-methyloxetan-3-yl)methyl]pyrazol-4-yl]pyrazolo[1,5-a]pyrazin-4-yl]-4-methyl-2-oxopyrrolidine-3-carbonitrile;
(3R,4S)-3-cyclopropyl-1-[3-fluoro-6-[1-(oxetan-3-ylmethyl)pyrazol-4-yl]pyrazolo[1,5-a]pyrazin-4-yl]-4-methyl-2-oxopyrrolidine-3-carbonitrile;
(3R,4S)-3-cyclopropyl-1-[3-fluoro-6-[1-[(2RS,3RS)-2-methyloxetan-3-yl]pyrazol-4-yl]pyrazolo[1,5-a]pyrazin-4-yl]-4-methyl-2-oxopyrrolidine-3-carbonitrile;
(3R,4S)-3-cyclopropyl-1-[3-fluoro-6-[1-[(2RS,3 SR)-2-methyloxetan-3-yl]pyrazol-4-yl]pyrazolo[1,5-a]pyrazin-4-yl]-4-methyl-2-oxopyrrolidine-3-carbonitrile;
(3R,4S)-1-[6-[1-(3-cyano-3-methylcyclobutyl)pyrazol-4-yl]-3-fluoropyrazolo[1,5-a]pyrazin-4-yl]-3-cyclopropyl-4-methyl-2-oxopyrrolidine-3-carbonitrile;
(3R,4S)-1-[6-[1-(3-cyano-3-methylcyclobutyl)pyrazol-4-yl]-3-fluoropyrazolo[1,5-a]pyrazin-4-yl]-3-cyclopropyl-4-methyl-2-oxopyrrolidine-3-carbonitrile;
(3R,4S)-1-[6-[1-(3-cyano-3-fluorocyclobutyl)pyrazol-4-yl]-3-fluoropyrazolo[1,5-a]pyrazin-4-yl]-3-cyclopropyl-4-methyl-2-oxopyrrolidine-3-carbonitrile;
(3R,4S)-1-[3-fluoro-6-(1-methylpyrazol-4-yl)pyrazolo[1,5-a]pyrazin-4-yl]-4-methyl-2-oxo-3-propan-2-ylpyrrolidine-3-carbonitrile;
(3R,4S)-3-cyclopropyl-1-[3-fluoro-6-[1-(1-methylazetidin-3-yl)pyrazol-4-yl]pyrazolo[1,5-a]pyrazin-4-yl]-4-methyl-2-oxopyrrolidine-3-carbonitrile;
(3R,4S)-3-cyclopropyl-4-methyl-1-[6-[1-(1-methylpiperidin-4-yl)pyrazol-4-yl]pyrazolo[1,5-a]pyrazin-4-yl]-2-oxopyrrolidine-3-carbonitrile;
(3R,4S)-3-cyclopropyl-4-methyl-1-[6-[1-[(3RS)-1-methylpyrrolidin-3-yl]pyrazol-4-yl]pyrazolo[1,5-a]pyrazin-4-yl]-2-oxopyrrolidine-3-carbonitrile;
(3R,4S)-3-cyclopropyl-4-methyl-1-[6-[1-(2-methyl-2-azaspiro[3.3]heptan-6-yl)pyrazol-4-yl]pyrazolo[1,5-a]pyrazin-4-yl]-2-oxopyrrolidine-3-carbonitrile;
(3R,4S)-3-cyclopropyl-4-methyl-1-[6-[1-(2-methyl-2-azaspiro[3.3]heptan-6-yl)pyrazol-4-yl]pyrrolo[1,2-b]pyridazin-4-yl]-2-oxopyrrolidine-3-carbonitrile;
(3R,4S)-3-cyclopropyl-4-methyl-1-[6-[1-(1-methylazetidin-3-yl)pyrazol-4-yl]pyrrolo[1,2-b]pyridazin-4-yl]-2-oxopyrrolidine-3-carbonitrile;
(3R,4S)-3-cyclopropyl-4-methyl-1-[6-[4-[(2RS)-4-methylmorpholin-2-yl]pyrazol-1-yl]pyrrolo[1,2-b]pyridazin-4-yl]-2-oxopyrrolidine-3-carbonitrile;
(3R,4S)-3-cyclopropyl-4-methyl-1-[6-(5-methyl-1H-pyrazol-3-yl)pyrazolo[1,5-a]pyridin-4-yl]-2-oxopyrrolidine-3-carbonitrile;
(3R,4S)-3-cyclopropyl-4-methyl-1-[6-[1-(2-oxaspiro[3.3]heptan-6-yl)pyrazol-4-yl]pyrrolo[1,2-b]pyridazin-4-yl]-2-oxopyrrolidine-3-carbonitrile;
(3R,4S)-1-[6-[1-(3-cyano-1-bicyclo[1.1.1]pentanyl)pyrazol-4-yl]pyrrolo[1,2-b]pyridazin-4-yl]-3-cyclopropyl-4-methyl-2-oxopyrrolidine-3-carbonitrile;
(3R,4S)-3-cyclopropyl-1-[6-[3-fluoro-1-(oxetan-3-yl)pyrazol-4-yl]pyrrolo[1,2-b]pyridazin-4-yl]-4-methyl-2-oxopyrrolidine-3-carbonitrile;
(3R,4S)-3-cyclopropyl-1-[6-[1-[1-(hydroxymethyl)cyclopropyl]pyrazol-4-yl]pyrrolo[1,2-b]pyridazin-4-yl]-4-methyl-2-oxopyrrolidine-3-carbonitrile;
(3R,4S)-1-[6-[1-[1-(cyanomethyl)cyclopropyl]pyrazol-4-yl]pyrrolo[1,2-b]pyridazin-4-yl]-3-cyclopropyl-4-methyl-2-oxopyrrolidine-3-carbonitrile;
(3R,4S)-1-[6-[1-[(1-cyanocyclopropyl)methyl]pyrazol-4-yl]pyrrolo[1,2-b]pyridazin-4-yl]-3-cyclopropyl-4-methyl-2-oxopyrrolidine-3-carbonitrile;
(3R,4S)-3-cyclopropyl-4-methyl-1-[6-[1-[(3-methyloxetan-3-yl)methyl]pyrazol-4-yl]pyrrolo[1,2-b]pyridazin-4-yl]-2-oxopyrrolidine-3-carbonitrile;
(3R,4S)-1-[6-[1-(3-cyano-3-fluorocyclobutyl)pyrazol-4-yl]pyrrolo[1,2-b]pyridazin-4-yl]-3-cyclopropyl-4-methyl-2-oxopyrrolidine-3-carbonitrile;
(3R,4S)-3-cyclopropyl-1-[6-[1-[(3R*)-5,5-difluorooxan-3-yl]pyrazol-4-yl]pyrrolo[1,2-b]pyridazin-4-yl]-4-methyl-2-oxopyrrolidine-3-carbonitrile;
(3R,4S)-3-cyclopropyl-1-[6-[1-[(3R*)-5,5-difluorooxan-3-yl]pyrazol-4-yl]pyrrolo[1,2-b]pyridazin-4-yl]-4-methyl-2-oxopyrrolidine-3-carbonitrile;
(3R,4S)-1-[6-[1-(3-cyanocyclobutyl)pyrazol-4-yl]pyrrolo[1,2-b]pyridazin-4-yl]-3-cyclopropyl-4-methyl-2-oxopyrrolidine-3-carbonitrile;
(3R,4S)-1-[6-[1-(3-cyanocyclobutyl)pyrazol-4-yl]pyrrolo[1,2-b]pyridazin-4-yl]-3-cyclopropyl-4-methyl-2-oxopyrrolidine-3-carbonitrile;
(3R,4S)-3-cyclopropyl-4-methyl-1-[6-[1-(1-methylpiperidin-4-yl)pyrazol-4-yl]pyrrolo[1,2-b]pyridazin-4-yl]-2-oxopyrrolidine-3-carbonitrile;
(3R,4S)-3-cyclopropyl-4-methyl-1-[6-[1-[(3RS)-1-methylpyrrolidin-3-yl]pyrazol-4-yl]pyrrolo[1,2-b]pyridazin-4-yl]-2-oxopyrrolidine-3-carbonitrile;
(3R,4S)-3-cyclopropyl-4-methyl-1-[6-(4-methylpyrazol-1-yl)pyrrolo[1,2-b]pyridazin-4-yl]-2-oxopyrrolidine-3-carbonitrile;
(3R,4S)-3-cyclopropyl-4-methyl-1-[6-(3-methylpyrazol-1-yl)pyrrolo[1,2-b]pyridazin-4-yl]-2-oxopyrrolidine-3-carbonitrile;
(3R,4S)-3-cyclopropyl-4-methyl-2-oxo-1-(6-pyrazol-1-ylpyrrolo[1,2-b]pyridazin-4-yl)pyrrolidine-3-carbonitrile;
(3R,4S)-3-cyclopropyl-1-(6-imidazol-1-ylpyrrolo[1,2-b]pyridazin-4-yl)-4-methyl-2-oxopyrrolidine-3-carbonitrile;
(3R,4S)-3-cyclopropyl-1-[6-[4-(methoxymethyl)pyrazol-1-yl]pyrrolo[1,2-b]pyridazin-4-yl]-4-methyl-2-oxopyrrolidine-3-carbonitrile;
(3R,4S)-3-cyclopropyl-1-[6-[4-(difluoromethyl)pyrazol-1-yl]pyrrolo[1,2-b]pyridazin-4-yl]-4-methyl-2-oxopyrrolidine-3-carbonitrile;
(3R,4S)-1-[6-[4-(cyanomethyl)pyrazol-1-yl]pyrrolo[1,2-b]pyridazin-4-yl]-3-cyclopropyl-4-methyl-2-oxopyrrolidine-3-carbonitrile;
(3R,4S)-3-cyclopropyl-4-methyl-2-oxo-1-[6-(2-oxopyridin-1-yl)pyrrolo[1,2-b]pyridazin-4-yl]pyrrolidine-3-carbonitrile;
(3R,4S)-3-cyclopropyl-4-methyl-1-[6-(4-methylimidazol-1-yl)pyrrolo[1,2-b]pyridazin-4-yl]-2-oxopyrrolidine-3-carbonitrile;
(3R,4S)-3-cyclopropyl-4-methyl-1-[6-(3-methyl-1,2,4-triazol-1-yl)pyrrolo[1,2-b]pyridazin-4-yl]-2-oxopyrrolidine-3-carbonitrile;
(3R,4S)-3-cyclopropyl-4-methyl-1-[6-(4-methyltriazol-2-yl)pyrrolo[1,2-b]pyridazin-4-yl]-2-oxopyrrolidine-3-carbonitrile;
(3R,4S)-1-[2-[1-(1-bicyclo[1.1.1]pentanyl)pyrazol-4-yl]furo[3,2-b]pyridin-7-yl]-3-cyclopropyl-4-methyl-2-oxopyrrolidine-3-carbonitrile;
(3R,4S)-3-cyclopropyl-4-methyl-1-[2-(4-methylpyrazol-1-yl)furo[3,2-b]pyridin-7-yl]-2-oxopyrrolidine-3-carbonitrile;
(3R,4S)-3-cyclopropyl-4-methyl-2-oxo-1-[2-(1-propan-2-ylpyrazol-4-yl)furo[3,2-b]pyridin-7-yl]pyrrolidine-3-carbonitrile;
(3R,4S)-3-cyclopropyl-1-[2-(1-cyclopropylpyrazol-4-yl)furo[3,2-b]pyridin-7-yl]-4-methyl-2-oxopyrrolidine-3-carbonitrile;
(3R,4S)-3-cyclopropyl-4-methyl-1-[5-[1-(oxetan-3-yl)pyrazol-4-yl]-1,3-benzothiazol-7-yl]-2-oxopyrrolidine-3-carbonitrile;
(3R,4S)-3-cyclopropyl-1-[5-[1-(difluoromethyl)pyrazol-4-yl]-1,3-benzothiazol-7-yl]-4-methyl-2-oxopyrrolidine-3-carbonitrile;
(3R,4S)-3-cyclopropyl-4-methyl-1-[5-(1-methylpyrazol-4-yl)-1,3-benzothiazol-7-yl]-2-oxopyrrolidine-3-carbonitrile;
(3R,4S)-3-cyclopropyl-4-methyl-1-[6-(1-methylpyrazol-4-yl)-2,1-benzothiazol-4-yl]-2-oxopyrrolidine-3-carbonitrile;
(3R,4S)-3-cyclopropyl-4-methyl-1-[6-[1-(oxetan-3-yl)pyrazol-4-yl]-[1,3]thiazolo[5,4-c]pyridin-4-yl]-2-oxopyrrolidine-3-carbonitrile;
(3R,4S)-3-cyclopropyl-1-[6-[1-(difluoromethyl)pyrazol-4-yl]-[1,3]thiazolo[5,4-c]pyridin-4-yl]-4-methyl-2-oxopyrrolidine-3-carbonitrile;
(3R,4S)-3-cyclopropyl-1-[6-[1-(2,2-difluoroethyl)pyrazol-4-yl]-[1,3]thiazolo[5,4-c]pyridin-4-yl]-4-methyl-2-oxopyrrolidine-3-carbonitrile;
(3R,4S)-3-cyclopropyl-1-[6-[1-(3-methoxycyclobutyl)pyrazol-4-yl]-[1,3]thiazolo[5,4-c]pyridin-4-yl]-4-methyl-2-oxopyrrolidine-3-carbonitrile;
(3R,4S)-1-[6-[1-(3-cyanocyclobutyl)pyrazol-4-yl]-[1,3]thiazolo[5,4-c]pyridin-4-yl]-3-cyclopropyl-4-methyl-2-oxopyrrolidine-3-carbonitrile;
(3R,4S)-3-cyclopropyl-1-[6-(1-cyclopropylpyrazol-4-yl)-[1,3]thiazolo[5,4-c]pyridin-4-yl]-4-methyl-2-oxopyrrolidine-3-carbonitrile; and
(3R,4S)-3-cyclopropyl-4-methyl-1-[6-[1-(1-methylazetidin-3-yl)pyrazol-4-yl]-[1,3]thiazolo[5,4-c]pyridin-4-yl]-2-oxopyrrolidine-3-carbonitrile;
   or a pharmaceutically acceptable salt thereof.

In a thirty-fifth embodiment, the present disclosure provides a pharmaceutical composition comprising a compound of the present disclosure (e.g., according to any of the preceding embodiments), or a pharmaceutically acceptable salt thereof.

### Definitions

As used herein, the phrase “optionally substituted” is used interchangeably with the phrase “substituted or unsubstituted.” In general, the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. In some embodiments, an optionally substituted group can be substituted with one or more substituents, each of which can the same or different. In some embodiments, the “one or more” substituents can be 1, 2, 3, 4, 5, 6, etc. substituents, each of which can the same or different. In some embodiment, the “one or more” substituents can be 1 to 6, 1 to 4, 1 to 3 or 1 to 2 substituents, each of which can the same or different.

As used herein, “halogen” or “halo” may be fluorine, chlorine, bromine or iodine.

As used herein, “hydroxyl” or “hydroxy” refers to the group —OH.

As used herein, the number of carbon atoms in a group is specified herein by the prefix “Cₓ₋ₓₓ”, wherein x and xx are integers. For example, “C₁₋₄ alkyl” is an alkyl group which has from 1 to 4 carbon atoms.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “haloalkyl” refers to an alkyl group as defined herein, wherein at least one of the hydrogen atoms is replaced by a halo atom. As used herein, the term “C₁₋₄haloalkyl” refers to a C₁₋₄alkyl group as defined herein, wherein at least one of the hydrogen atoms is replaced by a halo atom. Similarly, the term “C₁₋₆halkalkyl” refers to a C₁₋₆ alkyl group as defined herein, wherein at least one of the hydrogen atoms is replaced by a halo atom. The C₁₋₄haloalkyl or C₁₋₆haloalkyl groups can be monohaloalkyl, dihaloalkyl or polyhaloalkyl including perhaloalkyl. A monohalo-C₁₋₄alkyl or monohalo-C₁₋₆alkyl can have one iodo, bromo, chloro or fluoro within the alkyl group. Dihalo-C₁₋₄alkyl, dihalo-C₁₋₆alkyl, polyhalo-C₁₋₄alkyl, and polyhalo-C₁₋₆alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Typically the polyhalo-C₁₋₆alkyl group contains up to 13, or 12, or 11, or 10, or 9, or 8, or 7, or 6, or 5, or 4, or 3, or 2 halo groups. Non-limiting examples of C₁₋₄haloalkyl or C₁₋₆haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl, dichloropropyl, difluoropentyl, and monochlorohexyl. A perhalo-C₁₋₄alkyl or perhalo-C₁₋₆alkyl group refers to a C₁₋₄alkyl or C₁₋₆alkyl group having all hydrogen atoms replaced with halo atoms.

As used herein, the term “oxo” (═O) refers to an oxygen atom connected to a carbon or sulfur atom by a double bond. Examples include carbonyl, sulfinyl, or sulfonyl groups (—C(O)—, —S(O)— or —S(O)₂—) such as, a ketone, aldehyde, or part of an acid, ester, amide, lactone, or lactam group and the like.

As used herein, the terms “aryl”, “aryl group”, “aryl ring”, “aromatic group” and “aromatic ring” are used interchangeably to refer to an aromatic 6 to 12 membered (i.e. C₆₋₁₀ aryl) monocyclic or bicyclic carbon ring system. Examples of aryl systems include, but are not limited to, phenyl, naphthyl and the like.

As used herein, the terms “heteroaryl”, “heteroaryl group”, “heteroaromatic” and “heteroaromatic ring” are used interchangeably to refer to an aromatic 5 to 10 membered monocyclic or bicyclic ring system, having at least one heteroatom (e.g., oxygen, sulfur, nitrogen, or combinations thereof), and wherein N can be oxidized (e.g., N(O)) or quaternized, and S can be optionally oxidized to sulfoxide and sulfone. In some embodiments, the heteroasyl is a 5 to 6 membered heteroaryl. “Heteroaryl” includes a heteroaromatic group that is fused to a phenyl group or non-aromatic heterocycle such as tetrahydrofuran, pyran, pyrrolidine, piperidine, and the like. Examples of heteroaryls include pyrrole, pyridyl, pyrazole, thienyl, furanyl, oxazolyl, imidazole, oxazole, isoxazole, thiazole, isothiazole, triazole, tetrazolyl, triazinyl, pyrimidyl, pyrazinyl, thiazolyl, indolyl, indazolyl, benzofuranyl, quinoxalinyl and the like. In some embodiments, heteroaryl is selected from pyrazole, imidazole, oxazole, isoxazole, thiazole, isothiazole, triazole and pyrrole.

As used herein, the term “cycloalkyl” refers to completely saturated monocyclic or bicyclic (e.g., fused, spiro or bridged) hydrocarbon groups of 3-12 carbon atoms (i.e., C₃₋₁₂ cycloalkyl), 3-6 carbon atoms (i.e., C₃₋₆ cycloalkyl), 3-8 carbon atoms (i.e., C₃₋₈ cycloalkyl), or 5-7 carbon atoms (i.e., C₅₋₇ cycloalkyl). Examples of C₃₋₈ cycloalkyl include, but are not limited to, cyclopropyl, cyclobutyl, cyclopentyl, and cyclohexyl.

As used herein, the term “heterocycloalkyl” refers to completely saturated 4 to 12 membered monocyclic or bicyclic (e.g., a bridged, fused, or spiro bicyclic) ring system, having at least one heteroatom (e.g., oxygen, sulfur, nitrogen, or combinations thereof). In some embodiments, the heterocycloalkyl group is a 4 to 10 membered heterocycloalkyl group. In some embodiments, the heterocycloalkyl group is a 4 to 6 membered monocyclic heterocycloalkyl group. In some embodiments, the heterocycloalkyl group is a 4 to 7 membered monocyclic heterocycloalkyl group. In some embodiments, the heterocycloalkyl group is a 7 to 10 membered bicyclic heterocycloalkyl group. In some embodiments, the heterocycloalkyl group is a 7 to 8 membered bicyclic heterocycloalkyl group.

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6 membered monocyclic heterocyclyl group. In some embodiments, the heterocyclyl group is a 4 to 7 membered monocyclic saturated heterocyclyl group. In some embodiments, the heterocyclyl group is a 3 to 7 membered monocyclic saturated heterocyclyl group. In some embodiments, the heterocyclyl group is a 8 to 10 membered bicyclic heterocyclyl group. In some embodiments, the heterocyclyl group is a 8 to 10 membered bicyclic saturated heterocyclyl group.

As used herein the term “spiral” ring means a two-ring system wherein both rings share one common atom. Examples of spiral rings include, 2-oxa-6-azaspiro[3.3]heptanyl and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. 5-oxabicyclo[2.1.1]hexane).

The phrase “pharmaceutically acceptable” indicates that the substance, composition or dosage form must be compatible chemically and/or toxicologically, with the other ingredients comprising a formulation, and/or the mammal being treated therewith.

Also included in the present disclosure are compounds of Formula (I-1), (I-2), (I-1), (I-2), (II-1), (II-2), (11-3), (II-4), (II-5), (11-6), (11-7), (II-8), (II-9), (11-10), (II-11), (II-12), (II-13), (II-14), (II-15), (II-16), (II-17), (III-1), (III-2), (III-3), (III-4), (III-5), (IV-1), (IV-2), (IV-3), (IV-4), or (IV-5), as well as all stereoisomers (including diastereoisomers and enantiomers), rotamers, tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts. The compounds of the present disclosure, including salts, hydrates and solvates thereof, may inherently or by design form polymorphs.

As used herein, the term “a,” “an,” “the” and similar terms used in the context of the present disclosure (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the disclosure and does not pose a limitation on the scope of the disclosure otherwise claimed.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

It will be recognized by those skilled in the art that the compounds of the present disclosure may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

Certain of the compounds described herein contain one or more asymmetric centers or axes and may thus give rise to enantiomers, diastereomers, and other stereoisomeric forms that may be defined, in terms of absolute stereochemistry, as (R)- or (S)-. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase). In some embodiment, the compounds described herein are isolated stereoisomers wherein each of the compounds has one stereocenter and the stereoisomer is in the R configuration. In other embodiment, the compounds described herein are isolated stereoisomers wherein each of the compounds has one stereocenter and the stereoisomer is in the S configuration. In one embodiment, the compounds described herein are isolated stereoisomers wherein each of the compounds has two stereocenters and the stereoisomer is in the R R configuration. In one embodiment, the compounds described herein are isolated stereoisomers wherein each of the compounds has two stereocenters and the stereoisomer is in the R S configuration. In one embodiment, the compounds described herein are isolated stereoisomers stereoisomer wherein each of the compounds has two stereocenters and the stereoisomer is in the S R configuration. In one embodiment, the compounds described herein are isolated stereoisomers stereoisomer wherein each of the compounds has two stereocenters and the stereoisomer is in the S S configuration. In one embodiment, the compounds described herein each have one or two stereocenters and are racemic mixtures.

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a particular enantiomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired enantiomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers is at least 20%, 30%, 40%, 50%, 60%, 70%, 80%, 90%, 95%, 97%, 99%, 99.5% or 99.9%. The stereoisomeric purity means the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or IPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated. All such forms are embraced within the scope of the disclosure. In addition, some compounds may exhibit polymorphism. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

Furthermore, the compounds of the present disclosure, including their salts, may also be obtained in the form of their hydrates, or include other solvents used for their crystallization. The compounds of the present disclosure may inherently or by design form solvates with pharmaceutically acceptable solvents (including water); therefore, it is intended that the disclosure embrace both solvated and unsolvated forms. The term “solvate” refers to a molecular complex of a compound of the present disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Compounds of the disclosure that contain groups capable of acting as donors and/or acceptors for hydrogen bonds may be capable of forming co-crystals with suitable co-crystal formers. These co-crystals may be prepared from the compounds by known co-crystal forming procedures. Such procedures include grinding, heating, co-subliming, co-melting, or contacting in solution the compounds with the co-crystal former under crystallization conditions and isolating co-crystals thereby formed. Suitable co-crystal formers include those described in WO 2004/078163. Hence the disclosure further provides co-crystals comprising a compound described herein.

In one embodiment, the disclosure provides deuterated compounds disclosed herein, in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance.

In another embodiment, the present disclosure is a pharmaceutical composition comprising at least on compound described herein, or a pharmaceutically acceptable salt thereof, and at least one pharmaceutically acceptable carrier.

As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The formulations may be prepared using conventional dissolution and mixing procedures. For example, the bulk drug substance (i.e., compound of the present disclosure or stabilized form of the compound (e.g., complex with a cyclodextrin derivative or other known complexation agent)) is dissolved in a suitable solvent in the presence of one or more of the excipients described above. The compound of the present disclosure is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compounds, or pharmaceutically acceptable salts thereof described herein may be used to decrease or inhibit the activity of TYK2 or to otherwise affect the properties and/or behavior of TYK2, e.g., stability, phosphorylation, kinase activity, interactions with other proteins, etc.

### Methods of Use

In some embodiment, the present disclosure provides a method of inhibiting TYK2 activity in a subject in need thereof comprising administering to the subject an effective amount of at least one compound described herein, or a pharmaceutically acceptable salt thereof, or a pharmaceutical composition described herein.

As used herein, the term “inhibit”, “inhibition” or “inhibiting” refers to the reduction or suppression of a given condition, symptom, or disorder, or disease, or a significant decrease in the baseline activity of a biological activity or process.

One embodiment of the present disclosure is a method of treating a disease or disorder responsive to inhibition of TYK2 in a subject comprising administering to the subject an effective amount of at least one compound described herein, or a pharmaceutically acceptable salt thereof, or a pharmaceutical composition described herein. In some embodiment, the method described herein treats the disease or disorder responsive to inhibition of TYK2, wherein the disease or disorder includes inflammation, autoimmune disease, neuroinflammation, arthritis, rheumatoid arthritis, spondyloarthropathies, systemic lupus erythematous, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis and sunburn.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis.

The term “inflammatory disorders” includes diseases or disorders involving acute or chronic inflammation such as allergies, asthma, atopic dermatitis, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer).

As used herein, the term “subject” and “patient” may be used interchangeably, and means a mammal in need of treatment, e.g., human, companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The a compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)). The protection of functional groups by protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present disclosure having at least one salt-forming group may be prepared in a manner known to those skilled in the art. For example, acid addition salts of compounds of the present disclosure are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a commercially available chiral HPLC column.

The disclosure further includes any variant of the present processes, in which the reaction components are used in the form of their salts or optically pure material. Compounds of the disclosure and intermediates can also be converted into each other according to methods generally known to those skilled in the art.

For illustrative purposes, the reaction described below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### EXEMPLIFICATIONS

The compounds of the Examples were analyzed or purified according to one of the Purification Methods referred to below unless otherwise described.
Aq. means aqueous;
Bn means benzyl;
Boc means tert-butoxy carbonyl;
(BPin)₂ means bis(pinacolato)diboron;
br means broad;
t-BuBrettphos Pd G3 means [(2-di-tert-butylphosphino-3,6-dimethoxy-2′,4′,6′-triisopropyl-1,1′-biphenyl)-2-(2′-amino-1,1′-biphenyl)]palladium(II) methanesulfonate;
t-BuOH means tertiary butanol;
n-BuLi means n-butyl lithium;
° C. means degrees Celsius;
CDCl₃ means deutero-chloroform;
Cs₂CO₃ means cesium carbonate;
δ means chemical shift;
d means doublet;
DAST means (diethylamino)sulfur trifluoride;
dd means double doublet;
DCE means 1,2-dichloroethane;
DCM means dichloromethane;
DEA means diethylamine;
DIBAL-H means diisobutylaluminium hydride;
DIPEA means N-ethyldiisopropylamine or N,N-diisopropylethylamine;
DMA means N,N-dimethylacetamide;
DMAP means 4-dimethylaminopyridine;
DMEDA means N,N′-dimethylethane-1,2-diamine;
DMF means N,N-dimethylformamide;
DMSOmeans Dimethylsulfoxide;
DMSO-d₆ means hexadeuterodimethyl sulfoxide;
EDCI means N-(3-dimethylaminopropyl)-N′-ethylcarbodiimide hydrochloride;
Et means ethyl;
EtOH means ethanol;
EtOAc means ethyl acetate;
Eq. means equivalent;
g means gram;
HCl means hydrochloric acid;
HCO₂H means formic acid;
¹H NMR means proton nuclear magnetic resonance;
H₂O means water;
HOBt means 1-hydroxybenzotriazole;
HPLC means high pressure liquid chromatography;
h means hour;
IPA means 2-propanol;
K₂CO₃ means potassium carbonate;
KF means potassium fluoride;
KOH means potassium hydroxide;
K₃PO₄ means potassium phosphate tribasic;
L means litre;
LCMS means liquid chromatography mass spectrometry;
LDA means lithium diisopropylamide;
m means multiplet;
M means molar;
Me means methyl;
Me₄t-BuXPhos means methanesulfonato (2-di-tert-butylphosphino-3,4,5,6-tetramethyl-2′,4′,6′-triisopropyl-1,1-biphenyl)(2′-amino-1,1′-biphenyl-2-yl) palladium(II);
MeCN means acetonitrile;
Mel means iodomethane;
MeOH means methanol;
MeOD-d₄ means deutero-methanol;
mg means milligram;
MgSO₄ means magnesium sulfate;
MHz means mega Hertz;
mins means minutes;
mL means millilitres;
mmol means millimole;
MS m/z means mass spectrum peak;
MsCl means methanesulfonyl chloride;
N₂ means nitrogen;
NaBH₄ means sodium borohydride;
NaBH₃CN means sodium cycanoborohydride;
Na₂CO₃ means sodium carbonate;
NaH means sodium hydride;
NaHCO₃ means sodium bicarbonate;
NaIO₄ means sodium periodate;
NaOH means sodium hydroxide;
Na₂SO₄ means sodium sulfate;
NBS means N-bromosuccinimide;
NH₃ means ammonia;
NH₄Cl means ammonium chloride;
NH₄OH is ammonium hydroxide;
P(cy)₃ means tricyclohexylphospine;
Pd(amphos)Cl₂ means Bis(di-tert-butyl(4-dimethylaminophenyl)phosphine)dichloropalladium(II);
Pd(t-Bu₃P)₂ means bis(tri-tert-butylphosphine)palladium(O);
Pd₂(dba)₃ means tris(dibenzylideneacetone)dipalladium(O);
Pd(dppf)Cl₂ means [1,1′-bis(diphenylphosphino)ferrocene]dichloropalladium(II);
Pd(dtbpf)Cl₂ means [1,1′-Bis(di-tert-butylphosphino)ferrocene]dichloropalladium(II); PdCl₂(PPh₃)₂ means bis(triphenylphosphine)palladium(II) dichloride;
Pd/C means palladium on charcoal;
Pd-132 means dichlorobis[di-tert-butyl(4-dimethylaminophenyl)phosphine]palladium(II) PE means petroleum ether;
PEPPSI-IPr catalyst means [1,3-bis(2,6-Diisopropylphenyl)imidazol-2-ylidene](3-chloropyridyl)palladium(II) dichloride;
PMB means para-methoxybenzyl;
PMBCl means para-methoxybenzyl choride;
POCl₃ means phosphorous oxychloride;
q means quartet;
rt means room temperature;
RT means retention time;
RuCl₃ means ruthenium (III) chloride;
RuPhos Pd G3 means (2-dicyclohexylphosphino-2′,6′-diisopropoxy-1,1′-biphenyl)[2-(2′-amino-1,1′-biphenyl)]palladium(II) methanesulfonate;
s means singlet;
sat. means saturated;
SEM means [2-(trimethylsilyl)ethoxy]methyl acetal;
Select-F means 1-chloromethyl-4-fluoro-1,4-diazoniabicyclo[2.2.2]octane
bis(tetrafluoroborate);
SFC means supercritical fluid chromatography;
SOCl₂ means thionyl chloride;
soln. means solution;
t means triplet;
TBAB means tetra-n-butylammonium bromide;
TBAF means tetrabutylammonium fluoride;
TBME means tert-butyl methyl ether;
TEA means triethylamine;
TFA means trifluoroacetic acid;
Tf₂O means triflic anhydride;
THF means tetrahydrofuran;
THP means tetrahydropyran;
TLC means thin layer chromatography;
TMSCN means trimethylsilyl cyanide;
TsCl means para-toluenesulfonyl chloride;
TsOH means para-toluenesulfonic acid
μL means micro litres;
μmol means micromole;
Xantphos means 4,5-Bis(diphenylphosphino)-9,9-dimethylxanthene;
XPhos Pd G3 means (2-dicyclohexylphosphino-2′,4′,6′-triisopropyl-1,1′-biphenyl)[2-(2′-amino-1,1′-biphenyl)]palladium(II) methanesulfonate RuPhos Pd G3 means (2-dicyclohexylphosphino-2′,6′-diisopropoxy-1,1′-biphenyl)[2-(2′-amino-1,1′-biphenyl)]palladium(II) methanesulfonate According to a first process, compounds of Formula (I-1), may be prepared from compounds of Formulae (II) and (III), as shown in Scheme 1.

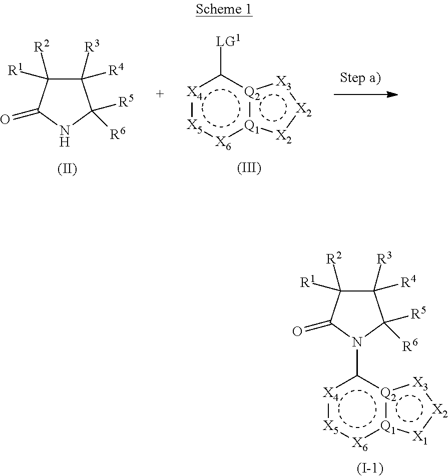

LG¹ is a leaving group, typically a halide, mesylate or triflate and preferably Cl or triflate.

The compound of Formula (I-1) may be prepared according to process step (a) a Buchwald-Hartwig cross coupling reaction. Typical conditions comprise, reaction of the amide of Formula (II) with the compound of Formula (III) in the presence of a suitable inorganic base, a suitable palladium catalyst in the presence of suitable phosphine ligands, in a suitable solvent at elevated temperature, optionally under microwave irradiation. Preferred conditions comprise, reaction of the compounds of Formulae (II) and (III) in the presence of RuPhos PdG3, Xantphos or Me₄t-BuXPhos, optionally in combination with Pd₂(dba)₃, in the presence of a suitable base such as K₂CO₃, K₃PO₄ or Cs₂CO₃ in a suitable solvent such as dioxane, THE or toluene at between 25° C. and 120° C., optionally under microwave irradiation.

According to a second process, compounds of Formula (I-1A), wherein X₂ is C—R^{S}, may be prepared from compounds of Formulae (II), (IV) and (V), as shown in Scheme 2.

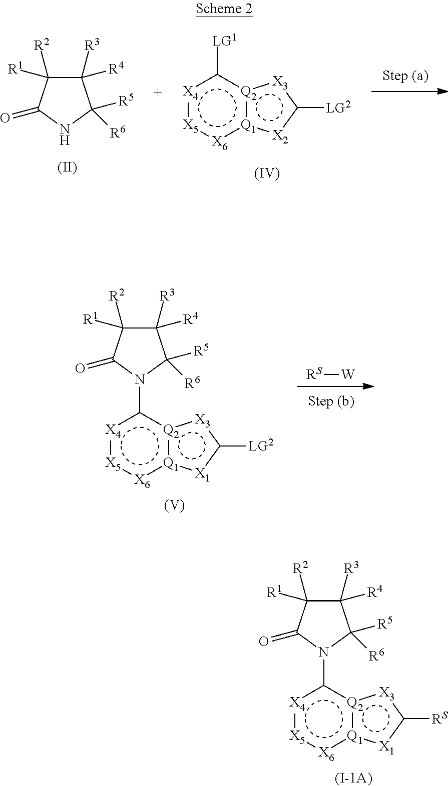

LG¹ and LG² are leaving groups, typically a halide, mesylate or triflate and preferably Cl, Br or triflate. W is a boronic acid or suitable boronate ester, such as boronic acid pinacol ester.

The compound of Formula (V) may be obtained from the compounds of Formulae (II) and (IV), according to process step a), as previously described in Scheme 1.

Step b) The compound of Formula (I-1A) may be prepared from the compound of Formula (V) and R^{S}W, by a palladium catalysed cross-coupling reaction. Typical cross-coupling reaction conditions comprise a palladium catalyst containing suitable phosphine ligands, in the presence of an inorganic base, in a suitable solvent at between rt and the reflux temperature of the reaction, optionally in the presence of microwave irradiation. Preferred conditions comprise, reaction of the compound of Formula (V) and R^{S}W, in the presence of XPhos Pd G3, Pd(dppf)Cl₂, Pd(amphos)Cl₂ or Pd(dtbpf)Cl₂, and a suitable base such as Na₂CO₃, K₂CO₃, K₃PO₄ or KF in a suitable solvent such as dioxane or toluene, optionally in the presence of water, at between 70° C. and 100° C.

According to a third process, wherein, Q₁ and Q₂ are C, X₁ and X₃ are N and X₂ is C—R^{S}, compounds of Formula (I-1B), may be prepared from compounds of Formulae (II), (VI), (VII) and (VIII), as shown in Scheme 3.

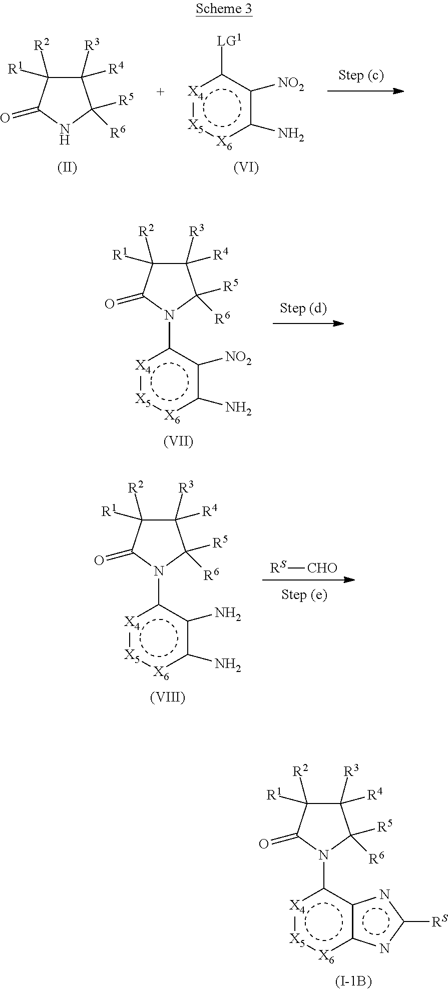

Step c) The compound of Formula (VII) may be prepared by reaction of the compounds of Formulae (II) and (VI), in the presence of a suitable strong base and a suitable aprotic polar solvent at between 0° C. and elevated temperature. Preferred conditions, comprise reaction of the compound of Formula (II) with the compound of Formula (VI) in the presence of NaH in DMSO or DMF at ambient temperature.

Step d) The compound of Formula (VIII) may be prepared from the compound of Formula (VII) by a reduction reaction, typically in the presence of a suitable hydrogenation catalyst, such as Pd/C in a suitable alcoholic solvent such as MeOH under an atmosphere of H₂.

Step e) The compound of Formula (I-1B) may be prepared from the di-amine of Formula (VIII) and the aldehyde, RSCHO, via a condensation reaction in the presence of a suitable acidic catalyst, such as TsOH in a suitable polar aprotic solvent, such as DMF at elevated temperature, such as between 50° C. and 80° C.

According to a fourth process, compounds of Formula (I-1C), wherein R^{X5} is not H, may be prepared from the compounds of Formulae (II), (IX) and (X), as shown in scheme 4.

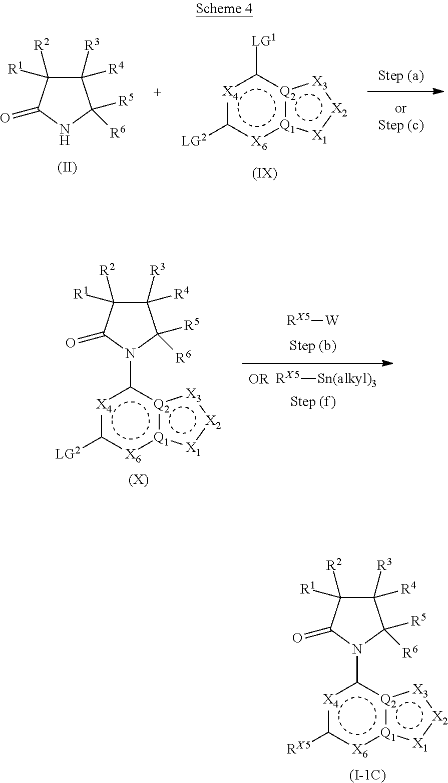

W is a boronic acid or suitable boronate ester, such as boronic acid pinacol ester.

The compound of Formula (X) may be prepared from the compounds of Formulae (II) and (IX) according to process step (a) as previously described in scheme 1, or according to process step c) as previously described in Scheme 3.

The compound of Formula (I-1C) may be prepared from the compound of Formula (X) and R^{X5}W by a Suzuki type cross-coupling reaction, as previously described in step b) in Scheme 2. Preferred conditions comprise reaction of the compound of Formula (X) with R^{X5}W in the presence of PEPPSI-IPR catalyst, BrettPhos Pd G3, t-BuBrettphos Pd G3, Pd(t-Bu₃P)₂, Pd(dtbpf)Cl₂, or Pd(amphos)Cl₂ and a suitable base such as CsF, K₂CO₃ or K₃PO₄ in a suitable solvent such as aqueous dioxane or t-amyl alcohol at between 70° C. and 100° C.

Alternatively, the compound of Formula (I-1C) may be prepared from the compound of Formula (X) and R^{X5}Sn(alkyl)₃ according to process step (f), a palladium catalysed cross-coupling reaction with a suitable alkyl or aryl stannane, a Stille Reaction. Typical cross-coupling reaction conditions comprise a palladium catalyst containing suitable phosphine ligands, in the presence of an inorganic base, in a suitable aqueous solvent at between rt and the reflux temperature of the reaction. Preferred conditions comprise reaction of the compound of Formula (X) with R^{X5}Sn(butyl)₃, in the presence of a suitable catalyst such as Pd(OAc)₂, with a suitable ligand such as P(Cy)₃ optionally in the presence of an additive, typically CsF, in a suitable solvent such as dioxane, at elevated temperature, such as 90-100° C.

According to a fifth process, compounds of Formula (I-1A) may be prepared from the compounds of Formulae (V) and (XI), as shown in scheme 5.

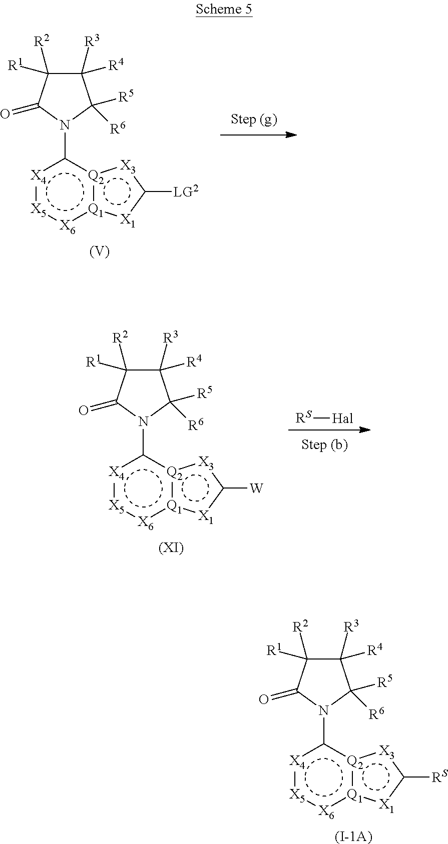

Hal is halogen and preferably Cl, Br or I.

Step f) The compound of Formula (XI) may be prepared from the compound of Formula (V), according to step (g), a boronate ester formation achieved by treatment with a suitable boronate such as (BPin)₂, in the presence of a suitable inorganic base, such as K₂CO₃ or KOAc and a suitable catalyst, such as, Pd(dppf)Cl₂ or Pd₂(dba)₃ in a suitable non-polar solvent, such as MeCN at between rt and elevated temperature.

The compound of Formula (I-1A) may be prepared from the compound of Formula (XI) according to process step b), a Suzuki coupling reaction, as previously described in Schemes 2 and 4.

According to a sixth process, compounds of Formula (I-1A) may be prepared from the compounds of Formulae (II), (IV) and (IIIA) as shown in scheme 6

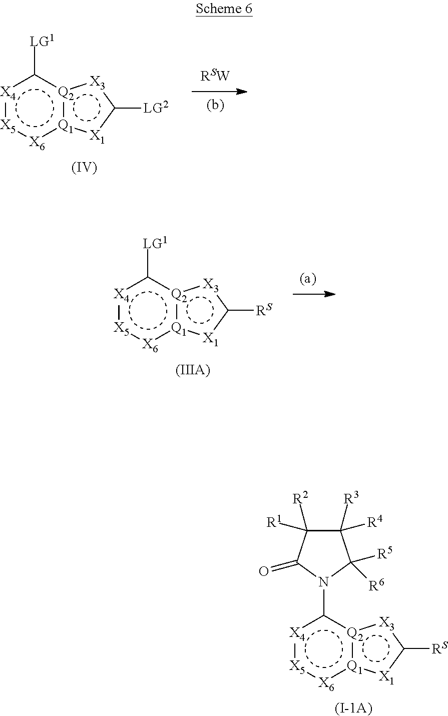

The compound of Formula (IIIA) may be prepared from the compound of Formula (IV) and R^{S}W according to process step b), a Suzuki coupling reaction, as previously described in Schemes 2 and 4. Preferred conditions comprise reaction of the compound of Formula (IV) with R^{S}W, in the presence of Pd(OAc)₂, PPh₃, Na₂CO₃, in aqueous toluene at about 110° C.

The compound of Formula (I-1A) may be obtained from the compounds of Formulae (IIIA) and (II) according to process step (a) as previously described in Scheme 1.

According to a seventh process, compounds of Formula (I-1A), may be prepared from the compound of Formula (V) as shown in Scheme 7.

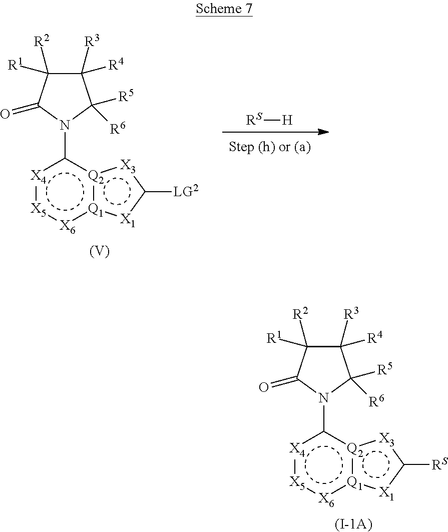

Wherein R^{S} is N-linked, the compounds of Formula (I-1A) may be prepared according to step (h), a metal catalysed cross coupling reaction such as an Ullmann-type, copper mediated coupling reaction. Typical conditions comprise, reaction of the compound of Formula (V) with the RSH, a copper catalyst, a suitable ligand, in the presence of a suitable inorganic or organic base in a suitable solvent at elevated temperature. Preferred conditions comprise, reaction of the compound of Formula (V) with RSH in the presence of CuI, a suitable ligand such as N1,N2-dimethylethane-1,2-diamine, L-proline, 4,7-dimethoxy-1,10-phenanthroline or pyridine-2-carboxylic acid, a suitable inorganic base such as K₂CO₃, Cs₂CO₃, K₃PO₄ or KOtBu, optionally in a solvent such as dioxane, toluene or DMSO at between 90° C. and 120° C. and optionally under microwave irradiation.

Alternatively, the compound of Formula (I-1A) may be prepared from the compound of Formula (V) and RSH according to process step (a) a Buchwald type cross coupling, as previously described in Scheme 1.

According to an eighth process, compounds of Formula (I-1C), wherein R^{X5} is not H, may be prepared from the compounds of Formulae (X) and (XII), as shown in scheme 8

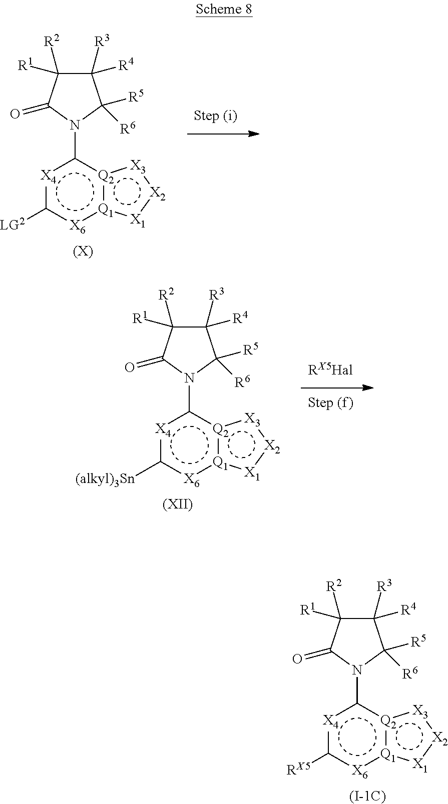

The compound of Formula (XII) may be prepared from the compound of Formula (X) according to process step (i), the formation of an alkyl tin compound. Preferred conditions comprise reaction of the compound of Formula (X) with Sn(Bu)₃, in the presence of Pd(dtbpf)Cl₂, in toluene at about 100° C.

The compound of Formula (I-IC) may be prepared from the compound of Formula (XII) and R^{X5}Hal according to process step (f) as previously described in Scheme 4.

According to a ninth process, compounds of Formula (I-2) may be prepared from the compounds of Formulae (II) and (XIII), as shown in scheme 9.

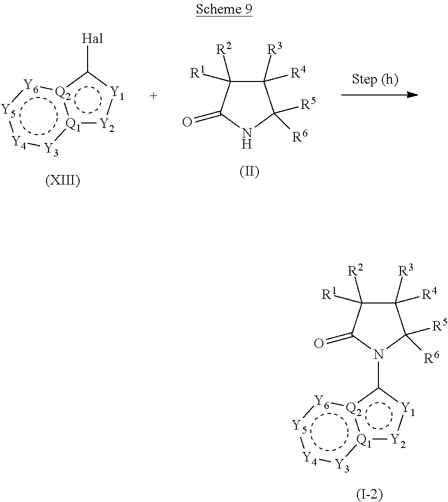

The compound of Formula (I-2) may be prepared from the compounds of Formulae (XIII) and (II) according to step (h), as previously described in Scheme 7.

Compounds of Formulae (I-1), (I-2), (I-1A), (I-1B), (I-1C), (V) or (X) may be converted to alternative compounds of Formulae (I-1). (I-2), (I-1A), (I-1B), (I-1C), (V) or (X), by standard chemical transformations such as for example, the transformation steps described above, alkylation of a heteroatom such as N, via reductive amination, alkylation in the presence of an inorganic base, palladium or copper mediated Suzuki type reactions, copper carbenoid mediated N-alklyations, or halogenation, such as fluorination, using methods well known to those skilled in the art.

The compounds of Formulae (II), (III), (IV), (VI), (IX) and (XIII) are commercially available, may be prepared by analogy to methods known in the literature, or the methods described in the Experimental section below.

It will be appreciated by those skilled in the art that it may be necessary to utilise a suitable protecting group strategy for the preparation of compounds of Formulae (I-1) and (I-2). Typical protecting groups may comprise, carbamate and preferably Boc for the protection of amines, a Tosyl group for the protection of imidazole N atoms, a THP or SEM group for the protection of pyrazole N atoms and a PMB group for the protection of an amide N.

### HPLC Codes

| HPLC Code | Conditions |
|---|---|
| HPLC-1 | Phenomenex Synergi C18 150 × 30 mm, 4 μm, MeCN/0.225% HCO₂H, |
| | gradient 0-100% optimised for each example |
| HPLC-2 | Boston Prime C18 150 × 30 mm, 5 μm; MeCN/H₂O (0.05% NH₄OH + |
| | 10 mM NH₄HCO₃), gradient 0-100% optimised for each example |
| HPLC-3 | Welch Xtimate C18 150 × 25 mm, 5 μm; MeCN/H₂O (10 mM NH₄HCO₃), |
| | gradient 0-100% optimised for each example |
| HPLC-4 | Agela DuraShell C18 150 × 25 mm, 5 μm; MeCN/H2O (0.05% NH₄OH + |
| | 10 mM NH₄HCO₃), gradient 0-100% optimised for each example |
| HPLC-5 | Phenomenex Luna C8, 250 × 50 mm, 10 μm; 40-70% MeCN/H₂O |
| | (NH₄OH + NH₄HCO₃), gradient 0-100% optimised for each sample |
| HPLC-6 | Boston Uni C18, 150 × 40 mm, 5 μm; MeCN/H₂O (0.1% TFA); gradient |
| | 0-100% optimised for each example |
| HPLC-7 | Boston Prime C18 150 × 30 mm, 5 μm; MeCN/H₂O (0.05% NH₄OH), |
| | gradient 0-100% optimised for each example |
| HPLC-8 | Boston Prime ODS 150 × 30 mm, 5 μm; MeCN/H₂O (0.05% HCl), gradient |
| | 0-100% optimised for each example |
| HPLC-9 | Waters SunFire C18 OBD 100 × 30 mm, 5 μm; MeCN/H₂O (+0.1% TFA), |
| | gradient 0-100% optimised for each sample |
| HPLC-10 | Waters XSelect C18 OBD 30 × 100 mm, 5 μm; MeCN/H₂O (+0.2% |
| | NH₄OH), gradient 0-100% optimised for each sample |
| HPLC-11 | Waters Xbridge C18 100 × 25 mm, 5 μm; MeCN/H₂O (0.225% HCO2H), |
| | gradient 0-100% optimised for each sample |
| HPLC-12 | Waters Xbridge C18 100 × 25 mm, 5 μm; MeCN/H₂O (0.1% TFA), |
| | gradient 0-100% optimised for each sample |
| HPLC-13 | Waters Xbridge C18 100 × 25 mm, 5 μm; MeCN/H₂O (0.225% TFA), |
| | gradient 0-100% optimised for each sample |
| HPLC-14 | Welch Xtimate C18 150 × 25 mm, 5 μm; MeCN/H₂O (0.1% TFA), gradient |
| | 0-100% optimised for each example |
| HPLC-15 | Welch Xtimate C18, 150 × 25 mm, 5 μm; MeCN/H₂O (0.075% TFA), |
| | gradient 0-100% optimised for each example |
| HPLC-16 | Welch Xtimate C18, 150 × 25 mm, 5 μm; MeCN/H₂O (0.225% HCO₂H), |
| | gradient 0-100% optimised for each example |
| HPLC-17 | YMC-Actus Triart C18 100 × 30 mm; 5 μm, MeCN/H₂O (0.1% TFA), |
| | gradient 0-100% optimised for each sample |
| HPLC-18 | Boston Prime C18, 150 × 30 mm, 5 μm; MeCN/H₂O (0.225% HCO₂H), |
| | gradient 0-100% optimised for each sample |
| HPLC-19 | Boston Prime C18, 150 × 30 mm, 5 μm; MeCN/H₂O (10 mM NH₄HCO₃), |
| | gradient 0-100% optimised for each sample |
| HPLC-20 | Boston Green ODS, 150 × 30 mm, 5 μm; MeCN/H₂O (0.225% HCO₂H); |
| | gradient 0-100% optimised for each example |
| HPLC-21 | Boston Green ODS 150 × 30 mm, 5 μm; MeCN/H₂O (10 mM NH₄HCO₃), |
| | gradient 0-100% optimised for each example |
| HPLC-22 | YMC-Actus Triart C18 150 × 40 mm; 7 μm, MeCN/H₂O |
| | (NH₃H₂O + NH₄HCO₃), gradient 0-100% optimised for each sample |
| HPLC-23 | YMC-Park CN C18 150 × 30 mm; 5 μm, MeCN/H₂O (0.225% HCO₂H), |
| | gradient 0-100% optimised for each sample |

### Preparation 1

### 3-ethyl-2-oxopyrrolidine-3-carbonitrile

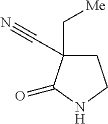

Part A: To a solution of ethyl 2-cyanobutanoate (20 g, 141.7 mmol) in THE (450 mL) was added sodium tert-butoxide (15 g, 156 mmol) and then a solution of tert-butyl 1,2,3-oxathiazolidine-3-carboxylate 2,2-dioxide (33 g, 147.8 mmol) in THE (350 mL) was carefully added dropwise, and the reaction stirred overnight at rt. The reaction mixture was neutralized with 0.1 M HCl and extracted with EtOAc. The combined organics were washed successively with water and saturated brine, dried (MgSO₄) and evaporated under reduced pressure to give ethyl 4-((tert-butoxycarbonyl)amino)-2-cyano-2-ethylbutanoate (40 g) which was used without further purification.

Part B: Ethyl 4-((tert-butoxycarbonyl)amino)-2-cyano-2-ethylbutanoate (Part A) was dissolved in dioxane and 10% HCl dioxane solution (3 eq.) added. The reaction mixture was stirred overnight at rt, then evaporated under reduced pressure to give ethyl 4-amino-2-cyano-2-ethylbutanoate hydrochloride, which was used without further purification.

Part C: Ethyl 4-amino-2-cyano-2-ethylbutanoate hydrochloride (Part B) was dissolved in EtOH (25V), NaHCO₃ (2 eq.) was added and the reaction mixture was stirred for 48 h at 40° C. After cooling, the reaction mixture was filtered and the filtrate evaporated under reduced pressure. The residue was purified by column chromatography (SiO₂) to give 3-ethyl-2-oxopyrrolidine-3-carbonitrile (3 g, 16.5%). ¹H NMR (400 MHz, CDCl₃) δ: 6.90 (s, 1H), 3.54 (ddd, 1H), 3.47-3.39 (m, 1H), 2.62 (ddd, 1H), 2.23 (ddd, 1H), 2.16-2.02 (m, 1H), 1.76 (dq, 1H), 1.18 (td, 3H).

### Preparation 2

### 3-cyclobutyl-2-oxopyrrolidine-3-carbonitrile

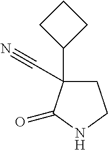 [SMILES: N#CC1(C2CCC2)CCNC1=O]

3-Cyclobutyl-2-oxopyrrolidine-3-carbonitrile was prepared from tert-butyl 1,2,3-oxathiazolidine-3-carboxylate 2,2-dioxide and ethyl 2-cyano-2-cyclobutylacetate, following the procedure described in Preparation 1. ¹H NMR (400 MHz, CDCl₃) δ 6.82 (s, 1H), 3.49 (td, 1H), 3.44-3.32 (m, 1H), 2.76 (p, 1H), 2.56 (dt, 1H), 2.20-2.16 (m, 5H), 1.94 (p, 2H).

### Preparation 3

### ethyl 2-cyano-2-(oxetan-3-yl)acetate

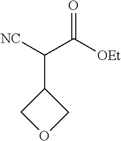 [SMILES: CCOC(=O)C(C#N)C1COC1]

To a solution of oxetan-3-one (50 g, 0.69 mol) and ethyl 2-cyanoacetate (78.5 g, 0.69 mol) in DMSO (1 L) was added diethyl 1,4-dihydro-2,6-dimethyl-3,5-pyridinedicarboxylate (175.7 g, 0.69 mol) followed by L-proline (159.8 g, 1.39 mol) and the mixture stirred at 25° C. for 48 h. The reaction mixture was poured into 2 L saturated citric acid solution and extracted with EtOAc (4×500 mL). The combined organics were washed with saturated aq. Na₂CO₃ (300 mL), dried (MgSO₄) and evaporated to dryness in vacuo. The residue was purified by reduced-pressure distillation (internal temperature: 100° C.) and followed by column chromatography (SiO₂, 25%-50% EtOAc/PE) to give ethyl 2-cyano-2-(oxetan-3-yl)acetate as a light yellow oil (81 g, 69%). ¹HNMR (400 MHz, CDCl₃) δ: 4.90 (dd, 2H), 4.61 (dt, 2H), 4.29 (q, 2H), 3.91 (d, 1H), 3.67-3.43 (m, 1H), 1.35 (t, 3H)

### Preparation 4

### ethyl 2-cyano-2-cyclopropylacetate

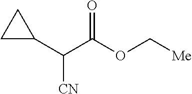 [SMILES: CCOC(=O)C(C#N)C1CC1]

To a solution of 2-cyclopropylacetonitrile (200 g, 2.47 mol) in DMF (2.2 L) was added CO(OEt)₂ (728 g, 6.16 mol) followed by t-BuOK (719 g, 6.41 mol) in small portions at 20° C. After the addition was complete the mixture was heated at 50° C. for 20 hrs. The reaction mixture was cooled to rt and poured into 4.0 L ice-water. The mixture was extracted with MTBE (3×1 L). The combined organics were dried (MgSO₄) and evaporated to dryness in vacuo. The residue was purified by reduced-pressure distillation (External temperature: 100° C.; Internal temperature: 59° C.) to afford ethyl 2-cyano-2-cyclopropylacetate as a yellow oil (300 g, 79%). ¹H NMR (400 MHz, CDCl₃) δ: 4.31 (q, 2H), 3.25 (d, 1H), 1.51-1.22 (m, 4H), 0.82-0.67 (m, 2H), 0.63-0.40 (m, 2H).

### Preparation 5

### ethyl (3R)-4-((tert-butoxycarbonyl)amino)-2-cyano-2-cyclopropyl-3-methylbutanoate

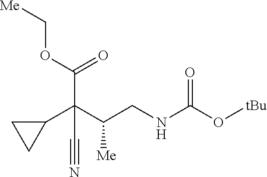 [SMILES: CCOC(=O)C(C#N)(C1CC1)[C@@H](C)CNC(=O)OC(C)(C)C]

Cs₂CO₃ (103 g, 316 mmol) and TBAB (13.6 g, 42.2 mmol) were added to a solution of tert-butyl (R)-5-methyl-1,2,3-oxathiazolidine-3-carboxylate 2,2-dioxide (50 g, 211 mmol) and ethyl 2-cyano-2-cyclopropylacetate (Preparation 4, 32.3 g, 211 mmol) in toluene (500 mL) and the reaction stirred at 30° C. for 8 h. The pH of the mixture was adjusted to ~5 by progressively adding 1N HCl at 0-10° C. and the mixture stirred for 30 min and extracted with EtOAc (2×100 mL). The combined organics were dried (MgSO₄) and evaporated under reduced pressure to afford ethyl (3R)-4-((tert-butoxycarbonyl)amino)-2-cyano-2-cyclopropyl-3-methylbutanoate as a colourless oil (65 g, 99%).

### Preparation 6

### ethyl (3S)-4-((tert-butoxycarbonyl)amino)-2-cyano-2-cyclopropyl-3-methylbutanoate

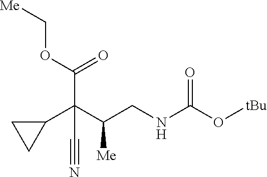 [SMILES: CCOC(=O)C(C#N)(C1CC1)[C@H](C)CNC(=O)OC(C)(C)C]

Ethyl (3S)-4-((tert-butoxycarbonyl)amino)-2-cyano-2-cyclopropyl-3-methylbutanoate was prepared as a colourless oil (60 g, 92%) from tert-butyl (S)-5-methyl-1,2,3-oxathiazolidine-3-carboxylate 2,2-dioxide and ethyl 2-cyano-2-cyclopropylacetate (Preparation 4) following the procedure described in Preparation 5.

### Preparation 7

### ethyl (3S)-4-((tert-butoxycarbonyl)amino)-2-cyano-3-methyl-2-(oxetan-3-yl)butanoate

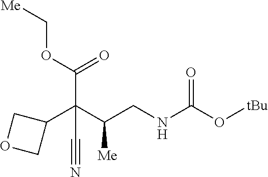 [SMILES: CCOC(=O)C(C#N)(C1COC1)[C@H](C)CNC(=O)OC(C)(C)C]

Ethyl (3S)-4-((tert-butoxycarbonyl)amino)-2-cyano-3-methyl-2-(oxetan-3-yl)butanoate was obtained as a yellow oil (102 g, 93%) from tert-butyl (S)-5-methyl-1,2,3-oxathiazolidine-3-carboxylate 2,2-dioxide and ethyl 2-cyano-2-(oxetan-3-yl)acetate (Preparation 3), following a similar procedure to that described in Preparation 5.

### Preparation 8

### ethyl 4-((tert-butoxycarbonyl)amino)-2-cyano-2-(oxetan-3-yl)butanoate

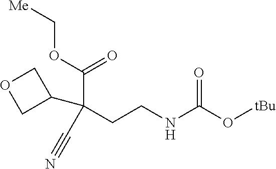 [SMILES: CCOC(=O)C(C#N)(CCNC(=O)OC(C)(C)C)C1COC1]

Ethyl 4-((tert-butoxycarbonyl)amino)-2-cyano-2-(oxetan-3-yl)butanoate was prepared as a yellow oil (17.2 g, 93.2%) from ethyl 2-cyano-2-(oxetan-3-yl)acetate (Preparation 3) and tert-butyl 1,2,3-oxathiazolidine-3-carboxylate 2,2-dioxide, following the procedure described in Preparation 5.

### Preparation 9

### (4R)-3-cyclopropyl-4-methyl-2-oxopyrrolidine-3-carbonitrile

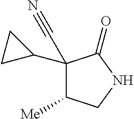 [SMILES: C[C@H]1CNC(=O)C1(C#N)C1CC1]

TsOH (79.7 g, 418 mmol) was added to a solution of ethyl (3R)-4-((tert-butoxycarbonyl)amino)-2-cyano-2-cyclopropyl-3-methylbutanoate (Preparation 5, 65.0 g, 209 mmol) in EtOAc (500 mL) and the reaction stirred at 50° C. for 1 h. The mixture was concentrated in vacuo, the residue dissolved in MeCN (500 mL) and K₂CO₃ (86.8 g, 628 mmol) was added and the mixture stirred at 50° C. for 5 h. The cooled reaction mixture was filtered, the filtrate was concentrated in vacuo and the crude product was purified by column chromatography (SiO₂, 50-100% EtOAc/PE) to afford (4R)-3-cyclopropyl-4-methyl-2-oxopyrrolidine-3-carbonitrile as a colourless oil (22 g, 64%). ¹HNMR: (400 MHz, CDCl₃) δ: 7.49 (d, 1H), 3.53 (dd, 1H), 3.16-3.02 (m, 1H), 2.56 (dt, 1H), 1.32 (dd, 3H), 1.14-0.91 (m, 1H), 0.89-0.48 (m, 4H).

### Preparation 10

### (4S)-3-cyclopropyl-4-methyl-2-oxopyrrolidine-3-carbonitrile

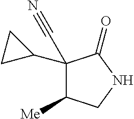 [SMILES: C[C@@H]1CNC(=O)C1(C#N)C1CC1]

(4S)-3-Cyclopropyl-4-methyl-2-oxopyrrolidine-3-carbonitrile was prepared (20 g, 63%) from ethyl (3S)-4-((tert-butoxycarbonyl)amino)-2-cyano-2-cyclopropyl-3-methylbutanoate (Preparation 6), following the procedure described in Preparation 9. ¹HNMR (400 MHz, CDCl₃) δ: 7.49 (d, 1H), 3.53 (dd, 1H), 3.16-3.02 (m, 1H), 2.56 (dt, 1H), 1.32 (dd, 3H), 1.14-0.91 (m, 1H), 0.89-0.48 (m, 4H)

### Preparation 11

### (4S)-4-methyl-3-(oxetan-3-yl)-2-oxopyrrolidine-3-carbonitrile

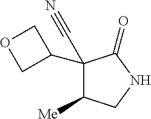 [SMILES: C[C@@H]1CNC(=O)C1(C#N)C1COC1]

A solution of ethyl (3S)-4-((tert-butoxycarbonyl)amino)-2-cyano-3-methyl-2-(oxetan-3-yl)butanoate (Preparation 7, 100 g, 306 mmol) in THF (1 L) was cooled to 0° C. NaH (18.38 g, 459 mmol) was added portion wise and the mixture allowed to warm up to 20° C. and stirred for 10 h. The mixture was quenched with water (400 mL) at 0° C. and extracted with EtOAc (3×400 mL). The combined organics were dried (MgSO₄) and concentrated. The residue was purified by column chromatography (SiO₂, 50-100% EtOAc/PE) to give (4S)-4-methyl-3-(oxetan-3-yl)-2-oxopyrrolidine-3-carbonitrile as a yellow oil (34 g, 61.6%). ¹HNMR (400 MHz, CDCl₃) δ: 7.15 (d, 1H), 5.03 (dt, 1H), 4.95-4.63 (m, 3H), 3.58 (dd, 1H), 3.48 (ddd, 2H), 3.14 (dd, 1H), 3.00 (dt, 1H), 2.40 (dd, 1H), 1.32 (d, 2H), 1.10-0.84 (m, 1H)

### Preparation 12

### 3-(oxetan-3-yl)-2-oxopyrrolidine-3-carbonitrile

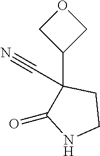 [SMILES: N#CC1(C2COC2)CCNC1=O]

3-(Oxetan-3-yl)-2-oxopyrrolidine-3-carbonitrile was obtained as a white solid, 4.1 g, 46.7%, from ethyl 4-((tert-butoxycarbonyl)amino)-2-cyano-2-(oxetan-3-yl)butanoate (Preparation 8), following the procedure described in Preparation 11. LCMS m/z=167 [M+H]⁺.

### Preparation 13

### (5R)-3-cyclopropyl-5-methyl-2-oxopyrrolidine-3-carbonitrile

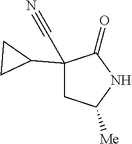 [SMILES: C[C@@H]1CC(C#N)(C2CC2)C(=O)N1]

Part A: Cs₂CO₃ (128 g, 391 mmol) and TBAB (16.8 g, 52.2 mmol) were added to ethyl 2-cyano-2-cyclopropylacetate (Preparation 4, 40 g, 261 mmol) in MeCN (500 mL) and the solution stirred at rt for 30 mins. tert-Butyl (R)-4-methyl-1,2,3-oxathiazolidine-3-carboxylate 2,2-dioxide (62 g, 261 mmol) was added and the reaction stirred at 30° C. for 10 h. The mixture was concentrated in vacuo, the residue diluted with H₂O (200 mL) and EtOAc (500 mL), and the pH adjusted to ~5 by progressively adding 1N HCl at 0-10° C. The mixture was extracted with EtOAc (500 mL and 2×200 mL) and the combined organics were washed with saturated aq. NaHCO₃ (200 mL), dried (MgSO₄) and concentrated in vacuo to give ethyl (4R)-4-((tert-butoxycarbonyl)amino)-2-cyano-2-cyclopropylpentanoate as a yellow oil (81 g).

Part B: TsOH·H₂O (98.1 g, 515 mmol) was added to a solution of ethyl (4R)-4-((tert-butoxycarbonyl)amino)-2-cyano-2-cyclopropylpentanoate (80 g, 257 mmol) in EtOAc (500 mL) and the solution stirred at 50° C. for 1 h. The mixture was concentrated in vacuo and the residue dissolved in MeCN (800 mL). K₂CO₃ (106.9 g, 773 mmol) was added and the reaction was stirred at 50° C. for 6 h. The cooled reaction mixture was concentrated in vacuo washed with H₂O (500 mL) and extracted with EtOAc (500 mL and 300 mL). The combined organics were dried (MgSO₄) and evaporated to dryness in vacuo. The residue was purified by column chromatography (SiO₂, 50-100% EtOAc/PE) to afford (5R)-3-cyclopropyl-5-methyl-2-oxopyrrolidine-3-carbonitrile as a white solid (27.1 g, 64%). ¹HNMR (400 MHz, CDCl₃) δ: 6.94 (d, 1H), 4.07-3.64 (m, 1H), 2.71 (dd, 1H), 2.50 (dd, 1H), 2.22 (dd, 1H), 1.78 (dd, 1H), 1.34 (dd, 3H), 1.28-1.12 (m, 1H), 0.87-0.50 (m, 4H).

### Preparation 14 and 15

### (3S,5R)-3-cyclopropyl-5-methyl-2-oxopyrrolidine-3-carbonitrile and (3R,5R)-3-cyclopropyl-5-methyl-2-oxopyrrolidine-3-carbonitrile

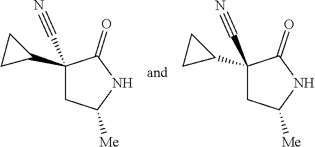 [SMILES: C[C@@H]1C[C@@](C#N)(C2CC2)C(=O)N1 ; C[C@@H]1C[C@](C#N)(C2CC2)C(=O)N1]

(5R)-3-Cyclopropyl-5-methyl-2-oxopyrrolidine-3-carbonitrile (Preparation 13, 15.0 g, 91.4 mmol) was separated by SFC (Chiralpak IC, 250×30 mm, 5 Qim; 30% MeOH (+0.1% NH₄OH) in CO₂) to afford the title compounds.

Peak 1, Preparation 14; (3S,5R)-3-cyclopropyl-5-methyl-2-oxopyrrolidine-3-carbonitrile as light yellow solid (6.80 g, 45%). LCMS m/z=165 [M+H]⁺; ¹HNMR (400 MHz, CDCl₃) δ: 6.85 (s, 1H), 3.86 (dd, 1H), 2.51 (dd, 1H), 2.23 (dd, 1H), 1.35 (d, 3H), 1.29-1.17 (m, 1H), 0.83-0.58 (m, 4H). [α]=24.2° c.=1.0 g/100 mL MeOH Peak 2, Preparation 15; (3R,5R)-3-cyclopropyl-5-methyl-2-oxopyrrolidine-3-carbonitrile as light yellow solid (7.70 g, 51%). LCMS m/z=165 [M+H]⁺; ¹T HNMR (400 MHz, CDCl₃) δ: 6.27 (s, 1H), 3.94 (dt, 1H), 2.72 (dd, 1H), 1.79 (dd, 1H), 1.31 (t, 3H), 1.23 (ddd, 1H), 0.87-0.56 (m, 4H). [α]=−42.6° c.=1.1 g/100 mL MeOH

### Preparation 16

### (5S)-3-cyclopropyl-5-methyl-2-oxopyrrolidine-3-carbonitrile

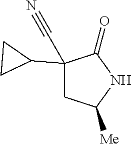 [SMILES: C[C@H]1CC(C#N)(C2CC2)C(=O)N1]

The title compound was prepared as a white solid (33 g, 77%) from ethyl 2-cyano-2-cyclopropylacetate (Preparation 4) and tert-butyl (S)-4-methyl-1,2,3-oxathiazolidine-3-carboxylate 2,2-dioxide using an analogous 2-part process as described for Preparation 13. ¹HNMR (400 MHz, CDCl₃) δ: 6.75 (d, 1H), 4.11-3.71 (m, 1H), 2.71 (dd, 1H), 2.51 (dd, 1H), 2.23 (dd, 1H), 1.79 (dd, 1H), 1.41-1.29 (m, 3H), 1.28-1.13 (m, 1H), 0.93-0.45 (m, 4H).

### Preparation 17 and 18

### (3R,5S)-3-cyclopropyl-5-methyl-2-oxopyrrolidine-3-carbonitrile and (3S,5S)-3-cyclopropyl-5-methyl-2-oxopyrrolidine-3-carbonitrile

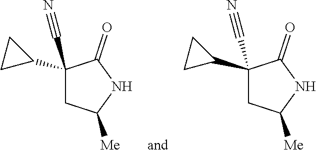 [SMILES: C[C@H]1C[C@](C#N)(C2CC2)C(=O)N1 ; C[C@H]1C[C@@](C#N)(C2CC2)C(=O)N1]

(5S)-3-Cyclopropyl-5-methyl-2-oxopyrrolidine-3-carbonitrile (Preparation 16, 15.0 g, 91.4 mmol) was separated by SFC (Chiralpak IC, 250×30 mm, 5 □m; 30% MeOH (+0.1% NH₄OH) in CO₂) to afford the title compounds.

Peak 1, Preparation 17; (3R,5S)-3-cyclopropyl-5-methyl-2-oxopyrrolidine-3-carbonitrile as light yellow solid (6.60 g). LCMS m/z=165 [M+H]⁺; ¹HNMR (400 MHz, CDCl₃) δ: 6.60 (s, 1H), 3.94 (dt, 1H), 2.72 (dd, 1H), 1.79 (dd, 1H), 1.34 (t, 3H), 1.23 (tt, 1H), 0.98-0.40 (m, 4H). [α]=−25.2° c.=1.0 g/100 mL MeOH

Peak 2, Preparation 18; (3S,5S)-3-cyclopropyl-5-methyl-2-oxopyrrolidine-3-carbonitrile as light yellow solid (7.7 g). LCMS m/z=165 [M+H]⁺; ¹HNMR (400 MHz, CDCl₃) δ: 6.27 (s, 1H), 3.94 (dt, 1H), 2.72 (dd, 1H), 1.79 (dd, 1H), 1.31 (t, 3H), 1.23 (ddd, 1H), 0.87-0.56 (m, 4H). [α]=43.2° c.=1.0 g/100 mL MeOH

### Preparation 19 and 20

### (3S,4R)-3-cyclopropyl-4-methyl-2-oxopyrrolidine-3-carbonitrile and (3R,4R)-3-cyclopropyl-4-methyl-2-oxopyrrolidine-3-carbonitrile

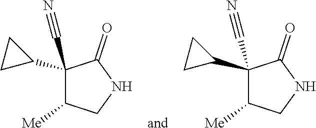 [SMILES: C[C@H]1CNC(=O)[C@@]1(C#N)C1CC1 ; C[C@H]1CNC(=O)[C@]1(C#N)C1CC1]

(4R)-3-Cyclopropyl-4-methyl-2-oxopyrrolidine-3-carbonitrile (Preparation 9, 18.50 g, 112.66 mmol) was separated by SFC (Chiralpak IC, 250×30 mm, 5 Qim; eluting with 30% MeOH (0.1% NH₄OH) in CO₂) to provide Peak 1, Preparation 19, (3S,4R)-3-cyclopropyl-4-methyl-2-oxopyrrolidine-3-carbonitrile as a white solid (6.90 g, 37%). LCMS m/z=165 [M+H]⁺; ¹HNMR (400 MHz, CDCl₃) δ: 6.81 (s, 1H), 3.54 (ddd, 1H), 3.10 (t, 1H), 3.05-2.91 (m, 1H), 1.32 (d, 3H), 1.09-0.95 (m, 1H), 0.81-0.58 (m, 4H)

### Further Elution Provided

Peak 2, Preparation 20, (3R,4R)-3-cyclopropyl-4-methyl-2-oxopyrrolidine-3-carbonitrile as a yellow solid (10.50 g, 57%). LCMS m/z=165 [M+H]⁺; ¹HNMR (400 MHz, CDCl₃) δ: 6.81 (s, 1H), 3.53 (ddd, 1H), 3.10 (dd, 1H), 2.57 (dd, 1H), 1.34 (t, 3H), 1.17-0.97 (m, 1H), 0.83-0.54 (m, 4H).

### Preparations 21 and 22

### (3R,4S)-3-cyclopropyl-4-methyl-2-oxopyrrolidine-3-carbonitrile and (3S,4S)-3-cyclopropyl-4-methyl-2-oxopyrrolidine-3-carbonitrile

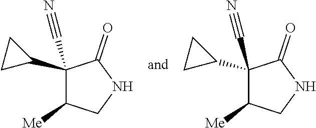 [SMILES: C[C@@H]1CNC(=O)[C@]1(C#N)C1CC1 ; C[C@@H]1CNC(=O)[C@@]1(C#N)C1CC1]

(4S)-3-Cyclopropyl-4-methyl-2-oxopyrrolidine-3-carbonitrile (Preparation 10, 16.50 g) was separated by SFC (Chiralpak IC, 250×30 mm, 5 Qim; eluting with 30% MeOH (0.1% NH₄OH) in CO₂) to afford the title compounds.

Peak 1, Preparation 21; (3R,4S)-3-cyclopropyl-4-methyl-2-oxopyrrolidine-3-carbonitrile as a yellow solid (8.90 g). LCMS m/z=165 [M+H]⁺; ¹HNMR (400 MHz, CDCl₃) δ: 7.21 (s, 1H), 3.56-3.44 (m, 1H), 3.07 (dd, 1H), 2.55 (dt, 1H), 1.33 (d, 3H), 1.08 (dt, 1H), 0.78-0.51 (m, 4H)

Peak 2, Preparation 22; (3S,4S)-3-cyclopropyl-4-methyl-2-oxopyrrolidine-3-carbonitrile (7.30 g) as yellow solid. LCMS m/z=165 [M+H]⁺; ¹HNMR (400 MHz, CDCl₃) δ: 7.20-6.78 (m, 1H), 3.64-3.38 (m, 1H), 3.10 (t, 1H), 3.05-2.88 (m, 1H), 1.40-1.14 (m, 3H), 1.03 (tt, 1H), 0.83-0.53 (m, 4H).

### Preparation 23

### (3S,4S)-4-methyl-3-(oxetan-3-yl)-2-oxopyrrolidine-3-carbonitrile and

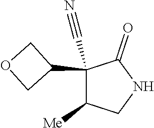 [SMILES: C[C@@H]1CNC(=O)[C@]1(C#N)C1COC1]

(4S)-4-Methyl-3-(oxetan-3-yl)-2-oxopyrrolidine-3-carbonitrile (Preparation 11) was separated by SFC (Chiralpak IC, 250×30 mm, 5 □m; eluting with 30% MeOH (0.1% NH₄OH) in CO₂) to give:

Peak 1, (3R,4S)-4-methyl-3-(oxetan-3-yl)-2-oxopyrrolidine-3-carbonitrile as a white solid (16.4 g, 51.3%).

Peak 2, Preparation 23; (3S,4S)-4-methyl-3-(oxetan-3-yl)-2-oxopyrrolidine-3-carbonitrile as a white solid (13.20 g, 41.3%). LCMS m/z=181 [M+H]⁺; ¹HNMR (400 MHz, CDCl3) δ: 6.87 (s, 1H), 5.07 (t, 1H), 4.97-4.62 (m, 3H), 3.71-3.32 (m, 2H), 3.14-2.77 (m, 2H), 1.04 (d, 3H)

### Preparation 24

### tert-butyl (S)-4-ethyl-1,2,3-oxathiazolidine-3-carboxylate 2,2-dioxide

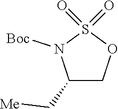 [SMILES: CC[C@H]1COS(=O)(=O)N1C(=O)OC(C)(C)C]

Part 1. Boc₂O (367.3 g, 1.68 mol) was added dropwise to a mixture of (S)-2-aminobutan-1-ol (150 g, 1.68 mol) in DCM (1 L) at 0-10° C. and the resulting mixture stirred at 30° C. for 6 h. The mixture was concentrated under vacuum to give tert-butyl (S)-(1-hydroxybutan-2-yl)carbamate (318.5 g, 100%) as light yellow oil which was used in Part 2 without further purification.

Part 2. A solution of SOC₂ (330 g, 2.77 mol) in MeCN (1000 mL) was cooled to −35° C. and tert-butyl (R)-(1-hydroxybutan-2-yl)carbamate (Part 1, 210 g, 1.11 mol) in MeCN (500 mL) added and the mixture stirred at −35° C. for 1 h. To this mixture was then added pyridine (438.86 g, 5.55 mol) dropwise and the mixture was warmed to 30° C. slowly and stirred for another 8 h. The mixture poured into ice water (1 L) and extracted with EtOAc (2×1 L). The combined organics were washed with saturated aq. NaHCO₃ (500 mL), dried (MgSO₄) and evaporated to dryness in vacuo to afford tert-butyl (4S)-4-ethyl-1,2,3-oxathiazolidine-3-carboxylate 2-oxide as a brown oil (250 g, 96%) which was used in Part 3 without further purification.

Part 3. tert-butyl (4S)-4-ethyl-1,2,3-oxathiazolidine-3-carboxylate 2-oxide (Part 2, 250 g, 1.06 mol) was dissolved in MeCN (1 L) and H₂O (1 L) at 20° C. To this was added RuCl₃·3H₂O (833 mg, 3.19 mmol) was added at 0° C. followed by NaIO₄ (340.9 g, 1.59 mol) in small portions and the resulting mixture was stirred at 15° C. for 2 h. The reaction mixture was filtered and the filtrate was extracted with EtOAc (2×800 mL). The combined organics were washed with saturated aq. NaHSO₃ (500 mL) and aq. NaHCO₃ (500 mL), dried (MgSO₄) and concentrated. The residue was triturated with n-Hep/EtOH (20:1, 220 mL) and the solid collected by filtration to afford tert-butyl (S)-4-ethyl-1,2,3-oxathiazolidine-3-carboxylate 2,2-dioxide as a white solid (150 g, 56%). ¹HNMR (400 MHz, CDCl₃) δ: ppm 4.66 (dd, 1H), 4.44-4.13 (m, 2H), 1.91 (ddd, 2H), 1.57 (s, 9H), 1.01 (t, 3H).

### Preparation 25

### (5S)-3-cyclopropyl-5-ethyl-2-oxopyrrolidine-3-carbonitrile

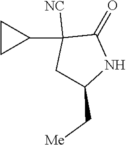 [SMILES: CC[C@H]1CC(C#N)(C2CC2)C(=O)N1]

Part 1. Cs₂CO₃ (128 g, 391 mmol) and TBAB (16.84 g, 52.2 mmol) were added at 10° C. to a solution of ethyl 2-cyano-2-cyclopropylacetate (Preparation 4, 40 g, 261 mmol) in MeCN (500 mL) and the mixture stirred for 30 min. To this was added tert-butyl (S)-4-ethyl-1,2,3-oxathiazolidine-3-carboxylate 2,2-dioxide (Preparation 24, 65.62 g, 261 mmol) and the resulting mixture stirred at 30° C. for 10 h. The reaction mixture was evaporated to dryness in vacuo and the residue partitioned between H₂O (200 mL) and EtOAc (500 mL). The pH was adjusted to pH-5 with 1 M HCl at 10° C., then extracted with EtOAc (500 mL and 2×200 mL). The combined organics were washed with saturated aq. NaHCO₃ (200 mL), dried (MgSO₄) and evaporated to dryness to afford ethyl (4S)-4-((tert-butoxycarbonyl)amino)-2-cyano-2-cyclopropylhexanoate (solution in EtOAc, 900 mL) which was used immediately in Part 2.

Part 3. TsOH·H₂O (99.33 g, 522 mmol) was added to a solution of ethyl (4S)-4-((tert-butoxycarbonyl)amino)-2-cyano-2-cyclopropylhexanoate (Part 1, 84.71 g, 261 mmol) in EtOAc (900 mL) at rt and the mixture was heated at 50° C. for 1 h. The reaction mixture was cooled to rt and concentrated under vacuum. The residue was dissolved in MeCN (800 mL) and K₂CO₃ (108.27 g, 783 mmol) added in small portions and the resulting mixture stirred at 50° C. for 5 hr. The reaction mixture was cooled to room temperature and evaporated to dryness in vacuo. The residue was partitioned between H₂O (500 mL) and EtOAc (500 mL). The combined organics were dried (MgSO₄) and evaporated to dryness in vacuo. The residue was purified by column chromatography (SiO2, 0-50% EtOAc/PE) to give (5S)-3-cyclopropyl-5-ethyl-2-oxopyrrolidine-3-carbonitrile as a white solid (28.2 g, 61%). ¹HNMR (400 MHz, CDCl₃) δ: 6.65 (d, 1H), 3.85-3.47 (m, 1H), 2.70 (dd, 1H), 2.48 (dd, 1H), 2.28-2.12 (m, 1H), 1.82 (dd, 1H), 1.68 (ddd, 1H), 1.62-1.48 (m, 1H), 1.30-1.16 (m, 1H), 1.00 (td, 3H), 0.85-0.55 (m, 4H).

### Preparation 26 and 27

### (3R,5S)-3-cyclopropyl-5-ethyl-2-oxopyrrolidine-3-carbonitrile and (3S,5S)-3-cyclopropyl-5-ethyl-2-oxopyrrolidine-3-carbonitrile

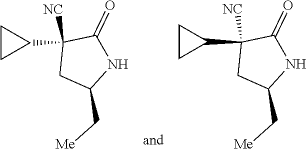 [SMILES: CC[C@H]1C[C@](C#N)(C2CC2)C(=O)N1 ; CC[C@H]1C[C@@](C#N)(C2CC2)C(=O)N1]

(5S)-3-cyclopropyl-5-ethyl-2-oxopyrrolidine-3-carbonitrile (Preparation 25, 15 g, 84.2 mmol) was separated by SFC (ChiralPak IC, 150×4.6 mm, 3 Dim; 5-40% MeOH (+0.1% NH₄OH) in CO₂) to afford the title compounds as off-white solids.

Peak 1, Preparation 26 (4.30 g, 29%); (3R,5S)-3-cyclopropyl-5-ethyl-2-oxopyrrolidine-3-carbonitrile. LCMS m/z=179 [M+H]⁺; ¹HNMR (400 MHz, CDCl₃) δ: 6.58 (s, 1H), 3.74-3.44 (m, 1H), 2.48 (dd, 1H), 2.26 (dd, 1H), 1.76-1.65 (m, 1H), 1.60 (dd, 1H), 1.33-1.16 (m, 1H), 1.00 (t, 3H), 0.83-0.51 (m, 4H). [α]=−15.4° (c=1.1 g/100 mL, MeOH).

Peak 2, Preparation 27 (7.60 g, 51%); (3S,5S)-3-cyclopropyl-5-ethyl-2-oxopyrrolidine-3-carbonitrile. LCMS m/z=179 [M+H]⁺; ¹HNMR (400 MHz, CDCl₃) δ: 6.58 (s, 1H), 3.85-3.56 (m, 1H), 2.70 (dd, 1H), 1.82 (dd, 1H), 1.69 (d, 1H), 1.65-1.48 (m, 1H), 1.22 (tt, 1H), 1.00 (t, 3H), 0.88-0.54 (m, 4H). [α]=38.0° (c=1.0 g/100 mL, MeOH).

### Preparation 28

### tert-butyl (R)-4-ethyl-1,2,3-oxathiazolidine-3-carboxylate 2,2-dioxide

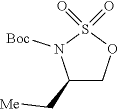 [SMILES: CC[C@@H]1COS(=O)(=O)N1C(=O)OC(C)(C)C]

Part 1. Boc₂O (244.85 g, 1.12 mol) was added dropwise to a mixture of (R)-2-aminobutan-1-ol (100 g, 1.12 mol) in THF (1 L) at 0-10° C. and the resulting mixture stirred at 30° C. for 6 h. The mixture was concentrated under vacuum to give tert-butyl (R)-(1-hydroxybutan-2-yl)carbamate (212.32 g, 100%) as light yellow oil which was used in Part 2 without further purification.

Part 2. A solution of SOC₂ (330 g, 2.77 mol) in MeCN (1000 mL) was cooled to −35° C. and tert-butyl (R)-(1-hydroxybutan-2-yl)carbamate (Part 1, 210 g, 1.11 mol) in MeCN (500 mL) added and the mixture stirred at −35° C. for 1 h. To this mixture was then added pyridine (438.86 g, 5.55 mol) dropwise and the mixture was warmed to 30° C. slowly and stirred for another 5 h. The mixture poured into ice water (1 L) and extracted with EtOAc (2×1 L). The combined organics were washed with saturated aq. NaHCO₃ (500 mL), dried (MgSO₄) and evaporated to dryness in vacuo to afford tert-butyl (4R)-4-ethyl-1,2,3-oxathiazolidine-3-carboxylate 2-oxide as a brown oil (230 g, 88%) which was used in Part 3 without further purification.

Part 3. tert-butyl (4R)-4-ethyl-1,2,3-oxathiazolidine-3-carboxylate 2-oxide (Part 2, 230 g, 977 mmol) was dissolved in MeCN (1 L) and H₂O (1 L) at 20° C. To this was added RuCl₃·3H₂O (767 mg, 2.93 mmol) was added at 0° C. followed by NaIO₄ (313.6 g, 1466 mmol) in small portions and the resulting mixture was stirred at 20° C. for 1 h. The reaction mixture was filtered and the filtrate was extracted with EtOAc (2×800 mL). The combined organics were washed with saturated aq. NaHSO₃ (500 mL) and aq. NaHCO₃ (500 mL), dried (MgSO₄) and concentrated. The residue was triturated with n-Hep/EtOH (20:1, 220 mL) and the solid collected by filtration to afford tert-butyl (R)-4-ethyl-1,2,3-oxathiazolidine-3-carboxylate 2,2-dioxide as a white solid (180 g, 73%). ¹HNMR (400 MHz, CDCl₃) δ: MHz, CDCl₃) δ: ppm 4.66 (dd, 1H), 4.44-4.13 (m, 2H), 1.91 (ddd, 2H), 1.57 (s, 9H), 1.01 (t, 3H).

### Preparation 29

### (5R)-3-cyclopropyl-5-ethyl-2-oxopyrrolidine-3-carbonitrile

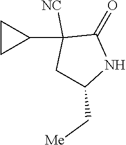 [SMILES: CC[C@@H]1CC(C#N)(C2CC2)C(=O)N1]

Part 1. Cs₂CO₃ (128 g, 391 mmol) and TBAB (16.84 g, 52.2 mmol) were added at 10° C. to a solution of ethyl 2-cyano-2-cyclopropylacetate (Preparation 4, 40 g, 261 mmol) in MeCN (500 mL) and the mixture stirred for 30 min. To this was added tert-butyl (R)-4-ethyl-1,2,3-oxathiazolidine-3-carboxylate 2,2-dioxide (Preparation 28, 65.62 g, 261 mmol) and the resulting mixture stirred at 30° C. for 10 h. The reaction mixture was evaporated to dryness in vacuo and the residue partitioned between H₂O (200 mL) and EtOAc (500 mL). The pH was adjusted to pH-5 with 1 M HCl at 10° C., then extracted with EtOAc (500 mL and 2×200 mL). The combined organics were washed with saturated aq. NaHCO₃ (200 mL), dried (MgSO₄) to afford ethyl (4R)-4-((tert-butoxycarbonyl)amino)-2-cyano-2-cyclopropylhexanoate (solution in EtOAc, 900 mL) which was used immediately in Part 2.

Part 2. TsOH·H₂O (99.33 g, 522 mmol) was added to a solution of ethyl (4R)-4-((tert-butoxycarbonyl)amino)-2-cyano-2-cyclopropylhexanoate (Part 1, 84.71 g, 261 mmol) in EtOAc (900 mL) at rt and the mixture was heated at 50° C. for 1 h. The reaction mixture was cooled to rt and concentrated under vacuum. The residue was dissolved in MeCN (800 mL) and K₂CO₃ (108.27 g, 783 mmol) added in small portions and the resulting mixture stirred at 50° C. for 5 hr. The reaction mixture was cooled to room temperature and evaporated to dryness in vacuo. The residue was partitioned between H₂O (500 mL) and EtOAc (500 mL). The combined organics were dried (MgSO₄) and evaporated to dryness in vacuo. The residue was purified by column chromatography (SiO2, 0-50% EtOAc/PE) to give (5R)-3-cyclopropyl-5-ethyl-2-oxopyrrolidine-3-carbonitrile as a white solid (32.8 g, 70%). ¹HNMR (400 MHz, CDCl₃) δ: 6.65 (d, 1H), 3.85-3.47 (m, 1H), 2.70 (dd, 1H), 2.48 (dd, 1H), 2.28-2.12 (m, 1H), 1.82 (dd, 1H), 1.68 (ddd, 1H), 1.62-1.48 (m, 1H), 1.30-1.16 (m, 1H), 1.00 (td, 3H), 0.85-0.55 (m, 4H).

### Preparation 30 and 31

### (3S,5R)-3-cyclopropyl-5-ethyl-2-oxopyrrolidine-3-carbonitrile and (3R,5R)-3-cyclopropyl-5-ethyl-2-oxopyrrolidine-3-carbonitrile

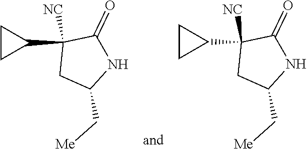 [SMILES: CC[C@@H]1C[C@@](C#N)(C2CC2)C(=O)N1 ; CC[C@@H]1C[C@](C#N)(C2CC2)C(=O)N1]

(5R)-3-cyclopropyl-5-ethyl-2-oxopyrrolidine-3-carbonitrile (Preparation 29, 20 g, 112.21 mmol) was separated by SFC (ChiralPak IC, 150×4.6 mm, 3 □m; 5-40% MeOH (+0.1% NH₄OH) in CO₂) to afford the title compounds as light-yellow solids.

Peak 1, Preparation 30 (5.85 g, 39%); (3S,5R)-3-cyclopropyl-5-ethyl-2-oxopyrrolidine-3-carbonitrile. LCMS m/z=179 [M+H]⁺; ¹HNMR (400 MHz, CDCl₃) δ: 6.58 (s, 1H), 3.74-3.44 (m, 1H), 2.48 (dd, 1H), 2.26 (dd, 1H), 1.76-1.65 (m, 1H), 1.60 (dd, 1H), 1.33-1.16 (m, 1H), 1.00 (t, 3H), 0.83-0.51 (m, 4H). [α]=14.7° (c=1.1 g/100 mL, MeOH).

Peak 2, Preparation 31 (7.85 g, 52%); (3R,5R)-3-cyclopropyl-5-ethyl-2-oxopyrrolidine-3-carbonitrile. LCMS m/z=179 [M+H]⁺; ¹HNMR (400 MHz, CDCl₃) δ: 6.58 (s, 1H), 3.85-3.56 (m, 1H), 2.70 (dd, 1H), 1.82 (dd, 1H), 1.69 (d, 1H), 1.65-1.48 (m, 1H), 1.22 (tt, 1H), 1.00 (t, 3H), 0.88-0.54 (m, 4H). [α]=−39.0° (c=1.0 g/100 mL, MeOH).

### Preparation 32

### tert-butyl (R)-5-ethyl-1,2,3-oxathiazolidine-3-carboxylate 2,2-dioxide

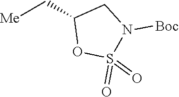 [SMILES: CC[C@@H]1CN(C(=O)OC(C)(C)C)S(=O)(=O)O1]

Part 1. A mixture of NHBn₂ (126.27 g, 640 mmol) and (R)-2-ethyloxirane (60.0 g, 832 mmol) in EtOH (600 mL) was stirred at 50° C. for 20 h. The mixture was concentrated under vacuum to give (R)-1-(dibenzylamino)butan-2-ol as a yellow oil (172.43 g, 100%) which was used in Part 2 without further purification.

Part 2. To a solution of (R)-1-(dibenzylamino)butan-2-ol (Part 1, 172.43 g, 640 mmol) in MeOH (800 mL) was added Pd(OH)₂/C (20 g, 20% purity) under N₂. The resulting mixture was degassed and purged with H₂ (3×) and the reaction mixture stirred under H₂ (1 atm) at 50° C. for 5 h. After filtration, the filtrate was concentrated to afford (R)-1-aminobutan-2-ol as a colourless oil (57.46 g, ~100%) which was used without further purification in Part 3.

Part 3. To a solution of (R)-1-aminobutan-2-ol (57.46 g, 640 mmol) in THE was added Boc₂O (140.74 g, 645 mmol) and the mixture stirred at 30° C. for 6 h. The mixture was concentrated to afford tert-butyl (R)-(2-hydroxybutyl)carbamate as a colourless oil (120.50 g, 99%) which was used without further purification in Part 4.

Part 4. tert-butyl (R)-(2-hydroxybutyl)carbamate (Part 3, 110 g, 581 mmol) in MeCN (500 mL) was added to a solution of SOCl₂ (173 g, 1453 mmol) in MeCN (1000 mL) at −40° C. After the addition was complete the reaction mixture was stirred at −25° C. for 1 h. Pyridine (230 g, 2.90 mol) was added and the mixture slowly warmed to 30° C. and stirred for another 10 h. The mixture poured into ice water (1 L) and extracted with EtOAc (2×1 L). The combined organic layers were washed with saturated aq. NaHCO₃ (500 mL), dried (MgSO₄) and concentrated to afford tert-butyl (5R)-5-ethyl-1,2,3-oxathiazolidine-3-carboxylate 2-oxide as a brown oil (125 g, 91%) as a brown oil.

Part 5. tert-butyl (5R)-5-ethyl-1,2,3-oxathiazolidine-3-carboxylate 2-oxide (Part 4, 125 g, 531 mmol) was dissolved in MeCN (1 L) and H₂O (1 L) at 20° C. RuCl₃·3H₂O (694.41 mg, 2.66 mmol) was added followed by NaIO₄ (170.44 g, 797 mmol) in small portions. After the addition, the mixture was stirred at 20° C. for 2 h. The reaction mixture was filtered and the filtrate was extracted with EtOAc (3×500 mL). The combined organics were washed with saturated aq. NaHSO₃ (500 mL) and aq. NaHCO₃ (500 mL), dried (MgSO₄) and evaporated to dryness in vacuo. The residue was triturated with n-Hep/EtOH (10:1, 200 mL) and filtered to afford tert-butyl (R)-5-ethyl-1,2,3-oxathiazolidine-3-carboxylate 2,2-dioxide as a white solid (118 g, 88%). ¹HNMR (400 MHz, CDCl₃) δ: 4.78 (dd, 1H), 4.07 (dd, 1H), 3.68 (t, 1H), 1.97 (dd, 1H), 1.89-1.71 (m, 1H), 1.57 (s, 9H), 1.09 (t, 3H).

### Preparation 33

### (4R)-3-cyclopropyl-4-ethyl-2-oxopyrrolidine-3-carbonitrile

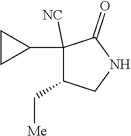 [SMILES: CC[C@H]1CNC(=O)C1(C#N)C1CC1]

The title compound was prepared as a yellow oil (43 g, 74%) from tert-butyl (R)-5-ethyl-1,2,3-oxathiazolidine-3-carboxylate 2,2-dioxide (Preparation 32) using an analogous method to that described for (Preparation 29). ¹HNMR (400 MHz, CDCl₃) δ: 0.31 (s, 1H), 7.23 (s, 1H), 3.55 (ddt, 1H), 3.19-2.95 (m, 1H), 2.89-2.65 (m, 1H), 2.36 (dtd, 1H), 1.99-1.79 (m, 1H), 1.69-1.53 (m, 1H), 1.09-0.87 (m, 4H), 0.80-0.47 (m, 4H).

### Preparation 34 and 35

### (3S,4R)-3-cyclopropyl-4-ethyl-2-oxopyrrolidine-3-carbonitrile and (3R,4R)-3-cyclopropyl-4-ethyl-2-oxopyrrolidine-3-carbonitrile

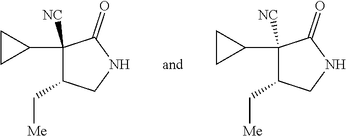 [SMILES: CC[C@H]1CNC(=O)[C@@]1(C#N)C1CC1 ; CC[C@H]1CNC(=O)[C@]1(C#N)C1CC1]

(4R)-3-cyclopropyl-4-ethyl-2-oxopyrrolidine-3-carbonitrile (Preparation 33) was separated by SFC (ChiralPak IC, 150×4.6 mm, 3 □m; 30% MeOH (+0.1% NH₄OH) in CO₂) to afford the title compounds as light-yellow solids.

Peak 1, Preparation 34 (8.60 g, 43%); (3S,4R)-3-cyclopropyl-4-ethyl-2-oxopyrrolidine-3-carbonitrile. LCMS m/z=179 [M+H]⁺; ¹HNMR (400 MHz, CDCl₃) δ: 7.06 (s, 1H), 3.56 (ddd, 1H), 3.09 (t, 1H), 2.87-2.65 (m, 1H), 2.04-1.77 (m, 1H), 1.79-1.48 (m, 2H), 1.22-0.83 (m, 4H), 0.87-0.43 (m, 4H). [α]=11.1° (c=1.0 g/100 mL, MeOH).

Peak 2, Preparation 35 (9.40 g, 47%); (3R,4R)-3-cyclopropyl-4-ethyl-2-oxopyrrolidine-3-carbonitrile. LCMS m/z=179 [M+H]⁺; ¹HNMR (400 MHz, CDCl₃) δ: 7.07 (s, 1H), 3.55 (ddd, 1H), 3.15 (dd, 1H), 2.44-2.20 (m, 1H), 1.90 (ddd, 1H), 1.79-1.53 (m, 2H), 1.22-0.91 (m, 4H), 0.82-0.41 (m, 4H). [α]=66.1° (c=1.1 g/100 mL, MeOH).

### Preparation 36

### tert-butyl (S)-5-ethyl-1,2,3-oxathiazolidine-3-carboxylate 2,2-dioxide

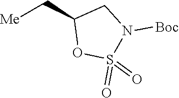 [SMILES: CC[C@H]1CN(C(=O)OC(C)(C)C)S(=O)(=O)O1]

The title compound was prepared as a white solid (120 g, 90%) from (S)-2-ethyloxirane using an analogous 5-part procedure as described for Preparation 32. ¹HNMR (400 MHz, CDCl₃) δ: 4.78 (dd, 1H), 4.07 (dd, 1H), 3.68 (t, 1H), 1.97 (dd, 1H), 1.89-1.71 (m, 1H), 1.57 (s, 9H), 1.09 (t, 3H).

### Preparation 37

### (4S)-3-cyclopropyl-4-ethyl-2-oxopyrrolidine-3-carbonitrile

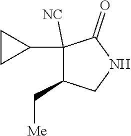 [SMILES: CC[C@@H]1CNC(=O)C1(C#N)C1CC1]

The title compound was prepared as a yellow oil (42.1 g, 72%) from tert-butyl (S)-5-ethyl-1,2,3-oxathiazolidine-3-carboxylate 2,2-dioxide (Preparation 36) using an analogous 2-Part procedure as described for Preparation 29. ¹HNMR (400 MHz, CDCl₃) δ: 7.31 (s, 1H), 7.23 (s, 1H), 3.55 (ddt, 1H), 3.19-2.95 (m, 1H), 2.89-2.65 (m, 1H), 2.36 (dtd, 1H), 1.99-1.79 (m, 1H), 1.69-1.53 (m, 1H), 1.09-0.87 (m, 4H), 0.80-0.47 (m, 4H).

### Preparation 38 and 39

### (3S,4S)-3-cyclopropyl-4-ethyl-2-oxopyrrolidine-3-carbonitrile and (3R,4S)-3-cyclopropyl-4-ethyl-2-oxopyrrolidine-3-carbonitrile

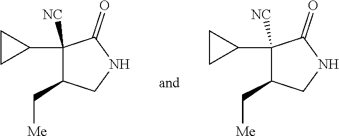 [SMILES: CC[C@@H]1CNC(=O)[C@@]1(C#N)C1CC1 ; CC[C@@H]1CNC(=O)[C@]1(C#N)C1CC1]

(4S)-3-cyclopropyl-4-ethyl-2-oxopyrrolidine-3-carbonitrile (Preparation 37) was separated by SFC (ChiralPak IC, 150×4.6 mm, 3 Dim; 30% MeOH (+0.1% NH₄OH) in CO₂) to afford the title compounds as yellow solids.

Peak 1, Preparation 38 (9.50 g, 47%); (3S,4S)-3-cyclopropyl-4-ethyl-2-oxopyrrolidine-3-carbonitrile. LCMS m/z=179 [M+H]⁺; ¹HNMR (400 MHz, CDCl₃) δ: 7.07 (s, 1H), 3.55 (ddd, J=1H), 3.15 (dd, 1H), 2.44-2.20 (m, 1H), 1.90 (ddd, 1H), 1.79-1.53 (m, 2H), 1.22-0.91 (m, 4H), 0.82-0.41 (m, 4H). [α]=−67.7° (c=1.0 g/100 mL, MeOH).

Peak 2, Preparation 39 (8.80 g, 44%); (3R,4S)-3-cyclopropyl-4-ethyl-2-oxopyrrolidine-3-carbonitrile. LCMS m/z=179 [M+H]⁺; ¹HNMR (400 MHz, CDCl₃) δ: 7.06 (s, 1H), 3.56 (ddd, 1H), 3.09 (t, 1H), 2.87-2.65 (m, 1H), 2.04-1.77 (m, 1H), 1.79-1.48 (m, 2H), 1.22-0.83 (m, 4H), 0.87-0.43 (m, 4H). [α]=−11.2° (c=0.97 g/100 mL, MeOH).

### Preparation 40

### (R)-3-cyclopropyl-1-(4-methoxybenzyl)-2-oxopyrrolidine-3-carboxylic acid

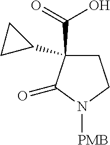

Part A: To a solution of (S)-3-cyclopropyl-2-oxopyrrolidine-3-carbonitrile (Example 1D, WO2016 0159773, 2.0 g, 13.3 mmol) in DMF (15 mL) was added NaH (639 mg, 16.0 mmol, 60% purity) at 0° C. and the mixture stirred for 30 min. PMBCl (2.3 g, 14.7 mmol) was added and the mixture stirred at 25° C. for 1 h. The mixture was diluted with water (20 mL) and extracted with EtOAc (3×25 mL). The combined organics were washed with brine (60 mL), dried (Na₂SO₄) and concentrated to give (S)-3-cyclopropyl-1-(4-methoxybenzyl)-2-oxopyrrolidine-3-carbonitrile as an orange oil (4.0 g, crude.

Part B: To a solution of (S)-3-cyclopropyl-1-(4-methoxybenzyl)-2-oxopyrrolidine-3-carbonitrile (Part A, 4.0 g, 14.80 mmol) in DMF (2 mL) was added NaOH (6 M, 30 mL) and the mixture was stirred at 50° C. for 64 h. The mixture was adjusted to pH 5-6 with 4 M HCl, diluted with water (20 mL) and extracted with EtOAc (3×15 mL). The combined organics were washed with brine (30 mL), dried (Na₂SO₄) and evaporated under reduced pressure to give (R)-3-cyclopropyl-1-(4-methoxybenzyl)-2-oxopyrrolidine-3-carboxylic acid as a white solid (4.0 g, crude). ¹H NMR (500 MHz, CDCl₃) δ: 7.14 (d, 2H), 6.86 (d, 2H), 4.50 (d, 1H), 4.29 (d, 1H), 3.80 (s, 3H), 3.20-3.16 (m, 2H), 2.48-2.41 (m, 1H), 1.92-1.89 (m, 1H), 1.39-1.36 (m, 1H), 0.54-0.48 (m, 3H), 0.30-0.22 (m, 1H).

### Preparation 41

### (R)-3-cyclopropyl-N-methoxy-1-(4-methoxybenzyl)-N-methyl-2-oxopyrrolidine-3-carboxamide

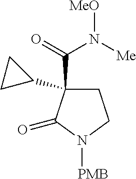

A solution of (R)-3-cyclopropyl-1-(4-methoxybenzyl)-2-oxopyrrolidine-3-carboxylic acid (Preparation 40, 3.5 g, 12.1 mmol), DIPEA (4.7 g, 36.3 mmol), HOBt (2.0 g, 14.5 mmol), EDCI (5.1 g, 26.6 mmol) and N,O-dimethylhydroxylamine hydrochloride (3.0 g, 30.2 mmol) in DCM (70 mL) was stirred at 25° C. for 16 h. The mixture was diluted with water (70 mL) and extracted with EtOAc (3×50 mL). The combined organic phase was washed with brine (60 mL), dried (Na₂SO₄) and concentrated in vacuo. The residue was purified by column chromatography (SiO₂, 0-50% EtOAc/PE) to give (R)-3-cyclopropyl-N-methoxy-1-(4-methoxybenzyl)-N-methyl-2-oxopyrrolidine-3-carboxamide as a colourless oil (3.3 g, 82%). LCMS m/z=333.2 [M+H]⁺

### Preparation 42

### (S)-3-cyclopropyl-1-(4-methoxybenzyl)-2-oxopyrrolidine-3-carbaldehyde

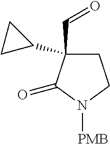

To a solution of (R)-3-cyclopropyl-N-methoxy-1-(4-methoxybenzyl)-N-methyl-2-oxopyrrolidine-3-carboxamide (Preparation 41, 3.8 g, 11.34 mmol) in DCM (30 mL) was added DIBAL-H (1 M, 22.7 mL) at −78° C. under N₂ and the mixture stirred for 2 h. The mixture was diluted with sat. aq. NH₄Cl (50 mL) and extracted with EtOAc (3×40 mL). The combined organics were washed with brine (80 mL), dried (Na₂SO₄) and concentrated in vacuo. The residue was purified by column chromatography (SiO₂, 0-20% EtOAc/PE) to give (S)-3-cyclopropyl-1-(4-methoxybenzyl)-2-oxopyrrolidine-3-carbaldehyde as a colourless oil (2.1 g, 67%). ¹H NMR (400 MHz, CDCl₃) δ: 9.80 (br s, 1H), 7.14 (d, 2H), 6.86 (d, 2H), 4.44 (d, 1H), 4.30 (d, 1H), 3.81 (s, 3H), 3.14 (t, 2H), 2.46-2.41 (m, 1H), 1.55-1.51 (m, 1H), 1.27-1.25 (m, 1H), 0.74-0.71 (m, 1H), 0.60-0.57 (m, 2H), 0.23-0.12 (m, 1H).

### Preparation 43

### (R)-3-cyclopropyl-3-(difluoromethyl)-1-(4-methoxybenzyl)pyrrolidin-2-one

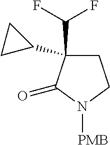

To a solution of (S)-3-cyclopropyl-1-(4-methoxybenzyl)-2-oxopyrrolidine-3-carbaldehyde (Preparation 42, 500 mg, 1.83 mmol) in DCM (5 mL) was added DAST (590 mg, 0.5 mL) at 0° C. The mixture was warmed to 25° C. and stirred for 16 h. The mixture was diluted with water (10 mL) and extracted with EtOAc (3×15 mL). The combined organics were washed with brine (30 mL), dried (Na₂SO₄) and concentrated in vacuo. The residue was purified by column chromatography (SiO₂, 0-20% EtOAc/PE) to give (R)-3-cyclopropyl-3-(difluoromethyl)-1-(4-methoxybenzyl)pyrrolidin-2-one as a colourless oil (347 mg, 64%). LCMS m/z=296.2 [M+H]⁺

### Preparation 44

### (R)-3-cyclopropyl-3-(difluoromethyl)pyrrolidin-2-one

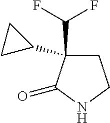 [SMILES: O=C1NCC[C@@]1(C(F)F)C1CC1]

To a solution of (R)-3-cyclopropyl-3-(difluoromethyl)-1-(4-methoxybenzyl)pyrrolidin-2-one (Preparation 43, 347 mg, 1.17 mmol) in MeCN (10 mL) and H₂O (1 mL) was added ceric ammonium nitrate (1.9 g, 3.52 mmol) and the reaction stirred at 25° C. for 16 h. The mixture was diluted with water (10 mL) and extracted with EtOAc (3×15 mL). The combined organic phase was washed with brine (30 mL), dried over anhydrous Na₂SO₄ filtered and concentrated. The crude was purified by prep-TLC (33% EtOAc/PE) to give (R)-3-cyclopropyl-3-(difluoromethyl)pyrrolidin-2-one as a white solid (130 mg, 63%). ¹H NMR (500 MHz, CDCl₃) δ: 6.11-5.88 (m, 2H), 3.37-3.33 (m, 2H), 2.54-2.47 (m, 1H), 1.86-1.83 (m, 1H), 1.09-1.04 (m, 1H), 0.70-0.66 (m, 1H), 0.64-0.57 (m, 1H), 0.50-0.48 (m, 1H), 0.32-0.30 (m, 1H).

### Preparation 45

### (S)-3-cyclopropyl-3-(hydroxymethyl)-1-(4-methoxybenzyl)pyrrolidin-2-one

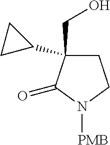

To a solution of (S)-3-cyclopropyl-1-(4-methoxybenzyl)-2-oxopyrrolidine-3-carbaldehyde (Preparation 42, 800 mg, 2.93 mmol) in MeOH (10 mL) was slowly added NaBH₄ (554 mg, 14.63 mmol) at 0° C. and the reaction then stirred at 40° C. for 16 h. The mixture was evaporated under reduced pressure and the residue diluted with water (10 mL) and extracted with EtOAc (3×15 mL). The combined organics were washed with brine (30 mL), dried (Na₂SO₄) and evaporated under reduced pressure to afford (S)-3-cyclopropyl-3-(hydroxymethyl)-1-(4-methoxybenzyl)pyrrolidin-2-one as a colourless oil (343 mg, 42.6%). LCMS m/z=276.2 [M+H]⁺

### Preparation 46

### (S)-(3-cyclopropyl-1-(4-methoxybenzyl)-2-oxopyrrolidin-3-yl)methyl methanesulfonate

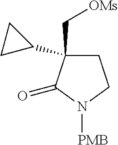

To a solution of (S)-3-cyclopropyl-3-(hydroxymethyl)-1-(4-methoxybenzyl)pyrrolidin-2-one (Preparation 45, 343 mg, 1.25 mmol) in DCM (10 mL) was added TEA (379 mg, 3.74 mmol) and MsCl (214 mg, 1.87 mmol) at 0° C. The mixture was stirred at 25° C. for 3 h then diluted with water (10 mL) and extracted with DCM (3×15 mL). The combined organics were washed with brine (30 mL), dried (Na₂SO₄) and concentrated to give (S)-(3-cyclopropyl-1-(4-methoxybenzyl)-2-oxopyrrolidin-3-yl)methyl methanesulfonate as a yellow oil (453 mg, crude) as yellow oil. LCMS m/z=354.1 [M+H]⁺

### Preparation 47

### (R)-2-(3-cyclopropyl-1-(4-methoxybenzyl)-2-oxopyrrolidin-3-yl)acetonitrile

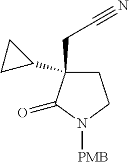

To a solution of (S)-(3-cyclopropyl-1-(4-methoxybenzyl)-2-oxopyrrolidin-3-yl)methyl methanesulfonate (Preparation 46, 453 mg, 1.28 mmol) in DMSO (6 mL) was added NaCN (94.3 mg, 1.92 mmol) and NaI (19.2 mg, 0.128 mmol) and the reaction stirred at 150° C. for 3 h. The cooled mixture was diluted with water (15 mL), extracted with EtOAc (10 mL×3) and the combined organic phase was washed with brine (30 mL), dried (Na₂SO₄) and concentrated. The crude was purified by column chromatography (SiO₂, 0-20% EtOAc/PE) to give (R)-2-(3-cyclopropyl-1-(4-methoxybenzyl)-2-oxopyrrolidin-3-yl)acetonitrile as a colourless oil (285 mg, 78%). LCMS m/z=285.2 [M+H]⁺

### Preparation 48

### (R)-2-(3-cyclopropyl-2-oxopyrrolidin-3-yl)acetonitrile

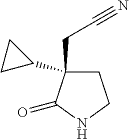 [SMILES: N#CC[C@@]1(C2CC2)CCNC1=O]

(R)-2-(3-Cyclopropyl-2-oxopyrrolidin-3-yl)acetonitrile was obtained as a colourless solid (93.2 mg, 57%) from (R)-2-(3-cyclopropyl-1-(4-methoxybenzyl)-2-oxopyrrolidin-3-yl)acetonitrile (Preparation 47) following the procedure described in Preparation 44. ¹H NMR (500 MHz, CDCl₃) δ: 5.83 (s, 1H), 3.39-3.35 (m, 2H), 2.67 (q, 2H), 2.26-2.21 (m, 1H), 2.13-2.11 (m, 1H), 1.11-1.07 (m, 1H), 0.64-0.53 (m, 1H), 0.52-0.48 (m, 2H), 0.38-0.35 (m, 1H).

### Preparation 49

### 4-chloro-1-tosyl-1H-pyrrolo[2,3-b]pyridine

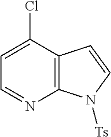 [SMILES: Cc1ccc(S(=O)(=O)n2ccc3c(Cl)ccnc32)cc1]

To a solution of 4-chloro-1H-pyrrolo[2,3-b]pyridine (5.0 g, 32.8 mmol) in DMF (80 mL) was added NaH (1.75 g, 60% purity, 39.3 mmol) at 0° C., the mixture stirred at 20° C. for 30 mins, then TsCl (9.4 g, 49.16 mmol) added. The reaction was stirred at 20° C. for 1 h, quenched with water (30 mL) and extracted with EtOAc (3×20 mL). The combined organics were washed with brine (20 mL), dried (Na₂SO₄) and concentrated in vacuo. The residue was purified by column chromatography (SiO₂, 9-17% EtOAc/PE) to afford 4-chloro-1-tosyl-1H-pyrrolo[2,3-b]pyridine as a white solid (6.1 g, 60.7% yield). ¹H NMR (400 MHz, CDCl₃) δ: 8.29 (d, 1H), 8.04 (d, 2H), 7.75 (d, 1H), 7.26 (d, 2H), 7.17 (d, 1H), 6.68 (d, 1H), 2.36 (s, 3H).

### Preparation 50

### 4-chloro-2-iodo-1-tosyl-1H-pyrrolo[2,3-b]pyridine

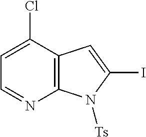 [SMILES: Cc1ccc(S(=O)(=O)n2c(I)cc3c(Cl)ccnc32)cc1]

To a solution of 4-chloro-1-tosyl-1H-pyrrolo[2,3-b]pyridine (Preparation 49, 2.0 g, 6.52 mmol) in THE (50 mL) was added LDA (2 M, 3.91 mL) at −70° C. and the solution stirred for 30 mins. I₂ (2.0 g, 7.82 mmol) was added and the reaction stirred at 20° C. for 1 h. The mixture was quenched with water (30 mL) and extracted with EtOAc (3×20 mL). The combined organic layers were washed with brine (20 mL), dried (Na₂SO₄) and concentrated in vacuo. The crude was purified by column chromatography (SiO2, 9-17% EtOAc/PE) to give 4-chloro-2-iodo-1-tosyl-1H-pyrrolo[2,3-b]pyridine as a white solid (800 mg, 28.4%). ¹H NMR (400 MHz, CDCl₃) δ: 8.27 (d, 1H), 8.09 (d, 2H), 7.28 (d, 2H), 7.15 (d, 1H), 7.10 (s, 1H), 2.38 (s, 3H).

### Preparation 51

### 4-chloro-2-(1-methyl-1H-pyrazol-4-yl)-1-tosyl-1H-pyrrolo[2,3-b]pyridine

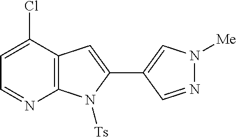 [SMILES: Cc1ccc(S(=O)(=O)n2c(-c3cnn(C)c3)cc3c(Cl)ccnc32)cc1]

To a solution of 4-chloro-2-iodo-1-tosyl-1H-pyrrolo[2,3-b]pyridine (Preparation 50, 500 mg, 1.16 mmol) in dioxane (3 mL) and H₂O (0.3 mL) were added 1-methylpyrazole-4-boronic acid pinacol ester (289.6 mg, 1.39 mmol), K₂CO₃ (320.7 mg, 2.32 mmol), Pd(dppf)Cl₂ (84.9 mg, 0.116 mmol) and the reaction stirred at 100° C. for 3 h. The cooled mixture was concentrated in vacuo and the residue purified by column chromatography (SiO₂, 6-50% EtOAc/PE) to afford 4-chloro-2-(1-methyl-1H-pyrazol-4-yl)-1-tosyl-1H-pyrrolo[2,3-b]pyridine as a yellow solid (287 mg, 64%). ¹H NMR (400 MHz, CDCl₃) δ: 8.35-8.30 (m, 1H), 7.75-7.64 (m, 4H), 7.18-7.14 (m, 3H), 6.55 (s, 1H), 4.00 (s, 3H), 2.32 (s, 3H).

### Preparation 52

### 4-chloro-2-(2-methoxypyridin-4-yl)-1-tosyl-1H-pyrrolo[2,3-b]pyridine

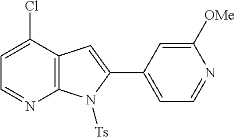 [SMILES: COc1cc(-c2cc3c(Cl)ccnc3n2S(=O)(=O)c2ccc(C)cc2)ccn1]

4-Chloro-2-(2-methoxypyridin-4-yl)-1-tosyl-1H-pyrrolo[2,3-b]pyridine was obtained as a yellow solid (1.6 g, 83%) from 4-chloro-2-iodo-1-tosyl-1H-pyrrolo[2,3-b]pyridine (Preparation 50) and (2-methoxypyridin-4-yl)boronic acid following a similar procedure to that described in Preparation 51. LCMS m/z=414.2 [M+H]⁺.

### Preparation 53

### (3R,4S)-3-cyclopropyl-4-methyl-1-(2-(1-methyl-1H-pyrazol-4-yl)-1-tosyl-1H-pyrrolo[2,3-b]pyridin-4-yl)-2-oxopyrrolidine-3-carbonitrile

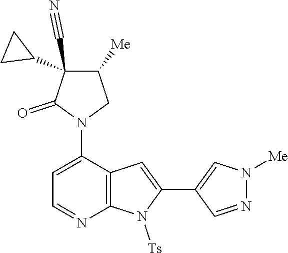 [SMILES: Cc1ccc(S(=O)(=O)n2c(-c3cnn(C)c3)cc3c(N4C[C@@H](C)[C@@](C#N)(C5CC5)C4=O)ccnc32)cc1]

To a solution of 4-chloro-2-(1-methyl-1H-pyrazol-4-yl)-1-tosyl-1H-pyrrolo[2,3-b]pyridine (Preparation 51, 290 mg, 0.75 mmol) and (3R,4S)-3-cyclopropyl-4-methyl-2-oxopyrrolidine-3-carbonitrile (Preparation 21, 307.7 mg, 1.87 mmol) in dioxane (1 mL) was added RuPhos Pd G3 (62.7 mg, 0.075 mmol) and K₂CO₃ (310.8 mg, 2.25 mmol) at 25° C. and the reaction stirred at 100° C. for 2 h under microwave irradiation and N₂. The cooled reaction was concentrated under reduced pressure and purified by column chromatography (6-50% EtOAc/PE) to give (3R,4S)-3-cyclopropyl-4-methyl-1-(2-(1-methyl-1H-pyrazol-4-yl)-1-tosyl-1H-pyrrolo[2,3-b]pyridin-4-yl)-2-oxopyrrolidine-3-carbonitrile as a yellow oil (400 mg, crude) as yellow oil. ¹H NMR (400 MHz, CDCl₃) δ: 8.41 (d, 1H), 7.75-7.73 (m, 3H), 7.67 (s, 1H), 7.19-7.15 (m, 3H), 6.49 (s, 1H), 4.00 (s, 3H), 3.96-3.92 (m, 1H), 3.76-3.71 (m, 1H), 3.55-3.53 (m, 1H), 2.34 (s, 3H), 1.32-1.27 (m, 3H), 1.11-1.10 (m, 1H), 0.78-0.72 (m, 4H).

### Preparation 54

### (S)-3-cyclopropyl-1-(2-(1-methyl-1H-pyrazol-4-yl)-1-tosyl-1H-pyrrolo[2,3-b]pyridin-4-yl)-2-oxopyrrolidine-3-carbonitrile

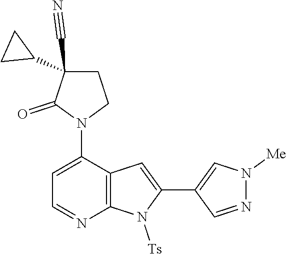 [SMILES: Cc1ccc(S(=O)(=O)n2c(-c3cnn(C)c3)cc3c(N4CC[C@@](C#N)(C5CC5)C4=O)ccnc32)cc1]

To a solution of 4-chloro-2-(1-methyl-1H-pyrazol-4-yl)-1-tosyl-1H-pyrrolo[2,3-b]pyridine (Preparation 51, 350.0 mg, 0.905 mmol) and (S)-3-cyclopropyl-2-oxopyrrolidine-3-carbonitrile (Example 1D, WO2016 0159773, 339.7 mg, 2.26 mmol) in dioxane (10 mL) was added K₂CO₃ (375.1 mg, 2.71 mmol) and RuPhos Pd G3 (75.7 mg, 0.090 mmol) and the reaction stirred at 100° C. for 2 h under microwave irradiation and N₂. The cooled mixture was treated with H₂O (15 mL) and extracted with EtOAc (3×10 mL). The combined organic phase was washed with brine (20 mL), (Na₂SO₄) and concentrated in vacuo. The crude was purified by column chromatography (SiO₂, 25-100% EtOAc/PE) to give (S)-3-cyclopropyl-1-(2-(1-methyl-1H-pyrazol-4-yl)-1-tosyl-1H-pyrrolo[2,3-b]pyridin-4-yl)-2-oxopyrrolidine-3-carbonitrile as a yellow oil (430 mg, 95%). LCMS m/z=501.1 [M+H]⁺

### Preparation 55

### (R)-3-cyclopropyl-1-(2-(1-methyl-1H-pyrazol-4-yl)-1-tosyl-1H-pyrrolo[2,3-b]pyridin-4-yl)-2-oxopyrrolidine-3-carbonitrile

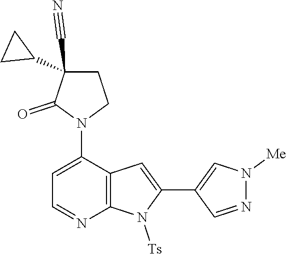 [SMILES: Cc1ccc(S(=O)(=O)n2c(-c3cnn(C)c3)cc3c(N4CC[C@@](C#N)(C5CC5)C4=O)ccnc32)cc1]

(R)-3-cyclopropyl-1-(2-(1-methyl-1H-pyrazol-4-yl)-1-tosyl-1H-pyrrolo[2,3-b]pyridin-4-yl)-2-oxopyrrolidine-3-carbonitrile was prepared as a yellow oil, from 4-chloro-2-(1-methyl-1H-pyrazol-4-yl)-1-tosyl-1H-pyrrolo[2,3-b]pyridine (Preparation 51) and (R)-3-cyclopropyl-2-oxopyrrolidine-3-carbonitrile (WO2016 0159773), following the procedure described in Preparation 54. LCMS m/z=501.1 [M+H]⁺.

### Preparation 56

### (S)-3-cyclopropyl-1-(2-(2-methoxypyridin-4-yl)-1-tosyl-1H-pyrrolo[2,3-b]pyridin-4-yl)-2-oxopyrrolidine-3-carbonitrile

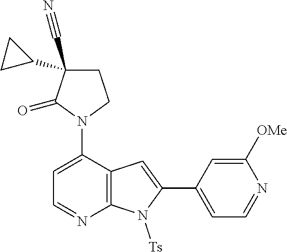 [SMILES: COc1cc(-c2cc3c(N4CC[C@@](C#N)(C5CC5)C4=O)ccnc3n2S(=O)(=O)c2ccc(C)cc2)ccn1]

(S)-3-Cyclopropyl-1-(2-(2-methoxypyridin-4-yl)-1-tosyl-1H-pyrrolo[2,3-b]pyridin-4-yl)-2-oxopyrrolidine-3-carbonitrile was prepared as a yellow solid (80 mg, 70.6%) from 4-chloro-2-(2-methoxypyridin-4-yl)-1-tosyl-1H-pyrrolo[2,3-b]pyridine (Preparation 52) and (S)-3-cyclopropyl-2-oxopyrrolidine-3-carbonitrile (Example 1D, WO2016 0159773), following the procedure described in Preparation 54. LCMS m/z=528.3 [M+H]⁺

### Preparation 57

### (R)-3-cyclopropyl-1-(2-(2-methoxypyridin-4-yl)-1-tosyl-1H-pyrrolo[2,3-b]pyridin-4-yl)-2-oxopyrrolidine-3-carbonitrile

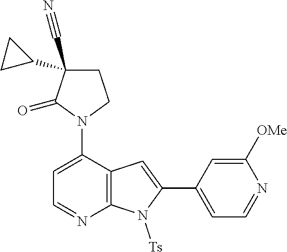 [SMILES: COc1cc(-c2cc3c(N4CC[C@@](C#N)(C5CC5)C4=O)ccnc3n2S(=O)(=O)c2ccc(C)cc2)ccn1]

(R)-3-Cyclopropyl-1-(2-(2-methoxypyridin-4-yl)-1-tosyl-1H-pyrrolo[2,3-b]pyridin-4-yl)-2-oxopyrrolidine-3-carbonitrile was prepared as a white solid, 110 mg, 43.2% yield, from 4-chloro-2-(2-methoxypyridin-4-yl)-1-tosyl-1H-pyrrolo[2,3-b]pyridine (Preparation 52) and (R)-3-cyclopropyl-2-oxopyrrolidine-3-carbonitrile (WO2016 0159773), following the procedure described in Preparation 54. LCMS m/z=528.3 [M+H]⁺

### Preparation 58

### 4-chloro-7-tosyl-7H-pyrrolo[2,3-d]pyrimidine

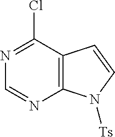 [SMILES: Cc1ccc(S(=O)(=O)n2ccc3c(Cl)ncnc32)cc1]

To a solution of 4-chloro-7H-pyrrolo[2,3-d]pyrimidine (25.0 g, 13.02 mmol) in acetone (250 mL) was added TsCl (37.2 g, 195.4 mmol) and 2M NaOH (97.6 mL) at 0° C. and the reaction stirred at 25° C. for 3 h. The solid was filtered, washed with acetone/H₂O (v/v=1/1), collected and dried under vacuum to afford 4-chloro-7-tosyl-7H-pyrrolo[2,3-d]pyrimidine (45.6 g, 91%) as a white solid. ¹H NMR (400 MHz, CDCl₃) δ: 8.76 (s, 1H), 8.08 (d, 2H), 7.77 (d, 1H), 4.32 (d, 2H), 6.70 (d, 1H), 2.40 (s, 3H).

### Preparation 59

### 4-chloro-6-iodo-7-tosyl-7H-pyrrolo[2,3-d]pyrimidine

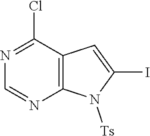 [SMILES: Cc1ccc(S(=O)(=O)n2c(I)cc3c(Cl)ncnc32)cc1]

To a solution of 4-chloro-7-tosyl-7H-pyrrolo[2,3-d]pyrimidine (Preparation 58, 10 g, 32.5 mmol) in THE (200 mL) was added LDA (2 M, 24.37 mL) drop wise at −78° C. under N₂ and the mixture stirred at −78° C. for 1 h. A solution of I₂ (10.7 g, 42.2 mmol) in THE (50 mL) was added drop wise and the mixture stirred at −78° C. for 1 h. The reaction was quenched using sat. aq. NH₄Cl (5 mL), diluted with H₂O (10 mL) and extracted with EtOAc (3×20 mL). The combined organic extracts were washed with brine (10 mL), dried over Na₂SO₄, filtered, and the filtrate concentrated in vacuo. The crude was purified by column chromatography (SiO₂, 9-25% EtOAc/PE) to afford 4-chloro-6-iodo-7-tosyl-7H-pyrrolo[2,3-d]pyrimidine (6.0 g, 42.6%) as a yellow solid. LCMS m/z=433.9 [M+H]⁺

### Preparation 60

### 4-chloro-6-(1-methyl-1H-pyrazol-4-yl)-7-tosyl-7H-pyrrolo[2,3-d]pyrimidine

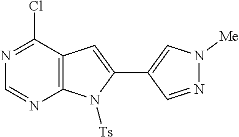 [SMILES: Cc1ccc(S(=O)(=O)n2c(-c3cnn(C)c3)cc3c(Cl)ncnc32)cc1]

To a solution of 4-chloro-6-iodo-7-tosyl-7H-pyrrolo[2,3-d]pyrimidine (Preparation 59, 300 mg, 0.692 mmol) and 1-methyl-4-(4,4,5,5-tetramethyl-1,3,2-dioxaborolan-2-yl)-1H-pyrazole (216 mg, 1.04 mmol) in dioxane (3 mL) and water (0.3 mL) was added Pd(dppf)Cl₂ (50.6 mg, 0.069 mmol) and K₃PO₄ (294 mg, 1.38 mmol) and the reaction stirred at 30° C. for 12 h. The reaction was treated with H₂O (5 mL) and extracted with EtOAc (3×5 mL). The combined organic extracts were washed with brine (10 mL), dried over Na₂SO₄, filtered, concentrated, then purified by prep-TLC (50% PE/EtOAc) to give 4-chloro-6-(1-methyl-1H-pyrazol-4-yl)-7-tosyl-7H-pyrrolo[2,3-d]pyrimidine (129 mg, 48% yield) as rose pink solid. LCMS m/z=388.0 [M+H]⁺

### Preparation 61

### (3R,4S)-3-cyclopropyl-4-methyl-1-(6-(1-methyl-1H-pyrazol-4-yl)-7-tosyl-7H-pyrrolo[2,3-d]pyrimidin-4-yl)-2-oxopyrrolidine-3-carbonitrile

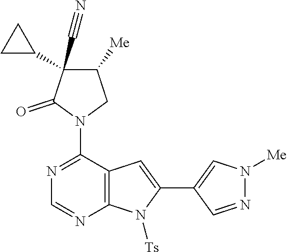 [SMILES: Cc1ccc(S(=O)(=O)n2c(-c3cnn(C)c3)cc3c(N4C[C@@H](C)[C@@](C#N)(C5CC5)C4=O)ncnc32)cc1]

To a solution of 4-chloro-6-(1-methyl-1H-pyrazol-4-yl)-7-tosyl-7H-pyrrolo[2,3-d]pyrimidine (Preparation 60, 100 mg, 0.258 mmol) and (3R,4S)-3-cyclopropyl-4-methyl-2-oxopyrrolidine-3-carbonitrile (Preparation 21, 84.7 mg, 0.516 mmol) in dioxane (5 mL) was added Pd₂(dba)₃ (23.6 mg, 0.0258 mmol), Xantphos (14.9 mg, 0.0258 mmol) and K₃PO₄ (76.6 mg, 0.361 mmol) and the reaction stirred at 70° C. for 12 h under N₂. The mixture was concentrated under reduced pressure and the residue was purified by column chromatography (SiO₂, 6-50% EtOAc/PE) to give (3R,4S)-3-cyclopropyl-4-methyl-1-(6-(1-methyl-1H-pyrazol-4-yl)-7-tosyl-7H-pyrrolo[2,3-d]pyrimidin-4-yl)-2-oxopyrrolidine-3-carbonitrile (160 mg, crude) as colourless gum. LCMS m/z=516.2 [M+H]⁺

### Preparation 62

### 1-(4-bromo-1H-pyrrol-2-yl)ethan-1-one

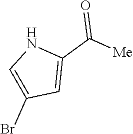

Amberlyst® 15 (3.97 g) was added to a solution of 1-(1H-pyrrol-2-yl)ethan-1-one 1 (44 g, 403 mmol) in THE (442 mL) at rt, the mixture cooled to −30° C. and NBS (71.8 g, 403 mmol) added portion wise. The resulting mixture was stirred for 2 h before warming to rt and filtered. The filtrate was diluted with saturated aq. sodium sulfite and the resulting mixture extracted with DCM (2×). The combined organics were evaporated to dryness in vacuo and the residue dissolved in TBME. The solution was washed with NaHCO₃ (2×), brine, dried (Na₂SO₄) and concentrated under reduced pressure to give 1-(4-bromo-1H-pyrrol-2-yl)ethan-1-one as a white solid which was used without any further purification (76.2 g). LCMS m/z=188.0 [M+H]⁺

### Preparation 63

### (E)-1-(4-bromo-1H-pyrrol-2-yl)-3-(dimethylamino)prop-2-en-1-one

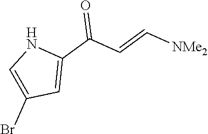 [SMILES: CN(C)/C=C/C(=O)c1cc(Br)c[nH]1]

A solution of 1-(4-bromo-1H-pyrrol-2-yl)ethan-1-one (Preparation 62, 76.2 g, 0.36 mol) in DMF-DMA (390 mL) was heated to 85° C. overnight. The yellow suspension was diluted with heptane and the resulting solid was collected by filtration. The filter cake was washed with heptane and dried to give (E)-1-(4-bromo-1H-pyrrol-2-yl)-3-(dimethylamino)prop-2-en-1-one as a light brown solid (61.5 g, 69%). LCMS m/z=243.1 [M+H]⁺

### Preparation 64

### 6-bromopyrrolo[1,2-b]pyridazin-4-ol

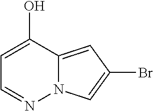 [SMILES: Oc1ccnn2cc(Br)cc12]

KO^{t}Bu (42.6 g, 379 mmol) was added portion wise to a solution of (E)-1-(4-bromo-1H-pyrrol-2-yl)-3-(dimethylamino)prop-2-en-1-one (Preparation 63, 61.5 g, 253 mmol) in NMP (1.85 L) and the mixture stirred at rt for 30 mins. The reaction mixture was cooled on an ice/water bath and O-(4-nitrobenzoyl)hydroxylamine (69.1 g, 379 mmol) was added and the resulting mixture was stirred at 0° C. for 1 h and then overnight at rt. The mixture was cooled to 0° C. and saturated aq. NH₄Cl (500 mL) was added dropwise. The mixture was diluted with H₂O (500 mL) and the pH adjusted to 3-4 with aq. 2 N HCl and extracted with TBME (3×). The combined organics were concentrated to half of the volume and washed with H₂O (2×), brine and then evaporated to dryness in vacuo. The residue was filtered through a pad of silica (0-100% EtOAc/heptane) to afford 6-bromopyrrolo[1,2-b]pyridazin-4-ol (58 g, 54%) which was used without any further purification. LCMS m/z=213.0 [M+H]⁺

### Preparation 65

### 6-bromopyrrolo[1,2-b]pyridazin-4-yl trifluoromethanesulfonate

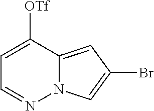 [SMILES: O=S(=O)(Oc1ccnn2cc(Br)cc12)C(F)(F)F]

Trifluoromethanesulfonic anhydride (88.3 g, 313 mmol) was added to a solution of 6-bromopyrrolo[1,2-b]pyridazin-4-ol (Preparation 64, 58 g, ~140 mmol, ~50% pure) and TEA (32.5 g, 321 mmol) in DCM (870 mL) at 0° C. and the mixture stirred for 1 h with cooling and then at rt for 2 h. The mixture was diluted with DCM and washed with Na₂CO₃ (2×), brine, dried (Na₂SO₄) and evaporated to dryness in vacuo. The residue was purified by column chromatography (SiO₂, 0-2% TBME/heptane) to give 6-bromopyrrolo[1,2-b]pyridazin-4-yl trifluoromethanesulfonate as a dark oil (21.1 g, 43%). LCMS m/z=344.6 [M+H]⁺

### Preparation 66

### 1-(6-bromopyrrolo[1,2-b]pyridazin-4-yl)-2-oxopyrrolidine-3-carbonitrile

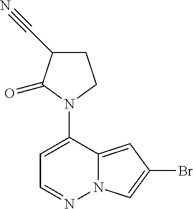 [SMILES: N#CC1CCN(c2ccnn3cc(Br)cc23)C1=O]

To a solution of 6-bromopyrrolo[1,2-b]pyridazin-4-yl trifluoromethanesulfonate (Preparation 65, 100 mg, 0.290 mmol), 2-oxopyrrolidine-3-carbonitrile (47.9 mg, 0.435 mmol), Xantphos (16.8 mg, 0.029 mmol), Pd₂(dba)₃ (26.5 mg, 0.029 mmol) and K₂CO₃ (80.1 mg, 0.580 mmol) in toluene (2 mL) was stirred at 80° C. for 2 h under N₂. The cooled mixture was concentrated under reduced pressure and purified by column chromatography (4-50% EtOAc/PE) to give 1-(6-bromopyrrolo[1,2-b]pyridazin-4-yl)-2-oxopyrrolidine-3-carbonitrile (50 mg, 56.6%) as a yellow solid. LCMS m/z=305.0 [M+H]⁺

### Preparation 67

### 1-(6-bromopyrrolo[1,2-b]pyridazin-4-yl)-3-(methoxymethyl)-2-oxopyrrolidine-3-carbonitrile

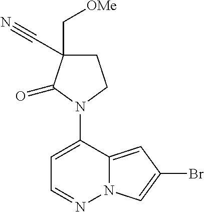 [SMILES: COCC1(C#N)CCN(c2ccnn3cc(Br)cc23)C1=O]

To a solution of 6-bromopyrrolo[1,2-b]pyridazin-4-yl trifluoromethanesulfonate (Preparation 65, 100 mg, 0.290 mmol) and 3-(methoxymethyl)-2-oxopyrrolidine-3-carbonitrile (67 mg, 0.435 mmol) in THE (5 mL) was added Xantphos (16.8 mg, 0.029 mmol), Pd₂(dba)₃ (26.5 mg, 0.029 mmol) and Cs₂CO₃ (132 mg, 0.406 mmol) and the reaction stirred at 70° C. for 4 h under N₂. The cooled reaction was concentrated under reduced pressure and the residue was purified by column chromatography (SiO₂, 6-50% EtOAc/PE) to give 1-(6-bromopyrrolo[1,2-b]pyridazin-4-yl)-3-(methoxymethyl)-2-oxopyrrolidine-3-carbonitrile (95 mg, 94%) as yellow oil. LCMS m/z=349.0 [M+H]⁺

### Preparation 68

### (3R,4S)-1-(6-bromopyrrolo[1,2-b]pyridazin-4-yl)-3-cyclopropyl-4-methyl-2-oxopyrrolidine-3-carbonitrile

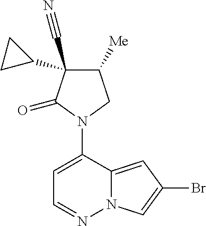 [SMILES: C[C@@H]1CN(c2ccnn3cc(Br)cc23)C(=O)[C@]1(C#N)C1CC1]

To a solution of 6-bromopyrrolo[1,2-b]pyridazin-4-yl trifluoromethanesulfonate (Preparation 65, 2.0 g, 5.80 mmol), (3R,4S)-3-cyclopropyl-4-methyl-2-oxopyrrolidine-3-carbonitrile (Preparation 21, (1.05 g, 6.38 mmol), Pd₂(dba)₃ (530.7 mg, 0.58 mmol), Xantphos (335.3 mg, 0.58 mmol) and K₃PO₄ (1.72 g, 8.11 mmol) in dioxane (20 mL) was stirred at 70° C. for 1 h under N₂. The mixture was concentrated in vacuo and the crude product was purified by column chromatography (SiO₂, 9-25% EtOAc/PE) to give (3R,4S)-1-(6-bromopyrrolo[1,2-b]pyridazin-4-yl)-3-cyclopropyl-4-methyl-2-oxopyrrolidine-3-carbonitrile (3.2 g, crude) as a yellow solid. ¹H NMR (400 MHz, CDCl₃) δ: 8.01 (d, 1H), 7.79 (s, 1H), 6.94 (d, 1H), 6.61 (s, 1H), 4.13-4.06 (m, 1H), 3.80-3.50 (m, 1H), 3.11-3.01 (m, 1H), 1.41 (d, 3H), 1.22-1.13 (m, 1H), 0.80-0.75 (m, 4H).

### Preparation 69

### (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile

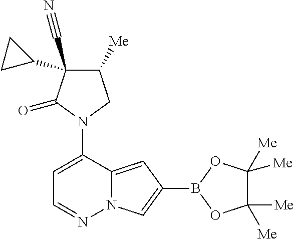 [SMILES: C[C@@H]1CN(c2ccnn3cc(B4OC(C)(C)C(C)(C)O4)cc23)C(=O)[C@]1(C#N)C1CC1]

A mixture of (3R,4S)-1-(6-bromopyrrolo[1,2-b]pyridazin-4-yl)-3-cyclopropyl-4-methyl-2-oxopyrrolidine-3-carbonitrile (Preparation 68, 10.2 g, 28.39 mmol), B₂Pin₂ (14.42 g, 56.79 mmol), AcOK (8.36 g, 85.18 mmol) in dioxane (150 mL) was added Pd(dppf)Cl₂·DCM (2.32 g, 2.84 mmol) under N₂ and the mixture stirred at 90° C. for 16 h. The reaction was filtered and the filtrate evaporated to dryness in vacuo and the residue purified by column chromatography (SiO₂, 25% EtOAc/PE) to give (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile as a yellow solid (6 g, 49.5%). LCMS m/z=407.2 [M+H]⁺

### Preparation 70

### (S)-1-(6-bromopyrrolo[1,2-b]pyridazin-4-yl)-3-cyclopropyl-2-oxopyrrolidine-3-carbonitrile

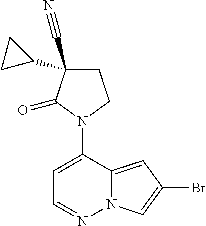 [SMILES: N#C[C@@]1(C2CC2)CCN(c2ccnn3cc(Br)cc23)C1=O]

To a solution of 6-bromopyrrolo[1,2-b]pyridazin-4-yl trifluoromethanesulfonate (Preparation 65, 200.0 mg, 0.58 mmol) and (S)-3-cyclopropyl-2-oxopyrrolidine-3-carbonitrile (Example 1D, WO2016 0159773, 95.7 mg, 0.638 mmol) in dioxane (8 mL) was added Pd₂(dba)₃ (53.1 mg, 0.058 mmol), Xantphos (33.5 mg, 0.058 mmol) and K₃PO₄ (172.2 mg, 0.812 mmol) and the reaction stirred at 70° C. for 2 h under N₂. The reaction was washed with H₂O (10 mL) and extracted with EtOAc (3×10 mL). The combined organic extracts were washed with brine (15 mL), dried (Na₂SO₄), evaporated to dryness and purified by column chromatography (SiO₂, 6-50% EtOAc/PE) to give (S)-1-(6-bromopyrrolo[1,2-b]pyridazin-4-yl)-3-cyclopropyl-2-oxopyrrolidine-3-carbonitrile (195 mg, 97.5%) as yellow solid. LCMS m/z=347.1 [M+H]⁺

### Preparations 71 to 93

The following compounds were prepared from 6-bromopyrrolo[1,2-b]pyridazin-4-yl trifluoromethanesulfonate (Preparation 65) and the appropriate lactam following a similar procedure to that described in Preparation 70.

| Preparation | |
|---|---|
| No | Name/Structure/Lactam/Data |
| 71 | 1-(6-bromopyrrolo[1,2-b]pyridazin-4-yl)-3-cyclopropylpyrrolidin-2-one |
| | 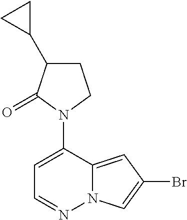 [SMILES: O=C1C(C2CC2)CCN1c1ccnn2cc(Br)cc12] |
| | Lactam: 3-cyclopropylpyrrolidine-2-one |
| | 245 mg, 88% yield as a yellow solid. LCMS m/z = 322.0 [M + H]⁺ |
| 72 | 1-(6-bromopyrrolo[1,2-b]pyridazin-4-yl)-3-methyl-2-oxopyrrolidine-3- |
| | carbonitrile |
| | 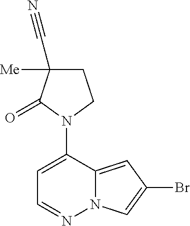 [SMILES: CC1(C#N)CCN(c2ccnn3cc(Br)cc23)C1=O] |
| | Lactam: 3-methyl-2-oxopyrrolidine-3-carbonitrile |
| | 110 mg, 79.3% yield as yellow gum. LCMS m/z = 319.1 [M + H]⁺ |
| 73 | 1-(6-bromopyrrolo[1,2-b]pyridazin-4-yl)-3-ethyl-2-oxopyrrolidine-3- |
| | carbonitrile |
| | 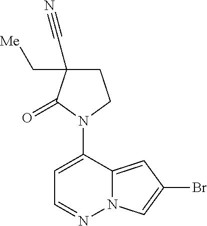 [SMILES: CCC1(C#N)CCN(c2ccnn3cc(Br)cc23)C1=O] |
| | Lactam: 3-ethyl-2-oxopyrrolidine-3-carbonitrile (Preparation 1) |
| | 230 mg, 79.4% yellow oil |
| | ¹H NMR (500 MHz, CDCl₃) δ: 8.02 (d, 1H), 7.80 (s, 1H), 6.85 (d, 1H), 6.60 |
| | (s, 1H), 4.14-4.11 (m, 1H), 4.07-4.03 (m, 1H), 2.74-2.72 (m, 1H), 2.35-2.21 |
| | (m, 2H), 1.91-1.86 (m, 1H), 1.26-1.24 (m, 3H). |
| 74 | 1-(6-bromopyrrolo[1,2-b]pyridazin-4-yl)-3-cyclobutyl-2-oxopyrrolidine-3- |
| | carbonitrile |
| | 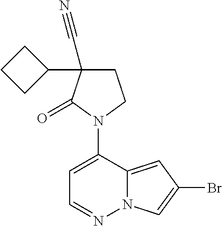 [SMILES: N#CC1(C2CCC2)CCN(c2ccnn3cc(Br)cc23)C1=O] |
| | Lactam: 3-cyclobutyl-2-oxopyrrolidine-3-carbonitrile (Preparation 2) |
| | 260 mg, 83.3% yield as a yellow oil |
| | ¹H NMR (500 MHz, CDCl₃) δ: 8.01 (d, 1H), 7.80 (s, 1H), 6.84 (d, 1H), 6.59 |
| | (s, 1H), 4.10-4.02 (m, 2H), 2.90-2.87 (m, 1H), 2.68-2.67 (m, 1H), 2.34-2.17 |
| | (m, 5H), 2.00-1.98 (m, 2H). |
| 75 | 1-(6-bromopyrrolo[1,2-b]pyridazin-4-yl)-3-(oxetan-3-yl)-2-oxopyrrolidine-3- |
| | carbonitrile |
| | 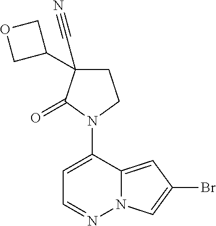 [SMILES: N#CC1(C2COC2)CCN(c2ccnn3cc(Br)cc23)C1=O] |
| | Lactam: 3-(oxetan-3-yl)-2-oxopyrrolidine-3-carbonitrile (Preparation 12) |
| | 290 mg, 96.4% yield as yellow oil. LCMS m/z = 361.0 [M + H]⁺ |
| 76 | (R)-1-(6-bromopyrrolo[1,2-b]pyridazin-4-yl)-3-cyclopropyl-2- |
| | oxopyrrolidine-3-carbonitrile |
| | 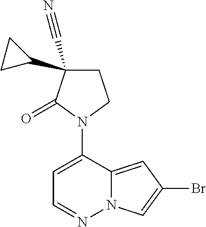 [SMILES: N#C[C@]1(C2CC2)CCN(c2ccnn3cc(Br)cc23)C1=O] |
| | Lactam: (R)-3-cyclopropyl-2-oxopyrrolidine-3-carbonitrile (WO2016 |
| | 0159773) |
| | 184.2 mg, 92.1% yield as a light yellow solid |
| | ¹H NMR (500 MHz, CDCl₃) δ: 8.03 (d, 1H), 7.81 (d, 1H), 6.88 (d, 1H), 6.62 |
| | (d, 1H), 4.14-4.10 (m, 2H), 2.78-2.73 (m, 1H), 2.49-2.45 (m, 1H), 1.37-1.34 |
| | (m, 1H), 0.86-0.75 (m, 4H). |
| 77 | (R)-1-(6-bromopyrrolo[1,2-b]pyridazin-4-yl)-3-cyclopropyl-3- |
| | (difluoromethyl)pyrrolidin-2-one |
| | 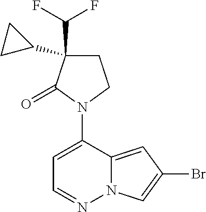 [SMILES: O=C1N(c2ccnn3cc(Br)cc23)CC[C@@]1(C(F)F)C1CC1] |
| | Lactam: (R)-3-cyclopropyl-3-(difluoromethyl)pyrrolidin-2-one (Preparation |
| | 44) |
| | 93.2 mg, 86.9% yield as yellow oil. LCMS m/z = 372.0 [M + H]⁺ |
| 78 | (3R,5R)-1-(6-bromopyrrolo[1,2-b]pyridazin-4-yl)-3-cyclopropyl-5-methyl-2- |
| | oxopyrrolidine-3-carbonitrile |
| | 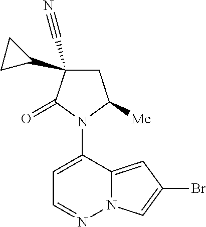 [SMILES: C[C@@H]1C[C@](C#N)(C2CC2)C(=O)N1c1ccnn2cc(Br)cc12] |
| | Lactam: (3R,5R)-3-cyclopropyl-5-methyl-2-oxopyrrolidine-3-carbonitrile |
| | (Preparation 15) |
| | 100 mg, 96.1% yield as yellow oil |
| | ¹H NMR (500 MHz, CDCl₃) δ: 8.06 (d, 1H), 7.82 (s, 1H), 6.55-6.51 (m, 2H), |
| | 4.53-4.46 (m, 1H), 2.90-2.86 (m, 1H), 1.96-1.91 (m, 1H), 1.37-1.24 (m, 1H), |
| | 1.25-1.23 (m, 3H), 0.89-0.73 (m, 4H). |
| 79 | (3S,5S)-1-(6-bromopyrrolo[1,2-b]pyridazin-4-yl)-3-cyclopropyl-5-methyl-2- |
| | oxopyrrolidine-3-carbonitrile |
| | 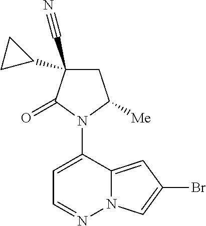 [SMILES: C[C@H]1C[C@@](C#N)(C2CC2)C(=O)N1c1ccnn2cc(Br)cc12] |
| | Lactam: (3S,5S)-3-cyclopropyl-5-methyl-2-oxopyrrolidine-3-carbonitrile |
| | (Preparation 18) |
| | 50 mg, 48% yield as light green solid |
| | ¹H NMR (500 MHz, CDCl₃) δ: LCMS m/z = 359.0 [M + H]⁺ |
| 80 | (3S,5R)-1-(6-bromopyrrolo[1,2-b]pyridazin-4-yl)-3-cyclopropyl-5-methyl-2- |
| | oxopyrrolidine-3-carbonitrile |
| | 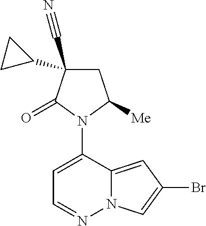 [SMILES: C[C@@H]1C[C@@](C#N)(C2CC2)C(=O)N1c1ccnn2cc(Br)cc12] |
| | Lactam: (3S,5R)-3-cyclopropyl-5-methyl-2-oxopyrrolidine-3-carbonitrile |
| | (Preparation 14) |
| | 96 mg, 92.1% yield as yellow oil. LCMS m/z = 359.0 [M + H]⁺ |
| 81 | (3S,5R)-1-(6-bromopyrrolo[1,2-b]pyridazin-4-yl)-3-cyclopropyl-5-ethyl-2- |
| | oxopyrrolidine-3-carbonitrile |
| | 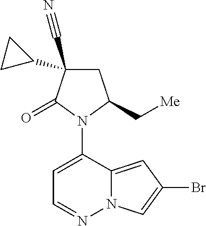 [SMILES: CC[C@@H]1C[C@@](C#N)(C2CC2)C(=O)N1c1ccnn2cc(Br)cc12] |
| | Lactam: (3S,5R)-3-cyclopropyl-5-ethyl-2-oxopyrrolidine-3-carbonitrile |
| | (Preparation 30) |
| | 90.0 mg, 69.3% yield as a yellow oil. |
| | ¹H NMR (500 MHz, CDCl₃) δ: 8.07 (d, 1H), 7.81 (s, 1H), 6.62 (d, 1H), 6.50 |
| | (s, 1H), 4.34-4.29 (m, 1H), 2.65-2.61 (m, 1H), 2.46-2.43 (m, 1H), 1.71-1.68 |
| | (m, 1H), 1.51-1.49 (m, 1H), 1.35-1.34 (m, 1H), 0.90-0.86 (m, 3H), 0.78-0.72 |
| | (m, 4H). |
| 82 | (3S,5S)-1-(6-bromopyrrolo[1,2-b]pyridazin-4-yl)-3-cyclopropyl-5-ethyl-2- |
| | oxopyrrolidine-3-carbonitrile |
| | 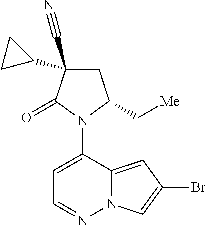 [SMILES: CC[C@H]1C[C@@](C#N)(C2CC2)C(=O)N1c1ccnn2cc(Br)cc12] |
| | Lactam: (3S,5S)-3-cyclopropyl-5-ethyl-2-oxopyrrolidine-3-carbonitrile |
| | (Preparation 27) |
| | 90.0 mg, 69.3% yield as a yellow oil. |
| 83 | (3S,5S)-1-(6-bromopyrrolo[1,2-b]pyridazin-4-yl)-4-methyl-3-(oxetan-3-yl)- |
| | 2-oxopyrrolidine-3-carbonitrile |
| | 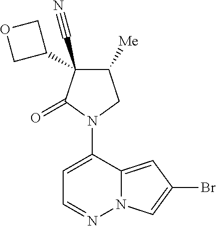 [SMILES: C[C@@H]1CN(c2ccnn3cc(Br)cc23)C(=O)[C@]1(C#N)C1COC1] |
| | Lactam: (3S,4S)-4-methyl-3-(oxetan-3-yl)-2-oxopyrrolidine-3-carbonitrile |
| | (Preparation 23) |
| | 100 mg, 92.0% yield, yellow gum. LCMS m/z = 375.0 [M + H]⁺ |
| 84 | (3S,4S)-1-(6-bromopyrrolo[1,2-b]pyridazin-4-yl)-3-cyclopropyl-4-methyl-2- |
| | oxopyrrolidine-3-carbonitrile |
| | 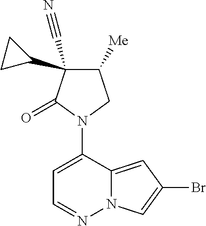 [SMILES: C[C@@H]1CN(c2ccnn3cc(Br)cc23)C(=O)[C@@]1(C#N)C1CC1] |
| | Lactam: (3S,4S)-3-cyclopropyl-4-methyl-2-oxopyrrolidine-3-carbonitrile |
| | (Preparation 22) |
| | 90.0 mg, 86.5% yield as a yellow oil. |
| | ¹H NMR (500 MHz, CDCl₃) δ: 8.01 (d, 1H), 7.79 (s, 1H), 6.87 (d, 1H), 6.61 |
| | (d, 1H), 4.15-4.09 (m, 1H), 3.81-3.73 (m, 1H), 2.71-2.63 (m, 1H), 1.44 (d, |
| | 3H), 1.44-1.18 (m, 1H), 0.87-0.80 (m, 4H). |
| 85 | (3R,4R)-1-(6-bromopyrrolo[1,2-b]pyridazin-4-yl)-3-cyclopropyl-4-methyl-2- |
| | oxopyrrolidine-3-carbonitrile |
| | 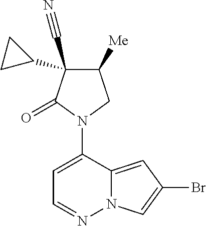 [SMILES: C[C@H]1CN(c2ccnn3cc(Br)cc23)C(=O)[C@]1(C#N)C1CC1] |
| | Lactam: (3R,4R)-3-cyclopropyl-4-methyl-2-oxopyrrolidine-3-carbonitrile |
| | (Preparation 20) |
| | 82 mg, 78.8% yield as a yellow solid. LCMS m/z = 359.1 [M + H]⁺ |
| 86 | (3R,4S)-1-(6-bromopyrrolo[1,2-b]pyridazin-4-yl)-3-cyclopropyl-4-ethyl-2- |
| | oxopyrrolidine-3-carbonitrile |
| | 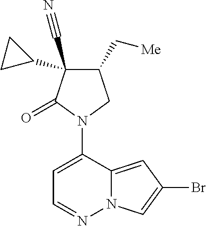 [SMILES: CC[C@@H]1CN(c2ccnn3cc(Br)cc23)C(=O)[C@]1(C#N)C1CC1] |
| | Lactam: (3R,4S)-3-cyclopropyl-4-ethyl-2-oxopyrrolidine-3-carbonitrile |
| | (Preparation 39), yellow oil, 105 mg, 97% yield LCMS m/z = 373.0 |
| | [M + H]⁺ |
| 87 | (3R,4R)-1-(6-bromopyrrolo[1,2-b]pyridazin-4-yl)-3-cyclopropyl-4-ethyl-2- |
| | oxopyrrolidine-3-carbonitrile |
| | 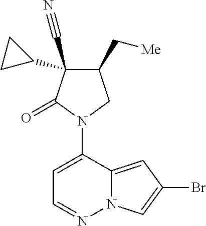 [SMILES: CC[C@H]1CN(c2ccnn3cc(Br)cc23)C(=O)[C@]1(C#N)C1CC1] |
| | Lactam: (3R,4R)-3-cyclopropyl-4-ethyl-2-oxopyrrolidine-3-carbonitrile |
| | (Preparation 35), 105.8 mg, 97.8% yield as yellow oil LCMS m/z = 373.0 |
| | [M + H]⁺ |
| 88 | (R)-2-(6-bromopyrrolo[1,2-b]pyridazin-4-yl)-3-cyclopropyl-2- |
| | oxopyrrolidin-3-yl)acetonitrile |
| | 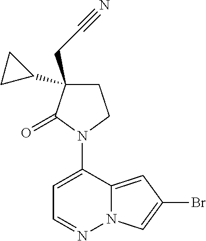 [SMILES: N#CC[C@@]1(C2CC2)CCN(c2ccnn3cc(Br)cc23)C1=O] |
| | Lactam: (R)-2-(3-cyclopropyl-2-oxopyrrolidin-3-yl)acetonitrile |
| | (Preparation 48) |
| | 96.6 mg, 84.4% yield as yellow oil. LCMS m/z = 361.0 [M + H]⁺ |
| 89 | (3R,5S)-1-(6-bromopyrrolo[1,2-b]pyridazin-4-yl)-3-cyclopropyl-5-methyl-2- |
| | oxopyrrolidine-3-carbonitrile |
| | 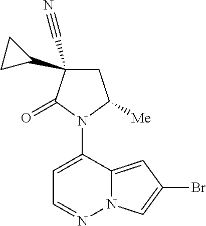 [SMILES: C[C@H]1C[C@](C#N)(C2CC2)C(=O)N1c1ccnn2cc(Br)cc12] |
| | Lactam: (3R,5S)-3-cyclopropyl-5-methyl-2-oxopyrrolidine-3-carbonitrile |
| | (Preparation 17), 90 mg, 86% yield as a yellow oil. ¹H NMR (400 MHz, |
| | CDCl₃) δ: 8.07 (d, 1H), 7.82-7.78 (m, 1H), 6.64 (d, 1H), 6.51 (d, 1H), 4.53- |
| | 4.44 (m, 1H), 2.73-2.67 (m, 1H), 2.42-2.36 (m, 1H), 1.36-1.32 (m, 1H), 1.26- |
| | 1.24 (m, 3H), 0.80-0.72 (m, 4H). |
| 90 | (3R,5R)-1-(6-bromopyrrolo[1,2-b]pyridazin-4-yl)-3-cyclopropyl-5-ethyl-2- |
| | oxopyrrolidine-3-carbonitrile |
| | 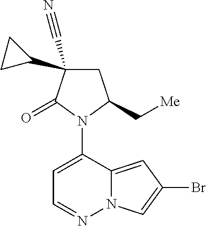 [SMILES: CC[C@@H]1C[C@](C#N)(C2CC2)C(=O)N1c1ccnn2cc(Br)cc12] |
| | Lactam: (3R,5R)-3-cyclopropyl-5-ethyl-2-oxopyrrolidine-3-carbonitrile |
| | (Preparation 31), 60 mg, 48% yield as a green oil. LCMS m/z = 375.0 |
| | [M + H]⁺ |
| 91 | (3R,5S)-1-(6-bromopyrrolo[1,2-b]pyridazin-4-yl)-3-cyclopropyl-5-ethyl-2- |
| | oxopyrrolidine-3-carbonitrile |
| | 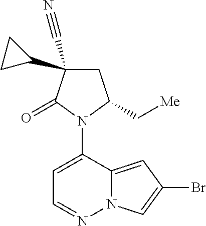 [SMILES: CC[C@H]1C[C@](C#N)(C2CC2)C(=O)N1c1ccnn2cc(Br)cc12] |
| | Lactam: (3R,5S)-3-cyclopropyl-5-ethyl-2-oxopyrrolidine-3-carbonitrile |
| | (Preparation 26), 120 mg as a yellow solid. LCMS m/z = 373.0 [M + H]⁺ |
| 92 | (3S,4R)-1-(6-bromopyrrolo[1,2-b]pyridazin-4-yl)-3-cyclopropyl-4-ethyl-2- |
| | oxopyrrolidine-3-carbonitrile |
| | 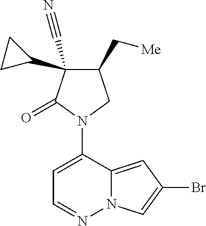 [SMILES: CC[C@H]1CN(c2ccnn3cc(Br)cc23)C(=O)[C@@]1(C#N)C1CC1] |
| | Lactam: (3S,4R)-3-cyclopropyl-4-ethyl-2-oxopyrrolidine-3-carbonitrile |
| | (Preparation 34), 120 mg as a yellow solid. LCMS m/z = 373.0 [M + H]⁺ |
| 93 | (3S,4S)-1-(6-bromopyrrolo[1,2-b]pyridazin-4-yl)-3-cyclopropyl-4-ethyl-2- |
| | oxopyrrolidine-3-carbonitrile |
| | 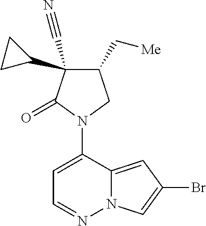 [SMILES: CC[C@@H]1CN(c2ccnn3cc(Br)cc23)C(=O)[C@@]1(C#N)C1CC1] |
| | Lactam: (3S,4S)-3-cyclopropyl-4-ethyl-2-oxopyrrolidine-3-carbonitrile |
| | (Preparation 38), 120 mg as a yellow solid. LCMS m/z = 373.0 [M + H]⁺ |

### Preparation 94

### (3R,4S)-1-(6-bromo-7-fluoropyrrolo[1,2-b]pyridazin-4-yl)-3-cyclopropyl-4-methyl-2-oxopyrrolidine-3-carbonitrile

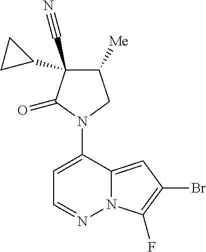 [SMILES: C[C@@H]1CN(c2ccnn3c(F)c(Br)cc23)C(=O)[C@]1(C#N)C1CC1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 100 mg, 0.278 mmol) in MeCN (2.8 mL) was added Select-F (118.3 mg, 0.334 mmol) at 0° C. and the reaction stirred at this temperature for 30 min. The mixture was concentrated in vacuo and purified by column chromatography (25% EtOAc/PE) to give (3R,4S)-1-(6-bromo-7-fluoropyrrolo[1,2-b]pyridazin-4-yl)-3-cyclopropyl-4-methyl-2-oxopyrrolidine-3-carbonitrile (40 mg, 38.1%) as a yellow solid. LCMS m/z=377.0 [M+H]⁺

### Preparation 95

### 2-bromo-8-hydroxyimidazo[1,2-b]pyridazine

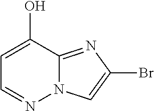 [SMILES: Oc1ccnn2cc(Br)nc12]

A mixture of 2-bromo-8-hydroxyimidazo[1,2-b]pyridazine-7-carboxylic acid (Intermediate 1, step 5, WO2014/039595, 300 mg, 1.16 mmol) in conc. HCl (5 mL, 36% purity) was stirred at 100° C. for 15 h. The cooled mixture was evaporated under reduced pressure and the residue purified by HPLC-1 (gradient 17-47%) to give 2-bromo-8-hydroxyimidazo[1,2-b]pyridazine (60 mg, 24.1%) as white solid. LCMS m/z=214.2, 216.2 [M+H]⁺

### Preparation 96

### 2-bromo-8-chloroimidazo[1,2-b]pyridazine

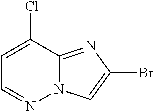 [SMILES: Clc1ccnn2cc(Br)nc12]

2-Bromo-8-hydroxyimidazo[1,2-b]pyridazine (Preparation 95, 20.0 mg, 0.093 mmol) and DIPEA (36.2 mg, 0.28 mmol) were added to POCl₃ (3.29 g, 21.46 mmol) slowly and the reaction stirred at 120° C. for 3 h. Further POCl₃ (10.0 mL) was added and the reaction stirred at 120° C. for an additional 15 h. The cooled reaction was concentrated in vacuo, quenched with H₂O (1.0 mL) and then purified by prep-HPLC-1 (gradient 25-55%) to give 2-bromo-8-chloroimidazo[1,2-b]pyridazine (21 mg, 97%) as yellow solid. LCMS m/z=234.0 [M+H]⁺

### Preparation 97

### (3R,4S)-1-(2-bromoimidazo[1,2-b]pyridazin-8-yl)-3-cyclopropyl-4-methyl-2-oxopyrrolidine-3-carbonitrile

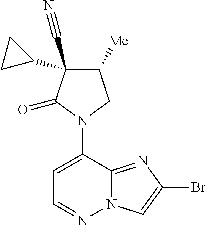 [SMILES: C[C@@H]1CN(c2ccnn3cc(Br)nc23)C(=O)[C@]1(C#N)C1CC1]

To a solution of 2-bromo-8-chloroimidazo[1,2-b]pyridazine (Preparation 96, 100 mg, 0.430 mmol), (3R,4S)-3-cyclopropyl-4-methyl-2-oxopyrrolidine-3-carbonitrile (Preparation 21, 77.7 mg, 0.473 mmol) in dioxane (2 mL) was added Pd₂(dba)₃ (39.4 mg, 0.043 mmol), Xantphos (24.9 mg, 0.043 mmol), K₃PO₄ (127.9 mg, 0.602 mmol) and the mixture stirred at 70° C. for 3 h under N₂. The cooled mixture was concentrated in vacuo and the crude was purified column chromatography (SiO₂, 0-10% EtOAc/PE) to give (3R,4S)-1-(2-bromoimidazo[1,2-b]pyridazin-8-yl)-3-cyclopropyl-4-methyl-2-oxopyrrolidine-3-carbonitrile (100 mg, 64.5%) as a green oil. LCMS m/z=360.0 [M+H]⁺; ¹H NMR (400 MHz, CDCl₃) δ: 8.29-8.26 (m, 1H), 7.97-7.90 (m, 2H), 4.80-4.74 (m, 1H), 4.37-4.31 (m, 1H), 3.01-2.98 (m, 1H), 1.43 (d, 3H), 1.20-1.17 (m, 1H), 0.80-0.70 (m, 4H).

### Preparation 98

### 6-bromo-4-chloropyrrolo[2,1-f][1,2,4]triazine

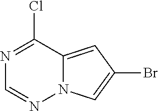 [SMILES: Clc1ncnn2cc(Br)cc12]

A solution of 6-bromopyrrolo[2,1-f][1,2,4]triazin-4(3H)-one (3.0 g, 14.0 mmol) in POCl₃ (50 mL) was stirred at 100° C. for 3 h. The reaction mixture was evaporated in vacuo, the residue dissolved in DCM (60 mL) and the pH adjusted to ~7 with aq. NaHCO₃. The combined organics were dried (Na₂SO₄) and evaporated to dryness in vacuo. The residue was purified by column chromatography (SiO₂, 20-35% EtOAc/PE) to give 6-bromo-4-chloropyrrolo[2,1-f][1,2,4]triazine as a white solid (2.6 g, 80%). LCMS m/z=231.5 [M+H]⁺.

### Preparation 99

### (3R,4S)-1-(6-bromopyrrolo[2,1-f][1,2,4]triazin-4-yl)-3-cyclopropyl-4-methyl-2-oxopyrrolidine-3-carbonitrile

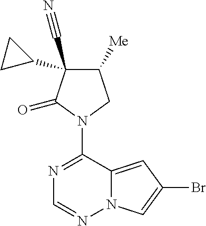 [SMILES: C[C@@H]1CN(c2ncnn3cc(Br)cc23)C(=O)[C@]1(C#N)C1CC1]

(3R,4S)-1-(6-bromopyrrolo[2,1-f][1,2,4]triazin-4-yl)-3-cyclopropyl-4-methyl-2-oxopyrrolidine-3-carbonitrile was obtained as a white solid, 50.0 mg, 32.3% yield, from 6-bromo-4-chloropyrrolo[2,1-f][1,2,4]triazine and (3R,4S)-3-cyclopropyl-4-methyl-2-oxopyrrolidine-3-carbonitrile (Preparation 21) following the procedure described in Preparation 97. LCMS m/z=360.0 [M+H]⁺

### Preparation 100

### (3R,4S)-1-(2-amino-3-nitropyridin-4-yl)-3-cyclopropyl-4-methyl-2-oxopyrrolidine-3-carbonitrile

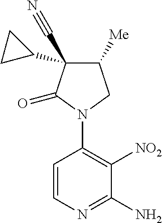 [SMILES: C[C@@H]1CN(c2ccnc(N)c2[N+](=O)[O-])C(=O)[C@]1(C#N)C1CC1]

To a solution of (3R,4S)-3-cyclopropyl-4-methyl-2-oxopyrrolidine-3-carbonitrile (Preparation 21, 94.6 mg, 0.576 mmol) in DMSO (5.0 mL) was added NaH (25.4 mg, 0.634 mmol, 60% purity) and the mixture stirred for 1 h. 4-Chloro-3-nitropyridin-2-amine (100 mg, 0.576 mmol) in DMSO (1.0 mL) was added and the reaction stirred at 20° C. for 2 h. The mixture was quenched with water (0.5 mL), then purified by prep-HPLC-2 (gradient 35-65%) to give (3R,4S)-1-(2-amino-3-nitropyridin-4-yl)-3-cyclopropyl-4-methyl-2-oxopyrrolidine-3-carbonitrile (25 mg, 14.4%) as a yellow solid. LCMS m/z=302.2 [M+H]⁺

### Preparation 101

### (3R,4S)-3-cyclopropyl-1-(2,3-diaminopyridin-4-yl)-4-methyl-2-oxopyrrolidine-3-carbonitrile

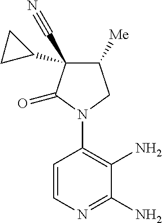 [SMILES: C[C@@H]1CN(c2ccnc(N)c2N)C(=O)[C@]1(C#N)C1CC1]

To a solution of (3R,4S)-1-(2-amino-3-nitropyridin-4-yl)-3-cyclopropyl-4-methyl-2-oxopyrrolidine-3-carbonitrile (Preparation 100, 30 mg, 0.10 mmol) in MeOH (10.0 mL) was added Pd/C (6 mg, 10% purity) and the reaction stirred at 20° C. for 30 mins under 15 Psi of H₂. The mixture was filtered and the filter cake was washed with MeOH. The filtrate was concentrated in vacuo to give (3R,4S)-3-cyclopropyl-1-(2,3-diaminopyridin-4-yl)-4-methyl-2-oxopyrrolidine-3-carbonitrile (30 mg, crude) as yellow oil. LCMS m/z=272.1 [M+H]⁺

### Preparation 102

### Dimethyl 1-(2-(1-methyl-1H-pyrazol-4-yl)-2-oxoethyl)-1H-pyrazole-3,5-dicarboxylate

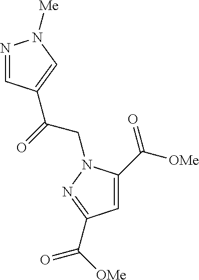 [SMILES: COC(=O)c1cc(C(=O)OC)n(CC(=O)c2cnn(C)c2)n1]

To a mixture of 2-bromo-1-(1-methyl-1H-pyrazol-4-yl)ethan-1-one (100 g, 543.04 mmol) and Cs₂CO₃ (707.73 g, 2.17 mol) in DMF (2 L) was added dimethyl 1H-pyrazole-3,5-dicarboxylate (231.3 g, 814.56 mmol) and the reaction mixture stirred at 20° C. for 12 h. The reaction was filtered, the filtrate was concentrated in vacuo, the residue was partitioned between DCM (600 mL) and water (500 mL) and extracted with EtOAc (3×200 mL). The combined organic layers were washed with brine (200 mL), dried over Na₂SO₄, filtered and concentrated. The residue was purified by column chromatography (10% to 50%, EtOAc in PE) to give dimethyl 1-(2-(1-methyl-1H-pyrazol-4-yl)-2-oxoethyl)-1H-pyrazole-3,5-dicarboxylate (115 g, 69%) as a brown solid. LCMS m/z=307.1 [M+H]⁺

### Preparation 103

### 4-hydroxy-6-(1-methyl-1H-pyrazol-4-yl)pyrazolo[1,5-a]pyrazine-2-carboxylic acid

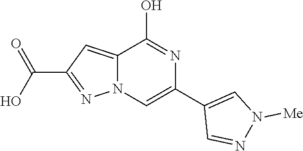 [SMILES: Cn1cc(-c2cn3nc(C(=O)O)cc3c(O)n2)cn1]

Part A: To a solution of dimethyl 1-(2-(1-methyl-1H-pyrazol-4-yl)-2-oxoethyl)-1H-pyrazole-3,5-dicarboxylate (Preparation 102, 100 g, 326.51 mmol) in EtOH (1000 mL) was added ammonium acetate (125.84 g, 1.63 mol) and the reaction stirred for 72 h at 130° C. under high pressure. The cooled reaction was diluted water (500 mL) and the mixture filtered. The filter cake was washed with water (4×100 mL) and evaporated under reduced pressure to give methyl 4-hydroxy-6-(1-methyl-1H-pyrazol-4-yl)pyrazolo[1,5-a]pyrazine-2-carboxylate (85 g, crude) as an off-white solid.

Part B: To a suspension of methyl 4-hydroxy-6-(1-methyl-1H-pyrazol-4-yl)pyrazolo[1,5-a]pyrazine-2-carboxylate (60 g, 219.58 mmol) in water (600 mL) was added sulfuric acid (150 mL) and the reaction stirred at 90° C. for 12 h. The cooled mixture was filtered, the filter cake was washed with water (300 mL) and dried to give 4-hydroxy-6-(1-methyl-1H-pyrazol-4-yl)pyrazolo[1,5-a]pyrazine-2-carboxylic acid (55 g, 97%) as an off-white solid. ¹HNMR (400 MHz, DMSO-d₆) δ: 11.65 (br s, 1H), 8.29 (s, 1H), 8.13 (s, 1H), 8.04 (s, 1H), 7.32 (s, 1H), 3.87 (s, 3H).

### Preparation 104

### 6-(1-methyl-1H-pyrazol-4-yl)pyrazolo[1,5-a]pyrazin-4-ol

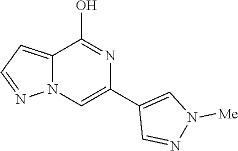 [SMILES: Cn1cc(-c2cn3nccc3c(O)n2)cn1]

4-Hydroxy-6-(1-methyl-1H-pyrazol-4-yl)pyrazolo[1,5-a]pyrazine-2-carboxylic acid (Preparation 103, 5 g, 19.29 mmol) was dissolved in thiolane 1,1-dioxide (70 mL) and stirred at 300° C. for 2 h. The cooled mixture was purified by column chromatography (SiO₂, 10-100%, EtOAc/PE; and 1-10% MeOH/DCM) to give 6-(1-methyl-1H-pyrazol-4-yl)pyrazolo[1,5-a]pyrazin-4-ol (4.2 g, 50%) as a white solid. LCMS m/z=216.2 [M+H]⁺

### Preparation 105

### 4-chloro-6-(1-methyl-1H-pyrazol-4-yl)pyrazolo[1,5-a]pyrazine

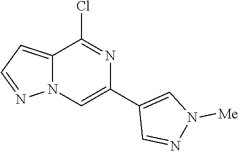 [SMILES: Cn1cc(-c2cn3nccc3c(Cl)n2)cn1]

To a suspension of 6-(1-methyl-1H-pyrazol-4-yl)pyrazolo[1,5-a]pyrazin-4-ol (Preparation 104, 10 g, 46.47 mmol) in MeCN (100 mL) was added phosphoryl trichloride (44 g, 286.96 mmol) and the reaction stirred at 80° C. for 2 h under N₂. The reaction was cooled to 0° C., water (200 mL) added dropwise, and the mixture then neutralized with 2 N NaHCO₃ to pH 8. The mixture was extracted with DCM (3×100 mL). The combined organic layers were washed with brine (100 mL), dried over Na₂SO₄, filtered and concentrated to give 4-chloro-6-(1-methyl-1H-pyrazol-4-yl)pyrazolo[1,5-a]pyrazine (10.5 g, 92.7% yield) as a yellow solid. LCMS m/z=234.1 [M+H]⁺

### Preparation 106

### ethyl 1-cyano-4-methyl-1H-pyrazole-5-carboxylate

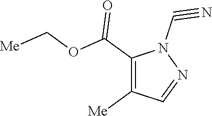 [SMILES: CCOC(=O)c1c(C)cnn1C#N]

2-Chloroacetontirile (13.55 mL, 214.05 mmol) was added dropwise to a mixture of ethyl 4-methyl-1H-pyrazole-5-carboxamide (30 g, 194.60 mmol) and Cs₂CO₃ (76.08 g, 233.51 mmol) in DMF (300 mL) at 20° C. and the mixture stirred at this temperature for 16 h. The mixture was combined with another batch (20 g of ethyl 4-methyl-1H-pyrazole-5-carboxamide). The combined mixture was poured into water (1000 mL), extracted with EtOAc (3×500 mL), the combined organic layer was dried over anhydrous Na₂SO₄, filtered and concentrated in vacuo. The residue was purified by column chromatography (SiO₂, 9% EtOAc/PE) to give ethyl 1-cyano-4-methyl-1H-pyrazole-5-carboxylate (37 g) as a light yellow solid. ¹H NMR (400 MHz, CDCl₃) δ: 7.42 (s, 1H), 5.46 (s, 2H), 4.42 (q, 2H), 2.28 (s, 3H), 1.42 (t, 3H).

### Preparation 107

### ethyl 1-(2-amino-2-oxoethyl)-4-methyl-1H-pyrazole-5-carboxylate

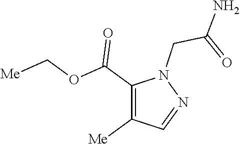 [SMILES: CCOC(=O)c1c(C)cnn1CC(N)=O]

Sulfuric acid (55 mL) was added to a mixture of ethyl 1-cyano-4-methyl-1H-pyrazole-5-carboxylate (Preparation 106, 37 g, 191.51 mmol) in TFA (170 mL) at 25° C. and the mixture stirred at this temperature for 16 h. The mixture was concentrated and the residue was added dropwise to an ice-bath. The mixture was extracted with DCM (3×200 mL), the combined organic layer was dried over anhydrous Na₂SO₄, filtered and concentrated to give ethyl 1-(2-amino-2-oxoethyl)-4-methyl-1H-pyrazole-5-carboxylate (40 g, 99%) as a white solid. ¹H NMR (400 MHz, CDCl₃): 8.70 (br s, 2H), 7.50 (s, 1H), 7.08 (br s, 1H), 6.01 (br s, 1H), 5.31 (s, 2H), 4.39 (q, 2H), 2.30 (s, 3H), 1.39 (t, 3H).

### Preparation 108

### 3-methylpyrazolo[1,5-a]pyrazine-4,6-diol

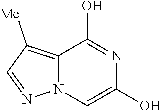 [SMILES: Cc1cnn2cc(O)nc(O)c12]

t-BuONa (43.68 g, 454.51 mmol) was added to a mixture of ethyl 1-(2-amino-2-oxoethyl)-4-methyl-1H-pyrazole-5-carboxylate (Preparation 107, 40 g, 189.38 mmol) in EtOH (4 L) at 25° C. and the mixture stirred at 70° C. for 14 h. The mixture was cooled to 0° C., HCl (con.) was added to adjust the pH to 6-7, and the mixture evaporated under reduced pressure to give 3-methylpyrazolo[1,5-a]pyrazine-4,6-diol (53 g, crude) as a white solid. ¹H NMR (400 MHz, DMSO-d₆) δ: 11.68 (br.s, 1H), 7.57 (s, 1H), 5.09 (s, 2H), 2.26 (s, 3H).

### Preparation 109

### 4,6-dichloro-3-methylpyrazolo[1,5-a]pyrazine

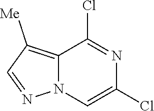 [SMILES: Cc1cnn2cc(Cl)nc(Cl)c12]

The 3-methylpyrazolo[1,5-a]pyrazine-4,6-diol (Preparation 108, 20 g, 121.10 mmol) was added to phosphoryl trichloride (100 mL) at 20° C., then pyridine hydrochloride (13.99 g, 121.10 mmol) was added, the mixture was stirred at 120° C. for 16 hours. The mixture was added to water (200 mL) dropwise, extracted with DCM (3×100 mL), the combined organic layer was dried over anhydrous Na₂SO₄, filtered and concentrated, the residue was purified by column chromatography (SiO2, 16% EtOAc/PE) to give 4,6-dichloro-3-methylpyrazolo[1,5-a]pyrazine (5.6 g, 23%) as a light yellow solid. ¹H NMR (400 MHz, CDCl₃). 8.31 (s, 1H), 7.84 (s, 1H), 2.56 (s, 3H).

### Preparation 110

### (3R,4S)-1-(6-chloropyrazolo[1,5-a]pyrazin-4-yl)-3-cyclopropyl-4-methyl-2-oxopyrrolidine-3-carbonitrile

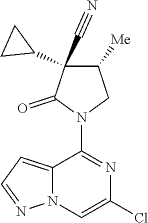 [SMILES: C[C@@H]1CN(c2nc(Cl)cn3nccc23)C(=O)[C@]1(C#N)C1CC1]

(3R,4S)-3-cyclopropyl-4-methyl-2-oxopyrrolidine-3-carbonitrile (Preparation 21, 157.2 mg, 0.957 mmol) and NaH (31.9 mg, 0.798 mmol, 60% purity) in DMF (10 mL) was stirred for 5 min at 25 C. 4,6-Dichloropyrazolo[1,5-a]pyrazine (150.0 mg, 0.798 mmol) was added and the reaction stirred for 20 min. The mixture was concentrated ° in vacuo and the crude product, was purified by column chromatography (10-20% EtOAc/PE) to give (3R,4S)-1-(6-chloropyrazolo[1,5-a]pyrazin-4-yl)-3-cyclopropyl-4-methyl-2-oxopyrrolidine-3-carbonitrile (205 mg, 81.4%) as a white solid. ¹H NMR (500 MHz, CDCl₃) δ: 8.37 (s, 1H), 8.02 (d, 1H), 7.18 (s, 1H), 4.22-4.18 (m, 1H), 3.94-3.87 (m, 1H), 3.11-3.06 (m, 1H), 1.42 (d, 3H), 1.19-1.16 (m, 1H), 0.82-0.72 (m, 4H).

### Preparation 111

### (3R,4S)-1-(6-chloro-3-methylpyrazolo[1,5-a]pyrazin-4-yl)-3-cyclopropyl-4-methyl-2-oxopyrrolidine-3-carbonitrile

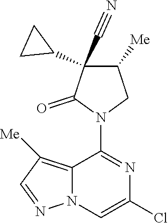 [SMILES: Cc1cnn2cc(Cl)nc(N3C[C@@H](C)[C@@](C#N)(C4CC4)C3=O)c12]

(3R,4S)-1-(6-chloro-3-methylpyrazolo[1,5-a]pyrazin-4-yl)-3-cyclopropyl-4-methyl-2-oxopyrrolidine-3-carbonitrile was obtained as white crystals from 4,6-dichloro-3-methylpyrazolo[1,5-a]pyrazine (Preparation 109) and (3R,4S)-3-cyclopropyl-4-methyl-2-oxopyrrolidine-3-carbonitrile (Preparation 21), following a similar procedure to that described in Preparation 110. ¹H NMR (400 MHz, CDCl₃) δ: 8.31 (s, 1H), 7.84 (s, 1H), 4.07-4.03 (m, 1H), 3.94-3.89 (m, 1H), 3.19-3.09 (m, 1H), 2.36 (s, 3H), 1.43 (d, 3H), 1.22-1.19 (m, 1H), 0.83-0.73 (m, 4H).

### Preparation 112

### 3-(4,4,5,5-tetramethyl-1,3,2-dioxaborolan-2-yl)-6,7-dihydro-4H-pyrazolo[5,1-c][1,4]oxazine

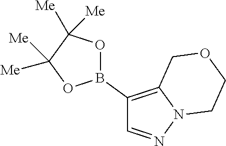 [SMILES: CC1(C)OB(c2cnn3c2COCC3)OC1(C)C]

To a solution of 3-bromo-6,7-dihydro-4H-pyrazolo[5,1-c][1,4]oxazine (100.0 mg, 0.493 mmol) in dioxane (8.0 mL) was added (Bpin)₂ (375.2 mg, 1.48 mmol), Pd(dppf)Cl₂ (36.0 mg, 49.25 umol) and KOAc (145.0 mg, 1.48 mmol) and the reaction stirred at 90° C. for 16 h under N₂. The mixture was diluted with water (10 mL) and extracted with EtOAc (15 mL×3). The combined organic phase was washed with brine (20 mL), dried over anhydrous Na₂SO₄, filtered and concentrated. The crude was purified by flash column (PE/EtOAc=1/0 to 2/1) to give 3-(4,4,5,5-tetramethyl-1,3,2-dioxaborolan-2-yl)-6,7-dihydro-4H-pyrazolo[5,1-c][1,4]oxazine (53.3 mg, crude) as colorless oil. ¹H NMR (400 MHz, CDCl₃) δ ppm: 7.76 (s, 1H), 4.97 (s, 2H), 4.22-4.19 (m, 4H), 1.30 (s, 12H).

### Preparation 113

### 6-(4,4,5,5-tetramethyl-1,3,2-dioxaborolan-2-yl)-3,4-dihydro-1,8-naphthyridin-2(1H)-one

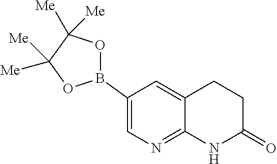 [SMILES: CC1(C)OB(c2cnc3c(c2)CCC(=O)N3)OC1(C)C]

6-(4,4,5,5-tetramethyl-1,3,2-dioxaborolan-2-yl)-3,4-dihydro-1,8-naphthyridin-2(1H)-one was obtained as a yellow solid, 150 mg, 62.1% yield, from 6-bromo-3,4-dihydro-1,8-naphthyridin-2(1H)-one following the procedure described in Preparation 112. LCMS m/z=275.1 [M+H]⁺

### Preparation 114

### 5-(4,4,5,5-tetramethyl-1,3,2-dioxaborolan-2-yl)-1,3-dihydro-2H-pyrrolo[2,3-b]pyridin-2-one

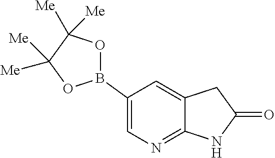 [SMILES: CC1(C)OB(c2cnc3c(c2)CC(=O)N3)OC1(C)C]

5-(4,4,5,5-Tetramethyl-1,3,2-dioxaborolan-2-yl)-1,3-dihydro-2H-pyrrolo[2,3-b]pyridin-2-one was obtained as a yellow solid (500 mg, 65.3% yield) from 5-bromo-1,3-dihydro-2H-pyrrolo[2,3-b]pyridin-2-one, following the procedure described in Preparation 112. LCMS m/z=261.0 [M+H]⁺

### Preparation 115

### 1-(2,2-difluorocyclopropyl)-4-(4,4,5,5-tetramethyl-1,3,2-dioxaborolan-2-yl)-1H-pyrazole

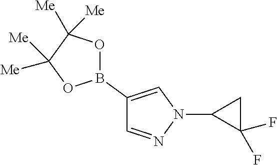 [SMILES: CC1(C)OB(c2cnn(C3CC3(F)F)c2)OC1(C)C]

1-(2,2-difluorocyclopropyl)-4-(4,4,5,5-tetramethyl-1,3,2-dioxaborolan-2-yl)-1H-pyrazole was obtained as a yellow oil (40 mg, 33%) from 4-bromo-1-(2,2-difluorocyclopropyl)-1H-pyrazole (Preparation 132), following the procedure described in Preparation 112. LCMS m/z=270.8 [M+H]⁺.

### Preparation 116

### 1-(difluoromethyl)-5-(4,4,5,5-tetramethyl-1,3,2-dioxaborolan-2-yl)pyridin-2(1H)-one

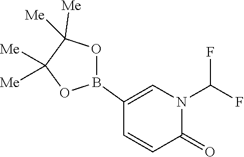 [SMILES: CC1(C)OB(c2ccc(=O)n(C(F)F)c2)OC1(C)C]

The title compound was prepared as a yellow solid (200 mg, 74%) from 5-bromo-1-(difluoromethyl)pyridin-2(1H)-one using an analogous method to that described for Preparation 112. LCMS m/z=272.1 [M+H]⁺.

### Preparation 117

### 1-(methyl-d3)-4-(4,4,5,5-tetramethyl-1,3,2-dioxaborolan-2-yl)-1H-pyrazole

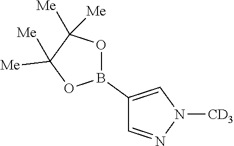 [SMILES: [2H]C([2H])([2H])n1cc(B2OC(C)(C)C(C)(C)O2)cn1]

To a solution of 4-(4,4,5,5-tetramethyl-1,3,2-dioxaborolan-2-yl)-1H-pyrazole (300.0 mg, 1.55 mmol) in DMF (5.0 mL) was added NaH (92.8 mg, 2.32 mmol, 60% purity) at 0° C. and the mixture then stirred at 25° C. for 30 min. CD₃I (268.9 mg, 1.86 mmol) was added and the reaction stirred at 25° C. for 1 h. The mixture was quenched with water (10 mL) slowly, extracted with EtOAc (10 mL×3). The combined organic phase was washed with brine (30 mL), dried over anhydrous Na₂SO₄, filtered and concentrated. The crude was purified by flash column (PE/EtOAc=1/0 to 10/1) to give 1-(methyl-d3)-4-(4,4,5,5-tetramethyl-1,3,2-dioxaborolan-2-yl)-1H-pyrazole (272.3 mg, 83.4%) as colourless oil. LCMS m/z=212.0 [M+H]⁺

### Preparation 118

### tert-butyl 5-bromo-2,3-dihydro-1H-pyrrolo[2,3-b]pyridine-1-carboxylate

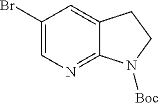 [SMILES: CC(C)(C)OC(=O)N1CCc2cc(Br)cnc21]

To a solution of 5-bromo-2,3-dihydro-1H-pyrrolo[2,3-b]pyridine (1 g, 5.02 mmol) in DCM (50 mL) was added TEA (1.53 g, 15.07 mmol and tert-butoxycarbonyl tert-butyl carbonate (2.19 g, 10.05 mmol) and the mixture stirred at 20° C. for 18 h. The reaction mixture was evaporated to dryness in vacuo and the residue purified by column chromatography (SiO₂, 10% EtOAc/PE) to give tert-butyl 5-bromo-2,3-dihydropyrrolo[2,3-b]pyridine-1-carboxylate as an orange solid (0.54 g, 36%) which was used without further purification.

### Preparation 119

### (1-(tert-butoxycarbonyl)-2,3-dihydro-1H-pyrrolo[2,3-b]pyridin-5-yl)boronic acid

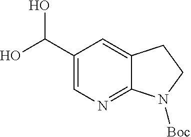 [SMILES: CC(C)(C)OC(=O)N1CCc2cc(C(O)O)cnc21]

Part 1. To a solution of 5-bromo-2,3-dihydro-1H-pyrrolo[2,3-b]pyridine (1 g, 5.02 mmol) in DCM (50 mL) was added TEA (1.53 g, 15.07 mmol and tert-butoxycarbonyl tert-butyl carbonate (2.19 g, 10.05 mmol) and the mixture stirred at 20° C. for 18 h. The reaction mixture was evaporated to dryness in vacuo and the residue purified by column chromatography (SiO₂, 10% EtOAc/PE) to give tert-butyl 5-bromo-2,3-dihydropyrrolo[2,3-b]pyridine-1-carboxylate as an orange solid (0.54 g, 36%) which was used without further purification in Part 2.

Part 2. To a solution of tert-butyl 5-bromo-2,3-dihydro-1H-pyrrolo[2,3-b]pyridine-1-carboxylate (0.54 g, 1.81 mmol) and (Bpin)₂ (687 mg, 2.71 mmol) in dioxane (20 mL) was added KOAc (531 mg, 5.42 mmol), then Pd(dppf)Cl₂ (132 mg, 0.181 mmol) and the reaction stirred at 90° C. under N₂ for 12 h. The mixture was concentrated under vacuum to give (1-(tert-butoxycarbonyl)-2,3-dihydro-1H-pyrrolo[2,3-b]pyridin-5-yl)boronic acid (0.4 g, crude) as a black solid which was used without further purification. LCMS m/z=265.1 [M+H]⁺

### Preparation 120

### 1-(bicyclo[1.1.1]pentan-1-yl)-1H-pyrazole

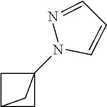

To a solution of compound bicyclo[1.1.1]pentan-1-ylhydrazine (240 mg, 1.40 mmol, 2HCl) in EtOH (8 mL) was added 1,1,3,3-tetramethoxypropane (253 mg, 1.54 mmol) and conc. HCl (163 mg, 4.49 mmol) and the mixture stirred at 80° C. for 16 h. The mixture was extracted with DCM (3×20 mL) and the combined organics washed with brine (30 mL), dried (Na₂SO₄) and concentrated under reduced pressure to give 1-(1-bicyclo[1.1.1]pentanyl)pyrazole as a brown oil (130 mg, 69.%) which was used without further purification. LCMS m/z=135.3 [M+H]⁺

### Preparation 121

### 1-(bicyclo[1.1.1]pentan-1-yl)-4-iodo-1H-pyrazole

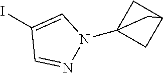 [SMILES: Ic1cnn(C23CC(C2)C3)c1]

To a solution of 1-(bicyclo[1.1.1]pentan-1-yl)-1H-pyrazole (Preparation 120, 120 mg, 0.894 mmol) in AcOH (5 mL) was added NIS (201 mg, 0.894 mmol) and the resulting mixture stirred at 80° C. for 2 h. The mixture was concentrated under reduced pressure and the residue diluted with H₂O (20 mL) and extracted with DCM (3×20 mL). The combined organics were washed with brine (30 mL), dried (Na₂SO₄) and evaporated to dryness in vacuo. The residue was purified by column chromatography (SiO₂, 9-16% EtOAc/PE) to give 1-(1-bicyclo[1.1.1]pentan-1-yl)-4-iodo-1H-pyrazole as a pink oil (90 mg, 39%). LCMS m/z=261.1 [M+H]⁺

### Preparation 122

### 5-methoxy-2-(tributylstannyl)pyridine

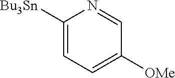

To a solution of 2-bromo-5-methoxy-pyridine (1 g, 5.32 mmol) in THE (10 mL) was added n-BuLi (2.5 M, 2.55 mL) at −70° C. After stirring for 30 min tributyl(chloro)stannane (2.08 g, 6.38 mmol, 1.73 mL) was added and the mixture warmed to 20° C. and stirred for 2 h. The mixture was quenched by excess aqueous KF and extracted with EtOAc (3×20 mL). The combined organics were washed with brine (20 mL) dried (Na₂SO₄) and evaporated to dryness in vacuo. The residue was purified by column chromatography (SiO₂, 9-16% EtOAc/PE) to give 5-methoxy-2-(tributylstannyl)pyridine as a colourless oil (0.35 g, 17%). LCMS m/z=400.1 [M+H]⁺

### Preparation 123

### 3,4-dichloro-6-(1-methyl-1H-pyrazol-4-yl)pyrazolo[1,5-a]pyrazine

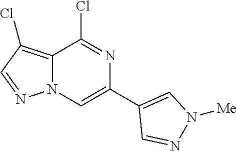 [SMILES: Cn1cc(-c2cn3ncc(Cl)c3c(Cl)n2)cn1]

To a solution of 4-chloro-6-(1-methyl-1H-pyrazol-4-yl)pyrazolo[1,5-a]pyrazine (Preparation 105, 250.0 mg, 1.07 mmol) in DMF (5 mL) was added NCS (142.9 mg, 1.07 mmol) and the reaction was stirred at 50° C. for 2 h. The mixture was quenched with H₂O (20 mL) and extracted with EtOAc (20 mL×2). The combined layers were washed with brine (20 mL), dried over Na₂SO₄, filtered and concentrated under vacuum. The crude was purified by flash column (16-50% EtOAc/PE) to give 3,4-dichloro-6-(1-methyl-1H-pyrazol-4-yl)pyrazolo[1,5-a]pyrazine (180 mg, 62.8%) as a yellow solid. ¹H NMR (500 MHz, CDCl₃) δ ppm: 8.38 (s, 1H), 7.94 (s, 1H), 7.92 (s, 1H), 7.87 (s, 1H), 3.97 (s, 3H).

### Preparation 124

### 4,6-dichloro-3-fluoropyrazolo[1,5-a]pyrazine

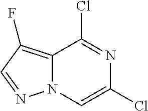 [SMILES: Fc1cnn2cc(Cl)nc(Cl)c12]

To a solution of Selectfluor™ (367.1 g, 1.03 mol) in MeCN (1500 mL) and AcOH (150 mL) was added 4,6-dichloropyrazolo[1,5-a]pyrazine (150 g, 798 mmol) at 20° C. and the resulting mixture stirred at 100° C. for 72 h. The solvent was removed by evaporation and the was diluted with water (500 mL) and extracted with DCM (2×500 mL). The combined organics were washed with brine (500 mL), dried (Na₂SO₄) and evaporated to dryness in vacuo. The residue was purified by column chromatography (SiO₂, 0-50%, EtOAc/PE) to give a solid which was further purified by prep-HPLC-5 (gradient: 40-70%) to give 4,6-dichloro-3-fluoropyrazolo[1,5-a]pyrazine as an off-white solid (30.2 g, 18%). LCMS m/z=205.9 [M+H]⁺

### Preparation 125

### (3R,4S)-1-(6-chloro-3-fluoropyrazolo[1,5-a]pyrazin-4-yl)-3-cyclopropyl-4-methyl-2-oxopyrrolidine-3-carbonitrile

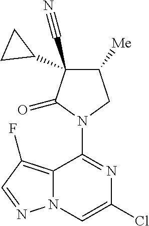 [SMILES: C[C@@H]1CN(c2nc(Cl)cn3ncc(F)c23)C(=O)[C@]1(C#N)C1CC1]

To a solution of 4,6-dichloro-3-fluoropyrazolo[1,5-a]pyrazine (63.1 mg, 0.384 mmol) and NaH (12.82 mg, 0.320 mmol, 60% purity) in DMF (3.0 mL) was stirred at 25° C. for 5 min. (3R,4S)-3-cyclopropyl-4-methyl-2-oxopyrrolidine-3-carbonitrile (Preparation 21, 66 mg, 0.320 mmol) was added and the mixture stirred at 25° C. for 20 min. The mixture was concentrated under reduced pressure and purified by column chromatography (33% EtOAc/PE) to give (3R,4S)-1-(6-chloro-3-fluoropyrazolo[1,5-a]pyrazin-4-yl)-3-cyclopropyl-4-methyl-2-oxopyrrolidine-3-carbonitrile (76 mg, 71%) as a white solid. LCMS m/z=334.0 [M+H]⁺.

### Preparation 126

### (3R,4S)-3-cyclopropyl-1-(3-fluoro-6-(tributylstannyl)pyrazolo[1,5-a]pyrazin-4-yl)-4-methyl-2-oxopyrrolidine-3-carbonitrile

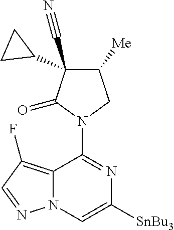

To a solution of (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 200 mg, 0.599 mmol) in toluene (5.0 mL) was added (SnBu₃)₂ (521.5 mg, 0.899 mmol) and Pd(dtbpf)Cl₂ (39.1 mg, 0.060 mmol) under N₂ and the reaction stirred at 100° C. for 20 h. The mixture was added water (10 mL) and extracted with EtOAc (10 mL×3). The combined layers were washed with brine (30 mL), dried over Na₂SO₄, filtered and concentrated under vacuum to give the crude, which was purified by flash column (PE/EtOAc=10/1 to 3/1) to give (3R,4S)-3-cyclopropyl-1-(3-fluoro-6-(tributylstannyl)pyrazolo[1,5-a]pyrazin-4-yl)-4-methyl-2-oxopyrrolidine-3-carbonitrile (70 mg, 19.9% yield) as yellow oil. ¹H NMR (500 MHz, CDCl₃) δ: 7.98 (s, 1H), 7.78 (d, 1H), 4.24-4.17 (m, 1H), 3.77-3.72 (m, 1H), 3.16-3.12 (m, 1H), 1.67-1.61 (m, 6H), 1.42 (d, 3H), 1.37-1.29 (m, 12H), 0.93-0.91 (m, 10H), 0.90-0.75 (m, 4H).

### Preparation 127

### (3S,4R)-1-(6-bromopyrrolo[1,2-b]pyridazin-4-yl)-3-cyclopropyl-4-methyl-2-oxopyrrolidine-3-carbonitrile

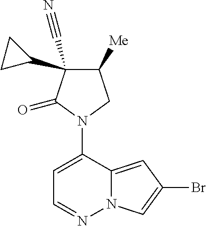 [SMILES: C[C@H]1CN(c2ccnn3cc(Br)cc23)C(=O)[C@@]1(C#N)C1CC1]

To a solution of 6-bromopyrrolo[1,2-b]pyridazin-4-yl trifluoromethanesulfonate (Preparation 65, 100 mg, 0.290 mmol) and (3S,4R)-3-cyclopropyl-4-methyl-2-oxopyrrolidine-3-carbonitrile (Preparation 19, 52.3 mg, 0.319 mmol) in dioxane (5 mL) was added Pd₂(dba)₃ (26.5 mg, 0.029 mmol), Xantphos (16.8 mg, 0.029 mmol) and K₃PO₄ (86.1 mg, 0.406 mmol) at 25° C. The mixture was stirred at 70° C. for 1 h under N₂. The mixture was treated with H₂O (10 mL) and extracted with EtOAc (3×10 mL). The combined organics were washed with brine (20 mL), dried (Na₂SO₄) and evaporated to dryness in vacuo. The residue was purified by column chromatography (SiO₂; 6-25% EtOAc/PE) to give (3S,4R)-1-(6-bromopyrrolo[1,2-b]pyridazin-4-yl)-3-cyclopropyl-4-methyl-2-oxopyrrolidine-3-carbonitrile as a yellow solid (100 mg, 96%). LCMS m/z=360.9 [M+H]⁺; ¹H NMR (500 MHz, CDCl₃) δ: 8.01 (d, 1H), 7.79 (d, 1H), 6.95 (d, 1H), 6.61 (d, 1H), 4.09-4.06 (m, 1H), 3.85-3.80 (m, 1H), 3.10-3.03 (m, 1H), 1.42 (d, 3H), 1.18-1.13 (m, 1H), 0.81-0.76 (m, 4H).

### Preparation 128

### 4-(tert-butyl) 1-ethyl 2-cyano-2-cyclopropyl-3-methylenesuccinate

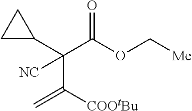

To a solution of ethyl 2-cyano-2-cyclopropylacetate (Preparation 4, 6.7 g, 43.7 mmol) in THF (10 mL) was added tert-butyl propiolate (6.07 g, 48.1 mmol) and Pd(PPh₃)₄ (1.52 g, 1.31 mmol) and the mixture was stirred at 25° C. for 24 h under N₂. The mixture was evaporated to dryness in vacuo and the residue purified by column chromatography (SiO₂, 0-10% EtOAc/PE) to give 4-(tert-butyl) 1-ethyl 2-cyano-2-cyclopropyl-3-methylenesuccinate as a brown oil (9.6 g, 79%). ¹H NMR (500 MHz, CDCl₃) δ: 6.65 (s, 1H), 6.23 (s, 1H), 4.32-4.24 (m, 2H), 1.50 (s, 9H), 1.38-1.29 (m, 4H), 0.87-0.85 (m, 2H), 0.73-0.68 (m, 2H).

### Preparation 129

### tert-butyl 4-cyano-4-cyclopropyl-1-(4-methoxybenzyl)-5-oxopyrrolidine-3-carboxylate

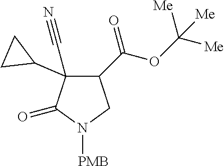

To a solution of 4-(tert-butyl) 1-ethyl 2-cyano-2-cyclopropyl-3-methylenesuccinate (Preparation 128, 9.6 g, 34.4 mmol) in EtOH (100 mL) was added PMBNH₂ (7.07 g, 51.6 mmol) and TEA (6.96 g, 68.7 mmol) and the mixture was stirred at 80° C. for 20 h. The mixture was evaporated to dryness in vacuo and the residue was purified by column chromatography (0-25% EtOAc/PE) to give tert-butyl 4-cyano-4-cyclopropyl-1-(4-methoxybenzyl)-5-oxopyrrolidine-3-carboxylate as a brown oil (9.5 g, 75%). LCMS m/z=371.3 [M+H]⁺;

### Preparation 130

### 4-cyano-4-cyclopropyl-1-(4-methoxybenzyl)-5-oxopyrrolidine-3-carboxylic acid

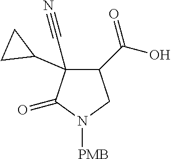

To a solution of tert-butyl 4-cyano-4-cyclopropyl-1-(4-methoxybenzyl)-5-oxopyrrolidine-3-carboxylate (Preparation 129, 1.0 g, 2.70 mmol) in DCM (10 mL) was added 2,6-Lutidine (5.8 g, 54 mmol, 6.29 mL) and TMSOTf (6.0 g, 27 mmol, 4.89 mL) at 20° C. and the mixture stirred at 20° C. for 24 h. The mixture was concentrated under reduced pressure and the residue was purified by prep-HPLC-6 (gradient: 23-53%) to give 4-cyano-4-cyclopropyl-1-(4-methoxybenzyl)-5-oxopyrrolidine-3-carboxylic acid as a yellow oil (830 mg, 98%).

### Preparation 131

### S-ethyl 4-cyano-4-cyclopropyl-1-(4-methoxybenzyl)-5-oxopyrrolidine-3-carbothioate

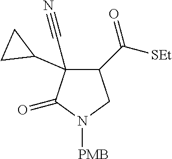

To a solution of 4-cyano-4-cyclopropyl-1-(4-methoxybenzyl)-5-oxopyrrolidine-3-carboxylic acid (Preparation 130, 830 mg, 2.64 mmol) in DCM (10 mL) was added DMAP (41.5 mg, 0.340 mmol) and DCC (817.2 mg, 3.96 mmol) and EtSH (410.2 mg, 6.60 mmol) at 20° C. and the mixture stirred at 20° C. for 4 h. The mixture was concentrated and purified by column chromatography (SiO₂; 25-50% EtOAc/PE/) to give S-ethyl 4-cyano-4-cyclopropyl-1-(4-methoxybenzyl)-5-oxopyrrolidine-3-carbothioate as a light yellow oil (418 mg, 44%). LCMS m/z=358.8 [M+H]⁺.

### Preparation 132

### 3-cyclopropyl-4-formyl-1-(4-methoxybenzyl)-2-oxopyrrolidine-3-carbonitrile

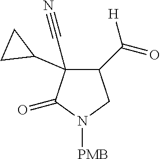

To a solution of S-ethyl 4-cyano-4-cyclopropyl-1-(4-methoxybenzyl)-5-oxopyrrolidine-3-carbothioate (Preparation 131, 410 mg, 1.14 mmol) in DCM (10 mL) was added Pd/C (121.7 mg, 10 mol %, 10% purity) and Et₃SiH (399 mg, 3.43 mmol) at 25° C. and the mixture was stirred at 25° C. for 3 h. The mixture was concentrated under reduced pressure and the residue was purified by-TLC (SiO₂; 50% PE/EtOAc) to give 3-cyclopropyl-4-formyl-1-(4-methoxybenzyl)-2-oxopyrrolidine-3-carbonitrile as a yellow gum (300 mg, 88%). LCMS m/z=316.7 [M+H]⁺.

### Preparation 133

### 3-cyclopropyl-4-(difluoromethyl)-1-(4-methoxybenzyl)-2-oxopyrrolidine-3-carbonitrile

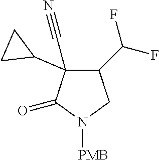

DAST (3.7 g, 22.7 mmol, 3 mL) was added to a solution of 3-cyclopropyl-4-formyl-1-(4-methoxybenzyl)-2-oxopyrrolidine-3-carbonitrile (Preparation 132, 300 mg, 1.01 mmol) in DCM (3 mL) at 25° C. and the mixture stirred at 25° C. for 16 h. The reaction was quenched with H₂O (10 mL) and extracted with EtOAc (3×5 mL). The combined organics were washed with brine (10 mL), dried (Na₂SO₄) and evaporated to dryness in vacuo and the residue purified by column chromatography (SiO₂; 25-50% EtOAc/PE) to give 3-cyclopropyl-4-(difluoromethyl)-1-(4-methoxybenzyl)-2-oxopyrrolidine-3-carbonitrile as a yellow solid (140 mg, 43%). LCMS m/z=320.7 [M+H]⁺.

### Preparation 134

### 3-cyclopropyl-4-(difluoromethyl)-2-oxopyrrolidine-3-carbonitrile

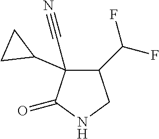 [SMILES: N#CC1(C2CC2)C(=O)NCC1C(F)F]

CAN (513.4 mg, 0.937 mmol) was added to a solution of 3-cyclopropyl-4-(difluoromethyl)-1-(4-methoxybenzyl)-2-oxopyrrolidine-3-carbonitrile (Preparation 133, 100 mg, 0.312 mmol) in MeCN (10 mL) and H₂O (2 mL) at 25° C. and the resulting mixture stirred at 25° C. for 24 h. The reaction was diluted with H₂O (10 mL) and extracted with EtOAc (3×5 mL). The combined organics were washed with brine (10 mL), dried (Na₂SO₄) and evaporated to dryness in vacuo. The residue was purified by-TLC (50% PE/EtOAc) to give 3-cyclopropyl-4-(difluoromethyl)-2-oxopyrrolidine-3-carbonitrile as a colourless oil (60 mg, 96%). ¹H NMR (400 MHz, CDCl₃) δ: 6.35-5.98 (m, 2H), 3.63-2.92 (m, 3H), 1.25-1.22 (m, 1H), 0.88-0.75 (m, 4H).

### Preparation 135

### 1-(6-bromopyrrolo[1,2-b]pyridazin-4-yl)-3-cyclopropyl-4-(difluoromethyl)-2-oxopyrrolidine-3-carbonitrile

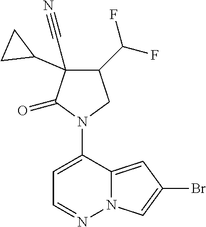 [SMILES: N#CC1(C2CC2)C(=O)N(c2ccnn3cc(Br)cc23)CC1C(F)F]

To a solution of 6-bromopyrrolo[1,2-b]pyridazin-4-yl trifluoromethanesulfonate (Preparation 65, 94.0 mg, 0.272 mmol) and 3-cyclopropyl-4-(difluoromethyl)-2-oxopyrrolidine-3-carbonitrile (Preparation 134, 60.0 mg, 0.300 mmol) in dioxane (10 mL) was added K₃PO₄ (81.0 mg, 0.381 mmol), Xantphos (15.8 mg, 27.3 □mol) and Pd₂dba₃ (25.0 mg, 27.3 □mol) at 25° C. The mixture was stirred at 70° C. for 16 hours under N₂. The mixture was evaporated to dryness in vacuo and the residue was purified by column chromatography (SiO₂, 6-25% EtOAc/PE) to give 1-(6-bromopyrrolo[1,2-b]pyridazin-4-yl)-3-cyclopropyl-4-(difluoromethyl)-2-oxopyrrolidine-3-carbonitrile as a light yellow solid (63 mg, 59%). LCMS m/z=395.0 [M+H]⁺.

### Preparation 136

### 1-(2,2-difluorocyclopropyl)-1H-pyrazole

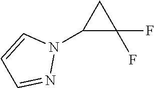

NaI (558 mg, 3.72 mmol) was added to a solution of 1-vinyl-1H-pyrazole (1.0 g, 10.63 mmol) in THE (8.0 mL) under N₂ and the mixture was heated to 70° C. TMSCF₃ (5.3 g, 37.2 mmol) was added dropwise at 70° C. and the resulting mixture stirred at 70° C. for 16 h. The reaction mixture was evaporated to dryness in vacuo and the residue diluted with DCM (50 ml) and the precipitate removed by filtration and discarded. The combined filtrates were evaporated to dryness in vacuo to afford 1-(2,2-difluorocyclopropyl)-1H-pyrazole as a brown oil (1.2 g, 78%). LCMS m/z=145.2 [M+H]⁺.

### Preparation 137

### 4-bromo-1-(2,2-difluorocyclopropyl)-1H-pyrazole

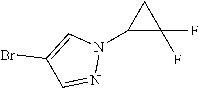 [SMILES: FC1(F)CC1n1cc(Br)cn1]

Br₂ (0.333 mg, 2.08 □mol) was added to a solution of 1-(2,2-difluorocyclopropyl)-1H-pyrazole (Preparation 136, 200 mg, 1.39 mmol) in DCM (5 mL) at 10° C. under N₂ and the mixture stirred at 25° C. for 1.5 h. The solution was quenched with 10% aq. Na₂SO₃ (50 mL). The organics were separated, washed with brine (50 mL), dried (Na₂SO₄) and evaporated to dryness in vacuo to afford 4-bromo-1-(2,2-difluorocyclopropyl)-1H-pyrazole as a brown oil (280 mg, 90%). ¹H NMR (400 MHz, CDCl₃) δ: 7.53 (s, 1H), 7.51 (s, 1H), 4.08-4.03 (m, 1H), 2.23-2.19 (m, 1H), 2.11-2.05 (m, 1H).

### Preparation 138

### 3-(4-bromo-1H-pyrazol-1-yl)cyclobutan-1-one

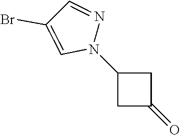 [SMILES: O=C1CC(n2cc(Br)cn2)C1]

To a solution of cyclobutanone-4-bromo-1H-pyrazole (500 mg, 3.40 mmol) in DMF (1 mL) was added NaH (163 mg, 4.08 mmol, 60% purity) at 0° C. The mixture was stirred at 25° C. for 30 min and 3-bromocyclobutan-1-one (557.5 mg, 3.74 mmol) was added. The mixture was stirred at 20° C. for 1 h. The mixture was diluted with water (5 mL) and extracted with EtOAc (3×10 mL). The combined organics were washed with brine (10 mL), dried (Na₂SO₄) and evaporated to dryness in vacuo. The residue was purified by flash chromatography (SiO2, 0-25% EtOAc/PE) to give 3-(4-bromo-1H-pyrazol-1-yl)cyclobutan-1-one as a white solid (80 mg, 11%). ¹H NMR (400 MHz, CDCl₃) δ: 7.55 (s, 2H), 5.03-4.96 (m, 1H), 3.81-3.74 (m, 2H), 3.60-3.53 (m, 2H).

### Preparation 139

### 4-bromo-1-(3,3-difluorocyclobutyl)-1H-pyrazole

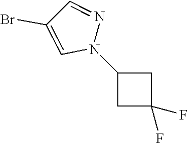 [SMILES: FC1(F)CC(n2cc(Br)cn2)C1]

DAST (75 mg, 61.44 uL) was added to a solution of 3-(4-bromo-1H-pyrazol-1-yl)cyclobutan-1-one (Preparation 138, 50 mg, 0.233 mmol) in DCM (2 mL) and the mixture was stirred at 25° C. for 16 h. The mixture was diluted with H₂O (5 mL) and extracted with EtOAc (3×10 mL). The combined organics were washed with brine (30 mL), dried (Na₂SO₄) and evaporated to dryness in vacuo. The residue was purified by prep-TLC (20% EtOAc/PE) to give 4-bromo-1-(3,3-difluorocyclobutyl)-1H-pyrazole as a white solid (22 mg, 40%). ¹H NMR (500 MHz, CDCl₃) δ: 7.53 (s, 1H), 7.49 (s, 1H), 4.71-4.64 (m, 1H), 3.20-3.11 (m, 4H).

### Preparation 140

### 3-(4-bromo-1H-pyrazol-1-yl)pyridine

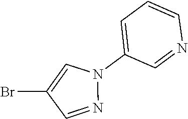 [SMILES: Brc1cnn(-c2cccnc2)c1]

To a solution of 4-bromo-1H-pyrazole (500 mg, 3.40 mmol) and pyridin-3-ylboronic acid (836 mg, 6.80 mmol) in pyridine (25 mL) was added Cu(OAc)₂ (309 mg, 1.70 mmol.) and the mixture was stirred at 100° C. for 16 h. The reaction mixture was concentrated and the residue purified by column chromatography (SiO₂, 0-100% EtOAc/PE) to afford 3-(4-bromo-1H-pyrazol-1-yl)pyridine as a white solid (500 mg, 66%). LCMS m/z=226.1 [M+H]⁺.

### Preparation 141

### 1-(methylsulfonyl)-4-(4,4,5,5-tetramethyl-1,3,2-dioxaborolan-2-yl)-1H-pyrazole

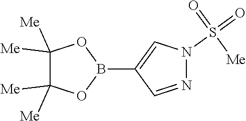 [SMILES: CC1(C)OB(c2cnn(S(C)(=O)=O)c2)OC1(C)C]

To a solution of 4-(4,4,5,5-tetramethyl-1,3,2-dioxaborolan-2-yl)-1H-pyrazole (1 g, 5.15 mmol) in DCM (10 mL) was added TEA (1.6 g, 15.46 mmol) and MsCl (590 mg, 5.15 mmol) at 0° C. and the mixture was stirred at 25° C. for 16 h. The reaction was diluted with H₂O (20 mL). The mixture was extracted with DCM (2×20 mL) and the combined extracts were washed with brine (2×20 mL), dried (Na₂SO₄) and evaporated to dryness in vacuo. The residue was purified by column chromatography (SiO₂, 5-10% EtOAc/PE) to give 1-(methylsulfonyl)-4-(4,4,5,5-tetramethyl-1,3,2-dioxaborolan-2-yl)-1H-pyrazole as a colourless oil (901 mg, 64%). ¹H NMR (500 MHz, CDCl₃) δ: 8.33 (s, 1H), 8.02 (s, 1H), 3.32 (s, 3H), 1.33 (s, 12H).

### Preparation 142

### 1-(2-ethoxyethyl)-4-(4,4,5,5-tetramethyl-1,3,2-dioxaborolan-2-yl)-1H-pyrazole

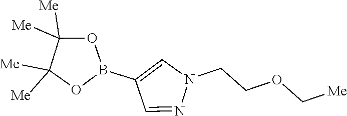 [SMILES: CCOCCn1cc(B2OC(C)(C)C(C)(C)O2)cn1]

A mixture of 4-(4,4,5,5-tetramethyl-1,3,2-dioxaborolan-2-yl)-1H-pyrazole (50 mg, 0.258 mmol), 1-chloro-2-ethoxy-ethane (33.6 mg, 0.309 mmol) and K₂CO₃ (125 mg, 0.902 mol) in DMF (1.5 mL) was stirred at 70° C. overnight. The reaction was diluted with EtOAc, washed with NaHCO₃, H₂O and brine. The combined organics were dried (Na₂SO₄) and evaporated to dryness in vacuo to afford 1-(2-ethoxyethyl)-4-(4,4,5,5-tetramethyl-1,3,2-dioxaborolan-2-yl)-1H-pyrazole which was used without further purification. LCMS m/z=267 [M+H]⁺

### Preparation 143

### 7-chloro-2-(1-methyl-1H-pyrazol-4-yl)furo[3,2-b]pyridine

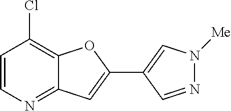 [SMILES: Cn1cc(-c2cc3nccc(Cl)c3o2)cn1]

To a solution of 7-chloro-2-iodofuro[3,2-b]pyridine (400 mg, 1.43 mmol) in dioxane (2.5 mL) and H₂O (0.9 mL) was added 1-methyl-4-(4,4,5,5-tetramethyl-1,3,2-dioxaborolan-2-yl)-1H-pyrazole (313 mg, 1.50 mmol), NaHCO₃ (144 mg, 1.72 mmol) and Pd(dppf)Cl₂ (105 mg, 0.143 mmol) under N₂ and the mixture stirred at 80° C. for 3 h under N₂. The mixture was concentrated under reduced pressure and purified by column chromatography (SiO₂, 0-10% EtOAc/PE) to give 7-chloro-2-(1-methyl-1H-pyrazol-4-yl)furo[3,2-b]pyridine as a brown solid (180 mg, 53%). ¹H NMR (400 MHz, CDCl₃) δ: 8.36 (d, 1H), 7.93 (s, 1H), 7.90 (s, 1H), 7.18 (d, 1H), 6.91 (s, 1H), 4.00 (s, 3H).

### Preparation 144

### methyl 1′-methyl-1′H,2H-[3,4′-bipyrazole]-5-carboxylate

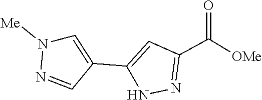 [SMILES: COC(=O)c1cc(-c2cnn(C)c2)[nH]n1]

Part 1: Dimethyl oxalate (3.8 g, 32.2 mmol) and NaOMe (2.6 g, 48.33 mmol) were added to a solution of 1-(1-methyl-1H-pyrazol-4-yl)ethan-1-one (2.0 g, 16.11 mmol) in MeOH (35 mL). The mixture was stirred at 15° C. for 16 h. The mixture was cooled in an ice-water bath and the pH adjusted to ~3 with 2N HCl. The resulting solid was collected and concentrated to give methyl (Z)-2-hydroxy-4-(1-methyl-1H-pyrazol-4-yl)-4-oxobut-2-enoate as a yellow solid (1.8 g, 53% yield) which was used without further purification. ¹H NMR (500 MHz, DMSO-d₆) δ: 8.68 (s, 1H), 8.17 (s, 1H), 6.83 (s, 1H), 3.91 (s, 3H), 3.84 (s, 3H).

Part 2: Hydrazine hydrate (557 mg, 11.1 mmol) was added to a solution of methyl (Z)-2-hydroxy-4-(1-methyl-1H-pyrazol-4-yl)-4-oxobut-2-enoate (Part 1, 1.8 g, 8.56 mmol) in AcOH (8 mL) and the mixture stirred at 100° C. for 2 h. The mixture was cooled to room temperature, adjusted to pH ~7 with aq. NaHCO₃ and extracted with EtOAc (2×50 mL). The combined organics were washed with brine (30 mL), dried (Na₂SO₄) and evaporated to dryness in vacuo. The residue was purified by column chromatography (SiO₂, 15-50% EtOAc/PE) to give methyl 1′-methyl-1′H,2H-[3,4′-bipyrazole]-5-carboxylate as a colourless oil (1.1 g, 62% yield). ¹H NMR (400 MHz, DMSO-d₆) δ: 13.61 (s, 1H), 8.04 (s, 1H), 7.94 (s, 1H), 6.88 (s, 1H), 3.83 (s, 3H), 3.78 (s, 3H).

### Preparation 145.

### 1′-methyl-1′H,2H-[3,4′-bipyrazole]-5-carboxylic acid

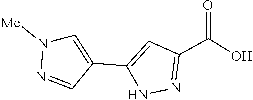 [SMILES: Cn1cc(-c2cc(C(=O)O)n[nH]2)cn1]

To a solution of methyl 1′-methyl-1′H,2H-[3,4′-bipyrazole]-5-carboxylate (Preparation 144, 1.1 g, 5.33 mmol) in THE (10 mL) and water (1 mL) was added NaOH (427 mg, 10.7 mmol) and the mixture stirred at 60° C. for 5 h. Most of solvent was removed and pH adjusted to pH ~3 and the resulting solid collected to give 1′-methyl-1′H,2H-[3,4′-bipyrazole]-5-carboxylic acid as a yellow solid (1 g, 98%). ¹H NMR (500 MHz, DMSO-d₆) δ ppm: 13.18 (s, 1H), 8.06 (s, 1H), 7.81 (s, 1H), 6.86 (s, 1H), 3.87 (s, 3H).

### Preparation 146

### N-(2,2-dimethoxyethyl)-1′-methyl-1′H,2H-[3,4′-bipyrazole]-5-carboxamide

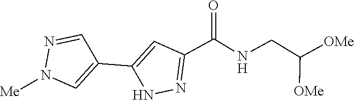 [SMILES: COC(CNC(=O)c1cc(-c2cnn(C)c2)[nH]n1)OC]

To a solution of 1′-methyl-1′H,2H-[3,4′-bipyrazole]-5-carboxylic acid (Preparation 145, 900 mg, 4.68 mmol) in DMF (10 mL) was added HATU (3.57 g, 9.37 mmol). The mixture was stirred 15 min before the addition of TEA (1.42 g, 14.05 mmol, 1.96 mL) and 2,2-dimethoxyethan-1-amine (591 mg, 5.62 mmol) and the reaction mixture stirred at 25° C. for 5 h. The mixture was treated with H₂O (50 mL) and extracted with DCM (3×50 mL). The combined organics were washed with brine (50 mL), dried (Na₂SO₄) and evaporated to dryness in vacuo. The residue was purified by column chromatography (SiO₂, 4-9% MeOH/DCM) to give N-(2,2-dimethoxyethyl)-1′-methyl-1′H,2H-[3,4′-bipyrazole]-5-carboxamide as a white solid (1.1 g, 84%). ¹H NMR (500 MHz, CDCl₃) δ: 7.72-7.69 (m, 2H), 6.79 (s, 1H), 4.58-4.56 (m, 1H), 3.95 (s, 3H), 3.48 (s, 6H), 3.27-3.23 (m, 2H).

### Preparation 147

### 2-(1-methyl-1H-pyrazol-4-yl)pyrazolo[1,5-a]pyrazin-4(5H)-one

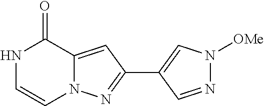 [SMILES: COn1cc(-c2cc3c(=O)[nH]ccn3n2)cn1]

Part A: To a solution of N-(2,2-dimethoxyethyl)-1′-methyl-1′H,2H-[3,4′-bipyrazole]-5-carboxamide (Preparation 146, 1.0 g, 3.58 mmol) in HCl (5M, 15 mL) was stirred at 20° C. for 10 min. The solids were collected by filtration and the filter cake washed with water (20 mL) and EtOH (20 mL), and dried under vacuum to afford 7-hydroxy-2-(1-methyl-1H-pyrazol-4-yl)-6,7-dihydropyrazolo[1,5-a]pyrazin-4(5H)-one as a white solid (805 mg, crude) which was used without further purification.

Part B: A solution of 7-hydroxy-2-(1-methyl-1H-pyrazol-4-yl)-6,7-dihydropyrazolo[1,5-a]pyrazin-4(5H)-one (Part A, 800 mg, 3.43 mmol) in CH₃SO₂H (10 mL) was stirred at 50° C. for 20 h. The reaction mixture was evaporated to dryness and the residue purified by prep-HPLC-12 (gradient: 0-25%) to give 2-(1-methyl-1H-pyrazol-4-yl)pyrazolo[1,5-a]pyrazin-4(5H)-one as a white solid (350 mg, 47%). ¹H NMR (500 MHz, DMSO-d₆) δ ppm: 11.21 (s, 1H), 8.16 (s, 1H), 7.87 (s, 1H), 7.63 (d, 1H), 7.17 (s, 1H), 6.84-8.81 (m, 1H), 3.88 (s, 3H).

### Preparation 148

### 4-chloro-2-(1-methyl-1H-pyrazol-4-yl)pyrazolo[1,5-a]pyrazine

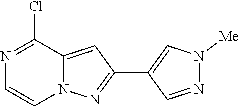 [SMILES: Cn1cc(-c2cc3c(Cl)nccn3n2)cn1]

A solution of 2-(1-methyl-1H-pyrazol-4-yl)pyrazolo[1,5-a]pyrazin-4(5H)-one (Preparation 147, 150 mg, 0.697 mmol) in POCl₃ (15 mL) was stirred at 120° C. for 4 h. The mixture was concentrated under vacuum and the residue was partitioned between EtOAc (20 mL) and water (50 mL) and extracted with EtOAc (3×20 mL). The combined organics were washed with brine (20 mL), (Na₂SO₄) and evaporated to dryness in vacuo to afford 4-chloro-2-(1-methyl-1H-pyrazol-4-yl)pyrazolo[1,5-a]pyrazine as a grey solid (110 mg, 67%) which was used without additional purification. ¹H NMR (500 MHz, CDCl₃) δ: 8.26 (d, 1H), 8.96 (s, 1H), 7.88 (s, 1H), 7.62 (d, 1H), 6.90 (d, 1H), 4.01 (s, 3H).

### Preparation 149

### (3R,4S)-1-(2-bromo-3-fluoroimidazo[1,2-b]pyridazin-8-yl)-3-cyclopropyl-4-methyl-2-oxopyrrolidine-3-carbonitrile

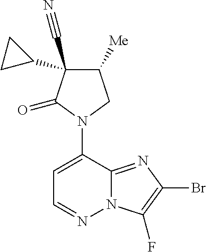 [SMILES: C[C@@H]1CN(c2ccnn3c(F)c(Br)nc23)C(=O)[C@]1(C#N)C1CC1]

A solution of (3R,4S)-1-(2-bromoimidazo[1,2-b]pyridazin-8-yl)-3-cyclopropyl-4-methyl-2-oxopyrrolidine-3-carbonitrile (Preparation 97, 150 mg, 0.416 mmol) and Select-F (147.6 mg, 0.416 mmol) in MeCN (5 mL) was stirred at 0° C. for 2 h and then warmed to 25° C. with stirring for 20 h. The reaction was quenched with water (50 mL) and extracted with EtOAc (3×20 mL). The combined organics were washed with brine (20 mL), dried (Na₂SO₄) and evaporated to dryness in vacuo. The residue was purified by prep-HPLC-3 (Gradient: 48-78%) to afford (3R,4S)-1-(2-bromo-3-fluoroimidazo[1,2-b]pyridazin-8-yl)-3-cyclopropyl-4-methyl-2-oxopyrrolidine-3-carbonitrile as a white solid (20 mg, 13%). LCMS m/z=380.0 [M+H]⁺.

### Preparation 150

### 7-chloro-5-(methylthio)imidazo[1,2-c]pyrimidine

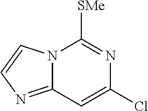 [SMILES: CSc1nc(Cl)cc2nccn12]

2-chloroacetaldehyde (4.5 g, 56.93 mmol) was added to a solution of 6-chloro-2-(methylthio)pyrimidin-4-amine (5.0 g, 28.5 mmol) in dioxane (50 mL) at 25° C. and the resulting mixture was stirred at 95° C. for 16 h. The mixture was evaporated to dryness, diluted with H₂O (40 mL) and extracted with EtOAc (3×20 mL). The combined extracts were washed with brine (50 mL), dried (Na₂SO₄) and evaporated to dryness in vacuo. The residue was purified by column chromatography (SiO₂; 6-16% EtOAc/PE) to give 7-chloro-5-(methylthio)imidazo[1,2-c]pyrimidine as a yellow solid (1.0 g, 18%) as yellow solid. The aqueous phase was concentrated under reduced pressure to give additional 7-chloro-5-(methylthio)imidazo[1,2-c]pyrimidine as an orange solid (3.8 g, crude). LCMS m/z=200.0 [M+H]⁺.

### Preparation 151

### 7-chloroimidazo[1,2-c]pyrimidin-5-ol

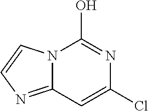 [SMILES: Oc1nc(Cl)cc2nccn12]

A solution of KOH (4.0 g, 70.42 mmol) in H₂O (50 mL) was added slowly to a solution/suspension of 7-chloro-5-(methylthio)imidazo[1,2-c]pyrimidine (Preparation 150, 3.7 g, 18.53 mmol) in MeOH (20 mL) and then stirred at 80° C. for 2 h. The reaction mixture was adjusted to pH 6-7 with 2 N HCl. The solids were collected by filtration and washed with MeOH and dried under reduced pressure to afford 7-chloroimidazo[1,2-c]pyrimidin-5-ol as a grey solid (2 g, 63%). ¹H NMR (400 MHz, DMSO-d₆) δ: 13.16-12.76 (m, 1H), 7.70 (s, 1H), 7.54 (s, 1H), 6.62 (s, 1H).

### Preparation 152

### 7-(1-methyl-1H-pyrazol-4-yl)imidazo[1,2-c]pyrimidin-5-ol

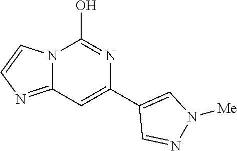 [SMILES: Cn1cc(-c2cc3nccn3c(O)n2)cn1]

To a solution of 7-chloroimidazo[1,2-c]pyrimidin-5-ol (Preparation 151, 1.5 g, 8.85 mmol) and 1-methyl-4-(4,4,5,5-tetramethyl-1,3,2-dioxaborolan-2-yl)-1H-pyrazole (3.7 g, 17.69 mmol) in IPA (20 mL) was added K₃PO₄ (2 M, 13.3 mL), Pd₂(dba)₃ (810 mg, 0.885 mmol) and Xphos (422 mg, 0.885 mmol) at 25° C. and the mixture stirred at 100° C. for 16 h under N₂. The mixture was concentrated under reduce pressure to give an aqueous syrup which was partitioned into H₂O (50 mL) and EtOAc (30 mL). The mixture was filtered and the orange organic phase was removed. The aqueous phase was adjust to pH 6 with 2 N HCl and the mixture was concentrated under reduced pressure to give a yellow solid. The yellow solid was treated with DCM/MeOH (10:1, 50 mL) with stirring for 3 h at 25° C. The resulting solids were collected by filtration, washed with 10:1 DCM/MeOH (2×30 mL) and dried in vacuo to afford 7-(1-methyl-1H-pyrazol-4-yl)imidazo[1,2-c]pyrimidin-5-ol as a grey solid (1.5 g) which was used without further purification. ¹H NMR (400 MHz, MeOH-d₄) δ: 8.34 (s, 1H), 8.09 (s, 1H), 7.95 (d, 1H), 7.66 (d, 1H), 7.00 (s, 1H), 3.99 (s, 3H).

### Preparation 153

### 5-chloro-7-(1-methyl-1H-pyrazol-4-yl)imidazo[1,2-c]pyrimidine

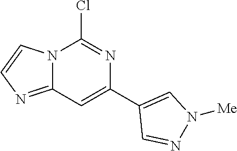 [SMILES: Cn1cc(-c2cc3nccn3c(Cl)n2)cn1]

TEA (752 mg, 7.43 mmol) was added to a solution of 7-(1-methyl-1H-pyrazol-4-yl)imidazo[1,2-c]pyrimidin-5-ol (Preparation 152, 800 mg, 3.72 mmol) in POCl₃ (3.0 mL) at 25° C. The resulting mixture was stirred at 100° C. for 16 h. The reaction was evaporated to dryness in vacuo and the residue purified by prep-HPLC-13 (gradient: 0-25%) to give 5-chloro-7-(1-methyl-1H-pyrazol-4-yl)imidazo[1,2-c]pyrimidine as a brown solid (60 mg, 7%). ¹H NMR (400 MHz, CDCl₃) δ: 8.03-8.01 (m, 2H), 7.81-7.72 (m, 3H), 3.99 (s, 3H).

### Preparation 154

### 6-bromo-4-methoxypyrazolo[1,5-a]pyridine

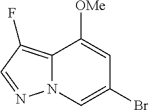 [SMILES: COc1cc(Br)cn2ncc(F)c12]

To a solution of Selectfluor™ (5.15 g, 14.5 mmol) in MeCN (50 mL) was added 6-bromo-4-methoxypyrazolo[1,5-a]pyridine (3 g, 13.2 mmol) at 0° C. and the reaction mixture stirred at 0° C. for 12 h. The reaction mixture was concentrated and treated with water (100 mL), extracted with DCM (2×100 mL). The combined organics were washed with brine (100 mL), dried (Na₂SO₄) and evaporated to dryness in vacuo. The residue was purified by column chromatography (SiO₂, 0-30%, EtOAc/PE) and prep-HPLC-7 (gradient: 37-60%) followed by lyophilization to give 6-bromo-4-methoxypyrazolo[1,5-a]pyridine as an off-white solid (205 mg, 6.3%). ¹H NMR (400 MHz, DMSO-d₆) δ: 8.56 (d, 1H), 8.04 (d, 1H), 6.77 (s, 1H), 3.96 (s, 3H).

### Preparation 155

### 6-bromo-3-fluoropyrazolo[1,5-a]pyridin-4-ol

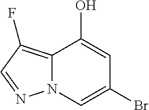 [SMILES: Oc1cc(Br)cn2ncc(F)c12]

A mixture of 6-bromo-4-methoxypyrazolo[1,5-a]pyridine (Preparation 154, 200 mg, 0.816 mmol) in aqueous HBr (165 mg, 0.816 mmol, 20 mL, 40% purity) was stirred at 120° C. for 12 h. The mixture was concentrated and the residue purified by preparative HPLC-7 (gradient: 8-48%) followed by lyophilization to afford 6-bromo-3-fluoropyrazolo[1,5-a]pyridin-4-ol as an off-white solid (150 mg, 80%). ¹H NMR (400 MHz, DMSO-d₆) δ: 11.32 (br, s, 1H), 8.40 (s, 1H), 7.96 (d, 1H), 6.44 (s, 1H).

### Preparation 156

### 6-(1-(difluoromethyl)-1H-pyrazol-4-yl)-3-fluoropyrazolo[1,5-a]pyridin-4-ol

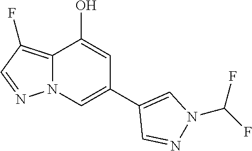 [SMILES: Oc1cc(-c2cnn(C(F)F)c2)cn2ncc(F)c12]

To a solution of 6-bromo-3-fluoropyrazolo[1,5-a]pyridin-4-ol (Preparation 155, 100 mg, 0.433 mmol) in dioxane (5 mL) and H₂O (0.5 mL) was added 1-(difluoromethyl)-4-(4,4,5,5-tetramethyl-1,3,2-dioxaborolan-2-yl)-1H-pyrazole (158 mg, 1.08 mmol), PEPPSi-IPr (29.5 mg, 0.087 mmol) and K₂CO₃ (179.5 mg, 1.30 mmol) and the mixture stirred at 100° C. for 16 h under N₂. Additional 1-(difluoromethyl)-4-(4,4,5,5-tetramethyl-1,3,2-dioxaborolan-2-yl)-1H-pyrazole (105.6 mg, 1.08 mmol) and PEPPSi-IPr (29.5 mg, 0.087 mmol) was added and the mixture was stirred at 100° C. for 2 h under N₂. The reaction mixture was diluted with water (15 mL), extracted with EtOAc (3×20 mL). The combined organics were washed with brine (60 mL), dried (Na₂SO₄) and evaporated to dryness in vacuo. The residue was purified by column chromatography (SiO2; 0-2-% EtOAc/PE) to give 6-(1-(difluoromethyl)-1H-pyrazol-4-yl)-3-fluoropyrazolo[1,5-a]pyridin-4-ol as an orange solid (50.4 mg, crude). LCMS m/z=269.0 [M+H]⁺.

### Preparation 157

### 6-(1-(difluoromethyl)-1H-pyrazol-4-yl)-3-fluoropyrazolo[1,5-a]pyridin-4-yl trifluoromethanesulfonate

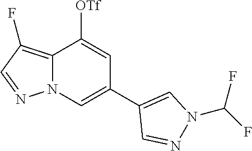 [SMILES: O=S(=O)(Oc1cc(-c2cnn(C(F)F)c2)cn2ncc(F)c12)C(F)(F)F]

To a solution of 6-(1-(difluoromethyl)-1H-pyrazol-4-yl)-3-fluoropyrazolo[1,5-a]pyridin-4-ol (Preparation 156, 40 mg, 0.149 mmol) in THF (3 mL) was added DIPEA (96.4 mg, 0.746 mmol) and Tf₂NPh (79.9 mg, 0.224 mmol) and the mixture stirred at 25° C. for 1 h. The mixture was diluted with water (5 mL) and extracted with EtOAc (3×10 mL). The combined organics were washed with brine (20 mL), dried (Na₂SO₄) and evaporated to dryness in vacuo. The residue was purified by column chromatography (SiO₂; 0-25% EtOAc/PE) to give 6-(1-(difluoromethyl)-1H-pyrazol-4-yl)-3-fluoropyrazolo[1,5-a]pyridin-4-yl trifluoromethanesulfonate as a white solid (33.8 mg) which was used without further purification. ¹H NMR (500 MHz, CDCl₃) δ: 8.47 (s, 1H), 8.09 (s, 1H), 7.91 (d, 1H), 7.26 (t, 1H), 7.18 (s, 1H).

### Preparation 158

### 1-amino-3-bromo-5-methoxypyridin-1-ium 2,4-dinitrophenolate

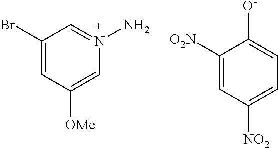

To a solution of 3-bromo-5-methoxypyridine (50.0 g, 266 mmol) in MeCN (500 mL) was added O-(2,4-dinitrophenyl)hydroxylamine (58.3 g, 293 mmol) with stirring at 20 to 25° C. and the resulting mixture heated to 40° C. for 16 h. The mixture was allowed to cool to 20° C. and the solids collected by filtration and the filter cake washed with MTBE (100 mL). The solids were dried under vacuum with no heat for 1 h to provide 1-amino-3-bromo-5-methoxypyridin-1-ium 2,4-dinitrophenolate as a yellow solid (75 g, 73%). ¹H NMR (400 MHz, MeOH-d₄) δ: 8.75 (d, 1H), 8.60 (t, 1H), 8.52-8.49 (m, 1H), 8.19-8.18 (m, 1H), 7.99 (dd, 1H), 6.65 (d, 1H), 4.03 (s, 3H).

### Preparation 159

### ethyl 6-bromo-4-methoxypyrazolo[1,5-a]pyridine-3-carboxylate and ethyl 4-bromo-6-methoxypyrazolo[1,5-a]pyridine-3-carboxylate

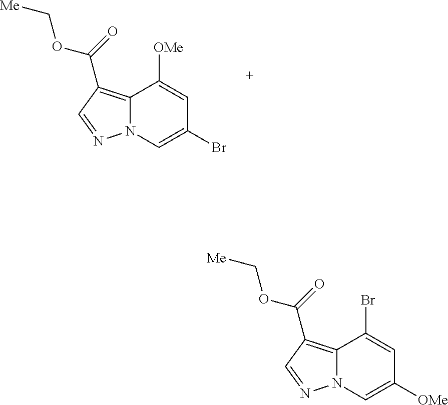 [SMILES: CCOC(=O)c1cnn2cc(Br)cc(OC)c12 ; CCOC(=O)c1cnn2cc(OC)cc(Br)c12]

To a solution of 1-amino-3-bromo-5-methoxypyridin-1-ium 2,4-dinitrophenolate (Preparation 158, 90 g, 232 mmol) in DMF (1500 mL) was added K₂CO₃ (64.3 g, 465 mmol) followed by ethyl prop-2-ynoate (45.61 g, 465 mmol) and the mixture was stirred at 20° C. for 48 h. The reaction mixture was diluted with H₂O (800 mL) and extracted with EtOAc (3×500 mL). The combined organics were dried (Na₂SO₄) and evaporated to dryness in vacuo. The residue was purified by column chromatography (SiO₂, 3-50% EtOAc/PE) to give a mixture of ethyl 6-bromo-4-methoxypyrazolo[1,5-a]pyridine-3-carboxylate and ethyl 4-bromo-6-methoxypyrazolo[1,5-a]pyridine-3-carboxylate as a yellow solid (80 g, 57.5%) which was used without further separation. ¹H NMR (400 MHz, CDCl₃) δ: 8.31 (s, 2H), 6.73 (s, 1H), 4.29 (q, J=6.8 Hz, 2H), 4.01 (s, 3H), 1.27-1.37 (m, 3H).

### Preparation 160

### 6-bromo-4-methoxypyrazolo[1,5-a]pyridine and 4-bromo-6-methoxypyrazolo[1,5-a]pyridine

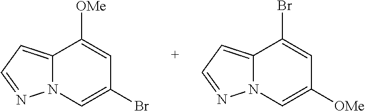 [SMILES: COc1cc(Br)cn2nccc12 ; COc1cc(Br)c2ccnn2c1]

Sulfuric acid (276 g, 2.81 mol, 150 mL) was added to a solution of a mixture of ethyl 6-bromo-4-methoxypyrazolo[1,5-a]pyridine-3-carboxylate and ethyl 4-bromo-6-methoxypyrazolo[1,5-a]pyridine-3-carboxylate (Preparation 159, 46.6 g, 155 mmol, ~3:1 mixture) in water (300 mL) and the reaction mixture stirred at 90° C. for 16 h under N₂. The reaction mixture was quenched with NaOH (2N) until pH to 8 and extracted with EtOAc (3×350 mL) and the combined organics dried (Na₂SO₄) and evaporated to dryness in vacuo. The residue was purified by column chromatography (SiO₂, 5-50% EtOAc/PE) to give an approximately 4:1 mixture of 6-bromo-4-methoxypyrazolo[1,5-a]pyridine and 4-bromo-6-methoxypyrazolo[1,5-a]pyridine as a yellow solid (20 g, 75%). NMR of the major component (6-bromo-4-methoxypyrazolo[1,5-a]pyridine) ¹H NMR (CDCl₃, 400 MHz) δ: 8.28 (s, 1H), 7.85 (d, 1H), 6.63 (d, 1H), 6.46 (d, 1H), 3.96 (s, 3H).

### Preparation 161

### 4-methoxy-6-(1-methyl-1H-pyrazol-4-yl)pyrazolo[1,5-a]pyridine

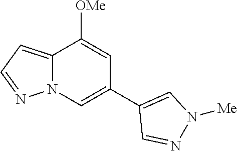 [SMILES: COc1cc(-c2cnn(C)c2)cn2nccc12]

To a solution of an approximately 4:1 mixture of 6-bromo-4-methoxypyrazolo[1,5-a]pyridine and 4-bromo-6-methoxypyrazolo[1,5-a]pyridine (Preparation 160, 60 g, 264 mmol) and 1-methyl-4-(4,4,5,5-tetramethyl-1,3,2-dioxaborolan-2-yl)-1H-pyrazole (66 g, 317 mmol) in dioxane (500 mL) and water (50 mL) was added Pd(dppf)Cl₂·DCM (10.8 g, 13.2 mmol) and K₂CO₃ (73 g, 528 mmol) and the mixture stirred at 90° C. for 2 h under N₂. The reaction mixture was diluted with H₂O (250 mL) and extracted with EtOAc (3×200 mL) and the combined organics dried (Na₂SO₄) and evaporated to dryness in vacuo. The residue was purified by column chromatography (SiO₂, 5-50% EtOAc/PE) to give 4-methoxy-6-(1-methyl-1H-pyrazol-4-yl)pyrazolo[1,5-a]pyridine as a white solid (46 g, 76% yield). ¹H NMR (400 MHz, CDCl₃) δ: 8.27 (s, 1H), 7.86 (d, 1H), 7.74 (s, 1H), 7.61 (s, 1H), 6.62 (s, 1H), 6.47 (s, 1H), 4.00 (s, 3H), 3.97 (s, 3H).

### Preparation 162

### 6-(1-methyl-1H-pyrazol-4-yl)pyrazolo[1,5-a]pyridin-4-ol

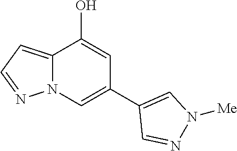 [SMILES: Cn1cc(-c2cc(O)c3ccnn3c2)cn1]

To a solution of 4-methoxy-6-(1-methyl-1H-pyrazol-4-yl)pyrazolo[1,5-a]pyridine (Preparation 161, 46 g, 202 mmol) in aq. HBr (600 mL, 40% purity) was stirred at 120° C. for 72 h. The reaction mixture was concentrated to give the residue treated with sat.NaHCO3 until pH to 8 and extracted with DCM (3×250 mL). The combined organics was dried (Na₂SO₄) and evaporated to dryness in vacuo. The residue was purified by column chromatography (SiO₂, 5-10% MeOH/DCM) to give 6-(1-methyl-1H-pyrazol-4-yl)pyrazolo[1,5-a]pyridin-4-ol as a grey solid (40 g, 93%). LCMS m/z=215.1 [M+H]⁺.

### Preparation 163

### 6-(1-methyl-1H-pyrazol-4-yl)pyrazolo[1,5-a]pyridin-4-yl trifluoromethanesulfonate

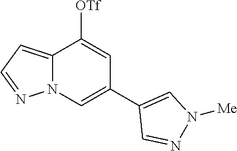 [SMILES: Cn1cc(-c2cc(OS(=O)(=O)C(F)(F)F)c3ccnn3c2)cn1]

6-(1-methyl-1H-pyrazol-4-yl)pyrazolo[1,5-a]pyridin-4-yl trifluoromethanesulfonate was prepared as a white solid (22 g, 68%) from 6-(1-methyl-1H-pyrazol-4-yl)pyrazolo[1,5-a]pyridin-4-ol (Preparation 162) using an analogous method to that described for Preparation XX. ¹H NMR (400 MHz, CDCl3) δ: 8.60 (s, 1H), 8.00 (d, 1H), 7.74 (s, 1H), 7.64 (s, 1H), 7.23 (s, 1H), 6.68 (d, 1H), 3.99 (s, 3H).

### Preparation 164

### 1-methyl-4-((trimethylsilyl)ethynyl)-1H-pyrazole

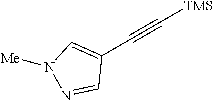 [SMILES: Cn1cc(C#C[Si](C)(C)C)cn1]

To a solution of 4-iodo-1-methyl-1H-pyrazole (4.0 g, 19.2 mmol) and ethynyltrimethylsilane (2.83 g, 28.9 mmol, 4.08 mL) in TEA (40 mL) was added Pd(PPh₃)₂Cl₂ (1.35 g, 1.92 mmol) and CuI (366 mg, 1.92 mmol) and the mixture stirred at 100° C. for 4 h under N₂. The mixture was quenched with water (50 mL) and extracted with DCM (3×50 mL). The combined organics were washed with brine (50 mL), dried (Na₂SO₄) and evaporated to dryness in vacuo and the residue purified using column chromatography (SiO₂, 0-33% EtOAc/PE) to yield 1-methyl-4-((trimethylsilyl)ethynyl)-1H-pyrazole as a brown solid (2.3 g, 66%). ¹H NMR (500 MHz, CDCl₃) δ: 7.57 (s, 1H), 7.49 (s, 1H), 3.87 (s, 3H), 0.22 (s, 9H).

### Preparation 165

### 4-(1-methyl-1H-pyrazol-4-yl)but-3-yn-2-one

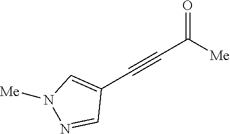 [SMILES: CC(=O)C#Cc1cnn(C)c1]

To a solution of 1-methyl-4-((trimethylsilyl)ethynyl)-1H-pyrazole (Preparation 164, 2.3 g, 12.6 mmol) and acetyl chloride (1.2 g, 15.1 mmol) in DCM (200 mL) was added AlCl₃ (8.4 g, 63.09 mmol). The mixture was stirred at 0° C. for 15 min. The mixture was quenched with water (50 mL) and extracted with DCM (3×30 mL). The combined organic layers were washed with brine (50 mL), dried (Na₂SO₄) and concentrated. The residue was purified by column chromatography (SiO₂, 0-50% EtOAc/PE) to afford 4-(1-methyl-1H-pyrazol-4-yl)but-3-yn-2-one as yellow solid (1.2 g, 61.5%).

¹H NMR (400 MHz, CDCl₃) δ: 7.68 (s, 1H), 7.64 (s, 1H), 3.91 (s, 3H), 2.39 (s, 3H).

### Preparation 166

### tert-butyl ((mesitylsulfonyl)oxy)carbamate

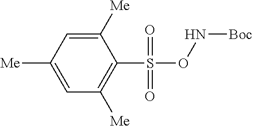 [SMILES: Cc1cc(C)c(S(=O)(=O)ONC(=O)OC(C)(C)C)c(C)c1]

A solution of 2,4,6-trimethylbenzenesulfonyl chloride (10.0 g, 45.7 mmol) and tert-butyl hydroxycarbamate (6.7 g, 50.3 mmol) in THE (160 mL) was stirred at 0° C. TEA (5.6 g, 54.0 mmol, 7.7 mL) was added slowly over 2 min and the mixture was stirred at 0° C. for 1 h. The mixture was evaporated to dryness and the residue treated with water (100 mL) and extracted with DCM (3×50 mL). The combined organics were washed with brine (100 mL), dried (Na₂SO₄) and concentrated under reduced pressure. The residue was purified by column chromatography (SiO₂, 0-10% EtOAc/PE) to give tert-butyl ((mesitylsulfonyl)oxy)carbamate as a white solid (8.5 g, 58.6%). ¹H NMR (400 MHz, DMSO-d₆) δ: 11.12 (s, 1H), 7.10 (s, 2H), 2.54 (s, 6H), 2.26 (s, 3H), 1.21 (s, 9H).

### Preparation 167

### O-(mesitylsulfonyl)hydroxylamine

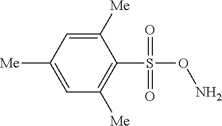 [SMILES: Cc1cc(C)c(S(=O)(=O)ON)c(C)c1]

To a solution of tert-butyl ((mesitylsulfonyl)oxy)carbamate (Preparation 166, 8.5 g, 26.79 mmol) was added TFA (20 mL) at 0° C. and the resulting mixture stirred at 20° C. for 3 h. The reaction mixture was diluted with ice water (40 mL) and stirred at 20° C. for 30 mins. The solids were collected by filtration to afford to 0-(mesitylsulfonyl)hydroxylamine as a white solid (5.0 g, 87%).

¹H NMR (500 MHz, DMSO-d₆) δ: 6.77 (s, 2H), 2.51 (s, 6H), 2.18 (s, 3H).

### Preparation 168

### 1-amino-3-methoxypyridin-1-ium 2,4,6-trimethylbenzenesulfonate

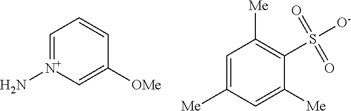 [SMILES: COc1ccc[n+](N)c1 ; Cc1cc(C)c(S(=O)(=O)[O-])c(C)c1]

To a solution of O-(mesitylsulfonyl)hydroxylamine (Preparation 1675.0 g, 23.2 mmol) in DCM (70 mL) was added 3-methoxypyridine (2.8 g, 25.6 mmol) and the mixture stirred at 25° C. for 16 h. The solvent was concentrated to give 1-amino-3-methoxypyridin-1-ium 2,4,6-trimethylbenzenesulfonate as a yellow solid (7.0 g, 93%) which was used without further purification. ¹H NMR (400 MHz, DMSO-d₆) δ: 8.57 (s, 1H), 8.49 (s, 2H), 8.38-8.40 (m, 1H), 7.87-7.90 (m, 2H), 6.73 (s, 2H), 3.94 (s, 3H), 2.47 (s, 6H), 2.15 (s, 3H)

### Preparation 169

### 1-(4-methoxy-2-(1-methyl-1H-pyrazol-4-yl)pyrazolo[1,5-a]pyridin-3-yl)ethan-1-one

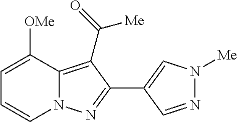 [SMILES: COc1cccn2nc(-c3cnn(C)c3)c(C(C)=O)c12]

To a solution of 4-(1-methyl-1H-pyrazol-4-yl)but-3-yn-2-one (Preparation 165, 1.2 g, 7.76 mmol) and 1-amino-3-methoxypyridin-1-ium 2,4,6-trimethylbenzenesulfonate (Preparation XX, 1.5 g, 11.6 mmol) in EtOH (60 mL) was added K₂CO₃ (3.2 g, 23.3 mmol) and the mixture stirred at 25° C. for 14 h. The mixture was quenched with water (50 mL) and extracted with EtOAc (3×30 mL). The combined organics were washed with brine (50 mL), dried (Na₂SO₄) and evaporated to dryness in vacuo. The residue was purified by column chromatography (SiO2, 0-25% EtOAc/PE) to give 1-(4-methoxy-2-(1-methyl-1H-pyrazol-4-yl)pyrazolo[1,5-a]pyridin-3-yl)ethan-1-one as a yellow solid (880 mg, 42%). ¹H NMR (400 MHz, CDCl₃) δ: 8.13 (d, 1H), 8.08 (s, 1H), 8.01 (s, 1H), 6.76-6.81 (m, 1H), 6.59 (d, 1H), 3.98 (s, 3H), 3.94 (s, 3H), 2.59 (s, 3H).

### Preparation 170

### 1-(4-hydroxy-2-(1-methyl-1H-pyrazol-4-yl)pyrazolo[1,5-a]pyridin-3-yl)ethan-1-one

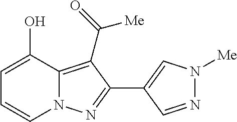 [SMILES: CC(=O)c1c(-c2cnn(C)c2)nn2cccc(O)c12]

To a solution of 1-(4-methoxy-2-(1-methyl-1H-pyrazol-4-yl)pyrazolo[1,5-a]pyridin-3-yl)ethan-1-one (Preparation 169, 800 mg, 2.96 mmol) in DCM (10 mL) was added BBr₃ (1.1 g, 4.44 mmol) at 0° C. under N₂ and then stirred at 25° C. for 12 h. The mixture was quenched with water (20 mL), neutralized with K₂CO₃ and extracted with DCM (3×20 mL). The combined organics were washed with brine (50 mL), dried (Na₂SO₄) and evaporated to dryness in vacuo. The residue was purified by column chromatography (SiO₂, 0-9% MeOH/DCM) to afford 1-(4-hydroxy-2-(1-methyl-1H-pyrazol-4-yl)pyrazolo[1,5-a]pyridin-3-yl)ethan-1-one (334 mg, 44.04% yield) as yellow solid. ¹H NMR (400 MHz, CDCl₃) δ: 8.03-8.06 (m, 1H), 7.70 (s, 1H), 7.66 (s, 1H), 6.95-6.99 (m, 1H), 6.85-6.88 (m, 1H), 4.02 (s, 3H), 2.35 (s, 3H).

### Preparation 171

### 2-(1-methyl-1H-pyrazol-4-yl)pyrazolo[1,5-a]pyridin-4-ol

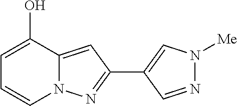 [SMILES: Cn1cc(-c2cc3c(O)cccn3n2)cn1]

To a solution of 1-(4-hydroxy-2-(1-methyl-1H-pyrazol-4-yl)pyrazolo[1,5-a]pyridin-3-yl)ethan-1-one (Preparation 170, 300 mg, 1.17 mmol) in H₂O (0.8 mL) at 25° C. was added H₂SO₄ (20.7 g, 211 mmol) and the mixture stirred at 150° C. for 4 h. The mixture was evaporated to dryness in vacuo and the residue was purified by prep-HPLC-14 (gradient: 15-45%) to give 2-(1-methyl-1H-pyrazol-4-yl)pyrazolo[1,5-a]pyridin-4-ol as a grey-white solid (190 mg, 76%). ¹H NMR (500 MHz, DMSO-d₆) δ: 8.13-8.15 (m, 2H), 7.86 (s, 1H), 6.75 (s, 1H), 6.64-6.67 (m, 1H), 6.44 (d, 1H), 3.90 (s, 3H).

### Preparation 172

### 2-(1-methyl-1H-pyrazol-4-yl)pyrazolo[1,5-a]pyridin-4-yl trifluoromethanesulfonate

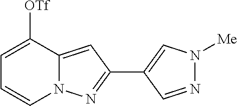 [SMILES: Cn1cc(-c2cc3c(OS(=O)(=O)C(F)(F)F)cccn3n2)cn1]

To a solution of 2-(1-methyl-1H-pyrazol-4-yl)pyrazolo[1,5-a]pyridin-4-ol (Preparation 171, 150 mg, 0.700 mmol) in DCM (10 mL) was added Tf₂O (593 mg, 2.10 mmol) and TEA (213 mg, 2.10 mmol) at 0° C. under N₂ and the resulting mixture stirred at 0° C. for 4 h. The mixture was quenched with water (30 mL) and extracted with DCM (3×10 mL). The combined organics were washed with brine (30 mL), dried (Na₂SO₄) and evaporated to dryness in vacuo. The crude material was purified by column chromatography (SiO₂, 0-50% EtOAc/PE) to give 2-(1-methyl-1H-pyrazol-4-yl)pyrazolo[1,5-a]pyridin-4-yl trifluoromethanesulfonate as a white solid (106 mg, 44%). ¹H NMR (500 MHz, DMSO-d₆) δ: 8.82 (d, 1H), 8.32 (s, 1H), 7.99 (s, 1H), 7.50 (d, 1H), 6.94-6.97 (m, 2H), 3.91 (s, 3H)

### Preparation 173

### 3-(1-methyl-1H-pyrazol-4-yl)-3-oxopropanenitrile

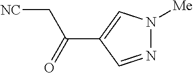 [SMILES: Cn1cc(C(=O)CC#N)cn1]

To a solution of ethyl 1-methyl-1H-pyrazole-4-carboxylate (3.5 g, 22.7 mmol) in THE (20 mL) at −70° C. was added NaHMDS (68.1 mL, 68.1 mmol) under N₂ and the mixture stirred at 25° C. for 3 h. The reaction was quenched with water (50 mL) and extracted with DCM (3×50 mL). The combined organics were washed with brine (50 mL), dried (Na₂SO₄) and evaporated to dryness in vacuo. The residue was purified by column chromatography (SiO2, 0-50% EtOAc/PE) to give 3-(1-methyl-1H-pyrazol-4-yl)-3-oxopropanenitrile as a white solid (2.47 g, 73%). ¹H NMR (400 MHz, CDCl₃) δ: 7.99 (s, 1H), 7.92 (s, 1H), 3.95 (s, 3H), 3.78 (s, 2H).

### Preparation 174

### 1′-methyl-1H,1′H-[3,4′-bipyrazol]-5-amine

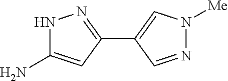 [SMILES: Cn1cc(-c2cc(N)[nH]n2)cn1]

To a solution of 3-(1-methyl-1H-pyrazol-4-yl)-3-oxopropanenitrile (Preparation 173, 1.0 g, 6.70 mmol) in EtOH (20 mL) was added hydrazine hydrate (671 mg, 13.4 mmol) and the mixture stirred at 90° C. for 24 h. The mixture was evaporated to dryness to give 1′-methyl-1H,1′H-[3,4′-bipyrazol]-5-amine as a yellow oil (1.0 g, 92%). ¹H NMR (400 MHz, DMSO-d₆) δ: 7.83 (s, 1H), 7.60 (s, 1H), 5.45 (s, 1H), 4.57 (br s, 2H), 3.79 (s, 3H).

### Preparation 175

### 2-(1-methyl-1H-pyrazol-4-yl)pyrazolo[1,5-a]pyrimidin-7-ol

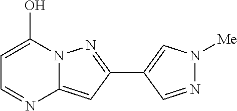 [SMILES: Cn1cc(-c2cc3nccc(O)n3n2)cn1]

To a solution of 1′-methyl-1H,1′H-[3,4′-bipyrazol]-5-amine (Preparation 174, 1.0 g, 6.13 mmol) in EtOH (15 mL) was added sodium (E)-3-ethoxy-3-oxoprop-1-en-1-olate (2.1 g, 18.4 mmol) at 25° C. and then stirred at 100° C. for 6 h. 2N HCl was added to adjust the pH to 5-6. The solids were collected by filtration to afford 2-(1-methyl-1H-pyrazol-4-yl)pyrazolo[1,5-a]pyrimidin-7-ol as a yellow solid (0.75 g, 57%). ¹H NMR (400 MHz, DMSO-d₆) δ: 8.21 (s, 1H), 7.89 (s, 1H), 7.81 (d, 1H), 6.36 (s, 1H), 5.66 (d, 1H), 3.89 (s, 3H).

### Preparation 176

### 7-chloro-2-(1-methyl-1H-pyrazol-4-yl)pyrazolo[1,5-a]pyrimidine

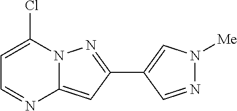 [SMILES: Cn1cc(-c2cc3nccc(Cl)n3n2)cn1]

To a solution of 2-(1-methyl-1H-pyrazol-4-yl)pyrazolo[1,5-a]pyrimidin-7-ol (Preparation 175, 700 mg, 3.25 mmol) in dioxane (10 mL) was added TEA (658 mg, 6.51 mmol) and POCl₃ (1.3 g, 8.13 mmol) at 25° C. and the mixture stirred at 100° C. for 6 h. The mixture neutralized with 2 N NaOH to pH=7, diluted with water (30 mL) and extracted with DCM (3×30 mL). The combined organics were washed with brine (30 mL), dried (Na₂SO₄) and evaporated to dryness in vacuo. The residue was purified by column chromatography (SiO₂, 0-50% EtOAc/PE) to give 7-chloro-2-(1-methyl-1H-pyrazol-4-yl)pyrazolo[1,5-a]pyrimidine as a yellow solid (360 mg, 47%). ¹H NMR (500 MHz, MeOH-d₄) δ ppm: 8.40 (d, 1H), 8.23 (s, 1H), 8.05 (s, 1H), 7.19 (d, 1H), 6.99 (s, 1H), 4.00 (s, 3H).

### Preparation 177

### 5-bromo-3-iodo-1-((2-(trimethylsilyl)ethoxy)methyl)-1H-pyrazolo[3,4-b]pyridine

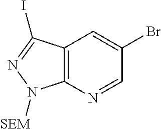 [SMILES: C[Si](C)(C)CCOCn1nc(I)c2cc(Br)cnc21]

To a solution of 5-bromo-3-iodo-1H-pyrazolo[3,4-b]pyridine (300 mg, 0.926 mmol) in DMF (5 mL) was added NaH (37 mg, 0.926 mmol, 60% purity) followed by SEMCl (170 mg mg, 1.02 mmol) and the resulting mixture was stirred at 20° C. for 2 h. The mixture was diluted with H₂O (1 mL) and concentrated to give a residue that was purified by column chromatography (SiO₂, 6-25% EtOAc/PE) to afford 5-bromo-3-iodo-1-((2-(trimethylsilyl)ethoxy)methyl)-1H-pyrazolo[3,4-b]pyridine as a white solid (350 mg, 83%). ¹H NMR (400 MHz, CDCl₃) δ: 8.60 (d, 1H), 7.97 (d, 1H), 5.81 (s, 2H), 3.63-3.67 (m, 2H), 0.90-0.94 (m, 2H), 0.05 (s, 9H).

### Preparation 178

### (3R,4S)-1-(5-bromo-1-((2-(trimethylsilyl)ethoxy)methyl)-1H-pyrazolo[3,4-b]pyridin-3-yl)-3-cyclopropyl-4-methyl-2-oxopyrrolidine-3-carbonitrile

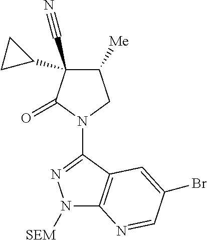 [SMILES: C[C@@H]1CN(c2nn(COCC[Si](C)(C)C)c3ncc(Br)cc23)C(=O)[C@]1(C#N)C1CC1]

To a solution of 5-bromo-3-iodo-1-((2-(trimethylsilyl)ethoxy)methyl)-1H-pyrazolo[3,4-b]pyridine (Preparation 177, 200 mg, 0.440 mmol) and (3R,4S)-3-cyclopropyl-4-methyl-2-oxopyrrolidine-3-carbonitrile (Preparation 21, 72.3 mg, 0.440 mmol) in dioxane (5 mL) was added K₂CO₃ (183 mg, 1.32 mmol), CuI (16.8 mg, 0.088 mmol) and N,N′-dimethylethane-1,2-diamine (15.5 mg, 0.176 mmol) and the reaction mixture stirred at 100° C. under N₂ for 3 h. The mixture was concentrated under reduced pressure and the residue was purified by column chromatography (SiO₂, 5-25% EtOAc/PE) to give (3R,4S)-1-(5-bromo-1-((2-(trimethylsilyl)ethoxy)methyl)-1H-pyrazolo[3,4-b]pyridin-3-yl)-3-cyclopropyl-4-methyl-2-oxopyrrolidine-3-carbonitrile as a colourless gum (130 mg, 60%). ¹H NMR (400 MHz, CDCl₃) δ: 9.06 (d, 1H), 8.55 (d, 1H), 5.75 (s, 2H), 4.21-4.26 (m, 1H), 3.73-3.79 (m, 1H), 3.59-3.63 (m, 2H), 3.10-3.14 (m, 1H), 1.41 (d, 3H), 1.11-1.26 (m, 2H), 0.72-0.85 (m, 5H), 0.04 (s, 9H).

### Preparation 179

### (3R,4S)-3-cyclopropyl-4-methyl-1-(5-(1-methyl-1H-pyrazol-4-yl)-1-((2-(trimethylsilyl)ethoxy)methyl)-1H-pyrazolo[3,4-b]pyridin-3-yl)-2-oxopyrrolidine-3-carbonitrile

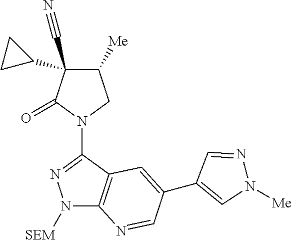 [SMILES: C[C@@H]1CN(c2nn(COCC[Si](C)(C)C)c3ncc(-c4cnn(C)c4)cc23)C(=O)[C@]1(C#N)C1CC1]

To a solution of (3R,4S)-1-(5-bromo-1-((2-(trimethylsilyl)ethoxy)methyl)-1H-pyrazolo[3,4-b]pyridin-3-yl)-3-cyclopropyl-4-methyl-2-oxopyrrolidine-3-carbonitrile (Preparation 178, 130 mg, 0.265 mmol) in dioxane (5 mL) and water (0.5 mL) was added 1-methyl-4-(4,4,5,5-tetramethyl-1,3,2-dioxaborolan-2-yl)-1H-pyrazole (110 mg, 0.530 mmol), KF (46.2 mg, 0.795 mmol) and Pd(dppf)Cl₂ (19.4 mg, 0.027 mmol) under N₂ and the mixture was stirred at 70° C. for 2 h. The reaction mixture was concentrated under reduced pressure and the residue purified by column chromatography (SiO₂, 5-50% EtOAc/PE) to give (3R,4S)-3-cyclopropyl-4-methyl-1-(5-(1-methyl-1H-pyrazol-4-yl)-1-((2-(trimethylsilyl)ethoxy)methyl)-1H-pyrazolo[3,4-b]pyridin-3-yl)-2-oxopyrrolidine-3-carbonitrile as a brown solid (70 mg, 54%). ¹H NMR (500 MHz, CDCl₃) δ: 8.74-8.90 (m, 2H), 7.87-7.90 (m, 2H), 5.80 (s, 2H), 3.65-4.25 (m, 7H), 3.11-3.26 (m, 1H), 1.24-1.27 (m, 4H), 0.81-0.95 (m, 6H), 0.04 (s, 9H).

### Preparation 180

### 3-bromo-1-(1-methyl-1H-pyrazol-4-yl)-1H-pyrazolo[3,4-c]pyridine

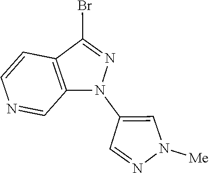 [SMILES: Cn1cc(-n2nc(Br)c3ccncc32)cn1]

To a solution of 3-bromo-1H-pyrazolo[3,4-c]pyridine (200 mg, 1.01 mmol) in DMF (5 mL) was added (1-methyl-1H-pyrazol-4-yl)boronic acid (191 mg, 1.51 mmol), Cu(OAc)₂ (184 mg, 1.01 mmol) and pyridine (400 mg, 5.05 mmol) and the mixture stirred at 50° C. for 40 h. The mixture was purified by prep-HPLC-2 (gradient: 25-55%) to give 3-bromo-1-(1-methyl-1H-pyrazol-4-yl)-1H-pyrazolo[3,4-c]pyridine as a white solid (80 mg, 28%). LCMS m/z=280.0 [M+H]⁺.

### Preparation 181-183

The title compounds were prepared from the appropriate bromide (RBr) and appropriate boronic acid/ester (RBY) using an analogous method to that described for Preparation 180.

| Preparation | |
|---|---|
| No | Name/Structure/RBr/RBY/Data |
| 181 | 3-bromo-1-(1-ethyl-1H-pyrazol-4-yl)-1H-pyrazolo[3,4-c]pyridine |
| | 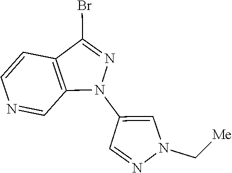 [SMILES: CCn1cc(-n2nc(Br)c3ccncc32)cn1] |
| | RBr: 3-bromo-1H-pyrazolo[3,4-c]pyridine |
| | RBY: 1-ethyl-4-(4,4,5,5-tetramethyl-1,3,2-dioxaborolan-2-yl)-1H-pyrazole |
| | Purification: SiO2, 9-66% EtOAc/PE |
| | Yield: Yellow oil (120 mg, 27%). LCMS m/z = 294.1 [M + H]⁺ |
| 182 | 3-bromo-1-(1-(difluoromethyl)-1H-pyrazol-4-yl)-1H-pyrazolo[3,4- |
| | c]pyridine |
| | 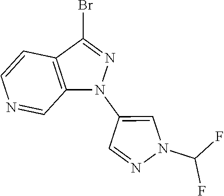 [SMILES: FC(F)n1cc(-n2nc(Br)c3ccncc32)cn1] |
| | RBr: 3-bromo-1H-pyrazolo[3,4-c]pyridine |
| | RBY: 1-(difluoromethyl)-4-(4,4,5,5-tetramethyl-1,3,2-dioxaborolan-2-yl)- |
| | 1H-pyrazole |
| | Purification: Boston Green ODS 150*30 mm*5 um; Condition: water |
| | (0.1% TFA)-ACN; Begin B: 33; End B: 53; Gradient Time (min): 10; |
| | 100% B Hold Time (min): 2; Flow Rate (ml/min): 25; Detection |
| | wavelength: 220 nm) |
| | Yield: Yellow solid (80 mg, 17%). LCMS m/z = 314.1 [M + H]⁺ |
| 183 | 3-bromo-1-(1-(tetrahydro-2H-pyran-2-yl)-1H-pyrazol-4-yl)-1H- |
| | pyrazolo[3,4-c]pyridine |
| | 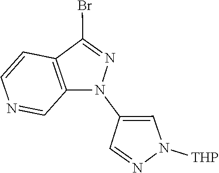 |
| | RBr: 3-bromo-1H-pyrazolo[3,4-c]pyridine |
| | RBY: 1-(tetrahydro-2H-pyran-2-yl)-4-(4,4,5,5-tetramethyl-1,3,2- |
| | dioxaborolan-2-yl)-1H-pyrazole |
| | Purification: Boston Green ODS 150*30 mm*5 um; Condition: water |
| | (0.1% TFA)-ACN; Begin B: 33; End B: 53; Gradient Time (min): 10; |
| | 100% B Hold Time (min): 2; Flow Rate (ml/min): 25; Detection |
| | wavelength: 220 nm) |
| | Yield: Yellow solid (80 mg, 17%). LCMS m/z = 314.1 [M + H]⁺ |

### Preparation 184

### 3-bromo-1-(1-(tetrahydro-2H-pyran-2-yl)-1H-pyrazol-4-yl)-1H-pyrazolo[3,4-c]pyridine

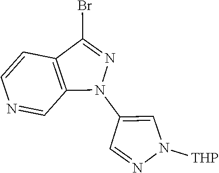

To a solution of 3-bromo-1H-pyrazolo[3,4-c]pyridine (1.0 g, 5.05 mmol), 1-(tetrahydro-2H-pyran-2-yl)-4-(4,4,5,5-tetramethyl-1,3,2-dioxaborolan-2-yl)-1H-pyrazole 1.4 g, 5.05 mmol) in DMSO (10 mL) was added Cu(OAc)₂ (459 mg, 2.52 mmol), 2-(2-pyridyl)pyridine (789 mg, 5.05 mmol) and Cs₂CO₃ (3.29 g, 10.1 mmol) at 25° C. The mixture was stirred at 100° C. for 16 h. The mixture was quenched with water (50 mL) and extracted with EtOAc (3×30 mL). The combined organic layers were washed with H₂O (5×50 ml), dried (Na₂SO₄) and evaporated to dryness in vacuo. The residue was purified by column chromatography (SiO2, 25-66% EtOAc/PE) to give 3-bromo-1-(1-(tetrahydro-2H-pyran-2-yl)-1H-pyrazol-4-yl)-1H-pyrazolo[3,4-c]pyridine as a yellow solid (170 mg, 10%) as. ¹H NMR (500 MHz, CDCl₃) δ: 9.07 (s, 1H), 8.47 (d, 1H), 8.08 (s, 1H), 7.92 (s, 1H), 7.58 (d, 1H), 5.46-5.48 (m, 1H), 4.08-4.12 (m, 1H), 3.73-3.78 (m, 1H), 2.16-2.18 (m, 2H), 2.15-2.16 (m, 1H), 1.66-1.75 (m, 3H).

### Preparation 185

### 3-bromo-1-(1H-pyrazol-4-yl)-1H-pyrazolo[3,4-c]pyridine hydrochloride

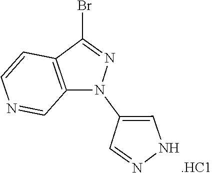

A solution of 3-bromo-1-(1-(tetrahydro-2H-pyran-2-yl)-1H-pyrazol-4-yl)-1H-pyrazolo[3,4-c]pyridine (Preparation 184, 110 mg, 0.316 mmol) in HCl/Dioxane (4 M, 3.93 mL) was stirred at 25° C. for 1 h. The mixture was concentrated under vacuum to afford 3-bromo-1-(1H-pyrazol-4-yl)-1H-pyrazolo[3,4-c]pyridine hydrochloride (70 mg, 74%). LCMS m/z=264.0 [M+H]⁺.

### Preparation 186

### 3-bromo-1-(1-(oxetan-3-yl)-1H-pyrazol-4-yl)-1H-pyrazolo[3,4-c]pyridine

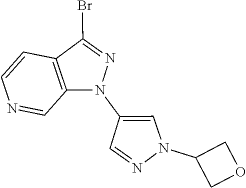 [SMILES: Brc1nn(-c2cnn(C3COC3)c2)c2cnccc12]

A solution of 3-bromo-1-(1H-pyrazol-4-yl)-1H-pyrazolo[3,4-c]pyridine hydrochloride (Preparation 185, 70 mg, 0.265 mmol), 3-iodooxetane (48.8 mg, 0.265 mmol) and Cs₂CO₃ (173 mg, 0.530 mmol) in DMF (4 mL) was stirred at 60° C. for 16 h. The reaction mixture was diluted with EtOAc (20 ml), washed with H₂O (3×20 ml×3) and evaporated to dryness in vacuo. The residue was purified by prep-TLC (50% EtOAc/PE) to give 3-bromo-1-(1-(oxetan-3-yl)-1H-pyrazol-4-yl)-1H-pyrazolo[3,4-c]pyridine is a white solid (50 mg, 59%). ¹H NMR (400 MHz, CDCl₃) δ: 9.10 (s, 1H), 8.49 (d, 1H), 8.08 (s, 1H), 8.01 (s, 1H), 7.61 (d, 1H), 5.51-5.59 (m, 1H), 5.12-5.15 (m, 4H).

### Preparation 187

### 3-bromo-1-(tetrahydro-2H-pyran-2-yl)-1H-pyrazolo[3,4-c]pyridine

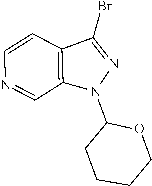 [SMILES: Brc1nn(C2CCCCO2)c2cnccc12]

To a solution of 3,4-dihydro-2H-pyran (19.12 g, 227.25 mmol) in DMF (300 mL) was added 3-bromo-1H-pyrazolo[3,4-c]pyridine (15 g, 75.8 mmol) and toluene-4-sulfonic acid (13.04 g, 75.8 mmol) and the mixture was stirred at 100° C. for 12 h under N₂ atmosphere. The mixture was diluted with water (300 mL) and extracted with EtOAc (3×300 mL). The combined organics were washed with water (3×500 mL), brine (500 mL), dried (Na₂SO₄) and concentrated under reduced pressure. The residue was purified by column chromatography (SiO₂, 9-50% EtOAc/PE) afforded 3-bromo-1-(tetrahydro-2H-pyran-2-yl)-1H-pyrazolo[3,4-c]pyridine as a yellow solid (16.6 g, 78%). ¹H NMR (400 MHz, CDCl₃) δ: 9.16 (s, 1H), 8.41 (d, 1H), 7.53 (dd, 1H), 5.80 (dd, 1H), 4.04-3.97 (m, 1H), 3.83-3.74 (m, 1H), 2.54-2.40 (m, 1H), 2.19-2.11 (m, 2H), 1.80-1.66 (m, 3H).

### Preparation 188

### (3R,4S)-3-cyclopropyl-4-methyl-2-oxo-1-(1-(tetrahydro-2H-pyran-2-yl)-1H-pyrazolo[3,4-c]pyridin-3-yl)pyrrolidine-3-carbonitrile

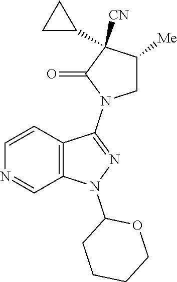 [SMILES: C[C@@H]1CN(c2nn(C3CCCCO3)c3cnccc23)C(=O)[C@]1(C#N)C1CC1]

To a solution of 3-bromo-1-(tetrahydro-2H-pyran-2-yl)-1H-pyrazolo[3,4-c]pyridine (Preparation 187, 16.6 g, 58.84 mmol) in DMSO (300 mL) was added (3R,4S)-3-cyclopropyl-4-methyl-2-oxopyrrolidine-3-carbonitrile (Preparation 21, 9.66 g, 58.8 mmol), copper iodide (4.48 g, 23.53 mmol), pyridine-2-carboxylic acid (2.90 g, 23.53 mmol) and Cs₂CO₃ (38.34 g, 118 mmol) and the mixture was stirred at 110° C. for 12 h under N₂ atmosphere. The mixture was diluted with water (300 mL) and extracted with EtOAc (3×300 mL). The combined organic layers were washed with water (300 mL×3) brine (500 mL), dried (Na₂SO₄) and concentrated under reduced pressure. The residue was purified by column chromatography (SiO₂, 9-50% EtOAc/PE) to give (3R,4S)-3-cyclopropyl-4-methyl-2-oxo-1-(1-(tetrahydro-2H-pyran-2-yl)-1H-pyrazolo[3,4-c]pyridin-3-yl)pyrrolidine-3-carbonitrile as a yellow solid (14 g, 65%). LCMS m/z=366.2 [M+H]⁺.

### Preparation 189

### (3R,4S)-3-cyclopropyl-4-methyl-2-oxo-1-(1H-pyrazolo[3,4-c]pyridin-3-yl)pyrrolidine-3-carbonitrile hydrochloride

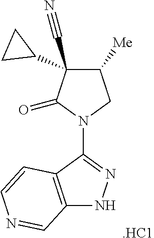

HCl/dioxane (4 M, 200 mL) was added to a solution of (3R,4S)-3-cyclopropyl-4-methyl-2-oxo-1-(1-(tetrahydro-2H-pyran-2-yl)-1H-pyrazolo[3,4-c]pyridin-3-yl)pyrrolidine-3-carbonitrile (Preparation 188, 14 g, 38.31 mmol) in dioxane (50 mL) at 20° C. and the mixture was stirred at 30° C. for 12 h. The mixture was filtered and the filter cake collected and dried under reduced pressure followed by lyophilization to give (3R,4S)-3-cyclopropyl-4-methyl-2-oxo-1-(1H-pyrazolo[3,4-c]pyridin-3-yl)pyrrolidine-3-carbonitrile hydrochloride as a yellow solid (8.3 g, 68%). LCMS m/z=282.1 [M+H]⁺.

### Preparation 190

### 5-bromo-2-(prop-1-yn-1-yl)pyrimidine

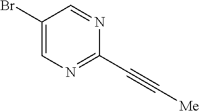

To a solution of 2,5-dibromopyrimidine (5.0 g, 21 mmol) in dioxane (50 mL) was added tributyl(prop-1-yn-1-yl)stannane (7.6 g, 23.1 mmol) and Pd(PPh₃)₂Cl₂ (1.5 g, 2.10 mmol) at 25° C. and the resulting mixture was stirred at 80° C. for 16 h under N₂. The mixture was quenched by 0.5 N NaOH (30 mL) and aq. NaHCO₃ (30 mL) and stirred at 25° C. for 1 h. Then the mixture was diluted with H₂O (50 mL) and extracted with EtOAc (3×80 mL). The combined organics were washed with brine (200 mL), dried (Na₂SO₄) and evaporated to dryness in vacuo. The residue was purified by column chromatography (SiO₂, 5-16% EtOAc/PE) to give 5-bromo-2-(prop-1-yn-1-yl)pyrimidine as a yellow solid (1.9 g, 47%). ¹H NMR (400 MHz, CDCl₃) δ: 8.72 (s, 2H), 2.10 (s, 3H).

### Preparation 191

### 3-bromopyrrolo[1,2-a]pyrimidine

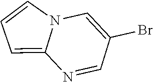

To a solution of 5-bromo-2-(prop-1-yn-1-yl)pyrimidine (Preparation 190, 1.9 g, 10 mmol) in DMA (20 mL) under N₂ was added TEA (7.1 g, 70 mmol) followed by CuI (3.8 g, 20 mmol) and the mixture stirred at 140° C. for 3 h. The reaction was diluted with H₂O (50 mL) and extracted with EtOAc (3×30 mL). The combined organics were washed with brine (60 mL), dried (Na₂SO₄) and evaporated to dryness in vacuo. The residue was purified by column chromatography (SiO₂, 6-16% EtOAc/PE) to give 3-bromopyrrolo[1,2-a]pyrimidine as a yellow (130 mg, 6.6%) as yellow solid. ¹H NMR (400 MHz, MeOD-d₄) δ: 8.81 (d, 1H), 8.01 (d, 1H), 7.38-7.40 (m, 1H), 6.96-6.99 (m, 1H), 6.59 (d, 1H).

### Preparation 192

### 3-bromo-6-iodopyrrolo[1,2-a]pyrimidine

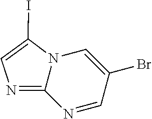 [SMILES: Brc1cnc2ncc(I)n2c1]

To a solution of 3-bromopyrrolo[1,2-a]pyrimidine (Preparation 191, 130 mg, 0.66 mmol) in DCM (10 mL) was added NIS (74.2 mg, 0.33 mol) at 20° C. and the mixture stirred at 20° C. for 1 h. The reaction mixture was concentrated under reduced pressure to give the residue which was purified by column chromatography (SiO₂, 5-15% EtOAc/PE) to give 3-bromo-6-iodopyrrolo[1,2-a]pyrimidine as a yellow solid (136 mg, 64%). ¹H NMR (400 MHz, DMSO-d₆) δ: 8.63 (s, 1H), 8.08 (d, 1H), 7.25 (d, 1H), 6.74 (d, 1H).

### Preparation 193

### (3R,4S)-1-(3-bromopyrrolo[1,2-a]pyrimidin-6-yl)-3-cyclopropyl-4-methyl-2-oxopyrrolidine-3-carbonitrile

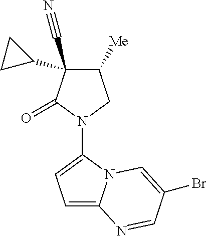 [SMILES: C[C@@H]1CN(c2ccc3ncc(Br)cn23)C(=O)[C@]1(C#N)C1CC1]

To a solution of 3-bromo-6-iodopyrrolo[1,2-a]pyrimidine (Preparation 192, 190 mg, 0.588 mmol) in DMSO (10 mL) was added (3R,4S)-3-cyclopropyl-4-methyl-2-oxopyrrolidine-3-carbonitrile (Preparation 21, 96.6 mg, 0.588 mmol), L-proline (27.1 mg, 0.235 mmol), CuI (22.4 mg, 0.118 mmol) and K₂CO₃ (244 mg, 1.77 mmol) and the resulting mixture stirred at 100° C. under N₂ for 3 h. The reaction was diluted with H₂O (30 mL) and extracted with EtOAc (3×20 mL). The combined organics were washed with brine (50 mL), dried (Na₂SO₄) and evaporated to dryness in vacuo. The reside was purified by column chromatography (SiO₂, 6-25% EtOAc/PE) to give (3R,4S)-1-(3-bromopyrrolo[1,2-a]pyrimidin-6-yl)-3-cyclopropyl-4-methyl-2-oxopyrrolidine-3-carbonitrile as a yellow gum (130 mg, 62%). LCMS m/z=360.9 [M+H]⁺; ¹H NMR (400 MHz, CDCl₃) δ: 8.11 (d, 1H), 7.97 (d, 1H), 6.78 (d, 1H), 6.70 (d, 1H), 3.83-3.88 (m, 1H), 3.57-3.63 (m, 1H), 3.17-3.23 (m, 1H), 1.42 (d, 3H), 1.17-1.21 (m, 1H), 0.79-0.84 (m, 4H).

### Preparation 194

### tert-butyl 5-bromo-3-iodo-1H-pyrrolo[2,3-b]pyridine-1-carboxylate

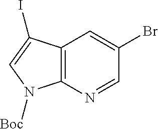 [SMILES: CC(C)(C)OC(=O)n1cc(I)c2cc(Br)cnc21]

Boc₂O (406 mg, 1.86 mmol) was added to a solution of 5-bromo-3-iodo-1H-pyrrolo[2,3-b]pyridine (500 mg, 1.55 mmol) and TEA (313 mg, 3.10 mmol) in DCM (6 mL) and THF (6 mL) under N₂ and the resulting mixture stirred at 20° C. for 16 h. The solvent was removed under reduced pressure and the residue was purified with column chromatography (SiO₂, 0-10% EtOAc/PE) to give tert-butyl 5-bromo-3-iodo-1H-pyrrolo[2,3-b]pyridine-1-carboxylate as a white solid (0.6 g, 92%). ¹H NMR (500 MHz, CDCl₃) δ: 8.53 (s, 1H), 7.85 (d, 1H), 7.80 (s, 1H), 1.66 (s, 9H).

### Preparation 195

### (3R,4S)-1-(5-bromo-1H-pyrrolo[2,3-b]pyridin-3-yl)-3-cyclopropyl-4-methyl-2-oxopyrrolidine-3-carbonitrile

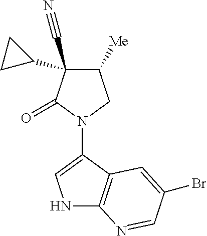 [SMILES: C[C@@H]1CN(c2c[nH]c3ncc(Br)cc23)C(=O)[C@]1(C#N)C1CC1]

To a solution of tert-butyl 5-bromo-3-iodo-1H-pyrrolo[2,3-b]pyridine-1-carboxylate (Preparation 194, 100 mg, 0.236 mmol) and (3R,4S)-3-cyclopropyl-4-methyl-2-oxopyrrolidine-3-carbonitrile (Preparation 21, 46.6 mg, 0.284 mmol) in DMSO (3 mL) was added t-BuOK (53 mg, 0.473 mmol), CuI (4.50 mg, 0.024 mmol) and N,N′-dimethylmethanediamine (1.75 mg, 0.024 mmol) under N₂ and the resulting mixture stirred at 100° C. for 1 h under microwave irradiation. The mixture was purified by prep-HPLC-2 (gradient: 40-70%) to give (3R,4S)-1-(5-bromo-1H-pyrrolo[2,3-b]pyridin-3-yl)-3-cyclopropyl-4-methyl-2-oxopyrrolidine-3-carbonitrile as a white solid. The reaction was repeated 4 times to afford −50 mg of product. LCMS m/z=361.0 [M+H]⁺.

### Preparation 196

### ethyl 4-((1-methyl-1H-pyrazol-4-yl)ethynyl)thiazole-5-carboxylate

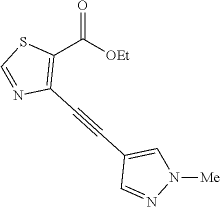 [SMILES: CCOC(=O)c1scnc1C#Cc1cnn(C)c1]

To a solution of 4-ethynyl-1-methyl-1H-pyrazole (607 mg, 5.72 mmol) and ethyl 4-bromothiazole-5-carboxylate (900 mg, 3.81 mmol) in DMF (8 mL) was added CuI (36.3 mg, 0.190 mmol), Pd(PPh₃)₄ (308 mg, 0.267 mmol) and TEA (1.2 g, 11.4 mmol) at 25° C. and the resulting mixture stirred at 70° C. for 3 h under N₂. The mixture was treated with H₂O (20 mL) and extracted with EtOAc (3×20 mL). The combined organics were washed with brine (30 mL), dried (Na₂SO₄) and evaporated to dryness in vacuo. The residue was purified by column chromatography (SiO₂, 5-50% EtOAc/PE) to give ethyl 4-((1-methyl-1H-pyrazol-4-yl)ethynyl)thiazole-5-carboxylate as a yellow solid (830 mg, 83%). LCMS m/z=262.0 [M+H]⁺.

### Preparation 197

### 4-((1-methyl-1H-pyrazol-4-yl)ethynyl)thiazole-5-carboxamide

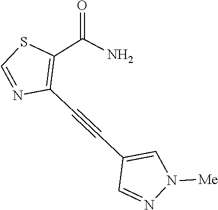 [SMILES: Cn1cc(C#Cc2ncsc2C(N)=O)cn1]

To a solution of ethyl 4-((1-methyl-1H-pyrazol-4-yl)ethynyl)thiazole-5-carboxylate (Preparation 196, 460 mg, 1.76 mmol) in MeOH (2 mL) was added methanolic ammonia solution (7M, 10 mL) at 25° C. and the mixture stirred at 70° C. for 24 h under 15 psi in a 30 mL sealed tube. The mixture was evaporated to dryness in vacuo and the residue purified by column chromatography (SiO₂, 50-100% EtOAc/PE) to give 4-((1-methyl-1H-pyrazol-4-yl)ethynyl)thiazole-5-carboxamide as a pale yellow solid (233 mg, 57%). LCMS m/z=233.0 [M+H]⁺.

### Preparation 198

### 6-(1-methyl-1H-pyrazol-4-yl)thiazolo[5,4-c]pyridin-4(5H)-one

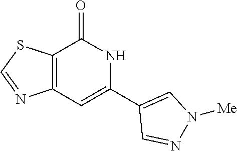 [SMILES: Cn1cc(-c2cc3ncsc3c(=O)[nH]2)cn1]

To a solution of 4-((1-methyl-1H-pyrazol-4-yl)ethynyl)thiazole-5-carboxamide (Preparation 197, 230 mg, 0.990 mmol) in THE (10 mL) was added t-BuONa (133 mg, 1.39 mmol) at 25° C. and the mixture stirred at 70° C. for 4 h. The mixture was concentrated and purified by column chromatography (SiO2, 50-100% EtOAc/PE) to give 6-(1-methyl-1H-pyrazol-4-yl)thiazolo[5,4-c]pyridin-4(5H)-one as a white solid. LCMS m/z=233.1 [M+H]⁺.

### Preparation 199

### 4-chloro-6-(1-methyl-1H-pyrazol-4-yl)thiazolo[5,4-c]pyridine

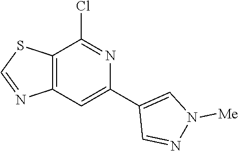 [SMILES: Cn1cc(-c2cc3ncsc3c(Cl)n2)cn1]

A solution of 6-(1-methyl-1H-pyrazol-4-yl)thiazolo[5,4-c]pyridin-4(5H)-one (Preparation 198, 150 mg, 0.646 mmol) in POCl₃ (6 mL) was stirred at 110° C. for 5 h. The reaction mixture was evaporated to dryness in vacuo and the residue treated 5N NaOH to adjust the pH to approx. 7-8. The mixture was diluted with H₂O (10 mL) and extracted with EtOAc (3×15 mL). The combined organics were washed with brine (20 mL), dried (Na₂SO₄) and evaporated to dryness in vacuo to give 4-chloro-6-(1-methyl-1H-pyrazol-4-yl)thiazolo[5,4-c]pyridine as a yellow solid (72 mg, 44%). LCMS m/z=251.0 [M+H]⁺.

### Preparation 200

### (E)-N′-(4,6-dichloropyridin-2-yl)-N,N-dimethylformimidamide

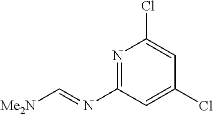 [SMILES: CN(C)/C=N/c1cc(Cl)cc(Cl)n1]

DMF-DMA (4.57 g, 38.3 mmol) was added to a solution of 4,6-dichloropyridin-2-amine (5 g, 30.7 mmol) in EtOH (150 mL) and the reaction mixture heated to 85° C. for 75 mins. The reaction mixture was evaporated to dryness in vacuo to afford (E)-N′-(4,6-dichloropyridin-2-yl)-N,N-dimethylformimidamide as a brown oil (6.6 g, crude) which was used without further purification. ¹H NMR (500 MHz, DMSO-d₆) δ: 8.43 (s, 1H), 7.10 (s, 1H), 6.83 (s, 1H), 3.12 (s, 3H), 3.00 (s, 3H).

### Preparation 201

### (E)-N′-(4,6-dichloropyridin-2-yl)-N-hydroxyformimidamide

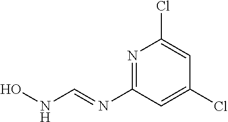 [SMILES: ON/C=N/c1cc(Cl)cc(Cl)n1]

NH₂OH·HCl (3.06 g, 44.02 mmol) was added to a solution of (E)-N′-(4,6-dichloropyridin-2-yl)-N,N-dimethylformimidamide (Preparation 200, 6.0 g, 27.5 mmol) in MeOH (120 mL) under N₂ and the mixture stirred at rt for 1 h. The reaction mixture was evaporated to dryness in vacuo and residue triturated with water. The solids were collected by filtration and washed with water. The product was lyophilized to afford (E)-N′-(4,6-dichloropyridin-2-yl)-N-hydroxyformimidamide (5.1 g, 90%). ¹H NMR (500 MHz, DMSO-d₆) δ: 10.44 (s, 1H), 9.87-9.90 (m, 1H), 7.66-7.71 (m, 1H), 7.11-7.15 (m, 2H).

### Preparation 202

### 5,7-dichloro-[1,2,4]triazolo[1,5-a]pyridine

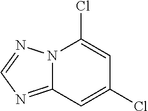 [SMILES: Clc1cc(Cl)n2ncnc2c1]

Eaton's Reagent=Phosphorus pentoxide, 7.7 wt. % in methanesulfonic acid

(E)-N′-(4,6-dichloropyridin-2-yl)-N-hydroxyformimidamide (Preparation 201, 5.0 g, 24.27 mmol) and Eaton's reagent (20 mL) were combined and heated to 105° C. for 20 mins. The mixture was diluted with ice water and basified with solid K₂CO₃ to pH=8 and the resulting solution was extracted with EtOAc (3×50 mL). The combined organics were dried (Na₂SO₄) and evaporated to dryness in vacuo to afford 5,7-dichloro-[1,2,4]triazolo[1,5-a]pyridine as a yellow solid (4.6 g, crude). ¹H NMR (500 MHz, DMSO-d₆) δ: 8.66 (s, 1H), 8.15 (d, 1H), 7.76 (d, 1H).

### Preparation 203

### (3R,4S)-1-(7-chloro-[1,2,4]triazolo[1,5-a]pyridin-5-yl)-3-cyclopropyl-4-methyl-2-oxopyrrolidine-3-carbonitrile

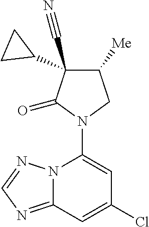 [SMILES: C[C@@H]1CN(c2cc(Cl)cc3ncnn23)C(=O)[C@]1(C#N)C1CC1]

To a solution of (3R,4S)-3-cyclopropyl-4-methyl-2-oxopyrrolidine-3-carbonitrile (Preparation 21, 87.3 mg, 0.532 mmol) and 5,7-dichloro-[1,2,4]triazolo[1,5-a]pyridine (Preparation 202, 100 mg, 0.532 mmol) in DMF (2 mL) was added NaH (25.5 mg, 0.638 mmol, 60% purity) at 0° C. and the mixture stirred at 0° C. for 1 h. The mixture was quenched with water (20 mL) and extracted with EtOAc (2×20 mL). The combined organics were washed with brine (30 mL), dried (Na₂SO₄) and evaporated to dryness in vacuo to afford (3R,4S)-1-(7-chloro-[1,2,4]triazolo[1,5-a]pyridin-5-yl)-3-cyclopropyl-4-methyl-2-oxopyrrolidine-3-carbonitrile as a yellow solid (150 mg, crude) which was used without further purification. LCMS m/z=316.0 [M+H]⁺.

### Preparation 204

### 4-chloro-6-(1-methyl-1H-pyrazol-4-yl)thieno[3,2-d]pyrimidine

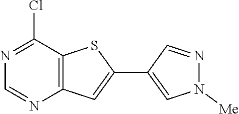 [SMILES: Cn1cc(-c2cc3ncnc(Cl)c3s2)cn1]

To a solution of 6-bromo-4-chlorothieno[3,2-d]pyrimidine (300 mg, 1.20 mmol), 1-methyl-4-(4,4,5,5-tetramethyl-1,3,2-dioxaborolan-2-yl)-1H-pyrazole (625 mg, 3.01 mmol) and Na₂CO₃ (382 mg, 3.61 mmol) in toluene (2 mL) and H₂O (0.2 mL) was added Pd(OAc)₂ (32.4 mg, 0.144 mmol) and PPh₃ (94.6 mg, 0.361 mmol) at 25° C. and the mixture stirred at 110° C. for 2 h. The mixture was quenched with water (50 mL) and extracted with EtOAc (3×30 mL). The combined organics were washed with brine (20 mL), dried (Na₂SO₄) and evaporated to dryness in vacuo. The residue was purified by prep-HPLC-8 (gradient: 37-57%) to give 4-chloro-6-(1-methyl-1H-pyrazol-4-yl)thieno[3,2-d]pyrimidine as a white solid (110 mg, 36%). ¹H NMR (500 MHz, CDCl₃) δ: 8.87 (s, 1H), 8.28 (s, 1H), 8.02 (s, 1H), 7.64 (s, 1H), 3.94 (s, 3H).

### Preparation 205

### 7-bromoimidazo[1,2-a]pyridin-5-amine

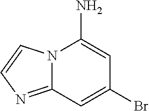 [SMILES: Nc1cc(Br)cc2nccn12]

A solution of 4-bromopyridine-2,6-diamine (1.0 g, 5.32 mmol) and 2-chloroacetaldehyde (1.6 g, 7.98 mmol) in EtOH (10 mL) was stirred at 90° C. for 2 h. The mixture was evaporated to dryness in vacuo and the residue purified by column chromatography (SiO₂, 5-10% MeOH/DCM) to give 7-bromoimidazo[1,2-a]pyridin-5-amine as a white solid (1.1 g, 98%). ¹H NMR (400 MHz, MeOH-d₄) δ: 8.03 (d, 1H), 7.90 (d, 1H), 7.25 (s, 1H), 6.70 (s, 1H)

### Preparation 206

### 7-bromo-5-chloroimidazo[1,2-a]pyridine

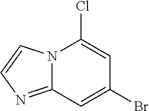 [SMILES: Clc1cc(Br)cc2nccn12]

t-BuONO (486.3 mg, 4.72 mmol, 560.91 uL) was added to CuCl₂ (634 mg, 4.72 mmol) in CH₃CN (20 mL) at 0° C. To this was added 7-bromoimidazo[1,2-a]pyridin-5-amine (Preparation 205, 500 mg, 2.36 mmol) while the internal temperature was allowed to increase to 25° C. during the addition. The resulting mixture was stirred at 25° C. for 3 h. The reaction was diluted with EtOAc (40 mL), followed by saturated NaHCO₃ to pH=8. The resulting suspension was filtered and the filter cake was collected and treated with EtOAc/saturated NaHCO₃ (30 ml/100 ml, ×2) and stirred for 10 mins. The combined filtrates were concentrated and the residue was purified by column chromatography (SiO₂, 0-25% EtOAc/PE) to give 7-bromo-5-chloroimidazo[1,2-a]pyridine as a yellow solid (400 mg, 73%). ¹H NMR (500 MHz, MeOH-d₄) δ: 8.02 (s, 1H), 7.84 (s, 1H), 7.72 (s, 1H), 7.36 (s, 1H).

### Preparation 207

### 5-chloro-7-(1-methyl-1H-pyrazol-4-yl)imidazo[1,2-a]pyridine

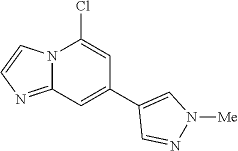 [SMILES: Cn1cc(-c2cc(Cl)n3ccnc3c2)cn1]

To a solution of 7-bromo-5-chloroimidazo[1,2-a]pyridine (Preparation 206, 300 mg, 1.30 mmol), 1-methyl-4-(4,4,5,5-tetramethyl-1,3,2-dioxaborolan-2-yl)-1H-pyrazole (297 mg, 1.43 mmol), CsF (394 mg, 2.59 mmol) in t-amyl alcohol (4 mL) was added Pd(amphos)Cl₂ (92.3 mg, 0.130 mmol) at 25° C. and the resulting mixture stirred at 70° C. for 16 h under N₂. The mixture was diluted with water (50 mL) and extracted with EtOAc (3×20 mL). The combined organics were washed with brine (20 mL), dried (Na₂SO₄) and evaporated to dryness in vacuo. The residue was purified by column chromatography (SiO2, 0-6% MeOH/DCM) to give 5-chloro-7-(1-methyl-1H-pyrazol-4-yl)imidazo[1,2-a]pyridine as a brown solid (140 mg, 46%). ¹H NMR (500 MHz, MeOH-d₄) δ: 8.18 (s, 1H), 8.01 (s, 1H), 7.93 (s, 1H), 7.72 (s, 1H), 7.67 (s, 1H), 7.44 (s, 1H), 3.97 (s, 3H).

### Preparation 208

### ethyl (3S)-4-((tert-butoxycarbonyl)amino)-2-cyano-2-isopropyl-3-methylbutanoate

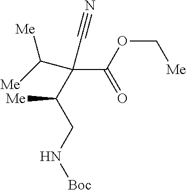 [SMILES: CCOC(=O)C(C#N)(C(C)C)[C@H](C)CNC(=O)OC(C)(C)C]

To a solution of ethyl 2-cyano-3-methylbutanoate (25.0 g, 161.09 mmol) in MeCN (300 mL) were added TBAB (10.39 g, 32.2 mmol) and Cs₂CO₃ (78.73 g, 241.63 mmol) and the solution stirred at 20° C. for 1 h. tert-Butyl (5S)-5-methyl-2,2-dioxo-1,2,3-oxathiazolidine-3-carboxylate (38.22 g, 161.1 mmol) was added in batches at 25° C., then heated to 30° C. and stirred for 16 h. The mixture was filtered and the filtrate concentrated in vacuo. H₂O (300 mL) and aq. HCl (1 mol/L) were added to adjust the pH to 3. The aqueous phase was extracted with EtOAc (200 mL×3) and the combined organic layer was dried to give ethyl (3S)-4-((tert-butoxycarbonyl)amino)-2-cyano-2-isopropyl-3-methylbutanoate (50.32 g, crude) as yellow liquid which was used without further purification.

### Preparation 209

### (3R,4S)-3-isopropyl-4-methyl-2-oxopyrrolidine-3-carbonitrile

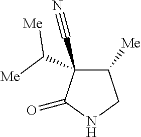 [SMILES: CC(C)[C@@]1(C#N)C(=O)NC[C@H]1C]

To the solution of ethyl (3S)-4-((tert-butoxycarbonyl)amino)-2-cyano-2-isopropyl-3-methylbutanoate (Preparation 208, 50 g, 160.05 mmol) in EtOAc (600 mL), was added toluene-4-sulfonic acid (60.8 g, 320.1 mmol) and the solution heated to 50° C. and stirred for 1 h. The mixture was concentrated in vacuo, the residue suspended in MeCN (700 mL), K₂CO₃ (66.4 g, 480.1 mmol) added and the mixture stirred at 50° C. for 16 h. The reaction was filtered and the filtrate was concentrated under vacuum. The crude product was purified by silica gel column chromatography (eluent: PE/EtOAc=5:1 to 1:1) to give (4S)-3-isopropyl-4-methyl-2-oxopyrrolidine-3-carbonitrile (15 g, 56.4% yield) as a colorless liquid. This was further purified by SFC (ChiralPak IG, 250×50 mm I.D. 10 μm), mobile phase: 15% (0.1% NH₃H₂O in MeOH) at 150 mL/min, column temperature: 40° C., to provide peak 1, 9.0 g, 60%) as a yellow oil, and peak 2, (3R,4S)-3-isopropyl-4-methyl-2-oxopyrrolidine-3-carbonitrile (4.35 g, 29%) as a pale yellow solid. Stereochemistry was determined by 2D nmr. LCMS m/z=167.1 [M+H]⁺; ¹H NMR (400 MHz, CDCl₃) δ ppm: 6.55 (s, 1H), 3.62-3.58 (m, 1H), 3.06-2.97 (m, 2H), 2.21-2.15 (m, 1H), 1.27-1.21 (m, 9H)

### Preparation 210,

### (1s,3s)-3-methoxycyclobutyl methanesulfonate

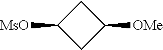 [SMILES: CO[C@H]1C[C@@H](OS(C)(=O)=O)C1]

To a solution of (1s,3s)-3-methoxycyclobutan-1-ol (250 mg, 2.45 mmol) and TEA (743.1 mg, 7.34 mmol) in DCM (10 mL) was added MsCl (500 mg, 4.36 mmol) at 0° C. and the reaction was stirred at 0° C. for 1 h. The reaction was quenched with NH₃·H₂O (1 mL) and MeOH (1 mL). Water (20 mL) was added and the mixture was extracted with DCM (20 mL×3). The collected organics were washed with brine (50 mL), dried over Na₂SO₄, filtered and concentrated to give (1s,3s)-3-methoxycyclobutyl methanesulfonate (200 mg, crude) as yellow oil, which was used for the next step directly without further purification. ¹H NMR (400 MHz, CDCl₃) δ: =4.71-4.65 (m, 1H), 3.61-3.56 (m, 1H), 3.26 (s, 3H), 3.00 (s, 3H), 2.89-2.83 (m, 2H), 2.30-2.24 (m, 2H)

### Preparation 211

### 3-cyano-3-methylcyclobutyl methanesulfonate

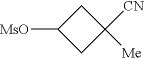 [SMILES: CC1(C#N)CC(OS(C)(=O)=O)C1]

3-Cyano-3-methylcyclobutyl methanesulfonate was obtained as a yellow oil, 170 mg, crude, from 3-hydroxy-1-methylcyclobutane-1-carbonitrile and MsCl following the procedure described in Preparation 210. ¹H NMR (400 MHz, CDCl₃) δ: =5.22-5.04 (m, 1H), 3.11-3.05 (m, 1H), 3.05-3.02 (m, 3H), 2.92-2.86 (m, 1H), 2.66-2.60 (m, 1H), 2.50-2.32 (m, 1H), 1.59 (d, J=18.8 Hz, 3H).

### Preparation 212,

### 2-methyloxetan-3-yl trifluoromethanesulfonate

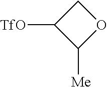 [SMILES: CC1OCC1OS(=O)(=O)C(F)(F)F]

To a solution of 2-methyloxetan-3-ol (200 mg, 2.27 mmol) and DIPEA (1.47 g, 11.35 mmol) in DCM (10 mL) was added Tf₂O (1.60 g, 5.68 mmol) at 0° C. and the reaction was stirred at 0° C. for 1 h. The reaction was quenched with NH₃·H₂O (1 mL) and MeOH (1 mL). Water (20 mL) was added and the mixture was extracted with DCM (20 mL×3). The combined organics were washed with brine (50 mL), dried over Na₂SO₄, filtered and concentrated to give 2-methyloxetan-3-yl trifluoromethanesulfonate (200 mg, crude) as yellow oil, which was used without further purification.

### Preparation 213,

### 3-(4-bromo-1H-pyrazol-1-yl)cyclobutane-1-carbonitrile

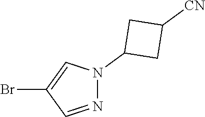 [SMILES: N#CC1CC(n2cc(Br)cn2)C1]

To a solution of 3-cyanocyclobutyl methanesulfonate (0.35 g, 2.00 mmol) in DMF (6 mL) was added Cs₂CO₃ (1.30 g, 4.00 mmol) and 4-bromo-1H-pyrazole (293.60 mg, 2.00 mmol) and the reaction stirred at 90° C. for 2 h. The mixture was concentrated in vacuo and was purified by column chromatography (PE/EtOAc=3:1) to give 3-(4-bromo-1H-pyrazol-1-yl)cyclobutane-1-carbonitrile (500 mg, 88.6% yield) as a white solid. LCMS m/z=228.1 [M+H]⁺

### Preparation 214,

### 4-bromo-1-(2-oxaspiro[3.3]heptan-6-yl)-1H-pyrazole

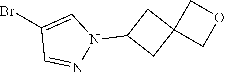 [SMILES: Brc1cnn(C2CC3(COC3)C2)c1]

4-Bromo-1-(2-oxaspiro[3.3]heptan-6-yl)-1H-pyrazole was obtained as a colorless oil, 540 mg, 68.3% yield, from 2-oxaspiro[3.3]heptan-6-yl methanesulfonate and 4-bromo-1H-pyrazole, following a similar procedure to that described in Preparation 213. LCMS m/z=245.1 [M+H]⁺

### Preparation 215,

### 4-bromo-1-((1r,3r)-3-methoxycyclobutyl)-1H-pyrazole

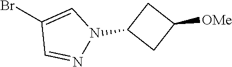 [SMILES: CO[C@H]1C[C@H](n2cc(Br)cn2)C1]

To a solution of 4-bromo-1H-pyrazole (391.45 mg, 2.66 mmol) in DMF (3 mL) was added Cs₂CO₃ (2.17 g, 6.66 mmol) at 20° C. (1s,3s)-3-Methoxycyclobutyl methanesulfonate (Preparation 210, 400 mg, 2.22 mmol) was slowly added and the reaction was stirred at 100° C. for 2 h. The reaction was concentrated in vacuo and water (40 mL) was added. The mixture was extracted with DCM (20 mL×3) and the combined organic extracts dried over Na₂SO₄, filtered and concentrated in vacuo. The crude product was purified by Prep-HPLC-2 (gradient 11-41%) to give 4-bromo-1-((1r,3r)-3-methoxycyclobutyl)-1H-pyrazole (300 mg, 58.5% yield) as colorless oil. LCMS m/z=232.7 [M+H]⁺

### Preparation 216,

### 3-(4-bromo-1H-pyrazol-1-yl)-1-methylcyclobutane-1-carbonitrile

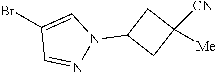 [SMILES: CC1(C#N)CC(n2cc(Br)cn2)C1]

3-(4-Bromo-1H-pyrazol-1-yl)-1-methylcyclobutane-1-carbonitrile was obtained as a yellow solid, 100 mg, 51.5%, from 4-bromo-1H-pyrazole and 3-cyano-3-methylcyclobutyl methanesulfonate (Preparation 211), following a similar procedure to that described in Preparation 215, except the crude product was purified by Prep-HPLC-18 (gradient: 37 to 67%). LCMS m/z=241.8 [M+H]⁺

### Preparation 217,

### 4-bromo-1-(2-methyloxetan-3-yl)-1H-pyrazole

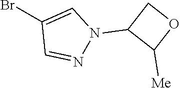 [SMILES: CC1OCC1n1cc(Br)cn1]

To a solution of 4-bromo-1H-pyrazole (133.51 mg, 0.908 mmol) and 2-methyloxetan-3-yl trifluoromethanesulfonate (200 mg, 0.908 mmol) in DMF (10 mL) was added K₂CO₃ (376.65 mg, 2.73 mmol) at 15° C. and the reaction mixture was stirred at 85° C. for 30 h. The reaction was filtered and the filtrate was concentrated in vacuum. The crude product was purified by prep-HPLC-16 (gradient: 27 to 47%) to give 4-bromo-1-(2-methyloxetan-3-yl)-1H-pyrazole (50 mg, 25.4% yield) as a light brown oil. LCMS m/z=217.2 [M+H]⁺

### Preparation 218,

### 4-bromo-3-fluoro-1-(oxetan-3-yl)-1H-pyrazole

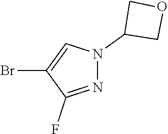 [SMILES: Fc1nn(C2COC2)cc1Br]

A solution of 3-iodooxetane (334.6 mg, 1.82 mmol) in DMF (3 mL), K₂CO₃ (377.02 mg, 2.73 mmol) and 4-bromo-3-fluoro-1H-pyrazole (0.15 g, 0.909 mmol) was stirred at 90° C. for 2 h. The reaction mixture was concentrated in vacuum. The crude product was purified by prep HPLC-18 (gradient: 33 to 53%) to give 4-bromo-3-fluoro-1-(oxetan-3-yl)-1H-pyrazole (130 mg, 64.7% yield) as a white solid. LCMS m/z=220.9 [M+H]⁺

### Preparation 219,

### (1s,3s)-3-(4-bromo-1H-pyrazol-1-yl)-1-methylcyclobutan-1-ol

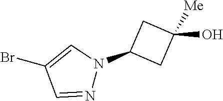

To a solution of 3-(4-bromo-1H-pyrazol-1-yl)cyclobutan-1-one (Preparation 138, 450 mg, 2.09 mmol) in THE (10 mL) at 0° C. was added MeMgBr (3 M, 1.05 mL) dropwise. The reaction was stirred for 16 h, allowing the ice bath to warm to rt. The reaction mixture was quenched by the addition of saturated aqueous NH₄Cl solution (20 mL) at 25° C., then diluted with water (80 mL). The mixture was extracted with DCM (100 mL×3), dried over Na₂SO₄, filtered and concentrated in vacuo. The crude was purified by Prep-HPLC-18 (gradient: 20 to 50%) to give (1s,3s)-3-(4-bromo-1H-pyrazol-1-yl)-1-methylcyclobutan-1-ol (220 mg, 44.6% yield) as yellow oil. LCMS m/z=232.7 [M+H]⁺

### Preparation 220,

### (1s,3s)-3-(4-bromo-1H-pyrazol-1-yl)-1-hydroxycyclobutane-1-carbonitrile

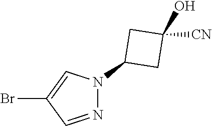 [SMILES: N#C[C@]1(O)C[C@@H](n2cc(Br)cn2)C1]

To a solution of 3-(4-bromo-1H-pyrazol-1-yl)cyclobutan-1-one (Preparation 138, 750 mg, 3.49 mmol) in THE (15 mL) was added ZnI₂ (13.36 g, 41.85 mmol) at 20° C. TMSCN (692.01 mg, 6.98 mmol) was added and the reaction was stirred at 40° C. for 36 h. The mixture was poured into water (50 mL) and extracted with DCM (50 mL×3). The combined organic layers were washed with brine (100 mL), dried over Na₂SO₄, filtered and concentrated under vacuum to give (1s,3s)-3-(4-bromo-1H-pyrazol-1-yl)-1-hydroxycyclobutane-1-carbonitrile (800 mg, crude) as colorless oil. LCMS m/z=242.1 [M+H]⁺

### Preparation 221,

### (1r,3r)-3-(4-bromo-1H-pyrazol-1-yl)-1-fluorocyclobutane-1-carbonitrile

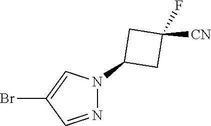 [SMILES: N#C[C@]1(F)C[C@H](n2cc(Br)cn2)C1]

To a solution of (1s,3s)-3-(4-bromo-1H-pyrazol-1-yl)-1-hydroxycyclobutane-1-carbonitrile (Preparation 220, 750 mg, 3.10 mmol) in DCM (20 mL) was added DAST (1.50 g, 9.29 mmol) dropwise at 20° C. The reaction mixture was stirred at 20° C. for 1 h, then quenched with saturated NaHCO₃ solution (20 mL). The mixture was extracted with DCM (20 mL×3), the combined organic layers were washed with brine (100 mL), dried over Na₂SO₄, filtered and concentrated under vacuum to give (1r,3r)-3-(4-bromo-1H-pyrazol-1-yl)-1-fluorocyclobutane-1-carbonitrile (750 mg, crude) as a yellow solid. LCMS m/z=244.1 [M+H]⁺

### Preparation 222,

### O′1,O1-(mesityl-λ3-iodanediyl) 3,3′-dimethyl bis(bicyclo[1.1.1]pentane-1,3-dicarboxylate)

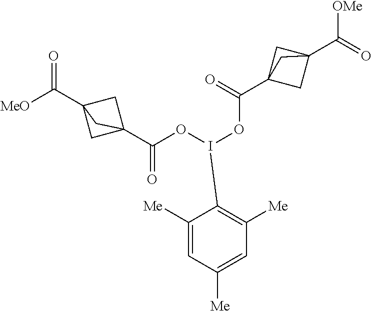 [SMILES: COC(=O)C12CC(C(=O)OI(OC(=O)C34CC(C(=O)OC)(C3)C4)c3c(C)cc(C)cc3C)(C1)C2]

To a solution of 2,4,6-trimethyl(diacetoxyiodo)benzene (1 g, 2.75 mmol) in toluene (50 mL) was added 3-methoxycarbonylbicyclo[1.1.1]pentane-1-carboxylic acid (467.3 mg, 2.75 mmol) at 25° C. and the reaction mixture was stirred at 60° C. for 1 h. After the reaction was completed, the reaction mixture was concentrated in vacuum to give O′1,O1-(mesityl-λ3-iodanediyl) 3,3′-dimethyl bis(bicyclo[1.1.1]pentane-1,3-dicarboxylate) (1.55 g, crude) as a white solid which was used for the next step without further purification. ¹H NMR (400 MHz, CDCl₃) δ=7.03-7.01 (m, 2H), 3.56 (s, 6H), 2.63-2.61 (m, 6H), 2.31-2.28 (m, 6H), 2.12-2.10 (m, 9H).

### Preparation 223,

### methyl 3-(4-bromo-1H-pyrazol-1-yl)bicyclo[1.1.1]pentane-1-carboxylate

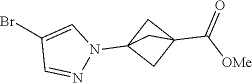 [SMILES: COC(=O)C12CC(n3cc(Br)cn3)(C1)C2]

To a solution of 4-bromo-1H-pyrazole (150.7 mg, 1.03 mmol) in dioxane (5 mL) was added bathophenanthroline (230 mg, 0.692 mmol) and O′1,O1-(mesityl-λ3-iodanediyl) 3,3′-dimethyl bis(bicyclo[1.1.1]pentane-1,3-dicarboxylate) (Preparation 222, 560 mg, 0.958 mmol) at 20° C. Copper (I) thiophene-2-carboxylate (130 mg, 0.682 mmol) was added and the reaction mixture was stirred at 20° C. for 16 h. The reaction mixture was concentrated in vacuo and the residue was poured into water (120 mL). The aqueous solution was extracted with DCM (50 mL×2), the combined organic layers were dried over Na₂SO₄ and concentrated in vacuo. The crude product was purified by prep-TLC (PE/EtOAc=3/1) to give methyl 3-(4-bromo-1H-pyrazol-1-yl)bicyclo[1.1.1]pentane-1-carboxylate (45 mg, crude) as light brown oil. LCMS m/z=271.1 [M+H]⁺

### Preparation 224,

### 3-(4-bromo-1H-pyrazol-1-yl)bicyclo[1.1.1]pentane-1-carboxamide

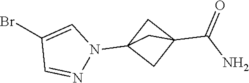 [SMILES: NC(=O)C12CC(n3cc(Br)cn3)(C1)C2]

To a solution of methyl 3-(4-bromo-1H-pyrazol-1-yl)bicyclo[1.1.1]pentane-1-carboxylate (Preparation 223, 45 mg, 0.166 mmol) in MeOH (2 mL) was added into NH₃/MeOH (3 mL, 1M) and the reaction was stirred at 20° C. for 2 h. The reaction mixture was concentrated in vacuum and the crude product was purified by preparative HPLC-16 (gradient: 17 to 37%) to give 3-(4-bromo-1H-pyrazol-1-yl)bicyclo[1.1.1]pentane-1-carboxamide (29.54 mg, 69.5% yield) as a white solid. LCMS m/z=256.2 [M+H]⁺

### Preparation 225,

### 3-(4-bromo-1H-pyrazol-1-yl)bicyclo[1.1.1]pentane-1-carbonitrile

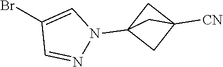 [SMILES: N#CC12CC(n3cc(Br)cn3)(C1)C2]

To a solution of 3-(4-bromo-1H-pyrazol-1-yl)bicyclo[1.1.1]pentane-1-carboxamide (Preparation 224, 40 mg, 0.156 mmol) in MeCN (3 mL) was added SOCl₂ (110.6 mg, 0.937 mmol) at 15° C. The reaction mixture was stirred at 80° C. for 5 h. The reaction mixture was concentrated in vacuo to give 3-(4-bromo-1H-pyrazol-1-yl)bicyclo[1.1.1]pentane-1-carbonitrile (30 mg, crude) as a light brown solid which was used for the next step without further purification. LCMS m/z=238.1 [M+H]⁺

### Preparation 226,

### 1-(bicyclo[1.1.1]pentan-1-yl)-4-bromo-1H-pyrazole

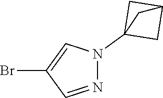 [SMILES: Brc1cnn(C23CC(C2)C3)c1]

A solution of 1-(bicyclo[1.1.1]pentan-1-yl)-1H-pyrazole (0.25 g, 1.86 mmol) in DCM (8 mL) was cooled to 10° C. under Ar(g). Br₂ (893.3 mg, 5.59 mmol) was added and the resulting mixture was stirred at 25° C. for 1.5 h. The solution was quenched with 10% aq. Na₂SO₃ (50 mL), the organic layer was separated, washed with brine (50 mL), dried over Na₂SO₄ and the solvents evaporated to give 1-(bicyclo[1.1.1]pentan-1-yl)-4-bromo-1H-pyrazole (184 mg, crude) as brown oil. ¹H NMR (400 MHz, CDCl₃) δ=7.48 (s, 1H), 7.44 (s, 1H), 2.63 (s, 1H), 2.30-2.28 (m, 6H)

### Preparation 227,

### 5-(4-bromo-1H-pyrazol-1-yl)dihydro-2H-pyran-3(4H)-one

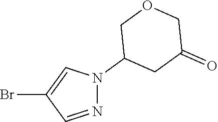 [SMILES: O=C1COCC(n2cc(Br)cn2)C1]

To a solution of 4-bromo-1H-pyrazole (150 mg, 1.02 mmol) in DCM (10 mL) was added ScCl₃ (15.44 mg, 0.102 mmol) at 20° C. 2H-Pyran-3(6H)-one (200.2 mg, 2.04 mmol) was added slowly, then the reaction mixture was stirred at 20° C. for 12 h. The mixture was concentrated in vacuo and the crude product was purified by column chromatography on silica gel (PE/EtOAc=1/0 to 3/1) to give 5-(4-bromo-1H-pyrazol-1-yl)dihydro-2H-pyran-3(4H)-one (220 mg, 79.2% yield) as yellow oil. LCMS m/z=244.9 [M+H]⁺

### Preparation 228,

### 4-bromo-1-(5,5-difluorotetrahydro-2H-pyran-3-yl)-1H-pyrazole

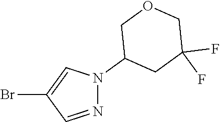 [SMILES: FC1(F)COCC(n2cc(Br)cn2)C1]

To a solution of 5-(4-bromo-1H-pyrazol-1-yl)dihydro-2H-pyran-3(4H)-one (Preparation 227, 200 mg, 0.816 mmol) in DCM (10 mL) was added DAST (394.6 mg, 2.45 mmol) at 0° C. slowly, then the reaction was stirred at 20° C. for 4 h. The mixture was concentrated under vacuum and the crude product was purified by column chromatography on silica gel (PE/EtOAc=1/0 to 3/1) to give 4-bromo-1-(5,5-difluorotetrahydro-2H-pyran-3-yl)-1H-pyrazole (150 mg, 65.4% yield) as yellow oil. LCMS m/z=266.9 [M+H]⁺

### Preparation 229,

### 1-(3,3-difluorocyclobutyl)-4-iodo-1H-pyrazole

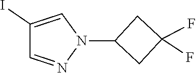 [SMILES: FC1(F)CC(n2cc(I)cn2)C1]

To a solution of 3-(4-iodo-1H-pyrazol-1-yl)cyclobutan-1-one (200 mg, 0.763 mmol) in DCM (10 mL) was added DAST (292.8 mg, 1.82 mmol) at 20° C. and the reaction stirred for 3 h. Sat. aq. NaHCO₃ (15 mL) was added and the mixture was extracted with DCM (3×20 mL). The combined organics were washed with brine (20 mL), then dried over Na₂SO₄, filtered and concentrated under vacuum. The crude was purified by column chromatography on silica gel (PE:EtOAc=3:1) to give 1-(3,3-difluorocyclobutyl)-4-iodo-1H-pyrazole (120 mg, 47.1% yield) as yellow oil. LCMS m/z=285.1 [M+H]⁺

### Preparation 230,

### tert-butyl 6-(4-(4,4,5,5-tetramethyl-1,3,2-dioxaborolan-2-yl)-1H-pyrazol-1-yl)-2-azaspiro[3.3]heptane-2-carboxylate

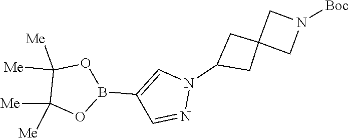 [SMILES: CC(C)(C)OC(=O)N1CC2(CC(n3cc(B4OC(C)(C)C(C)(C)O4)cn3)C2)C1]

N₂ was bubbled through a mixture of tert-butyl 6-(4-bromo-1H-pyrazol-1-yl)-2-azaspiro[3.3]heptane-2-carboxylate (150 mg, 0.438 mmol), (BPin)₂ (222.6 mg, 0.877 mmol), KOAc (129.1 mg, 1.31 mmol) and Pd(dppf)Cl₂ (32.1 mg, 0.0438 mmol) in dioxane (5 mL) and the reaction was stirred at 90° C. for 5 h. The reaction mixture was concentrated in vacuo to give tert-butyl 6-(4-(4,4,5,5-tetramethyl-1,3,2-dioxaborolan-2-yl)-1H-pyrazol-1-yl)-2-azaspiro[3.3]heptane-2-carboxylate (100 mg, crude) as black oil, which was used directly in the next step. LCMS m/z=390.0 [M+H]⁺

### Preparation 231

### 1-(2-oxaspiro[3.3]heptan-6-yl)-4-(4,4,5,5-tetramethyl-1,3,2-dioxaborolan-2-yl)-1H-pyrazole

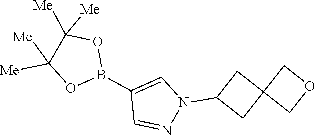 [SMILES: CC1(C)OB(c2cnn(C3CC4(COC4)C3)c2)OC1(C)C]

1-(2-Oxaspiro[3.3]heptan-6-yl)-4-(4,4,5,5-tetramethyl-1,3,2-dioxaborolan-2-yl)-1H-pyrazole was obtained, crude, 250 mg, from 4-bromo-1-(2-oxaspiro[3.3]heptan-6-yl)-1H-pyrazole (Preparation 214), following the method described in Preparation 230. LCMS m/z=291.3 [M+H]⁺

### Preparation 232,

### 3-(4-(4,4,5,5-tetramethyl-1,3,2-dioxaborolan-2-yl)-1H-pyrazol-1-yl)cyclobutane-1-carbonitrile

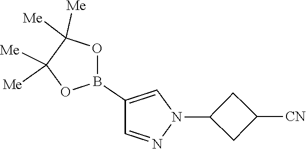 [SMILES: CC1(C)OB(c2cnn(C3CC(C#N)C3)c2)OC1(C)C]

3-(4-(4,4,5,5-Tetramethyl-1,3,2-dioxaborolan-2-yl)-1H-pyrazol-1-yl)cyclobutane-1-carbonitrile was obtained crude as a black oil, from 3-(4-bromo-1H-pyrazol-1-yl)cyclobutane-1-carbonitrile (Preparation 213), following a similar procedure to that described in Preparation 230. LCMS m/z=274.1 [M+H]⁺

### Preparation 233,

### 3-(4-(4,4,5,5-tetramethyl-1,3,2-dioxaborolan-2-yl)-1H-pyrazol-1-yl)bicyclo[1.1.1]pentane-1-carbonitrile

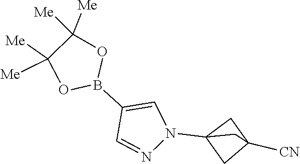 [SMILES: CC1(C)OB(c2cnn(C34CC(C#N)(C3)C4)c2)OC1(C)C]

3-(4-(4,4,5,5-Tetramethyl-1,3,2-dioxaborolan-2-yl)-1H-pyrazol-1-yl)bicyclo[1.1.1]pentane-1-carbonitrile was obtained as a black oil, crude, from 3-(4-bromo-1H-pyrazol-1-yl)bicyclo[1.1.1]pentane-1-carbonitrile (Preparation 225), following a similar procedure to that described in Preparation 230. LCMS m/z=286.3 [M+H]⁺

### Preparation 234,

### 1-(3-methyloxetan-3-yl)-4-(4,4,5,5-tetramethyl-1,3,2-dioxaborolan-2-yl)-1H-pyrazole

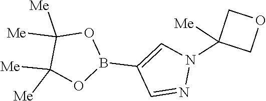 [SMILES: CC1(n2cc(B3OC(C)(C)C(C)(C)O3)cn2)COC1]

1-(3-Methyloxetan-3-yl)-4-(4,4,5,5-tetramethyl-1,3,2-dioxaborolan-2-yl)-1H-pyrazole was obtained as a black oil from 4-bromo-1-(3-methyloxetan-3-yl)-1H-pyrazole, following a similar procedure to that described in Preparation 230. LCMS m/z=264.8 [M+H]⁺

### Preparation 235,

### 1-((1r,3r)-3-methoxycyclobutyl)-4-(4,4,5,5-tetramethyl-1,3,2-dioxaborolan-2-yl)-1H-pyrazole

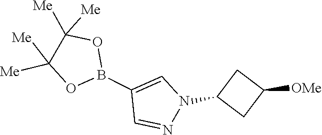 [SMILES: CO[C@H]1C[C@H](n2cc(B3OC(C)(C)C(C)(C)O3)cn2)C1]

To a solution of 4-bromo-1-((1r,3r)-3-methoxycyclobutyl)-1H-pyrazole (Preparation 215, 160 mg, 0.692 mmol) in dioxane (6 mL) was added KOAc (203.9 mg, 2.08 mmol) and (BPin)₂ (263.73 mg, 1.04 mmol) at 20° C. Pd(dppf)Cl₂ (113.1 mg, 0.138 mmol) was added and the reaction was stirred at 90° C. under N₂ for 16 h. The mixture was concentrated under vacuum to give 1-((1r,3r)-3-methoxycyclobutyl)-4-(4,4,5,5-tetramethyl-1,3,2-dioxaborolan-2-yl)-1H-pyrazole (160 mg, crude) as yellow oil, which was used for the next step directly without further purification. LCMS m/z=278.6 [M+H]⁺

### Preparation 236,

### 1-(bicyclo[1.1.1]pentan-1-yl)-4-(4,4,5,5-tetramethyl-1,3,2-dioxaborolan-2-yl)-1H-pyrazole

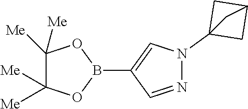 [SMILES: CC1(C)OB(c2cnn(C34CC(C3)C4)c2)OC1(C)C]

To a solution of 1-(bicyclo[1.1.1]pentan-1-yl)-4-bromo-1H-pyrazole (Preparation 226, 174 mg, 0.817 mmol) in dioxane (8 mL) was added KOAc (240.4 mg, 2.45 mmol) and (BPin)₂ (248.8 mg, 0.98 mmol) at 20° C. Pd(dppf)Cl₂ (100.0 mg, 0.122 mmol) was added and the reaction was stirred at 90° C. under N₂ for 16 h. The mixture was concentrated under vacuum and the crude product was purified by column chromatography on silica gel (PE/EtOAc=1/0 to 3/1) to give 1-(bicyclo[1.1.1]pentan-1-yl)-4-(4,4,5,5-tetramethyl-1,3,2-dioxaborolan-2-yl)-1H-pyrazole (69 mg, 32.5% yield) as a yellow solid. ¹H NMR (400 MHz, CDCl₃) δ=7.81 (s, 1H), 7.75 (s, 1H), 2.61 (s, 1H), 2.30 (s, 6H), 1.32 (s, 12H)

### Preparation 237,

### 1-(3,3-difluorocyclobutyl)-4-(4,4,5,5-tetramethyl-1,3,2-dioxaborolan-2-yl)-1H-pyrazole

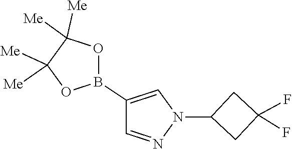 [SMILES: CC1(C)OB(c2cnn(C3CC(F)(F)C3)c2)OC1(C)C]

N₂ was bubbled through a mixture of 1-(3,3-difluorocyclobutyl)-4-iodo-1H-pyrazole (Preparation 229, 120 mg, 0.422 mmol), (BPin)₂ (214.6 mg, 0.845 mmol), KOAc (124.4 mg, 1.27 mmol) and Pd(dppf)Cl₂ (34.5 mg, 0.0423 mmol) in dioxane (6 mL) and the reaction was stirred at 90° C. for 4 h. The reaction mixture was concentrated in vacuum to give crude product, which was purified by column chromatography on silica gel (PE: EtOAc=3:1) to give 1-(3,3-difluorocyclobutyl)-4-(4,4,5,5-tetramethyl-1,3,2-dioxaborolan-2-yl)-1H-pyrazole (80 mg, 53.3% yield) as yellow oil. LCMS m/z=284.9 [M+H]⁺

### Preparation 238,

### 1-(2-methyloxetan-3-yl)-4-(4,4,5,5-tetramethyl-1,3,2-dioxaborolan-2-yl)-1H-pyrazole

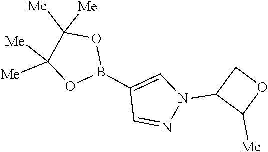 [SMILES: CC1OCC1n1cc(B2OC(C)(C)C(C)(C)O2)cn1]

To a solution of 4-bromo-1-(2-methyloxetan-3-yl)-1H-pyrazole (Preparation 217, 50 mg, 0.230 mmol) and (BPin)₂ (76.04 mg, 0.299 mmol) in dioxane (3 mL) was added KOAc (45.21 mg, 0.460 mmol) and Pd(dppf)Cl₂ (16.85 mg, 0.023 mmol) at 15° C. The reaction mixture was stirred at 90° C. for 6 h then concentrated in vacuo to give 1-(2-methyloxetan-3-yl)-4-(4,4,5,5-tetramethyl-1,3,2-dioxaborolan-2-yl)-1H-pyrazole (60 mg, crude) as a black solid which was used for the next step without further purification. LCMS m/z=265.3 [M+H]⁺

### Preparation 239,

### 1-methyl-3-(4-(4,4,5,5-tetramethyl-1,3,2-dioxaborolan-2-yl)-1H-pyrazol-1-yl)cyclobutane-1-carbonitrile

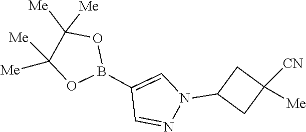 [SMILES: CC1(C#N)CC(n2cc(B3OC(C)(C)C(C)(C)O3)cn2)C1]

To a solution of 3-(4-bromo-1H-pyrazol-1-yl)-1-methylcyclobutane-1-carbonitrile (Preparation 216, 100 mg, 0.416 mmol) in dioxane (8 mL) was added KOAc (122.6 mg, 1.25 mmol) and (BPin)₂ (158.7 mg, 0.625 mmol) at 20° C. Pd(dppf)Cl₂ (68.0 mg, 0.083 mmol) was added and the reaction was stirred at 90° C. under N₂ for 16 h. The mixture was concentrated under vacuum to give 1-methyl-3-(4-(4,4,5,5-tetramethyl-1,3,2-dioxaborolan-2-yl)-1H-pyrazol-1-yl)cyclobutane-1-carbonitrile (110 mg, crude) as yellow oil, which was used for the next step directly without further purification. LCMS m/z=288.2 [M+H]⁺

### Preparation 240,

### (1r,3r)-1-fluoro-3-(4-(4,4,5,5-tetramethyl-1,3,2-dioxaborolan-2-yl)-1H-pyrazol-1-yl)cyclobutane-1-carbonitrile

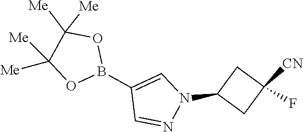 [SMILES: CC1(C)OB(c2cnn([C@H]3C[C@@](F)(C#N)C3)c2)OC1(C)C]

(1r,3r)-1-Fluoro-3-(4-(4,4,5,5-tetramethyl-1,3,2-dioxaborolan-2-yl)-1H-pyrazol-1-yl)cyclobutane-1-carbonitrile was obtained as a yellow oil, 300 mg, crude from (1r,3r)-3-(4-bromo-1H-pyrazol-1-yl)-1-fluorocyclobutane-1-carbonitrile (Preparation 221) following the procedure described in Preparation 239.

### Preparation 241,

### (1s,3s)-1-methyl-3-(4-(4,4,5,5-tetramethyl-1,3,2-dioxaborolan-2-yl)-1H-pyrazol-1-yl)cyclobutan-1-ol

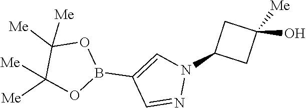 [SMILES: CC1(C)OB(c2cnn([C@H]3C[C@@](C)(O)C3)c2)OC1(C)C]

(1s,3s)-1-Methyl-3-(4-(4,4,5,5-tetramethyl-1,3,2-dioxaborolan-2-yl)-1H-pyrazol-1-yl)cyclobutan-1-ol was obtained as a yellow oil, 120 mg, crude, from (1s,3s)-3-(4-bromo-1H-pyrazol-1-yl)-1-methylcyclobutan-1-ol (Preparation 219), following a similar procedure to that described in Preparation 239. LCMS m/z=279.1 [M+H]⁺

### Preparation 242,

### 1-((4-(4,4,5,5-tetramethyl-1,3,2-dioxaborolan-2-yl)-1H-pyrazol-1-yl)methyl)cyclopropane-1-carbonitrile

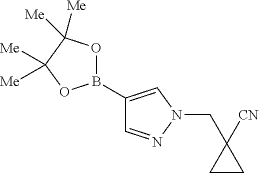 [SMILES: CC1(C)OB(c2cnn(CC3(C#N)CC3)c2)OC1(C)C]

1-((4-(4,4,5,5-Tetramethyl-1,3,2-dioxaborolan-2-yl)-1H-pyrazol-1-yl)methyl)cyclopropane-1-carbonitrile was obtained as a yellow oil, 100 mg, crude, from 1-((4-bromo-1H-pyrazol-1-yl)methyl)cyclopropane-1-carbonitrile, following a similar procedure to that described in Preparation 239. LCMS m/z=274.2 [M+H]⁺

### Preparation 243,

### 1-((3-methyloxetan-3-yl)methyl)-4-(4,4,5,5-tetramethyl-1,3,2-dioxaborolan-2-yl)-1H-pyrazole

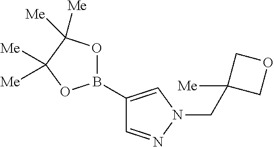 [SMILES: CC1(Cn2cc(B3OC(C)(C)C(C)(C)O3)cn2)COC1]

1-((3-Methyloxetan-3-yl)methyl)-4-(4,4,5,5-tetramethyl-1,3,2-dioxaborolan-2-yl)-1H-pyrazole was obtained as a yellow oil, from 4-bromo-1-((3-methyloxetan-3-yl)methyl)-1H-pyrazole following a similar procedure to that described in Preparation 239. LCMS m/z=279.2 [M+H]⁺

### Preparation 244,

### ethyl 4-((1-(tetrahydro-2H-pyran-2-yl)-1H-pyrazol-4-yl)ethynyl)thiazole-5-carboxylate

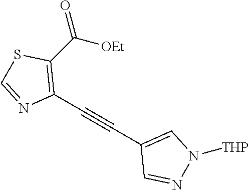

Ethyl 4-((1-(tetrahydro-2H-pyran-2-yl)-1H-pyrazol-4-yl)ethynyl)thiazole-5-carboxylate was obtained as a yellow oil, 4.9 g, 77.6% yield, from 4-ethynyl-1-(tetrahydro-2H-pyran-2-yl)-1H-pyrazole and ethyl 4-bromothiazole-5-carboxylate, following a similar procedure to that described in Preparation 196. LCMS m/z=332.1 [M+H]⁺

### Preparation 245,

### 4-((1-(tetrahydro-2H-pyran-2-yl)-1H-pyrazol-4-yl)ethynyl)thiazole-5-carboxamide

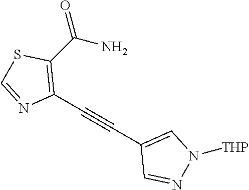

To a solution of ethyl 4-((1-(tetrahydro-2H-pyran-2-yl)-1H-pyrazol-4-yl)ethynyl)thiazole-5-carboxylate (Preparation 244, 4.9 g, 14.79 mmol) in EtOH (60 mL) was added NH₃·H₂O (518.2 mg, 14.79 mmol) and the reaction stirred at 60° C. for 2 h. The mixture was concentrated and the crude purified by silica gel column chromatography (PE/EtOAc=2/1 to 0/1) to give 4-((1-(tetrahydro-2H-pyran-2-yl)-1H-pyrazol-4-yl)ethynyl)thiazole-5-carboxamide (3.66 g, 81.9% yield) as yellow solid. LCMS m/z=303.0 [M+H]⁺

### Preparation 246,

### 6-(1-(tetrahydro-2H-pyran-2-yl)-1H-pyrazol-4-yl)thiazolo[5,4-c]pyridin-4(5H)-one

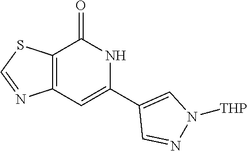

6-(1-(Tetrahydro-2H-pyran-2-yl)-1H-pyrazol-4-yl)thiazolo[5,4-c]pyridin-4(5H)-one was obtained as a yellow solid, 1.85 g, 50.6% yield, from 4-((1-(tetrahydro-2H-pyran-2-yl)-1H-pyrazol-4-yl)ethynyl)thiazole-5-carboxamide (Preparation 245), following the procedure described in Preparation 198. LCMS m/z=302.9 [M+H]⁺

### Preparation 247,

### 4-chloro-6-(1H-pyrazol-4-yl)thiazolo[5,4-c]pyridine

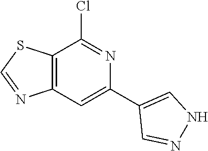 [SMILES: Clc1nc(-c2cn[nH]c2)cc2ncsc12]

4-Chloro-6-(1H-pyrazol-4-yl)thiazolo[5,4-c]pyridine was obtained as a yellow solid, 1.35 g, crude from 6-(1-(tetrahydro-2H-pyran-2-yl)-1H-pyrazol-4-yl)thiazolo[5,4-c]pyridin-4(5H)-one (Preparation 246), following the procedure described in Preparation 199. LCMS m/z=237.0 [M+H]⁺

### Preparation 248,

### (3R,4S)-1-(6-(1H-pyrazol-4-yl)thiazolo[5,4-c]pyridin-4-yl)-3-cyclopropyl-4-methyl-2-oxopyrrolidine-3-carbonitrile

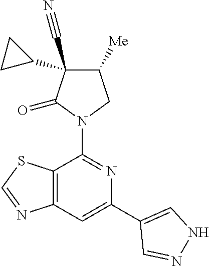 [SMILES: C[C@@H]1CN(c2nc(-c3cn[nH]c3)cc3ncsc23)C(=O)[C@]1(C#N)C1CC1]

To a solution of 4-chloro-6-(1H-pyrazol-4-yl)thiazolo[5,4-c]pyridine (Preparation 247, 1.35 g, 5.70 mmol) in dioxane (30 mL) was added (3R,4S)-3-cyclopropyl-4-methyl-2-oxopyrrolidine-3-carbonitrile (Preparation 21, 2.81 g, 17.11 mmol), K₂CO₃ (2.37 g, 17.11 mmol) and RuPhos Pd G3 (477 mg, 0.570 mmol) and the reaction was stirred at 100° C. for 2 h under N₂ atmosphere. The mixture was concentrated under reduced pressure to give the residue, which was purified by prep-HPLC-22 (gradient: 20 to 60%) to give (3R,4S)-1-(6-(1H-pyrazol-4-yl)thiazolo[5,4-c]pyridin-4-yl)-3-cyclopropyl-4-methyl-2-oxopyrrolidine-3-carbonitrile (385 mg, 16.9% yield) as yellow solid. LCMS m/z=365.2 [M+H]⁺

### Preparation 249

### 5-(1-methyl-1H-pyrazol-4-yl)benzo[d]thiazol-7-ol

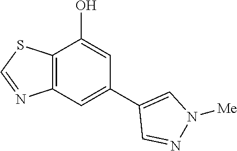 [SMILES: Cn1cc(-c2cc(O)c3scnc3c2)cn1]

To a mixture of 5-bromobenzo[d]thiazol-7-ol (Example I-119 &120, step 5, WO2020243423 140 mg, 0.608 mmol), 1-methyl-4-(4,4,5,5-tetramethyl-1,3,2-dioxaborolan-2-yl)-1H-pyrazole (189.9 mg, 0.913 mmol) and K₂CO₃ (252.3 mg, 1.83 mmol) in dioxane (2.5 mL) and water (0.5 mL) was added Pd(dtbpf)Cl₂ (39.66 mg, 0.061 mmol) at 20° C. The mixture was stirred at 90° C. for 2 h. The reaction mixture was concentrated to give a crude, which was purified by prep-HPLC-18 (gradient: 15 to 35%) to give 5-(1-methyl-1H-pyrazol-4-yl)benzo[d]thiazol-7-ol (20 mg, 14.2% yield) as a white solid. LCMS m/z=22.2 [M+H]⁺

### Preparation 250,

### 5-(1-(oxetan-3-yl)-1H-pyrazol-4-yl)benzo[d]thiazol-7-ol

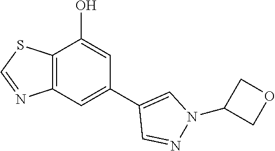 [SMILES: Oc1cc(-c2cnn(C3COC3)c2)cc2ncsc12]

To a mixture of 5-bromobenzo[d]thiazol-7-ol (90 mg, 0.391 mmol), 1-(oxetan-3-yl)-4-(4,4,5,5-tetramethyl-1,3,2-dioxaborolan-2-yl)-1H-pyrazole (146.8 mg, 0.587 mmol) and K₂CO₃ (162.2 mg, 1.17 mmol) in dioxane (2 mL) and water (0.4 mL) was added Pd(dtbpf)Cl₂ (50.99 mg, 0.0782 mmol) and the reaction was stirred at 90° C. for 2 h. The reaction mixture was concentrated to give a crude, which was purified by prep-HPLC-20 (Gradient: 20 to 40%) to give 5-(1-(oxetan-3-yl)-1H-pyrazol-4-yl)benzo[d]thiazol-7-ol (40 mg, 37.4% yield) as a brown solid. LCMS m/z=274.2 [M+H]⁺

### Preparation 251,

### 5-(1-(difluoromethyl)-1H-pyrazol-4-yl)benzo[d]thiazol-7-ol

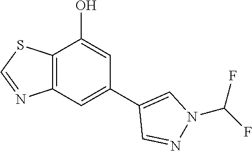 [SMILES: Oc1cc(-c2cnn(C(F)F)c2)cc2ncsc12]

5-(1-(Difluoromethyl)-1H-pyrazol-4-yl)benzo[d]thiazol-7-ol was obtained as a brown solid, 60 mg, 73.8% yield, from 5-bromobenzo[d]thiazol-7-ol and 1-(difluoromethyl)-4-(4,4,5,5-tetramethyl-1,3,2-dioxaborolan-2-yl)-1H-pyrazole, following a similar procedure to that described in Preparation 250. LCMS m/z=268.1 [M+H]⁺

### Preparation 252,

### 5-(1-methyl-1H-pyrazol-4-yl)benzo[d]thiazol-7-yl trifluoromethanesulfonate

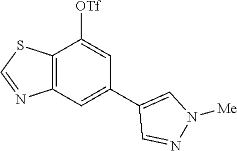 [SMILES: Cn1cc(-c2cc(OS(=O)(=O)C(F)(F)F)c3scnc3c2)cn1]

To a mixture of 5-(1-methyl-1H-pyrazol-4-yl)benzo[d]thiazol-7-ol (Preparation 249, 30 mg, 0.130 mmol) in DCM (3 mL) was added DIPEA (50.29 mg, 0.389 mmol) and Tf₂O (54.9 mg, 0.195 mmol) at 0° C. The mixture was stirred at 20° C. for 2 h. The reaction mixture was concentrated to give a crude, which was purified by TLC (PE/EtOAc=1/1) to give 5-(1-methyl-1H-pyrazol-4-yl)benzo[d]thiazol-7-yl trifluoromethanesulfonate (40 mg, 84.9% yield) as a light yellow solid. LCMS m/z=364.1 [M+H]⁺

### Preparation 253,

### 5-(1-(oxetan-3-yl)-1H-pyrazol-4-yl)benzo[d]thiazol-7-yl trifluoromethanesulfonate

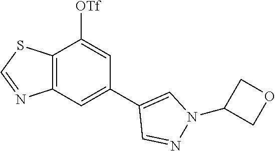 [SMILES: O=S(=O)(Oc1cc(-c2cnn(C3COC3)c2)cc2ncsc12)C(F)(F)F]

5-(1-(Oxetan-3-yl-H-pyrazol-4-yl)benzo[d]thiazol-7-yl trifluoromethanesulfonate was obtained as a white solid, 50 mg, 84.3% yield, from 5-(1-(oxetan-3-yl)-1H-pyrazol-4-yl)benzo[d]thiazol-7-ol (Preparation 250) following a similar procedure to that described in Preparation 252. LCMS m/z=406.0 [M+H]⁺

### Preparation 254,

### 5-(1-(difluoromethyl)-1H-pyrazol-4-yl)benzo[d]thiazol-7-yl trifluoromethanesulfonate

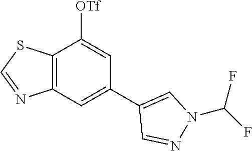 [SMILES: O=S(=O)(Oc1cc(-c2cnn(C(F)F)c2)cc2ncsc12)C(F)(F)F]

5-(1-(Difluoromethyl)-1H-pyrazol-4-yl)benzo[d]thiazol-7-yl trifluoromethanesulfonate was obtained as a white solid, 60 mg, 80.3% yield, from 5-(1-(difluoromethyl)-1H-pyrazol-4-yl)benzo[d]thiazol-7-ol (Preparation 251) following a similar procedure to that described in Preparation 252. LCMS m/z=400.0 [M+H]⁺

### Preparation 255,

### 2-(1-(bicyclo[1.1.1]pentan-1-yl)-1H-pyrazol-4-yl)-7-chlorofuro[3,2-b]pyridine

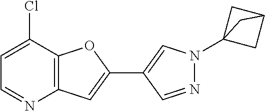 [SMILES: Clc1ccnc2cc(-c3cnn(C45CC(C4)C5)c3)oc12]

To a solution of 1-(bicyclo[1.1.1]pentan-1-yl)-4-(4,4,5,5-tetramethyl-1,3,2-dioxaborolan-2-yl)-1H-pyrazole (Preparation 236, 59 mg, 0.227 mmol) in dioxane (1 mL) and water (0.2 mL) was added K₂CO₃ (98.99 mg, 0.716 mmol) and 7-chloro-2-iodofuro[3,2-b]pyridine (66.72 mg, 239 mmol) at 20° C. Pd(dtbpf)Cl₂ (31.12 mg, 0.048 mmol) was added and the reaction was stirred at 90° C. under N₂ for 2 h. The mixture was concentrated under vacuum to give crude, which was purified by column chromatography on silica gel (PE/EtOAc=1/0 to 1/1) to give 2-(1-(bicyclo[1.1.1]pentan-1-yl)-1H-pyrazol-4-yl)-7-chlorofuro[3,2-b]pyridine (40 mg, 53.2% yield) as yellow oil. LCMS m/z=286.6 [M+H]⁺

### Preparation 256,

### 7-chloro-2-(1-isopropyl-1H-pyrazol-4-yl)furo[3,2-b]pyridine

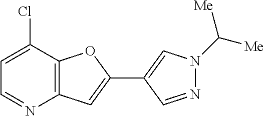 [SMILES: CC(C)n1cc(-c2cc3nccc(Cl)c3o2)cn1]

To a solution of 7-chloro-2-iodofuro[3,2-b]pyridine (140 mg, 0.501 mmol) in dioxane (2 mL) and water (0.4 mL) was added K₂CO₃ (207.71 mg, 1.50 mmol) and 1-isopropyl-4-(4,4,5,5-tetramethyl-1,3,2-dioxaborolan-2-yl)-1H-pyrazole (115.9 mg, 0.491 mmol) at 20° C. Pd(dtbpf)Cl₂ (32.65 mg, 0.050 mmol) was added and the reaction was stirred at 90° C. under N₂ for 2 h. The mixture was concentrated under vacuum to give crude, which was purified by chromatography column on silica gel (PE/EtOAc=1/0 to 3/1) to give 7-chloro-2-(1-isopropyl-1H-pyrazol-4-yl)furo[3,2-b]pyridine (120 mg, 81.5% yield) as a yellow solid. LCMS m/z=262.1 [M+H]⁺

### Preparation 257,

### 7-chloro-2-(1-cyclopropyl-1H-pyrazol-4-yl)furo[3,2-b]pyridine

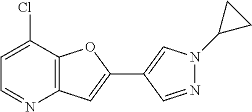 [SMILES: Clc1ccnc2cc(-c3cnn(C4CC4)c3)oc12]

7-Chloro-2-(1-cyclopropyl-1H-pyrazol-4-yl)furo[3,2-b]pyridine was obtained as a yellow solid, 120 mg, from 7-chloro-2-iodofuro[3,2-b]pyridine and 1-cyclopropyl-4-(4,4,5,5-tetramethyl-1,3,2-dioxaborolan-2-yl)-1H-pyrazole, following the method described in Preparation 256. LCMS m/z=260 [M+H]⁺

### Preparation 258,

### 7-chloro-2-(4-methyl-1H-pyrazol-1-yl)furo[3,2-b]pyridine

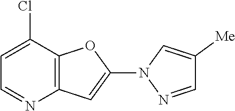 [SMILES: Cc1cnn(-c2cc3nccc(Cl)c3o2)c1]

To a mixture of 7-chloro-2-iodofuro[3,2-b]pyridine (200 mg, 0.716 mmol), 4-methyl-1H-pyrazole (117.5 mg, 1.43 mmol) and K₂CO₃ (296.7 mg, 2.15 mmol) in dioxane (5 mL) was added CuI (204.5 mg, 1.07 mmol) and N,N′-dimethylethane-1,2-diamine (94.63 mg, 1.07 mmol) and the reaction was stirred at 100° C. for 16 h. The reaction mixture was concentrated to give a crude, which was purified by prep-HPLC-21 (gradient: 40 to 68%) to give 7-chloro-2-(4-methyl-1H-pyrazol-1-yl)furo[3,2-b]pyridine (30 mg, 17.9% yield) as a white solid. LCMS m/z=234.0 [M+H]⁺

### Preparation 259,

### ((5-bromo-3-methoxy-2-methylphenyl)imino)-λ4-sulfanone

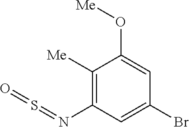 [SMILES: COc1cc(Br)cc(N=S=O)c1C]

To a solution of 5-bromo-3-methoxy-2-methylaniline (400 mg, 1.85 mmol) in toluene (20 mL) was added SOCl₂ (2.20 g, 18.51 mmol) at 20° C. and the mixture was stirred at 100° C. for 16 h. The mixture was concentrated under vacuum to give ((5-bromo-3-methoxy-2-methylphenyl)imino)-λ4-sulfanone (400 mg, crude) as yellow oil, which was used in the next step directly without further purification.

### Preparation 260,

### 6-bromo-4-methoxybenzo[c]isothiazole

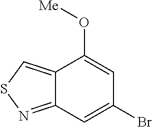 [SMILES: COc1cc(Br)cc2nscc12]

Part A: To a solution of methanesulfonamide (5.6 g, 58.87 mmol) in toluene (10 mL) was added SOCl₂ (10.51 g, 88.31 mmol) and the reaction mixture stirred at 90° C. for 12 h. The reaction mixture was concentrated in vacuo to give N-(oxo-λ4-sulfaneylidene)methanesulfonamide (6 g) as light brown oil, which was used for the next step without further purification.

Part B: To a solution of N-(oxo-λ4-sulfaneylidene)methanesulfonamide (215.4 mg, 1.53 mmol) and ((5-bromo-3-methoxy-2-methylphenyl)imino)-λ4-sulfanone (Preparation 259, 400 mg, 1.53 mmol) in toluene (30 mL) was added pyridine (603.5 mg, 7.63 mmol) at 15° C. and the reaction was stirred at 90° C. for 6 h. The reaction mixture was concentrated in vacuo to give crude product which was purified by silica gel chromatography (PE/EtOAc=25/1 to 6/1) to give 6-bromo-4-methoxybenzo[c]isothiazole (250 mg, 67.1% yield) as a white solid. LCMS m/z=244.1 [M+H]⁺

### Preparation 261,

### 6-bromobenzo[c]isothiazol-4-ol

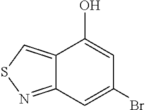 [SMILES: Oc1cc(Br)cc2nscc12]

6-Bromo-4-methoxybenzo[c]isothiazole (Preparation 260, 310 mg, 1.27 mmol) was added into HBr (15 mL) at 15° C. and the reaction mixture was stirred at 100° C. for 20 h. The reaction mixture was concentrated in vacuo to give 6-bromobenzo[c]isothiazol-4-ol (260 mg, crude) as a light gray solid, which was used for the next step without further purification. LCMS m/z=232.1 [M+H]⁺

### Preparation 262,

### 6-(1-methyl-1H-pyrazol-4-yl)benzo[c]isothiazol-4-ol

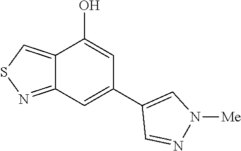 [SMILES: Cn1cc(-c2cc(O)c3csnc3c2)cn1]

6-(1-Methyl-1H-pyrazol-4-yl)benzo[c]isothiazol-4-ol was obtained, as a light gray solid, 120 mg, 47.8% yield, from 1-methyl-4-(4,4,5,5-tetramethyl-1,3,2-dioxaborolan-2-yl)-1H-pyrazole and 6-bromobenzo[c]isothiazol-4-ol (Preparation 261) following a similar method to that described in Preparation 249 except the crude product was purified by HPLC-16 (gradient: 17% to 37%). LCMS m/z=232.2 [M+H]⁺

### Preparation 263,

### 6-(1-methyl-1H-pyrazol-4-yl)benzo[c]isothiazol-4-yl trifluoromethanesulfonate

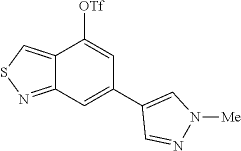 [SMILES: Cn1cc(-c2cc(OS(=O)(=O)C(F)(F)F)c3csnc3c2)cn1]

6-(1-Methyl-1H-pyrazol-4-yl)benzo[c]isothiazol-4-yl trifluoromethanesulfonate was obtained as a white solid, 100 mg, 63.7% yield, from 6-(1-methyl-1H-pyrazol-4-yl)benzo[c]isothiazol-4-ol and Tf₂O, following the method described in Preparation 249. LCMS m/z=364.1 [M+H]⁺

### Preparation 264,

### 6-bromopyrazolo[1,5-a]pyridin-4-yl trifluoromethanesulfonate

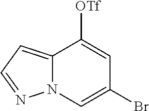 [SMILES: O=S(=O)(Oc1cc(Br)cn2nccc12)C(F)(F)F]

To a mixture of 6-bromopyrazolo[1,5-a]pyridin-4-ol (300 mg, 1.41 mmol) in DCM (15 mL) was added DIPEA (546.0 mg, 4.22 mmol) and Tf₂O (516.5 mg, 1.83 mmol) at 0° C. and the mixture was stirred at 25° C. for 2 h. The mixture was concentrated under vacuum to give the crude product, which was purified by silica gel column chromatography (PE/EtOAc=1/0 to 3/1) to give (6-bromopyrazolo[1,5-a]pyridin-4-yl) trifluoromethanesulfonate (390 mg, 80.3% yield) as colourless oil.

### Preparation 265

### (3R,4S)-1-(6-bromopyrazolo[1,5-a]pyridin-4-yl)-3-cyclopropyl-4-methyl-2-oxopyrrolidine-3-carbonitrile

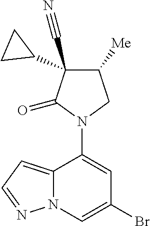 [SMILES: C[C@@H]1CN(c2cc(Br)cn3nccc23)C(=O)[C@]1(C#N)C1CC1]

To a solution of 6-bromopyrazolo[1,5-a]pyridin-4-yl trifluoromethanesulfonate (Preparation 264, 600 mg, 1.74 mmol) in toluene (10 mL) was added K₂CO₃ (720.9 mg, 5.22 mmol) and (3R,4S)-3-cyclopropyl-4-methyl-2-oxopyrrolidine-3-carbonitrile (Preparation 21, 279.8 mg, 1.70 mmol) at 20° C. Pd₂(dba)₃ (159.2 mg, 0.174 mmol) and Xantphos (100.6 mg, 0.174 mmol) was added and the reaction was stirred at 60° C. under N₂ for 4 h. The mixture was concentrated under vacuum, and the residue diluted with water (50 mL). The aqueous mixture was extracted with DCM (50 mL×3), the combined organic layers were washed with brine (100 mL), dried over Na₂SO₄, filtered and concentrated under vacuum. The crude product was purified by Prep-HPLC-18 (gradient: 46 to 66%)) to give (3R,4S)-1-(6-bromopyrazolo[1,5-a]pyridin-4-yl)-3-cyclopropyl-4-methyl-2-oxo-pyrrolidine-3-carbonitrile (110 mg, 16.7% yield) as a yellow solid. LCMS m/z=359.0 [M+H]⁺

### Preparation 266,

### (3R,4S)-1-(6-chloro-3-fluoropyrazolo[1,5-a]pyrazin-4-yl)-3-isopropyl-4-methyl-2-oxopyrrolidine-3-carbonitrile

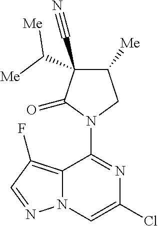 [SMILES: CC(C)[C@@]1(C#N)C(=O)N(c2nc(Cl)cn3ncc(F)c23)C[C@H]1C]

To a solution of (3R,4S)-3-isopropyl-4-methyl-2-oxopyrrolidine-3-carbonitrile (Preparation 209, 120 mg, 0.722 mmol) in THE (30 mL) was added NaH (40.42 mg, 1.01 mmol, 60% purity) and the reaction mixture was stirred at 20° C. for 20 min. 4,6-Dichloro-3-fluoropyrazolo[1,5-a]pyrazine (Preparation 124, 178.5 mg, 0.866 mmol) was added and the reaction mixture was stirred at 20° C. for 1 h. The reaction mixture was quenched with water (10 mL) and concentrated in vacuo. The crude product was purified by silica gel chromatography (PE/EtOAc=10/1 to 3/1) to give (3R,4S)-1-(6-chloro-3-fluoropyrazolo[1,5-a]pyrazin-4-yl)-3-isopropyl-4-methyl-2-oxopyrrolidine-3-carbonitrile (0.18 g, 66.8% yield) as a yellow solid. LCMS m/z=336.2 [M+H]⁺

### Preparation 267,

### (4-((3R,4S)-3-cyano-3-cyclopropyl-4-methyl-2-oxopyrrolidin-1-yl)pyrrolo[1,2-b]pyridazin-6-yl)boronic acid

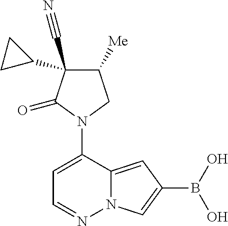 [SMILES: C[C@@H]1CN(c2ccnn3cc(B(O)O)cc23)C(=O)[C@]1(C#N)C1CC1]

A mixture of (3R,4S)-1-(6-bromopyrrolo[1,2-b]pyridazin-4-yl)-3-cyclopropyl-4-methyl-2-oxopyrrolidine-3-carbonitrile (Preparation 68, 10 g, 27.84 mmol), (BPin)₂ (14.14 g, 55.68 mmol), KOAc (8.20 g, 83.51 mmol) and Pd(dppf)Cl₂·DCM (2.27 g, 2.78 mmol) in dioxane (250 mL) was stirred at 90° C. under N₂ for 16 h. The reaction was filtered, the filtrate was concentrated and the residue was purified by column chromatography (PE/EtOAc=3/1) on silica gel. The product was further purified by prep-HPLC-22 (gradient: 29 to 69%) to give (4-((3R,4S)-3-cyano-3-cyclopropyl-4-methyl-2-oxopyrrolidin-1-yl)pyrrolo[1,2-b]pyridazin-6-yl)boronic acid (4.3 g, 47.7% yield) as a yellow solid. LCMS m/z=325.2 [M+H]⁺

### Preparation 268,

### (1-(4-bromo-1H-pyrazol-1-yl)cyclopropyl)methyl 4-methylbenzenesulfonate

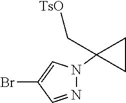 [SMILES: Cc1ccc(S(=O)(=O)OCC2(n3cc(Br)cn3)CC2)cc1]

To a solution of (1-(4-bromo-1H-pyrazol-1-yl)cyclopropyl)methanol (100 mg, 0.461 mmol) in DCM (6 mL) was added TEA (93.24 mg, 0.921 mmol) and DMAP (5.63 mg, 0.046 mmol) at 20° C. TsCl (105.4 mg, 0.553 mmol) was added and the reaction was stirred at 20° C. for 4 h. The mixture was concentrated in vacuo and the crude product was purified by chromatography column on silica gel (PE/EtOAc=1/0 to 1/1) to give (1-(4-bromo-1H-pyrazol-1-yl)cyclopropyl)methyl 4-methylbenzenesulfonate (160 mg, 92.6% yield) as a white solid. LCMS m/z=373.0 [M+H]⁺

### Preparation 269,

### 2-(1-(4-bromo-1H-pyrazol-1-yl)cyclopropyl)acetonitrile

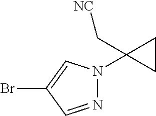 [SMILES: N#CCC1(n2cc(Br)cn2)CC1]

To a solution of (1-(4-bromo-1H-pyrazol-1-yl)cyclopropyl)methyl 4-methylbenzenesulfonate (Preparation 268, 100 mg, 0.269 mmol) in THF (10 mL) was added TMSCN (80.17 mg, 0.808 mmol) followed by TBAF (1 M in THF, 808.1 μL) dropwise and the reaction mixture stirred at 20° C. for 24 h. The mixture was poured into EtOAc (20 mL) and extracted with water (20 mL×5). The combined organic layers were washed with brine (20 mL), dried over Na₂SO₄, filtered and concentrated under vacuum to give 2-(1-(4-bromo-1H-pyrazol-1-yl)cyclopropyl)acetonitrile (70 mg, crude) as colorless oil. LCMS m/z=226.0 [M+H]⁺

### Preparation 270,

### 4-bromo-1-(1-methylpyrrolidin-3-yl)-1H-pyrazole

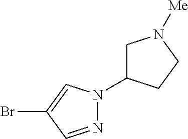 [SMILES: CN1CCC(n2cc(Br)cn2)C1]

To a solution of 4-bromo-1-(pyrrolidin-3-yl)-1H-pyrazole (170 mg, 0.787 mmol) in MeOH (5 mL) was added paraformaldehyde (118.0 mg, 3.93 mmol) and NaBH₃CN (500.2 mg, 2.36 mmol). The mixture was stirred under N₂ at 25° C. for 16 h. The mixture was concentrated and purified by prep-HPLC-17 (22% to 42% gradient) to give 4-bromo-1-(1-methylpyrrolidin-3-yl)-1H-pyrazole (150 mg, 82.9% yield) as a white solid. LCMS m/z=229.9 [M+H]⁺

### Preparation 271,

### tert-butyl 3-(4-(4-((3R,4S)-3-cyano-3-cyclopropyl-4-methyl-2-oxopyrrolidin-1-yl)thiazolo[5,4-c]pyridin-6-yl)-1H-pyrazol-1-yl)azetidine-1-carboxylate

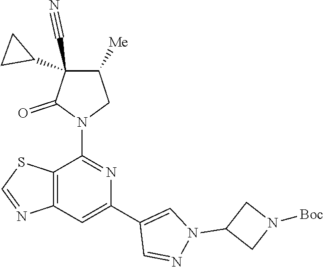 [SMILES: C[C@@H]1CN(c2nc(-c3cnn(C4CN(C(=O)OC(C)(C)C)C4)c3)cc3ncsc23)C(=O)[C@]1(C#N)C1CC1]

To a solution of (3R,4S)-1-(6-(1H-pyrazol-4-yl)thiazolo[5,4-c]pyridin-4-yl)-3-cyclopropyl-4-methyl-2-oxopyrrolidine-3-carbonitrile (Preparation 248, 20.0 mg, 0.055 mmol) in DMF (1 mL) was added Cs₂CO₃ (53.64 mg, 0.165 mmol) and tert-butyl 3-bromoazetidine-1-carboxylate (14.25 mg, 0.060 mmol) and the reaction was stirred at 90° C. for 1 h. The mixture was concentrated under vacuum to give the crude, which was purified by Prep-HPLC-20 (gradient: 43 to 93%) to give tert-butyl 3-(4-(4-((3R,4S)-3-cyano-3-cyclopropyl-4-methyl-2-oxopyrrolidin-1-yl)thiazolo[5,4-c]pyridin-6-yl)-1H-pyrazol-1-yl)azetidine-1-carboxylate (24.1 mg, crude) as a yellow solid. LCMS m/z=520.2 [M+H]⁺

### Preparation 272,

### tert-butyl 4-(4-(4-((3R,4S)-3-cyano-3-cyclopropyl-4-methyl-2-oxopyrrolidin-1-yl)pyrazolo[1,5-a]pyrazin-6-yl)-1H-pyrazol-1-yl)piperidine-1-carboxylate

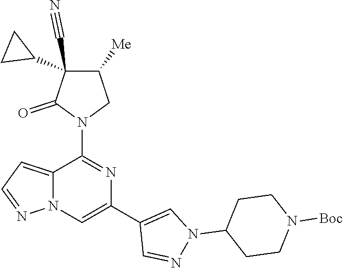 [SMILES: C[C@@H]1CN(c2nc(-c3cnn(C4CCN(C(=O)OC(C)(C)C)CC4)c3)cn3nccc23)C(=O)[C@]1(C#N)C1CC1]

N₂ was bubbled through a mixture of (3R,4S)-1-(6-chloropyrazolo[1,5-a]pyrazin-4-yl)-3-cyclopropyl-4-methyl-2-oxopyrrolidine-3-carbonitrile (Preparation 110, 200 mg, 0.633 mmol), tert-butyl 4-(4-(4,4,5,5-tetramethyl-1,3,2-dioxaborolan-2-yl)-1H-pyrazol-1-yl)piperidine-1-carboxylate (477.9 mg, 1.27 mmol), K₂CO₃ (262.6 mg, 1.90 mmol) and PEPPSI-IPr (43.16 mg, 0.063 mmol) in dioxane (10 mL) and water (2 mL) and the reaction was stirred at 90° C. for 2 h. The reaction mixture was concentrated in vacuo to give crude product, which was purified by silica gel (PE: EtOAc=1:1 to EtOAc) to give tert-butyl 4-(4-(4-((3R,4S)-3-cyano-3-cyclopropyl-4-methyl-2-oxopyrrolidin-1-yl)pyrazolo[1,5-a]pyrazin-6-yl)-1H-pyrazol-1-yl)piperidine-1-carboxylate (200 mg, 53.6% yield) as a pale yellow solid. LCMS m/z=475.2 [M+H]⁺

### Preparation 273,

### tert-butyl 3-(4-(4-((3R,4S)-3-cyano-3-cyclopropyl-4-methyl-2-oxopyrrolidin-1-yl)-3-fluoropyrazolo[1,5-a]pyrazin-6-yl)-1H-pyrazol-1-yl)azetidine-1-carboxylate

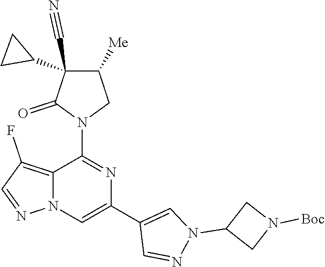 [SMILES: C[C@@H]1CN(c2nc(-c3cnn(C4CN(C(=O)OC(C)(C)C)C4)c3)cn3ncc(F)c23)C(=O)[C@]1(C#N)C1CC1]

tert-Butyl 3-(4-(4-((3R,4S)-3-cyano-3-cyclopropyl-4-methyl-2-oxopyrrolidin-1-yl)-3-fluoropyrazolo[1,5-a]pyrazin-6-yl)-1H-pyrazol-1-yl)azetidine-1-carboxylate was obtained as a pale yellow solid, 400 mg, 93% yield, from (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125) and tert-butyl 3-(4-(4,4,5,5-tetramethyl-1,3,2-dioxaborolan-2-yl)-1H-pyrazol-1-yl)azetidine-1-carboxylate, following the method described in Preparation 272. LCMS m/z=521.1 [M+H]⁺.

### Preparation 274

### tert-butyl 6-(4-(4-((3R,4S)-3-cyano-3-cyclopropyl-4-methyl-2-oxopyrrolidin-1-yl)pyrazolo[1,5-a]pyrazin-6-yl)-1H-pyrazol-1-yl)-2-azaspiro[3.3]heptane-2-carboxylate

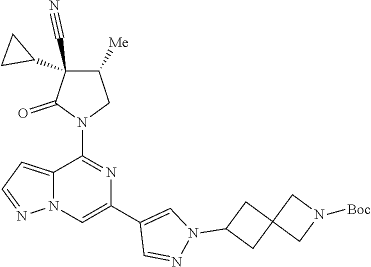 [SMILES: C[C@@H]1CN(c2nc(-c3cnn(C4CC5(C4)CN(C(=O)OC(C)(C)C)C5)c3)cn3nccc23)C(=O)[C@]1(C#N)C1CC1]

tert-Butyl 6-(4-(4-((3R,4S)-3-cyano-3-cyclopropyl-4-methyl-2-oxopyrrolidin-1-yl)pyrazolo[1,5-a]pyrazin-6-yl)-1H-pyrazol-1-yl)-2-azaspiro[3.3]heptane-2-carboxylate was obtained as a yellow oil, 110 mg, 78.9% yield, from (3R,4S)-1-(6-chloropyrazolo[1,5-a]pyrazin-4-yl)-3-cyclopropyl-4-methyl-2-oxopyrrolidine-3-carbonitrile (Preparation 110) and tert-butyl 6-(4-(4,4,5,5-tetramethyl-1,3,2-dioxaborolan-2-yl)-1H-pyrazol-1-yl)-2-azaspiro[3.3]heptane-2-carboxylate (Preparation 230), following the procedure described in Preparation 272. LCMS m/z=543.3 [M+H]⁺

### Preparation 275,

### tert-butyl 3-(4-(4-((3R,4S)-3-cyano-3-cyclopropyl-4-methyl-2-oxopyrrolidin-1-yl)pyrazolo[1,5-a]pyrazin-6-yl)-1H-pyrazol-1-yl)pyrrolidine-1-carboxylate

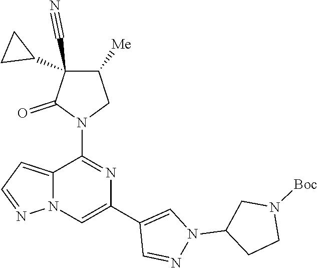 [SMILES: C[C@@H]1CN(c2nc(-c3cnn(C4CCN(C(=O)OC(C)(C)C)C4)c3)cn3nccc23)C(=O)[C@]1(C#N)C1CC1]

To a mixture of (3R,4S)-1-(6-chloropyrazolo[1,5-a]pyrazin-4-yl)-3-cyclopropyl-4-methyl-2-oxopyrrolidine-3-carbonitrile (Preparation 110, 150 mg, 0.475 mmol), tert-butyl 3-(4-(4,4,5,5-tetramethyl-1,3,2-dioxaborolan-2-yl)-1H-pyrazol-1-yl)pyrrolidine-1-carboxylate (258.9 mg, 0.713 mmol) and K₂CO₃ (197 mg, 1.43 mmol) in dioxane (8 mL) and water (1.6 mL) was added Pd(dtbpf)Cl₂ (32.37 mg, 0.0475 mmol) at 20° C. and the reaction mixture was stirred at 90° C. for 2 h. The reaction mixture was concentrated to give a crude, which was purified by column chromatography on silica gel eluted with PE: EtOAc (1:0 to 1:1) to give tert-butyl 3-(4-(4-((3R,4S)-3-cyano-3-cyclopropyl-4-methyl-2-oxopyrrolidin-1-yl)pyrazolo[1,5-a]pyrazin-6-yl)-1H-pyrazol-1-yl)pyrrolidine-1-carboxylate (200 mg, 81.5% yield) as yellow oil. LCMS m/z=517.2 [M+H]⁺

### Preparation 276,

### tert-butyl 6-(4-(4-((3R,4S)-3-cyano-3-cyclopropyl-4-methyl-2-oxopyrrolidin-1-yl)pyrrolo[1,2-b]pyridazin-6-yl)-1H-pyrazol-1-yl)-2-azaspiro[3.3]heptane-2-carboxylate

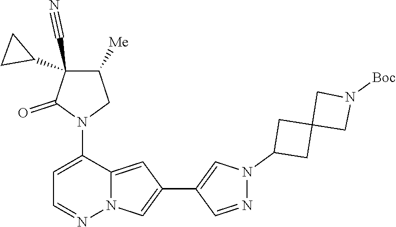 [SMILES: C[C@@H]1CN(c2ccnn3cc(-c4cnn(C5CC6(C5)CN(C(=O)OC(C)(C)C)C6)c4)cc23)C(=O)[C@]1(C#N)C1CC1]

tert-Butyl 6-(4-(4-((3R,4S)-3-cyano-3-cyclopropyl-4-methyl-2-oxopyrrolidin-1-yl)pyrrolo[1,2-b]pyridazin-6-yl)-1H-pyrazol-1-yl)-2-azaspiro[3.3]heptane-2-carboxylate was obtained as a yellow solid, 80 mg, 90% yield from tert-butyl 6-(4-bromo-1H-pyrazol-1-yl)-2-azaspiro[3.3]heptane-2-carboxylate and (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69), following the procedure described in Preparation 275. LCMS m/z=542.3 [M+H]⁺

### Preparation 277,

### tert-butyl 2-(1-(4-((3R,4S)-3-cyano-3-cyclopropyl-4-methyl-2-oxopyrrolidin-1-yl)pyrrolo[1,2-b]pyridazin-6-yl)-1H-pyrazol-4-yl)morpholine-4-carboxylate

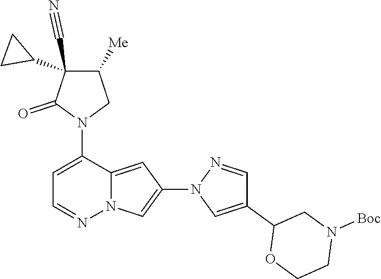 [SMILES: C[C@@H]1CN(c2ccnn3cc(-n4cc(C5CN(C(=O)OC(C)(C)C)CCO5)cn4)cc23)C(=O)[C@]1(C#N)C1CC1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 50 mg, 0.139 mmol) and tert-butyl 2-(1H-pyrazol-4-yl)morpholine-4-carboxylate (35.26 mg, 0.139 mmol) in dioxane (3 mL) was added K₂CO₃ (38.48 mg, 0.278 mmol), CuI (13.25 mg, 0.070 mmol) and N,N′-dimethylethane-1,2-diamine (6.13 mg, 0.067 mmol) at 20° C. The reaction mixture was stirred at 90° C. under N₂ for 12 h. The reaction mixture was concentrated in vacuum and the crude product was purified by prep-TLC (PE/EtOAc=1/1) to give tert-butyl 2-(1-(4-((3R,4S)-3-cyano-3-cyclopropyl-4-methyl-2-oxopyrrolidin-1-yl)pyrrolo[1,2-b]pyridazin-6-yl)-1H-pyrazol-4-yl)morpholine-4-carboxylate (32 mg, 43.3% yield) as a light yellow solid. LCMS m/z=532.3 [M+H]⁺

### Preparation 278

### tert-butyl 3-(4-(4-((3R,4S)-3-cyano-3-cyclopropyl-4-methyl-2-oxopyrrolidin-1-yl)pyrrolo[1,2-b]pyridazin-6-yl)-1H-pyrazol-1-yl)azetidine-1-carboxylate

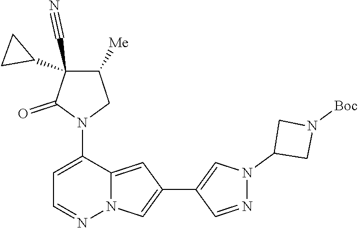 [SMILES: C[C@@H]1CN(c2ccnn3cc(-c4cnn(C5CN(C(=O)OC(C)(C)C)C5)c4)cc23)C(=O)[C@]1(C#N)C1CC1]

N₂ was bubbled through a mixture of (3R,4S)-1-(6-bromopyrrolo[1,2-b]pyridazin-4-yl)-3-cyclopropyl-4-methyl-2-oxopyrrolidine-3-carbonitrile (Preparation 68, 100 mg, 0.278 mmol), tert-butyl 3-(4-(4,4,5,5-tetramethyl-1,3,2-dioxaborolan-2-yl)-1H-pyrazol-1-yl)azetidine-1-carboxylate (194.4 mg, 0.557 mmol), K₂CO₃ (115.4 mg, 0.835 mmol) and Pd(dtbpf)Cl₂ (18.14 mg, 0.028 mmol) in dioxane (2 mL) and water (0.4 mL) and the reaction was stirred at 90° C. for 2 h. The reaction mixture was concentrated in vacuum and the crude product was purified by silica gel (PE/EtOAc=3:1 to EtOAc) to give tert-butyl 3-(4-(4-((3R,4S)-3-cyano-3-cyclopropyl-4-methyl-2-oxopyrrolidin-1-yl)pyrrolo[1,2-b]pyridazin-6-yl)-1H-pyrazol-1-yl)azetidine-1-carboxylate (120 mg, 77.4% yield) as a brown solid. LCMS m/z=502.2 [M+H]⁺

### Example 1

### (3R,4S)-3-cyclopropyl-4-methyl-1-(2-(1-methyl-1H-pyrazol-4-yl)-1H-pyrrolo[2,3-b]pyridin-4-yl)-2-oxopyrrolidine-3-carbonitrile

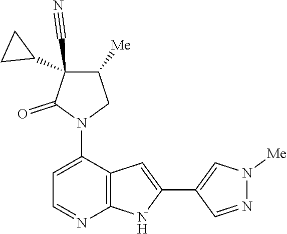 [SMILES: C[C@@H]1CN(c2ccnc3[nH]c(-c4cnn(C)c4)cc23)C(=O)[C@]1(C#N)C1CC1]

A solution of (3R,4S)-3-cyclopropyl-4-methyl-1-(2-(1-methyl-1H-pyrazol-4-yl)-1-tosyl-1H-pyrrolo[2,3-b]pyridin-4-yl)-2-oxopyrrolidine-3-carbonitrile (Preparation 53, 300 mg, 0.583 mmol) in TBAF (1 M, 5 mL) was stirred at 50° C. for 10 h. The reaction was concentrated under reduced pressure and the residue purified by column chromatography (6-100% EtOAc/PE) to give the crude product (140 mg) as a yellow solid. This was further purified by prep-HPLC-3 (gradient 26-56%) to give (3R,4S)-3-cyclopropyl-4-methyl-1-(2-(1-methyl-1H-pyrazol-4-yl)-1H-pyrrolo[2,3-b]pyridin-4-yl)-2-oxopyrrolidine-3-carbonitrile as a white solid (50 mg, 24%). LCMS m/z=361.2 [M+H]⁺; ¹H NMR (400 MHz, CDCl₃) δ:11.39 (br s, 1H), 8.20 (d, 1H), 7.88 (s, 1H), 7.77 (s, 1H), 7.21 (d, 1H), 6.52 (s, 1H), 4.12-4.07 (m, 1H), 4.00 (s, 3H), 3.90-3.85 (m, 1H), 3.17-3.07 (m, 1H), 1.45 (d, 3H), 1.22-1.20 (m, 1H), 0.84-0.75 (m, 4H).

### Example 2

### (R)-3-cyclopropyl-1-(2-(1-methyl-1H-pyrazol-4-yl)-1H-pyrrolo[2,3-b]pyridin-4-yl)-2-oxopyrrolidine-3-carbonitrile

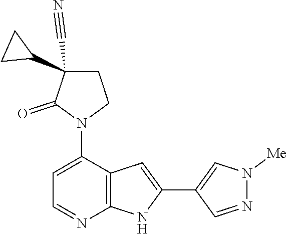 [SMILES: Cn1cc(-c2cc3c(N4CC[C@](C#N)(C5CC5)C4=O)ccnc3[nH]2)cn1]

To a solution of (R)-3-cyclopropyl-1-(2-(1-methyl-1H-pyrazol-4-yl)-1-tosyl-1H-pyrrolo[2,3-b]pyridin-4-yl)-2-oxopyrrolidine-3-carbonitrile (Preparation 55, 30 mg, 0.060 mmol) in THF (2 mL) was added TBAF (1 M, 60 μL) and the reaction stirred at 25° C. for 3 h. The crude was purified by prep-HPLC-3 (gradient 23-53%) to afford (R)-3-cyclopropyl-1-(2-(1-methyl-1H-pyrazol-4-yl)-1H-pyrrolo[2,3-b]pyridin-4-yl)-2-oxopyrrolidine-3-carbonitrile as a white solid (4.3 mg, 21%). LCMS m/z=347.2 [M+H]⁺; ¹H NMR (500 MHz, CDCl₃) δ: 11.47 (br s, 1H), 8.22 (d, 1H), 7.88 (s, 1H), 7.77 (s, 1H), 7.21 (d, 1H), 6.51 (s, 1H), 4.21-4.14 (m, 2H), 4.00 (s, 3H), 2.82-2.78 (m, 1H), 2.51-2.48 (m, 1H), 1.43-1.41 (m, 1H), 0.86-0.75 (m, 4H).

### Example 3

### (S)-3-cyclopropyl-1-(2-(2-methoxypyridin-4-yl)-1H-pyrrolo[2,3-b]pyridin-4-yl)-2-oxopyrrolidine-3-carbonitrile

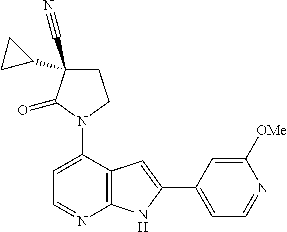 [SMILES: COc1cc(-c2cc3c(N4CC[C@@](C#N)(C5CC5)C4=O)ccnc3[nH]2)ccn1]

(S)-3-Cyclopropyl-1-(2-(2-methoxypyridin-4-yl)-1H-pyrrolo[2,3-b]pyridin-4-yl)-2-oxopyrrolidine-3-carbonitrile was prepared as a rose pink solid (33.4 mg, 36%), from (S)-3-cyclopropyl-1-(2-(2-methoxypyridin-4-yl)-1-tosyl-1H-pyrrolo[2,3-b]pyridin-4-yl)-2-oxopyrrolidine-3-carbonitrile (Preparation 56) using an analogous method to that described for Example 2, except HLPC-1 (gradient 24-54%) was used. LCMS/z=374.2 [M+H]⁺; ¹H NMR (400 MHz, CDCl₃) δ:12.59 (br s, 1H), 8.32 (d, 1H), 8.27 (d, 1H), 7.40 (d, 1H), 7.29 (d, 1H), 7.17 (s, 1H), 6.98 (s, 1H), 4.26-4.21 (m, 2H), 4.02 (s, 3H), 2.84-2.80 (m, 1H), 2.55-2.49 (m, 1H), 1.43-1.40 (m, 1H), 0.86-0.78 (m, 4H).

### Example 4

### (S)-3-cyclopropyl-1-(2-(1-methyl-1H-pyrazol-4-yl)-1H-pyrrolo[2,3-b]pyridin-4-yl)-2-oxopyrrolidine-3-carbonitrile

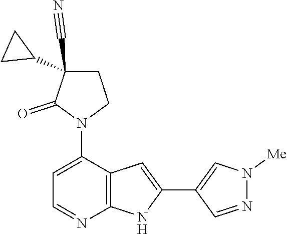 [SMILES: Cn1cc(-c2cc3c(N4CC[C@@](C#N)(C5CC5)C4=O)ccnc3[nH]2)cn1]

To a solution of (S)-3-cyclopropyl-1-(2-(1-methyl-1H-pyrazol-4-yl)-1-tosyl-1H-pyrrolo[2,3-b]pyridin-4-yl)-2-oxopyrrolidine-3-carbonitrile (Preparation 54, 860 mg, 1.72 mmol) in THF (3 mL) was added TBAF (1 M, 2.6 mL) and the reaction stirred at 25° C. for 16 h. The mixture was treated with H₂O (10 mL) and extracted with EtOAc (3×8 mL). The combined organics were washed with brine (10 mL), dried (Na₂SO₄) and evaporated to dryness in vacuo. The residue was purified by column chromatography (SiO₂, 50-100% EtOAc/PE) to give a white solid. The solid was slurried with MeOH (3 mL), filtered and the solid dried to give (S)-3-cyclopropyl-1-(2-(1-methyl-1H-pyrazol-4-yl)-1H-pyrrolo[2,3-b]pyridin-4-yl)-2-oxopyrrolidine-3-carbonitrile (210 mg). The filtrate was concentrated in vacuo and the residue purified by column chromatography (100% EtOAc) to give additional product (50 mg) as a light yellow solid. LCMS m/z=347.1 [M+H]⁺; ¹H NMR (500 MHz, MeOD-d₄) δ: 8.13 (d, 1H), 8.06 (s, 1H), 7.94 (s, 1H), 7.25 (d, 1H), 6.62 (s, 1H), 4.25-4.21 (m, 1H), 4.17-4.14 (m, 1H), 3.95 (s, 3H), 2.81-2.78 (m, 1H), 2.57-2.53 (m, 1H), 1.54-1.49 (m, 1H), 0.83-0.72 (m, 4H).

### Example 5

### (R)-3-cyclopropyl-1-(2-(2-methoxypyridin-4-yl)-1H-pyrrolo[2,3-b]pyridin-4-yl)-2-oxopyrrolidine-3-carbonitrile

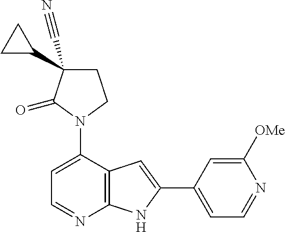 [SMILES: COc1cc(-c2cc3c(N4CC[C@](C#N)(C5CC5)C4=O)ccnc3[nH]2)ccn1]

To a solution of (R)-3-cyclopropyl-1-(2-(2-methoxypyridin-4-yl)-1-tosyl-1H-pyrrolo[2,3-b]pyridin-4-yl)-2-oxopyrrolidine-3-carbonitrile (Preparation 57, 100 mg, 0.190 mmol) in MeOH (30 mL) was added Mg (92.1 mg, 3.79 mmol) and the mixture stirred at 70° C. for 5 h. The mixture was filtered and the filtrate concentrated in vacuo. The residue was purified by prep-HPLC-3 (gradient 30-80%) to afford (R)-3-cyclopropyl-1-(2-(2-methoxypyridin-4-yl)-1H-pyrrolo[2,3-b]pyridin-4-yl)-2-oxopyrrolidine-3-carbonitrile as a white solid (6 mg, 9% yield). LCMS m/z=374.2 [M+H]⁺; ¹H NMR (500 MHz, CDCl₃) δ:11.99 (br s, 1H), 8.40 (d, 1H), 8.28 (d, 1H), 7.32 (d, 1H), 7.30-7.29 (m, 1H), 7.19 (s, 1H), 6.97 (s, 1H), 4.26-4.18 (m, 2H), 4.04 (s, 3H), 2.84-2.81 (m, 1H), 2.55-2.50 (m, 1H), 1.44-1.42 (m, 1H), 0.88-0.77 (m, 4H).

### Example 6

### (3R,4S)-3-cyclopropyl-4-methyl-1-(6-(1-methyl-1H-pyrazol-4-yl)-7H-pyrrolo[2,3-d]pyrimidin-4-yl)-2-oxopyrrolidine-3-carbonitrile

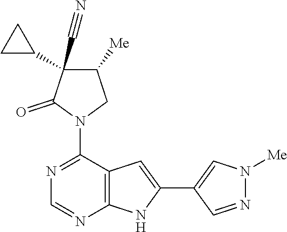 [SMILES: C[C@@H]1CN(c2ncnc3[nH]c(-c4cnn(C)c4)cc23)C(=O)[C@]1(C#N)C1CC1]

(3R,4S)-3-Cyclopropyl-4-methyl-1-(6-(1-methyl-1H-pyrazol-4-yl)-7H-pyrrolo[2,3-d]pyrimidin-4-yl)-2-oxopyrrolidine-3-carbonitrile was obtained as a white solid (22.6 mg, 22%), from (3R,4S)-3-cyclopropyl-4-methyl-1-(6-(1-methyl-1H-pyrazol-4-yl)-7-tosyl-7H-pyrrolo[2,3-d]pyrimidin-4-yl)-2-oxopyrrolidine-3-carbonitrile (Preparation 61), following a similar procedure to that described in Example 1. LCMS m/z=362.2 [M+H]⁺; ¹H NMR (500 MHz, CDCl₃) δ: 11.14 (br s, 1H), 8.58 (s, 1H), 7.92 (s, 1H), 7.89 (s, 1H), 7.05 (s, 1H), 4.34-4.30 (m, 1H), 3.99 (s, 3H), 3.96-3.91 (m, 1H), 3.12-3.07 (m, 1H), 1.44 (d, 3H), 1.20-1.18 (m, 1H), 0.83-0.73 (m, 4H).

### Example 7

### 1-(6-(1-methyl-1H-pyrazol-4-yl)pyrrolo[1,2-b]pyridazin-4-yl)-2-oxopyrrolidine-3-carbonitrile

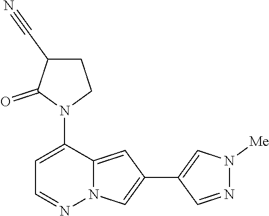 [SMILES: Cn1cc(-c2cc3c(N4CCC(C#N)C4=O)ccnn3c2)cn1]

To a solution of 1-(6-bromopyrrolo[1,2-b]pyridazin-4-yl)-2-oxopyrrolidine-3-carbonitrile (Preparation 66, 50 mg, 0.164 mmol) in dioxane (1 mL) and H₂O (0.1 mL) was added 1-methyl-4-(4,4,5,5-tetramethyl-1,3,2-dioxaborolan-2-yl)-1H-pyrazole (51.1 mg, 0.246 mmol), K₂CO₃ (67.9 mg, 0.492 mmol) and Pd(dppf)Cl₂ (12.0 mg, 0.0164 mmol) and the reaction stirred at 70° C. for 2 h under N₂. The cooled mixture was concentrated in vacuo and purified by prep-HPLC-3 (gradient 19-49%) to give 1-(6-(1-methyl-1H-pyrazol-4-yl)pyrrolo[1,2-b]pyridazin-4-yl)-2-oxopyrrolidine-3-carbonitrile as a grey solid (35 mg, 70%). LCMS m/z=307.1 [M+H]⁺; ¹H NMR (400 MHz, CDCl₃) δ: 8.00 (d, 1H), 7.90 (d, 1H), 7.75 (s, 1H), 7.62 (s, 1H), 6.75 (d, 1H), 6.59 (s, 1H), 4.17-4.13 (m, 2H), 3.98 (s, 3H), 3.81 (t, 1H), 2.80-2.73 (m, 1H), 2.66-2.60 (m, 1H).

### Example 8

### (S)-3-cyclopropyl-1-(6-(2-methoxypyridin-4-yl)pyrrolo[1,2-b]pyridazin-4-yl)-2-oxopyrrolidine-3-carbonitrile

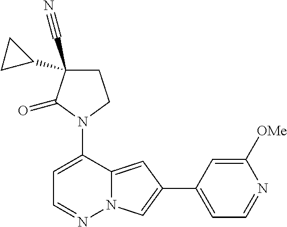 [SMILES: COc1cc(-c2cc3c(N4CC[C@@](C#N)(C5CC5)C4=O)ccnn3c2)ccn1]

To a solution of (S)-1-(6-bromopyrrolo[1,2-b]pyridazin-4-yl)-3-cyclopropyl-2-oxopyrrolidine-3-carbonitrile (Preparation 70, 100 mg, 0.290 mmol) and (2-methoxypyridin-4-yl)boronic acid (88.6 mg, 0.58 mmol) in dioxane (5 mL) and H₂O (0.5 mL) was added KF (50.5 mg, 0.87 mmol) and Pd(dppf)Cl₂ (21.2 mg, 0.029 mmol) and the reaction stirred at 70° C. for 4 h under N₂. The mixture was concentrated in vacuo and purified by prep-HPLC-3 (gradient 40-70%) to give (S)-3-cyclopropyl-1-(6-(2-methoxypyridin-4-yl)pyrrolo[1,2-b]pyridazin-4-yl)-2-oxopyrrolidine-3-carbonitrile as a yellow solid (55.4 mg, 51%). LCMS m/z=374.2 [M+H]⁺; ¹H NMR (400 MHz, CDCl₃) δ: 8.18 (d, 1H), 8.14 (s, 1H), 8.05 (d, 1H), 7.16-7.14 (m, 1H), 7.00 (s, 1H), 6.87-6.84 (m, 2H), 4.20-4.16 (m, 2H), 3.98 (s, 3H), 2.83-2.75 (m, 1H), 2.53-2.47 (m, 1H), 1.41-1.34 (m, 1H), 0.85-0.76 (m, 4H).

### Examples 9 to 12

The compounds in the following table were prepared from (S)-1-(6-bromopyrrolo[1,2-b]pyridazin-4-yl)-3-cyclopropyl-2-oxopyrrolidine-3-carbonitrile (Preparation 70) and the appropriate boronic acid or ester (RBY) following the procedure described in Example 8.

| Example | |
|---|---|
| No | Name/Structure/RBY/Data |
| 9 | (S)-3-cyclopropyl-1-(6-(1-methyl-1H-pyrazol-4-yl)pyrrolo[1,2-b]pyridazin- |
| | 4-yl)-2-oxopyrrolidine-3-carbonitrile |
| | 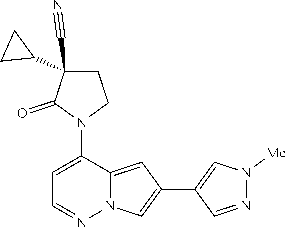 [SMILES: Cn1cc(-c2cc3c(N4CC[C@@](C#N)(C5CC5)C4=O)ccnn3c2)cn1] |
| | RBY: 1-methyl-4-(4,4,5,5-tetramethyl-1,3,2-dioxaborolan-2-yl)-1H- |
| | pyrazole |
| | 27.7 mg, 27.6 % yield as green solid. |
| | LCMS m/z = 347.2 [M + H]⁺; ¹H NMR (400 MHz, CDCl₃) δ: 7.98 (d, 1H), |
| | 7.89 (d, 1H), 7.72 (s, 1H), 7.60 (s, 1H), 6.78 (d, 1H), 6.59 (s, 1H), 4.16-4.12 |
| | (m, 2H), 3.94 (s, 3H), 2.79-2.74 (m, 1H), 2.50-2.45 (m, 1H), 1.39-1.35 (m, |
| | 1H), 0.84-0.74 (m, 4H). |
| 10^{A} | (S)-3-cyclopropyl-1-(6-(1-(difluoromethyl)-1H-pyrazol-4-yl)pyrrolo[1,2- |
| | b]pyridazin-4-yl)-2-oxopyrrolidine-3-carbonitrile |
| | 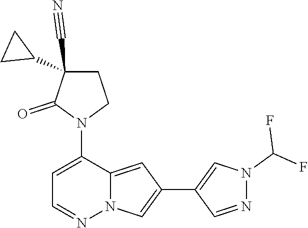 [SMILES: N#C[C@@]1(C2CC2)CCN(c2ccnn3cc(-c4cnn(C(F)F)c4)cc23)C1=O] |
| | RBY: 1-(difluoromethyl)-4-(4,4,5,5-tetramethyl-1,3,2-dioxaborolan-2-yl)- |
| | 1H-pyrazole |
| | 40.0 mg, 72.2 % yield as a green solid. |
| | LCMS m/z = 383.1 [M + H]⁺; ¹H NMR (500 MHz, CDCl₃) δ: 8.04-8.01 (m, |
| | 2H), 7.95 (s, 1H), 7.90 (s, 1H), 7.22 (t, 1H), 6.81 (d, 1H), 6.65 (s, 1H), 4.17- |
| | 4.12 (m, 2H), 2.81-2.75 (m, 1H), 2.51-2.46 (m, 1H), 1.40-1.35 (m, 1H), |
| | 0.85-0.74 (m, 4H). |
| 11^{A} | (S)-3-cyclopropyl-1-(6-(3-methylisoxazol-5-yl)pyrrolo[1,2-b]pyridazin-4- |
| | yl)-2-oxopyrrolidine-3-carbonitrile |
| | 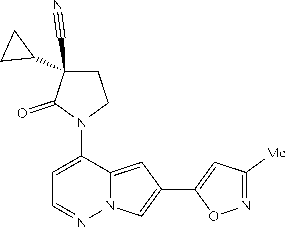 [SMILES: Cc1cc(-c2cc3c(N4CC[C@@](C#N)(C5CC5)C4=O)ccnn3c2)on1] |
| | RBY: 3-methyl-5-(4,4,5,5-tetramethyl-1,3,2-dioxaborolan-2-yl)isoxazole |
| | 18 mg, 35.8% yield as a green solid. |
| | LCMS m/z = 348.1 [M + H]⁺; ¹H NMR (500 MHz, CDCl₃) δ: 8.13 (s, 1H), |
| | 8.07 (d, 1H), 6.92 (d, 1H), 6.85 (s, 1H), 6.30 (s, 1H), 4.23-4.16 (m, 2H), |
| | 2.80-2.75 (m, 1H), 2.52-2.47 (m, 1H), 2.36 (s, 3H), 1.39-1.36 (m, 1H), |
| | 0.86-0.74 (m, 4H). |
| 12^{A} | (S)-3-cyclopropyl-1-(6-(3-fluoro-1-methyl-1H-pyrazol-4-yl)pyrrolo[1,2- |
| | b]pyridazin-4-yl)-2-oxopyrrolidine-3-carbonitrile |
| | 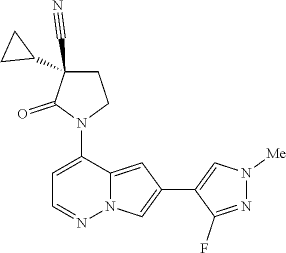 [SMILES: Cn1cc(-c2cc3c(N4CC[C@@](C#N)(C5CC5)C4=O)ccnn3c2)c(F)n1] |
| | RBY: 3-fluoro-1-methyl-4-(4,4,5,5-tetramethyl-1,3,2-dioxaborolan-2-yl)- |
| | 1H-pyrazole |
| | LCMS m/z = 365.1 [M + H]⁺; ¹H NMR (500 MHz, CDCl₃) δ: 8.00 (d, 1H), |
| | 7.95 (s, 1H), 7.47 (d, 1H), 6.81 (d, 1H), 6.63 (s, 1H), 4.19-4.12 (m, 2H), |
| | 3.82 (s, 3H), 2.80-2.74 (m, 1H), 2.51-2.46 (m, 1H), 1.39-1.36 (m, 1H), |
| | 0.84-0.74 (m, 4H). |

^{A}The reaction mixture was partitioned between water and EtOAc, the combined organic extracts washed with brine, dried over Na₂SO₄ and concentrated in vacuo

### Examples 13 to 33

The compounds in the following table were prepared from the appropriate bromide and Boronate following a similar procedure to that described in Example 8.

| Example | Name/Structure/Bromide/Data |
|---|---|
| 13 | (R)-3-cyclopropyl-1-(6-(2-methoxypyridin-4-yl)pyrrolo[1,2-b]pyridazin-4- |
| | yl)-2-oxopyrrolidine-3-carbonitrile |
| | 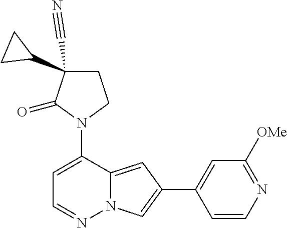 [SMILES: COc1cc(-c2cc3c(N4CC[C@](C#N)(C5CC5)C4=O)ccnn3c2)ccn1] |
| | Bromide: (R)-1-(6-bromopyrrolo[1,2-b]pyridazin-4-yl)-3-cyclopropyl-2- |
| | oxopyrrolidine-3-carbonitrile (Preparation 76); Boronate: (2- |
| | methoxypyridin-4-yl)boronic acid |
| | HPLC-3 (gradient 40-70%); Yellow solid (24 mg, 36%) |
| | LCMS m/z = 374.2 [M + H]⁺; ¹H NMR (500 MHz, CDCl₃) δ: 8.19 (d, 1H), |
| | 8.14 (s, 1H), 8.06 (d, 1H), 7.17-7.15 (m, 1H), 7.01 (s, 1H), 6.88-6.85 (m, |
| | 2H), 4.21-4.15 (m, 2H), 3.99 (s, 3H), 2.83-2.77 (m, 1H), 2.53-2.48 (m, 1H), |
| | 1.41-1.38 (m, 1H), 0.87-0.77 (m, 4H). |
| 14 | (R)-3-cyclopropyl-1-(6-(1-methyl-1H-pyrazol-4-yl)pyrrolo[1,2-b]pyridazin- |
| | 4-yl)-2-oxopyrrolidine-3-carbonitrile |
| | 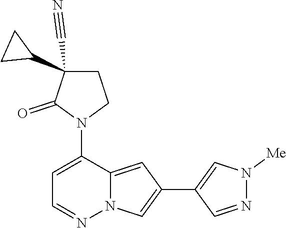 [SMILES: Cn1cc(-c2cc3c(N4CC[C@](C#N)(C5CC5)C4=O)ccnn3c2)cn1] |
| | Bromide: (R)-1-(6-bromopyrrolo[1,2-b]pyridazin-4-yl)-3-cyclopropyl-2- |
| | oxopyrrolidine-3-carbonitrile (Preparation 76) |
| | HPLC-3 (gradient 28-58%); Yellow solid (17 mg, 33%). |
| | LCMS m/z = 347.2 [M + H]⁺; ¹H NMR (500 MHz, CDCl₃) δ: 8.00 (d, 1H), |
| | 7.91 (d, 1H), 7.74 (s, 1H), 7.62 (s, 1H), 6.80 (d, 1H), 6.60 (d, 1H), 4.18- |
| | 4.13 (m, 2H), 3.96 (s, 3H), 2.81-2.75 (m, 1H), 2.51-2.47 (m, 1H), 1.40-1.37 |
| | (m, 1H), 0.85-0.76 (m, 4H). |
| 15 | (R)-3-cyclopropyl-3-(difluoromethyl)-1-(6-(1-methyl-1H-pyrazol-4- |
| | yl)pyrrolo[1,2-b]pyridazin-4-yl)pyrrolidin-2-one |
| | 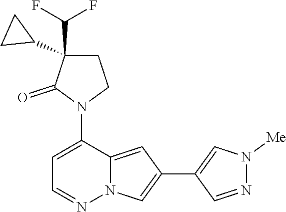 [SMILES: Cn1cc(-c2cc3c(N4CC[C@@](C(F)F)(C5CC5)C4=O)ccnn3c2)cn1] |
| | Bromide: (R)-1-(6-bromopyrrolo[1,2-b]pyridazin-4-yl)-3-cyclopropyl-3- |
| | (difluoromethyl)pyrrolidin-2-one (Preparation 77) |
| | HPLC-3 (gradient 40-70%); Yellow solid (50 mg, 54%). |
| | LCMS m/z = 372.1 [M + H]⁺; ¹H NMR (400 MHz, CDCl₃) δ: 7.96 (d, 1H), |
| | 7.88 (s, 1H), 7.74 (s, 1H), 7.61 (s, 1H), 6.68 (d, 1H), 6.56 (s, 1H), 6.12 (d, |
| | 1H), 4.12-4.06 (m, 1H), 4.00-3.94 (m, 4H), 2.69-2.61 (m, 1H), 2.06-2.02 |
| | (m, 1H), 1.18-1.15 (m, 1H), 0.77-0.72 (m, 2H), 0.59-0.58 (m, 1H), 0.36- |
| | 0.34 (m, 1H). |
| 16 | (3R,5R)-3-cyclopropyl-5-methyl-1-(6-(1-methyl-1H-pyrazol-4- |
| | yl)pyrrolo[1,2-b]pyridazin-4-yl)-2-oxopyrrolidine-3-carbonitrile |
| | 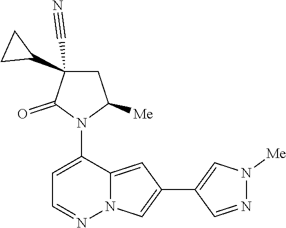 [SMILES: C[C@@H]1C[C@](C#N)(C2CC2)C(=O)N1c1ccnn2cc(-c3cnn(C)c3)cc12] |
| | Bromide: (3R,5R)-1-(6-bromopyrrolo[1,2-b]pyridazin-4-yl)-3-cyclopropyl- |
| | 5-methyl-2-oxopyrrolidine-3-carbonitrile (Preparation 78) |
| | HPLC-3 (gradient 30-50%); Green solid (30 mg, 37%). |
| | LCMS m/z = 361.1 [M + H]⁺; ¹H NMR (400 MHz, CDCl₃) δ: 8.02 (d, 1H), |
| | 7.91 (d, 1H), 7.73 (s, 1H), 7.61 (s, 1H), 6.51 (d, 1H), 6.50 (d, 1H), 4.57- |
| | 4.51 (m, 1H), 3.95 (s, 3H), 2.92-2.86 (m, 1H), 1.98-1.91 (m, 1H), 1.37-1.34 |
| | (m, 1H), 1.25 (d, 3H), 0.87-0.72 (m, 4H). |
| 17 | (3S,5R)-3-cyclopropyl-5-methyl-1-(6-(1-methyl-1H-pyrazol-4- |
| | yl)pyrrolo[1,2-b]pyridazin-4-yl)-2-oxopyrrolidine-3-carbonitrile |
| | 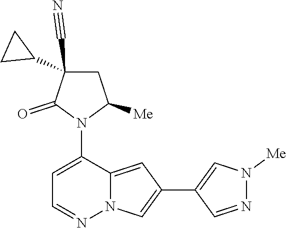 [SMILES: C[C@@H]1C[C@@](C#N)(C2CC2)C(=O)N1c1ccnn2cc(-c3cnn(C)c3)cc12] |
| | Bromide: (3S,5R)-1-(6-bromopyrrolo[1,2-b]pyridazin-4-yl)-3-cyclopropyl- |
| | 5-methyl-2-oxopyrrolidine-3-carbonitrile (Preparation 80) |
| | HPLC-3 (gradient 28-58%); Pale-green solid (38 mg, 40%). |
| | LCMS m/z = 361.1 [M + H]⁺; ¹H NMR (500 MHz, CDCl₃) δ: 8.04 (d, 1H), |
| | 7.92 (s, 1H), 7.73 (s, 1H), 7.62 (s, 1H), 6.57 (d, 1H), 6.50 (s, 1H), 4.58-4.50 |
| | (m, 1H), 3.96 (s, 3H), 2.75-2.70 (m, 1H), 2.44-2.40 (m, 1H), 1.39-1.37 (m, |
| | 1H), 1.29 (d, 3H), 0.82-0.73 (m, 4H). |
| 18 | (3S,5R)-3-cyclopropyl-5-ethyl-1-(6-(1-methyl-1H-pyrazol-4- |
| | yl)pyrrolo[1,2-b]pyridazin-4-yl)-2-oxopyrrolidine-3-carbonitrile |
| | 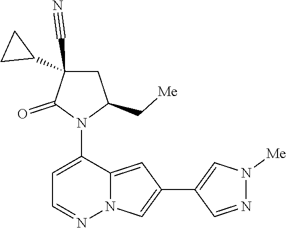 [SMILES: CC[C@@H]1C[C@@](C#N)(C2CC2)C(=O)N1c1ccnn2cc(-c3cnn(C)c3)cc12] |
| | Bromide: (3S,5R)-1-(6-bromopyrrolo[1,2-b]pyridazin-4-yl)-3-cyclopropyl- |
| | 5-ethyl-2-oxopyrrolidine-3-carbonitrile (Preparation 81) |
| | Green solid (10.7 mg, 13%). |
| | LCMS m/z = 375.1 [M + H]⁺; ¹H NMR (500 MHz, CDCl₃) δ: 8.02 (d, 1H), |
| | 7.91 (s, 1H), 7.73 (s, 1H), 7.61 (s, 1H), 6.56 (d, 1H), 6.49 (s, 1H), 4.38-4.32 |
| | (m, 1H), 3.95 (s, 3H), 2.67-2.63 (m, 1H), 2.48-2.43 (m, 1H), 1.65-1.61 (m, |
| | 1H), 1.52-1.49 (m, 1H), 1.37-1.35 (m, 1H), 0.88 (t, 3H), 0.80-0.74 (m, 4H). |
| 19 | (3S,5S)-3-cyclopropyl-5-ethyl-1-(6-(2-methyl-2H-imidazol-4- |
| | yl)pyrrolo[1,2-b]pyridazin-4-yl)-2-oxopyrrolidine-3-carbonitrile |
| | 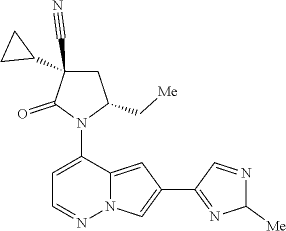 [SMILES: CC[C@H]1C[C@@](C#N)(C2CC2)C(=O)N1c1ccnn2cc(C3=NC(C)N=C3)cc12] |
| | Bromide: (3S,5S)-1-(6-bromopyrrolo[1,2-b]pyridazin-4-yl)-3-cyclopropyl- |
| | 5-ethyl-2-oxopyrrolidine-3-carbonitrile (Preparation 82) |
| | 14 mg, 35 % yield as a green solid. |
| | LCMS m/z = 375.1 [M + H]⁺ |
| | ¹H NMR (400 MHz, CDCl₃) δ: 8.02 (d, 1H), 7.91 (d, 1H), 7.73 (s, 1H), |
| | 7.61 (s, 1H), 6.48-6.52 (m, 2H), 4.37-4.42 (m, 1H), 3.95 (s, 3H), 2.82-2.88 |
| | (m, 1H), 1.95-2.01 (m, 1H), 1.77-1.78 (m, 1H), 1.36-1.42 (m, 2H), 0.72- |
| | 0.90 (m, 7H). |
| 20 | (3S,4S)-4-methyl-1-(6-(1-methyl-1H-pyrazol-4-yl)pyrrolo[1,2-b]pyridazin- |
| | 4-yl)-3-(oxetan-3-yl)-2-oxopyrrolidine-3-carbonitrile |
| | 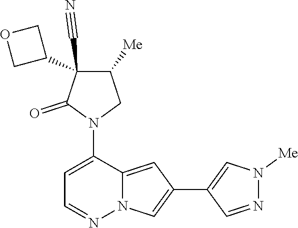 [SMILES: C[C@@H]1CN(c2ccnn3cc(-c4cnn(C)c4)cc23)C(=O)[C@]1(C#N)C1COC1] |
| | Bromide: (3S,4S)-1-(6-bromopyrrolo[1,2-b]pyridazin-4-yl)-4-methyl-3- |
| | (oxetan-3-y1)-2-oxopyrrolidine-3-carbonitrile (Preparation 83) |
| | HPLC-3 (gradient 24-54%) |
| | LCMS m/z = 377.1 [M + H]⁺; ¹H NMR (400 MHz, CDCl₃) δ: 7.97 (d, 1H), |
| | 7.90 (s, 1H), 7.73 (s, 1H), 7.62 (s, 1H), 6.74 (d, 1H), 6.55 (s, 1H), 5.14-5.12 |
| | (m, 1H), 4.94-4.92 (m, 1H), 4.88-4.79 (m, 2H), 4.19-4.17 (m, 1H), 3.95 (s, |
| | 3H), 3.73-3.71 (m, 1H), 3.62-3.61 (m, 1H), 3.12-3.07 (m, 1H), 1.15 (d, 3H). |
| 21 | (3S,4S)-3-cyclopropyl-4-methyl-1-(6-(1-methyl-1H-pyrazol-4- |
| | yl)pyrrolo[1,2-b]pyridazin-4-yl)-2-oxopyrrolidine-3-carbonitrile |
| | 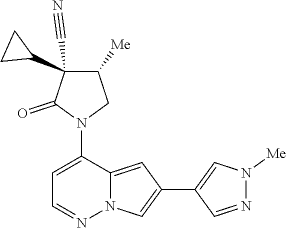 [SMILES: C[C@@H]1CN(c2ccnn3cc(-c4cnn(C)c4)cc23)C(=O)[C@@]1(C#N)C1CC1] |
| | Bromide: (3S,4S)-1-(6-bromopyrrolo[1,2-b]pyridazin-4-yl)-3-cyclopropyl- |
| | 4-methyl-2-oxopyrrolidine-3-carbonitrile (Preparation 84) |
| | HPLC-3 (gradient 31-61%: Green solid (55 mg, 78%). |
| | LCMS m/z = 361.1 [M + H]⁺; ¹H NMR (500 MHz, CDCl₃) δ: 7.97 (d, 1H), |
| | 7.89 (s, 1H), 7.73 (s, 1H), 7.61 (s, 1H), 6.79 (d, 1H), 6.58 (s, 1H), 4.17-4.12 |
| | (m, 1H), 3.95 (s, 3H), 3.81-3.76 (m, 1H), 2.73-2.65 (m, 1H), 1.45 (d, 3H), |
| | 1.25-1.21 (m, 1H), 0.85-0.72 (m, 4H). |
| 22 | (3R,4R)-3-cyclopropyl-4-methyl-1-(6-(1-methyl-1H-pyrazol-4- |
| | yl)pyrrolo[1,2-b]pyridazin-4-yl)-2-oxopyrrolidine-3-carbonitrile |
| | 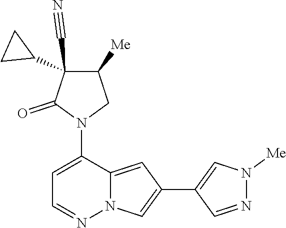 [SMILES: C[C@H]1CN(c2ccnn3cc(-c4cnn(C)c4)cc23)C(=O)[C@]1(C#N)C1CC1] |
| | Bromide: (3R,4R)-1-(6-bromopyrrolo[1,2-b]pyridazin-4-yl)-3-cyclopropyl- |
| | 4-methyl-2-oxopyrrolidine-3-carbonitrile (Preparation 85) |
| | HPLC-4 (gradient 30-60%); Yellow solid (40.3 mg, 57%). |
| | LCMS m/z = 361.0 [M + H]⁺; ¹H NMR (500 MHz, CDCl₃) δ: 7.97 (d, 1H), |
| | 7.89 (s, 1H), 7.73 (s, 1H), 7.61 (s, 1H), 6.79 (d, 1H), 6.58 (d, 1H), 4.17-4.12 |
| | (m, 1H), 3.95 (s, 3H), 3.80-3.76 (m, 1H), 2.73-2.67 (m, 1H), 1.45 (d, 3H), |
| | 1.24-1.21 (m, 1H), 0.85-0.73 (m, 4H). |
| 23 | (3R,4S)-3-cyclopropyl-4-ethyl-1-(6-(1-methyl-1H-pyrazol-4- |
| | yl)pyrrolo[1,2-b]pyridazin-4-yl)-2-oxopyrrolidine-3-carbonitrile |
| | 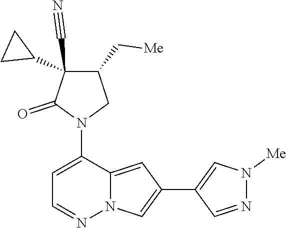 [SMILES: CC[C@@H]1CN(c2ccnn3cc(-c4cnn(C)c4)cc23)C(=O)[C@]1(C#N)C1CC1] |
| | Bromide: (3R,4S)-1-(6-bromopyrrolo[1,2-b]pyridazin-4-yl)-3-cyclopropyl- |
| | 4-ethyl-2-oxopyrrolidine-3-carbonitrile (Preparation 86) |
| | HPLC-3 (gradient 34-64%); Green solid (51.1 mg, 54%). |
| | LCMS m/z = 375.2 [M + H]⁺; ¹H NMR (500 MHz, CDCl₃) δ: 7.98 (d, 1H), |
| | 7.89 (d, 1H), 7.74 (s, 1H), 7.62 (s, 1H), 6.82 (d, 1H), 6.58 (d, 1H), 4.11- |
| | 4.08 (m, 1H), 3.96 (s, 3H), 3.88-3.83 (m, 1H), 2.94-2.89 (m, 1H), 2.05-2.00 |
| | (m, 1H), 1.81-1.78 (m, 1H), 1.20-1.16 (m, 1H), 1.15-1.13 (m, 3H), 0.81- |
| | 0.77 (m, 4H). |
| 24 | (3R,4R)-3-cyclopropyl-4-ethyl-1-(6-(1-methyl-1H-pyrazol-4- |
| | yl)pyrrolo[1,2-b]pyridazin-4-yl)-2-oxopyrrolidine-3-carbonitrile |
| | 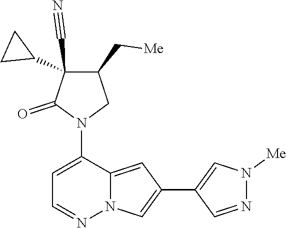 [SMILES: CC[C@H]1CN(c2ccnn3cc(-c4cnn(C)c4)cc23)C(=O)[C@]1(C#N)C1CC1] |
| | Bromide: (3R,4R)-1-(6-bromopyrrolo[1,2-b]pyridazin-4-yl)-3-cyclopropyl- |
| | 4-ethyl-2-oxopyrrolidine-3-carbonitrile (Preparation 87) |
| | HPLC-3 (gradient 35-65%); Light-green solid (33 mg, 31%). |
| | LCMS m/z = 375.1 [M + H]⁺; ¹H NMR (400 MHz, CDCl₃) δ: 7.99 (d, 1H), |
| | 7.91 (d, 1H), 7.75 (s, 1H), 7.63 (s, 1H), 6.78 (d, 1H), 6.59 (s, 1H), 4.19-4.14 |
| | (m, 1H), 3.96 (s, 3H), 3.85-3.80 (m, 1H), 2.52-2.49 (m, 1H), 2.03-2.00 (m, |
| | 1H), 1.82-1.73 (m, 1H), 1.26-1.23 (m, 1H), 1.12-1.09 (m, 3H), 0.87-0.77 |
| | (m, 4H). |
| 25 | (3S,4R)-3-cyclopropyl-4-ethyl-1-(6-(1-methyl-1H-pyrazol-4- |
| | yl)pyrrolo[1,2-b]pyridazin-4-yl)-2-oxopyrrolidine-3-carbonitrile |
| | 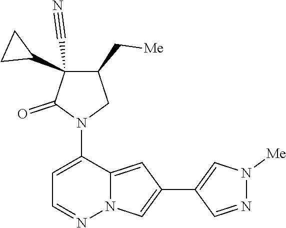 [SMILES: CC[C@H]1CN(c2ccnn3cc(-c4cnn(C)c4)cc23)C(=O)[C@@]1(C#N)C1CC1] |
| | Bromide: (3S,4R)-1-(6-bromopyrrolo[1,2-b]pyridazin-4-yl)-3-cyclopropyl- |
| | 4-ethyl-2-oxopyrrolidine-3-carbonitrile (Preparation 92) |
| | HPLC-3 (gradient 35-65%); Green solid (35 mg, 50%). |
| | LCMS m/z = 375.1 [M + H]⁺; ¹H NMR (400 MHz, CDCl₃) δ: 7.98 (d, 1H), |
| | 7.89 (d, 1H), 7.73 (s, 1H), 7.62 (s, 1H), 6.82 (d, 1H), 6.58 (s, 1H), 4.12-4.07 |
| | (m, 1H), 3.96 (s, 3H), 3.88-3.83 (m, 1H), 2.93-2.86 (m, 1H), 2.04-1.99 (m, |
| | 1H), 1.83-1.80 (m, 1H), 1.22-1.20 (m, 1H), 1.15 (t, 3H), 0.81-0.76 (m, 4H). |
| 26 | (3S,4S)-3-cyclopropyl-4-ethyl-1-(6-(1-methyl-1H-pyrazol-4-yl)pyrrolo[1,2- |
| | b]pyridazin-4-yl)-2-oxopyrrolidine-3-carbonitrile |
| | 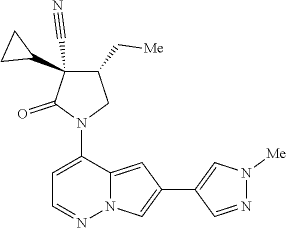 [SMILES: CC[C@@H]1CN(c2ccnn3cc(-c4cnn(C)c4)cc23)C(=O)[C@@]1(C#N)C1CC1] |
| | Bromide: (3S,4S)-1-(6-bromopyrrolo[1,2-b]pyridazin-4-yl)-3-cyclopropyl- |
| | 4-ethyl-2-oxopyrrolidine-3-carbonitrile (Preparation 93) |
| | HPLC-3 (gradient 35-65%); Yellow solid (30 mg, 31%). |
| | LCMS m/z = 375.2 [M + H]⁺; ¹H NMR (400 MHz, CDCl₃) δ: 7.98 (d, 1H), |
| | 7.90 (s, 1H), 7.74 (s, 1H), 7.62 (s, 1H), 6.77 (d, 1H), 6.58 (s, 1H), 4.18-4.14 |
| | (m, 1H), 3.96 (s, 3H), 3.85-3.79 (m, 1H), 2.51-2.49 (m, 1H), 2.03-2.00 (m, |
| | 1H), 1.79-1.76 (m, 1H), 1.34-1.24 (m, 1H), 1.11 (t, 3H), 0.86-0.74 (m, 4H). |
| 27 | (R)-2-(3-cyclopropyl-1-(6-(1-methyl-1H-pyrazol-4-yl)pyrrolo[1,2- |
| | b]pyridazin-4-yl)-2-oxopyrrolidin-3-yl)acetonitrile |
| | 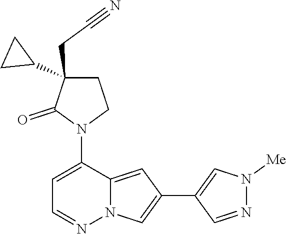 [SMILES: Cn1cc(-c2cc3c(N4CC[C@@](CC#N)(C5CC5)C4=O)ccnn3c2)cn1] |
| | Bromide: (R)-2-(1-(6-bromopyrrolo[1,2-b]pyridazin-4-yl)-3-cyclopropyl-2- |
| | oxopyrrolidin-3-yl)acetonitrile (Preparation 88) |
| | Yellow solid (17.1 mg, 34%). |
| | LCMS m/z = 361.2 [M + H]⁺; ¹H NMR (500 MHz, CDCl₃) δ: 7.96 (d, 1H), |
| | 7.88 (d, 1H), 7.73 (s, 1H), 7.60 (s, 1H), 6.75 (d, 1H), 6.60 (s, 1H), 4.18-4.14 |
| | (m, 1H), 4.00-3.97 (m, 1H), 3.95 (s, 3H), 2.82 (s, 2H), 2.46-2.41 (m, 1H), |
| | 2.32-2.30 (m, 1H), 1.20-1.18 (m, 1H), 0.74-0.72 (m, 1H), 0.61-0.57 (m, |
| | 2H), 0.42-0.40 (m, 1H). |
| 28 | (3R,4S)-3-cyclopropyl-1-(7-fluoro-6-(1-methyl-1H-pyrazol-4- |
| | yl)pyrrolo[1,2-b]pyridazin-4-yl)-4-methyl-2-oxopyrrolidine-3-carbonitrile |
| | 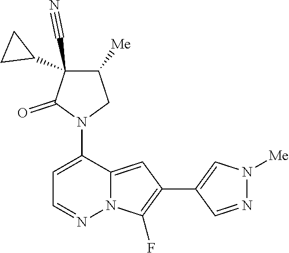 [SMILES: C[C@@H]1CN(c2ccnn3c(F)c(-c4cnn(C)c4)cc23)C(=O)[C@]1(C#N)C1CC1] |
| | Bromide: (3R,4S)-1-(6-bromo-7-fluoropyrrolo[1,2-b]pyridazin-4-yl)-3- |
| | cyclopropyl-4-methyl-2-oxopyrrolidine-3-carbonitrile (Preparation 94) |
| | Yellow solid (10 mg, 25%). |
| | LCMS m/z = 379.1 [M + H]⁺; ¹H NMR (500 MHz, CDCl₃) δ: 8.03 (d, 1H), |
| | 7.88-7.74 (m, 2H), 6.78-6.74 (m, 1H), 6.56 (d, 1H), 4.13-4.09 (m, 1H), 3.99 |
| | (s, 3H), 3.88-3.84 (m, 1H), 3.11-3.09 (m, 1H), 1.44 (d, 3H), 1.18-1.17 (m, |
| | 1H), 0.82-0.77 (m, 4H). |
| 29 | (3S,4R)-3-cyclopropyl-4-methyl-1-(6-(1-methyl-1H-pyrazol-4- |
| | yl)pyrrolo[1,2-b]pyridazin-4-yl)-2-oxopyrrolidine-3-carbonitrile |
| | 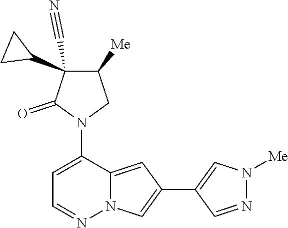 [SMILES: C[C@H]1CN(c2ccnn3cc(-c4cnn(C)c4)cc23)C(=O)[C@@]1(C#N)C1CC1] |
| | Bromide: (3S,4R)-1-(6-bromopyrrolo[1,2-b]pyridazin-4-yl)-3-cyclopropyl- |
| | 4-methyl-2-oxopyrrolidine-3-carbonitrile (Preparation 127) |
| | Boronate: 1-methyl-4-(4,4,5,5-tetramethyl-1,3,2-dioxaborolan-2-yl)-1H- |
| | pyrazole |
| | HPLC-3 (gradient 40-70%); Yellow solid (24 mg, 36%). |
| | LCMS m/z = 360.9 [M + H]⁺; ¹H NMR (500 MHz, CDCl₃) δ: 8.01 (d, 1H), |
| | 7.79 (d, 1H), 6.95 (d, 1H), 6.61 (d, 1H), 4.09-4.06 (m, 1H), 3.85-3.80 (m, |
| | 1H), 3.10-3.03 (m, 1H), 1.42 (d, 3H), 1.18-1.13 (m, 1H), 0.81-0.76 (m, 4H). |
| 30 | (3R,5S)-3-cyclopropyl-5-methyl-1-(6-(1-methyl-1H-pyrazol-4- |
| | yl)pyrrolo[1,2-b]pyridazin-4-yl)-2-oxopyrrolidine-3-carbonitrile |
| | 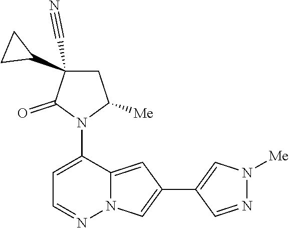 [SMILES: C[C@H]1C[C@](C#N)(C2CC2)C(=O)N1c1ccnn2cc(-c3cnn(C)c3)cc12] |
| | Bromide: (3R,5S)-1-(6-bromopyrrolo[1,2-b]pyridazin-4-yl)-3-cyclopropyl- |
| | 5-methyl-2-oxopyrrolidine-3-carbonitrile (Preparation 89) |
| | Boronate: 1-methyl-4-(4,4,5,5-tetramethyl-1,3,2-dioxaborolan-2-yl)-1H- |
| | pyrazole |
| | HPLC-3 (gradient 29-59%); Green solid (15 mg, 19%). |
| | LCMS m/z = 361.1 [M + H]⁺; ¹H NMR (400 MHz, CDCl₃) δ: 8.02 (d, 1H), |
| | 7.91 (s, 1H), 7.76 (s, 1H), 7.61 (s, 1H), 6.57-6.49 (m, 2H), 4.58-4.49 (m, |
| | 1H), 3.95 (s, 3H), 2.75-2.69 (m, 1H), 2.44-2.38 (m, 1H), 1.40-1.34 (m, 1H), |
| | 1.28 (d, 3H), 0.81-0.74 (m, 4H). |
| 31 | (3S,5S)-3-cyclopropyl-5-methyl-1-(6-(1-methyl-1H-pyrazol-4- |
| | yl)pyrrolo[1,2-b]pyridazin-4-yl)-2-oxopyrrolidine-3-carbonitrile |
| | 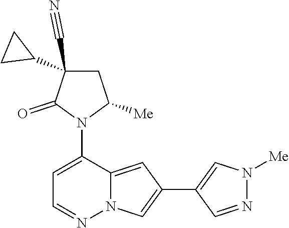 [SMILES: C[C@H]1C[C@@](C#N)(C2CC2)C(=O)N1c1ccnn2cc(-c3cnn(C)c3)cc12] |
| | Bromide: (3S,5S)-1-(6-bromopyrrolo[1,2-b]pyridazin-4-yl)-3-cyclopropyl- |
| | 5-methyl-2-oxopyrrolidine-3-carbonitrile (Preparation 79) |
| | Boronate: 1-methyl-4-(4,4,5,5-tetramethyl-1,3,2-dioxaborolan-2-y1)-1H- |
| | pyrazole |
| | HPLC-3 (gradient 29-59%); Green solid (13 mg, 32%). |
| | LCMS m/z = 361.1 [M + H]⁺; ¹H NMR (500 MHz, CDCl₃) δ: 8.02 (d, 1H), |
| | 7.92 (s, 1H), 7.73 (s, 1H), 7.61 (s, 1H), 6.53-6.48 (m, 2H), 4.58-4.50 (m, |
| | 1H), 3.95 (s, 3H), 2.91-2.87 (m, 1H), 1.97-1.92 (m, 1H), 1.40-1.35 (m, 1H), |
| | 1.26 (d, 3H), 0.89-0.73 (m, 4H). |
| 32 | (3R,5S)-3-cyclopropyl-5-ethyl-1-(6-(1-methyl-1H-pyrazol-4- |
| | yl)pyrrolo[1,2-b]pyridazin-4-yl)-2-oxopyrrolidine-3-carbonitrile |
| | 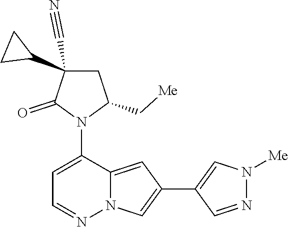 [SMILES: CC[C@H]1C[C@](C#N)(C2CC2)C(=O)N1c1ccnn2cc(-c3cnn(C)c3)cc12] |
| | Bromide: (3R,5R)-1-(6-bromopyrrolo[1,2-b]pyridazin-4-yl)-3-cyclopropyl- |
| | 5-ethyl-2-oxopyrrolidine-3-carbonitrile (Preparation 90) |
| | Boronate: 1-methyl-4-(4,4,5,5-tetramethyl-1,3,2-dioxaborolan-2-yl)-1H- |
| | pyrazole |
| | HPLC-3 (gradient 32-62%); Green solid (8.8 mg, 7%). |
| | LCMS m/z = 375.1 [M + H]⁺; ¹H NMR (500 MHz, CDCl₃) δ: 8.03 (d, 1H), |
| | 7.92 (s, 1H), 7.74 (s, 1H), 7.62 (s, 1H), 6.57 (d, 1H), 6.50 (s, 1H), 4.39-4.33 |
| | (m, 1H), 3.96 (s, 3H), 2.68-2.64 (m, 1H), 2.49-2.45 (m, 1H), 1.79-1.73 (m, |
| | 1H), 1.51-1.47 (m, 1H), 1.38-1.36 (m, 1H), 0.89 (t, 3H), 0.81-0.73 (m, 4H). |
| 33 | (3R,5R)-3-cyclopropyl-5-ethyl-1-(6-(1-methyl-1H-pyrazol-4- |
| | yl)pyrrolo[1,2-b]pyridazin-4-yl)-2-oxopyrrolidine-3-carbonitrile |
| | 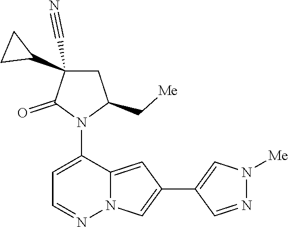 [SMILES: CC[C@@H]1C[C@](C#N)(C2CC2)C(=O)N1c1ccnn2cc(-c3cnn(C)c3)cc12] |
| | Bromide: (3R,5S)-1-(6-bromopyrrolo[1,2-b]pyridazin-4-yl)-3-cyclopropyl- |
| | 5-ethyl-2-oxopyrrolidine-3-carbonitrile (Preparation 91) |
| | Boronate: 1-methyl-4-(4,4,5,5-tetramethyl-1,3,2-dioxaborolan-2-yl)-1H- |
| | pyrazole |
| | HPLC-3 (gradient 30-50%); Green solid (13 mg, 32%). |
| | LCMS m/z = 375.1 [M + H]⁺; ¹H NMR (400 MHz, CDCl₃) δ: 8.02 (d, 1H), |
| | 7.91 (d, 1H), 7.73 (s, 1H), 7.61 (s, 1H), 6.52-6.48 (m, 2H), 4.40-4.33 (m, |
| | 1H), 3.95 (s, 3H), 2.87-2.82 (m, 1H), 2.01-1.99 (m, 1H), 1.79-1.75 (m, 1H), |
| | 1.40-1.37 (m, 2H), 0.89-0.74 (m, 7H). |

### Example 34

### (3R,4S)-3-cyclopropyl-4-methyl-1-(6-(1-methyl-1H-pyrazol-4-yl)pyrrolo[1,2-b]pyridazin-4-yl)-2-oxopyrrolidine-3-carbonitrile

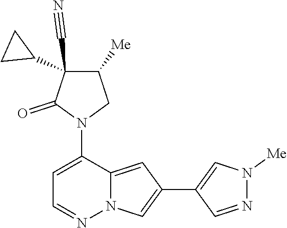 [SMILES: C[C@@H]1CN(c2ccnn3cc(-c4cnn(C)c4)cc23)C(=O)[C@]1(C#N)C1CC1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 3.2 g, 8.91 mmol) in dioxane (3 mL) and H₂O (0.3 mL) was added 1-methyl-4-(4,4,5,5-tetramethyl-1,3,2-dioxaborolan-2-yl)-1H-pyrazole (2.8 g, 13.4 mmol), KF (1.6 g, 26.7 mmol) and Pd(dppf)Cl₂ (652 mg, 0.891 mmol) and the rection stirred at 70° C. for 1 h. The mixture was concentrated in vacuo and purified by prep-HPLC-3 (gradient 32-62%) to afford (3R,4S)-3-cyclopropyl-4-methyl-1-(6-(1-methyl-1H-pyrazol-4-yl)pyrrolo[1,2-b]pyridazin-4-yl)-2-oxopyrrolidine-3-carbonitrile as a green solid (1.1 g, 34%). LCMS m/z=361.1 [M+H]⁺; ¹H NMR (500 MHz, CDCl₃) δ: 7.97 (d, 1H), 7.89 (s, 1H), 7.73 (s, 1H), 7.62 (s, 1H), 6.84 (d, 1H), 6.59 (s, 1H), 4.13-4.09 (m, 1H), 3.95 (s, 3H), 3.88-3.84 (m, 1H), 3.11-3.06 (m, 1H), 1.43 (d, 3H), 1.21-1.16 (m, 1H), 0.82-0.76 (m, 4H).

### Example 35

### (3R,4S)-3-cyclopropyl-1-(6-(1-(difluoromethyl)-1H-pyrazol-4-yl)pyrrolo[1,2-b]pyridazin-4-yl)-4-methyl-2-oxopyrrolidine-3-carbonitrile

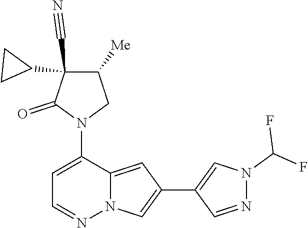 [SMILES: C[C@@H]1CN(c2ccnn3cc(-c4cnn(C(F)F)c4)cc23)C(=O)[C@]1(C#N)C1CC1]

(3R,4S)-3-Cyclopropyl-1-(6-(1-(difluoromethyl)-1H-pyrazol-4-yl)pyrrolo[1,2-b]pyridazin-4-yl)-4-methyl-2-oxopyrrolidine-3-carbonitrile was prepared as a white solid (54.1 mg, 70%) from (3R,4S)-1-(6-bromopyrrolo[1,2-b]pyridazin-4-yl)-3-cyclopropyl-4-methyl-2-oxopyrrolidine-3-carbonitrile (Preparation 68) and 1-(difluoromethyl)-4-(4,4,5,5-tetramethyl-1,3,2-dioxaborolan-2-yl)-1H-pyrazole, following the procedure described in Example 35. LCMS m/z=397.1 [M+H]⁺; ¹H NMR (400 MHz, CDCl₃) δ: 8.04-8.01 (m, 2H), 7.95 (s, 1H), 7.90 (s, 1H), 7.22 (d, 1H), 6.87 (d, 1H), 6.45 (s, 1H), 4.14-4.10 (m, 1H), 3.87 (t, 1H), 3.13-3.07 (m, 1H), 1.44 (d, 3H), 1.20-1.15 (m, 1H), 0.83-0.78 (m, 4H).

### Example 36 and 37

### (R)-3-cyclopropyl-1-(6-(1-methyl-1H-pyrazol-4-yl)pyrrolo[1,2-b]pyridazin-4-yl)pyrrolidin-2-one and (S)-3-cyclopropyl-1-(6-(1-methyl-1H-pyrazol-4-yl)pyrrolo[1,2-b]pyridazin-4-yl)pyrrolidin-2-one

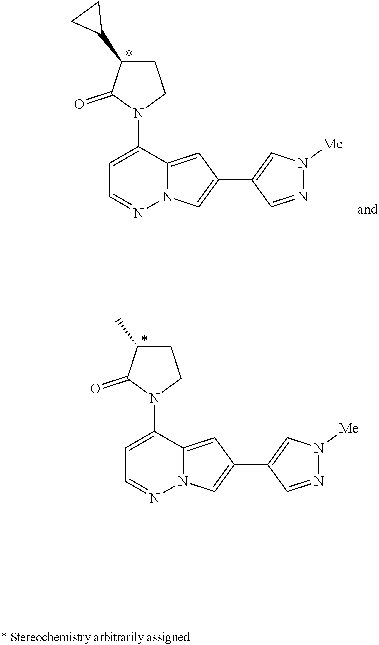 [SMILES: Cn1cc(-c2cc3c(N4CC[C@H](C5CC5)C4=O)ccnn3c2)cn1 ; C[C@@H]1CCN(c2ccnn3cc(-c4cnn(C)c4)cc23)C1=O]

To a solution of 1-(6-bromopyrrolo[1,2-b]pyridazin-4-yl)-3-cyclopropylpyrrolidin-2-one (Preparation 71, 220 mg, 0.687 mmol) and 1-methyl-4-(4,4,5,5-tetramethyl-1,3,2-dioxaborolan-2-yl)-1H-pyrazole (214 mg, 1.03 mmol) in dioxane (5 mL) and water (0.5 mL) was added Pd(dppf)Cl₂ (50.3 mg, 0.069 mmol) and K₂CO₃ (285 mg, 2.06 mmol) and the reaction stirred at 70° C. for 4 h. The mixture was concentrated in vacuo and purified by prep-HPLC-3 (gradient 28-58%) to give 3-cyclopropyl-1-(6-(1-methyl-1H-pyrazol-4-yl)pyrrolo[1,2-b]pyridazin-4-yl)pyrrolidin-2-one as a green solid (192 mg, 87%). This was further purified by SFC (DAICEL CHIRALCEL OJ-H, 250×30 mm, 5 μm; 30% (0.1% NH₄OH/EtOH) in CO₂) to give the title compounds.

*Peak 1, Example 36: (R)-3-cyclopropyl-1-(6-(1-methyl-1H-pyrazol-4-yl)pyrrolo[1,2-b]pyridazin-4-yl)pyrrolidin-2-one as a yellow solid (66.9 mg, 35%). LCMS m/z=322.2 [M+H]⁺; ¹H NMR (500 MHz, CDCl₃) δ: 7.95 (d, 1H), 7.85 (s, 1H), 7.72 (s, 1H), 7.59 (s, 1H), 6.85 (d, 1H), 6.64 (s, 1H), 4.00-4.08 (m, 2H), 3.94 (s, 3H), 2.37-2.36 (m, 1H), 2.27-2.26 (m, 1H), 2.02-1.98 (m, 1H), 1.09-1.07 (m, 1H), 0.75-0.69 (m, 1H), 0.56-0.51 (m, 2H), 0.33-0.31 (m, 1H).

*Peak 2, Example 37: (S)-3-cyclopropyl-1-(6-(1-methyl-1H-pyrazol-4-yl)pyrrolo[1,2-b]pyridazin-4-yl)pyrrolidin-2-one as a green solid (56.3 mg, 29%). LCMS m/z=322.1 [M+H]⁺; ¹H NMR (500 MHz, CDCl₃) δ: 7.94 (d, 1H), 7.84 (d, 1H), 7.72 (s, 1H), 7.59 (s, 1H), 6.85 (d, 1H), 6.63 (d, 1H), 4.06-4.01 (m, 2H), 3.93 (s, 3H), 2.37-2.35 (m, 1H), 2.28-2.25 (m, 1H), 2.01-1.99 (m, 1H), 1.09-1.07 (m, 1H), 0.75-0.69 (m, 1H), 0.55-0.49 (m, 2H), 0.33-0.31 (m, 1H).

### Example 38 and 39

### (S)-3-methyl-1-(6-(1-methyl-1H-pyrazol-4-yl)pyrrolo[1,2-b]pyridazin-4-yl)-2-oxopyrrolidine-3-carbonitrile and (R)-3-methyl-1-(6-(1-methyl-1H-pyrazol-4-yl)pyrrolo[1,2-b]pyridazin-4-yl)-2-oxopyrrolidine-3-carbonitrile

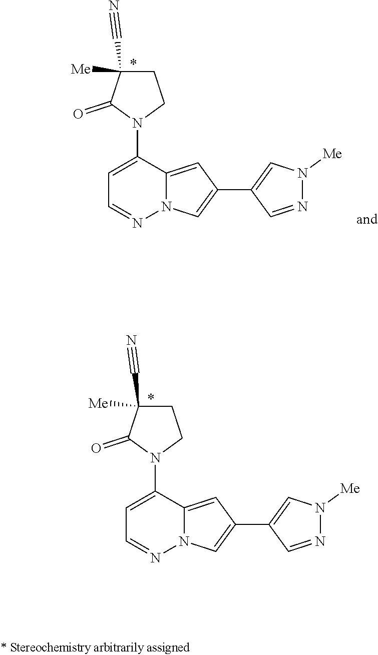 [SMILES: Cn1cc(-c2cc3c(N4CC[C@@](C)(C#N)C4=O)ccnn3c2)cn1 ; Cn1cc(-c2cc3c(N4CC[C@](C)(C#N)C4=O)ccnn3c2)cn1]

To a solution of 1-(6-bromopyrrolo[1,2-b]pyridazin-4-yl)-3-methyl-2-oxopyrrolidine-3-carbonitrile (Preparation 72, 100.0 mg, 0.313 mmol) and 1-methyl-4-(4,4,5,5-tetramethyl-1,3,2-dioxaborolan-2-yl)-1H-pyrazole (130.4 mg, 0.627 mmol) in dioxane (10.0 mL) and H₂O (1.0 mL) was added KF (54.6 mg, 0.94 mmol) and Pd(dppf)Cl₂ (22.9 mg, 0.031 mmol) and the reaction stirred at 70° C. for 4 h under N₂. The cooled mixture was concentrated in vacuo and the residue purified by prep-HPLC-3 (gradient 30-60%) to give 3-methyl-1-(6-(1-methyl-1H-pyrazol-4-yl)pyrrolo[1,2-b]pyridazin-4-yl)-2-oxopyrrolidine-3-carbonitrile as a green solid (80 mg, 80%). This was further purified by SFC (DAICEL CHIRALPAK AD, 250×30 mm, 10 μm; 55% EtOH in CO₂) to give the title compounds.

*Peak 1, Example 38: (S)-3-methyl-1-(6-(1-methyl-1H-pyrazol-4-yl)pyrrolo[1,2-b]pyridazin-4-yl)-2-oxopyrrolidine-3-carbonitrile as a green solid (30.5 mg, 38%). LCMS m/z=321.2 [M+H]⁺; ¹H NMR (400 MHz, CDCl₃) δ: 7.99 (d, 1H), 7.90 (d, 1H), 7.73 (s, 1H), 7.61 (s, 1H), 6.78 (d, 1H), 6.59 (s, 1H), 4.25-4.20 (m, 1H), 4.11-4.08 (m, 1H), 3.95 (s, 3H), 2.85-2.79 (m, 1H), 2.37-2.31 (m, 1H), 1.76 (s, 3H).

*Peak 2, Example 39: (R)-3-methyl-1-(6-(1-methyl-1H-pyrazol-4-yl)pyrrolo[1,2-b]pyridazin-4-yl)-2-oxopyrrolidine-3-carbonitrile (33.3 mg, 42%). LCMS m/z=321.2 [M+H]⁺; ¹H NMR (400 MHz, CDCl₃) δ: 7.99 (d, 1H), 7.90 (s, 1H), 7.73 (s, 1H), 7.61 (s, 1H), 6.78 (d, 1H), 6.59 (d, 1H), 4.23-4.18 (m, 1H), 4.10-4.07 (m, 1H), 3.95 (s, 3H), 2.85-2.79 (m, 1H), 2.37-2.31 (m, 1H), 1.76 (s, 3H).

### Example 40 and 41

### (S)-3-(methoxymethyl)-1-(6-(1-methyl-1H-pyrazol-4-yl)pyrrolo[1,2-b]pyridazin-4-yl)-2-oxopyrrolidine-3-carbonitrile and (R)-3-(methoxymethyl)-1-(6-(1-methyl-1H-pyrazol-4-yl)pyrrolo[1,2-b]pyridazin-4-yl)-2-oxopyrrolidine-3-carbonitrile

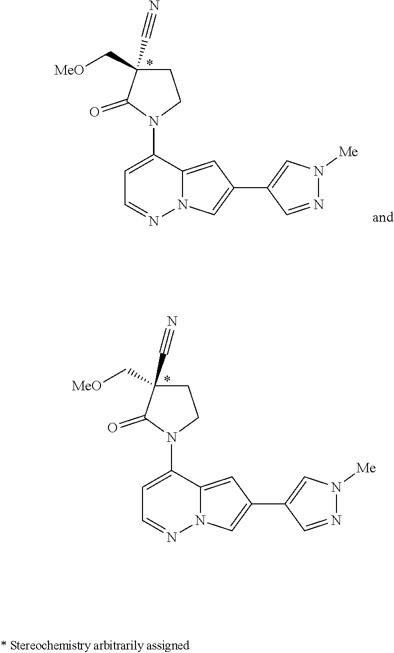 [SMILES: COC[C@@]1(C#N)CCN(c2ccnn3cc(-c4cnn(C)c4)cc23)C1=O ; COC[C@]1(C#N)CCN(c2ccnn3cc(-c4cnn(C)c4)cc23)C1=O]

3-(Methoxymethyl)-1-(6-(1-methyl-1H-pyrazol-4-yl)pyrrolo[1,2-b]pyridazin-4-yl)-2-oxopyrrolidine-3-carbonitrile was obtained as a green solid from 1-(6-bromopyrrolo[1,2-b]pyridazin-4-yl)-3-(methoxymethyl)-2-oxopyrrolidine-3-carbonitrile (Preparation 67) and 1-methyl-4-(4,4,5,5-tetramethyl-1,3,2-dioxaborolan-2-yl)-1H-pyrazole, following the procedure described in Example 36 and 37. The product was further purified by SFC (DAICEL CHIRALPAK AD, 250×30 mm, 10 μm); 45% (0.1% NH₄OH/EtOH in CO₂) to give the title compounds.

*Peak 1, Example 40: (S)-3-(methoxymethyl)-1-(6-(1-methyl-1H-pyrazol-4-yl)pyrrolo[1,2-b]pyridazin-4-yl)-2-oxopyrrolidine-3-carbonitrile (green solid, 36.4 mg, 36.4%). LCMS m/z=351.1 [M+H]⁺; ¹H NMR (500 MHz, CDCl₃) δ: 7.99 (d, 1H), 7.90 (d, 1H), 7.73 (s, 1H), 7.61 (s, 1H), 6.73 (d, 1H), 6.58 (s, 1H), 4.15-4.08 (m, 2H), 3.98-3.96 (m, 4H), 3.85-3.83 (m, 1H), 3.48 (s, 3H), 2.74-2.71 (m, 2H).

*Peak 2, Example 41: (R)-3-(methoxymethyl)-1-(6-(1-methyl-1H-pyrazol-4-yl)pyrrolo[1,2-b]pyridazin-4-yl)-2-oxopyrrolidine-3-carbonitrile (green solid, 29.6 mg, 29.60% yield). LCMS m/z=351.1 [M+H]⁺; ¹H NMR (500 MHz, CDCl₃) δ: 7.99 (d, 1H), 7.90 (d, 1H), 7.73 (s, 1H), 7.61 (s, 1H), 6.73 (d, 1H), 6.58 (d, 1H), 4.13-4.07 (m, 2H), 3.98-3.96 (m, 4H), 3.85-3.83 (m, 1H), 3.48 (s, 3H), 2.73-2.71 (m, 2H).

### Example 42 and 43

### (R)-3-ethyl-1-(6-(1-methyl-1H-pyrazol-4-yl)pyrrolo[1,2-b]pyridazin-4-yl)-2-oxopyrrolidine-3-carbonitrile and (S)-3-ethyl-1-(6-(1-methyl-1H-pyrazol-4-yl)pyrrolo[1,2-b]pyridazin-4-yl)-2-oxopyrrolidine-3-carbonitrile

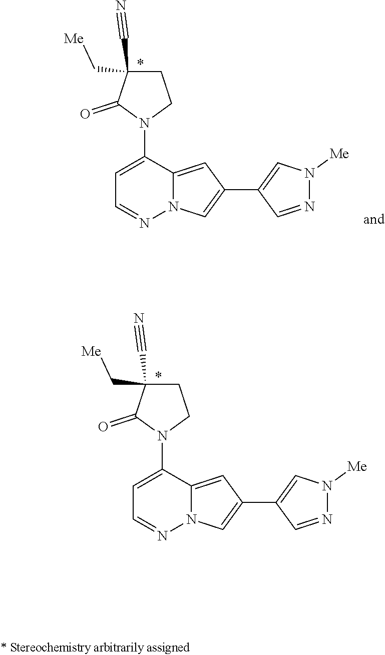 [SMILES: CC[C@]1(C#N)CCN(c2ccnn3cc(-c4cnn(C)c4)cc23)C1=O ; CC[C@@]1(C#N)CCN(c2ccnn3cc(-c4cnn(C)c4)cc23)C1=O]

3-Ethyl-1-(6-(1-methyl-1H-pyrazol-4-yl)pyrrolo[1,2-b]pyridazin-4-yl)-2-oxopyrrolidine-3-carbonitrile was obtained as a yellow solid, 111 mg, 43.9% yield from 1-(6-bromopyrrolo[1,2-b]pyridazin-4-yl)-3-ethyl-2-oxopyrrolidine-3-carbonitrile (Preparation 73) and 1-methyl-4-(4,4,5,5-tetramethyl-1,3,2-dioxaborolan-2-yl)-1H-pyrazole, following a similar procedure to that described in Example 38 and 39, except HPLC-2 (gradient 30-60%) was used to purify the compound. The compound was further purified by SFC (CHIRALPAK AD, 250×30 mm, 10 μm; 55% (0.1% NH₄OH in EtOH) in CO₂) to afford the title compounds.

*Peak 1, Example 42: (R)-3-ethyl-1-(6-(1-methyl-1H-pyrazol-4-yl)pyrrolo[1,2-b]pyridazin-4-yl)-2-oxopyrrolidine-3-carbonitrile as a yellow solid (51 mg, 45%). LCMS m/z=335.1 [M+H]⁺; ¹H NMR (400 MHz, CDCl₃) δ: 7.97 (d, 1H), 7.88 (s, 1H), 7.71 (s, 1H), 7.59 (s, 1H), 6.76 (d, 1H), 6.57 (s, 1H), 4.20-4.15 (m, 1H), 4.08-4.05 (m, 1H), 3.93 (s, 3H), 2.74-2.72 (m, 1H), 2.36-2.21 (m, 2H), 1.92-1.86 (m, 1H), 1.23 (t, 3H).

*Peak 2, Example 43: (S)-3-ethyl-1-(6-(1-methyl-1H-pyrazol-4-yl)pyrrolo[1,2-b]pyridazin-4-yl)-2-oxopyrrolidine-3-carbonitrile as a yellow solid (52 mg, 46%). LCMS m/z=335.1 [M+H]⁺; ¹H NMR (400 MHz, CDCl₃) δ: 7.90 (d, 1H), 7.90 (s, 1H), 7.73 (s, 1H), 7.61 (s, 1H), 6.77 (d, 1H), 6.58 (s, 1H), 4.21-4.16 (m, 1H), 4.10-4.07 (m, 1H), 3.95 (s, 3H), 2.76-2.74 (m, 1H), 2.38-2.23 (m, 2H), 1.25 (t, 1H).

### Example 44 and 45

### (R)-1-(6-(1-methyl-1H-pyrazol-4-yl)pyrrolo[1,2-b]pyridazin-4-yl)-3-(oxetan-3-yl)-2-oxopyrrolidine-3-carbonitrile and (S)-1-(6-(1-methyl-1H-pyrazol-4-yl)pyrrolo[1,2-b]pyridazin-4-yl)-3-(oxetan-3-yl)-2-oxopyrrolidine-3-carbonitrile

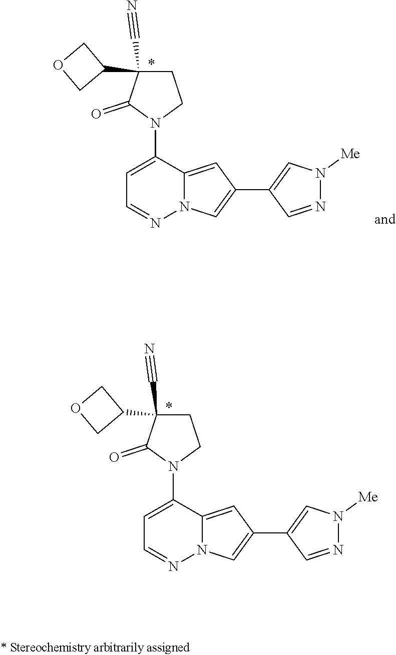 [SMILES: Cn1cc(-c2cc3c(N4CC[C@](C#N)(C5COC5)C4=O)ccnn3c2)cn1 ; Cn1cc(-c2cc3c(N4CC[C@@](C#N)(C5COC5)C4=O)ccnn3c2)cn1]

The title compounds were prepared from 1-methyl-4-(4,4,5,5-tetramethyl-1,3,2-dioxaborolan-2-yl)-1H-pyrazole and 1-(6-bromopyrrolo[1,2-b]pyridazin-4-yl)-3-(oxetan-3-yl)-2-oxopyrrolidine-3-carbonitrile (Preparation 75), following the procedure described in Example 38 and 39.

*Peak 1, Example 44: (R)-1-(6-(1-methyl-1H-pyrazol-4-yl)pyrrolo[1,2-b]pyridazin-4-yl)-3-(oxetan-3-yl)-2-oxopyrrolidine-3-carbonitrile (green solid, 65 mg, 36%) LCMS m/z=363.2 [M+H]⁺; ¹H NMR (500 MHz, CDCl₃) δ ppm: 7.99 (d, 1H), 7.91 (d, 1H), 7.73 (s, 1H), 7.61 (s, 1H), 6.73 (d, 1H), 6.56 (d, 1H), 4.98-4.95 (m, 2H), 4.94-4.90 (m, 1H), 4.75-4.73 (m, 1H), 4.29-4.26 (m, 1H), 4.13-4.11 (m, 1H), 3.96 (s, 3H), 3.65-3.61 (m, 1H), 2.81-2.78 (m, 1H), 2.48-2.42 (m, 1H).

*Peak 2, Example 45: (S)-1-(6-(1-methyl-1H-pyrazol-4-yl)pyrrolo[1,2-b]pyridazin-4-yl)-3-(oxetan-3-yl)-2-oxopyrrolidine-3-carbonitrile (green solid, 65 mg, 36%). LCMS m/z=363.1 [M+H]⁺; ¹H NMR (500 MHz, CDCl₃) δ: 7.99 (d, 1H), 7.91 (s, 1H), 7.73 (s, 1H), 7.61 (s, 1H), 6.73 (d, 1H), 6.56 (s, 1H), 4.98-4.95 (m, 2H), 4.94-4.90 (m, 1H), 4.75-4.73 (m, 1H), 4.29-4.26 (m, 1H), 4.13-4.11 (m, 1H), 3.96 (s, 3H), 3.65-3.61 (m, 1H), 2.83-2.78 (m, 1H), 2.47-2.44 (m, 1H).

### Example 46 and 47

### (S)-3-cyclobutyl-1-(6-(1-methyl-1H-pyrazol-4-yl)pyrrolo[1,2-b]pyridazin-4-yl)-2-oxopyrrolidine-3-carbonitrile and (R)-3-cyclobutyl-1-(6-(1-methyl-1H-pyrazol-4-yl)pyrrolo[1,2-b]pyridazin-4-yl)-2-oxopyrrolidine-3-carbonitrile

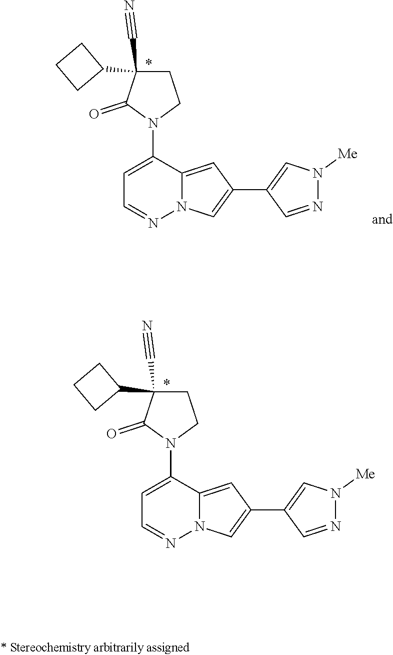 [SMILES: Cn1cc(-c2cc3c(N4CC[C@@](C#N)(C5CCC5)C4=O)ccnn3c2)cn1 ; Cn1cc(-c2cc3c(N4CC[C@](C#N)(C5CCC5)C4=O)ccnn3c2)cn1]

3-Cyclobutyl-1-(6-(1-methyl-1H-pyrazol-4-yl)pyrrolo[1,2-b]pyridazin-4-yl)-2-oxopyrrolidine-3-carbonitrile was prepared from 1-methyl-4-(4,4,5,5-tetramethyl-1,3,2-dioxaborolan-2-yl)-1H-pyrazole and 1-(6-bromopyrrolo[1,2-b]pyridazin-4-yl)-3-cyclobutyl-2-oxopyrrolidine-3-carbonitrile (Preparation 74), following a similar procedure to that described in Example 38 and 39. The compound was further purified by SFC (Daicel CHIRALPAK AD, 250×30 mm, 10 μm; 55% EtOH (0.1% NH₄OH) in CO₂) to afford the title compounds.

*Peak 1, Example 46: (S)-3-cyclobutyl-1-(6-(1-methyl-1H-pyrazol-4-yl)pyrrolo[1,2-b]pyridazin-4-yl)-2-oxopyrrolidine-3-carbonitrile (45.6 mg, as a green solid). LCMS m/z=361.1 [M+H]⁺; ¹H NMR (400 MHz, CDCl₃) δ: 7.96 (d, 1H), 7.88 (s, 1H), 7.72 (s, 1H), 7.60 (s, 1H), 6.74 (d, 1H), 6.57 (s, 1H), 4.12-4.04 (m, 2H), 3.94 (s, 3H), 2.92-2.89 (m, 1H), 2.72-2.62 (m, 1H), 2.33-2.16 (m, 5H), 2.00-1.95 (m, 2H).

*Peak 2, Example 47: (R)-3-cyclobutyl-1-(6-(1-methyl-1H-pyrazol-4-yl)pyrrolo[1,2-b]pyridazin-4-yl)-2-oxopyrrolidine-3-carbonitrile, 53.6 mg. LCMS m/z=361.1 [M+H]⁺; ¹H NMR (400 MHz, CDCl₃) δ: 7.97 (d, 1H), 7.89 (s, 1H), 7.72 (s, 1H), 7.61 (s, 1H), 6.75 (d, 1H), 6.57 (s, 1H), 4.12-4.05 (m, 2H), 3.95 (s, 3H), 2.93-2.90 (m, 1H), 2.68-2.66 (m, 1H), 2.34-2.19 (m, 5H), 1.99-1.96 (m, 2H).

### Example 48-98

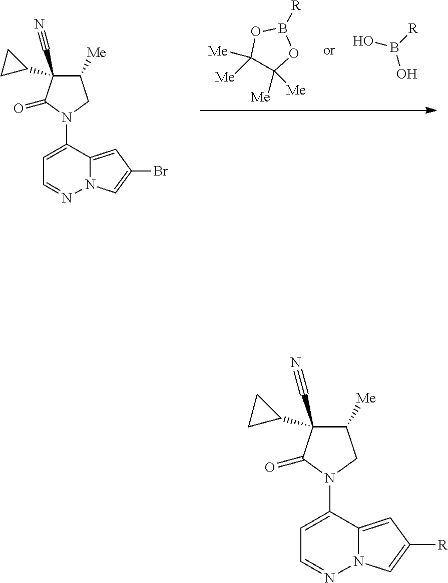

The title compounds were prepared using a one-step library protocol described below.

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 55 mg, 0.153 mmol, 1.00 eq) and the appropriate boronate ester or boronic acid (0.184 mmol, 1.20 eq) in dioxane (3 mL), H₂O (0.20 mL) was added K₃PO₄ (64.81 mg, 0.306 mmol, 2.00 eq) and Pd(dppf)Cl₂-DCM (6.53 mg, 0.008 mmol, 0.05 eq) under N₂. The mixture was stirred at 100° C. for 16 h. Water (5 mL) was added to the reaction mixture and extracted with EtOAc (3×5 mL). The combined organics were evaporated to dryness by speedvac and the residue purified by prep-HPLC-15 or 16 as note in the table, or (Xtimate C18, 150×25 mm, 5 Dim; H₂O (0.075% TFA)-MeCN); gradient optimised for each example to give the title compound. For some compounds the title compounds precipitated from the reaction mixtures. The solids were collected by filtration and washed with MeCN/H₂O (3×) and dried by lyophilisation. These compounds are noted as precipitate in the following table.

| Example | |
|---|---|
| No. | Name/Structure/Reactants/HPLC/Data |
| 48 | (3R,4S)-3-cyclopropyl-1-(6-(6-methoxypyridin-3-yl)pyrrolo[1,2-b]pyridazin-4- |
| | yl)-4-methyl-2-oxopyrrolidine-3-carbonitrile |
| | 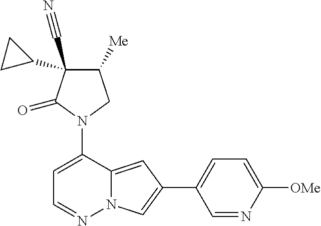 [SMILES: COc1ccc(-c2cc3c(N4C[C@@H](C)[C@@](C#N)(C5CC5)C4=O)ccnn3c2)cn1] |
| | Boronate/Boronic acid: (6-methoxypyridin-3-yl)boronic acid; prep-HPLC-16; |
| | LCMS m/z = 388.2 [M + H]⁺ |
| 49 | (3R,4S)-3-cyclopropyl-4-methyl-1-(6-(1-methyl-1H-pyrazol-5-yl)pyrrolo[1,2- |
| | b]pyridazin-4-yl)-2-oxopyrrolidine-3-carbonitrile |
| | 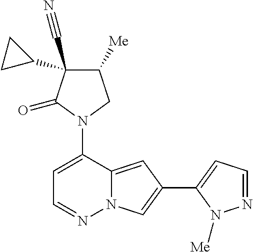 [SMILES: C[C@@H]1CN(c2ccnn3cc(-c4ccnn4C)cc23)C(=O)[C@]1(C#N)C1CC1] |
| | 16; LCMS m/z = 361.2 [M + H]⁺ |
| 50 | (3R,4S)-3-cyclopropyl-1-(6-(2-(difluoromethoxy)pyridin-3-y1)pyrrolo[1,2- |
| | b]pyridazin-4-yl)-4-methyl-2-oxopyrrolidine-3-carbonitrile |
| | 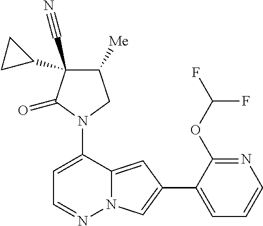 [SMILES: C[C@@H]1CN(c2ccnn3cc(-c4cccnc4OC(F)F)cc23)C(=O)[C@]1(C#N)C1CC1] |
| | Boronate/Boronic acid: (2-(difluoromethoxy)pyridin-3-yl)boronic acid; prep- |
| | HPLC-16; LCMS m/z = 424.2 [M + H]⁺ |
| 51 | (3R,4S)-3-cyclopropyl-1-(6-(imidazo[1,5-a]pyridin-6-yl)pyrrolo[1,2- |
| | b]pyridazin-4-yl)-4-methyl-2-oxopyrrolidine-3-carbonitrile |
| | 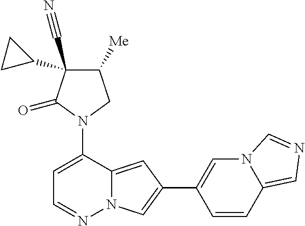 [SMILES: C[C@@H]1CN(c2ccnn3cc(-c4ccc5cncn5c4)cc23)C(=O)[C@]1(C#N)C1CC1] |
| | Boronate/Boronic acid: 6-(4,4,5,5-tetramethyl-1,3,2-dioxaborolan-2- |
| | yl)imidazo[1,5-a]pyridine; prep-HPLC-16; LCMS m/z = 397.1 [M + H]⁺ |
| 52 | (3R,4S)-3-cyclopropyl-4-methyl-1-(6-(6-(methylsulfonyl)pyridin-3- |
| | yl)pyrrolo[1,2-b]pyridazin-4-yl)-2-oxopyrrolidine-3-carbonitrile |
| | 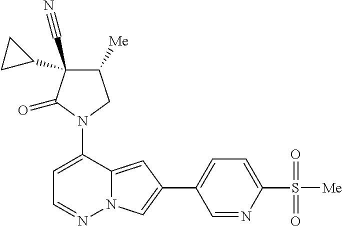 [SMILES: C[C@@H]1CN(c2ccnn3cc(-c4ccc(S(C)(=O)=O)nc4)cc23)C(=O)[C@]1(C#N)C1CC1] |
| | Boronate/Boronic acid: (6-(methylsulfonyl)pyridin-3-yl)boronic acid; prep- |
| | HPLC-16; LCMS m/z = 436.1 [M + H]⁺ |
| 53 | (3R,4S)-3-cyclopropyl-1-(6-(5-hydroxypyridin-3-yl)pyrrolo[1,2-b]pyridazin-4- |
| | yl)-4-methyl-2-oxopyrrolidine-3-carbonitrile |
| | 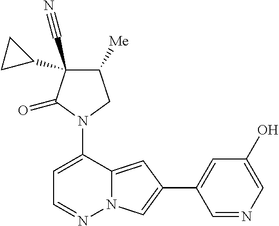 [SMILES: C[C@@H]1CN(c2ccnn3cc(-c4cncc(O)c4)cc23)C(=O)[C@]1(C#N)C1CC1] |
| | Boronate/Boronic acid: 5-(4,4,5,5-tetramethyl-1,3,2-dioxaborolan-2-yl)pyridin- |
| | 3-ol; prep-HPLC-16; LCMS m/z = 374.1 [M + H]⁺ |
| 54 | (3R,4S)-3-cyclopropyl-4-methyl-1-(6-(1-methyl-1H-indazol-4-yl)pyrrolo[1,2- |
| | b]pyridazin-4-yl)-2-oxopyrrolidine-3-carbonitrile |
| | 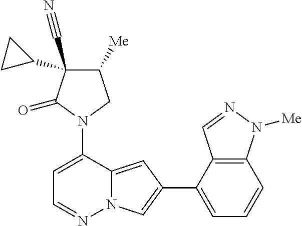 [SMILES: C[C@@H]1CN(c2ccnn3cc(-c4cccc5c4cnn5C)cc23)C(=O)[C@]1(C#N)C1CC1] |
| | Boronate/Boronic acid: (1-methyl-1H-indazol-4-yl)boronic acid; LCMS m/z = |
| | 411.2 [M + H]⁺ |
| 55 | (3R,4S)-3-cyclopropyl-4-methyl-1-(6-(1-methyl-1H-indazol-5-yl)pyrrolo[1,2- |
| | b]pyridazin-4-yl)-2-oxopyrrolidine-3-carbonitrile |
| | 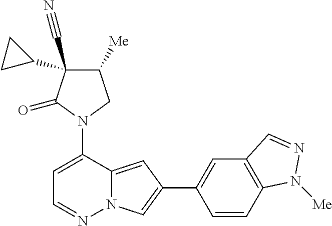 [SMILES: C[C@@H]1CN(c2ccnn3cc(-c4ccc5c(cnn5C)c4)cc23)C(=O)[C@]1(C#N)C1CC1] |
| | Boronate/Boronic acid: (1-methyl-1H-indazol-5-yl)boronic acid; Precipitate; |
| | LCMS m/z = 411.2 [M + H]⁺ |
| 56 | (3R,4S)-3-cyclopropyl-1-(6-(2-fluoropyridin-3-yl)pyrrolo[1,2-b]pyridazin-4- |
| | yl)-4-methyl-2-oxopyrrolidine-3-carbonitrile |
| | 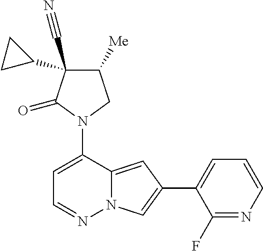 [SMILES: C[C@@H]1CN(c2ccnn3cc(-c4cccnc4F)cc23)C(=O)[C@]1(C#N)C1CC1] |
| | Boronate/Boronic acid: 2-fluoro-3-(4,4,5,5-tetramethyl-1,3,2-dioxaborolan-2- |
| | yl)pyridine; Precipitate; LCMS m/z = 376.1 [M + H]⁺ |
| 57 | (3R,4S)-1-(6-([1,2,4]triazolo[1,5-a]pyridin-6-yl)pyrrolo[1,2-b]pyridazin-4-yl)- |
| | 3-cyclopropyl-4-methyl-2-oxopyrrolidine-3-carbonitrile |
| | 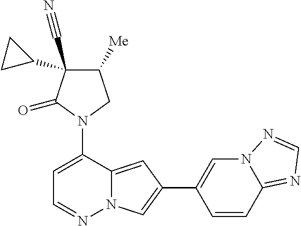 [SMILES: C[C@@H]1CN(c2ccnn3cc(-c4ccc5ncnn5c4)cc23)C(=O)[C@]1(C#N)C1CC1] |
| | Boronate/Boronic acid: 6-(4,4,5,5-tetramethyl-1,3,2-dioxaborolan-2-yl)- |
| | [1,2,4]triazolo[1,5-a]pyridine; Precipitate; LCMS m/z = 398.2 [M + H]⁺ |
| 58 | 5-(4-((3R,4S)-3-cyano-3-cyclopropyl-4-methyl-2-oxopyrrolidin-1- |
| | yl)pyrrolo[1,2-b]pyridazin-6-yl)-N-methylpicolinamide |
| | 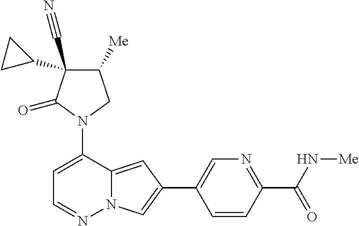 [SMILES: CNC(=O)c1ccc(-c2cc3c(N4C[C@@H](C)[C@@](C#N)(C5CC5)C4=O)ccnn3c2)cn1] |
| | Boronate/Boronic acid: N-methyl-5-(4,4,5,5-tetramethyl-1,3,2-dioxaborolan-2- |
| | yl)picolinamide; Precipitate; LCMS m/z = 415.2 [M + H]⁺ |
| 59 | (3R,4S)-1-(6-(1H-pyrazol-4-yl)pyrrolo[1,2-b]pyridazin-4-yl)-3-cyclopropyl-4- |
| | methyl-2-oxopyrrolidine-3-carbonitrile trifluoroacetate |
| | 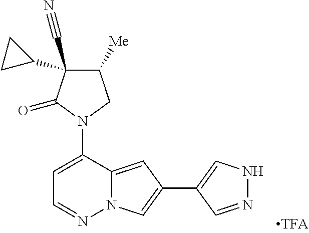 [SMILES: C[C@@H]1CN(c2ccnn3cc(-c4cn[nH]c4)cc23)C(=O)[C@]1(C#N)C1CC1] |
| | Boronate/Boronic acid: 4-(4,4,5,5-tetramethyl-1,3,2-dioxaborolan-2-yl)-1H- |
| | pyrazole; prep-HPLC-15; LCMS m/z = 347.1 [M + H]⁺ |
| 60 | (3R,4S)-3-cyclopropyl-4-methyl-2-oxo-1-(6-(pyridin-3-yl)pyrrolo[1,2- |
| | b]pyridazin-4-yl)pyrrolidine-3-carbonitrile |
| | 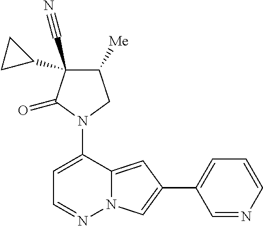 [SMILES: C[C@@H]1CN(c2ccnn3cc(-c4cccnc4)cc23)C(=O)[C@]1(C#N)C1CC1] |
| | Boronate/Boronic acid: 3-(4,4,5,5-tetramethyl-1,3,2-dioxaborolan-2- |
| | yl)pyridine; prep-HPLC-16; LCMS m/z = 358.2 [M + H]⁺ |
| 61 | N-(4-(4-((3R,4S)-3-cyano-3-cyclopropyl-4-methyl-2-oxopyrrolidin-1- |
| | yl)pyrrolo[1,2-b]pyridazin-6-yl)pyridin-2-yl)acetamide |
| | 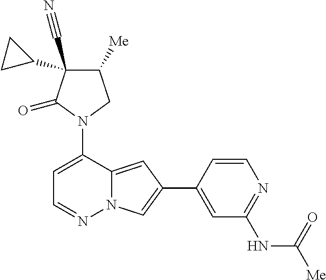 [SMILES: CC(=O)Nc1cc(-c2cc3c(N4C[C@@H](C)[C@@](C#N)(C5CC5)C4=O)ccnn3c2)ccn1] |
| | Boronate/Boronic acid: N-(4-(4,4,5,5-tetramethyl-1,3,2-dioxaborolan-2- |
| | yl)pyridin-2-yl)acetamide; prep-HPLC-16; LCMS m/z = 415.2 [M + H]⁺ |
| 62 | (3R,4S)-3-cyclopropyl-1-(6-(6-(dimethylamino)pyridin-3-yl)pyrrolo[1,2- |
| | b]pyridazin-4-yl)-4-methyl-2-oxopyrrolidine-3-carbonitrile |
| | 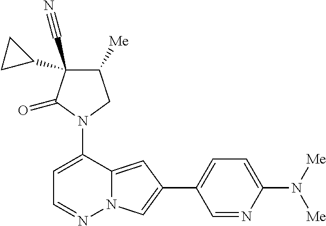 [SMILES: C[C@@H]1CN(c2ccnn3cc(-c4ccc(N(C)C)nc4)cc23)C(=O)[C@]1(C#N)C1CC1] |
| | Boronate/Boronic acid: (6-(dimethylamino)pyridin-3-yl)boronic acid; |
| | Precipitate; LCMS m/z = 401.2 [M + H]⁺ |
| 63 | (3R,4S)-3-cyclopropyl-4-methyl-1-(6-(1-methyl-1H-pyrazol-3-yl)pyrrolo[1,2- |
| | b]pyridazin-4-yl)-2-oxopyrrolidine-3-carbonitrile |
| | 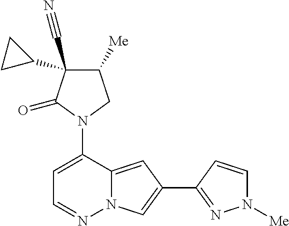 [SMILES: C[C@@H]1CN(c2ccnn3cc(-c4ccn(C)n4)cc23)C(=O)[C@]1(C#N)C1CC1] |
| | Boronate/Boronic acid: 1-methyl-3-(4,4,5,5-tetramethyl-1,3,2-dioxaborolan-2- |
| | yl)-1H-pyrazole; prep-HPLC-16; LCMS m/z = 361.2 [M + H]⁺ |
| 64 | (3R,4S)-1-(6-(1H-pyrrolo[2,3-b]pyridin-5-yl)pyrrolo[1,2-b]pyridazin-4-yl)-3- |
| | cyclopropyl-4-methyl-2-oxopyrrolidine-3-carbonitrile |
| | 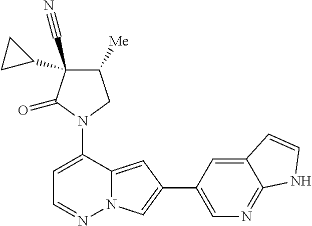 [SMILES: C[C@@H]1CN(c2ccnn3cc(-c4cnc5[nH]ccc5c4)cc23)C(=O)[C@]1(C#N)C1CC1] |
| | Boronate/Boronic acid: (1H-pyrrolo[2,3-b]pyridin-5-yl)boronic acid; prep- |
| | HPLC-16; LCMS m/z = 397.2 [M + H]⁺ |
| 65 | (3R,4S)-3-cyclopropyl-1-(6-(2-methoxypyridin-4-yl)pyrrolo[1,2-b]pyridazin-4- |
| | yl)-4-methyl-2-oxopyrrolidine-3-carbonitrile |
| | 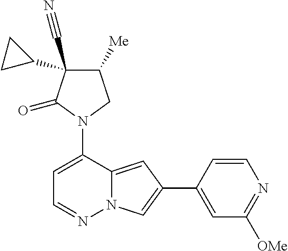 [SMILES: COc1cc(-c2cc3c(N4C[C@@H](C)[C@@](C#N)(C5CC5)C4=O)ccnn3c2)ccn1] |
| | Boronate/Boronic acid: (2-methoxypyridin-4-yl)boronic acid; prep-HPLC-16; |
| | LCMS m/z = 388.2 [M + H]⁺ |
| 66 | (3R,4S)-1-(6-(1H-pyrazol-3-yl)pyrrolo[1,2-b]pyridazin-4-yl)-3-cyclopropyl-4- |
| | methyl-2-oxopyrrolidine-3-carbonitrile trifluoroacetate |
| | 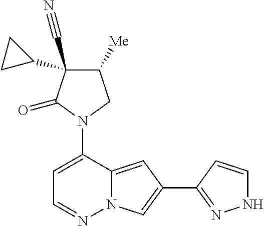 [SMILES: C[C@@H]1CN(c2ccnn3cc(-c4cc[nH]n4)cc23)C(=O)[C@]1(C#N)C1CC1] |
| | Boronate/Boronic acid: 3-(4,4,5,5-tetramethyl-1,3,2-dioxaborolan-2-yl)-1H- |
| | pyrazole; prep-HPLC-15; LCMS m/z = 347.2 [M + H]⁺ |
| 67 | (3R,4S)-3-cyclopropyl-4-methyl-1-(6-(6-methylpyridin-3-yl)pyrrolo[1,2- |
| | b]pyridazin-4-yl)-2-oxopyrrolidine-3-carbonitrile |
| | 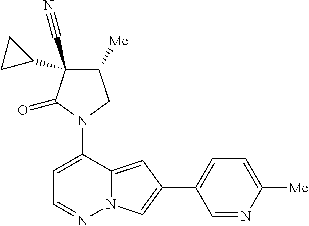 [SMILES: Cc1ccc(-c2cc3c(N4C[C@@H](C)[C@@](C#N)(C5CC5)C4=O)ccnn3c2)cn1] |
| | Boronate/Boronic acid: (6-methylpyridin-3-yl)boronic acid; prep-HPLC-16; |
| | LCMS m/z = 372.2 [M + H]⁺ |
| 68 | (3R,4S)-3-cyclopropyl-4-methyl-1-(6-(1-methyl-1H-indazol-6-yl)pyrrolo[1,2- |
| | b]pyridazin-4-yl)-2-oxopyrrolidine-3-carbonitrile |
| | 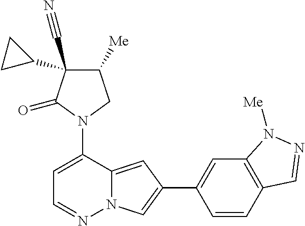 [SMILES: C[C@@H]1CN(c2ccnn3cc(-c4ccc5cnn(C)c5c4)cc23)C(=O)[C@]1(C#N)C1CC1] |
| | Boronate/Boronic acid: 1-methyl-6-(4,4,5,5-tetramethyl-1,3,2-dioxaborolan-2- |
| | yl)-1H-indazole; prep-HPLC-16; LCMS m/z = 411.3 [M + H]⁺ |
| 69 | (3R,4S)-3-cyclopropyl-4-methyl-2-oxo-1-(6-(2-(trifluoromethyl)pyridin-4- |
| | yl)pyrrolo[1,2-b]pyridazin-4-yl)pyrrolidine-3-carbonitrile |
| | 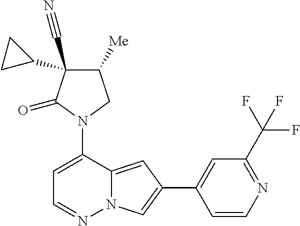 [SMILES: C[C@@H]1CN(c2ccnn3cc(-c4ccnc(C(F)(F)F)c4)cc23)C(=O)[C@]1(C#N)C1CC1] |
| | Boronate/Boronic acid: 4-(4,4,5,5-tetramethyl-1,3,2-dioxaborolan-2-yl)-2- |
| | (trifluoromethyl)pyridine; prep-HPLC-16; LCMS m/z = 426.2 [M + H]⁺ |
| 70 | (3R,4S)-3-cyclopropyl-4-methyl-2-oxo-1-(6-(5-(trifluoromethyl)pyridin-3- |
| | yl)pyrrolo[1,2-b]pyridazin-4-yl)pyrrolidine-3-carbonitrile |
| | 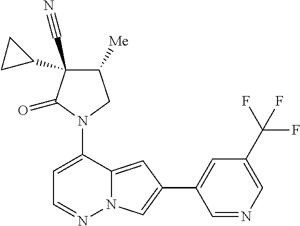 [SMILES: C[C@@H]1CN(c2ccnn3cc(-c4cncc(C(F)(F)F)c4)cc23)C(=O)[C@]1(C#N)C1CC1] |
| | Boronate/Boronic acid: 3-(4,4,5,5-tetramethyl-1,3,2-dioxaborolan-2-yl)-5- |
| | (trifluoromethyl)pyridine; prep-HPLC-16; LCMS m/z = 426.1 [M + H]⁺ |
| 71 | (3R,4S)-3-cyclopropyl-4-methyl-1-(6-(5-methylpyridin-3-yl)pyrrolo[1,2- |
| | b]pyridazin-4-yl)-2-oxopyrrolidine-3-carbonitrile |
| | 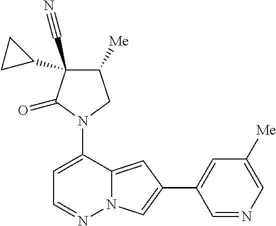 [SMILES: Cc1cncc(-c2cc3c(N4C[C@@H](C)[C@@](C#N)(C5CC5)C4=O)ccnn3c2)c1] |
| | Boronate/Boronic acid: (5-methylpyridin-3-yl)boronic acid; prep-HPLC-16; |
| | LCMS m/z = 372.2 [M + H]⁺ |
| 72 | (3R,4S)-1-(6-(1H-pyrrolo[2,3-b]pyridin-3-yl)pyrrolo[1,2-b]pyridazin-4-yl)-3- |
| | cyclopropyl-4-methyl-2-oxopyrrolidine-3-carbonitrile |
| | 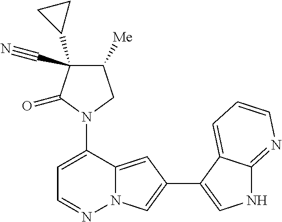 [SMILES: C[C@@H]1CN(c2ccnn3cc(-c4c[nH]c5ncccc45)cc23)C(=O)[C@]1(C#N)C1CC1] |
| | Boronate/Boronic acid: 3-(4,4,5,5-tetramethyl-1,3,2-dioxaborolan-2-yl)-1H- |
| | pyrrolo[2,3-b]pyridine; prep-HPLC-16; LCMS m/z = 397.2 [M + H]⁺ |
| 73 | (3R,4S)-3-cyclopropyl-1-(6-(2-fluoropyridin-4-yl)pyrrolo[1,2-b]pyridazin-4- |
| | yl)-4-methyl-2-oxopyrrolidine-3-carbonitrile |
| | 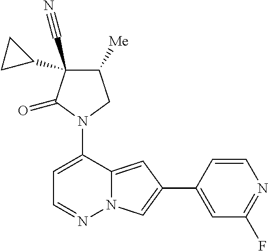 [SMILES: C[C@@H]1CN(c2ccnn3cc(-c4ccnc(F)c4)cc23)C(=O)[C@]1(C#N)C1CC1] |
| | Boronate/Boronic acid: (2-fluoropyridin-4-yl)boronic acid; prep-HPLC-16; |
| | LCMS m/z = 376.1 [M + H]⁺ |
| 74 | (3R,4S)-3-cyclopropyl-4-methyl-2-oxo-1-(6-(6-(trifluoromethyl)pyridin-3- |
| | yl)pyrrolo[1,2-b]pyridazin-4-yl)pyrrolidine-3-carbonitrile |
| | 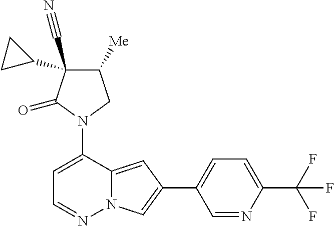 [SMILES: C[C@@H]1CN(c2ccnn3cc(-c4ccc(C(F)(F)F)nc4)cc23)C(=O)[C@]1(C#N)C1CC1] |
| | Boronate/Boronic acid: (6-(trifluoromethyl)pyridin-3-yl)boronic acid; prep- |
| | HPLC-16; LCMS m/z = 426.2 [M + H]⁺ |
| 75 | (3R,4S)-1-(6-(1H-pyrazolo[3,4-b]pyridin-5-yl)pyrrolo[1,2-b]pyridazin-4-yl)-3- |
| | cyclopropyl-4-methyl-2-oxopyrrolidine-3-carbonitrile |
| | 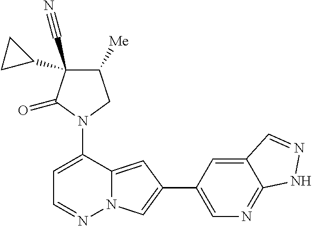 [SMILES: C[C@@H]1CN(c2ccnn3cc(-c4cnc5[nH]ncc5c4)cc23)C(=O)[C@]1(C#N)C1CC1] |
| | Boronate/Boronic acid: 5-(4,4,5,5-tetramethyl-1,3,2-dioxaborolan-2-yl)-1H- |
| | pyrazolo[3,4-b]pyridine; prep-HPLC-16; LCMS m/z = 398.2 [M + H]⁺ |
| 76 | (3R,4S)-3-cyclopropyl-1-(6-(2-methoxypyrimidin-5-yl)pyrrolo[1,2- |
| | b]pyridazin-4-yl)-4-methyl-2-oxopyrrolidine-3-carbonitrile |
| | 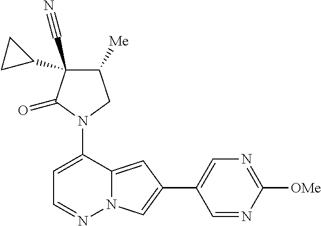 [SMILES: COc1ncc(-c2cc3c(N4C[C@@H](C)[C@@](C#N)(C5CC5)C4=O)ccnn3c2)cn1] |
| | Boronate/Boronic acid: (2-methoxypyrimidin-5-yl)boronic acid; prep-HPLC- |
| | 16; LCMS m/z = 389.2 [M + H]⁺ |
| 77 | (3R,4S)-3-cyclopropyl-4-methyl-1-(6-(2-methylpyridin-4-yl)pyrrolo[1,2- |
| | b]pyridazin-4-yl)-2-oxopyrrolidine-3-carbonitrile |
| | 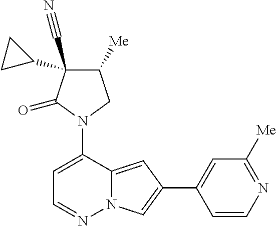 [SMILES: Cc1cc(-c2cc3c(N4C[C@@H](C)[C@@](C#N)(C5CC5)C4=O)ccnn3c2)ccn1] |
| | Boronate/Boronic acid: (2-methylpyridin-4-yl)boronic acid; prep-HPLC-16; |
| | LCMS m/z = 372.2 [M + H]⁺ |
| 78 | (3R,4S)-3-cyclopropyl-4-methyl-1-(6-(1-methyl-1H-indazol-7-yl)pyrrolo[1,2- |
| | b]pyridazin-4-yl)-2-oxopyrrolidine-3-carbonitrile |
| | 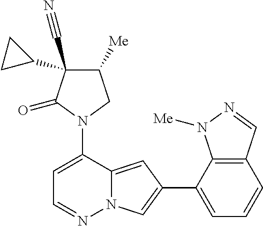 [SMILES: C[C@@H]1CN(c2ccnn3cc(-c4cccc5cnn(C)c45)cc23)C(=O)[C@]1(C#N)C1CC1] |
| | Boronate/Boronic acid: (1-methyl-1H-indazol-7-yl)boronic acid; prep-HPLC- |
| | 16; LCMS m/z = 411.2 [M + H]⁺ |
| 79 | (3R,4S)-3-cyclopropyl-4-methyl-1-(6-(1-methyl-2-oxo-1,2-dihydropyridin-4- |
| | yl)pyrrolo[1,2-b]pyridazin-4-yl)-2-oxopyrrolidine-3-carbonitrile |
| | 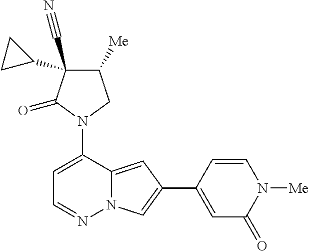 [SMILES: C[C@@H]1CN(c2ccnn3cc(-c4ccn(C)c(=O)c4)cc23)C(=O)[C@]1(C#N)C1CC1] |
| | Boronate/Boronic acid: 1-methyl-4-(4,4,5,5-tetramethyl-1,3,2-dioxaborolan-2- |
| | yl)pyridin-2(1H)-one; prep-HPLC-16; LCMS m/z = 388.2 [M + H]⁺ |
| 80 | (3R,4S)-3-cyclopropyl-4-methyl-1-(6-(2-methylpyrimidin-5-yl)pyrrolo[1,2- |
| | b]pyridazin-4-yl)-2-oxopyrrolidine-3-carbonitrile |
| | 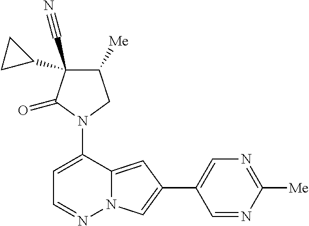 [SMILES: Cc1ncc(-c2cc3c(N4C[C@@H](C)[C@@](C#N)(C5CC5)C4=O)ccnn3c2)cn1] |
| | Boronate/Boronic acid: (2-methylpyrimidin-5-yl)boronic acid; prep-HPLC-16; |
| | LCMS m/z = 373.2 [M + H]⁺ |
| 81 | (3R,4S)-3-cyclopropyl-1-(6-(6-fluoropyridin-3-yl)pyrrolo[1,2-b]pyridazin-4- |
| | yl)-4-methyl-2-oxopyrrolidine-3-carbonitrile |
| | 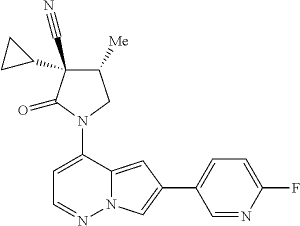 [SMILES: C[C@@H]1CN(c2ccnn3cc(-c4ccc(F)nc4)cc23)C(=O)[C@]1(C#N)C1CC1] |
| | Boronate/Boronic acid: (6-fluoropyridin-3-yl)boronic acid; prep-HPLC-16; |
| | LCMS m/z = 376.1 [M + H]⁺ |
| 82 | (3R,4S)-3-cyclopropyl-1-(6-(5-methoxypyridin-3-yl)pyrrolo[1,2-b]pyridazin-4- |
| | yl)-4-methyl-2-oxopyrrolidine-3-carbonitrile |
| | 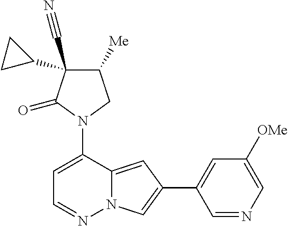 [SMILES: COc1cncc(-c2cc3c(N4C[C@@H](C)[C@@](C#N)(C5CC5)C4=O)ccnn3c2)c1] |
| | Boronate/Boronic acid: (5-methoxypyridin-3-yl)boronic acid; prep-HPLC-16; |
| | LCMS m/z = 388.2 [M + H]⁺ |
| 83 | (3R,4S)-3-cyclopropyl-4-methyl-1-(6-(1-methyl-6-oxo-1,6-dihydropyridin-3- |
| | yl)pyrrolo[1,2-b]pyridazin-4-yl)-2-oxopyrrolidine-3-carbonitrile |
| | 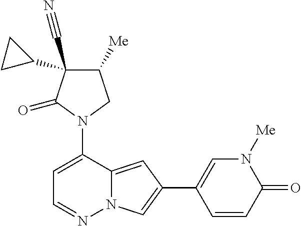 [SMILES: C[C@@H]1CN(c2ccnn3cc(-c4ccc(=O)n(C)c4)cc23)C(=O)[C@]1(C#N)C1CC1] |
| | Boronate/Boronic acid: 1-methyl-5-(4,4,5,5-tetramethyl-1,3,2-dioxaborolan-2- |
| | yl)pyridin-2(1H)-one; prep-HPLC-16; LCMS m/z = 388.2 [M+H]+ |
| 84 | (3R,4S)-3-cyclopropyl-1-(6-(5-fluoropyridin-3-yl)pyrrolo[1,2-b]pyridazin-4- |
| | yl)-4-methyl-2-oxopyrrolidine-3-carbonitrile |
| | 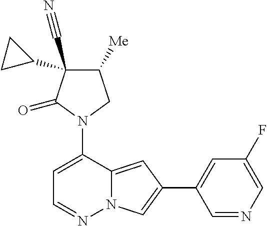 [SMILES: C[C@@H]1CN(c2ccnn3cc(-c4cncc(F)c4)cc23)C(=O)[C@]1(C#N)C1CC1] |
| | Boronate/Boronic acid: (5-fluoropyridin-3-yl)boronic acid; prep-HPLC-16; |
| | LCMS m/z = 376.2 [M + H]⁺ |
| 85 | 5-(4-((3R,4S)-3-cyano-3-cyclopropyl-4-methyl-2-oxopyrrolidin-1- |
| | yl)pyrrolo[1,2-b]pyridazin-6-yl)-2-methoxynicotinonitrile |
| | 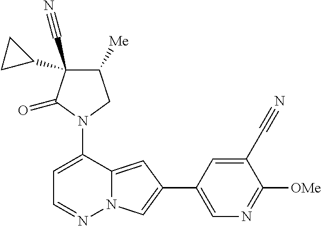 [SMILES: COc1ncc(-c2cc3c(N4C[C@@H](C)[C@@](C#N)(C5CC5)C4=O)ccnn3c2)cc1C#N] |
| | Boronate/Boronic acid: 2-methoxy-5-(4,4,5,5-tetramethyl-1,3,2-dioxaborolan- |
| | 2-yl)nicotinonitrile; Precipitate; LCMS m/z = 413.2 [M + H]⁺ |
| 86 | 5-(4-((3R,4S)-3-cyano-3-cyclopropyl-4-methyl-2-oxopyrrolidin-1- |
| | yl)pyrrolo[1,2-b]pyridazin-6-yl)picolinonitrile trifluoroacetate |
| | 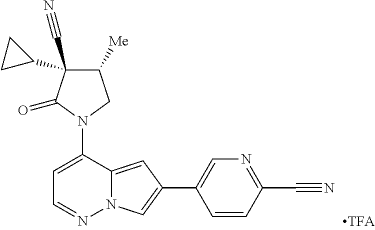 [SMILES: C[C@@H]1CN(c2ccnn3cc(-c4ccc(C#N)nc4)cc23)C(=O)[C@]1(C#N)C1CC1] |
| | Boronate/Boronic acid: (6-cyanopyridin-3-yl)boronic acid; prep-HPLC-15; |
| | LCMS m/z = 384.2 [M + H]⁺ |
| 87 | (3R,4S)-1-(6-(5-chloropyridin-3-yl)pyrrolo[1,2-b]pyridazin-4-yl)-3- |
| | cyclopropyl-4-methyl-2-oxopyrrolidine-3-carbonitrile |
| | 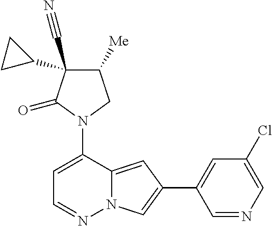 [SMILES: C[C@@H]1CN(c2ccnn3cc(-c4cncc(Cl)c4)cc23)C(=O)[C@]1(C#N)C1CC1] |
| | Boronate/Boronic acid: (5-chloropyridin-3-yl)boronic acid; prep-HPLC-16; |
| | LCMS m/z = 392.2 [M + H]⁺ |
| 88 | (3R,4S)-3-cyclopropyl-1-(6-(imidazo[1,2-a]pyridin-6-yl)pyrrolo[1,2- |
| | b]pyridazin-4-yl)-4-methyl-2-oxopyrrolidine-3-carbonitrile |
| | 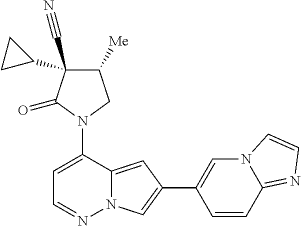 [SMILES: C[C@@H]1CN(c2ccnn3cc(-c4ccc5nccn5c4)cc23)C(=O)[C@]1(C#N)C1CC1] |
| | Boronate/Boronic acid: imidazo[1,2-a]pyridin-6-ylboronic acid; prep-HPLC- |
| | 16; LCMS m/z = 397.2 [M + H]⁺ |
| 89 | (3R,4S)-3-cyclopropyl-4-methyl-1-(6-(1-methyl-1H-imidazol-5-yl)pyrrolo[1,2- |
| | b]pyridazin-4-yl)-2-oxopyrrolidine-3-carbonitrile |
| | 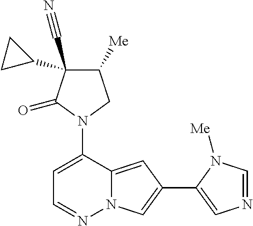 [SMILES: C[C@@H]1CN(c2ccnn3cc(-c4cncn4C)cc23)C(=O)[C@]1(C#N)C1CC1] |
| | Boronate/Boronic acid: 1-methyl-5-(4,4,5,5-tetramethyl-1,3,2-dioxaborolan-2- |
| | yl)-1H-imidazole; prep-HPLC-16; LCMS m/z = 361.2 [M + H]⁺ |
| 90 | N-(5-(4-((3R,4S)-3-cyano-3-cyclopropyl-4-methyl-2-oxopyrrolidin-1- |
| | yl)pyrrolo[1,2-b]pyridazin-6-yl)pyridin-3-yl)acetamide |
| | 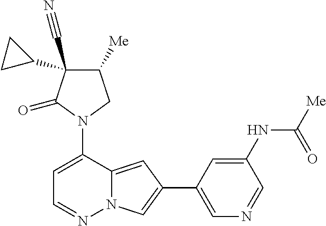 [SMILES: CC(=O)Nc1cncc(-c2cc3c(N4C[C@@H](C)[C@@](C#N)(C5CC5)C4=O)ccnn3c2)c1] |
| | Boronate/Boronic acid: N-(5-(4,4,5,5-tetramethyl-1,3,2-dioxaborolan-2- |
| | yl)pyridin-3-yl)acetamide; prep-HPLC-16; LCMS m/z = 415.2 [M + H]⁺ |
| 91 | (3R,4S)-3-cyclopropyl-1-(6-(2-(dimethylamino)pyrimidin-5-yl)pyrrolo[1,2- |
| | b]pyridazin-4-yl)-4-methyl-2-oxopyrrolidine-3-carbonitrile |
| | 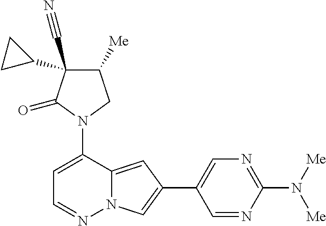 [SMILES: C[C@@H]1CN(c2ccnn3cc(-c4cnc(N(C)C)nc4)cc23)C(=O)[C@]1(C#N)C1CC1] |
| | Boronate/Boronic acid: (2-(dimethylamino)pyrimidin-5-yl)boronic acid; prep- |
| | HPLC-16; LCMS m/z = 402.2 [M + H]⁺ |
| 92 | (3R,4S)-3-cyclopropyl-4-methyl-2-oxo-1-(6-(pyrimidin-5-yl)pyrrolo[1,2- |
| | b]pyridazin-4-yl)pyrrolidine-3-carbonitrile |
| | 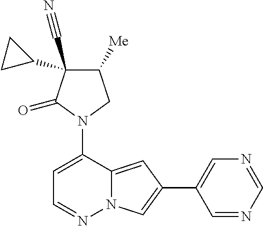 [SMILES: C[C@@H]1CN(c2ccnn3cc(-c4cncnc4)cc23)C(=O)[C@]1(C#N)C1CC1] |
| | Boronate/Boronic acid: pyrimidin-5-ylboronic acid; prep-HPLC-16; LCMS |
| | m/z = 359.2 [M + H]⁺ |
| 93 | 4-(4-((3R,4S)-3-cyano-3-cyclopropyl-4-methyl-2-oxopyrrolidin-1- |
| | yl)pyrrolo[1,2-b]pyridazin-6-yl)picolinonitrile |
| | 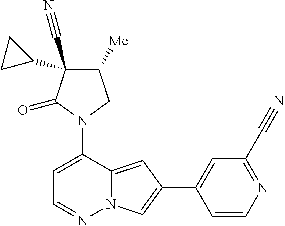 [SMILES: C[C@@H]1CN(c2ccnn3cc(-c4ccnc(C#N)c4)cc23)C(=O)[C@]1(C#N)C1CC1] |
| | Boronate/Boronic acid: 4-(4,4,5,5-tetramethyl-1,3,2-dioxaborolan-2- |
| | yl)picolinonitrile; prep-HPLC-16; LCMS m/z = 383.1 [M + H]⁺ |
| 94 | (3R,4S)-3-cyclopropyl-1-(6-(5-fluoro-2-methoxypyridin-4-yl)pyrrolo[1,2- |
| | b]pyridazin-4-yl)-4-methyl-2-oxopyrrolidine-3-carbonitrile |
| | 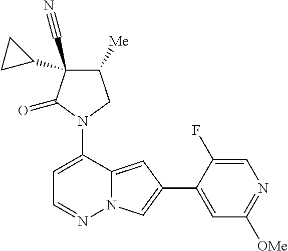 [SMILES: COc1cc(-c2cc3c(N4C[C@@H](C)[C@@](C#N)(C5CC5)C4=O)ccnn3c2)c(F)cn1] |
| | Boronate/Boronic acid: (5-fluoro-2-methoxypyridin-4-yl)boronic acid; prep- |
| | HPLC-16; LCMS m/z = 406.1 [M + H]⁺ |
| 95 | tert-butyl (5-(4-((3R,4S)-3-cyano-3-cyclopropyl-4-methyl-2-oxopyrrolidin-1- |
| | yl)pyrrolo[1,2-b]pyridazin-6-yl)pyridin-2-yl)(methyl)carbamate |
| | 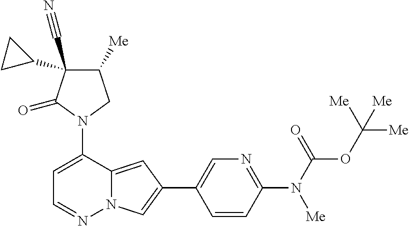 [SMILES: C[C@@H]1CN(c2ccnn3cc(-c4ccc(N(C)C(=O)OC(C)(C)C)nc4)cc23)C(=O)[C@]1(C#N)C1CC1] |
| | Boronate/Boronic acid: tert-butyl methyl(5-(4,4,5,5-tetramethyl-1,3,2- |
| | dioxaborolan-2-yl)pyridin-2-yl)carbamate; prep-HPLC-16; LCMS m/z = 487.3 |
| | [M + H]⁺ |
| 96 | (3R,4S)-3-cyclopropyl-1-(6-(2-methoxypyridin-3-yl)pyrrolo[1,2-b]pyridazin-4- |
| | yl)-4-methyl-2-oxopyrrolidine-3-carbonitrile |
| | 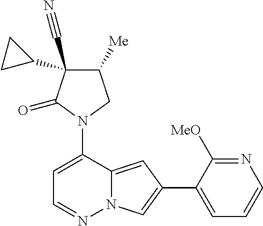 [SMILES: COc1ncccc1-c1cc2c(N3C[C@@H](C)[C@@](C#N)(C4CC4)C3=O)ccnn2c1] |
| | Boronate/Boronic acid: (2-methoxypyridin-3-yl)boronic acid; prep-HPLC-16; |
| | LCMS m/z = 388.1 [M + H]⁺ |
| 97 | (3R,4S)-3-cyclopropyl-4-methyl-1-(6-(1-(oxetan-3-yl)-1H-pyrazol-4- |
| | yl)pyrrolo[1,2-b]pyridazin-4-yl)-2-oxopyrrolidine-3-carbonitrile |
| | 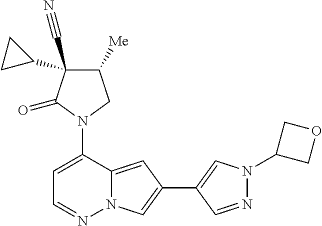 [SMILES: C[C@@H]1CN(c2ccnn3cc(-c4cnn(C5COC5)c4)cc23)C(=O)[C@]1(C#N)C1CC1] |
| | Boronate/Boronic acid: 1-(oxetan-3-yl)-4-(4,4,5,5-tetramethyl-1,3,2- |
| | dioxaborolan-2-yl)-1H-pyrazole; prep-HPLC-16; LCMS m/z = 403.2 [M + H]⁺ |
| 98 | (3R,4S)-3-cyclopropyl-1-(6-(3,6-dimethoxypyridazin-4-yl)pyrrolo[1,2- |
| | b]pyridazin-4-yl)-4-methyl-2-oxopyrrolidine-3-carbonitrile |
| | 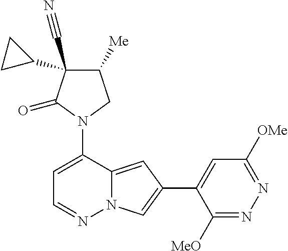 [SMILES: COc1cc(-c2cc3c(N4C[C@@H](C)[C@@](C#N)(C5CC5)C4=O)ccnn3c2)c(OC)nn1] |
| | Boronate/Boronic acid: (3,6-dimethoxypyridazin-4-yl)boronic acid; Precipitate; |
| | LCMS m/z = 419.2 [M + H]⁺ |

### Example 99-152

The title compounds were prepared from (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69) and the appropriate halide (R-X) using a one-step library protocol described below. Specific method (Method 1, 2, 3 or 4) noted in the table.

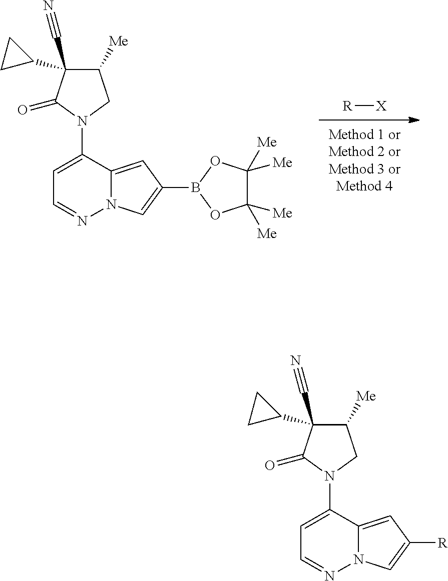

Method 1: To a mixture of (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 60.9 mg, 0.15 mmol, 1 eq) in dioxane (3.0 mL) was added the appropriate halide (0.225 mmol, 1.5 eq), K₃PO₄ (150 □l, 0.3 mmol, 2M, 2 eq) and Xphos Pd G3 (6.35 mg, 0.008 mmol, 0.05 eq) under N₂ atmosphere. The reaction mixture was shaken at 100° C. for 16 hrs. The reaction mixture was concentrated under reduced pressure and the residue purified by prep-HPLC-15 (gradient optimised for each example) to give the title compound.

Method 2: As for Method 1 except shaken at 60° C. for 5 hrs.

Method 3: As for Method 1 except Pd-132 catalyst at 60° C. for 5 hrs.

Method 4: As for Method 1 except Pd(dppf)Cl₂·DCM catalyst at 100° C. for 16 hrs.

| Example No. | Name/Structure/Method/HPLC/Reactants/Data |
|---|---|
| 99 | (3R,4S)-3-cyclopropyl-4-methyl-1-(6-(2-methyl-2H-1,2,3-triazol-4- |
| | yl)pyrrolo[1,2-b]pyridazin-4-yl)-2-oxopyrrolidine-3-carbonitrile |
| | trifluoroacetate |
| | 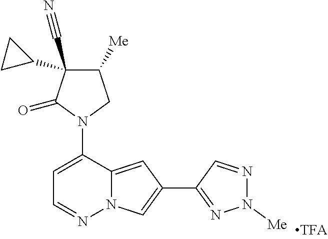 [SMILES: C[C@@H]1CN(c2ccnn3cc(-c4cnn(C)n4)cc23)C(=O)[C@]1(C#N)C1CC1] |
| | Halide: 4-bromo-2-methyl-2H-1,2,3-triazole; Method: 1; LCMS m/z = 362.2 |
| | [M + H]⁺ |
| 100 | (3R,4S)-3-cyclopropyl-4-methyl-2-oxo-1-(6-(5,6,7,8-tetrahydroimidazo[1,2- |
| | a]pyridin-3-yl)pyrrolo[1,2-b]pyridazin-4-yl)pyrrolidine-3-carbonitrile |
| | trifluoroacetate |
| | 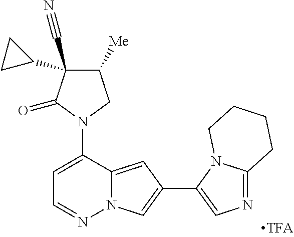 [SMILES: C[C@@H]1CN(c2ccnn3cc(-c4cnc5n4CCCC5)cc23)C(=O)[C@]1(C#N)C1CC1] |
| | Halide: 3-bromo-5,6,7,8-tetrahydroimidazo[1,2-a]pyridine |
| | Method: 1 |
| | LCMS m/z = 401.2 [M + H]⁺ |
| 101 | (3R,4S)-3-cyclopropyl-1-(6-(2-(difluoromethyl)thiazol-5-yl)pyrrolo[1,2- |
| | b]pyridazin-4-yl)-4-methyl-2-oxopyrrolidine-3-carbonitrile trifluoroacetate |
| | 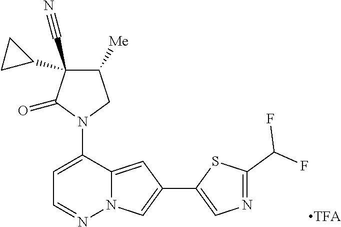 [SMILES: C[C@@H]1CN(c2ccnn3cc(-c4cnc(C(F)F)s4)cc23)C(=O)[C@]1(C#N)C1CC1] |
| | Halide: 5-bromo-2-(difluoromethyl)thiazole |
| | Method: 1 |
| | LCMS m/z = 414.1 [M + H]⁺ |
| 102 | (3R,4S)-3-cyclopropyl-4-methyl-2-oxo-1-(6-(thiazolo[4,5-c]pyridin-2- |
| | yl)pyrrolo[1,2-b]pyridazin-4-yl)pyrrolidine-3-carbonitrile trifluoroacetate |
| | 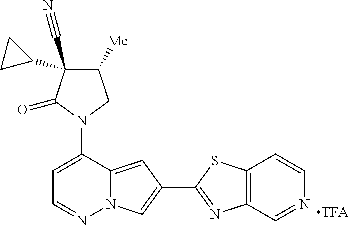 [SMILES: C[C@@H]1CN(c2ccnn3cc(-c4nc5cnccc5s4)cc23)C(=O)[C@]1(C#N)C1CC1] |
| | Halide: 2-chlorothiazolo[4,5-c]pyridine |
| | Method: 1 |
| | LCMS m/z = 415.1 [M + H]⁺ |
| 103 | 3-(4-((3R,4S)-3-cyano-3-cyclopropyl-4-methyl-2-oxopyrrolidin-1- |
| | yl)pyrrolo[1,2-b]pyridazin-6-yl)imidazo[1,2-a]pyridine-7-carbonitrile |
| | trifluoroacetate |
| | 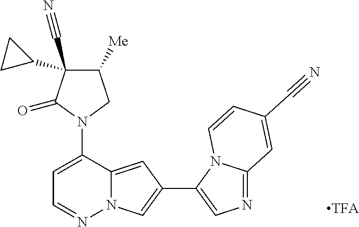 [SMILES: C[C@@H]1CN(c2ccnn3cc(-c4cnc5cc(C#N)ccn45)cc23)C(=O)[C@]1(C#N)C1CC1] |
| | Halide: 3-bromoimidazo[1,2-a]pyridine-7-carbonitrile |
| | Method: 1 |
| | LCMS m/z = 422.2 [M + H]⁺ |
| 104 | (3R,4S)-3-cyclopropyl-4-methyl-2-oxo-1-(6-(pyrazolo[1,5-a]pyridin-2- |
| | yl)pyrrolo[1,2-b]pyridazin-4-yl)pyrrolidine-3-carbonitrile trifluoroacetate |
| | 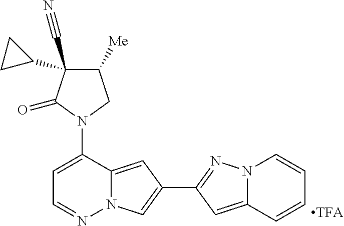 [SMILES: C[C@@H]1CN(c2ccnn3cc(-c4cc5ccccn5n4)cc23)C(=O)[C@]1(C#N)C1CC1] |
| | Halide: 2-chloropyrazolo[1,5-a]pyridine |
| | Method: 3 |
| | LCMS m/z = 397.2 [M + H]⁺ |
| 105 | (3R,4S)-3-cyclopropyl-4-methyl-2-oxo-1-(6-(5,6,7,8-tetrahydro- |
| | [1,2,4]triazolo[4,3-a]pyridin-3-yl)pyrrolo[1,2-b]pyridazin-4-yl)pyrrolidine-3- |
| | carbonitrile trifluoroacetate |
| | 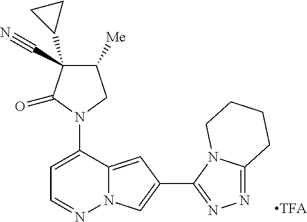 [SMILES: C[C@@H]1CN(c2ccnn3cc(-c4nnc5n4CCCC5)cc23)C(=O)[C@]1(C#N)C1CC1] |
| | Halide: 3-bromo-5,6,7,8-tetrahydro-[1,2,4]triazolo[4,3-a]pyridine |
| | Method: 1 |
| | LCMS m/z = 402.2 [M + H]⁺ |
| 106 | (3R,4S)-3-cyclopropyl-1-(6-(6,7-dihydro-5H-pyrrolo[1,2-a]imidazol-2- |
| | yl)pyrrolo[1,2-b]pyridazin-4-yl)-4-methyl-2-oxopyrrolidine-3-carbonitrile |
| | trifluoroacetate |
| | 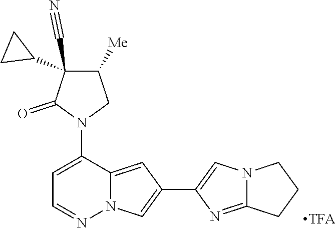 [SMILES: C[C@@H]1CN(c2ccnn3cc(-c4cn5c(n4)CCC5)cc23)C(=O)[C@]1(C#N)C1CC1] |
| | Halide: 2-bromo-6,7-dihydro-5H-pyrrolo[1,2-a]imidazole |
| | Method: 1 |
| | LCMS m/z = 387.2 [M + H]⁺ |
| 107 | (3R,4S)-3-cyclopropyl-4-methyl-1-(6-(1-methyl-3-(trifluoromethyl)-1H- |
| | pyrazol-5-yl)pyrrolo[1,2-b]pyridazin-4-yl)-2-oxopyrrolidine-3-carbonitrile |
| | trifluoroacetate |
| | 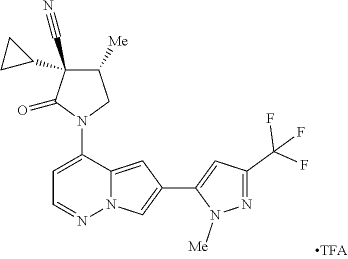 [SMILES: C[C@@H]1CN(c2ccnn3cc(-c4cc(C(F)(F)F)nn4C)cc23)C(=O)[C@]1(C#N)C1CC1] |
| | Halide: 5-bromo-1-methyl-3-(trifluoromethyl)-1H-pyrazole |
| | Method: 1 |
| | LCMS m/z = 429.1 [M + H]⁺ |
| 108 | (3R,4S)-1-(6-([1,2,4]triazolo[1,5-a]pyridin-2-yl)pyrrolo[1,2-b]pyridazin-4-yl)- |
| | 3-cyclopropyl-4-methyl-2-oxopyrrolidine-3-carbonitrile trifluoroacetate |
| | 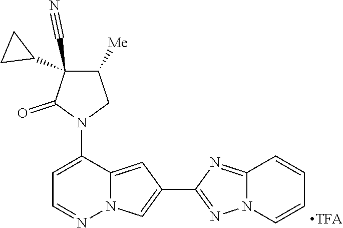 [SMILES: C[C@@H]1CN(c2ccnn3cc(-c4nc5ccccn5n4)cc23)C(=O)[C@]1(C#N)C1CC1] |
| | Halide: 2-chloro-[1,2,4]triazolo[1,5-a]pyridine |
| | Method: 3 |
| | LCMS m/z = 398.3 [M + H]⁺ |
| 109 | (3R,4S)-3-cyclopropyl-4-methyl-1-(6-(6-methyl-[1,2,4]triazolo[1,5-a]pyridin- |
| | 2-yl)pyrrolo[1,2-b]pyridazin-4-yl)-2-oxopyrrolidine-3-carbonitrile |
| | trifluoroacetate |
| | 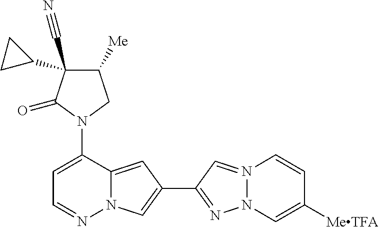 |
| | Halide: 2-chloro-6-methyl-[1,2,4]triazolo[1,5-a]pyridine |
| | Method: 1 |
| | LCMS m/z = 412.2 [M + H]⁺ |
| 110 | (3R,4S)-3-cyclopropyl-1-(6-(2,3-dihydropyrazolo[5,1-b]oxazol-6- |
| | yl)pyrrolo[1,2-b]pyridazin-4-yl)-4-methyl-2-oxopyrrolidine-3-carbonitrile |
| | trifluoroacetate |
| | 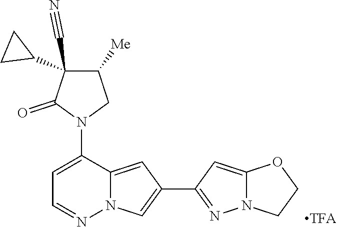 [SMILES: C[C@@H]1CN(c2ccnn3cc(-c4cc5n(n4)CCO5)cc23)C(=O)[C@]1(C#N)C1CC1] |
| | Halide: 6-bromo-2,3-dihydropyrazolo[5,1-b]oxazole |
| | Method: 3 |
| | LCMS m/z = 389.3 [M + H]⁺ |
| 111 | (3R,4S)-3-cyclopropyl-4-methyl-1-(6-(1-methyl-1H-imidazol-4-yl)pyrrolo[1,2- |
| | b]pyridazin-4-yl)-2-oxopyrrolidine-3-carbonitrile trifluoroacetate |
| | 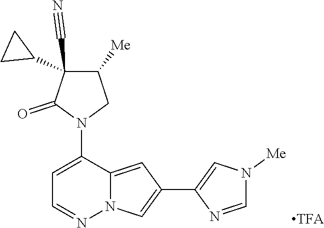 [SMILES: C[C@@H]1CN(c2ccnn3cc(-c4cn(C)cn4)cc23)C(=O)[C@]1(C#N)C1CC1] |
| | Halide: 4-bromo-1-methyl-1H-imidazole |
| | Method: 1 |
| | LCMS m/z = 361.2 [M + H]⁺ |
| 112 | (3R,4S)-3-cyclopropyl-4-methyl-1-(6-(1-methyl-1H-1,2,3-triazol-4- |
| | yl)pyrrolo[1,2-b]pyridazin-4-yl)-2-oxopyrrolidine-3-carbonitrile |
| | trifluoroacetate |
| | 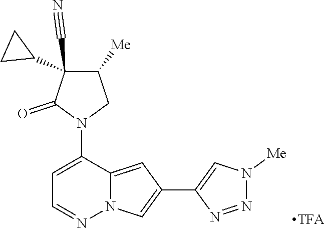 [SMILES: C[C@@H]1CN(c2ccnn3cc(-c4cn(C)nn4)cc23)C(=O)[C@]1(C#N)C1CC1] |
| | Halide: 4-bromo-1-methyl-1H-1,2,3-triazole |
| | Method: 3 |
| | LCMS m/z = 362.3 [M + H]⁺ |
| 113 | (3R,4S)-3-cyclopropyl-4-methyl-1-(6-(4-methylthiazol-2-yl)pyrrolo[1,2- |
| | b]pyridazin-4-yl)-2-oxopyrrolidine-3-carbonitrile trifluoroacetate |
| | 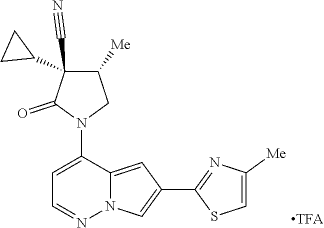 [SMILES: Cc1csc(-c2cc3c(N4C[C@@H](C)[C@@](C#N)(C5CC5)C4=O)ccnn3c2)n1] |
| | Halide: 2-chloro-4-methylthiazole |
| | Method: 3 |
| | LCMS m/z = 378.2 [M + H]⁺ |
| 114 | 2-(4-((3R,4S)-3-cyano-3-cyclopropyl-4-methyl-2-oxopyrrolidin-1- |
| | yl)pyrrolo[1,2-b]pyridazin-6-yl)thiazole-4-carbonitrile trifluoroacetate |
| | 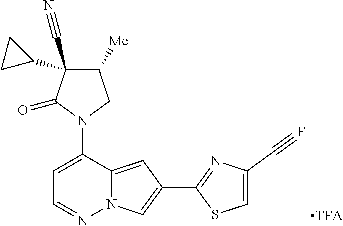 |
| | Halide: 2-bromothiazole-4-carbonitrile |
| | Method: 1 |
| | LCMS m/z = 389.1 [M + H]⁺ |
| 115 | (3R,4S)-3-cyclopropyl-4-methyl-1-(6-(1-methyl-1H-1,2,4-triazol-3- |
| | yl)pyrrolo[1,2-b]pyridazin-4-yl)-2-oxopyrrolidine-3-carbonitrile |
| | trifluoroacetate |
| | 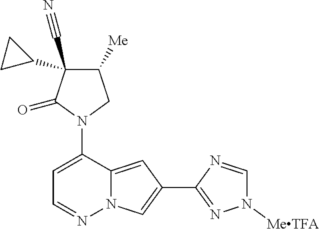 |
| | Halide: 3-bromo-1-methyl-1H-1,2,4-triazole |
| | Method: 3 |
| | LCMS m/z = 362.3 [M + H]⁺ |
| 116 | (3R,4S)-3-cyclopropyl-1-(6-(imidazo[1,2-a]pyrazin-3-yl)pyrrolo[1,2- |
| | b]pyridazin-4-yl)-4-methyl-2-oxopyrrolidine-3-carbonitrile trifluoroacetate |
| | 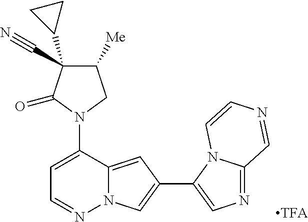 [SMILES: C[C@@H]1CN(c2ccnn3cc(-c4cnc5cnccn45)cc23)C(=O)[C@]1(C#N)C1CC1] |
| | Halide: 3-bromoimidazo[1,2-a]pyrazine |
| | Method: 3 |
| | LCMS m/z = 398.3 [M + H]⁺ |
| 117 | 2-(4-((3R,4S)-3-cyano-3-cyclopropyl-4-methyl-2-oxopyrrolidin-1- |
| | yl)pyrrolo[1,2-b]pyridazin-6-yl)thiazole-5-carbonitrile trifluoroacetate |
| | 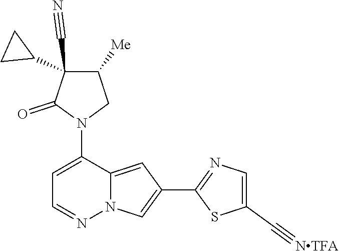 |
| | Halide: 2-chlorothiazole-5-carbonitrile |
| | Method: 1 |
| | LCMS m/z = 389.1 [M + H]⁺ |
| 118 | (3R,4S)-3-cyclopropyl-4-methyl-1-(6-(5-methylthiazol-2-yl)pyrrolo[1,2- |
| | b]pyridazin-4-yl)-2-oxopyrrolidine-3-carbonitrile trifluoroacetate |
| | 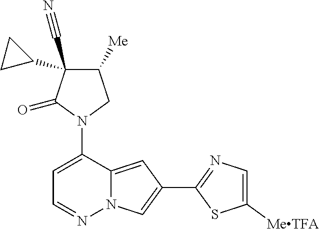 |
| | Halide: 2-bromo-5-methylthiazole |
| | Method: 3 |
| | LCMS m/z = 378.2 [M + H]⁺ |
| 119 | 6-(4-((3R,4S)-3-cyano-3-cyclopropyl-4-methyl-2-oxopyrrolidin-1- |
| | yl)pyrrolo[1,2-b]pyridazin-6-yl)-2-methylnicotinonitrile trifluoroacetate |
| | 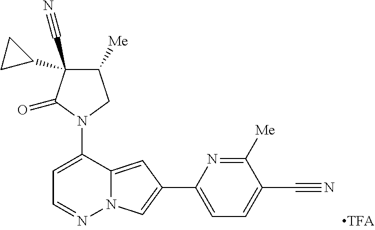 [SMILES: Cc1nc(-c2cc3c(N4C[C@@H](C)[C@@](C#N)(C5CC5)C4=O)ccnn3c2)ccc1C#N] |
| | Halide: 6-bromo-2-methylnicotinonitrile |
| | Method: 1 |
| | LCMS m/z = 397.2 [M + H]⁺ |
| 120 | (3R,4S)-3-cyclopropyl-4-methyl-2-oxo-1-(6-(4-(trifluoromethyl)pyrimidin-2- |
| | yl)pyrrolo[1,2-b]pyridazin-4-yl)pyrrolidine-3-carbonitrile trifluoroacetate |
| | 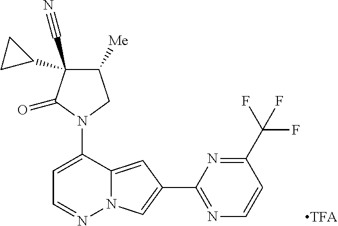 [SMILES: C[C@@H]1CN(c2ccnn3cc(-c4nccc(C(F)(F)F)n4)cc23)C(=O)[C@]1(C#N)C1CC1] |
| | Halide: 2-bromo-4-(trifluoromethyl)pyrimidine |
| | Method: 1 |
| | LCMS m/z = 427.1 [M + H]⁺ |
| 121 | (3R,4S)-3-cyclopropyl-4-methyl-2-oxo-1-(6-(6-(trifluoromethyl)pyrimidin-4- |
| | yl)pyrrolo[1,2-b]pyridazin-4-yl)pyrrolidine-3-carbonitrile trifluoroacetate |
| | 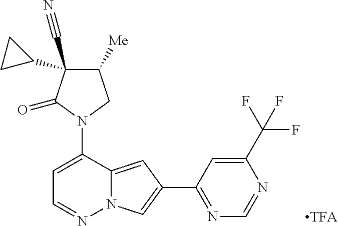 [SMILES: C[C@@H]1CN(c2ccnn3cc(-c4cc(C(F)(F)F)ncn4)cc23)C(=O)[C@]1(C#N)C1CC1] |
| | Halide: 4-bromo-6-(trifluoromethyl)pyrimidine |
| | Method: 2 |
| | LCMS m/z = 427.2 [M + H]⁺ |
| 122 | (3R,4S)-3-cyclopropyl-1-(6-(4-methoxypyrimidin-2-yl)pyrrolo[1,2- |
| | b]pyridazin-4-yl)-4-methyl-2-oxopyrrolidine-3-carbonitrile trifluoroacetate |
| | 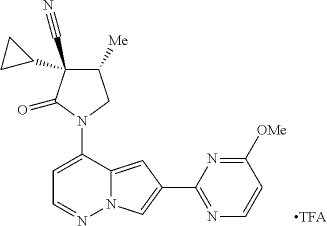 [SMILES: COc1ccnc(-c2cc3c(N4C[C@@H](C)[C@@](C#N)(C5CC5)C4=O)ccnn3c2)n1] |
| | Halide: 2-bromo-4-methoxypyrimidine |
| | Method: 1 |
| | LCMS m/z = 389.2 [M + H]⁺ |
| 123 | (3R,4S)-3-cyclopropyl-1-(6-(imidazo[1,2-a]pyrazin-8-yl)pyrrolo[1,2- |
| | b]pyridazin-4-yl)-4-methyl-2-oxopyrrolidine-3-carbonitrile trifluoroacetate |
| | 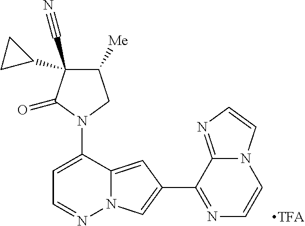 [SMILES: C[C@@H]1CN(c2ccnn3cc(-c4nccn5ccnc45)cc23)C(=O)[C@]1(C#N)C1CC1] |
| | Halide: 8-bromoimidazo[1,2-a]pyrazine |
| | Method: 2 |
| | LCMS m/z = 398.3 [M + H]⁺ |
| 124 | 6-(4-((3R,4S)-3-cyano-3-cyclopropyl-4-methyl-2-oxopyrrolidin-1- |
| | yl)pyrrolo[1,2-b]pyridazin-6-yl)-N,N-dimethylnicotinamide trifluoroacetate |
| | 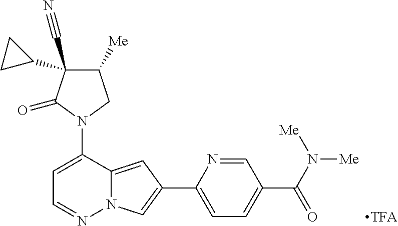 [SMILES: C[C@@H]1CN(c2ccnn3cc(-c4ccc(C(=O)N(C)C)cn4)cc23)C(=O)[C@]1(C#N)C1CC1] |
| | Halide: 6-bromo-N,N-dimethylnicotinamide |
| | Method: 2 |
| | LCMS m/z = 429.3 [M + H]⁺ |
| 125 | (3R,4S)-1-(6-(6-(cyanomethyl)pyridin-2-yl)pyrrolo[1,2-b]pyridazin-4-yl)-3- |
| | cyclopropyl-4-methyl-2-oxopyrrolidine-3-carbonitrile trifluoroacetate |
| | 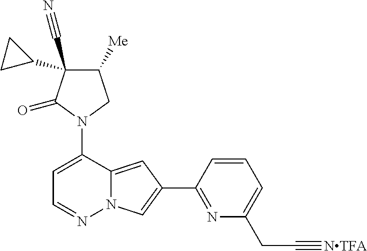 |
| | Halide: 2-(6-bromopyridin-2-yl)acetonitrile |
| | Method: 1 |
| | LCMS m/z = 397.2 [M + H]⁺ |
| 126 | (3R,4S)-3-cyclopropyl-1-(6-(6-methoxypyrazin-2-yl)pyrrolo[1,2-b]pyridazin- |
| | 4-yl)-4-methyl-2-oxopyrrolidine-3-carbonitrile trifluoroacetate |
| | 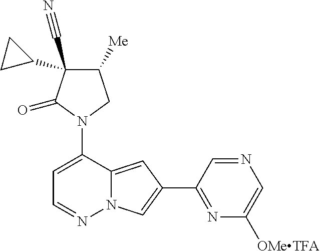 |
| | Halide: 2-bromo-6-methoxypyrazine |
| | Method: 1 |
| | LCMS m/z = 389.2 [M + H]⁺ |
| 127 | (3R,4S)-1-(6-(5-(cyanomethyl)pyridin-2-yl)pyrrolo[1,2-b]pyridazin-4-yl)-3- |
| | cyclopropyl-4-methyl-2-oxopyrrolidine-3-carbonitrile trifluoroacetate |
| | 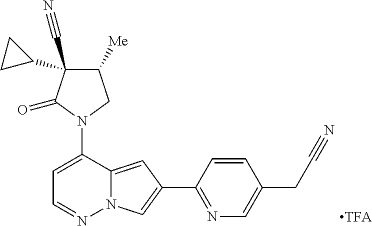 [SMILES: C[C@@H]1CN(c2ccnn3cc(-c4ccc(CC#N)cn4)cc23)C(=O)[C@]1(C#N)C1CC1] |
| | Halide: 2-(6-bromopyridin-3-yl)acetonitrile |
| | Method: 1 |
| | LCMS m/z = 397.2 [M + H]⁺ |
| 128 | (3R,4S)-3-cyclopropyl-1-(6-(imidazo[1,5-a]pyridin-5-yl)pyrrolo[1,2- |
| | b]pyridazin-4-yl)-4-methyl-2-oxopyrrolidine-3-carbonitrile trifluoroacetate |
| | 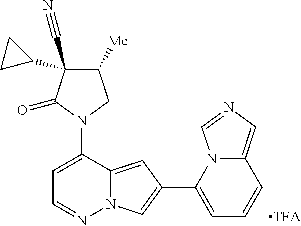 [SMILES: C[C@@H]1CN(c2ccnn3cc(-c4cccc5cncn45)cc23)C(=O)[C@]1(C#N)C1CC1] |
| | Halide: 5-bromoimidazo[1,5-a]pyridine |
| | Method: 1 |
| | LCMS m/z = 397.2 [M + H]⁺ |
| 129 | 2-(4-((3R,4S)-3-cyano-3-cyclopropyl-4-methyl-2-oxopyrrolidin-1- |
| | yl)pyrrolo[1,2-b]pyridazin-6-yl)-4-hydroxynicotinonitrile trifluoroacetate |
| | 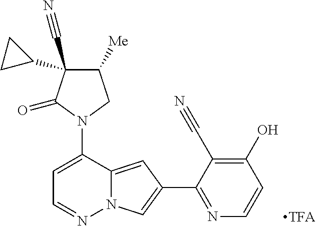 [SMILES: C[C@@H]1CN(c2ccnn3cc(-c4nccc(O)c4C#N)cc23)C(=O)[C@]1(C#N)C1CC1] |
| | Halide: 2-bromo-4-hydroxynicotinonitrile |
| | Method: 1 |
| | LCMS m/z = 399.1 [M + H]⁺ |
| 130 | (3R,4S)-3-cyclopropyl-1-(6-(5-(1-hydroxyethyl)pyridin-2-yl)pyrrolo[1,2- |
| | b]pyridazin-4-yl)-4-methyl-2-oxopyrrolidine-3-carbonitrile trifluoroacetate |
| | 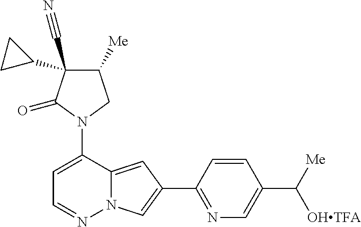 |
| | Halide: 1-(6-bromopyridin-3-yl)ethan-1-ol |
| | Method: 1 |
| | LCMS m/z = 402.2 [M + H]⁺ |
| 131 | N-(6-(4-((3R,4S)-3-cyano-3-cyclopropyl-4-methyl-2-oxopyrrolidin-1- |
| | yl)pyrrolo[1,2-b]pyridazin-6-yl)pyridin-2-yl)acetamide trifluoroacetate |
| | 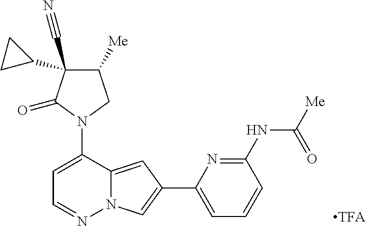 [SMILES: CC(=O)Nc1cccc(-c2cc3c(N4C[C@@H](C)[C@@](C#N)(C5CC5)C4=O)ccnn3c2)n1] |
| | Halide: N-(6-bromopyridin-2-yl)acetamide |
| | Method: 1 |
| | LCMS m/z = 415.2 [M + H]⁺ |
| 132 | (3R,4S)-3-cyclopropyl-1-(6-(5-methoxypyrazin-2-yl)pyrrolo[1,2-b]pyridazin- |
| | 4-yl)-4-methyl-2-oxopyrrolidine-3-carbonitrile trifluoroacetate |
| | 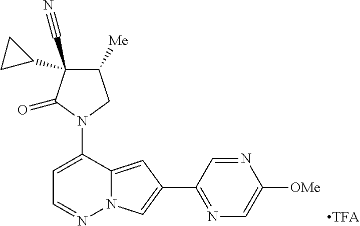 [SMILES: COc1cnc(-c2cc3c(N4C[C@@H](C)[C@@](C#N)(C5CC5)C4=O)ccnn3c2)cn1] |
| | Halide: 2-bromo-5-methoxypyrazine |
| | Method: 2 |
| | LCMS m/z = 389.3 [M + H]⁺ |
| 133 | 6-(4-((3R,4S)-3-cyano-3-cyclopropyl-4-methyl-2-oxopyrrolidin-1- |
| | yl)pyrrolo[1,2-b]pyridazin-6-yl)pyrazine-2-carbonitrile trifluoroacetate |
| | 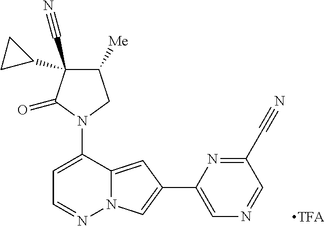 [SMILES: C[C@@H]1CN(c2ccnn3cc(-c4cncc(C#N)n4)cc23)C(=O)[C@]1(C#N)C1CC1] |
| | Halide: 6-bromopyrazine-2-carbonitrile |
| | Method: 1 |
| | LCMS m/z = 384.2 [M + H]⁺ |
| 134 | (3R,4S)-3-cyclopropyl-1-(6-(5-(dimethylamino)pyridin-2-yl)pyrrolo[1,2- |
| | b]pyridazin-4-yl)-4-methyl-2-oxopyrrolidine-3-carbonitrile trifluoroacetate |
| | 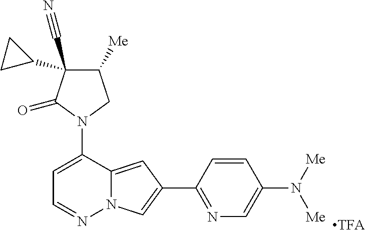 [SMILES: C[C@@H]1CN(c2ccnn3cc(-c4ccc(N(C)C)cn4)cc23)C(=O)[C@]1(C#N)C1CC1] |
| | Halide: 6-bromo-N,N-dimethylpyridin-3-amine |
| | Method: 1 |
| | LCMS m/z = 401.2 [M + H]⁺ |
| 135 | (3R,4S)-3-cyclopropyl-1-(6-(6-(difluoromethyl)pyridin-2-yl)pyrrolo[1,2- |
| | b]pyridazin-4-yl)-4-methyl-2-oxopyrrolidine-3-carbonitrile trifluoroacetate |
| | 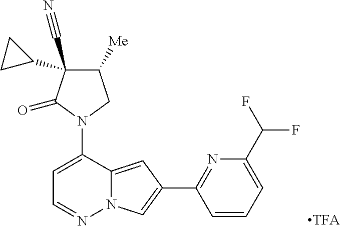 [SMILES: C[C@@H]1CN(c2ccnn3cc(-c4cccc(C(F)F)n4)cc23)C(=O)[C@]1(C#N)C1CC1] |
| | Halide: 2-bromo-6-(difluoromethyl)pyridine |
| | Method: 1 |
| | LCMS m/z = 408.2 [M + H]⁺ |
| 136 | (3R,4S)-3-cyclopropyl-1-(6-(5-methoxypyrimidin-2-yl)pyrrolo[1,2- |
| | b]pyridazin-4-yl)-4-methyl-2-oxopyrrolidine-3-carbonitrile trifluoroacetate |
| | 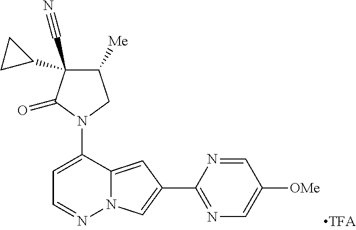 [SMILES: COc1cnc(-c2cc3c(N4C[C@@H](C)[C@@](C#N)(C5CC5)C4=O)ccnn3c2)nc1] |
| | Halide: 2-bromo-5-methoxypyrimidine |
| | Method: 2 |
| | LCMS m/z = 389.2 [M + H]⁺ |
| 137 | (3R,4S)-3-cyclopropyl-4-methyl-1-(6-(6-methylpyridazin-3-yl)pyrrolo[1,2- |
| | b]pyridazin-4-yl)-2-oxopyrrolidine-3-carbonitrile trifluoroacetate |
| | 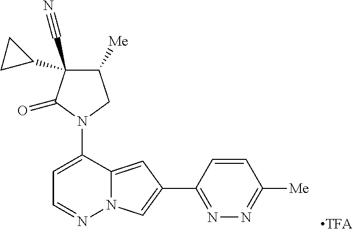 [SMILES: Cc1ccc(-c2cc3c(N4C[C@@H](C)[C@@](C#N)(C5CC5)C4=O)ccnn3c2)nn1] |
| | Halide: 3-iodo-6-methylpyridazine |
| | Method: 2 |
| | LCMS m/z = 373.3 [M + H]⁺ |
| 138 | (3R,4S)-3-cyclopropyl-1-(6-(3-fluoropyridin-2-yl)pyrrolo[1,2-b]pyridazin-4- |
| | yl)-4-methyl-2-oxopyrrolidine-3-carbonitrile trifluoroacetate |
| | 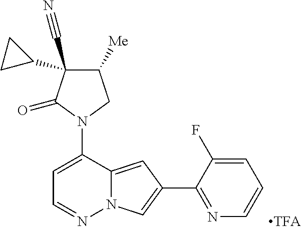 [SMILES: C[C@@H]1CN(c2ccnn3cc(-c4ncccc4F)cc23)C(=O)[C@]1(C#N)C1CC1] |
| | Halide: 3-fluoro-2-iodopyridine |
| | Method: 1 |
| | LCMS m/z = 376.2 [M + H]⁺ |
| 139 | ((3R,4S)-3-cyclopropyl-4-methyl-2-oxo-1-(6-(pyrimidin-2-yl)pyrrolo[1,2- |
| | b]pyridazin-4-yl)pyrrolidine-3-carbonitrile trifluoroacetate |
| | 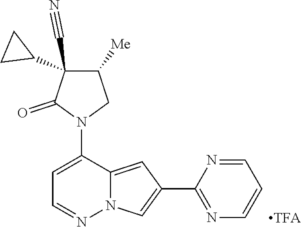 [SMILES: C[C@@H]1CN(c2ccnn3cc(-c4ncccn4)cc23)C(=O)[C@]1(C#N)C1CC1] |
| | Halide: 2-iodopyrimidine |
| | Method: 2 |
| | LCMS m/z = 359.3 [M + H]⁺ |
| 140 | (3R,4S)-3-cyclopropyl-1-(6-(3-hydroxy-6-methylpyridin-2-yl)pyrrolo[1,2- |
| | b]pyridazin-4-yl)-4-methyl-2-oxopyrrolidine-3-carbonitrile trifluoroacetate |
| | 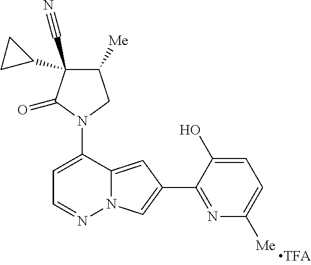 [SMILES: Cc1ccc(O)c(-c2cc3c(N4C[C@@H](C)[C@@](C#N)(C5CC5)C4=O)ccnn3c2)n1] |
| | Halide: 2-bromo-6-methylpyridin-3-ol |
| | Method: 4 |
| | LCMS m/z = 388.2 [M + H]⁺ |
| 141 | (3R,4S)-3-cyclopropyl-1-(6-(imidazo[1,2-a]pyridin-5-yl)pyrrolo[1,2- |
| | b]pyridazin-4-yl)-4-methyl-2-oxopyrrolidine-3-carbonitrile trifluoroacetate |
| | 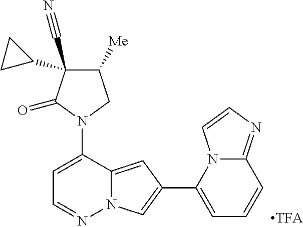 [SMILES: C[C@@H]1CN(c2ccnn3cc(-c4cccc5nccn45)cc23)C(=O)[C@]1(C#N)C1CC1] |
| | Halide: 5-bromoimidazo[1,2-a]pyridine |
| | Method: 1 |
| | LCMS m/z = 397.1 [M + H]⁺ |
| 142 | (3R,4S)-3-cyclopropyl-1-(6-(5-fluoro-4-methylpyridin-2-yl)pyrrolo[1,2- |
| | b]pyridazin-4-yl)-4-methyl-2-oxopyrrolidine-3-carbonitrile trifluoroacetate |
| | 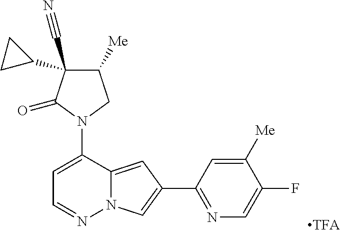 [SMILES: Cc1cc(-c2cc3c(N4C[C@@H](C)[C@@](C#N)(C5CC5)C4=O)ccnn3c2)ncc1F] |
| | Halide: 2-bromo-5-fluoro-4-methylpyridine |
| | Method: 1 |
| | LCMS m/z = 390.2 [M + H]⁺ |
| 143 | (3R,4S)-3-cyclopropyl-1-(6-(6-methoxypyridin-2-yl)pyrrolo[1,2-b]pyridazin-4- |
| | yl)-4-methyl-2-oxopyrrolidine-3-carbonitrile trifluoroacetate |
| | 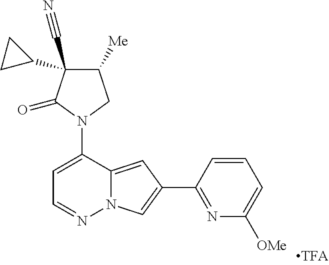 [SMILES: COc1cccc(-c2cc3c(N4C[C@@H](C)[C@@](C#N)(C5CC5)C4=O)ccnn3c2)n1] |
| | Halide: 2-iodo-6-methoxypyridine |
| | Method: 2 |
| | LCMS m/z = 388.2 [M + H]⁺ |
| 144 | 6-(4-((3R,4S)-3-cyano-3-cyclopropyl-4-methyl-2-oxopyrrolidin-1- |
| | yl)pyrrolo[1,2-b]pyridazin-6-yl)-4-methylnicotinonitrile trifluoroacetate |
| | 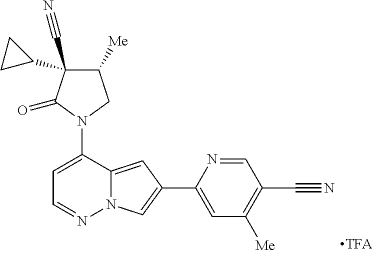 [SMILES: Cc1cc(-c2cc3c(N4C[C@@H](C)[C@@](C#N)(C5CC5)C4=O)ccnn3c2)ncc1C#N] |
| | Halide: 6-bromo-4-methylnicotinonitrile |
| | Method: 1 |
| | LCMS m/z = 397.2 [M + H]⁺ |
| 145 | (3R,4S)-3-cyclopropyl-1-(6-(5-methoxypyridin-2-yl)pyrrolo[1,2-b]pyridazin-4- |
| | yl)-4-methyl-2-oxopyrrolidine-3-carbonitrile trifluoroacetate |
| | 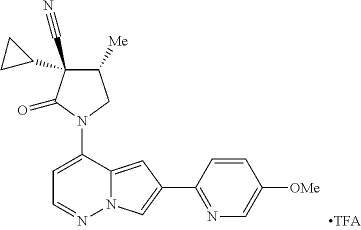 [SMILES: COc1ccc(-c2cc3c(N4C[C@@H](C)[C@@](C#N)(C5CC5)C4=O)ccnn3c2)nc1] |
| | Halide: 2-bromo-5-methoxypyridine |
| | Method: 1 |
| | LCMS m/z = 388.2 [M + H]⁺ |
| 146 | (3R,4S)-3-cyclopropyl-1-(6-(3-methoxypyridin-2-yl)pyrrolo[1,2-b]pyridazin-4- |
| | yl)-4-methyl-2-oxopyrrolidine-3-carbonitrile trifluoroacetate |
| | 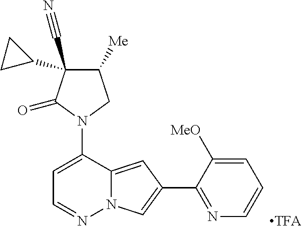 [SMILES: COc1cccnc1-c1cc2c(N3C[C@@H](C)[C@@](C#N)(C4CC4)C3=O)ccnn2c1] |
| | Halide: 2-bromo-3-methoxypyridine |
| | Method: 1 |
| | LCMS m/z = 388.2 [M + H]⁺ |
| 147 | (3R,4S)-3-cyclopropyl-1-(6-(5-fluoro-6-methylpyridin-2-yl)pyrrolo[1,2- |
| | b]pyridazin-4-yl)-4-methyl-2-oxopyrrolidine-3-carbonitrile trifluoroacetate |
| | 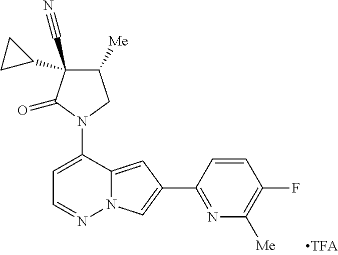 [SMILES: Cc1nc(-c2cc3c(N4C[C@@H](C)[C@@](C#N)(C5CC5)C4=O)ccnn3c2)ccc1F] |
| | Halide: 6-bromo-3-fluoro-2-methylpyridine |
| | Method: 1 |
| | LCMS m/z = 390.2 [M + H]⁺ |
| 148 | (3R,4S)-3-cyclopropyl-1-(6-(4-(hydroxymethyl)pyridin-2-yl)pyrrolo[1,2- |
| | b]pyridazin-4-yl)-4-methyl-2-oxopyrrolidine-3-carbonitrile trifluoroacetate |
| | 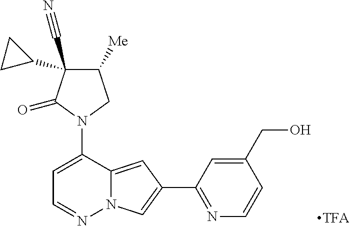 [SMILES: C[C@@H]1CN(c2ccnn3cc(-c4cc(CO)ccn4)cc23)C(=O)[C@]1(C#N)C1CC1] |
| | Halide: (2-bromopyridin-4-yl)methanol |
| | Method: 1 |
| | LCMS m/z = 388.1 [M + H]⁺ |
| 149 | (3R,4S)-3-cyclopropyl-1-(6-(3-hydroxypyridin-2-yl)pyrrolo[1,2-b]pyridazin-4- |
| | yl)-4-methyl-2-oxopyrrolidine-3-carbonitrile trifluoroacetate |
| | 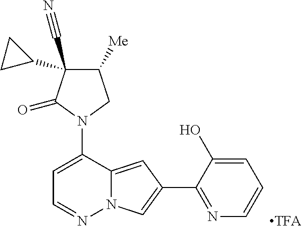 [SMILES: C[C@@H]1CN(c2ccnn3cc(-c4ncccc4O)cc23)C(=O)[C@]1(C#N)C1CC1] |
| | Halide: 2-iodopyridin-3-ol |
| | Method: 4 |
| | LCMS m/z = 374.2 [M + H]⁺ |
| 150 | (3R,4S)-3-cyclopropyl-1-(6-(5-(hydroxymethyl)pyridin-2-yl)pyrrolo[1,2- |
| | b]pyridazin-4-yl)-4-methyl-2-oxopyrrolidine-3-carbonitrile trifluoroacetate |
| | 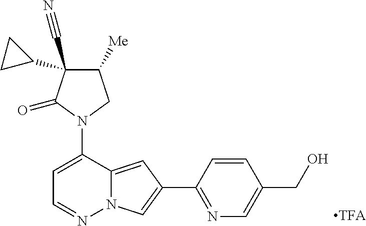 [SMILES: C[C@@H]1CN(c2ccnn3cc(-c4ccc(CO)cn4)cc23)C(=O)[C@]1(C#N)C1CC1] |
| | Halide: (6-bromopyridin-3-yl)methanol |
| | Method: 1 |
| | LCMS m/z = 388.2 [M + H]⁺ |
| 151 | (3R,4S)-3-cyclopropyl-4-methyl-2-oxo-1-(6-(pyrazin-2-yl)pyrrolo[1,2- |
| | b]pyridazin-4-yl)pyrrolidine-3-carbonitrile trifluoroacetate |
| | 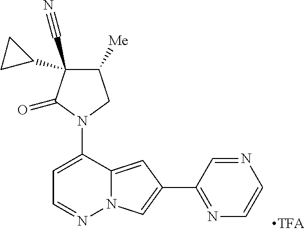 [SMILES: C[C@@H]1CN(c2ccnn3cc(-c4cnccn4)cc23)C(=O)[C@]1(C#N)C1CC1] |
| | Halide: 2-iodopyrazine |
| | Method: 1 |
| | LCMS m/z = 359.2 [M + H]⁺ |

### Example 152 and 153

### rac-(3R,4R)-3-cyclopropyl-4-(difluoromethyl)-1-(6-(1-methyl-1H-pyrazol-4-yl)pyrrolo[1,2-b]pyridazin-4-yl)-2-oxopyrrolidine-3-carbonitrile and rac-(3R,4S)-3-cyclopropyl-4-(difluoromethyl)-1-(6-(1-methyl-1H-pyrazol-4-yl)pyrrolo[1,2-b]pyridazin-4-yl)-2-oxopyrrolidine-3-carbonitrile

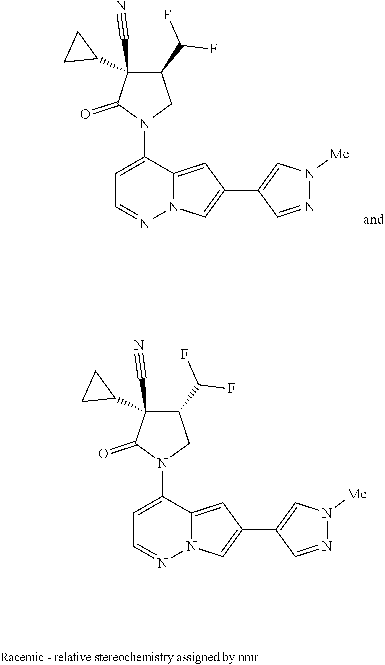 [SMILES: Cn1cc(-c2cc3c(N4C[C@H](C(F)F)[C@@](C#N)(C5CC5)C4=O)ccnn3c2)cn1 ; Cn1cc(-c2cc3c(N4C[C@@H](C(F)F)[C@@](C#N)(C5CC5)C4=O)ccnn3c2)cn1]

To a solution of 1-(6-bromopyrrolo[1,2-b]pyridazin-4-yl)-3-cyclopropyl-4-(difluoromethyl)-2-oxopyrrolidine-3-carbonitrile (Preparation 135, 60.0 mg, 0.152 mmol) in dioxane (10 mL) and H₂O (1 mL) was added 1-methyl-4-(4,4,5,5-tetramethyl-1,3,2-dioxaborolan-2-yl)-1H-pyrazole (94.8 mg, 0.455 mmol), Pd(dppf)Cl₂ (22.2 mg, 30.4 □mol) and KF (52.9 mg, 0.911 mmol) at 25° C. The mixture was stirred at 70° C. for 16 h under N₂. The mixture was evaporated to dryness in vacuo and the residue was purified by prep-HPLC-3 (Gradient: 31-61%) to give the title compounds.

Peak 1, Example 152: rac-(3R,4R)-3-cyclopropyl-4-(difluoromethyl)-1-(6-(1-methyl-1H-pyrazol-4-yl)pyrrolo[1,2-b]pyridazin-4-yl)-2-oxopyrrolidine-3-carbonitrile as a white solid (25.1 mg, 42%). LCMS m/z=397.1 [M+H]⁺; ¹H NMR (400 MHz, CDCl₃) δ: 8.00 (d, 1H), 7.92 (s, 1H), 7.73 (s, 1H), 7.62 (s, 1H), 6.74 (d, 1H), 6.55 (s, 1H), 6.29 (t, 1H), 4.22-4.26 (m, 2H), 3.96 (s, 3H), 3.06-3.11 (m, 1H), 1.37-1.39 (m, 1H), 0.80-0.91 (m, 4H).

Peak 2, Example 153: rac-(3R,4S)-3-cyclopropyl-4-(difluoromethyl)-1-(6-(1-methyl-1H-pyrazol-4-yl)pyrrolo[1,2-b]pyridazin-4-yl)-2-oxopyrrolidine-3-carbonitrile as a green solid (8.7 mg, 14%). LCMS m/z=397.1 [M+H]⁺; ¹H NMR (400 MHz, CDCl₃) δ: 8.01 (d, 1H), 7.92 (s, 1H), 7.74 (s, 1H), 7.62 (s, 1H), 6.79 (d, 1H), 6.57 (s, 1H), 6.22-6.51 (m, 1H), 4.16-4.25 (m, 2H), 3.96 (s, 3H), 3.43-3.46 (m, 1H), 1.29-1.32 (m, 1H), 0.85-0.99 (m, 4H).

### Example 154

### (3R,4S)-3-cyclopropyl-1-(6-(1-(2,2-difluorocyclopropyl)-1H-pyrazol-4-yl)pyrrolo[1,2-b]pyridazin-4-yl)-4-methyl-2-oxopyrrolidine-3-carbonitrile

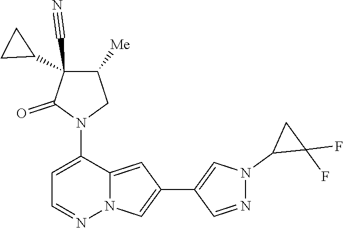 [SMILES: C[C@@H]1CN(c2ccnn3cc(-c4cnn(C5CC5(F)F)c4)cc23)C(=O)[C@]1(C#N)C1CC1]

To a solution of (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 70.0 mg, 0.172 mmol), 4-bromo-1-(2,2-difluorocyclopropyl)-1H-pyrazole (Preparation 137, 96.1 mg, 0.431 mmol), K₂CO₃ (47.7 mg, 0.345 mmol) in dioxane (2 mL) and H₂O (0.2 mL) was added Pd(dppf)Cl₂ (12.7 mg, 0.017 mmol) at 25° C. and the mixture stirred at 70° C. for 2 h under N₂. The mixture was quenched with H₂O (50 mL) and extracted with EtOAc (3×20 mL). The combined organics were washed with brine (20 mL), dried (Na₂SO₄) and evaporated to dryness in vacuo. The residue was purified by prep-HPLC-3 (Gradient: 38-58%) to give (3R,4S)-3-cyclopropyl-1-(6-(1-(2,2-difluorocyclopropyl)-1H-pyrazol-4-yl)pyrrolo[1,2-b]pyridazin-4-yl)-4-methyl-2-oxopyrrolidine-3-carbonitrile as a white solid (14 mg, 19%). LCMS m/z=423.1 [M+H]⁺; ¹H NMR (500 MHz, CDCl₃) δ: 7.99 (d, 1H), 7.91 (s, 1H), 7.80 (s, 1H), 7.76 (s, 1H), 6.85 (d, 1H), 6.60 (s, 1H), 4.09-4.15 (m, 2H), 3.84-3.88 (m, 1H), 3.06-3.12 (m, 1H), 2.24-2.30 (m, 1H), 2.11-2.14 (m, 1H), 1.44 (d, 3H), 1.16-1.19 (m, 1H), 0.76-0.83 (m, 4H).

### Example 155 and 156

### (3R,4S)-3-cyclopropyl-1-(6-(1-((R)-2,2-difluorocyclopropyl)-1H-pyrazol-4-yl)pyrrolo[1,2-b]pyridazin-4-yl)-4-methyl-2-oxopyrrolidine-3-carbonitrile and (3R,4S)-3-cyclopropyl-1-(6-(1-((S)-2,2-difluorocyclopropyl)-1H-pyrazol-4-yl)pyrrolo[1,2-b]pyridazin-4-yl)-4-methyl-2-oxopyrrolidine-3-carbonitrile

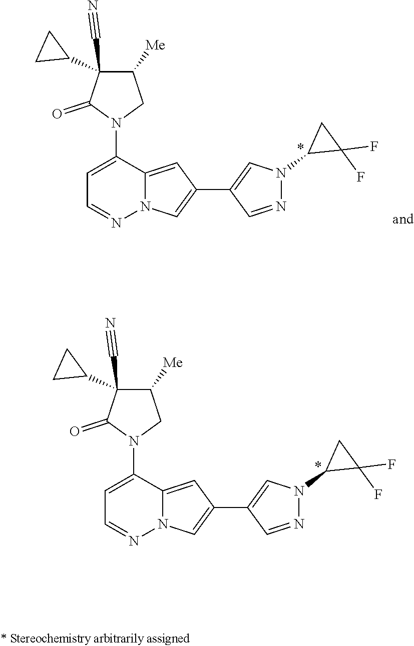 [SMILES: C[C@@H]1CN(c2ccnn3cc(-c4cnn([C@@H]5CC5(F)F)c4)cc23)C(=O)[C@]1(C#N)C1CC1 ; C[C@@H]1CN(c2ccnn3cc(-c4cnn([C@H]5CC5(F)F)c4)cc23)C(=O)[C@]1(C#N)C1CC1]

(3R,4S)-3-cyclopropyl-1-(6-(1-((RS)-2,2-difluorocyclopropyl)-1H-pyrazol-4-yl)pyrrolo[1,2-b]pyridazin-4-yl)-4-methyl-2-oxopyrrolidine-3-carbonitrile (Example 154, 90 mg) was purified by SFC (column: DAICEL CHIRALPAK AD (250 mm×30 mm, 10 um), 0.1% NH₃H₂O IPA, Begin B 25, End B 25, Flow Rate (mL/min): 60, Column temp: 35° C.) to give the title compounds.

*Peak 1, Example 155; (3R,4S)-3-cyclopropyl-1-(6-(1-((R)-2,2-difluorocyclopropyl)-1H-pyrazol-4-yl)pyrrolo[1,2-b]pyridazin-4-yl)-4-methyl-2-oxopyrrolidine-3-carbonitrile as a yellow solid (25.8 mg, 28%); LCMS m/z=423.3 [M+H]⁺; ¹H NMR (400 MHz, DMSO-d₆) δ: 8.30 (s, 1H), 8.23 (s, 1H), 8.14 (d, 1H), 7.97 (s, 1H), 6.94 (d, 2H), 4.62-4.57 (m, 1H), 4.13-4.01 (m, 2H), 3.18-3.09 (m, 1H), 2.45-2.32 (m, 2H), 1.52-1.46 (m, 1H), 1.31 (d, 3H), 0.76-0.67 (m, 2H), 0.58-0.50 (m, 2H).

*Peak 2, Example 156; (3R,4S)-3-cyclopropyl-1-(6-(1-((S)-2,2-difluorocyclopropyl)-1H-pyrazol-4-yl)pyrrolo[1,2-b]pyridazin-4-yl)-4-methyl-2-oxopyrrolidine-3-carbonitrile as a yellow solid (26 mg, 29%); LCMS m/z=423.3 [M+H]⁺; ¹H NMR (400 MHz, DMSO-d₆) δ: 8.30 (s, 1H), 8.23 (s, 1H), 8.14 (d, 1H), 7.97 (s, 1H), 6.93 (d, 2H), 4.62-4.55 (m, 1H), 4.12-4.00 (m, 2H), 3.18-3.09 (m, 1H), 2.46-2.32 (m, 2H), 1.52-1.45 (m, 1H), 1.31 (d, 3H), 0.76-0.69 (m, 2H), 0.57-0.52 (m, 2H).

### Example 157-160

The compounds in the following table were prepared from the (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69) and the appropriate bromide following the procedure described in Example 154.

| Example | |
|---|---|
| No | Name/Structure/RBY/Data |
| 157 | (3R,4S)-3-cyclopropyl-1-(6-(1-(3,3-difluorocyclobutyl)-1H-pyrazol-4- |
| | yl)pyrrolo[1,2-b]pyridazin-4-yl)-4-methyl-2-oxopyrrolidine-3-carbonitrile |
| | 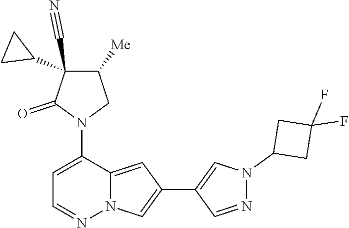 [SMILES: C[C@@H]1CN(c2ccnn3cc(-c4cnn(C5CC(F)(F)C5)c4)cc23)C(=O)[C@]1(C#N)C1CC1] |
| | RBr: 4-bromo-1-(3,3-difluorocyclobutyl)-1H-pyrazole (Preparation 139) |
| | HPLC-3 (Gradient: 45-75%); 23 mg, 57% as yellow solid. LCMS m/z = |
| | 437.2 [M + H]⁺; ¹H NMR (500 MHz, CDCl₃) δ: 7.99 (d, 1H), 7.91 (s, 1H), |
| | 7.81 (s, 1H), 7.70 (s, 1H), 6.83 (d, 1H), 6.60 (s, 1H), 4.78-4.74 (m, 1H), |
| | 4.13-4.09 (m, 1H), 3.88-3.84 (m, 1H), 3.28-3.15 (m, 5H), 1.45 (d, 3H), |
| | 1.22-1.16 (m, 1H), 0.83-0.76 (m, 4H). |
| 158 | (3R,4S)-3-cyclopropyl-4-methyl-2-oxo-1-(6-(1-(pyridin-2-yl)-1H-pyrazol- |
| | 4-yl)pyrrolo[1,2-b]pyridazin-4-yl)pyrrolidine-3-carbonitrile |
| | 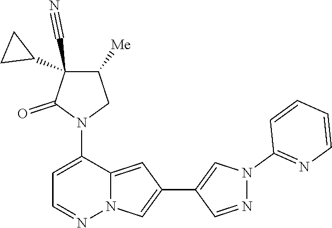 [SMILES: C[C@@H]1CN(c2ccnn3cc(-c4cnn(-c5ccccn5)c4)cc23)C(=O)[C@]1(C#N)C1CC1] |
| | RBr: 2-(4-bromo-1H-pyrazol-1-yl)pyridine |
| | HPLC-3 (Gradient: 47-77%); 48 mg, 46% as yellow solid. LCMS m/z = |
| | 424.3 [M + H]⁺; ¹H NMR (500 MHz, DMSO-d₆) δ: 9.13 (s, 1H), 8.51 (d, |
| | 1H), 8.44 (d, 1H), 8.31 (s, 1H), 8.18 (d, 1H), 8.03-8.01 (m, 1H), 7.99-7.97 |
| | (m, 1H), 7.39 (d, 1H), 7.19 (d, 1H), 7.00 (d, 1H), 4.15-4.12 (m, 2H), 3.19- |
| | 3.14 (m, 1H), 1.54-1.52 (m, 1H), 1.34 (d, 3H), 0.77-0.71 (m, 2H), 0.58-0.55 |
| | (m, 2H). |
| 159 | (3R,4S)-3-cyclopropyl-4-methyl-2-oxo-1-(6-(1-(pyridin-3-yl)-1H-pyrazol- |
| | 4-yl)pyrrolo[1,2-b]pyridazin-4-yl)pyrrolidine-3-carbonitrile |
| | 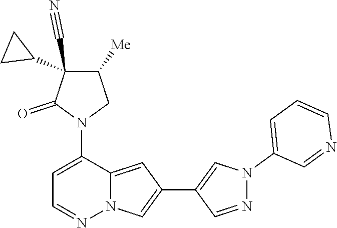 [SMILES: C[C@@H]1CN(c2ccnn3cc(-c4cnn(-c5cccnc5)c4)cc23)C(=O)[C@]1(C#N)C1CC1] |
| | RBr: 3-(4-bromo-1H-pyrazol-1-yl)pyridine (Preparation 140) |
| | HPLC-3 (Gradient: 40-70%); 50 mg, 24% as yellow solid. LCMS m/z = |
| | 424.1 [M + H]⁺; ¹H NMR (500 MHz, DMSO-d₆) δ: 9.15 (d1H), 9.04 (s, 1H), |
| | 8.56-8.54 (m, 1H), 8.34 (d, 1H), 8.29-8.28 (m, 2H), 8.19 (d, 1H), 7.60-7.58 |
| | (m, 1H), 7.03 (d, 1H), 6.98 (d, 1H), 4.16-4.12 (m, 1H), 4.08-4.04 (m, 1H), |
| | 3.19-3.14 (m, 1H), 1.53-1.51 (m, 1H), 1.34 (d, 3H), 0.77-0.72 (m, 2H), |
| | 0.58-0.55 (m, 2H). |
| 160 | (3R,4S)-3-cyclopropyl-4-methyl-2-oxo-1-(6-(1-(pyridin-4-yl)-1H-pyrazol- |
| | 4-yl)pyrrolo[1,2-b]pyridazin-4-yl)pyrrolidine-3-carbonitrile |
| | 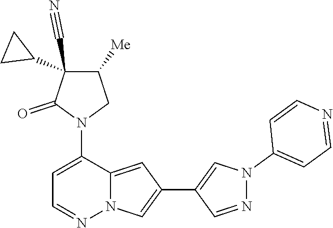 [SMILES: C[C@@H]1CN(c2ccnn3cc(-c4cnn(-c5ccncc5)c4)cc23)C(=O)[C@]1(C#N)C1CC1] |
| | RBr: 4-(4-bromo-1H-pyrazol-1-yl)pyridine |
| | HPLC-11 (Gradient, 20-40%); 65 mg, 34% as yellow solid. LCMS m/z = |
| | 424.3 [M + H]⁺; ¹H NMR (400 MHz, DMSO-d₆) δ: 9.12 (s, 1H), 8.67 (d, |
| | 2H), 8.34 (d, 2H), 8.19 (d, 1H), 7.87 (d, 2H), 7.03 (s, 1H), 6.98 (d, 1H), |
| | 4.13 (t, 1H), 4.05 (t, 1H), 3.17-3.11 (m, 1H), 1.53-1.47 (m, 1H), 1.32 (d, |
| | 3H), 0.78-0.68 (m, 2H), 0.59-0.51 (m, 2H). |

### Example 161-163

The compounds in the following table were prepared from (3R,4S)-1-(6-bromopyrrolo[1,2-b]pyridazin-4-yl)-3-cyclopropyl-4-methyl-2-oxopyrrolidine-3-carbonitrile (Preparation 68) and the appropriate boronic acid or ester (RBY) following the procedure described in Example 8.

| Example | |
|---|---|
| No | Name/Structure/RBY/Data |
| 161 | (3R,4S)-3-cyclopropyl-1-(6-(1-cyclopropyl-1H-pyrazol-4-yl)pyrrolo[1,2- |
| | b]pyridazin-4-yl)-4-methyl-2-oxopyrrolidine-3-carbonitrile |
| | 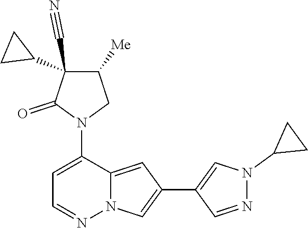 [SMILES: C[C@@H]1CN(c2ccnn3cc(-c4cnn(C5CC5)c4)cc23)C(=O)[C@]1(C#N)C1CC1] |
| | RBY: 1-cyclopropyl-4-(4,4,5,5-tetramethyl-1,3,2-dioxaborolan-2-yl)-1H- |
| | pyrazole |
| | HPLC-3 (Gradient: 32-62%); 40 mg, 37% as yellow solid. LCMS m/z = |
| | 387.2 [M + H]⁺; ¹H NMR (400 MHz, CDCl₃) δ: 7.97 (d, 1H), 7.88 (s, 1H), |
| | 7.71-7.69 (m, 2H), 6.83 (d, 1H), 6.58 (s, 1H), 4.13-4.08 (m, 1H), 3.88-3.82 |
| | (m, 1H), 3.64-3.62 (m, 1H), 3.13-3.03 (m, 1H), 1.43 (d, 3H), 1.20-1.13 (m, |
| | 3H), 1.06-1.04 (m, 2H), 0.82-0.76 (m, 4H). |
| 162 | (3R,4S)-3-cyclopropyl-4-methyl-2-oxo-1-(6-(1-(tetrahydrofuran-3-yl)-1H- |
| | pyrazol-4-yl)pyrrolo[1,2-b]pyridazin-4-yl)pyrrolidine-3-carbonitrile |
| | 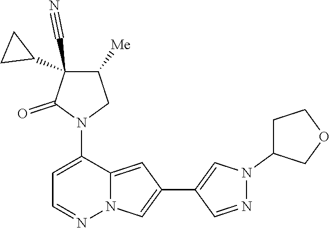 [SMILES: C[C@@H]1CN(c2ccnn3cc(-c4cnn(C5CCOC5)c4)cc23)C(=O)[C@]1(C#N)C1CC1] |
| | RBY: 1-(tetrahydrofuran-3-yl)-4-(4,4,5,5-tetramethyl-1,3,2-dioxaborolan-2- |
| | yl)-1H-pyrazole |
| | HPLC-3 (Gradient: 32-62%); 69 mg, 60% as green solid. |
| | LCMS m/z = 417.2 [M + H]⁺; ¹H NMR (400 MHz, CDCl₃) δ: 7.98 (d, 1H), |
| | 7.90 (d, 1H), 7.75-7.73 (m, 2H), 6.87-6.85 (m, 1H), 6.59 (d, 1H), 5.05-5.02 |
| | (m, 1H), 4.18-4.10 (m, 4H), 3.97-3.95 (m, 1H), 3.88-3.85 (m, 1H), 3.10- |
| | 3.08 (m, 1H), 2.52-2.49 (m, 1H), 2.38-2.35 (m, 1H), 1.45 (d, 3H), 1.20-1.17 |
| | (m, 1H), 0.82-0.76 (m, 4H). |
| 163 | (3R,4S)-3-cyclopropyl-4-methyl-1-(6-(1-(methylsulfonyl)-1H-pyrazol-4- |
| | yl)pyrrolo[1,2-b]pyridazin-4-yl)-2-oxopyrrolidine-3-carbonitrile |
| | 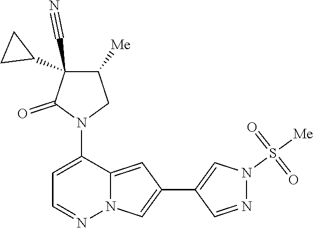 [SMILES: C[C@@H]1CN(c2ccnn3cc(-c4cnn(S(C)(=O)=O)c4)cc23)C(=O)[C@]1(C#N)C1CC1] |
| | RBY: 1-(methylsulfonyl)-4-(4,4,5,5-tetramethyl-1,3,2-dioxaborolan-2-yl)- |
| | 1H-pyrazole (Preparation 141). |
| | HPLC-3 (Gradient: 39-69%). 35 mg, 30 % as yellow solid. |
| | LCMS m/z = 425.1 [M + H]⁺; ¹H NMR (400 MHz, CDCl₃) δ: 8.24 (s, 1H), |
| | 8.06 (s, 1H), 8.03 (d, 1H), 7.96 (s, 1H), 6.88 (d, 1H), 6.54 (s, 1H), 4.14-4.09 |
| | (m, 1H), 3.89-3.84 (m, 1H), 3.39 (s, 3H), 3.13-3.07 (m, 1H), 1.45 (d, 3H), |
| | 1.97-1.51 (m, 1H), 0.83-0.78 (m, 4H). |

### Example 164

### (3R,4 S)-3-cyclopropyl-4-methyl-1-(6-(1-(methyl-d3)-1H-pyrazol-4-yl)pyrrolo[1,2-b]pyridazin-4-yl)-2-oxopyrrolidine-3-carbonitrile

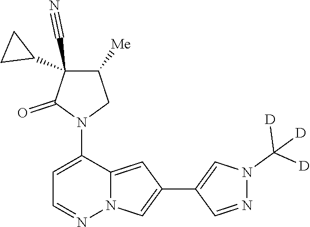 [SMILES: [2H]C([2H])([2H])n1cc(-c2cc3c(N4C[C@@H](C)[C@@](C#N)(C5CC5)C4=O)ccnn3c2)cn1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 100 mg, 0.278 mmol) in dioxane (2 mL) and water (0.2 mL) was added 1-(methyl-d3)-4-(4,4,5,5-tetramethyl-1,3,2-dioxaborolan-2-yl)-1H-pyrazole (Preparation 117, 88.1 mg, 0.418 mmol), KF (3.0 M, 0.28 mL) and Pd(dppf)Cl₂ (20.4 mg, 0.028 mmol) under N₂ and the mixture was stirred at 70° C. overnight. EtOAc was added and washed with H₂O and brine. The combined organics were dried (Na₂SO₄) and evaporated to dryness in vacuo. The residue was purified by reverse phase chromatography to afford (3R,4S)-3-cyclopropyl-4-methyl-1-(6-(1-(methyl-d3)-1H-pyrazol-4-yl)pyrrolo[1,2-b]pyridazin-4-yl)-2-oxopyrrolidine-3-carbonitrile as a black oil (22.7 mg). LCMS m/z=364 [M+H]⁺; ¹H NMR (600 MHz, DMSO-d₆) δ: 8.19-8.16 (m, 1H), 8.14-8.10 (m, 1H), 8.07-8.04 (m, 1H), 7.83-7.81 (m, 1H), 6.92-6.90 (m, 1H), 6.87-6.86 (m, 1H), 4.14-4.98 (m, 1H), 4.04-3.98 (m, 1H), 3.20-3.08 (m, 1H), 1.55-1.46 (m, 1H), 1.33-1.28 (m, 3H), 0.78-0.66 (m, 2H), 0.61-0.51 (m, 2H).

### Example 165

### (3R,4S)-3-cyclopropyl-1-(6-(1,3-dimethyl-1H-pyrazol-4-yl)pyrrolo[1,2-b]pyridazin-4-yl)-4-methyl-2-oxopyrrolidine-3-carbonitrile

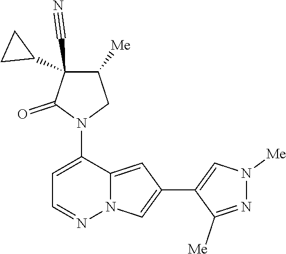 [SMILES: Cc1nn(C)cc1-c1cc2c(N3C[C@@H](C)[C@@](C#N)(C4CC4)C3=O)ccnn2c1]

A mixture of (3R,4S)-1-(6-bromopyrrolo[1,2-b]pyridazin-4-yl)-3-cyclopropyl-4-methyl-2-oxopyrrolidine-3-carbonitrile (Preparation 68, 100 mg, 0.278 mmol), 1,3-dimethyl-4-(4,4,5,5-tetramethyl-1,3,2-dioxaborolan-2-yl)pyrazole (124 mg, 0.557 mmol), Pd(dppf)Cl₂ (20.4 mg, 0.028 mmol) and K₂CO₃ (115 mg, 0.835 mmol) in dioxane (2 mL) and H₂O (0.2 mL) was stirred at 70° C. overnight. The reaction mixture was diluted with EtOAc, washed with H₂O and brine. The combined organics were dried (Na₂SO₄) and evaporated to dryness in vacuo. The residue was purified by reverse phase chromatography to afford (3R,4S)-3-cyclopropyl-1-(6-(1,3-dimethyl-1H-pyrazol-4-yl)pyrrolo[1,2-b]pyridazin-4-yl)-4-methyl-2-oxopyrrolidine-3-carbonitrile as a black oil (43.4 mg). LCMS m/z=375 [M+H]⁺; ¹H NMR (600 MHz, DMSO-d₆) δ: 8.17-8.13 (m, 1H), 8.07-8.02 (m, 1H), 7.98-7.93 (m, 1H), 6.93-6.88 (m, 1H), 6.78-6.73 (m, 1H), 4.15-4.07 (m, 1H), 4.01-3.94 (m, 1H), 3.81-3.76 (m, 3H), 3.21-3.10 (in, 1H), 2.36-2.32 (m, 3H), 1.53-1.46 (m, 1H), 1.34-1.28 (m, 3H), 0.79-0.66 (m, 2H), 0.51-0.51 (in, 2H).

### Example 166-179

The title compounds were prepared from (3R,4S)-1-(6-bromopyrrolo[1,2-b]pyridazin-4-yl)-3-cyclopropyl-4-methyl-2-oxopyrrolidine-3-carbonitrile (Preparation 68) and the appropriate boronate ester (RBY) using an analogous method to that described for Example 165. Purified using prep-HPLC as described in the table.

| Example No | Name/Structure/RBY/Data |
|---|---|
| 166 | (3R,4S)-3-cyclopropyl-1-(6-(1,5-dimethyl-1H-pyrazol-4-yl)pyrrolo[1,2- |
| | b]pyridazin-4-yl)-4-methyl-2-oxopyrrolidine-3-carbonitrile |
| | 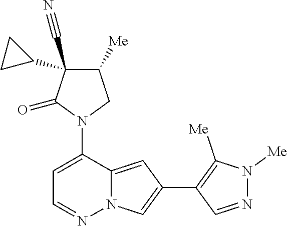 [SMILES: Cc1c(-c2cc3c(N4C[C@@H](C)[C@@](C#N)(C5CC5)C4=O)ccnn3c2)cnn1C] |
| | RBY: 1,5-dimethyl-4-(4,4,5,5-tetramethyl-1,3,2-dioxaborolan-2-yl)-1H- |
| | pyrazole |
| | Prep-HPLC-10 (Gradient: 5-55%); 39.1 mg as beige solid. |
| | LCMS m/z = 375 [M + H]⁺; ¹H NMR (600 MHz, DMSO-d₆) δ: 8.17-8.13 |
| | (m, 1H), 8.10-8.05 (m, 1H), 7.69-7.66 (m, 1H), 6.94-6.90 (m, 1H), 6.80- |
| | 6.77 (m, 1H), 4.15-4.08 (m, 1H), 4.02-3.95 (m, 1H), 3.81-3.75 (m, 3H), |
| | 3.19-3.12 (m, 1H), 2.43 (s, 3H), 1.55-1.48 (m, 1H), 1.34-1.28 (m, 3H), |
| | 0.79-0.65, (m, 2H), 0.60-0.50 (m, 2H). |
| 167 | (3R,4S)-3-cyclopropyl-1-(6-(1-isopropyl-1H-pyrazol-4-yl)pyrrolo[1,2- |
| | b]pyridazin-4-yl)-4-methyl-2-oxopyrrolidine-3-carbonitrile |
| | 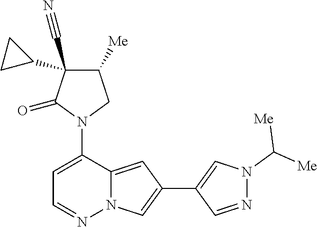 [SMILES: CC(C)n1cc(-c2cc3c(N4C[C@@H](C)[C@@](C#N)(C5CC5)C4=O)ccnn3c2)cn1] |
| | RBY: 1-isopropyl-4-(4,4,5,5-tetramethyl-1,3,2-dioxaborolan-2-yl)-1H- |
| | pyrazole |
| | prep-HPLC-10 (Gradient: 5-65%); 41.8 mg as black oil. |
| | LCMS m/z = 389 [M + H]⁺; ¹H NMR (600 MHz, DMSO-d₆) δ: 8.19-8.17 |
| | (m, 1H), 8.15-8.13 (m, 1H), 8.13-8.10 (m, 1H), 7.84-7.81 (m, 1H), 6.93- |
| | 6.90 (m, 1H), 6.90-6.83 (m, 1H), 4.55-4.45 (m, 1H), 4.14-4.07 (m, 1H), |
| | 4.06-3.98 (m, 1H), 3.21-3.05 (m, 1H), 1.53-1.48 (m, 1H), 1.48-1.40 (m, |
| | 6H), 1.34-1.29 (m, 3H), 0.78-0.65 (m, 2H), 0.60-0.50 (m, 2H). |
| 168 | (3R,4S)-1-(6-(1-(cyanomethyl)-1H-pyrazol-4-yl)pyrrolo[1,2-b]pyridazin-4- |
| | yl)-3-cyclopropyl-4-methyl-2-oxopyrrolidine-3-carbonitrile |
| | 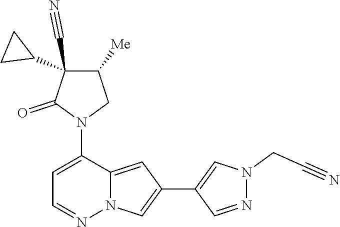 [SMILES: C[C@@H]1CN(c2ccnn3cc(-c4cnn(CC#N)c4)cc23)C(=O)[C@]1(C#N)C1CC1] |
| | RBY: 2-(4-(4,4,5,5-tetramethyl-1,3,2-dioxaborolan-2-yl)-1H-pyrazol-1- |
| | yl)acetonitrile |
| | HPLC-9 (Gradient: 5-60%); 61.8 mg as dark brown oil. |
| | LCMS m/z = 386 [M + H]⁺; ¹H NMR (600 MHz, DMSO-d₆) δ: 8.26-8.23 |
| | (m, 1H), 8.22-8.20 (m, 1H), 8.16-8.12 (m, 1H), 8.03-8.00 (m, 1H), 6.95- |
| | 6.91 (m, 1H), 6.91-6.87 (m, 1H), 5.52-5.47 (m, 2H), 4.14-4.07 (m, 1H), |
| | 4,04-3.97 (m, 1H), 3.19-3.06 (m, 1H), 1.53-1.43 (m, 1H), 1.32-1.28 (m, |
| | 3H), 0.78-0.65 (m, 2H), 0.59-0.48 (m, 2H). |
| 169 | (3R,4S)-1-(6-(1-cyclohexyl-1H-pyrazol-4-yl)pyrrolo[1,2-b]pyridazin-4-yl)- |
| | 3-cyclopropyl-4-methyl-2-oxopyrrolidine-3-carbonitrile |
| | 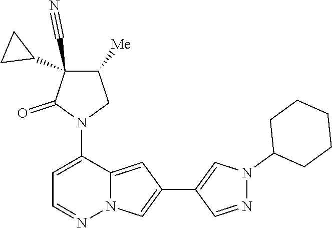 [SMILES: C[C@@H]1CN(c2ccnn3cc(-c4cnn(C5CCCCC5)c4)cc23)C(=O)[C@]1(C#N)C1CC1] |
| | RBY: 1-cyclohexyl-4-(4,4,5,5-tetramethyl-1,3,2-dioxaborolan-2-yl)-1H- |
| | pyrazole |
| | HPLC-9 (Gradient: 20-75%); 56.7 mg as dark brown oil. |
| | LCMS m/z = 429 [M + H]⁺; ¹H NMR (600 MHz, DMSO-d₆) δ: 8.19-8.16 |
| | (m, 1H), 8.15-8.13 (m, 1H), 8.13-8.09 (m, 1H), 7.84-7.80 (m, 1H), 6.92- |
| | 6.89 (m, 1H), 6.88-6.84 (m, 1H), 4.17-4.11 (m, 1H), 4.11-4.05 (m, 1H), |
| | 4.03-3.97 (m, 1H), 3.21-3.06 (m, 1H), 2.09-2.02 (m, 2H), 1.87-1.80 (m, |
| | 2H), 1.78-1.60 (m, 3H), 1.53-1.45 (m, 1H), 1.44-1.35 (m, 2H), 1.34-1.26 |
| | (m, 3H), 1.26-1.19 (m, 1H), 0.80-0.62 (m, 2H), 0.60-0.45 (m, 2H). |
| 170 | (3R,4S)-1-(6-(1-cyclopentyl-1H-pyrazol-4-yl)pyrrolo[1,2-b]pyridazin-4- |
| | yl)-3-cyclopropyl-4-methyl-2-oxopyrrolidine-3-carbonitrile |
| | 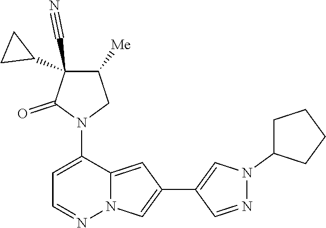 [SMILES: C[C@@H]1CN(c2ccnn3cc(-c4cnn(C5CCCC5)c4)cc23)C(=O)[C@]1(C#N)C1CC1] |
| | RBY: 1-cyclopentyl-4-(4,4,5,5-tetramethyl-1,3,2-dioxaborolan-2-yl)-1H- |
| | pyrazole |
| | HPLC-9 (Gradient: 5-70%); 80.7 mg as dark brown oil. |
| | LCMS m/z = 415 [M + H]⁺; ¹H NMR (600 MHz, DMSO-d₆) δ: 8.17-8.20 |
| | (m, 1H), 8.13-8.15 (m, 1H), 8.09-8.13 (m, 1H), 7.81-7.84 (m, 1H), 6.88- |
| | 6.92 (m, 1H), 6.85-6.88 (m, 1H), 4.64-4.73 (m, 1H), 4.06-4.12 (m, 1H), |
| | 3.96-4.03 (m, 1H), 3.08-3.19 (m, 1H), 2.03-2.14 (m, 2H), 1.88-1.99 (m, |
| | 2H), 1.74-1.85 (m, 2H), 1.59-1.71 (m, 2H), 1.44-1.51 (m, 1H), 1.28-1.34 |
| | (m, 3H), 0.64-0.78 (m, 2H), 0.49-0.60 (m, 2H). |
| 171 | (3R,4S)-3-cyclopropyl-1-(6-(5,6-dihydro-4H-pyrrolo[1,2-b]pyrazol-3- |
| | yl)pyrrolo[1,2-b]pyridazin-4-yl)-4-methyl-2-oxopyrrolidine-3-carbonitrile |
| | 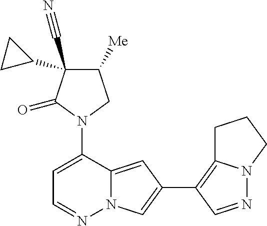 [SMILES: C[C@@H]1CN(c2ccnn3cc(-c4cnn5c4CCC5)cc23)C(=O)[C@]1(C#N)C1CC1] |
| | RBY: 3-(4,4,5,5-tetramethyl-1,3,2-dioxaborolan-2-yl)-5,6-dihydro-4H- |
| | pyrrolo[1,2-b]pyrazole |
| | HPLC-9 (Gradient: 5-60%); 74.3 mg dark brown oil. |
| | LCMS m/z = 387 [M + H]⁺; ¹H NMR (600 MHz, DMSO-d₆) δ: 8.15-8.11 |
| | (m, 1H), 8.08-8.05 (m, 1H), 7.85-7.82 (m, 1H), 6.91-6.87 (m, 1H), 6.76- |
| | 6.73 (m, 1H), 4.14-3.92 (m, 4H), 3.46-3.44 (m, 2H), 3.05-3.02 (m, 1H), |
| | 2.66-2.58 (m, 2H), 1.33-1.26 (m, 3H), 0.78-0.66 (m, 2H), 0.59-0.50 (m, |
| | 2H). |
| 172 | (3R,4S)-3-cyclopropyl-1-(6-(1-(2-ethoxyethyl)-1H-pyrazol-4- |
| | yl)pyrrolo[1,2-b]pyridazin-4-yl)-4-methyl-2-oxopyrrolidine-3-carbonitrile |
| | 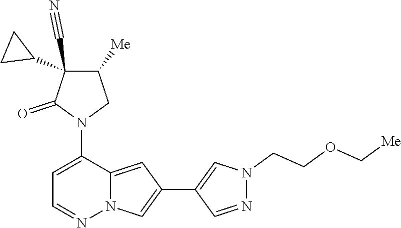 [SMILES: CCOCCn1cc(-c2cc3c(N4C[C@@H](C)[C@@](C#N)(C5CC5)C4=O)ccnn3c2)cn1] |
| | RBY: 1-(2-ethoxyethyl)-4-(4,4,5,5-tetramethyl-1,3,2-dioxaborolan-2-yl)- |
| | 1H-pyrazole (Preparation 142) |
| | HPLC-9 (Gradient: 5-65%); 2.8 mg as brown oil. |
| | LCMS m/z = 419 [M + H]⁺; ¹H NMR (600 MHz, DMSO-d₆) δ: 8.21-8.11 |
| | (m, 1H), 8.10-7.80 (m, 1H), 7.66-7.61 (m, 1H), 7.46-7.37 (m, 1H), 6.94- |
| | 6.90 (m, 1H), 6.89-6.82 (m, 1H), 5.62-5.60 (m, 1H), 4.30-4.18 (m, 2H), |
| | 4.13-4.07 (m, 1H), 4.05-3.98 (m, 1H), 3.79-3.67 (m, 2H), 1.55-1.46 (m, |
| | 1H), 1.38-1.21 (m, 5H), 1.15-1.02 (m, 3H), 0.78-0.65 (m, 2H), 0.59-0.49 |
| | (m, 2H). |
| 173 | (3R,4S)-3-cyclopropyl-4-methyl-2-oxo-1-(6-(4,5,6,7- |
| | tetrahydropyrazolo[1,5-a]pyridin-3-yl)pyrrolo[1,2-b]pyridazin-4- |
| | yl)pyrrolidine-3-carbonitrile |
| | 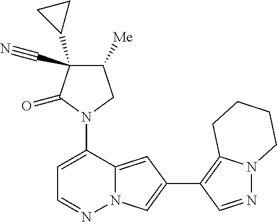 [SMILES: C[C@@H]1CN(c2ccnn3cc(-c4cnn5c4CCCC5)cc23)C(=O)[C@]1(C#N)C1CC1] |
| | RBY: 3-(4,4,5,5-tetramethyl-1,3,2-dioxaborolan-2-yl)-4,5,6,7- |
| | tetrahydropyrazolo[1,5-a]pyridine |
| | HPLC-9 (Gradient: 5-60%); 24 mg as brown oil. |
| | LCMS m/z = 401 [M + H]⁺; ¹H NMR (600 MHz, DMSO-d₆) δ: 8.16-8.12 |
| | (m, 1H), 8.08-8.05 (m, 1H), 7.80-7.76 (m, 1H), 6.92-6.89 (m, 1H), 6.81- |
| | 6.76 (m, 1H), 4.19-4.06 (m, 3H), 4.03-3.96 (m, 1H), 2.97-2.91 (m, 1H), |
| | 2.08-1.95 (m, 2H), 1.92-1.81 (m, 2H), 1.55-1.48 (m, 1H), 1.32-1.29 (m, |
| | 3H), 1.28-1.23 (m, 2H), 0.78-0.65 (m, 2H), 0.60-0.51 (m, 2H). |
| 174 | (3R,4S)-3-cyclopropyl-1-(6-(6-ethylpyridin-3-yl)pyrrolo[1,2-b]pyridazin-4- |
| | yl)-4-methyl-2-oxopyrrolidine-3-carbonitrile trifluoroacetate |
| | 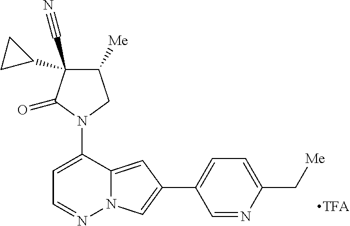 [SMILES: CCc1ccc(-c2cc3c(N4C[C@@H](C)[C@@](C#N)(C5CC5)C4=O)ccnn3c2)cn1] |
| | RBY: 2-ethyl-5-(4,4,5,5-tetramethyl-1,3,2-dioxaborolan-2-yl)pyridine |
| | HPLC-9 (Gradient: 5-50%); 54.2 mg as brown oil. |
| | LCMS m/z = 386 [M + H]⁺; ¹H NMR (600 MHz, DMSO-d₆) δ: 9.05 (s, 1H), |
| | 8.64-8.58 (m, 1H), 8.45-8.34 (m, 1H), 8.25-8.18 (m, 1H), 7.61-7.50 (m, |
| | 1H), 7.28-7.23 (m, 1H), 7.02-7.00 (m, 1H), 4.18-4.12 (m, 1H), 4.11-4.07 |
| | (m, 1H), 2.92-2.76 (m, 2H), 1.55-1.48 (m, 1H), 1.35-1.31 (m, 3H), 1.30- |
| | 1.26 (m, 3H), 0.82-0.65 (m, 2H), 0.62-0.48 (m, 2H). |
| 175 | (3R,4S)-3-cyclopropyl-1-(6-(4-ethylpyridin-3-yl)pyrrolo[1,2-b]pyridazin-4- |
| | yl)-4-methyl-2-oxopyrrolidine-3-carbonitrile trifluoroacetate |
| | 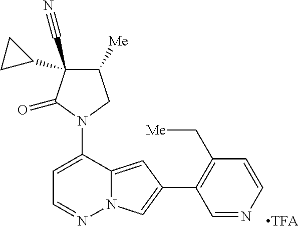 [SMILES: CCc1ccncc1-c1cc2c(N3C[C@@H](C)[C@@](C#N)(C4CC4)C3=O)ccnn2c1] |
| | RBY: 4-ethyl-3-(4,4,5,5-tetramethyl-1,3,2-dioxaborolan-2-yl)pyridine |
| | HPLC-9 (Gradient: 5-50%); 49.9 mg as yellow oil. |
| | LCMS m/z = 386 [M + H]⁺; ¹H NMR (600 MHz, DMSO-d₆) δ: 8.75-8.66 |
| | (m, 1H), 8.62-8.55 (m, 1H), 8.28-8.26 (m, 1H), 8.26-8.23 (m, 1H), 7.67- |
| | 7.52 (m, 1H), 7.05-7.02 (m, 1H), 6.97-6.94 (m, 1H), 4.18-4.12 (m, 1H), |
| | 4.06-4.01 (m, 1H), 2.94-2.86 (m, 2H), 1.54-1.47 (m, 1H), 1.32-1.28 (m, |
| | 3H), 1.20-1.15 (m, 3H), 0.77-0.64 (m, 2H), 0.59-0.50 (m, 2H). |
| 176 | (3R,4S)-3-cyclopropyl-1-(6-(6-(difluoromethoxy)pyridin-3-yl)pyrrolo[1,2- |
| | b]pyridazin-4-yl)-4-methyl-2-oxopyrrolidine-3-carbonitrile |
| | 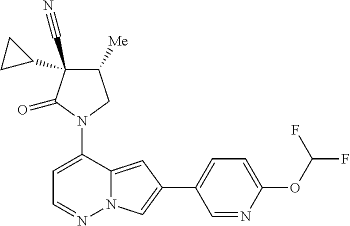 [SMILES: C[C@@H]1CN(c2ccnn3cc(-c4ccc(OC(F)F)nc4)cc23)C(=O)[C@]1(C#N)C1CC1] |
| | RBY: 2-(difluoromethoxy)-5-(4,4,5,5-tetramethyl-1,3,2-dioxaborolan-2- |
| | yl)pyridine |
| | HPLC-9 (Gradient: 20-75%); 15.9 mg as off-white solid. |
| | LCMS m/z = 424 [M + H]⁺; ¹H NMR (600 MHz, DMSO-d₆) δ: 8.82-8.75 |
| | (m, 1H), 8.57-8.50 (m, 1H), 8.40-8.31 (m, 1H), 8.25-8.17 (m, 1H), 7.90- |
| | 7.57 (m, 1H), 7.22-7.20 (m, 1H), 7.18-7.14 (m, 1H), 7.03-6.98 (m, 1H), |
| | 4.18-4.12 (m, 1H), 4.12-4.06 (m, 1H), 1.54-1.46 (m, 1H), 1.32 (d, 3H), |
| | 0.81-0.64 (m, 2H), 0.60-0.48 (m, 2H). |
| 177 | (3R,4S)-3-cyclopropyl-1-(6-(5-(difluoromethoxy)pyridin-3-yl)pyrrolo[1,2- |
| | b]pyridazin-4-yl)-4-methyl-2-oxopyrrolidine-3-carbonitrile |
| | 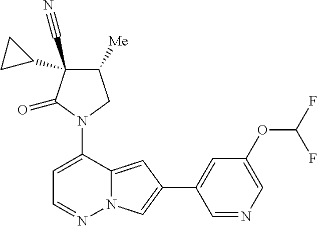 [SMILES: C[C@@H]1CN(c2ccnn3cc(-c4cncc(OC(F)F)c4)cc23)C(=O)[C@]1(C#N)C1CC1] |
| | RBY: 3-(difluoromethoxy)-5-(4,4,5,5-tetramethyl-1,3,2-dioxaborolan-2- |
| | yl)pyridine |
| | HPLC-9 (Gradient: 5-65%); 36.7 mg as off-white solid. |
| | LCMS m/z = 424 [M + H]⁺; ¹H NMR (600 MHz, DMSO-d₆) δ: 9.07-8.99 |
| | (m, 1H), 8.67-8.62 (m, 1H), 8.44-8.36 (m, 1H), 8.26-8.18 (m, 1H), 8.16- |
| | 8.05 (m, 1H), 7.59-7.23 (m, 2H), 7.06-7.00 (m, 1H), 4.20-4.09 (m, 2H), |
| | 1.55-1.47 (m, 1H), 1.35-1.30 (m, 3H), 0.79-0.65 (m, 2H), 0.61-0.50 (m, 2H) |
| 178 | (3R,4S)-3-cyclopropyl-1-(6-(2-ethylpyridin-3-yl)pyrrolo[1,2-b]pyridazin-4- |
| | yl)-4-methyl-2-oxopyrrolidine-3-carbonitrile trifluoroacetate |
| | 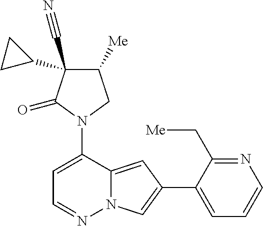 [SMILES: CCc1ncccc1-c1cc2c(N3C[C@@H](C)[C@@](C#N)(C4CC4)C3=O)ccnn2c1] |
| | RBY: 2-ethyl-3-(4,4,5,5-tetramethyl-1,3,2-dioxaborolan-2-yl)pyridine |
| | HPLC-9 (Gradient: 5-50%); 40.8 mg as off-white solid. |
| | LCMS m/z = 386 [M + H]⁺; ¹H NMR (600 MHz, DMSO-d₆) δ: 8.60-8.55 |
| | (m, 1H), 8.29-8.25 (m, 1H), 8.24-8.20 (m, 1H), 8.03-7.95 (m, 1H), 7.51- |
| | 7.44 (m, 1H), 7.03-7.0 (m, 1H), 6.93-6.90 (m, 1H), 4.18-4.11 (m, 1H), 4.07- |
| | 4.00 (m, 1H), 3.19-2.97 (m, 2H), 1.55-1.46 (m, 1H), 1.31-1.27 (m, 3H), |
| | 1.25-1.24 (m, 1H), 1.25-1.20 (m, 2H), 0.76-0.64 (m, 2H), 0.59-0.50 (m, |
| | 2H). |
| 179 | (3R,4S)-3-cyclopropyl-4-methyl-2-oxo-1-(6-(4-(trifluoromethyl)pyridin-3- |
| | yl)pyrrolo[1,2-b]pyridazin-4-yl)pyrrolidine-3-carbonitrile |
| | 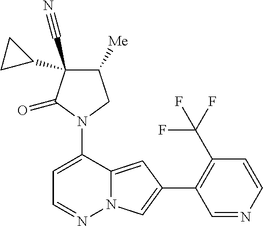 [SMILES: C[C@@H]1CN(c2ccnn3cc(-c4cnccc4C(F)(F)F)cc23)C(=O)[C@]1(C#N)C1CC1] |
| | RBY: 3-(4,4,5,5-tetramethyl-1,3,2-dioxaborolan-2-yl)-4- |
| | trifluoromethylpyridine |
| | HPLC-9 (Gradient: 5-70%); 43.4 mg as brown oil. |
| | LCMS m/z = 426 [M + H]⁺; ¹H NMR (600 MHz, DMSO-d₆) δ: 8.95-8.90 |
| | (m, 1H), 8.87-8.83 (m, 1H), 8.32-8.26 (m, 1H), 8.19-8.15 (m, 1H), 7.89- |
| | 7.83 (m, 1H), 7.06-7.03 (m, 1H), 6.96 (s, 1H), 4.19-4.12 (m, 1H), 4.03 (t, |
| | 1H), 1.54-1.46 (m, 1H), 1.31-1.27 (m, 3H), 0.76-0.62 (m, 2H), 0.59-0.49 |
| | (m, 2H). |

### Example 180

### (3R,4S)-3-cyclopropyl-4-methyl-1-(6-(1-methyl-1H-pyrazol-4-yl)pyrrolo[2,1-f][1,2,4]triazin-4-yl)-2-oxopyrrolidine-3-carbonitrile

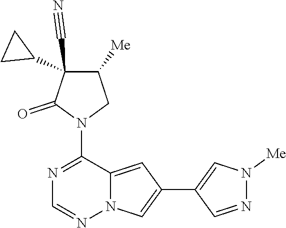 [SMILES: C[C@@H]1CN(c2ncnn3cc(-c4cnn(C)c4)cc23)C(=O)[C@]1(C#N)C1CC1]

(3R,4S)-3-Cyclopropyl-4-methyl-1-(6-(1-methyl-1H-pyrazol-4-yl)pyrrolo[2,1-f][1,2,4]triazin-4-yl)-2-oxopyrrolidine-3-carbonitrile was obtained as a white solid, 20 mg, 49.8% yield, from (3R,4S)-1-(6-bromopyrrolo[2,1-f][1,2,4]triazin-4-yl)-3-cyclopropyl-4-methyl-2-oxopyrrolidine-3-carbonitrile (Preparation 99) and 1-methyl-4-(4,4,5,5-tetramethyl-1,3,2-dioxaborolan-2-yl)-1H-pyrazole, following the procedure described in Example 8. LCMS m/z=362.2 [M+H]+; 1H NMR (500 MHz, CDCl3) δ: 8.13 (s, 1H), 7.95 (s, 1H), 7.75 (s, 1H), 7.67 (s, 1H), 7.39 (s, 1H), 4.24-4.19 (m, 1H), 3.95 (s, 3H), 3.86 (t, 1H), 3.08-3.03 (m, 1H), 1.42 (d, 3H), 1.18-1.16 (m, 1H), 0.82-0.72 (m, 4H).

### Example 181

### (3R,4S)-3-cyclopropyl-4-methyl-1-(2-(1-methyl-1H-pyrazol-4-yl)-3H-imidazo[4,5-b]pyridin-7-yl)-2-oxopyrrolidine-3-carbonitrile

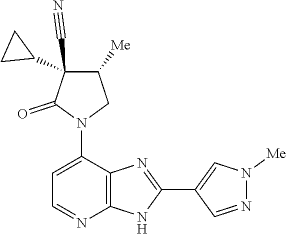 [SMILES: C[C@@H]1CN(c2ccnc3[nH]c(-c4cnn(C)c4)nc23)C(=O)[C@]1(C#N)C1CC1]

To a solution of (3R,4S)-3-cyclopropyl-1-(2,3-diaminopyridin-4-yl)-4-methyl-2-oxopyrrolidine-3-carbonitrile (Preparation 101, 30 mg, 0.110 mmol) in DMF (3.0 mL) was added 1-methyl-1H-pyrazole-4-carbaldehyde (24.4 mg, 0.221 mmol) and TsOH (5.7 mg, 0.033 mmol) and the reaction stirred at 70° C. for 2 h. The cooled mixture was quenched with water (0.5 mL), then purified by prep-HPLC-3 (gradient 17-47%) to give (3R,4S)-3-cyclopropyl-4-methyl-1-(2-(1-methyl-1H-pyrazol-4-yl)-3H-imidazo[4,5-b]pyridin-7-yl)-2-oxopyrrolidine-3-carbonitrile as a yellow solid (12 mg, 30%). LCMS m/z=362.1 [M+H]^{+; 1}H NMR (500 MHz, DMSO-d₆) δ: 13.42 (br s, 1H), 8.39 (s, 1H), 8.20 (d, 1H), 8.12 (s, 1H), 7.80 (d, 1H), 4.82-4.78 (m, 1H), 4.18-4.13 (m, 1H), 3.95 (s, 3H), 3.14-3.09 (m, 1H), 1.45-1.43 (m, 1H), 1.33 (d, 3H), 0.73-0.54 (m, 4H).

### Example 182

### (3R,4S)-3-cyclopropyl-4-methyl-1-(2-(1-methyl-1H-pyrazol-4-yl)imidazo[1,2-b]pyridazin-8-yl)-2-oxopyrrolidine-3-carbonitrile

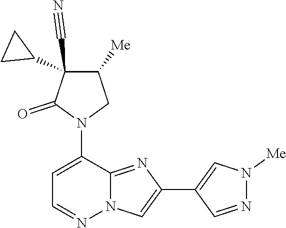 [SMILES: C[C@@H]1CN(c2ccnn3cc(-c4cnn(C)c4)nc23)C(=O)[C@]1(C#N)C1CC1]

To a solution of (3R,4S)-1-(2-bromoimidazo[1,2-b]pyridazin-8-yl)-3-cyclopropyl-4-methyl-2-oxopyrrolidine-3-carbonitrile (Preparation 97, 90 mg, 0.250 mmol), 1-methyl-4-(4,4,5,5-tetramethyl-1,3,2-dioxaborolan-2-yl)-1H-pyrazole (130 mg, 0.625 mmol) in dioxane (2 mL) and H₂O (0.2 mL) was added KF (43.6 mg, 0.750 mmol) and Pd(dppf)Cl₂ (18.3 mg, 0.025 mmol) and the reaction stirred at 70° C. for 6 h under N₂. The cooled mixture was quenched with water (50 mL) and extracted with EtOAc (20 mL×3). The combined organic layers were washed with brine (20 mL), dried (Na₂SO₄) and concentrated in vacuo. The residue was purified by prep-HPLC-3 (gradient 30-60%) to give (3R,4S)-3-cyclopropyl-4-methyl-1-(2-(1-methyl-1H-pyrazol-4-yl)imidazo[1,2-b]pyridazin-8-yl)-2-oxopyrrolidine-3-carbonitrile as a white solid (45 mg, 50%) as a white solid. LCMS m/z=362.1 [M+H]⁺; ¹H NMR (500 MHz, CDCl₃) δ: 8.20 (d, 1H), 8.02 (s, 1H), 7.83 (s, 1H), 7.83-7.79 (m, 2H), 4.91-4.87 (m, 1H), 4.39-4.34 (m, 1H), 3.98 (s, 3H), 3.09-3.01 (m, 1H), 1.44 (d, 3H), 1.21-1.17 (m, 1H), 0.82-0.71 (m, 4H).

### Example 183

### (3R,4S)-3-cyclopropyl-1-(3-fluoro-2-(1-methyl-1H-pyrazol-4-yl)imidazo[1,2-b]pyridazin-8-yl)-4-methyl-2-oxopyrrolidine-3-carbonitrile

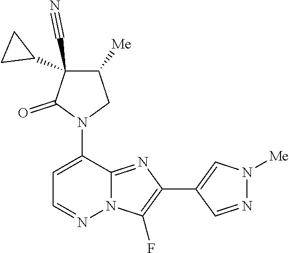 [SMILES: C[C@@H]1CN(c2ccnn3c(F)c(-c4cnn(C)c4)nc23)C(=O)[C@]1(C#N)C1CC1]

(3R,4S)-3-cyclopropyl-1-(3-fluoro-2-(1-methyl-1H-pyrazol-4-yl)imidazo[1,2-b]pyridazin-8-yl)-4-methyl-2-oxopyrrolidine-3-carbonitrile was prepared from (3R,4S)-1-(2-bromo-3-fluoroimidazo[1,2-b]pyridazin-8-yl)-3-cyclopropyl-4-methyl-2-oxopyrrolidine-3-carbonitrile (Preparation 149) and 1-methyl-4-(4,4,5,5-tetramethyl-1,3,2-dioxaborolan-2-yl)-1H-pyrazole using an analogous method to that described for Example 36. HPLC-3 (Gradient: 33-63%); LCMS m/z=380.4 [M+H]⁺; ¹H NMR (500 MHz, CDCl₃) δ: 8.28 (s, 1H), 7.97 (s, 1H), 7.85-7.83 (m, 2H), 4.90-4.89 (m, 1H), 4.35-4.30 (m, 1H), 4.00 (s, 3H), 3.10-3.00 (m, 1H), 1.46-1.45 (m, 3H), 1.19-1.18 (m, 1H), 0.81-0.70 (m, 4H).

### Example 184

### (3R,4S)-3-cyclopropyl-4-methyl-1-(6-(1-methyl-1H-pyrazol-4-yl)pyrazolo[1,5-a]pyrazin-4-yl)-2-oxopyrrolidine-3-carbonitrile

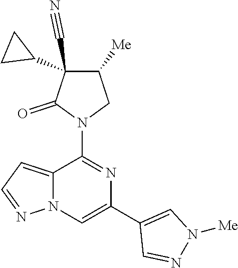 [SMILES: C[C@@H]1CN(c2nc(-c3cnn(C)c3)cn3nccc23)C(=O)[C@]1(C#N)C1CC1]

To a solution of 4-chloro-6-(1-methyl-1H-pyrazol-4-yl)pyrazolo[1,5-a]pyrazine (Preparation 105, 80 mg, 0.342 mmol) and (3R,4S)-3-cyclopropyl-4-methyl-2-oxopyrrolidine-3-carbonitrile (Preparation 21, 84.3 mg, 0.514 mmol) in dioxane (5 mL) was added Pd₂(dba)₃ (31.4 mg, 0.034 mmol), Xantphos (19.8 mg, 0.034 mmol) and K₃PO₄ (101.8 mg, 0.479 mmol) at 25° C. and the reaction stirred at 70° C. for 8 h under N₂. The cooled mixture was concentrated in vacuo and the crude product purified by prep-HPLC-3 (gradient 31 to 61%) to give (3R,4S)-3-cyclopropyl-4-methyl-1-(6-(1-methyl-1H-pyrazol-4-yl)pyrazolo[1,5-a]pyrazin-4-yl)-2-oxopyrrolidine-3-carbonitrile as a white solid (82.9 mg, 67% yield). LCMS m/z=362.2 [M+H]⁺; ¹H NMR (500 MHz, CDCl₃) δ: 8.45 (d, 1H), 7.98 (d, 1H), 7.88 (s, 1H), 7.80 (s, 1H), 7.05 (d, 1H), 4.28-4.24 (m, 1H), 3.98 (s, 3H), 3.96-3.91 (m, 1H), 3.14-3.09 (m, 1H), 1.44 (d, 3H), 1.20-1.19 (m, 1H), 0.83-0.72 (m, 4H).

### Example 185

### (3R,4S)-3-cyclopropyl-1-(6-(1-(difluoromethyl)-1H-pyrazol-4-yl)pyrazolo[1,5-a]pyrazin-4-yl)-4-methyl-2-oxopyrrolidine-3-carbonitrile

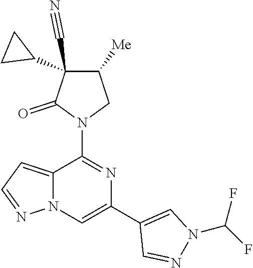 [SMILES: C[C@@H]1CN(c2nc(-c3cnn(C(F)F)c3)cn3nccc23)C(=O)[C@]1(C#N)C1CC1]

To a solution of (3R,4S)-1-(6-chloropyrazolo[1,5-a]pyrazin-4-yl)-3-cyclopropyl-4-methyl-2-oxopyrrolidine-3-carbonitrile (Preparation 110, 50 mg, 0.158 mmol) and 1-(difluoromethyl)-4-(4,4,5,5-tetramethyl-1,3,2-dioxaborolan-2-yl)-1H-pyrazole (58 mg, 0.238 mmol) in dioxane (2 mL) and H₂O (0.2 mL) was added PEPPSI-IPr catalyst (10.8 mg, 0.016 mmol) and K₂CO₃ (43.8 mg, 0.317 mmol) and the reaction stirred at 90° C. for 3 h under N₂. The cooled mixture was quenched with water (30 mL) and extracted with EtOAc (3×20 mL). The combined organics were washed with brine (20 mL), dried over Na₂SO₄, filtered and concentrated in vacuo. The crude product was purified by prep-HPLC-3 (gradient 39-59%) to give (3R,4S)-3-cyclopropyl-1-(6-(1-(difluoromethyl)-1H-pyrazol-4-yl)pyrazolo[1,5-a]pyrazin-4-yl)-4-methyl-2-oxopyrrolidine-3-carbonitrile as a white solid (28 mg, 44% yield). LCMS m/z=398.1 [M+H]⁺; ¹H NMR (500 MHz, CDCl₃) δ: 8.54 (s, 1H), 8.26 (s, 1H), 8.05-8.03 (m, 2H), 7.25 (t, 1H), 7.12 (s, 1H), 4.29-4.25 (m, 1H), 3.99-3.94 (m, 1H), 3.17-3.10 (m, 1H), 1.46 (d, 3H), 1.22-1.20 (m, 1H), 0.84-0.73 (m, 4H).

### Example 186

### (3R,4S)-3-cyclopropyl-4-methyl-1-(3-methyl-6-(1-methyl-1H-pyrazol-4-yl)pyrazolo[1,5-a]pyrazin-4-yl)-2-oxopyrrolidine-3-carbonitrile

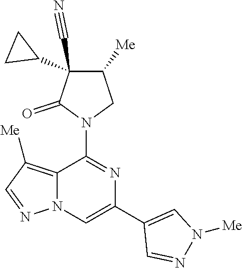 [SMILES: Cc1cnn2cc(-c3cnn(C)c3)nc(N3C[C@@H](C)[C@@](C#N)(C4CC4)C3=O)c12]

(3R,4S)-3-Cyclopropyl-4-methyl-1-(3-methyl-6-(1-methyl-1H-pyrazol-4-yl)pyrazolo[1,5-a]pyrazin-4-yl)-2-oxopyrrolidine-3-carbonitrile was obtained as a white solid, 71.4 mg, 52.3% yield, from (3R,4S)-1-(6-chloro-3-methylpyrazolo[1,5-a]pyrazin-4-yl)-3-cyclopropyl-4-methyl-2-oxopyrrolidine-3-carbonitrile (Preparation 111) and 1-methyl-4-(4,4,5,5-tetramethyl-1,3,2-dioxaborolan-2-yl)-1H-pyrazole, following a similar procedure to that described in Example 38. LCMS m/z=376.1 [M+H]⁺; ¹H NMR (500 MHz, CDCl₃) δ: 8.38 (s, 1H), 7.85 (s, 1H), 7.78 (s, 2H), 4.10-4.09 (m, 1H), 3.96 (s, 3H), 3.93-3.89 (m, 1H), 3.19-3.15 (m, 1H), 2.36 (s, 3H), 1.44 (d, 3H), 1.22-1.21 (m, 1H), 0.89-0.76 (m, 4H).

### Example 187 to 205

The compounds in the following table were prepared from the appropriate halide and boronate ester, following a similar procedure to that described for Example 186,

| | |
|---|---|
| Example No | Name/Structure/Heterocycle/Yield/Data |

(3R,4S)-1-(6-chloro-3-fluoropyrazolo[1,5-a]pyrazin-4-yl)-3-cyclopropyl-4-methyl-2-
oxopyrrolidine-3-carbonitrile (Preparation 125)

| | |
|---|---|
| 187 | (3R,4S)-3-cyclopropyl-1-(3-fluoro-6-(1-methyl-1H-pyrazol-4- |
| | yl)pyrazolo[1,5-a]pyrazin-4-yl)-4-methyl-2-oxopyrrolidine-3- |
| | carbonitrile |
| | 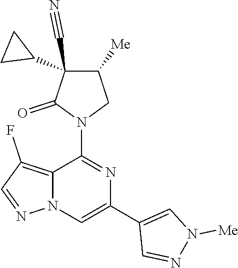 [SMILES: C[C@@H]1CN(c2nc(-c3cnn(C)c3)cn3ncc(F)c23)C(=O)[C@]1(C#N)C1CC1] |
| | Het: 1-methyl-4-(4,4,5,5-tetramethyl-1,3,2-dioxaborolan-2-yl)-1H-pyrazole |
| | HPLC-3 (gradient: 30-60%); White solid (36 mg, 48%). |
| | LCMS m/z = 380.1 [M + H]⁺; ¹H NMR (400 MHz, CDCl₃) δ: 8.24 (s, |
| | 1H), 7.86 (s, 1H), 7.82-7.80 (m, 2H), 4.31-4.26 (m, 1H), 3.97 (s, 3H), |
| | 3.81 (t, 1H), 3.22-3.14 (m, 1H), 1.44 (d, 3H), 1.25-1.19 (m, 1H), 0.86- |
| | 0.75 (m, 4H). |
| 188 | (3R,4S)-3-cyclopropyl-1-(6-(1-ethyl-1H-pyrazol-4-yl)-3- |
| | fluoropyrazolo[1,5-a]pyrazin-4-yl)-4-methyl-2-oxopyrrolidine-3- |
| | carbonitrile |
| | 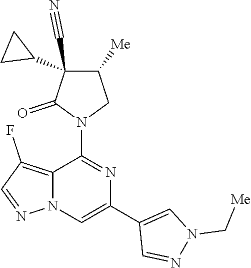 [SMILES: CCn1cc(-c2cn3ncc(F)c3c(N3C[C@@H](C)[C@@](C#N)(C4CC4)C3=O)n2)cn1] |
| | Het: 1-ethyl-4-(4,4,5,5-tetramethyl-1,3,2-dioxaborolan-2-yl)-1H-pyrazole |
| | HPLC-3 (gradient: 35-65%); White solid (18.5 mg, 52%). |
| | LCMS m/z = 394.1 [M + H]⁺; ¹H NMR: (400 MHz, CDCl₃) δ: 8.24 (s, |
| | 1H), 7.87-7.80 (m, 3H), 4.32-4.22 (m, 3H), 3.83-3.78 (m, 1H), 3.18- |
| | 3.15 (m, 1H), 1.58-1.53 (m, 3H), 1.45-1.43 (m, 3H), 1.23-1.21 (m, 1H), |
| | 0.85-0.75 (m, 4H). |
| 189 | (3R,4S)-3-cyclopropyl-1-(6-(1-(difluoromethyl)-1H-pyrazol-4-yl)-3- |
| | fluoropyrazolo[1,5-a]pyrazin-4-yl)-4-methyl-2-oxopyrrolidine-3- |
| | carbonitrile |
| | 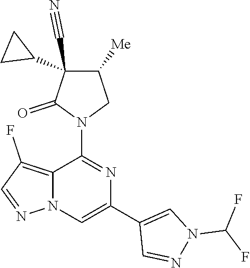 [SMILES: C[C@@H]1CN(c2nc(-c3cnn(C(F)F)c3)cn3ncc(F)c23)C(=O)[C@]1(C#N)C1CC1] |
| | Het: 1-(difluoromethyl)-4-(4,4,5,5-tetramethyl-1,3,2-dioxaborolan-2-yl)-1H- |
| | pyrazole |
| | HPLC-3 (gradient 38-68%); White solid (53 mg, 53%). |
| | LCMS m/z = 416.1 [M + H]⁺; ¹H NMR: (400 MHz, CDCl₃) δ: 8.31 (s, |
| | 1H), 8.25 (s, 1H), 8.01 (s, 1H), 7.85 (d, 1H), 7.22 (t, 1H), 4.30-4.25 (m, |
| | 1H), 3.82 (t, 1H), 3.21-3.13 (m, 1H), 1.43 (d, 3H), 1.24-1.19 (m, 1H), |
| | 0.85-0.81 (m, 2 H), 0.79-0.75 (m, 2H). |
| 190 | (3R,4S)-3-cyclopropyl-1-(3-fluoro-6-(1-isopropyl-1H-pyrazol-4- |
| | yl)pyrazolo[1,5-a]pyrazin-4-yl)-4-methyl-2-oxopyrrolidine-3- |
| | carbonitrile |
| | 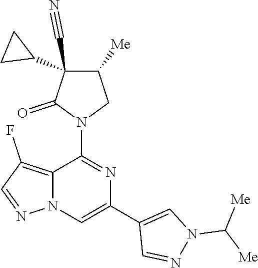 [SMILES: CC(C)n1cc(-c2cn3ncc(F)c3c(N3C[C@@H](C)[C@@](C#N)(C4CC4)C3=O)n2)cn1] |
| | Het: 1-(dimethyl)-4-(4,4,5,5-tetramethyl-1,3,2-dioxaborolan-2-yl)-1H- |
| | pyrazole |
| | HPLC-3 (gradient: 23-53%); White solid (37 mg, 33%). |
| | LCMS m/z = 408.2 [M + H]⁺; ¹H NMR (500 MHz, CDCl₃) δ: 8.25 (s, |
| | 1H), 7.87-7.85 (m, 2H), 7.81 (d, 1H), 4.59-4.54 (m, 1H), 4.33-4.28 (m, |
| | 1H), 3.83-3.78 (m, 1H), 3.20-3.13 (m, 1H), 1.57 (d, 6H), 1.44 (d, 3H), |
| | 1.22-1.20 (m, 1H), 0.87-0.75 (m, 4H). |
| 191 | (3R,4S)-3-cyclopropyl-1-(3-fluoro-6-(1-((S)-tetrahydrofuran-3-yl)-1H- |
| | pyrazol-4-yl)pyrazolo[1,5-a]pyrazin-4-yl)-4-methyl-2-oxopyrrolidine-3- |
| | carbonitrile |
| | 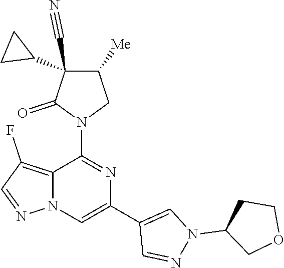 [SMILES: C[C@@H]1CN(c2nc(-c3cnn([C@H]4CCOC4)c3)cn3ncc(F)c23)C(=O)[C@]1(C#N)C1CC1] |
| | Het: (S)-1-(tetrahydrofuran-3-yl)-4-(4,4,5,5-tetramethyl-1,3,2- |
| | dioxaborolan-2-yl)-1H-pyrazole |
| | HPLC-3 (gradient: 33-63%); White solid (38.2 mg, 33%). |
| | LCMS m/z = 436.1 [M + H]⁺; ¹H NMR (500 MHz, CDCl₃) δ: 8.26 (s, |
| | 1H), 7.93 (s, 1H), 7.88 (s, 1H), 7.83 (d, 1H), 5.07-5.05 (m, 1H), 4.32- |
| | 4.29 (m, 1H), 4.20-4.16 (m, 2H), 4.11-4.09 (m, 1H), 4.00-3.97 (m, 1H), |
| | 3.82-3.81 (m, 1H), 3.20-3.17 (m, 1H), 2.54-2.52 (m, 1H), 2.39-2.37 (m, |
| | 1H), 1.46 (d, 3H), 1.22-1.21 (m, 1H), 0.88-0.77 (m, 4H). |
| 192 | (3R,4S)-3-cyclopropyl-1-(6-(6,7-dihydro-4H-pyrazolo[5,1- |
| | c][1,4]oxazin-3-yl)-3-fluoropyrazolo[1,5-a]pyrazin-4-yl)-4-methyl-2- |
| | oxopyrrolidine-3-carbonitrile |
| | 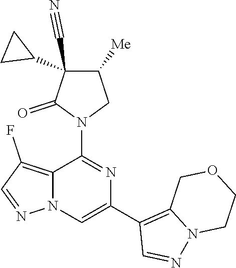 [SMILES: C[C@@H]1CN(c2nc(-c3cnn4c3COCC4)cn3ncc(F)c23)C(=O)[C@]1(C#N)C1CC1] |
| | Het: 3-(4,4,5,5-tetramethyl-1,3,2-dioxaborolan-2-yl)-6,7-dihydro-4H- |
| | pyrazolo[5,1-c][1,4]oxazine (Preparation 112) |
| | HPLC-3 (gradient: 32-62%); White solid (15.5 mg, 41%). |
| | LCMS m/z = 422.2 [M + H]⁺; ¹H NMR: (500 MHz, DMSO-d₆) δ: 9.01 |
| | (s, 1H), 8.23 (d, 1H), 8.16 (s, 1H), 5.14 (s, 2H), 4.23-4.22 (m, 1H), |
| | 4.18-4.07 (m, 4H), 3.88-3.84 (m, 1H), 3.19-3.14 (m, 1H), 1.50-1.48 (m, |
| | 1H), 1.33 (d, 3H), 0.54-0.74 (m, 4H). |

Using (3R,4S)-1-(6-chloropyrazolo[1,5-a]pyrazin-4-yl)-3-cyclopropyl-4-methyl-2-
oxopyrrolidine-3-carbonitrile (Preparation 110)

| | |
|---|---|
| 193* | (3R,4S)-3-cyclopropyl-1-(6-(6-methoxypyridin-2-yl)pyrazolo[1,5- |
| | a]pyrazin-4-yl)-4-methyl-2-oxopyrrolidine-3-carbonitrile |
| | 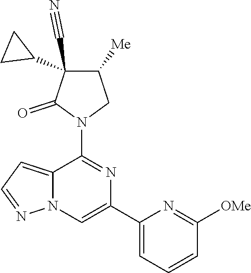 [SMILES: COc1cccc(-c2cn3nccc3c(N3C[C@@H](C)[C@@](C#N)(C4CC4)C3=O)n2)n1] |
| | Het: 2-methoxy-6-(4,4,5,5-tetramethyl-1,3,2-dioxaborolan-2-yl)pyridine |
| | HPLC-3 (gradient: 58-78%); White solid (34.2 mg, 40%). |
| | LCMS m/z = 389.2 [M + H]⁺; ¹H NMR: (500 MHz, CDCl₃) δ: 9.35 (s, |
| | 1H), 8.07 (d, 1H), 7.77 (d, 1H), 7.79-7.66 (m, 1H), 7.14 (s, 1H), 6.75 (d, |
| | 1H), 4.35-4.31 (m, 1H), 4.04-3.99 (m, 4H), 3.17-3.11 (m, 1H), 1.46 (d, |
| | 3H), 1.22-1.20 (m, 1H), 0.86-0.73 (m, 4H). |
| 194 | (3R,4S)-3-cyclopropyl-4-methyl-2-oxo-1-(6-(pyridin-3-yl)pyrazolo[1,5- |
| | a]pyrazin-4-yl)pyrrolidine-3-carbonitrile |
| | 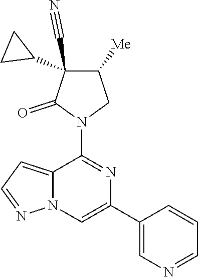 [SMILES: C[C@@H]1CN(c2nc(-c3cccnc3)cn3nccc23)C(=O)[C@]1(C#N)C1CC1] |
| | Het: (pyridin-3-yl)boronic acid |
| | HPLC-3 (gradient: 35-65%); White solid (35.1 mg, 51%). |
| | LCMS m/z = 359.1 [M + H]⁺; ¹H NMR (400 MHz, CDCl₃) δ: 9.22 (s, |
| | 1H), 8.78 (s, 1H), 8.68 (s, 1H), 8.22 (d, 1H), 8.09 (d, 1H), 7.47-7.43 (m, |
| | 1H), 7.20 (d, 1H), 4.36-4.30 (m, 1H), 4.03 (t, 1H), 3.18-3.11 (m, 1H), |
| | 1.47 (d, 3H), 1.22-1.20 (m, 1H), 0.87-0.74 (m, 4H). |
| 195 | 5-(4-((3R,4S)-3-cyano-3-cyclopropyl-4-methyl-2-oxopyrrolidin-1- |
| | yl)pyrazolo[1,5-a]pyrazin-6-yl)picolinonitrile |
| | 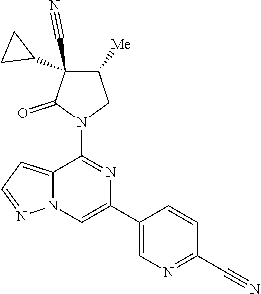 [SMILES: C[C@@H]1CN(c2nc(-c3ccc(C#N)nc3)cn3nccc23)C(=O)[C@]1(C#N)C1CC1] |
| | Het: (6-cyanopyridin-3-yl)boronic acid |
| | HPLC-3 (gradient: 42-72%); White solid (40.6 mg, 56%). |
| | LCMS m/z = 384.1 [M + H]⁺; ¹H NMR (400 MHz, CDCl₃) δ: 9.30 (d, |
| | 1H), 8.85 (s, 1H), 8.36-8.33 (m, 1H), 8.12 (d, 1H), 7.81 (d, 1H), 7.25 (d, |
| | 1H), 4.34-4.28 (m, 1H), 4.03-3.97 (m, 1H), 3.19-3.11 (m, 1H), 1.47 (d, |
| | 3H), 1.25-1.20 (m, 1H), 0.86-0.74 (m, 4H). |
| 196 | (3R,4S)-3-cyclopropyl-1-(6-(5-fluoro-2-methoxypyridin-4- |
| | yl)pyrazolo[1,5-a]pyrazin-4-yl)-4-methyl-2-oxopyrrolidine-3- |
| | carbonitrile |
| | 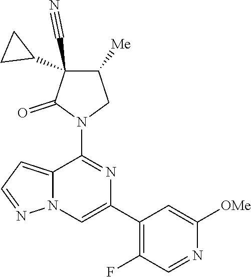 [SMILES: COc1cc(-c2cn3nccc3c(N3C[C@@H](C)[C@@](C#N)(C4CC4)C3=O)n2)c(F)cn1] |
| | Het: (5-fluoro-2-methoxypyridin-4-yl)boronic acid |
| | HPLC-3 (gradient: 49-69%); White solid (21 mg, 16%). |
| | LCMS m/z = 407.1 [M + H]⁺; ¹H NMR: (500 MHz, DMSO-d₆) δ: 9.17 |
| | (s, 1H), 8.35-8.31 (m, 2H), 7.53 (d, 1H), 7.12 (s, 1H), 4.38-4.34 (m, |
| | 1H), 4.04-3.99 (m, 1H), 3.92 (s, 3H), 3.21-3.17 (m, 1H), 1.56-1.52 (m, |
| | 1H), 1.34 (d, 3H), 0.78-0.57 (m, 4H). |
| 197* | 4-(4-((3R,4S)-3-cyano-3-cyclopropyl-4-methyl-2-oxopyrrolidin-1- |
| | yl)pyrazolo[1,5-a]pyrazin-6-yl)picolinonitrile |
| | 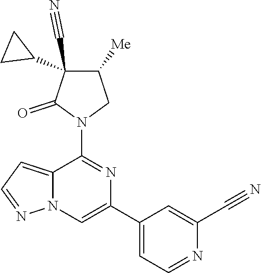 [SMILES: C[C@@H]1CN(c2nc(-c3ccnc(C#N)c3)cn3nccc23)C(=O)[C@]1(C#N)C1CC1] |
| | Het: 4-(4,4,5,5-tetramethyl-1,3,2-dioxaborolan-2-yl)picolinonitrile |
| | HPLC-3 (gradient: 39-69%): White solid (25.2 mg, 21%). |
| | LCMS m/z = 384.1 [M + H]⁺; ¹H NMR (400 MHz, CDCl₃) δ: 8.91 (s, |
| | 1H), 8.82 (d, 1H), 8.32 (s, 1H), 8.15 (s, 1H), 7.99-7.97 (m, 1H), 7.28 (s, |
| | 1H), 4.35-4.30 (m, 1H), 4.08-4.02 (m, 1H), 3.20-3.14 (m, 1H), 1.51 (d, |
| | 3H), 1.23-1.21 (m, 1H), 0.85-0.76 (m, 4H). |
| 198* | (3R,4S)-3-cyclopropyl-1-(6-(1-(2,2-difluorocyclopropyl)-1H-pyrazol-4- |
| | yl)pyrazolo[1,5-a]pyrazin-4-yl)-4-methyl-2-oxopyrrolidine-3- |
| | 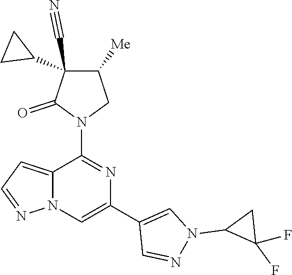 [SMILES: C[C@@H]1CN(c2nc(-c3cnn(C4CC4(F)F)c3)cn3nccc23)C(=O)[C@]1(C#N)C1CC1] |
| | carbonitrile F |
| | Het: 1-(2,2-difluorocyclopropyl)-4-(4,4,5,5-tetramethyl-1,3,2- |
| | dioxaborolan-2-yl)-1H-pyrazole (Preparation 115) |
| | HPLC-3 (gradient: 27-67%); White solid (12.1 mg, 30%). |
| | LCMS m/z = 424.1 [M + H]⁺; ¹H NMR (400 MHz, CDCl₃) δ: 8.49 (s, |
| | 1H), 8.01 (d, 1H), 7.97-7.93 (m, 2H), 7.09 (d, 1H), 4.29-4.24 (m, 1H), |
| | 4.17-4.15 (m, 1H), 3.98-3.94 (m, 1H), 3.14-3.11 (m, 1H), 2.33-2.29 (m, |
| | 1H), 2.18-2.15 (m, 1H), 1.46 (d, 3H), 1.22-1.19 (m, 1H), 0.84-0.73 (m, |
| | 4H). |
| 199 | (3R,4S)-3-cyclopropyl-1-(6-(1,5-dimethyl-1H-pyrazol-4- |
| | yl)pyrazolo[1,5-a]pyrazin-4-yl)-4-methyl-2-oxopyrrolidine-3- |
| | carbonitrile |
| | 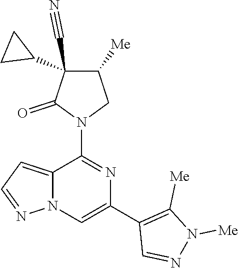 [SMILES: Cc1c(-c2cn3nccc3c(N3C[C@@H](C)[C@@](C#N)(C4CC4)C3=O)n2)cnn1C] |
| | Het: 1,5-dimethyl-4-(4,4,5,5-tetramethyl-1,3,2-dioxaborolan-2-yl)-1H- |
| | pyrazole |
| | HPLC-3 (gradient: 32-62%); White solid (56.1 mg, 78%). |
| | LCMS m/z = 376.1 [M + H]⁺; ¹H NMR (400 MHz, CDCl₃) δ: 8.40 (s, |
| | 1H), 8.01 (d, 1H), 7.76 (s, 1H), 7.06 (d, 1H), 4.26-4.21 (m, 1H), 3.95- |
| | 3.89 (m, 1H), 3.87 (s, 3H), 3.15-3.07 (m, 1H), 2.55 (s, 3H), 1.44 (d, |
| | 3H), 1.22-1.19 (m, 1H), 0.84-0.72 (m, 4H). |
| 200 | (3R,4S)-3-cyclopropyl-4-methyl-2-oxo-1-(6-(1,3,5-trimethyl-1H- |
| | pyrazol-4-yl)pyrazolo[1,5-a]pyrazin-4-yl)pyrrolidine-3-carbonitrile |
| | 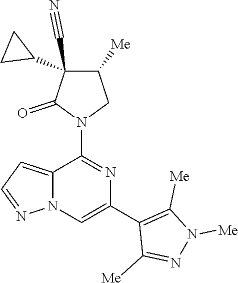 [SMILES: Cc1nn(C)c(C)c1-c1cn2nccc2c(N2C[C@@H](C)[C@@](C#N)(C3CC3)C2=O)n1] |
| | Het: 1,3,5-trimethyl-4-(4,4,5,5-tetramethyl-1,3,2-dioxaborolan-2-yl)- |
| | 1H-pyrazole |
| | HPLC-3 (gradient: 32-62%); White solid (56.1 mg, 78%). |
| | LCMS m/z = 390.1 [M + H]⁺; ¹H NMR (400 MHz, MeOH-d₄) δ: 8.45 (s, |
| | 1H), 8.05 (d, 1H), 7.00 (d, 1H), 4.30-4.25 (m, 1H), 3.91 (t, 1H), 3.77 (s, |
| | 3 H) 3.19-3.11 (m, 1H), 2.42 (s, 3H), 2.34 (s, 3H), 1.39 (d, 3H) 1.36- |
| | 1.34 (m, 1H), 0.78-0.68 (m, 4H). |
| 201 | (3R,4S)-3-cyclopropyl-4-methyl-1-(6-(1-(methyl-d3)-1H-pyrazol-4- |
| | yl)pyrazolo[1,5-a]pyrazin-4-yl)-2-oxopyrrolidine-3-carbonitrile |
| | 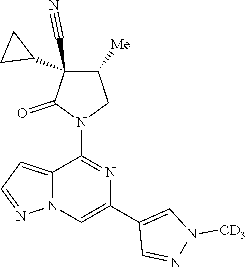 [SMILES: [2H]C([2H])([2H])n1cc(-c2cn3nccc3c(N3C[C@@H](C)[C@@](C#N)(C4CC4)C3=O)n2)cn1] |
| | Het: 1-(methyl-d3)-4-(4,4,5,5-tetramethyl-1,3,2-dioxaborolan-2-yl)-1H- |
| | pyrazole (Preparation 117) |
| | HPLC-3 (gradient: 29-59%); White solid (42.1 mg, 61%). |
| | LCMS m/z = 365.2 [M + H]⁺; ¹H NMR (500 MHz, CDCl₃) δ: 8.46 (s, |
| | 1H), 7.99 (d, 1H), 7.89 (s, 1H), 7.80 (s, 1H), 7.06 (d, 1H), 4.29-4.25 (m, |
| | 1H), 3.97-3.92 (m, 1H), 3.16-3.09 (m, 1H), 1.45 (d, 3H), 1.20-1.19 (m, |
| | 1H), 0.84-0.73 (m, 4H). |
| 202* | (3R,4S)-3-cyclopropyl-1-(6-(1-ethyl-1H-pyrazol-4-yl)pyrazolo[1,5- |
| | a]pyrazin-4-yl)-4-methyl-2-oxopyrrolidine-3-carbonitrile |
| | 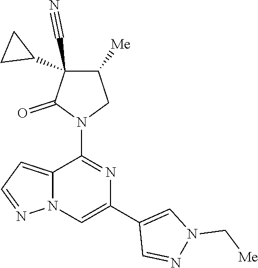 [SMILES: CCn1cc(-c2cn3nccc3c(N3C[C@@H](C)[C@@](C#N)(C4CC4)C3=O)n2)cn1] |
| | Het: 1-ethyl-4-(4,4,5,5-tetramethyl-1,3,2-dioxaborolan-2-yl)-1H- |
| | pyrazole |
| | Prep-TLC; White solid (60 mg, 34%). |
| | LCMS m/z = 376.1 [M + H]⁺; ¹H NMR (500 MHz, CDCl₃) δ: 8.43 (s, |
| | 1H), 7.99 (d, 1H), 7.89 (s, 1H), 7.83 (s, 1H), 7.06 (d, 1H), 4.30-4.26 (m, |
| | 1H), 4.25-4.22 (m, 2H), 3.97-3.92 (m, 1H), 3.13-3.09 (m, 1H), 1.55 (d, |
| | 3H), 1.45 (d, 3H), 1.21-1.19 (m, 1H), 0.85-0.72 (m, 4H). |
| 203 | (3R,4S)-3-cyclopropyl-4-methyl-2-oxo-1-(6-(1-(tetrahydro-2H-pyran-4- |
| | yl)-1H-pyrazol-4-yl)pyrazolo[1,5-a]pyrazin-4-yl)pyrrolidine-3- |
| | carbonitrile |
| | 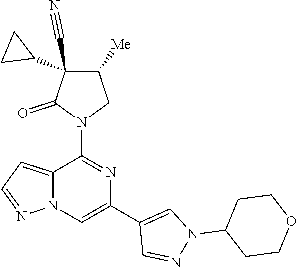 [SMILES: C[C@@H]1CN(c2nc(-c3cnn(C4CCOCC4)c3)cn3nccc23)C(=O)[C@]1(C#N)C1CC1] |
| | Het: 1-(tetrahydro-2H-pyran-4-yl)-4-(4,4,5,5-tetramethyl-1,3,2- |
| | dioxaborolan-2-yl)-1H-pyrazole |
| | HPLC-3 (gradient: 32-62%); White solid (68 mg, 33%). |
| | LCMS m/z = 432.2 [M + H]⁺; ¹H NMR (500 MHz, CDCl₃) δ: 8.47 (s, |
| | 1H), 8.00 (d, 1H), 7.91 (s, 1H), 7.88 (s, 1H), 7.05 (d, 1H), 4.42-4.27 (m, |
| | 1H), 4.26-4.23 (m, 1H), 4.16-4.13 (m, 2H), 3.97-3.92 (m, 1H), 3.58- |
| | 3.56 (m, 2H), 3.14-3.11 (m, 1H), 2.18-2.11 (m, 4H), 1.45 (d, 3H), 1.21- |
| | 1.19 (m, 1H), 0.85-0.72 (m, 4H). |
| 204 | (3R,4S)-3-cyclopropyl-4-methyl-2-oxo-1-(6-(7-oxo-5,6,7,8-tetrahydro- |
| | 1,8-naphthyridin-3-yl)pyrazolo[1,5-a]pyrazin-4-yl)pyrrolidine-3- |
| | carbonitrile |
| | 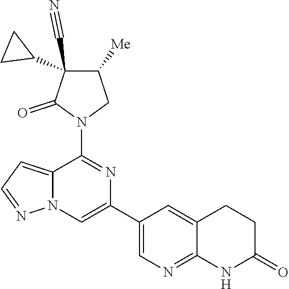 [SMILES: C[C@@H]1CN(c2nc(-c3cnc4c(c3)CCC(=O)N4)cn3nccc23)C(=O)[C@]1(C#N)C1CC1] |
| | Het: 6-(4,4,5,5-tetramethyl-1,3,2-dioxaborolan-2-yl)-3,4-dihydro-1,8- |
| | naphthyridin-2(1H)-one (Preparation 113) |
| | HPLC-3 (gradient: 30-60%); White solid (45.8 mg, 52%). |
| | LCMS m/z = 428.3 [M + H]⁺; ¹H NMR (500 MHz, DMSO-d₆) δ: 10.65 |
| | (s, 1H), 9.39 (s, 1H), 8.87 (d, 1H), 8.32 (d, 1H), 8.21 (s, 1H), 7.00 (d, |
| | 1H), 4.42-4.38 (m, 1H), 4.02-3.97 (m, 1H), 3.22-3.19 (m, 1H), 3.01- |
| | 2.98 (m, 2H), 2.58 (d, 2H), 1.53-1.52 (m, 1H), 1.34 (d, 3H), 0.76-0.71 |
| | (m, 1H), 0.70-0.69 (m, 1H), 0.60-0.57 (m, 2H). |
| 205 | (3R,4S)-3-cyclopropyl-4-methyl-2-oxo-1-(6-(2-oxo-2,3-dihydro-1H- |
| | pyrrolo[2,3-b]pyridin-5-yl)pyrazolo[1,5-a]pyrazin-4-yl)pyrrolidine-3- |
| | carbonitrile |
| | 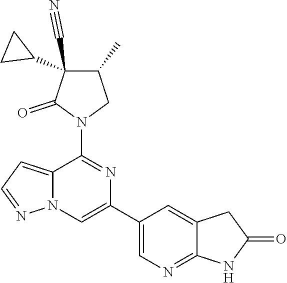 [SMILES: C[C@@H]1CN(c2nc(-c3cnc4c(c3)CC(=O)N4)cn3nccc23)C(=O)[C@]1(C#N)C1CC1] |
| | Het: 5-(4,4,5,5-tetramethyl-1,3,2-dioxaborolan-2-yl)-1,3-dihydro-2H- |
| | pyrrolo[2,3-b]pyridin-2-one (Preparation 114) |
| | HPLC-3 (gradient: 31-61%); White solid (18.3 mg, 14%). |
| | LCMS m/z = 414.2 [M + H]⁺; ¹H NMR (400 MHz, DMSO-d₆) δ: 11.16 |
| | (s, 1H), 9.38 (s, 1H), 8.84 (d, 1H), 8.27 (s, 1H), 8.19 (d, 1H), 6.99 (d, |
| | 1H), 4.40-4.35 (m, 1H), 4.00 (t, 1H), 3.66 (s, 2H), 3.25-3.15 (m, 1H), |
| | 1.57-1.48 (m, 1H), 1.34 (d, 3H), 0.78-0.55 (m, 4H). |

### Example 206

### (3R,4S)-1-(3-chloro-6-(1-methyl-1H-pyrazol-4-yl)pyrazolo[1,5-a]pyrazin-4-yl)-3-cyclopropyl-4-methyl-2-oxopyrrolidine-3-carbonitrile

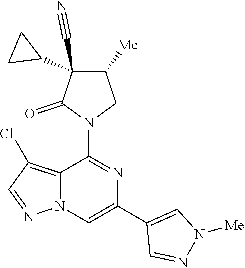 [SMILES: C[C@@H]1CN(c2nc(-c3cnn(C)c3)cn3ncc(Cl)c23)C(=O)[C@]1(C#N)C1CC1]

To a solution of (3R,4S)-3-cyclopropyl-4-methyl-2-oxopyrrolidine-3-carbonitrile (Preparation 21, 58.8 mg, 0.358 mmol) in DMF (3 mL) was added NaH (14.3 mg, 0.358 mmol, 60% purity). After stirring for 10 min, 3,4-dichloro-6-(1-methyl-1H-pyrazol-4-yl)pyrazolo[1,5-a]pyrazine (Preparation 123, 80 mg, 0.298 mmol) was added and the mixture was stirred at 20° C. for 30 min. The reaction was quenched with water (0.5 mL) and purified by prep-HPLC-3 (gradient: 33-63%) to afford (3R,4S)-1-(3-chloro-6-(1-methyl-1H-pyrazol-4-yl)pyrazolo[1,5-a]pyrazin-4-yl)-3-cyclopropyl-4-methyl-2-oxopyrrolidine-3-carbonitrile as a yellow solid (56 mg, 47%). LCMS m/z=396.1 [M+H]⁺; ¹H NMR (500 MHz, CDCl₃) δ: 8.38 (s, 1H), 7.93 (s, 1H), 7.86 (s, 1H), 7.82 (s, 1H), 4.18-4.14 (m, 1H), 3.97 (s, 3H), 3.86-3.82 (m, 1H), 3.27-3.19 (m, 1H), 1.45 (d, 3H), 1.25-1.23 (m, 1H), 0.91-0.75 (m, 4H).

### Example 207

### (3R,4S)-1-(6-(1-(bicyclo[1.1.1]pentan-1-yl)-1H-pyrazol-4-yl)-3-fluoropyrazolo[1,5-a]pyrazin-4-yl)-3-cyclopropyl-4-methyl-2-oxopyrrolidine-3-carbonitrile

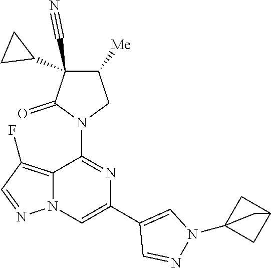 [SMILES: C[C@@H]1CN(c2nc(-c3cnn(C45CC(C4)C5)c3)cn3ncc(F)c23)C(=O)[C@]1(C#N)C1CC1]

To a solution of (3R,4S)-3-cyclopropyl-1-(3-fluoro-6-(tributylstannyl)pyrazolo[1,5-a]pyrazin-4-yl)-4-methyl-2-oxopyrrolidine-3-carbonitrile (Preparation 126, 65 mg, 0.110 mmol) in DMF (3 mL) was added 1-(bicyclo[1.1.1]pentan-1-yl)-4-iodo-1H-pyrazole (Preparation 121, 31.6 mg, 0.122 mmol), CuI (2.10 mg, 0.011 mmol) and Pd(PPh₃)₄ (12.8 mg, 0.011 mmol) and the reaction stirred at 100° C. for 2 h. The mixture was concentrated under vacuum and the residue purified by prep-HPLC-2 (gradient: 50-80%) to give (3R,4S)-1-(6-(1-(bicyclo[1.1.1]pentan-1-yl)-1H-pyrazol-4-yl)-3-fluoropyrazolo[1,5-a]pyrazin-4-yl)-3-cyclopropyl-4-methyl-2-oxopyrrolidine-3-carbonitrile as a yellow solid (16 mg, 34%). LCMS m/z=432.2 [M+H]⁺ 1H NMR: (500 MHz, CDCl₃) δ: 8.25 (s, 1H), 7.88 (s, 1H), 7.83 (s, 1H), 7.82-7.80 (m, 1H), 4.33-4.28 (m, 1H), 3.83-3.78 (m, 1H), 3.20-3.15 (m, 1H), 2.66 (s, 1H), 2.36 (s, 6H), 1.44 (d, 3H), 1.21-1.19 (m, 1H), 0.88-0.77 (m, 4H).

### Example 208

### (3R,4S)-3-cyclopropyl-1-(6-(1,3-dimethyl-1H-pyrazol-4-yl)pyrazolo[1,5-a]pyrazin-4-yl)-4-methyl-2-oxopyrrolidine-3-carbonitrile

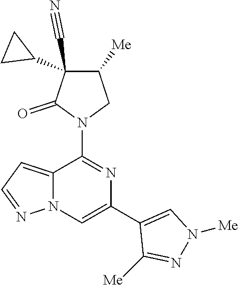 [SMILES: Cc1nn(C)cc1-c1cn2nccc2c(N2C[C@@H](C)[C@@](C#N)(C3CC3)C2=O)n1]

To a solution of (3R,4S)-1-(6-chloropyrazolo[1,5-a]pyrazin-4-yl)-3-cyclopropyl-4-methyl-2-oxopyrrolidine-3-carbonitrile (Preparation 110, 100 mg, 0.317 mmol) and 1,3-dimethyl-4-(4,4,5,5-tetramethyl-1,3,2-dioxaborolan-2-yl)-1H-pyrazole (141 mg, 0.633 mmol) in dioxane (5 mL) and H₂O (1 mL) was added BrettPhos Pd G3 (28.7 mg, 0.032 mmol) and K₂CO₃ (131 mg, 0.950 mmol) and the reaction stirred at 100° C. for 2 h under N₂. The mixture was concentrated under reduced pressure and the residue was purified by prep-HPLC-3 (gradient: 33-63%) to give (3R,4S)-3-cyclopropyl-1-(6-(1,3-dimethyl-1H-pyrazol-4-yl)pyrazolo[1,5-a]pyrazin-4-yl)-4-methyl-2-oxopyrrolidine-3-carbonitrile as a white solid (70.6 mg, 59%). LCMS m/z=376.1 [M+H]⁺; ¹H NMR (400 MHz, CDCl₃) δ: 8.41 (s, 1H), 8.00 (d, 1H), 7.73 (s, 1H), 7.06 (d, 1H), 4.26-4.21 (m, 1H), 3.95-3.90 (m, 4H), 3.15-3.08 (m, 1H), 2.52 (s, 1H), 1.44 (d, 3H), 1.21-1.18 (m, 1H), 0.84-0.76 (m, 4H).

### Example 209

### (3R,4S)-3-cyclopropyl-1-(6-(2,4-dimethylthiazol-5-yl)pyrazolo[1,5-a]pyrazin-4-yl)-4-methyl-2-oxopyrrolidine-3-carbonitrile

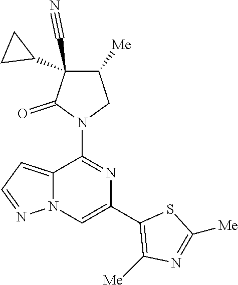 [SMILES: Cc1nc(C)c(-c2cn3nccc3c(N3C[C@@H](C)[C@@](C#N)(C4CC4)C3=O)n2)s1]

The title compound was prepared as a white solid (17.9 mg, 36%) from (3R,4S)-1-(6-chloropyrazolo[1,5-a]pyrazin-4-yl)-3-cyclopropyl-4-methyl-2-oxopyrrolidine-3-carbonitrile (Preparation 110) and 2,4-dimethyl-5-(4,4,5,5-tetramethyl-1,3,2-dioxaborolan-2-yl)thiazole using an analogous method to that described for Example 208. Prep-HPLC-3 (Gradient: 40-70%); LCMS m/z=393.1 [M+H]⁺; ¹H NMR (500 MHz, DMSO-d₆) δ: 8.91 (s, 1H), 8.22 (d, 1H), 7.04 (d, 1H), 4.29-4.24 (m, 1H), 3.87 (t, 1H), 3.23-3.15 (m, 1H), 2.65 (s, 3H), 2.59 (s, 3H), 1.62-1.42 (m, 1H), 1.32 (d, 3H), 0.78-0.71 (m, 1H), 0.70-0.65 (m, 1H), 0.62-0.52 (m, 2H).

### Example 210

### (3R,4S)-3-cyclopropyl-1-(6-(1-(difluoromethyl)-6-oxo-1,6-dihydropyridin-3-yl)pyrazolo[1,5-a]pyrazin-4-yl)-4-methyl-2-oxopyrrolidine-3-carbonitrile

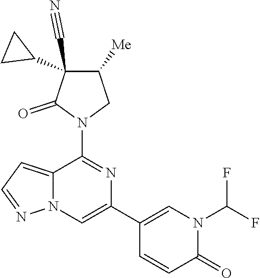 [SMILES: C[C@@H]1CN(c2nc(-c3ccc(=O)n(C(F)F)c3)cn3nccc23)C(=O)[C@]1(C#N)C1CC1]

The mixture of 1-(difluoromethyl)-5-(4,4,5,5-tetramethyl-1,3,2-dioxaborolan-2-yl)pyridin-2(1H)-one (Preparation 116, 180 mg, 0.664 mmol), (3R,4S)-1-(6-chloropyrazolo[1,5-a]pyrazin-4-yl)-3-cyclopropyl-4-methyl-2-oxopyrrolidine-3-carbonitrile (Preparation 110, 100 mg, 0.317 mmol), K₂CO₃ (131 mg, 0.950 mmol), PEPPSi-IPr (21.6 mg, 0.032 mmol) in dioxane (10 mL) and water (2 mL) was purged with N₂ for 1 min and then stirred at 90° C. for 2 h. The reaction mixture was evaporated to dryness in vacuo and the residue was purified by prep-HPLC-3 (Gradient: 45-65%) to give (3R,4S)-3-cyclopropyl-1-(6-(1-(difluoromethyl)-6-oxo-1,6-dihydropyridin-3-yl)pyrazolo[1,5-a]pyrazin-4-yl)-4-methyl-2-oxopyrrolidine-3-carbonitrile as a white solid (65.7 mg, 49%). LCMS m/z=425.1 [M+H]⁺; ¹H NMR (500 MHz, DMSO-d₆) δ: 9.38 (s, 1H), 8.44-8.18 (m, 3H), 8.09-7.80 (m, 1H), 7.08-6.65 (m, 2H), 4.36-4.32 (m, 1H), 3.97 (t, 1H), 3.26-3.14 (m, 1H), 1.57-1.50 (m, 1H), 1.33 (d, 3H), 0.79-0.64 (m, 2H), 0.63-0.54 (m, 2H).

### Example 211

### (3R,4S)-3-cyclopropyl-1-(3-fluoro-6-(1-(methyl-d3)-1H-pyrazol-4-yl)pyrazolo[1,5-a]pyrazin-4-yl)-4-methyl-2-oxopyrrolidine-3-carbonitrile

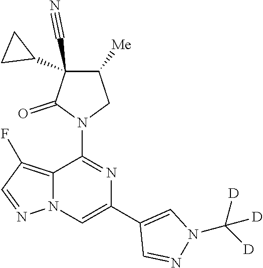 [SMILES: [2H]C([2H])([2H])n1cc(-c2cn3ncc(F)c3c(N3C[C@@H](C)[C@@](C#N)(C4CC4)C3=O)n2)cn1]

(3R,4S)-3-cyclopropyl-1-(3-fluoro-6-(1-(methyl-d3)-1H-pyrazol-4-yl)pyrazolo[1,5-a]pyrazin-4-yl)-4-methyl-2-oxopyrrolidine-3-carbonitrile was prepared as a pale yellow solid (53.2 mg) from (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125) and 1-(methyl-d3)-4-(4,4,5,5-tetramethyl-1,3,2-dioxaborolan-2-yl)-1H-pyrazole (Preparation 117) using an analogous method to that described for Example 210 except using HPLC-9 (Gradient: 5-60%). LCMS m/z=383 [M+H]⁺; ¹H NMR (600 MHz, DMSO-d₆) δ: 8.99-8.95 (m, 1H), 8.26-8.23 (m, 1H), 8.22-8.18 (m, 1H), 8.05-8.02 (m, 1H), 4.35-4.27 (m, 1H), 3.90-3.82 (m, 1H), 2.54 (s, 1H), 1.47-1.40 (m, 1H), 1.33 (d, 3H), 0.78-0.64 (m, 2H), 0.61-0.52 (m, 2H).

### Example 212

### (3R,4S)-3-cyclopropyl-1-(6-(3-fluoropyridin-2-yl)pyrazolo[1,5-a]pyrazin-4-yl)-4-methyl-2-oxopyrrolidine-3-carbonitrile

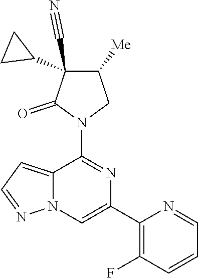 [SMILES: C[C@@H]1CN(c2nc(-c3ncccc3F)cn3nccc23)C(=O)[C@]1(C#N)C1CC1]

To a solution of (3R,4S)-1-(6-chloropyrazolo[1,5-a]pyrazin-4-yl)-3-cyclopropyl-4-methyl-2-oxopyrrolidine-3-carbonitrile (Preparation 110, 100 mg, 0.317 mmol) in dioxane (3 mL) was added 3-fluoro-2-(tributylstannyl)pyridine (367 mg, 0.950 mmol), PCy₃ (17.8 mg, 0.063 mmol), CsF (41.9 mg, 0.633 mmol) and Pd(OAc)₂ (7.1 mg, 0.032 mmol) under N₂ and the reaction stirred at 100° C. for 16 h. The mixture was quenched with aq. KF (30 mL) and extracted with EtOAc (2×20 mL). The combined organics were washed with brine (30 mL), dried (Na₂SO₄) and evaporated to dryness in vacuo. The crude was purified by prep-TLC (50% EtOAc/PE) and then further purified by prep-HPLC-3 (gradient: 39-69%) to give (3R,4S)-3-cyclopropyl-1-(6-(3-fluoropyridin-2-yl)pyrazolo[1,5-a]pyrazin-4-yl)-4-methyl-2-oxopyrrolidine-3-carbonitrile as a yellow solid (16 mg, 13%). LCMS m/z=377.1 [M+H]f; ¹H NMR (500 MHz, CDCl₃) δ: 9.08 (s, 1H), 8.55 (d, 1H), 8.11 (d, 1H), 7.57-7.52 (m, 1H), 7.39-7.34 (m, 1H), 7.17 (s, 1H), 4.37-4.32 (m, 1H), 4.00-3.95 (m, 1H), 3.15-3.09 (m, 1H), 1.43 (d, 1H), 1.22-1.20 (m, 1H), 0.84-0.71 (m, 4H).

### Example 213

### (3R,4S)-3-cyclopropyl-1-(6-(5-methoxypyridin-2-yl)pyrazolo[1,5-a]pyrazin-4-yl)-4-methyl-2-oxopyrrolidine-3-carbonitrile

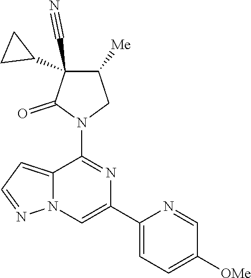 [SMILES: COc1ccc(-c2cn3nccc3c(N3C[C@@H](C)[C@@](C#N)(C4CC4)C3=O)n2)nc1]

(3R,4S)-3-Cyclopropyl-1-(6-(5-methoxypyridin-2-yl)pyrazolo[1,5-a]pyrazin-4-yl)-4-methyl-2-oxopyrrolidine-3-carbonitrile was obtained as a yellow solid (18 mg, 15%), from (3R,4S)-1-(6-chloropyrazolo[1,5-a]pyrazin-4-yl)-3-cyclopropyl-4-methyl-2-oxopyrrolidine-3-carbonitrile (Preparation 110) and 5-methoxy-2-(tributylstannyl)pyridine (Preparation 122) following the procedure described in Example 212. LCMS m/z=389.1 [M+H]⁺; ¹H NMR (500 MHz, CDCl₃) δ: 9.27 (s, 1H), 8.37 (s, 1H), 8.12 (d, 1H), 8.06 (d, 1H), 7.31 (d, 1H), 7.11 (s, 1H), 4.36-4.32 (m, 1H), 4.04-3.93 (m, 1H), 3.18-3.12 (m, 1H), 1.47 (d, 1H), 1.26-1.22 (m, 1H), 0.86-0.73 (m, 4H).

### Example 214

### (3R,4S)-3-cyclopropyl-4-methyl-1-(6-(2-methylthiazol-4-yl)pyrazolo[1,5-a]pyrazin-4-yl)-2-oxopyrrolidine-3-carbonitrile

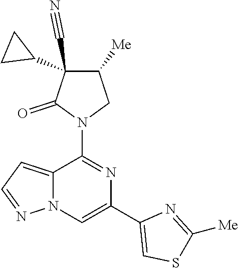 [SMILES: Cc1nc(-c2cn3nccc3c(N3C[C@@H](C)[C@@](C#N)(C4CC4)C3=O)n2)cs1]

Part 1. To a mixture of 4-bromo-2-methyl-thiazole (200 mg, 1.12 mmol), 4,4,5,5-tetramethyl-2-(4,4,5,5-tetramethyl-1,3,2-dioxaborolan-2-yl)-1,3,2-dioxaborolane (428 mg, 1.68 mmol) and KOAc (331 mg, 3.37 mmol) in dioxane (1.5 mL) was added Pd(dppf)Cl₂ (82.2 mg, 0.112 mmol) at 20° C. The mixture was stirred at 90° C. for 16 hrs and then evaporated to dryness in vacuo to give 2-methyl-4-(4,4,5,5-tetramethyl-1,3,2-dioxaborolan-2-yl)thiazole (200 mg, crude) as a black solid. It was used without further purification in Part 2.

Part 2. To a mixture of 2-methyl-4-(4,4,5,5-tetramethyl-1,3,2-dioxaborolan-2-yl)thiazole (Part 1, 200 mg, 1.40 mmol), (3R,4S)-1-(6-chloropyrazolo[1,5-a]pyrazin-4-yl)-3-cyclopropyl-4-methyl-2-oxopyrrolidine-3-carbonitrile (Preparation 110, 221 mg, 0.699 mmol) and K₃PO₄ (4451 mg, 2.10 mmol) in dioxane (1.5 mL) and water (0.3 mL) was added Pd(PtBu₃)₂ (35.8 mg, 0.07 mmol) and the reaction was stirred at 90° C. for 2 h. The reaction mixture was concentrated in vacuo and the residue purified by prep-HPLC-3 (gradient: 43-73%) to give (3R,4S)-3-cyclopropyl-4-methyl-1-(6-(2-methylthiazol-4-yl)pyrazolo[1,5-a]pyrazin-4-yl)-2-oxopyrrolidine-3-carbonitrile as a white solid (43.9 mg, 16.6% yield). LCMS m/z=379.1 [M+H]⁺; ¹H NMR (500 MHz, DMSO-d₆) δ: 8.98 (s, 1H), 8.21 (d, 1H), 8.05 (s, 1H), 7.03 (s, 1H), 4.43-4.39 (m, 1H), 3.97 (t, 1H), 3.23-3.18 (m, 1H), 2.77 (s, 3H), 1.53-1.50 (m, 1H), 1.33 (d, 3H), 0.77-0.75 (m, 1H), 0.72-0.68 (m, 1H), 0.60-0.57 (m, 2H).

### Example 215

### (3R,4S)-3-cyclopropyl-1-(6-(2,3-dihydro-1H-pyrrolo[2,3-b]pyridin-5-yl)pyrazolo[1,5-a]pyrazin-4-yl)-4-methyl-2-oxopyrrolidine-3-carbonitrile

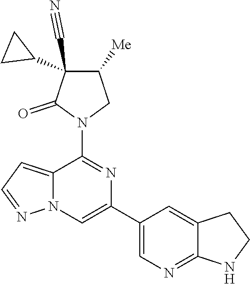 [SMILES: C[C@@H]1CN(c2nc(-c3cnc4c(c3)CCN4)cn3nccc23)C(=O)[C@]1(C#N)C1CC1]

Step A: To a solution of (1-(tert-butoxycarbonyl)-2,3-dihydro-1H-pyrrolo[2,3-b]pyridin-5-yl)boronic acid (Preparation 119, 0.4 g, 1.51 mmol) and (3R,4S)-1-(6-chloropyrazolo[1,5-a]pyrazin-4-yl)-3-cyclopropyl-4-methyl-2-oxopyrrolidine-3-carbonitrile (Preparation 110, 478 mg, 1.51 mmol) in dioxane (20 mL) and water (2 mL) was added K₃PO₄ (965 mg, 4.54 mmol) and Pd(PtBu₃)₂ (77.4 mg, 0.151 mmol) and the reaction stirred at 90° C. under N₂ for 2 h. The reaction mixture was evaporated to dryness in vacuo and the residue purified by column chromatography (SiO₂, 50% EtOAc/PE) to give tert-butyl 5-(4-((3R,4S)-3-cyano-3-cyclopropyl-4-methyl-2-oxopyrrolidin-1-yl)pyrazolo[1,5-a]pyrazin-6-yl)-2,3-dihydro-1H-pyrrolo[2,3-b]pyridine-1-carboxylate as a yellow solid (0.28 g, crude).

Step B: To a solution of tert-butyl 5-(4-((3R,4S)-3-cyano-3-cyclopropyl-4-methyl-2-oxopyrrolidin-1-yl)pyrazolo[1,5-a]pyrazin-6-yl)-2,3-dihydro-1H-pyrrolo[2,3-b]pyridine-1-carboxylate (250 mg, 0.50 mmol) in DCM (16 mL) was added HCl/EtOAc (4 M, 16 mL) and the mixture was stirred at 20° C. for 1 h. The reaction mixture was concentrated in vacuo and the residue was purified by prep HPLC-11 (gradient: 15-35%) to give (3R,4S)-3-cyclopropyl-1-(6-(2,3-dihydro-1H-pyrrolo[2,3-b]pyridin-5-yl)pyrazolo[1,5-a]pyrazin-4-yl)-4-methyl-2-oxopyrrolidine-3-carbonitrile as a yellow solid (54 mg, 27%). LCMS m/z=400.3 [M+H]⁺; ¹HNMR (400 MHz, DMSO-d₆) δ: 9.13 (s, 1H), 8.48 (s, 1H), 8.12 (t, 1H), 7.94 (s, 1H), 6.92 (d, 1H), 6.73 (s, 1H), 4.34 (t, 1H), 3.96 (t, 1H), 3.54 (t, 2H), 3.18-3.14 (m, 1H), 3.05 (t, 2H), 1.53-1.48 (m, 1H), 1.32 (d, 3H), 0.75-0.64 (m, 2H), 0.60-0.53 (m, 2H).

### Example 216

### (3R,4S)-3-cyclopropyl-4-methyl-1-(2-(1-methyl-1H-pyrazol-4-yl)pyrazolo[1,5-a]pyrazin-4-yl)-2-oxopyrrolidine-3-carbonitrile

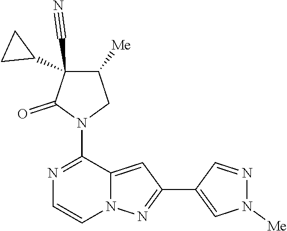 [SMILES: C[C@@H]1CN(c2nccn3nc(-c4cnn(C)c4)cc23)C(=O)[C@]1(C#N)C1CC1]

To a solution of 4-chloro-2-(1-methyl-1H-pyrazol-4-yl)pyrazolo[1,5-a]pyrazine (Preparation 148, 90 mg, 0.385 mmol) in dioxane (2.0 mL) was added (3R,4S)-3-cyclopropyl-4-methyl-2-oxopyrrolidine-3-carbonitrile (Preparation 21, 69.6 mg, 0.424 mmol), K₃PO₄ (115 mg, 0.539 mmol), Xantphos (22.3 mg, 0.039 mmol) and Pd₂(dba)₃ (35.3 mg, 0.039 mmol). The mixture was stirred at 80° C. for 2 h under N₂. The mixture was concentrated and purified by prep-HPLC-3 (gradient: 30-60%) to afford (3R,4S)-3-cyclopropyl-4-methyl-1-(2-(1-methyl-1H-pyrazol-4-yl)pyrazolo[1,5-a]pyrazin-4-yl)-2-oxopyrrolidine-3-carbonitrile as a white solid (108 mg, 78%). LCMS m/z=326.1 [M+H]⁺; ¹H NMR (400 MHz, CDCl₃) δ: 8.24 (d, 1H), 7.96 (s, 1H), 7.86 (s, 1H), 7.54 (d, 1H), 7.08 (s, 1H), 4.11-4.16 (m, 1H), 3.91-4.00 (m, 4H), 3.06-3.13 (m, 1H), 1.44 (d, 3H), 1.17-1.23 (m, 1H), 0.73-0.83 (m, 4H).

### Example 217

### (3R,4S)-1-(6-(1-cyclopentyl-1H-pyrazol-4-yl)-3-fluoropyrazolo[1,5-a]pyrazin-4-yl)-3-cyclopropyl-4-methyl-2-oxopyrrolidine-3-carbonitrile

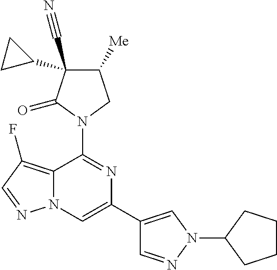 [SMILES: C[C@@H]1CN(c2nc(-c3cnn(C4CCCC4)c3)cn3ncc(F)c23)C(=O)[C@]1(C#N)C1CC1]

To a solution of (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 100 mg, 0.300 mmol) in dioxane (4 mL) and water (1 mL) was added 1-cyclopentyl-4-(4,4,5,5-tetramethyl-1,3,2-dioxaborolan-2-yl)pyrazole (78.6 mg, 0.300 mmol), K₂CO₃ (41.4 mg, 0.300 mmol) and PEPPSI-IPr catalyst (204 mg, 0.300 mmol) and the mixture stirred at 100° C. for 16 h. The reaction mixture was partitioned between EtOAc and brine. The layers separated and the combined organics dried (Na₂SO₄) and evaporated to dryness in vacuo. The residue was purified using reverse phase chromatography (Waters Sunfire OBD 100×50 mm, 5 mm; 95% water/5% ACN in 0.1% TFA). LCMS m/z=434.2 [M+H]⁺; ¹H NMR (500 MHz, DMSO-d₆) δ: 9.01-8.94 (m, 1H), 8.37-8.30 (m, 1H), 8.25-8.19 (m, 1H), 8.05 (s, 1H), 4.81-4.65 (m, 2H), 4.37-4.29 (m, 1H), 3.87 (br t, 1H), 3.21-3.10 (m, 1H), 2.99 (s, 2H), 2.15-2.06 (m, 1H), 2.00-1.89 (m, 2H), 1.86-1.75 (m, 2H), 1.71-1.58 (m, 2H), 1.45 (br s, 1H), 1.32 (d, 2H), 0.78-0.65 (m, 2H), 0.59-0.53 (m, 1H).

### Example 218-223

The title compounds were prepared from (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125) and the appropriate boronate ester (RBY) using an analogous method to that described for Example 217

| Example No | Name/Structure/RBY/Data |
|---|---|
| 218 | (3R,4S)-3-cyclopropyl-1-(3-fluoro-6-(1-(tetrahydro-2H-pyran-4-yl)-1H- |
| | pyrazol-4-yl)pyrazolo[1,5-a]pyrazin-4-yl)-4-methyl-2-oxopyrrolidine-3- |
| | carbonitrile |
| | 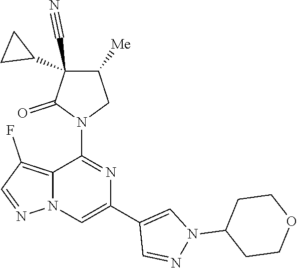 [SMILES: C[C@@H]1CN(c2nc(-c3cnn(C4CCOCC4)c3)cn3ncc(F)c23)C(=O)[C@]1(C#N)C1CC1] |
| | RBY: 1-(tetrahydro-2H-pyran-4-yl)-4-(4,4,5,5-tetramethyl-1,3,2-dioxaborolan-2- |
| | yl)-1H-pyrazole |
| | LCMS m/z = 449.1 [M]⁺; ¹H NMR (500 MHz, DMSO-d₆) δ: 8.99 (d, 1H), |
| | 8.38 (s, 1H), 8.21 (br d, 1H), 8.09 (d, 1H), 4.47-4.41 (m, 1H), 4.49-4.43 (m, |
| | 1H), 4.36-4.31 (m, 2H), 3.98 (dt, 4H), 3.89-3.85 (m, 1H), 3.51-3.46 (m, |
| | 1H), 1.46-1.42 (m, 1H), 1.33 (s, 3H), 0.77-0.73 (m, 2H), 0.71-0.65 (m, 2H), |
| | 0.57 (d, 2H). |
| 219 | (3R,4S)-1-(6-(1-cyclohexyl-1H-pyrazol-4-yl)-3-fluoropyrazolo[1,5- |
| | a]pyrazin-4-yl)-3-cyclopropyl-4-methyl-2-oxopyrrolidine-3-carbonitrile |
| | 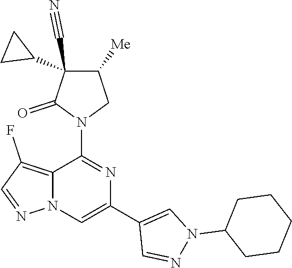 [SMILES: C[C@@H]1CN(c2nc(-c3cnn(C4CCCCC4)c3)cn3ncc(F)c23)C(=O)[C@]1(C#N)C1CC1] |
| | RBY: 1-cyclohexyl-4-(4,4,5,5-tetramethyl-1,3,2-dioxaborolan-2-yl)-1H- |
| | pyrazole |
| | LCMS m/z = 447.2 [M]⁺; ¹H NMR (500 MHz, DMSO-d₆) δ: 9.01-8.94 (m, |
| | 1H), 8.35-8.28 (m, 1H), 8.21 (br d, 1H), 8.08-7.99 (m, 1H), 8.03-7.98 (m, |
| | 1H), 4.33 (br dd, 2H), 4.23-4.12 (m, 2H), 3.88 (br t, 2H), 3.17 (br s, 1H), |
| | 2.11-2.02 (m, 2H), 1.86-1.80 (m, 1H), 1.77-1.65 (m, 3H), 1.44 (br d, 2H), |
| | 1.33 (d, 2H), 1.24 (br s, 1H), 0.77-0.72 (m, 1H), 0.69 (br d, 1H), 0.60-0.52 |
| | (m, 1H). |
| 220 | (3R,4S)-1-(6-(1-(cyanomethyl)-1H-pyrazol-4-yl)-3-fluoropyrazolo[1,5- |
| | a]pyrazin-4-yl)-3-cyclopropyl-4-methyl-2-oxopyrrolidine-3-carbonitrile |
| | 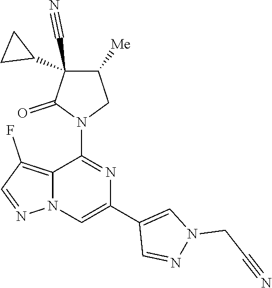 [SMILES: C[C@@H]1CN(c2nc(-c3cnn(CC#N)c3)cn3ncc(F)c23)C(=O)[C@]1(C#N)C1CC1] |
| | RBY: 2-(4-(4,4,5,5-tetramethyl-1,3,2-dioxaborolan-2-yl)-1H-pyrazol-1- |
| | yl)acetonitrile |
| | LCMS m/z = 405.2 [M + H]⁺; ¹H NMR (500 MHz, DMSO-d₆) δ: 9.09 (d, |
| | 1H), 8.86-8.81 (m, 1H), 8.42 (s, 1H), 8.25 (d, 1H), 5.57 (s, 2H), 4.34-4.30 |
| | (m, 1H), 3.89-3.85 (m, 1H), 1.48-1.43 (m, 1H), 1.09 (br s, 1H), 1.05 (d, |
| | 3H), 0.78-0.72 (m, 1H), 0.70-0.66 (m, 1H), 0.59-0.54 (m, 2H). |
| 221 | (3R,4S)-3-cyclopropyl-1-(6-(1,5-dimethyl-1H-pyrazol-4-yl)-3- |
| | fluoropyrazolo[1,5-a]pyrazin-4-yl)-4-methyl-2-oxopyrrolidine-3- |
| | carbonitrile |
| | 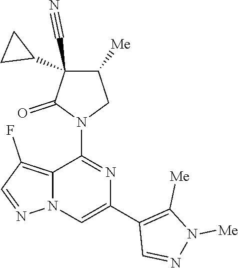 [SMILES: Cc1c(-c2cn3ncc(F)c3c(N3C[C@@H](C)[C@@](C#N)(C4CC4)C3=O)n2)cnn1C] |
| | RBY: 1,5-dimethyl-4-(4,4,5,5-tetramethyl-1,3,2-dioxaborolan-2-yl)-1H- |
| | pyrazole |
| | LCMS m/z = 393.2 [M]⁺; ¹H NMR (500 MHz, DMSO-d₆) δ: 8.85-8.82 (m, |
| | 1H), 8.23 (br d, 1H), 7.92 (s, 1H), 4.26 (br dd, 2H), 3.85 (s, 1H), 3.82-3.74 |
| | (m, 3H), 1.46 (br s, 2H), 1.36-1.29 (m, 3H), 1.25 (br d, 2H), 0.76-0.72 (m, |
| | 1H), 0.70-0.65 (m, 1H), 0.56 (dt, 2H). |
| 222 | (3R,4S)-3-cyclopropyl-1-(3-fluoro-6-(pyridin-3-yl)pyrazolo[1,5-a]pyrazin- |
| | 4-yl)-4-methyl-2-oxopyrrolidine-3-carbonitrile |
| | 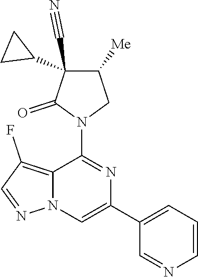 [SMILES: C[C@@H]1CN(c2nc(-c3cccnc3)cn3ncc(F)c23)C(=O)[C@]1(C#N)C1CC1] |
| | RBY: 3-(4-(4,4,5,5-tetramethyl-1,3,2-dioxaborolan-2-yl)-1H-pyrazol-1- |
| | yl)pyridine |
| | LCMS m/z = 376,1 [M + H]⁺; ¹H NMR (500 MHz, DMSO-d₆) δ: 9.45-9.40 |
| | (m, 1H), 9.34-9.27 (m, 1H), 8.63 (br dd, 1H), 8.47 (br dd, 1H), 8.35 (br d, |
| | 1H), 7.56-7.51 (m, 1H), 4.33 (br d, 1H), 3.94-3.84 (m, 2H), 2.80 (br d, 1H), |
| | 1.51-1.46 (m, 1H), 1.33 (d, 1H), 1.25 (br d, 1H), 1.21 (br d, 1H), 1.10-1.02 |
| | (m, 2H). |
| 223 | (3R,4S)-3-cyclopropyl-1-(3-fluoro-6-(5-methoxypyridin-3-yl)pyrazolo[1,5- |
| | a]pyrazin-4-yl)-4-methyl-2-oxopyrrolidine-3-carbonitrile |
| | 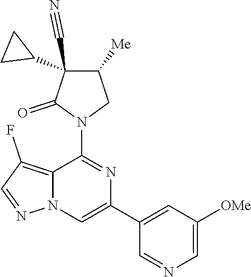 [SMILES: COc1cncc(-c2cn3ncc(F)c3c(N3C[C@@H](C)[C@@](C#N)(C4CC4)C3=O)n2)c1] |
| | RBY: 3-methoxy-5-(4-(4,4,5,5-tetramethyl-1,3,2-dioxaborolan-2-yl)-1H- |
| | pyrazol-1-yl)pyridine |
| | LCMS m/z = 406.1 [M]⁺; ¹H NMR (500 MHz, DMSO-d₆) δ: 9.46 (br d, |
| | J1H), 8.93 (br d, 1H), 8.37-8.32 (m, 2H), 8.03 (d, 1H), 4.38-4.34 (m, 1H), |
| | 3.95-3.92 (m, 3H), 1.49 (br s, 1H), 1.35-1.31 (m, 3H), 0.75 (br s, 2H), 0.68 |
| | (br s, 2H), 0.58 (br d, 2H). |

### Example 224-267

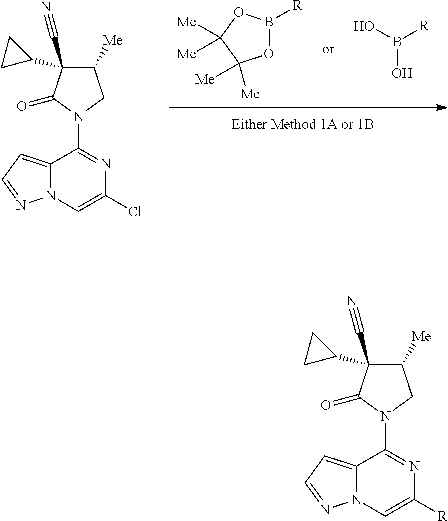

The title compounds were prepared using a one-step library protocol described below using Method 1A or Method 1B as noted in the table.

Method 1A: To a solution of (3R,4S)-1-(6-chloropyrazolo[1,5-a]pyrazin-4-yl)-3-cyclopropyl-4-methyl-2-oxopyrrolidine-3-carbonitrile (Preparation 110, 48 mg, 0.152 mmol, 1.00 eq) and the appropriate boronate ester or boronic acid (0.183 mmol, 1.20 eq) in dioxane (4 mL), H₂O (0.15 mL) was added K₃PO₄ (64.4 mg, 0.304 mmol, 2.00 eq) and Pd(dppf)Cl₂·DCM (6.53 mg, 0.008 mmol, 0.05 eq) under N₂. The mixture was stirred at 100° C. for 16 h. Water (5 mL) was added to the reaction mixture and extracted with EtOAc (3×5 mL). The combined organics were evaporated to dryness by speedvac and the residue purified by prep-HPLC-15 or 16; gradient optimised for each example to give the title compound.

Method 1B: To a solution of To a solution of (3R,4S)-1-(6-chloropyrazolo[1,5-a]pyrazin-4-yl)-3-cyclopropyl-4-methyl-2-oxopyrrolidine-3-carbonitrile (Preparation 110, 48 mg, 0.152 mmol, 1.00 eq) and the appropriate boronate ester or boronic acid (0.183 mmol, 1.20 eq) in dioxane (1 mL), H₂O (0.10 mL) was added K₃PO₄ (64.4 mg, 0.304 mmol, 2.00 eq) and Pd-132 (5.66 mg, 0.008 mmol, 0.05 eq) under N₂. The mixture was stirred at 100° C. for 16 h. Water (5 mL) was added to the reaction mixture and extracted with EtOAc (3×5 mL). The combined organics were evaporated to dryness by speedvac and the residue purified by prep-HPLC-15 or 16; gradient optimised for each example to give the title compound.

| Exa- | |
|---|---|
| mple | |
| No. | Name/Structure/Reactants/Method/HPLC/Data |
| 224 | (3R,4S)-3-cyclopropyl-1-(6-(6-methoxypyridin- |
| | 3-yl)pyrazolo[1,5-a]pyrazin-4- |
| | yl)-4-methyl-2-oxopyrrolidine-3-carbonitrile |
| | 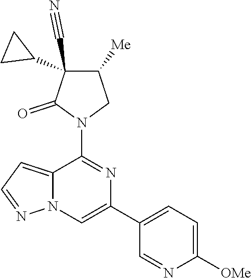 [SMILES: COc1ccc(-c2cn3nccc3c(N3C[C@@H](C)[C@@](C#N)(C4CC4)C3=O)n2)cn1] |
| | Boronate/Boronic acid: (6-methoxypyridin- |
| | 3-yl)boronic acid |
| | Method 1A; prep-HPLC-16 |
| | LCMS m/z = 389.2 [M + H]⁺ |
| 225 | (3R,4S)-3-cyclopropyl-4-methyl-1-(6-(1- |
| | methyl-1H-indazol-5-yl)pyrazolo[1,5- |
| | a]pyrazin-4-yl)-2-oxopyrrolidine-3-carbonitrile |
| | trifluoroacetate |
| | 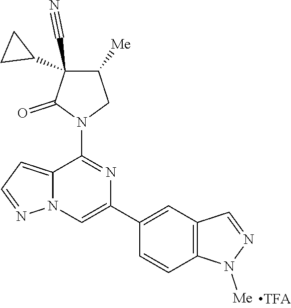 [SMILES: C[C@@H]1CN(c2nc(-c3ccc4c(cnn4C)c3)cn3nccc23)C(=O)[C@]1(C#N)C1CC1] |
| | Boronate/Boronic acid: (1-methyl-1H-indazol- |
| | 5-yl)boronic acid |
| | Method 1A; prep-HPLC-15 |
| | LCMS m/z = 412.2 [M + H]⁺ |
| 226 | (3R,4S)-3-cyclopropyl-1-(6-(2-fluoropyridin- |
| | 3-yl)pyrazolo[1,5-a]pyrazin-4- |
| | yl)-4-methyl-2-oxopyrrolidine-3-carbonitrile |
| | trifluoroacetate |
| | 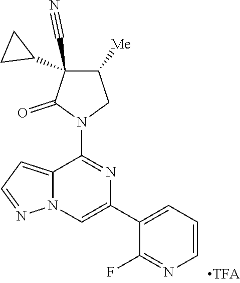 [SMILES: C[C@@H]1CN(c2nc(-c3cccnc3F)cn3nccc23)C(=O)[C@]1(C#N)C1CC1] |
| | Boronate/Boronic acid: 2-fluoro-3-(4,4,5, |
| | 5-tetramethyl-1,3,2-dioxaborolan-2- |
| | yl)pyridine |
| | Method 1A; prep-HPLC-15 |
| | LCMS m/z = 377.1 [M + H]⁺ |
| 227 | 5-(4-((3R,4S)-3-cyano-3-cyclopropyl-4- |
| | methyl-2-oxopyrrolidin-1- |
| | yl)pyrazolo[1,5-a]pyrazin-6-yl)-N- |
| | methylpicolinamide trifluoroacetate |
| | 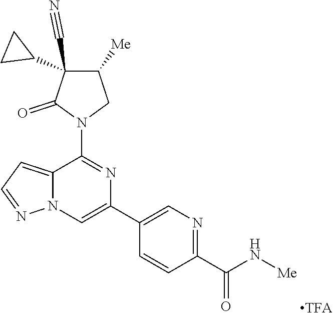 [SMILES: CNC(=O)c1ccc(-c2cn3nccc3c(N3C[C@@H](C)[C@@](C#N)(C4CC4)C3=O)n2)cn1] |
| | Boronate/Boronic acid: N-methyl-5-(4,4,5,5-tetramethyl- |
| | 1,3,2-dioxaborolan-2- |
| | yl)picolinamide |
| | Method 1A; prep-HPLC-15 |
| | LCMS m/z = 416.2 [M + H]⁺ |
| 228 | N-(4-(4-((3R,4S)-3-cyano-3-cyclopropyl-4-methyl-2- |
| | oxopyrrolidin-1- |
| | yl)pyrazolo[1,5-a]pyrazin-6-yl)pyridin-2-yl)acetamide |
| | 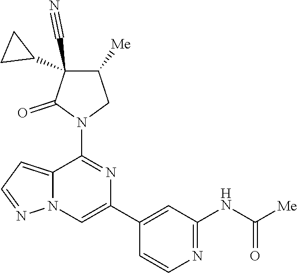 [SMILES: CC(=O)Nc1cc(-c2cn3nccc3c(N3C[C@@H](C)[C@@](C#N)(C4CC4)C3=O)n2)ccn1] |
| | Boronate/Boronic acid: N-(4-(4,4,5,5-tetramethyl- |
| | 1,3,2-dioxaborolan-2- |
| | yl)pyridin-2-yl)acetamide |
| | Method 1A; prep-HPLC-16 |
| | LCMS m/z = 416.2 [M + H]⁺ |
| 229 | (3R,4S)-3-cyclopropyl-4-methyl-1-(6-(1-methyl- |
| | 1H-pyrazol-3-yl)pyrazolo[1,5- |
| | a]pyrazin-4-yl)-2-oxopyrrolidine-3-carbonitrile |
| | trifluoroacetate |
| | 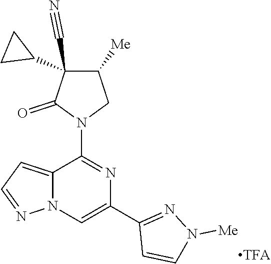 [SMILES: C[C@@H]1CN(c2nc(-c3ccn(C)n3)cn3nccc23)C(=O)[C@]1(C#N)C1CC1] |
| | Boronate/Boronic acid: 1-methyl-3-(4,4,5,5- |
| | tetramethyl-1,3,2-dioxaborolan-2- |
| | yl)-1H-pyrazole |
| | Method 1A; prep-HPLC-15 |
| | LCMS m/z = 362.2 [M + H]⁺ |
| 230 | (3R,4S)-1-(6-(1H-pyrrolo[2,3-b]pyridin-5- |
| | yl)pyrazolo[1,5-a]pyrazin-4-yl)-3- |
| | cyclopropyl-4-methyl-2-oxopyrrolidine-3- |
| | carbonitrile |
| | 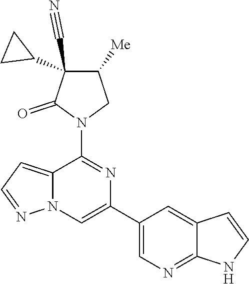 [SMILES: C[C@@H]1CN(c2nc(-c3cnc4[nH]ccc4c3)cn3nccc23)C(=O)[C@]1(C#N)C1CC1] |
| | Boronate/Boronic acid: (1H-pyrrolo[2, |
| | 3-b]pyridin-5-yl)boronic acid |
| | Method 1A; prep-HPLC-16 |
| | LCMS m/z = 398.2 [M + H]⁺ |
| 231 | (3R,4S)-3-cyclopropyl-1-(6-(2-methoxy- |
| | pyridin-4-yl)pyrazolo[1,5-a]pyrazin-4- |
| | yl)-4-methyl-2-oxopyrrolidine-3-carbonitrile |
| | trifluoroacetate |
| | 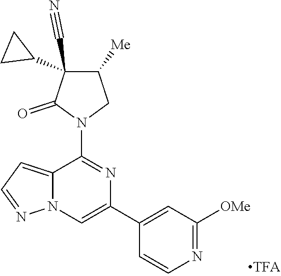 [SMILES: COc1cc(-c2cn3nccc3c(N3C[C@@H](C)[C@@](C#N)(C4CC4)C3=O)n2)ccn1] |
| | Boronate/Boronic acid: (2-methoxypyridin-4-yl) |
| | boronic acid |
| | Method 1A; prep-HPLC-15 |
| | LCMS m/z = 389.2 [M + H]⁺ |
| 232 | (3R,4S)-1-(6-(1H-pyrazol-3-yl)pyrazolo[1, |
| | 5-a]pyrazin-4-yl)-3-cyclopropyl-4- |
| | methyl-2-oxopyrrolidine-3-carbonitrile |
| | trifluoroacetate |
| | 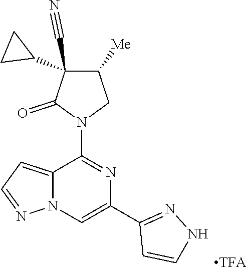 [SMILES: C[C@@H]1CN(c2nc(-c3cc[nH]n3)cn3nccc23)C(=O)[C@]1(C#N)C1CC1] |
| | Boronate/Boronic acid: 3-(4,4,5,5- |
| | tetramethyl-1,3,2-dioxaborolan-2-yl)-1H- |
| | pyrazole |
| | Method 1A; prep-HPLC-15 |
| | LCMS m/z = 348.2 [M + H]⁺ |
| 233 | (3R,4S)-3-cyclopropyl-4-methyl-1-(6-(6- |
| | methylpyridin-3-yl)pyrazolo[1,5- |
| | a]pyrazin-4-yl)-2-oxopyrrolidine-3-carbonitrile |
| | trifluoroacetate |
| | 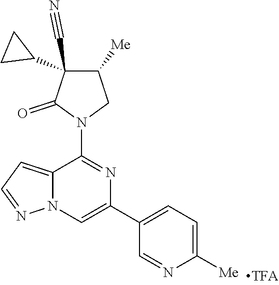 [SMILES: Cc1ccc(-c2cn3nccc3c(N3C[C@@H](C)[C@@](C#N)(C4CC4)C3=O)n2)cn1] |
| | Boronate/Boronic acid: (6-methylpyridin-3- |
| | yl)boronic acid |
| | Method 1A; prep-HPLC-15 |
| | LCMS m/z = 373.2 [M + H]⁺ |
| 234 | (3R,4S)-3-cyclopropyl-4-methyl-2-oxo-1-(6- |
| | (2-(trifluoromethyl)pyridin-4- |
| | yl)pyrazolo[1,5-a]pyrazin-4-yl)pyrrolidine- |
| | 3-carbonitrile trifluoroacetate |
| | 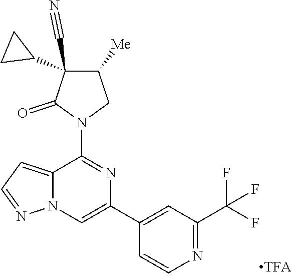 [SMILES: C[C@@H]1CN(c2nc(-c3ccnc(C(F)(F)F)c3)cn3nccc23)C(=O)[C@]1(C#N)C1CC1] |
| | Boronate/Boronic acid: 4-(4,4,5,5-tetramethyl- |
| | 1,3,2-dioxaborolan-2-yl)-2- |
| | (trifluoromethyl)pyridine |
| | Method 1A; prep-HPLC-15 |
| | LCMS m/z = 427.2 [M + H]⁺ |
| 235 | (3R,4S)-3-cyclopropyl-4-methyl-1-(6-(4-methyl-3- |
| | oxo-3,4-dihydro-2H- |
| | pyrido[3,2-b][1,4]oxazin-7-yl)pyrazolo[1, |
| | 5-a]pyrazin-4-yl)-2-oxopyrrolidine-3- |
| | carbonitrile trifluoroacetate |
| | 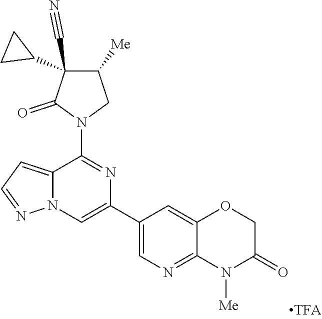 [SMILES: C[C@@H]1CN(c2nc(-c3cnc4c(c3)OCC(=O)N4C)cn3nccc23)C(=O)[C@]1(C#N)C1CC1] |
| | Boronate/Boronic acid: 4-methyl-7-(4,4,5,5-tetramethyl- |
| | 1,3,2-dioxaborolan-2- |
| | yl)-2H-pyrido[3,2-b][1,4]oxazin-3(4H)-one |
| | Method 1A; prep-HPLC-15 |
| | LCMS m/z = 444.2 [M + H]⁺ |
| 236 | (3R,4S)-3-cyclopropyl-4-methyl-1-(6-(5-methylpyridin- |
| | 3-yl)pyrazolo[1,5- |
| | a]pyrazin-4-yl)-2-oxopyrrolidine-3-carbonitrile |
| | 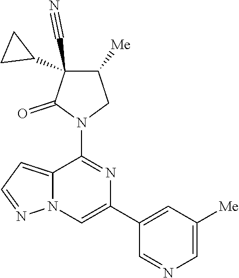 [SMILES: Cc1cncc(-c2cn3nccc3c(N3C[C@@H](C)[C@@](C#N)(C4CC4)C3=O)n2)c1] |
| | Boronate/Boronic acid: (5-methylpyridin- |
| | 3-yl)boronic acid |
| | Method 1A; ; prep-HPLC-16 |
| | LCMS m/z = 373.2 [M + H]⁺ |
| 237 | (3R,4S)-3-cyclopropyl-1-(6-(2-fluoropyridin- |
| | 4-yl)pyrazolo[1,5-a]pyrazin-4- |
| | yl)-4-methyl-2-oxopyrrolidine-3-carbonitrile |
| | trifluoroacetate |
| | 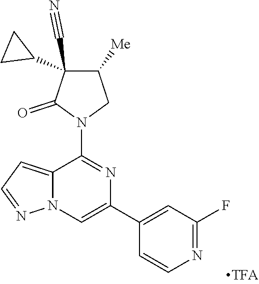 [SMILES: C[C@@H]1CN(c2nc(-c3ccnc(F)c3)cn3nccc23)C(=O)[C@]1(C#N)C1CC1] |
| | Boronate/Boronic acid: (2-fluoropyridin-4- |
| | yl)boronic acid |
| | Method 1A; prep-HPLC-15 |
| | LCMS m/z = 377.1 [M + H]⁺ |
| 238 | (3R,4S)-3-cyclopropyl-4-methyl-2-oxo-1- |
| | (6-(6-(trifluoromethyl)pyridin-3- |
| | yl)pyrazolo[1,5-a]pyrazin-4-yl)pyrrolidine- |
| | 3-carbonitrile trifluoroacetate |
| | 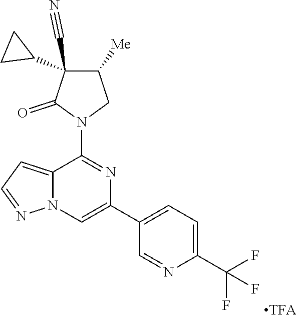 [SMILES: C[C@@H]1CN(c2nc(-c3ccc(C(F)(F)F)nc3)cn3nccc23)C(=O)[C@]1(C#N)C1CC1] |
| | Boronate/Boronic acid: (6-(trifluoromethyl)pyridin- |
| | 3-yl)boronic acid |
| | Method 1A; prep-HPLC-15 |
| | LCMS m/z = 427.1 [M + H]⁺ |
| 239 | (3R,4S)-3-cyclopropyl-4-methyl-1-(6-(2- |
| | methylthiazol-5-yl)pyrazolo[1,5- |
| | a]pyrazin-4-yl)-2-oxopyrrolidine-3- |
| | carbonitrile |
| | 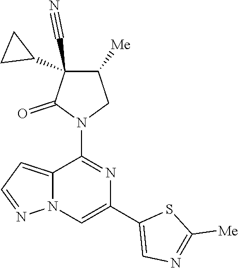 [SMILES: Cc1ncc(-c2cn3nccc3c(N3C[C@@H](C)[C@@](C#N)(C4CC4)C3=O)n2)s1] |
| | Boronate/Boronic acid: 2-methyl-5- |
| | (4,4,5,5-tetramethyl-1,3,2-dioxaborolan-2- |
| | yl)thiazole |
| | Method 1A; ; prep-HPLC-16 |
| | LCMS m/z = 379.1 [M + H]⁺ |
| 240 | (3R,4S)-3-cyclopropyl-1-(6-(2- |
| | methoxypyrimidin-5-yl)pyrazolo[1,5-a]pyrazin- |
| | 4-yl)-4-methyl-2-oxopyrrolidine-3-carbonitrile |
| | trifluoroacetate |
| | 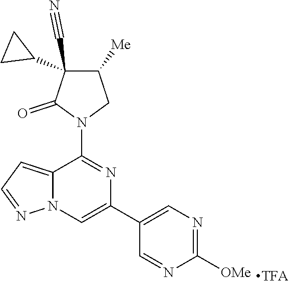 [SMILES: COc1ncc(-c2cn3nccc3c(N3C[C@@H](C)[C@@](C#N)(C4CC4)C3=O)n2)cn1] |
| | Boronate/Boronic acid: (2-methoxypyrimidin-5- |
| | yl)boronic acid |
| | Method 1A; prep-HPLC-15 |
| | LCMS m/z = 390.2 [M + H]⁺ |
| 241 | (3R,4S)-3-cyclopropyl-4-methyl-1-(6-(2- |
| | methylpyridin-4-yl)pyrazolo[1,5- |
| | a]pyrazin-4-yl)-2-oxopyrrolidine-3-carbonitrile |
| | trifluoroacetate |
| | 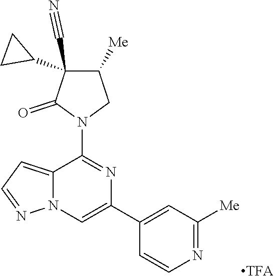 [SMILES: Cc1cc(-c2cn3nccc3c(N3C[C@@H](C)[C@@](C#N)(C4CC4)C3=O)n2)ccn1] |
| | Boronate/Boronic acid: (2-methylpyridin-4- |
| | yl)boronic acid |
| | Method 1A; prep-HPLC-15 |
| | LCMS m/z = 373.2 [M + H]⁺ |
| 242 | (3R,4S)-3-cyclopropyl-4-methyl-1-(6-(1- |
| | methyl-1H-indazol-7-yl)pyrazolo[1,5- |
| | a]pyrazin-4-yl)-2-oxopyrrolidine-3- |
| | carbonitrile |
| | 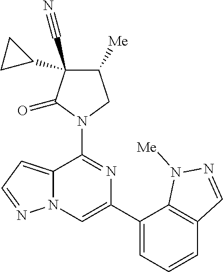 [SMILES: C[C@@H]1CN(c2nc(-c3cccc4cnn(C)c34)cn3nccc23)C(=O)[C@]1(C#N)C1CC1] |
| | Boronate/Boronic acid: (1-methyl-1H- |
| | indazol-7-yl)boronic acid |
| | Method 1A; ; prep-HPLC-16 |
| | LCMS m/z = 412.2 [M + H]⁺ |
| 243 | (3R,4S)-3-cyclopropyl-1-(6-(6- |
| | fluoropyridin-3-yl)pyrazolo[1,5-a]pyrazin-4- |
| | yl)-4-methyl-2-oxopyrrolidine-3-carbonitrile |
| | trifluoroacetate |
| | 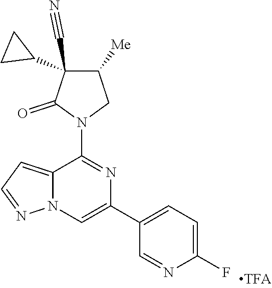 [SMILES: C[C@@H]1CN(c2nc(-c3ccc(F)nc3)cn3nccc23)C(=O)[C@]1(C#N)C1CC1] |
| | Boronate/Boronic acid: (6-fluoropyridin-3- |
| | yl)boronic acid |
| | Method 1A; prep-HPLC-15 |
| | LCMS m/z = 377.1 [M + H]⁺ |
| 244 | (3R,4S)-3-cyclopropyl-1-(6-(5-methoxypyridin- |
| | 3-yl)pyrazolo[1,5-a]pyrazin-4- |
| | yl)-4-methyl-2-oxopyrrolidine-3-carbonitrile |
| | trifluoroacetate |
| | 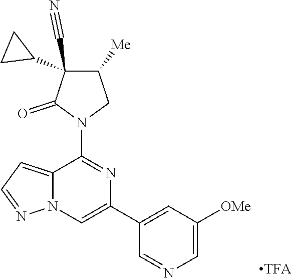 [SMILES: COc1cncc(-c2cn3nccc3c(N3C[C@@H](C)[C@@](C#N)(C4CC4)C3=O)n2)c1] |
| | Boronate/Boronic acid: (5-methoxypyridin-3- |
| | yl)boronic acid |
| | Method 1A; prep-HPLC-15 |
| | LCMS m/z = 389.2 [M + H]⁺ |
| 245 | (3R,4S)-3-cyclopropyl-4-methyl-1-(6-(1-methyl- |
| | 6-oxo-1,6-dihydropyridin-3- |
| | yl)pyrazolo[1,5-a]pyrazin-4-yl)-2-oxopyrrolidine- |
| | 3-carbonitrile |
| | 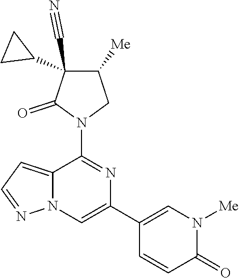 [SMILES: C[C@@H]1CN(c2nc(-c3ccc(=O)n(C)c3)cn3nccc23)C(=O)[C@]1(C#N)C1CC1] |
| | Boronate/Boronic acid: 1-methyl-5- |
| | (4,4,5,5-tetramethyl-1,3,2-dioxaborolan-2- |
| | yl)pyridin-2(1H)-one |
| | Method 1A |
| | LCMS m/z = 389.1 [M + H]⁺ |
| 246 | (3R,4S)-3-cyclopropyl-1-(6-(5- |
| | fluoropyridin-3-yl)pyrazolo[1,5-a]pyrazin-4- |
| | yl)-4-methyl-2-oxopyrrolidine-3-carbonitrile |
| | trifluoroacetate |
| | 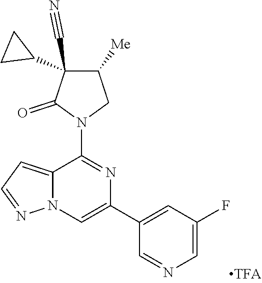 [SMILES: C[C@@H]1CN(c2nc(-c3cncc(F)c3)cn3nccc23)C(=O)[C@]1(C#N)C1CC1] |
| | Boronate/Boronic acid: (5-fluoropyridin- |
| | 3-yl)boronic acid |
| | Method 1A; prep-HPLC-15 |
| | LCMS m/z = 377.2 [M + H]⁺ |
| 247 | (3R,4S)-1-(6-(1H-imidazol-2-yl)pyrazolo |
| | [1,5-a]pyrazin-4-yl)-3-cyclopropyl-4- |
| | methyl-2-oxopyrrolidine-3-carbonitrile |
| | trifluoroacetate |
| | 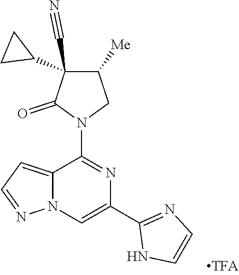 [SMILES: C[C@@H]1CN(c2nc(-c3ncc[nH]3)cn3nccc23)C(=O)[C@]1(C#N)C1CC1] |
| | Boronate/Boronic acid: (1H-imidazol-2- |
| | yl)boronic acid hydrochloride |
| | Method 1A; prep-HPLC-15 |
| | LCMS m/z = 348.1 [M + H]⁺ |
| 248 | (3R,4S)-1-(6-(5-chloropyridin-3-yl) |
| | pyrazolo[1,5-a]pyrazin-4-yl)-3- |
| | cyclopropyl-4-methyl-2-oxopyrrolidine-3- |
| | carbonitrile trifluoroacetate |
| | 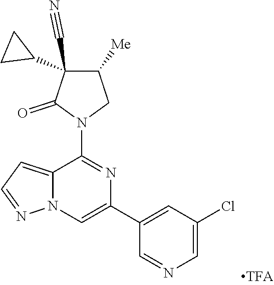 [SMILES: C[C@@H]1CN(c2nc(-c3cncc(Cl)c3)cn3nccc23)C(=O)[C@]1(C#N)C1CC1] |
| | Boronate/Boronic acid: (5-chloropyridin-3- |
| | yl)boronic acid |
| | Method 1A; prep-HPLC-15 |
| | LCMS m/z = 393.1 [M + H]⁺ |
| 249 | N-(5-(4-((3R,4S)-3-cyano-3-cyclopropyl-4- |
| | methyl-2-oxopyrrolidin-1- |
| | yl)pyrazolo[1,5-a]pyrazin-6-yl)pyridin-3-yl)acetamide |
| | trifluoroacetate |
| | 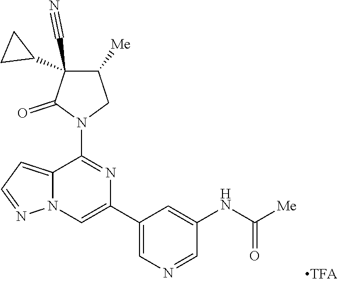 [SMILES: CC(=O)Nc1cncc(-c2cn3nccc3c(N3C[C@@H](C)[C@@](C#N)(C4CC4)C3=O)n2)c1] |
| | Boronate/Boronic acid: N-(5-(4,4,5,5- |
| | tetramethyl-1,3,2-dioxaborolan-2- |
| | yl)pyridin-3-yl)acetamide |
| | Method 1A; prep-HPLC-15 |
| | LCMS m/z = 416.2 [M + H]⁺ |
| 250 | (3R,4S)-3-cyclopropyl-1-(6-(2- |
| | methoxypyridin-3-yl)pyrazolo[1, |
| | 5-a]pyrazin-4-yl)-4-methyl-2- |
| | oxopyrrolidine-3-carbonitrile |
| | 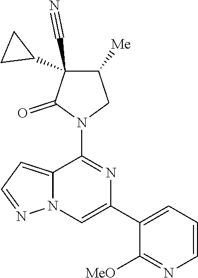 [SMILES: COc1ncccc1-c1cn2nccc2c(N2C[C@@H](C)[C@@](C#N)(C3CC3)C2=O)n1] |
| | Boronate/Boronic acid: (2- |
| | methoxypyridin-3-yl)boronic acid |
| | Method 1A; prep-HPLC-16 |
| | LCMS m/z = 389.2 [M + H]⁺ |
| 251 | (3R,4S)-3-cyclopropyl-4-methyl-1-(6-(1- |
| | (oxetan-3-yl)-1H-pyrazol-4- |
| | yl)pyrazolo[1,5-a]pyrazin-4-yl)-2- |
| | oxopyrrolidine-3-carbonitrile |
| | 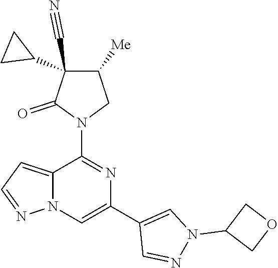 [SMILES: C[C@@H]1CN(c2nc(-c3cnn(C4COC4)c3)cn3nccc23)C(=O)[C@]1(C#N)C1CC1] |
| | Boronate/Boronic acid: 1-(oxetan-3-yl)-4- |
| | (4,4,5,5-tetramethyl-1,3,2- |
| | dioxaborolan-2-yl)-1H-pyrazole |
| | Method 1A; prep-HPLC-16 |
| | LCMS m/z = 404.2 [M + H]⁺ |
| 252 | 4-(4-((3R,4S)-3-cyano-3-cyclopropyl-4-methyl- |
| | 2-oxopyrrolidin-1- |
| | yl)pyrazolo[1,5-a]pyrazin-6-yl)-N- |
| | methylpicolinamide trifluoroacetate |
| | 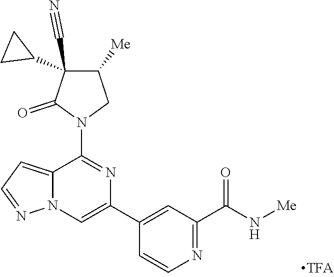 [SMILES: CNC(=O)c1cc(-c2cn3nccc3c(N3C[C@@H](C)[C@@](C#N)(C4CC4)C3=O)n2)ccn1] |
| | Boronate/Boronic acid: N-methyl-4-(4,4,5,5-tetramethyl- |
| | 1,3,2-dioxaborolan-2- |
| | yl)picolinamide |
| | Method 1A; prep-HPLC-15 |
| | LCMS m/z = 416.2 [M + H]⁺ |
| 253 | (3R,4S)-3-cyclopropyl-1-(6-(3,6-dimethoxypyridazin- |
| | 4-yl)pyrazolo[1,5- |
| | a]pyrazin-4-yl)-4-methyl-2-oxopyrrolidine-3- |
| | carbonitrile trifluoroacetate |
| | 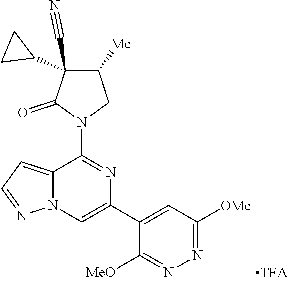 [SMILES: COc1cc(-c2cn3nccc3c(N3C[C@@H](C)[C@@](C#N)(C4CC4)C3=O)n2)c(OC)nn1] |
| | Boronate/Boronic acid: (3,6-dimethoxypyridazin- |
| | 4-yl)boronic acid |
| | Method 1A; prep-HPLC-15 |
| | LCMS m/z = 420.1 [M + H]⁺ |
| | Method 1B − Pd-132 |
| 254 | (3R,4S)-3-cyclopropyl-4-methyl-1-(6-(1-methyl- |
| | 1H-pyrazol-5-yl)pyrazolo[1,5- |
| | a]pyrazin-4-yl)-2-oxopyrrolidine-3- |
| | carbonitrile trifluoroacetate |
| | 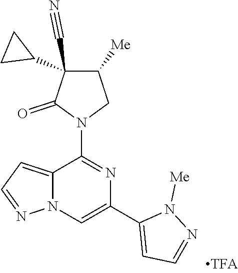 [SMILES: C[C@@H]1CN(c2nc(-c3ccnn3C)cn3nccc23)C(=O)[C@]1(C#N)C1CC1] |
| | Boronate/Boronic acid: (1-methyl-1H- |
| | pyrazol-5-yl)boronic acid |
| | Method 1B; prep-HPLC-15 |
| | LCMS m/z = 362.2 [M + H]⁺ |
| 255 | (3R,4S)-3-cyclopropyl-1-(6-(6-fluoropyridin-2- |
| | yl)pyrazolo[1,5-a]pyrazin-4- |
| | yl)-4-methyl-2-oxopyrrolidine-3-carbonitrile |
| | trifluoroacetate |
| | 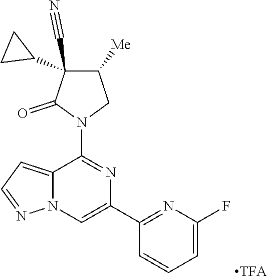 [SMILES: C[C@@H]1CN(c2nc(-c3cccc(F)n3)cn3nccc23)C(=O)[C@]1(C#N)C1CC1] |
| | Boronate/Boronic acid: (6-fluoropyridin-2- |
| | yl)boronic acid |
| | Method 1B; prep-HPLC-15 |
| | LCMS m/z = 377.2 [M + H]⁺ |
| 256 | (3R,4S)-3-cyclopropyl-1-(6-(2-(difluoromethoxy) |
| | pyridin-3-yl)pyrazolo[1,5- |
| | a]pyrazin-4-yl)-4-methyl-2-oxopyrrolidine-3- |
| | carbonitrile trifluoroacetate |
| | 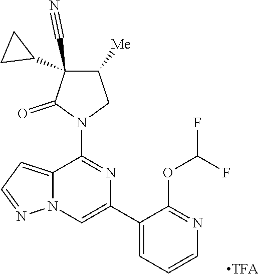 [SMILES: C[C@@H]1CN(c2nc(-c3cccnc3OC(F)F)cn3nccc23)C(=O)[C@]1(C#N)C1CC1] |
| | Boronate/Boronic acid: (2-(difluoromethoxy) |
| | pyridin-3-yl)boronic acid |
| | Method 1B; prep-HPLC-15 |
| | LCMS m/z = 425.2 [M + H]⁺ |
| 257 | (3R,4S)-3-cyclopropyl-4-methyl-1-(6-(6- |
| | (methylsulfonyl)pyridin-3- |
| | yl)pyrazolo[1,5-a]pyrazin-4-yl)-2-oxopyrrolidine-3- |
| | carbonitrile trifluoroacetate |
| | 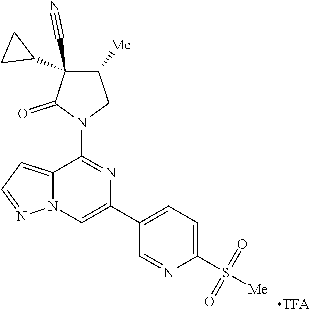 [SMILES: C[C@@H]1CN(c2nc(-c3ccc(S(C)(=O)=O)nc3)cn3nccc23)C(=O)[C@]1(C#N)C1CC1] |
| | Boronate/Boronic acid: (6-(methylsulfonyl)pyridin- |
| | 3-yl)boronic acid |
| | Method 1B; prep-HPLC-15 |
| | LCMS m/z = 437.2 [M + H]⁺ |
| 258 | (3R,4S)-3-cyclopropyl-1-(6-(5-hydroxypyridin-3- |
| | yl)pyrazolo[1,5-a]pyrazin-4- |
| | yl)-4-methyl-2-oxopyrrolidine-3-carbonitrile |
| | trifluoroacetate |
| | 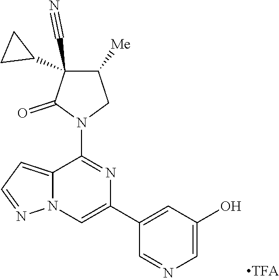 [SMILES: C[C@@H]1CN(c2nc(-c3cncc(O)c3)cn3nccc23)C(=O)[C@]1(C#N)C1CC1] |
| | Boronate/Boronic acid: 5-(4,4,5,5-tetramethyl- |
| | 1,3,2-dioxaborolan-2-yl)pyridin- |
| | 3-ol |
| | Method 1B; prep-HPLC-15 |
| | LCMS m/z = 375.1 [M + H]⁺ |
| 259 | (3R,4S)-3-cyclopropyl-4-methyl-1-(6-(1- |
| | methyl-1H-indazol-6-yl)pyrazolo[1,5- |
| | a]pyrazin-4-yl)-2-oxopyrrolidine-3-carbonitrile |
| | trifluoroacetate |
| | 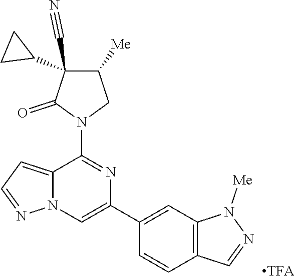 [SMILES: C[C@@H]1CN(c2nc(-c3ccc4cnn(C)c4c3)cn3nccc23)C(=O)[C@]1(C#N)C1CC1] |
| | Boronate/Boronic acid: 1-methyl-6-(4,4,5,5- |
| | tetramethyl-1,3,2-dioxaborolan-2- |
| | yl)-1H-indazole |
| | Method 1B; prep-HPLC-15 |
| | LCMS m/z = 412.3 [M + H]⁺ |
| 260 | (3R,4S)-3-cyclopropyl-4-methyl-2-oxo-1-(6-(5- |
| | (trifluoromethyl)pyridin-3- |
| | yl)pyrazolo[1,5-a]pyrazin-4-yl)pyrrolidine-3- |
| | carbonitrile trifluoroacetate |
| | 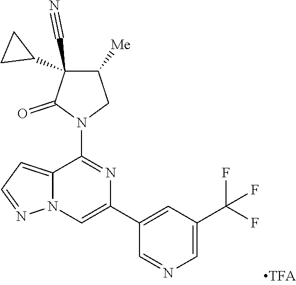 [SMILES: C[C@@H]1CN(c2nc(-c3cncc(C(F)(F)F)c3)cn3nccc23)C(=O)[C@]1(C#N)C1CC1] |
| | Boronate/Boronic acid: 3-(4,4,5,5-tetramethyl- |
| | 1,3,2-dioxaborolan-2-yl)-5- |
| | (trifluoromethyl)pyridine |
| | Method 1B; prep-HPLC-15 |
| | LCMS m/z = 427.1 [M + H]⁺ |
| 261 | (3R,4S)-3-cyclopropyl-4-methyl-1-(6-(1- |
| | methyl-2-oxo-1,2-dihydropyridin-4- |
| | yl)pyrazolo[1,5-a]pyrazin-4-yl)-2-oxopyrrolidine- |
| | 3-carbonitrile trifluoroacetate |
| | 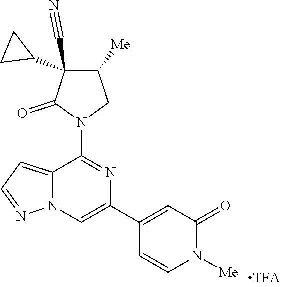 [SMILES: C[C@@H]1CN(c2nc(-c3ccn(C)c(=O)c3)cn3nccc23)C(=O)[C@]1(C#N)C1CC1] |
| | Boronate/Boronic acid: 1-methyl-4-(4,4,5,5- |
| | tetramethyl-1,3,2-dioxaborolan-2- |
| | yl)pyridin-2(1H)-one |
| | Method 1B; prep-HPLC-15 |
| | LCMS m/z = 389.2 [M + H]⁺ |
| 262 | 5-(4-((3R,4S)-3-cyano-3-cyclopropyl-4-methyl- |
| | 2-oxopyrrolidin-1- |
| | yl)pyrazolo[1,5-a]pyrazin-6-yl)-2- |
| | methoxynicotinonitrile trifluoroacetate |
| | 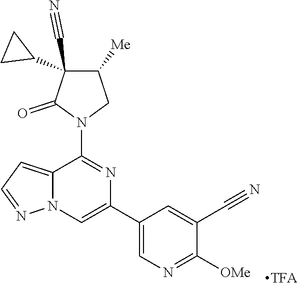 [SMILES: COc1ncc(-c2cn3nccc3c(N3C[C@@H](C)[C@@](C#N)(C4CC4)C3=O)n2)cc1C#N] |
| | Boronate/Boronic acid: 2-methoxy-5-(4,4,5,5- |
| | tetramethyl-1,3,2-dioxaborolan- |
| | 2-yl)nicotinonitrile |
| | Method 1B; prep-HPLC-15 |
| | LCMS m/z = 414.2 [M + H]⁺ |
| 263 | (3R,4S)-3-cyclopropyl-1-(6-(imidazo[1, |
| | 2-a]pyridin-6-yl)pyrazolo[1,5- |
| | a]pyrazin-4-yl)-4-methyl-2-oxopyrrolidine-3- |
| | carbonitrile trifluoroacetate |
| | 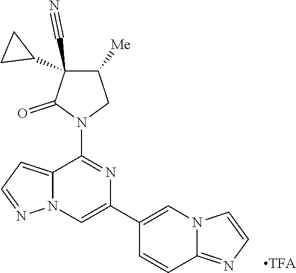 [SMILES: C[C@@H]1CN(c2nc(-c3ccc4nccn4c3)cn3nccc23)C(=O)[C@]1(C#N)C1CC1] |
| | Boronate/Boronic acid: imidazo[1,2-a]pyridin- |
| | 6-ylboronic acid |
| | Method 1B; prep-HPLC-15 |
| | LCMS m/z = 398.1 [M + H]⁺ |
| 264 | (3R,4S)-3-cyclopropyl-1-(6-(2-(dimethylamino) |
| | pyrimidin-5-yl)pyrazolo[1,5- |
| | a]pyrazin-4-yl)-4-methyl-2-oxopyrrolidine-3- |
| | carbonitrile trifluoroacetate |
| | 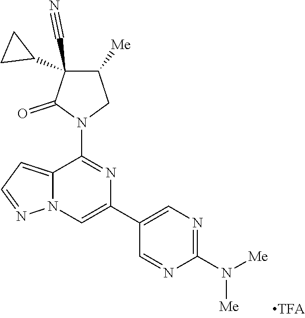 [SMILES: C[C@@H]1CN(c2nc(-c3cnc(N(C)C)nc3)cn3nccc23)C(=O)[C@]1(C#N)C1CC1] |
| | Boronate/Boronic acid: (2-(dimethylamino)pyrimidin- |
| | 5-yl)boronic acid |
| | Method 1B; prep-HPLC-15 |
| | LCMS m/z = 403.2 [M + H]⁺ |
| 265 | (3R,4S)-3-cyclopropyl-4-methyl-2-oxo-1-(6-(pyrimidin- |
| | 5-yl)pyrazolo[1,5- |
| | a]pyrazin-4-yl)pyrrolidine-3-carbonitrile |
| | trifluoroacetate |
| | 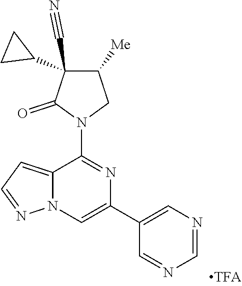 [SMILES: C[C@@H]1CN(c2nc(-c3cncnc3)cn3nccc23)C(=O)[C@]1(C#N)C1CC1] |
| | Boronate/Boronic acid: pyrimidin-5- |
| | ylboronic acid |
| | Method 1B; prep-HPLC-15 |
| | LCMS m/z = 360.1 [M + H]⁺ |
| 266 | tert-butyl (5-(4-((3R,4S)-3-cyano-3-cyclopropyl-4- |
| | methyl-2-oxopyrrolidin-1- |
| | yl)pyrazolo[1,5-a]pyrazin-6-yl)pyridin-2-yl)(methyl)carbamate |
| | trifluoroacetate |
| | 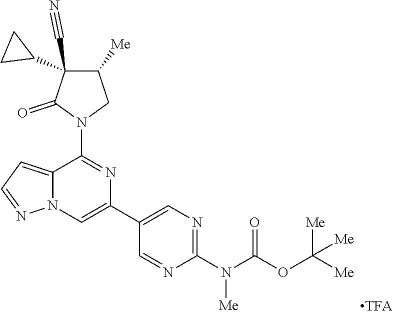 [SMILES: C[C@@H]1CN(c2nc(-c3cnc(N(C)C(=O)OC(C)(C)C)nc3)cn3nccc23)C(=O)[C@]1(C#N)C1CC1] |
| | Boronate/Boronic acid: tert-butyl methyl(5-(4,4,5,5-tetramethyl- |
| | 1,3,2- |
| | dioxaborolan-2-yl)pyridin-2-yl)carbamate |
| | Method 1B; prep-HPLC-15 |
| | LCMS m/z = 488.3 [M + H]⁺ |
| 267 | (3R,4S)-3-cyclopropyl-1-(6-(6-hydroxypyridin-3- |
| | yl)pyrazolo[1,5-a]pyrazin-4- |
| | yl)-4-methyl-2-oxopyrrolidine-3-carbonitrile |
| | trifluoroacetate |
| | 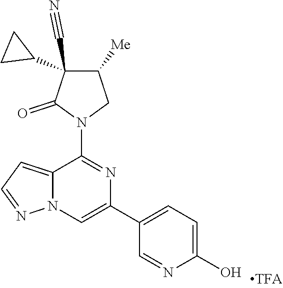 [SMILES: C[C@@H]1CN(c2nc(-c3ccc(O)nc3)cn3nccc23)C(=O)[C@]1(C#N)C1CC1] |
| | Boronate/Boronic acid: (6-hydroxypyridin-3- |
| | yl)boronic acid |
| | Method 1B; prep-HPLC-15 |
| | LCMS m/z = 375.1 [M + H]⁺ |

### Example 268

### (3R,4S)-3-cyclopropyl-4-methyl-1-(2-(1-methyl-1H-pyrazol-4-yl)furo[3,2-b]pyridin-7-yl)-2-oxopyrrolidine-3-carbonitrile

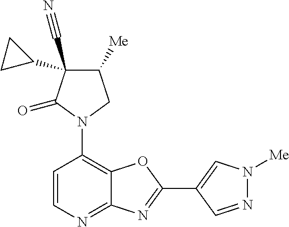 [SMILES: C[C@@H]1CN(c2ccnc3nc(-c4cnn(C)c4)oc23)C(=O)[C@]1(C#N)C1CC1]

To a solution of 7-chloro-2-(1-methyl-1H-pyrazol-4-yl)furo[3,2-b]pyridine (Preparation 143, 120 mg, 0.514 mmol) and (3R,4S)-3-cyclopropyl-4-methyl-2-oxopyrrolidine-3-carbonitrile (Preparation 21, 211 mg, 1.28 mmol) in dioxane (5 mL) was added RuPhos Pd G3 (42.9 mg, 0.051 mmol) and K₂CO₃ (142 mg, 1.03 mmol) and the mixture stirred under N₂ at 100° C. for 2 h under microwave irradiation. The mixture was evaporated to dryness in vacuo and the residue purified by prep-HPLC-17 (gradient: 25-45%) to give a residue that was further purified by prep-HPLC-3 (Gradient: 28-48%) to afford (3R,4S)-3-cyclopropyl-4-methyl-1-(2-(1-methyl-1H-pyrazol-4-yl)furo[3,2-b]pyridin-7-yl)-2-oxopyrrolidine-3-carbonitrile as a white solid (28 mg, 15%). LCMS m/z=362.1 [M+H]⁺; ¹H NMR (400 MHz, CDCl₃) δ: 8.44 (d, 1H), 7.86 (s, 1H), 7.79-7.76 (m, 2H), 6.88 (s, 1H), 4.40-4.35 (m, 1H), 4.02-3.97 (m, 4H), 3.16-3.06 (m, 1H), 1.45 (d, 3H), 1.20-1.15 (m, 1H), 0.84-0.74 (m, 4H).

### Example 269

### (3R,4S)-3-cyclopropyl-4-methyl-1-(7-(1-methyl-1H-pyrazol-4-yl)imidazo[1,2-c]pyrimidin-5-yl)-2-oxopyrrolidine-3-carbonitrile

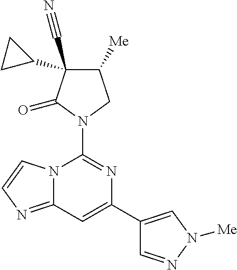 [SMILES: C[C@@H]1CN(c2nc(-c3cnn(C)c3)cc3nccn23)C(=O)[C@]1(C#N)C1CC1]

To a solution of (3R,4S)-3-cyclopropyl-4-methyl-2-oxopyrrolidine-3-carbonitrile (Preparation 21, 84.3 mg, 0.514 mmol) and 5-chloro-7-(1-methyl-1H-pyrazol-4-yl)imidazo[1,2-c]pyrimidine (Preparation 153, 60 mg, 0.257 mmol) in dioxane (5 mL) was added K₃PO₄ (109 mg, 0.514 mmol), Xantphos (14.9 mg, 0.026 mmol) and Pd₂dba₃ (23.5 mg, 0.26 mmol) at 25° C. The mixture was stirred at 70° C. for 16 h under N₂. The mixture was concentrated and purified by column chromatography (SiO₂; 0-10% MeOH/DCM) give a white solid which was purified by prep-HPLC-3 (Gradient: 29-59%) to afford (3R,4S)-3-cyclopropyl-4-methyl-1-(7-(1-methyl-1H-pyrazol-4-yl)imidazo[1,2-c]pyrimidin-5-yl)-2-oxopyrrolidine-3-carbonitrile as a white solid (6.3 mg, 6.8%). LCMS m/z=362.1 [M+H]⁺; ¹H NMR (400 MHz, CDCl₃) δ: 7.95 (s, 1H), 7.86 (s, 1H), 7.64 (s, 1H), 7.53 (s, 1H), 7.44 (s, 1H), 4.25-4.20 (m, 1H), 4.03-3.97 (m, 4H), 3.26-3.16 (m, 1H), 1.47 (d, 3H), 1.28-1.24 (m, 1H), 0.86-0.80 (m, 4H).

### Example 270

### (3R,4S)-3-cyclopropyl-1-(6-(1-(difluoromethyl)-1H-pyrazol-4-yl)-3-fluoropyrazolo[1,5-a]pyridin-4-yl)-4-methyl-2-oxopyrrolidine-3-carbonitrile

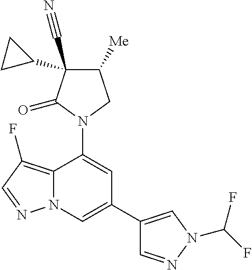 [SMILES: C[C@@H]1CN(c2cc(-c3cnn(C(F)F)c3)cn3ncc(F)c23)C(=O)[C@]1(C#N)C1CC1]

To a solution of 6-(1-(difluoromethyl)-1H-pyrazol-4-yl)-3-fluoropyrazolo[1,5-a]pyridin-4-yl trifluoromethanesulfonate (Preparation 157, 23.8 mg, 0.059 mmol) in toluene (2 mL) was added (3R,4S)-3-cyclopropyl-4-methyl-2-oxopyrrolidine-3-carbonitrile (Preparation 21, 14.7 mg, 0.089 mmol), Xantphos (3.44 mg, 5.95 □mol), Pd₂(dba)₃ (5.5 mg, 5.95 □mol) and K₂CO₃ (16.4 mg, 0.119 mmol) and the resulting mixture stirred at 100° C. for 1 h under N₂. The reaction mixture was concentrated in vacuo and the residue purified by prep-HPLC-3 (Gradient: 38-68%) to afford (3R,4S)-3-cyclopropyl-1-(6-(1-(difluoromethyl)-1H-pyrazol-4-yl)-3-fluoropyrazolo[1,5-a]pyridin-4-yl)-4-methyl-2-oxopyrrolidine-3-carbonitrile as a white solid (16.8 mg, 68%). LCMS m/z=415.1 [M+H]⁺; ¹H NMR (400 MHz, CDCl₃) δ: 8.43 (s, 1H), 8.09 (s, 1H), 7.92 (s, 1H), 7.86 (d, 1H), 7.24 (t, 1H), 7.21 (s, 1H), 3.95-3.90 (m, 1H), 3.68-3.74 (m, 1H), 3.17-3.14 (m, 1H), 1.43 (d, 1H), 1.22-1.20 (m, 1H), 0.89-0.78 (m, 4H).

### Example 271

### (3R,4S)-3-cyclopropyl-4-methyl-1-(6-(1-methyl-1H-pyrazol-4-yl)pyrazolo[1,5-a]pyridin-4-yl)-2-oxopyrrolidine-3-carbonitrile

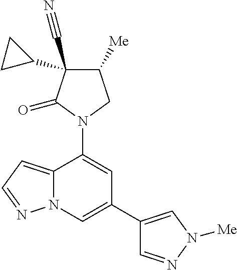 [SMILES: C[C@@H]1CN(c2cc(-c3cnn(C)c3)cn3nccc23)C(=O)[C@]1(C#N)C1CC1]

A mixture of 6-(1-methyl-1H-pyrazol-4-yl)pyrazolo[1,5-a]pyridin-4-yl trifluoromethanesulfonate (Preparation 163, 120 mg, 0.347 mmol), (3R,4S)-3-cyclopropyl-4-methyl-2-oxopyrrolidine-3-carbonitrile (Preparation 21, 68.3 mg, 0.416 mmol), XantPhos G3 (35.8 mg, 0.035 mmol), K₃PO₄ (110 mg, 0.520 mmol) was degassed under an atmosphere of N₂ and the mixture stirred at 70° C. for 48 h. The reaction was diluted with EtOAc and washed with NaHCO₃, H₂O, and brine. The combined organics were dried (Na₂SO₄) and evaporated to dryness in vacuo. The residue was purified by HPLC-9 (Gradient: 5-55%) and purified by reversed phase chromatography (C-18) to afford (3R,4S)-3-cyclopropyl-4-methyl-1-(6-(1-methyl-1H-pyrazol-4-yl)pyrazolo[1,5-a]pyridin-4-yl)-2-oxopyrrolidine-3-carbonitrile as a white solid (11.6 mg). LCMS m/z=361.1 [M+H]⁺; ¹H NMR (600 MHz, DMSO-d₆) δ: 9.04-8.95 (m, 1H), 8.26-8.24 (m, 1H), 8.03-8.0 (m, 1H), 8.00-7.97 (m, 1H), 7.61-7.60 (m, 1H), 7.61-7.58 (m, 1H), 6.59-6.56 (m, 1H), 4.01-3.96 (m, 1H), 3.92-3.87 (m, 3H), 3.87-3.82 (m, 1H), 1.53-1.47 (m, 1H), 1.32-1.29 (m, 3H), 0.79-0.68 (m, 2H), 0.60-0.54 (m, 2H).

### Example 272

### (3R,4S)-3-cyclopropyl-4-methyl-1-(2-(1-methyl-1H-pyrazol-4-yl)pyrazolo[1,5-a]pyridin-4-yl)-2-oxopyrrolidine-3-carbonitrile

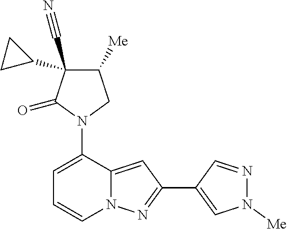 [SMILES: C[C@@H]1CN(c2cccn3nc(-c4cnn(C)c4)cc23)C(=O)[C@]1(C#N)C1CC1]

To a mixture of 2-(1-methyl-1H-pyrazol-4-yl)pyrazolo[1,5-a]pyridin-4-yl trifluoromethanesulfonate (Preparation 172, 80 mg, 0.231 mmol), (3R,4S)-3-cyclopropyl-4-methyl-2-oxopyrrolidine-3-carbonitrile (Preparation 21, 41.7 mg, 0.254 mmol) and K₂CO₃ (63.9 mg, 0.462 mmol) in toluene (2 mL) was added Pd₂(dba)₃ (21.2 mg, 0.023 mmol) and Xantphos (13.4 mg, 0.023 mmol) and the resulting mixture stirred at 80° C. for 3 h. The mixture was concentrated in vacuo and the residue purified by prep-HPLC-3 (Gradient: 30-60%) to give (3R,4S)-3-cyclopropyl-4-methyl-1-(2-(1-methyl-1H-pyrazol-4-yl)pyrazolo[1,5-a]pyridin-4-yl)-2-oxopyrrolidine-3-carbonitrile as a white solid (11.6 mg, 14%). LCMS m/z=361.1 [M+H]⁺; ¹H NMR (500 MHz, CDCl₃) δ: 8.38 (d, 1H), 7.91 (s, 1H), 7.84 (s, 1H), 7.13 (d, 1H), 6.75-6.72 (m, 1H), 6.47 (s, 1H), 3.99 (s, 3H), 3.97-3.93 (m, 1H), 3.69 (t, 1H), 3.17-3.11 (m, 1H), 1.44 (d, 3H), 1.22-1.18 (m, 1H), 0.85-0.84 (m, 2H), 0.81-0.77 (m, 2H).

### Example 273

### (3R,4S)-3-cyclopropyl-4-methyl-1-(2-(1-methyl-1H-pyrazol-4-yl)pyrazolo[1,5-a]pyrimidin-7-yl)-2-oxopyrrolidine-3-carbonitrile

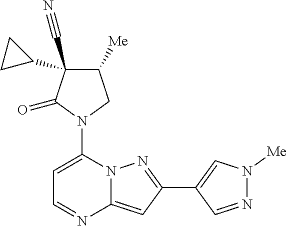 [SMILES: C[C@@H]1CN(c2ccnc3cc(-c4cnn(C)c4)nn23)C(=O)[C@]1(C#N)C1CC1]

A solution of (3R,4S)-3-cyclopropyl-4-methyl-2-oxopyrrolidine-3-carbonitrile (Preparation 21, 77.3 mg, 0.471 mmol), NaH (17.2 mg, 0.428 mmol, 60% purity) in DMF (2 mL) was stirred at 25° C. for 10 min. To this was added 7-chloro-2-(1-methyl-1H-pyrazol-4-yl)pyrazolo[1,5-a]pyrimidine (Preparation 176, 100 mg, 0.428 mmol) and the resulting mixture and stirred at 25° C. for 1 h. The reaction was quenched with water (50 mL) and extracted with EtOAc (3×30 mL). The combined organics were washed with brine (30 mL), dried (Na₂SO₄) and concentrated under vacuum. The residue was purified by prep-HPLC-3 (Gradient: 31-61%) to give (3R,4S)-3-cyclopropyl-4-methyl-1-(2-(1-methyl-1H-pyrazol-4-yl)pyrazolo[1,5-a]pyrimidin-7-yl)-2-oxopyrrolidine-3-carbonitrile as a white solid (50 mg, 32%). LCMS m/z=362.2 [M+H]⁺; ¹H NMR (500 MHz, CDCl₃) δ: 8.44 (d, 1H), 7.93 (s, 1H), 7.83 (s, 1H), 7.04 (d, 1H), 6.77 (s, 1H), 4.26-4.35 (m, 2H), 3.97 (s, 3H), 3.09-3.16 (m, 1H), 1.46 (d, 3H), 1.23-1.27 (m, 1H), 0.76-0.93 (m, 4H).

### Example 274

### (3R,4S)-3-cyclopropyl-4-methyl-1-(5-(1-methyl-1H-pyrazol-4-yl)-1H-pyrazolo[3,4-b]pyridin-3-yl)-2-oxopyrrolidine-3-carbonitrile

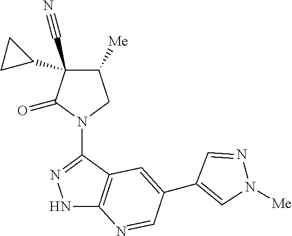 [SMILES: C[C@@H]1CN(c2n[nH]c3ncc(-c4cnn(C)c4)cc23)C(=O)[C@]1(C#N)C1CC1]

To a solution of (3R,4S)-3-cyclopropyl-4-methyl-1-(5-(1-methyl-1H-pyrazol-4-yl)-1-((2-(trimethylsilyl)ethoxy)methyl)-1H-pyrazolo[3,4-b]pyridin-3-yl)-2-oxopyrrolidine-3-carbonitrile (Preparation 179, 50 mg, 0.102 mmol) in DCM (5 mL) was added TFA (2 mL) and the resulting mixture stirred at 20° C. for 18 h. The mixture was concentrated and the residue purified by prep-HPLC-3 (Gradient: 23-53%) to give (3R,4S)-3-cyclopropyl-4-methyl-1-(5-(1-methyl-1H-pyrazol-4-yl)-1H-pyrazolo[3,4-b]pyridin-3-yl)-2-oxopyrrolidine-3-carbonitrile as a white solid (17.5 mg, 48%). LCMS m/z=362.2 [M+H]⁺; ¹H NMR (500 MHz, CDCl₃) δ: 10.69 (br s, 1H), 8.96 (s, 1H), 8.73 (s, 1H), 7.84 (s, 1H), 7.76 (s, 1H), 4.26-4.22 (m, 1H), 3.99 (s, 3H), 3.82-3.77 (m, 1H), 3.18-3.12 (m, 1H), 1.44 (d, 3H), 1.18-1.16 (m, 1H), 0.82-0.73 (m, 4H).

### Example 275

### (3R,4S)-3-cyclopropyl-4-methyl-1-(1-(1-methyl-1H-pyrazol-4-yl)-1H-pyrazolo[3,4-c]pyridin-3-yl)-2-oxopyrrolidine-3-carbonitrile

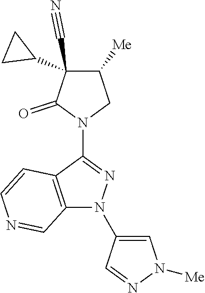 [SMILES: C[C@@H]1CN(c2nn(-c3cnn(C)c3)c3cnccc23)C(=O)[C@]1(C#N)C1CC1]

To a solution of 3-bromo-1-(1-methyl-1H-pyrazol-4-yl)-1H-pyrazolo[3,4-c]pyridine (60.0 mg, 215.74 umol) and (3R,4S)-3-cyclopropyl-4-methyl-2-oxopyrrolidine-3-carbonitrile (Preparation 21, 39 mg, 0.237 mmol) in DMSO (3 mL) was added CuI (4.1 mg, 0.022 mmol), N¹,N²-dimethylethane-1,2-diamine (1.6 mg, 0.022 mmol) and t-BuOK (48.4 mg, 0.431 mmol) under N₂ and the mixture stirred at 110° C. for 1 h under microwave irradiation. The mixture was purified by prep-HPLC-2 (gradient: 37-67%) to give (3R,4S)-3-cyclopropyl-4-methyl-1-(1-(1-methyl-1H-pyrazol-4-yl)-1H-pyrazolo[3,4-c]pyridin-3-yl)-2-oxopyrrolidine-3-carbonitrile as a white solid (51 mg, 65%). LCMS m/z=362.2 [M+H]⁺; ¹H NMR (400 MHz, DMSO-d₆) δ: 9.16 (s, 1H), 8.41 (s, 1H), 8.33 (s, 1H), 8.14-8.12 (m, 1H), 7.95 (s, 1H), 4.22-4.17 (m, 1H), 3.93 (s, 3H), 3.83-3.77 (m, 1H), 3.24-3.17 (m, 1H), 1.49-1.46 (m, 1H), 1.29 (d, 3H), 0.72-0.51 (m, 4H).

### Example 276

### (3R,4S)-3-cyclopropyl-1-(1-(1-(difluoromethyl)-1H-pyrazol-4-yl)-1H-pyrazolo[3,4-c]pyridin-3-yl)-4-methyl-2-oxopyrrolidine-3-carbonitrile

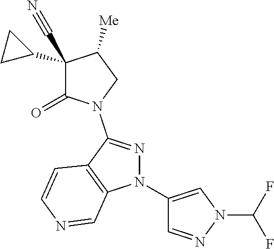 [SMILES: C[C@@H]1CN(c2nn(-c3cnn(C(F)F)c3)c3cnccc23)C(=O)[C@]1(C#N)C1CC1]

(3R,4S)-3-cyclopropyl-1-(1-(1-(difluoromethyl)-1H-pyrazol-4-yl)-1H-pyrazolo[3,4-c]pyridin-3-yl)-4-methyl-2-oxopyrrolidine-3-carbonitrile was prepared as a yellow solid (25 mg, 28%) from 3-bromo-1-(1-(difluoromethyl)-1H-pyrazol-4-yl)-1H-pyrazolo[3,4-c]pyridine (Preparation 182) and (3R,4S)-3-cyclopropyl-4-methyl-2-oxopyrrolidine-3-carbonitrile (Preparation 21) using an analogous method to that described for Example 275. LCMS m/z=398.0 [M+H]⁺; ¹H NMR (400 MHz, CDCl₃) δ: 9.08 (br s, 1H), 8.42 (br s, 2H), 8.23 (s, 1H), 8.08 (s, 1H), 7.27 (t, 1H), 4.29-4.23 (m, 1H), 3.85-3.79 (m, 1H), 3.20-3.11 (m, 1H), 1.44 (d, 3H), 1.21-1.15 (m, 1H), 0.84-0.74 (m, 4H).

### Example 277

### (3R,4S)-3-cyclopropyl-1-(1-(1-ethyl-1H-pyrazol-4-yl)-1H-pyrazolo[3,4-c]pyridin-3-yl)-4-methyl-2-oxopyrrolidine-3-carbonitrile

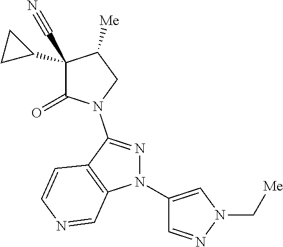 [SMILES: CCn1cc(-n2nc(N3C[C@@H](C)[C@@](C#N)(C4CC4)C3=O)c3ccncc32)cn1]

To a solution of 3-bromo-1-(1-ethyl-1H-pyrazol-4-yl)-1H-pyrazolo[3,4-c]pyridine (Preparation 181, 80 mg, 0.274 mmol) and (3R,4S)-3-cyclopropyl-4-methyl-2-oxopyrrolidine-3-carbonitrile (Preparation 21, 49.5 mg, 0.301 mmol) in dioxane (3 mL) was added CuI (5.2 mg, 0.027 mmol), N¹,N²-dimethylethane-1,2-diamine (2.4 mg, 0.027 mmol) and K₃PO₄ (116 mg, 0.548 mmol) under N₂ and the mixture stirred at 110° C. for 20 h. The mixture was concentrated and purified by prep-HPLC-2 (gradient: 44-74%) to give the (3R,4S)-3-cyclopropyl-1-(1-(1-ethyl-1H-pyrazol-4-yl)-1H-pyrazolo[3,4-c]pyridin-3-yl)-4-methyl-2-oxopyrrolidine-3-carbonitrile as a white solid (46 mg, 45%). LCMS m/z=376.1 [M+H]⁺; ¹H NMR (500 MHz, DMSO-d₆) δ: 9.22 (s, 1H), 8.49 (s, 1H), 8.38 (s, 1H), 8.17 (d, 1H), 8.00 (s, 1H), 4.27-4.21 (m, 3H), 3.87-3.84 (m, 1H), 3.27-3.23 (m, 1H), 1.51-1.45 (m, 4H), 1.32 (d, 3H), 0.75-0.54 (m, 4H).

### Example 278

### (3R,4S)-3-cyclopropyl-4-methyl-1-(1-(1-(oxetan-3-yl)-1H-pyrazol-4-yl)-1H-pyrazolo[3,4-c]pyridin-3-yl)-2-oxopyrrolidine-3-carbonitrile

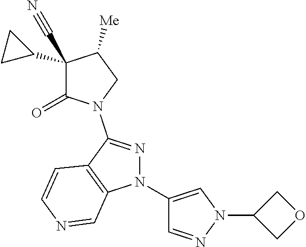 [SMILES: C[C@@H]1CN(c2nn(-c3cnn(C4COC4)c3)c3cnccc23)C(=O)[C@]1(C#N)C1CC1]

To a solution of 3-bromo-1-(1-(oxetan-3-yl)-1H-pyrazol-4-yl)-1H-pyrazolo[3,4-c]pyridine (Preparation 186, 45 mg, 0.129 mmol), (3R,4S)-3-cyclopropyl-4-methyl-2-oxopyrrolidine-3-carbonitrile (Preparation 21, 31.9 mg, 0.194 mmol), CuI (5.0 mg, 0.026 mmol) and N,N′-dimethylethane-1,2-diamine (2.3 mg, 0.026 mmol) in dioxane (10 mL) was added K₂CO₃ (53.6 mg, 0.388 mmol) at 25° C. under N₂ and stirred at 110° C. under N₂. The reaction was quenched with water (50 mL) and extracted with EtOAc (3×20 mL). The combined organics were washed with brine (20 mL), dried (Na₂SO₄) and evaporated to dryness in vacuo. The residue was purified by prep-HPLC-3 (gradient: 25-55%) to afford (3R,4S)-3-cyclopropyl-4-methyl-1-(1-(1-(oxetan-3-yl)-1H-pyrazol-4-yl)-1H-pyrazolo[3,4-c]pyridin-3-yl)-2-oxopyrrolidine-3-carbonitrile as a white solid (35 mg, 63%). LCMS m/z=404.2 [M+H]⁺; ¹H NMR (500 MHz, CDCl₃) δ: 9.21 (s, 1H), 8.62 (s, 1H), 8.33 (d, 1H), 8.12 (d, 2H), 5.67-5.59 (m, 1H), 4.96-4.94 (m, 4H), 4.22-4.16 (m, 1H), 3.83-3.77 (m, 1H), 3.25-3.16 (m, 1H), 1.47-1.44 (m, 1H), 1.27 (d, 3H), 0.71-0.50 (m, 4H).

### Example 279

### (3R,4S)-3-cyclopropyl-4-methyl-1-(1-(1-methyl-6-oxo-1,6-dihydropyridin-2-yl)-1H-pyrazolo[3,4-c]pyridin-3-yl)-2-oxopyrrolidine-3-carbonitrile

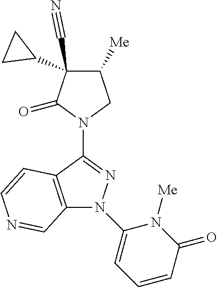 [SMILES: C[C@@H]1CN(c2nn(-c3cccc(=O)n3C)c3cnccc23)C(=O)[C@]1(C#N)C1CC1]

To a solution of 6-bromo-1-methylpyridin-2(1H)-one (150 mg, 0.800 □mol) and (3R,4S)-3-cyclopropyl-4-methyl-2-oxo-1-(1H-pyrazolo[3,4-c]pyridin-3-yl)pyrrolidine-3-carbonitrile (Preparation 189, 150 mg, 0.533 □mol) in dioxane (6 mL) was added into Cs₂CO₃ (521.2 mg, 1.60 mmol) and Cu(acac)₂ (41.9 mg, 0.160 mol) at 20° C. The reaction mixture was stirred at 100° C. under N₂ for 12 h. The reaction mixture was evaporated to dryness in vacuo and the residue purified by prep-HPLC-16 (gradient: 19-39%) to give (3R,4S)-3-cyclopropyl-4-methyl-1-(1-(1-methyl-6-oxo-1,6-dihydropyridin-2-yl)-1H-pyrazolo[3,4-c]pyridin-3-yl)-2-oxopyrrolidine-3-carbonitrile as a grey solid (50 mg, 24%). LCMS m/z=389.1 [M+H]⁺; ¹H NMR (500 MHz, DMSO-d₆) δ: 9.05 (s, 1H), 8.45 (s, 1H), 8.28 (d, 1H), 7.61 (dd, 1H), 6.68-6.60 (m, 2H), 4.26-4.22 (m, 1H), 3.84 (t, 1H), 3.27-3.20 (m, 4H), 1.52-1.49 (m, 1H), 1.31 (d, 3H), 0.76-0.74 (m, 1H), 0.68-0.65 (m, 1H), 0.59-0.55 (m, 2H).

### Example 280

### (3R,4S)-3-cyclopropyl-1-(1-(1-isopropyl-1H-pyrazol-4-yl)-1H-pyrazolo[3,4-c]pyridin-3-yl)-4-methyl-2-oxopyrrolidine-3-carbonitrile

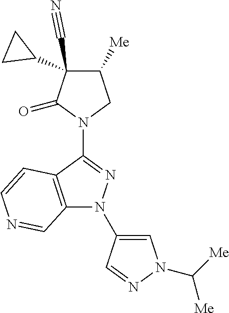 [SMILES: CC(C)n1cc(-n2nc(N3C[C@@H](C)[C@@](C#N)(C4CC4)C3=O)c3ccncc32)cn1]

To a solution of (3R,4S)-3-cyclopropyl-4-methyl-2-oxo-1-(1H-pyrazolo[3,4-c]pyridin-3-yl)pyrrolidine-3-carbonitrile hydrochloride (Preparation 189, 20 mg, 0.071 mmol) in DMF (1 mL) was added 1-isopropyl-4-(4,4,5,5-tetramethyl-1,3,2-dioxaborolan-2-yl)-1H-pyrazole (33.6 mg, 0.142 mmol), Cu(OAc)₂ (6.5 mg, 0.036 mmol), 2,2′-bipyridine (2.2 mg, 0.014 mmol) and Cs₂CO₃ (69.5 mg, 0.213 mmol) and the resulting mixture stirred at 80° C. for 2 h. The mixture was purified by prep-HPLC-2 (gradient: 38-68%) to give (3R,4S)-3-cyclopropyl-1-(1-(1-isopropyl-1H-pyrazol-4-yl)-1H-pyrazolo[3,4-c]pyridin-3-yl)-4-methyl-2-oxopyrrolidine-3-carbonitrile as a white solid (12 mg, 43%). The reaction was carried out an additional 4-times to afford a total of 56 mg of (3R,4S)-3-cyclopropyl-1-(1-(1-isopropyl-1H-pyrazol-4-yl)-1H-pyrazolo[3,4-c]pyridin-3-yl)-4-methyl-2-oxopyrrolidine-3-carbonitrile. LCMS m/z=390.2 [M+H]⁺; ¹H NMR (500 MHz, MeOD-d₄) δ: 9.09 (br s, 1H), 8.33-8.31 (m, 3H), 7.97 (s, 1H), 4.68-4.66 (m, 1H), 4.36-4.31 (m, 1H), 3.91-3.86 (m, 1H), 3.28-3.19 (m, 1H), 1.60 (d, 6H), 1.45 (d, 3H), 1.42-1.40 (m, 1H), 0.81-0.71 (m, 4H).

### Example 281-282

The title compounds were prepared from (3R,4S)-3-cyclopropyl-4-methyl-2-oxo-1-(1H-pyrazolo[3,4-c]pyridin-3-yl)pyrrolidine-3-carbonitrile hydrochloride (Preparation 189) and appropriate boronic acid/ester (RBY) using an analogous method to that described for Example 280

| Example | |
|---|---|
| No | Name/Structure/RBr/RBY/Data |
| 281 | (3R,4S)-3-cyclopropyl-4-methyl-1-(1-(2-methylpyridin-4-yl)-1H- |
| | pyrazolo[3,4-c]pyridin-3-yl)-2-oxopyrrolidine-3-carbonitrile |
| | 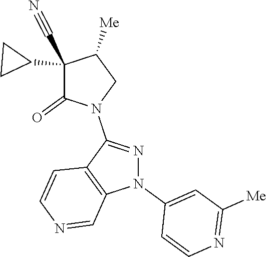 [SMILES: Cc1cc(-n2nc(N3C[C@@H](C)[C@@](C#N)(C4CC4)C3=O)c3ccncc32)ccn1] |
| | RBY: (2-methylpyridin-4-yl)boronic acid |
| | Purification: Boston Prime C18 150 * 30 mm * 5 μm; Condition: water (0.04% |
| | CH₃COOH + 10 mM CH₃COONH₄)-ACN; Begin B: 36; End B: 66; |
| | Gradient Time (min): 10; 100% B Hold Time (min): 2; Flow Rate |
| | (ml/min): 25 |
| | Yield: White solid (26 mg, 32%). |
| | LCMS m/z = 373.1 [M + H]⁺; ¹H NMR (400 MHz, MeOD-d₄) δ: 9.45 (s, |
| | 1H), 8.56 (d, 1H), 8.42-8.38 (m, 2H), 7.83 (s, 1 H), 7.80-7.77 (m, 1H), |
| | 4.39-4.34 (m, 1H), 3.91 (t, 1H), 3.24-3.18 (m, 1H), 2.65 (s, 3H), 1.42 (d, |
| | 3H), 0.79-0.76 (m, 1H), 0.72-0.69 (m, 4H). |
| 282 | (3R,4S)-3-cyclopropyl-4-methyl-1-(1-(6-methylpyridin-3-yl)-1H- |
| | pyrazolo[3,4-c]pyridin-3-yl)-2-oxopyrrolidine-3-carbonitrile |
| | 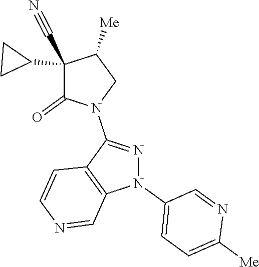 [SMILES: Cc1ccc(-n2nc(N3C[C@@H](C)[C@@](C#N)(C4CC4)C3=O)c3ccncc32)cn1] |
| | RBY: (6-methylpyridin-3-yl)boronic acid |
| | Purification: Boston Prime C18 150 * 30 mm * 5 μm; Condition: water (0.04% |
| | CH₃COOH + 10 mM CH₃COONH₄)-ACN; Begin B: 36; End B: 66; |
| | Gradient Time (min): 10; 100% B Hold Time (min): 2; Flow Rate |
| | (ml/min): 25 |
| | Yield: White solid (36 mg, 56%). |
| | LCMS m/z = 373.1 [M + H]⁺; ¹H NMR (500 MHz, MeOD-d₄) δ: 9.33 (br s, |
| | 1H), 8.92 (s, 1H), 8.43 (br s, 2H), 8.23-8.20 (m, 1H), 7.58 (d, 1H), 4.40- |
| | 4.36 (m, 1H), 3.96-3.90 (m, 1H), 3.28-3.22 (m, 1H), 2.68 (s, 3H), 1.46 (d, |
| | 3H), 1.43-1.41 (m, 1H), 0.85-0.73 (m, 4H). |

### Example 283

### (3R,4 S)-3-cyclopropyl-4-methyl-1l-(3-(1-methyl-1H-pyrazol-4-yl)pyrrolo[1,2-a]pyrimidin-6-yl)-2-oxopyrrolidine-3-carbonitrile

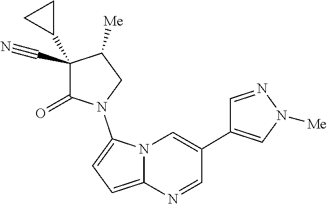 [SMILES: C[C@@H]1CN(c2ccc3ncc(-c4cnn(C)c4)cn23)C(=O)[C@]1(C#N)C1CC1]

To a solution of (3R,4S)-1-(3-bromopyrrolo[1,2-a]pyrimidin-6-yl)-3-cyclopropyl-4-methyl-2-oxopyrrolidine-3-carbonitrile (Preparation 193, 120 mg, 0.334 mmol) and 1-methyl-4-(4,4,5,5-tetramethyl-1,3,2-dioxaborolan-2-yl)-1H-pyrazole (139 mg, 0.668 mmol) in dioxane (10 mL) and H₂O (1 mL) was added K₂CO₃ (139 mg, 1.00 mmol) and Pd(dppf)Cl₂ (24.4 mg, 0.033 mmol) at 25° C. and the resulting mixture stirred at 70° C. for 3 h under N₂. The reaction mixture was evaporated to dryness in vacuo and the residue was purified by prep-HPLC-3 (Gradient: 27-47%) to afford (3R,4S)-3-cyclopropyl-4-methyl-1-(3-(1-methyl-1H-pyrazol-4-yl)pyrrolo[1,2-a]pyrimidin-6-yl)-2-oxopyrrolidine-3-carbonitrile as a yellow solid (61 mg, 51%). LCMS m/z=361.2 [M+H]⁺; ¹H NMR (400 MHz, CDCl₃) δ: 8.29 (d, 1H), 7.85 (s, 1H), 7.72 (s, 1H), 7.64 (s, 1H), 6.78 (d, 1H), 6.66 (d, 1H), 3.98 (s, 3H), 3.92-3.87 (m, 1H), 3.63-3.58 (m, 1H), 3.21-3.15 (m, 1H), 1.43 (d, 3H), 1.22-1.20 (m, 1H), 0.86-0.79 (m, 4H).

### Example 284

### (3R,4S)-3-cyclopropyl-4-methyl-1-(6-(1-methyl-1H-pyrazol-4-yl)thiazolo[5,4-c]pyridin-4-yl)-2-oxopyrrolidine-3-carbonitrile

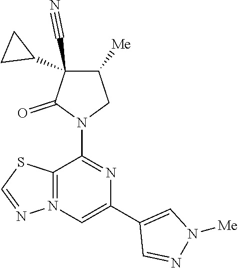

To a solution of 4-chloro-6-(1-methyl-1H-pyrazol-4-yl)thiazolo[5,4-c]pyridine (Preparation 199, 55 mg, 0.219 mmol) and (3R,4S)-3-cyclopropyl-4-methyl-2-oxopyrrolidine-3-carbonitrile (Preparation 21, 108 mg, 0.658 mmol) in dioxane (5 mL) was added K₂CO₃ (91.0 mg, 0.658 mmol), RuPhos Pd G₃ (18.4 mg, 0.022 mmol) at 25° C. under N₂ and the mixture stirred at 100° C. for 2 h under microwave irradiation. The mixture was concentrated under reduced pressure to give a residue which was purified by prep-HPLC-3 (Gradient: 33-63%) to give (3R,4S)-3-cyclopropyl-4-methyl-1-(6-(1-methyl-1H-pyrazol-4-yl)thiazolo[5,4-c]pyridin-4-yl)-2-oxopyrrolidine-3-carbonitrile as a white solid (54.2 mg, 65%). LCMS m/z=379.1 [M+H]⁺; ¹H NMR (400 MHz, CDCl₃) δ: 9.16 (s, 1H), 8.01 (d, 2H), 7.92 (s, 1H), 4.37-4.32 (s, 1H), 3.99-3.96 (m, 4H), 3.13-3.10 (m, 1H), 1.45 (d, 3H), 1.21-1.19 (m, 1H), 0.83-0.79 (m, 4H).

### Example 285

### (3R,4S)-3-cyclopropyl-4-methyl-1-(7-(1-methyl-1H-pyrazol-4-yl)-[1,2,4]triazolo[1,5-a]pyridin-5-yl)-2-oxopyrrolidine-3-carbonitrile

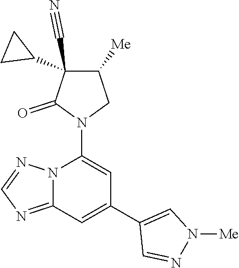 [SMILES: C[C@@H]1CN(c2cc(-c3cnn(C)c3)cc3ncnn23)C(=O)[C@]1(C#N)C1CC1]

To a solution of (3R,4S)-1-(7-chloro-[1,2,4]triazolo[1,5-a]pyridin-5-yl)-3-cyclopropyl-4-methyl-2-oxopyrrolidine-3-carbonitrile (Preparation 203, 150 mg, 0.475 mmol) and 1-methyl-4-(4,4,5,5-tetramethyl-1,3,2-dioxaborolan-2-yl)-1H-pyrazole (148 mg, 0.713 mmol) in dioxane (3 mL) and water (0.3 mL) was added KF (82.8 mg, 1.43 mmol) and Pd(dppf)Cl₂ (34.8 mg, 0.048 mmol). The mixture was stirred at 70° C. for 2 h. The mixture was concentrated and purified by prep-HPLC-3 (Gradient: 24-42%) to afford (3R,4S)-3-cyclopropyl-4-methyl-1-(7-(1-methyl-1H-pyrazol-4-yl)-[1,2,4]triazolo[1,5-a]pyridin-5-yl)-2-oxopyrrolidine-3-carbonitrile as a white solid (45 mg, 26%). LCMS m/z=362.1 [M+H]⁺; ¹H NMR (400 MHz, CDCl₃) δ: 8.27 (s, 1H), 7.84 (s, 1H), 7.77 (s, 1H), 7.73 (s, 1H), 7.31 (s, 1H), 4.22-4.16 (m, 1H), 4.04-4.02 (m, 1H), 3.96 (s, 3H), 3.17-3.15 (m, 1H), 1.44 (d, 3H), 1.30-1.29 (m, 1H), 0.92-0.90 (m, 1H), 0.80-0.78 (m, 3H).

### Example 286

### (3R,4S)-3-cyclopropyl-4-methyl-1-(6-(1-methyl-1H-pyrazol-4-yl)thieno[3,2-d]pyrimidin-4-yl)-2-oxopyrrolidine-3-carbonitrile

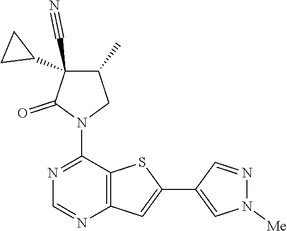 [SMILES: C[C@@H]1CN(c2ncnc3cc(-c4cnn(C)c4)sc23)C(=O)[C@]1(C#N)C1CC1]

To a solution of 4-chloro-6-(1-methyl-1H-pyrazol-4-yl)thieno[3,2-d]pyrimidine (Preparation 204, 78.6 mg, 0.479 mmol) in DMF (5 mL) was added NaH (16 mg, 60% purity) and stirred for 5 mins. (3R,4S)-3-cyclopropyl-4-methyl-2-oxopyrrolidine-3-carbonitrile (Preparation 21, 100 mg, 0.399 mol) was added to the mixture and stirred for another 10 min at 25° C. The reaction mixture was diluted with water (50 mL) and extracted with EtOAc (3×20 mL). The combined organics were washed with brine (20 mL), dried (Na₂SO₄) and evaporated to dryness in vacuo. The residue was purified by prep-HPLC-3 (Gradient: 33-63%) to afford (3R,4S)-3-cyclopropyl-4-methyl-1-(6-(1-methyl-1H-pyrazol-4-yl)thieno[3,2-d]pyrimidin-4-yl)-2-oxopyrrolidine-3-carbonitrile as a white solid (35 mg, 23%). LCMS m/z=379.1 [M+H]⁺; ¹H NMR (500 MHz, DMSO-d₆) δ: 8.87 (s, 1H), 8.46 (s, 1H), 8.08 (s, 1H), 7.68 (s, 1H), 4.32-4.27 (m, 1H), 3.90 (s, 3H), 3.88-3.85 (m, 1H), 3.16-3.12 (m, 1H), 1.52-1.49 (m, 1H), 1.29 (d, 3H), 0.75-0.73 (m, 1H), 0.67-0.66 (m, 1H), 0.59-0.54 (m, 2H).

### Example 287

### (3R,4S)-3-cyclopropyl-4-methyl-1-(7-(1-methyl-1H-pyrazol-4-yl)imidazo[1,2-a]pyridin-5-yl)-2-oxopyrrolidine-3-carbonitrile

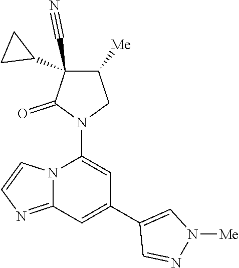 [SMILES: C[C@@H]1CN(c2cc(-c3cnn(C)c3)cc3nccn23)C(=O)[C@]1(C#N)C1CC1]

To a solution of 5-chloro-7-(1-methyl-1H-pyrazol-4-yl)imidazo[1,2-a]pyridine (Preparation 207, 70 mg, 0.301 mmol), (3R,4S)-3-cyclopropyl-4-methyl-2-oxopyrrolidine-3-carbonitrile (Preparation 21, 59.3 mg, 0.361 mmol) and CsF (91.4 mg, 0.602 mmol) in DMSO (2 mL) was added t-BuBrettphos Pd G₃ (25.8 mg, 0.030 mmol) at 25° C. and the mixture stirred for 16 h at 100° C. under N₂. The mixture was purified by prep-HPLC-3 (Gradient: 24-54%) to give (3R,4S)-3-cyclopropyl-4-methyl-1-(7-(1-methyl-1H-pyrazol-4-yl)imidazo[1,2-a]pyridin-5-yl)-2-oxopyrrolidine-3-carbonitrile as a white solid (8.0 mg, 7.4%). LCMS m/z=361.1 [M+H]⁺; ¹H NMR (500 MHz, CDCl₃) δ:7.94 (s, 1H), 7.90 (s, 1H), 7.84 (s, 1H), 7.66 (s, 1H), 7.36 (s, 1H), 7.21 (s, 1H), 4.10-4.06 (m, 1H), 3.97 (s, 3H), 3.84-3.79 (m, 1H), 3.31-3.24 (m, 1H), 1.47 (d, 3H), 1.30-1.28 (m, 1H), 0.90-0.83 (m, 4H).

### Example 288

### (3R,4S)-3-cyclopropyl-4-methyl-1-(5-(1-methyl-1H-pyrazol-4-yl)-1H-pyrrolo[2,3-b]pyridin-3-yl)-2-oxopyrrolidine-3-carbonitrile

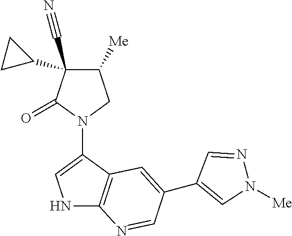 [SMILES: C[C@@H]1CN(c2c[nH]c3ncc(-c4cnn(C)c4)cc23)C(=O)[C@]1(C#N)C1CC1]

To a solution of (3R,4S)-1-(5-bromo-1H-pyrrolo[2,3-b]pyridin-3-yl)-3-cyclopropyl-4-methyl-2-oxopyrrolidine-3-carbonitrile (Preparation 195, 50 mg, 0.139 mmol) in dioxane (5 mL) and water (0.5 mL) was added 1-methyl-4-(4,4,5,5-tetramethyl-1,3,2-dioxaborolan-2-yl)-1H-pyrazole (57.9 mg, 0.278 mmol), KF (24.3 mg, 0.418 mmol) and Pd(dppf)Cl₂ (10.2 mg, 0.014 mmol) under N₂ and the resulting mixture stirred at 80° C. for 3 h. The mixture was concentrated and purified by prep-HPLC-2 (gradient: 30-60%) to afford (3R,4S)-3-cyclopropyl-4-methyl-1-(5-(1-methyl-1H-pyrazol-4-yl)-1H-pyrrolo[2,3-b]pyridin-3-yl)-2-oxopyrrolidine-3-carbonitrile as a white solid (15 mg, 30%). LCMS m/z=361.2 [M+H]⁺; ¹H NMR (500 MHz, DMSO-d₆) δ: 11.78 (s, 1H), 8.52 (s, 1H), 8.18-8.16 (m, 2H), 7.90-7.94 (m, 1H), 7.67 (s, 1H), 4.02-4.06 (m, 1H), 3.89 (s, 3H), 3.85-3.80 (m, 1H), 3.18-3.13 (m, 1H), 1.43-1.41 (m, 1H), 1.31 (d, 3H), 0.72-0.61 (m, 2H), 0.57-0.51 (m, 2H).

### Example 289

### (3R,4S)-1-(6-(1-(2-oxaspiro[3.3]heptan-6-yl)-1H-pyrazol-4-yl)pyrazolo[1,5-a]pyrazin-4-yl)-3-cyclopropyl-4-methyl-2-oxopyrrolidine-3-carbonitrile

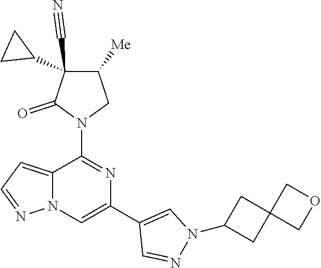 [SMILES: C[C@@H]1CN(c2nc(-c3cnn(C4CC5(COC5)C4)c3)cn3nccc23)C(=O)[C@]1(C#N)C1CC1]

N₂ was bubbled through a mixture of (3R,4S)-1-(6-chloropyrazolo[1,5-a]pyrazin-4-yl)-3-cyclopropyl-4-methyl-2-oxopyrrolidine-3-carbonitrile (Preparation 110, 100 mg, 0.317 mmol), 1-(2-oxaspiro[3.3]heptan-6-yl)-4-(4,4,5,5-tetramethyl-1,3,2-dioxaborolan-2-yl)-1H-pyrazole (Preparation 231, 100 mg, 0.345 mmol), K₂CO₃ (142.89 mg, 1.03 mmol), PEPPSI-iPr (23.49 mg, 0.0345 mmol) in dioxane (10 mL) and water (2 mL) and the reaction was stirred at 90° C. for 2 h. The reaction mixture was concentrated in vacuum to give the crude, which was purified by prep HPLC-3 (31 to 61% gradient) to give (3R,4S)-1-(6-(1-(2-oxaspiro[3.3]heptan-6-yl)-1H-pyrazol-4-yl)pyrazolo[1,5-a]pyrazin-4-yl)-3-cyclopropyl-4-methyl-2-oxopyrrolidine-3-carbonitrile (46.20 mg, 29.9% yield) as a white solid. LCMS m/z=444.1 [M+H]⁺; ¹H NMR (500 MHz, MeOD-d₄) δ=8.76 (s, 1H), 8.22 (s, 1H), 8.12-8.00 (m, 2H), 7.00 (d, J=2.5 Hz, 1H), 4.85 (s, 2H), 4.81-4.76 (m, 1H), 4.75 (s, 2H), 4.40-4.35 (m, 1H), 3.99 (t, J=10.5 Hz, 1H), 3.22-3.12 (m, 1H), 2.92-2.85 (m, 2H), 2.83-2.76 (m, 2H), 1.46-1.39 (m, 4H), 0.85-0.79 (m, 1H), 0.77-0.71 (m, 3H).

### Example 290

### (3R,4S)-3-cyclopropyl-1-(6-(1-(3,3-difluorocyclobutyl)-1H-pyrazol-4-yl)pyrazolo[1,5-a]pyrazin-4-yl)-4-methyl-2-oxopyrrolidine-3-carbonitrile

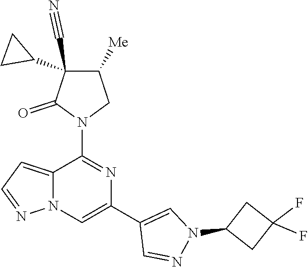

(3R,4S)-3-Cyclopropyl-1-(6-(1-(3,3-difluorocyclobutyl)-1H-pyrazol-4-yl)pyrazolo[1,5-a]pyrazin-4-yl)-4-methyl-2-oxopyrrolidine-3-carbonitrile was obtained as a white solid, 18.3 mg, 14% yield, from 1-(3,3-difluorocyclobutyl)-4-(4,4,5,5-tetramethyl-1,3,2-dioxaborolan-2-yl)-1H-pyrazole (Preparation 237) and (3R,4S)-1-(6-chloropyrazolo[1,5-a]pyrazin-4-yl)-3-cyclopropyl-4-methyl-2-oxopyrrolidine-3-carbonitrile (Preparation 110) following a similar procedure to that described in Example 289. LCMS m/z=438.1 [M+H]⁺; ¹H NMR (500 MHz, MeOD-d₄) δ=8.79 (s, 1H), 8.30 (s, 1H), 8.12 (s, 1H), 8.04 (d, J=2.5 Hz, 1H), 7.02 (d, J=2.0 Hz, 1H), 4.60 (s, 1H), 4.40-4.37 (m, 1H), 4.00 (t, J=10.5 Hz, 1H), 3.29-3.14 (m, 5H), 1.47-1.42 (m, 4H), 0.85-0.71 (m, 4H).

### Example 291

### (3R,4S)-3-cyclopropyl-1-(3-fluoro-6-(1-(oxetan-3-yl)-1H-pyrazol-4-yl)pyrazolo[1,5-a]pyrazin-4-yl)-4-methyl-2-oxopyrrolidine-3-carbonitrile

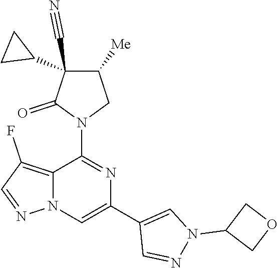 [SMILES: C[C@@H]1CN(c2nc(-c3cnn(C4COC4)c3)cn3ncc(F)c23)C(=O)[C@]1(C#N)C1CC1]

(3R,4S)-3-Cyclopropyl-1-(3-fluoro-6-(1-(oxetan-3-yl)-1H-pyrazol-4-yl)pyrazolo[1,5-a]pyrazin-4-yl)-4-methyl-2-oxopyrrolidine-3-carbonitrile was obtained as a white solid, 109.9 mg, 72.5% yield from (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125) and 1-(oxetan-3-yl)-4-(4,4,5,5-tetramethyl-1,3,2-dioxaborolan-2-yl)-1H-pyrazole following a similar procedure to that described in Example 289. LCMS m/z=422.1 [M+H]⁺; ¹H NMR (500 MHz, MeOD-d₄) δ=8.71 (s, 1H), 8.35 (s, 1H), 8.16 (s, 1H), 7.99 (d, J=3.5 Hz, 1H), 5.63 (m, 1H), 5.14-5.05 (m, 4H), 4.37 (dd, J=7.5, 10.0 Hz, 1H), 3.93 (t, J=9.5 Hz, 1H), 3.26-3.15 (m, 1H), 1.49-1.37 (m, 4H), 0.86-0.72 (m, 4H).

### Example 292 and 293

### (3R,4S)-1-(6-(1-((1s,3S)-3-cyanocyclobutyl)-1H-pyrazol-4-yl)-3-fluoropyrazolo[1,5-a]pyrazin-4-yl)-3-cyclopropyl-4-methyl-2-oxopyrrolidine-3-carbonitrile and (3R,4S)-1-(6-(1-((1r,3R)-3-cyanocyclobutyl)-1H-pyrazol-4-yl)-3-fluoropyrazolo[1,5-a]pyrazin-4-yl)-3-cyclopropyl-4-methyl-2-oxopyrrolidine-3-carbonitrile

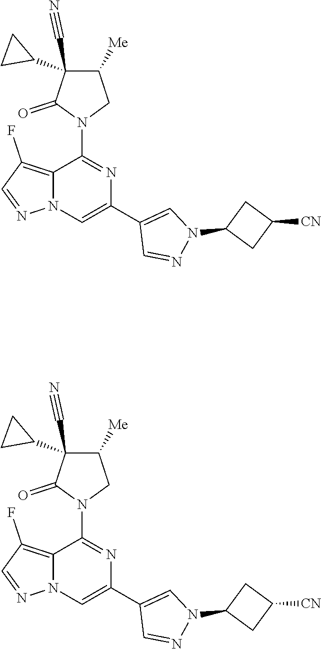 [SMILES: C[C@@H]1CN(c2nc(-c3cnn([C@H]4C[C@@H](C#N)C4)c3)cn3ncc(F)c23)C(=O)[C@]1(C#N)C1CC1 ; C[C@@H]1CN(c2nc(-c3cnn([C@H]4C[C@H](C#N)C4)c3)cn3ncc(F)c23)C(=O)[C@]1(C#N)C1CC1]

N₂ was bubbled through a mixture of 3-(4-(4,4,5,5-tetramethyl-1,3,2-dioxaborolan-2-yl)-1H-pyrazol-1-yl)cyclobutane-1-carbonitrile (Preparation 232, 300 mg, 1.10 mmol), (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 260 mg, 0.779 mmol), K₂CO₃ (323 mg, 2.34 mmol) and PEPPSI-iPr (53.09 mg, 0.0779 mmol) in dioxane (10 mL) and water (2 mL) and the reaction was stirred at 90° C. for 2 h. The reaction mixture was concentrated in vacuo to give crude product, which was purified by prep HPLC-3 (37 to 67% gradient) to give peak 1, Example 292: (3R,4S)-1-(6-(1-((1s,3S)-3-cyanocyclobutyl)-1H-pyrazol-4-yl)-3-fluoropyrazolo[1,5-a]pyrazin-4-yl)-3-cyclopropyl-4-methyl-2-oxopyrrolidine-3-carbonitrile (86 mg, 24.8% yield) as a white solid. The relative stereochemistry was determined using 2D nmr. LCMS m/z=445.1 [M+H]⁺; ¹H NMR (500 MHz, DMSO-d₆) δ=9.01 (s, 1H), 8.40 (s, 1H), 8.23 (d, J=3.5 Hz, 1H), 8.17 (s, 1H), 5.28-5.15 (m, 1H), 4.35-4.30 (m, 1H), 3.87 (t, J=10.0 Hz, 1H), 3.56-3.48 (m, 1H), 3.21-3.13 (m, 1H), 2.95-2.87 (m, 2H), 2.85-2.78 (m, 2H), 1.48-1.42 (m, 1H), 1.33 (d, J=6.5 Hz, 3H), 0.79-0.74 (m, 1H), 0.71-0.66 (m, 1H), 0.61-0.55 (m, 2H).

And Peak 2, Example 293: (3R,4S)-1-(6-(1-((1r,3R)-3-cyanocyclobutyl)-1H-pyrazol-4-yl)-3-fluoropyrazolo[1,5-a]pyrazin-4-yl)-3-cyclopropyl-4-methyl-2-oxopyrrolidine-3-carbonitrile (25.9 mg, 7.5% yield) as a white solid. The relative stereochemistry was determined using 2D nmr. LCMS m/z=445.1 [M+H]⁺; ¹H NMR (500 MHz, DMSO-d₆) δ=9.02 (d, J=1.0 Hz, 1H), 8.45 (s, 1H), 8.23 (d, J=4.0 Hz, 1H), 8.17 (s, 1H), 4.94 (m, 1H), 4.36-4.30 (m, 1H), 3.88 (t, J=10.0 Hz, 1H), 3.30-3.22 (m, 1H), 3.20-3.13 (m, 1H), 2.91-2.78 (m, 4H), 1.49-1.41 (m, 1H), 1.33 (d, J=7.0 Hz, 3H), 0.79-0.73 (m, 1H), 0.71-0.65 (m, 1H), 0.61-0.55 (m, 2H).

### Example 294

### (3R,4S)-3-cyclopropyl-4-methyl-2-oxo-1-(6-(1-(pyridin-3-yl)-1H-pyrazol-4-yl)pyrazolo[1,5-a]pyrazin-4-yl)pyrrolidine-3-carbonitrile

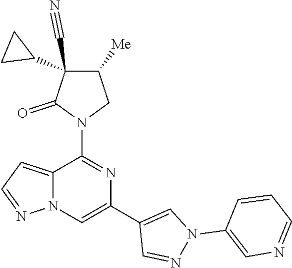 [SMILES: C[C@@H]1CN(c2nc(-c3cnn(-c4cccnc4)c3)cn3nccc23)C(=O)[C@]1(C#N)C1CC1]

To a mixture of (3R,4S)-1-(6-chloropyrazolo[1,5-a]pyrazin-4-yl)-3-cyclopropyl-4-methyl-2-oxopyrrolidine-3-carbonitrile (Preparation 110, 110 mg, 0.348 mmol), 3-(4-(4,4,5,5-tetramethyl-1,3,2-dioxaborolan-2-yl)-1H-pyrazol-1-yl)pyridine (188.9 mg, 0.697 mmol) and K₃PO₄ (221.8 mg, 1.05 mmol) in dioxane (10 mL) and water (2 mL) was added Pd(t-Bu₃P)₂ (17.80 mg, 0.0348 mmol) and the reaction was stirred at 90° C. for 2 h. The reaction mixture was concentrated to give a crude, which was purified by prep-HPLC-11 (33 to 53% gradient) to give (3R,4S)-3-cyclopropyl-4-methyl-2-oxo-1-(6-(1-(pyridin-3-yl)-1H-pyrazol-4-yl)pyrazolo[1,5-a]pyrazin-4-yl)pyrrolidine-3-carbonitrile (47.8 mg, 32.3% yield) as a grey solid. LCMS m/z=425.1 [M+H]⁺; ¹H NMR (500 MHz, DMSO-d₆) δ=9.26 (d, J=0.5 Hz, 1H), 9.20 (s, 1H), 9.18 (s, 1H), 8.58-8.57 (m, 1H), 8.50 (s, 1H), 8.33-8.31 (m, 1H), 8.18 (s, 1H), 7.62-7.59 (m, 1H), 6.98 (d, J=1.5 Hz, 1H), 4.49-4.45 (m, 1H), 4.00-3.96 (m, 1H), 3.24-3.19 (m, 1H), 1.52-1.50 (m, 1H), 1.36 (d, J=7.0 Hz, 3H), 0.76-0.71 (m, 2H), 0.61-0.58 (m, 2H).

### Example 295

### (3R,4S)-3-cyclopropyl-4-methyl-2-oxo-1-(6-(1-(pyridin-4-yl)-1H-pyrazol-4-yl)pyrazolo[1,5-a]pyrazin-4-yl)pyrrolidine-3-carbonitrile

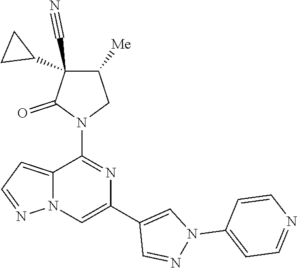 [SMILES: C[C@@H]1CN(c2nc(-c3cnn(-c4ccncc4)c3)cn3nccc23)C(=O)[C@]1(C#N)C1CC1]

(3R,4S)-3-Cyclopropyl-4-methyl-2-oxo-1-(6-(1-(pyridin-4-yl)-1H-pyrazol-4-yl)pyrazolo[1,5-a]pyrazin-4-yl)pyrrolidine-3-carbonitrile was obtained as a white solid, 66 mg, 26.4% yield, from (3R,4S)-1-(6-chloropyrazolo[1,5-a]pyrazin-4-yl)-3-cyclopropyl-4-methyl-2-oxopyrrolidine-3-carbonitrile (Preparation 110) and 4-(4-(4,4,5,5-tetramethyl-1,3,2-dioxaborolan-2-yl)-1H-pyrazol-1-yl)pyridine, following a similar procedure to that described in Example 294. LCMS m/z=425.3 [M+H]⁺; ¹HNMR (400 MHz, DMSO-d₆) δ: 9.27 (s, 1H), 9.24 (s, 1H), 8.70 (d, J=6.0 Hz, 2H), 8.52 (s, 1H), 8.18 (d, J=2.4 Hz, 1H), 7.94 (d, J=6.4 Hz, 2H), 6.97 (d, J=2.0 Hz, 1H), 4.46 (t, J=8.0 Hz, 1H), 3.97 (t, J=10.8 Hz, 1H), 3.25-3.16 (m, 1H), 1.52-1.47 (m, 1H), 1.35 (d, J=6.8 Hz, 3H), 0.77-0.67 (m, 2H), 0.62-0.56 (m, 2H).

### Example 296

### (3R,4S)-3-cyclopropyl-4-methyl-2-oxo-1-(6-(1-(pyridin-2-yl)-1H-pyrazol-4-yl)pyrazolo[1,5-a]pyrazin-4-yl)pyrrolidine-3-carbonitrile

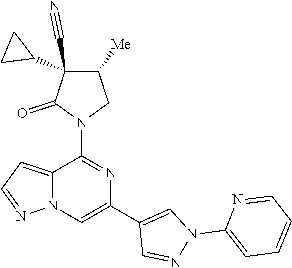 [SMILES: C[C@@H]1CN(c2nc(-c3cnn(-c4ccccn4)c3)cn3nccc23)C(=O)[C@]1(C#N)C1CC1]

To a mixture of 2-(4-(4,4,5,5-tetramethyl-1,3,2-dioxaborolan-2-yl)-1H-pyrazol-1-yl)pyridine (130 mg, 0.479 mmol), (3R,4S)-1-(6-chloropyrazolo[1,5-a]pyrazin-4-yl)-3-cyclopropyl-4-methyl-2-oxopyrrolidine-3-carbonitrile (Preparation 110, 106.0 mg, 0.336 mmol) and K₃PO₄ (305.3 mg, 1.44 mmol) in dioxane (8 mL) and water (1.6 mL) was added Pd(t-Bu₃P)₂ (24.50 mg, 0.048 mmol), and the reaction was stirred at 90° C. for 2 h. The reaction mixture was concentrated in vacuo and the crude was purified by prep-HPLC-3 (46 to 76% gradient) to give (3R,4S)-3-cyclopropyl-4-methyl-2-oxo-1-(6-(1-(pyridin-2-yl)-1H-pyrazol-4-yl)pyrazolo[1,5-a]pyrazin-4-yl)pyrrolidine-3-carbonitrile (31.0 mg, 15.2% yield) as a gray solid. LCMS m/z=425.2 [M+H]⁺; ¹H NMR (500 MHz, DMSO-d₆) δ=9.37 (s, 1H), 9.19 (s, 1H), 8.54 (d, J=4.0 Hz, 1H), 8.51 (s, 1H), 8.18 (d, J=2.5 Hz, 1H), 8.05-8.03 (m, 1H), 8.01-7.99 (m, 1H), 7.43-7.41 (m, 1H), 6.98 (d, J=1.5 Hz, 1H), 4.42-4.39 (m, 1H), 4.03-3.99 (m, 1H), 3.23-3.18 (m, 1H), 1.56-1.54 (m, 1H), 1.35 (d, J=7.0 Hz, 3H), 0.77-0.75 (m, 1H), 0.71-0.69 (m, 1H), 0.60-0.57 (m, 2H).

### Example 297

### (3R,4S)-1-(6-(1-(3-cyanobicyclo[1.1.1]pentan-1-yl)-1H-pyrazol-4-yl)-3-fluoropyrazolo[1,5-a]pyrazin-4-yl)-3-cyclopropyl-4-methyl-2-oxopyrrolidine-3-carbonitrile

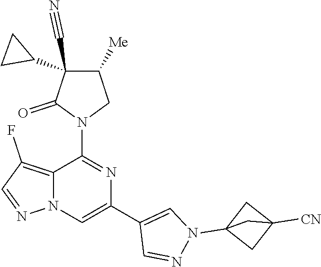 [SMILES: C[C@@H]1CN(c2nc(-c3cnn(C45CC(C#N)(C4)C5)c3)cn3ncc(F)c23)C(=O)[C@]1(C#N)C1CC1]

To a solution of (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 46.82 mg, 0.140 mmol) and 3-(4-(4,4,5,5-tetramethyl-1,3,2-dioxaborolan-2-yl)-1H-pyrazol-1-yl)bicyclo[1.1.1]pentane-1-carbonitrile (Preparation 233, 40 mg, 0.140 mmol) in a mixture of dioxane (2 mL) and water (0.3 mL) was added K₂CO₃ (38.78 mg, 0.281 mmol) and Pd(dtbpf)Cl₂ (9.14 mg, 0.014 mmol) at 15° C. and the reaction was stirred at 90° C. under N₂ for 2 h. The reaction mixture was concentrated in vacuo and the crude product was purified by prep-HPLC-16 (51 to 61% gradient) to give (3R,4S)-1-(6-(1-(3-cyanobicyclo[1.1.1]pentan-1-yl)-1H-pyrazol-4-yl)-3-fluoropyrazolo[1,5-a]pyrazin-4-yl)-3-cyclopropyl-4-methyl-2-oxopyrrolidine-3-carbonitrile (7 mg, 10.9% yield) as a white solid. LCMS m/z=457.2 [M+H]⁺; ¹H NMR (400 MHz, MeOD-d₄) δ=8.71 (s, 1H), 8.29 (s, 1H), 8.12 (s, 1H), 8.00 (s, 1H), 4.40-4.34 (m, 1H), 3.96-3.90 (m, 1H), 3.23-3.15 (m, 1H), 2.86-2.84 (m, 6H), 1.46-1.41 (m, 4H), 0.83-0.73 (m, 4H).

### Example 298

### (3R,4S)-1-(6-(1-(2-oxaspiro[3.3]heptan-6-yl)-1H-pyrazol-4-yl)-3-fluoropyrazolo[1,5-a]pyrazin-4-yl)-3-cyclopropyl-4-methyl-2-oxopyrrolidine-3-carbonitrile

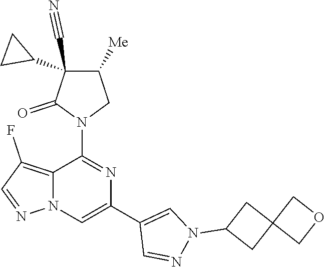 [SMILES: C[C@@H]1CN(c2nc(-c3cnn(C4CC5(COC5)C4)c3)cn3ncc(F)c23)C(=O)[C@]1(C#N)C1CC1]

N₂ was bubbled through a mixture of 1-(2-oxaspiro[3.3]heptan-6-yl)-4-(4,4,5,5-tetramethyl-1,3,2-dioxaborolan-2-yl)-1H-pyrazole (Preparation 231, 110 mg, 0.379 mmol), (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 80 mg, 0.24 mmol), K₂CO₃ (99.38 mg, 0.719 mmol) and Pd(dtbpf)Cl₂ (16.34 mg, 0.024 mmol) in dioxane (10 mL) and water (2 mL) and the reaction was stirred at 90° C. for 2 h. The reaction mixture was concentrated in vacuo and the crude product was purified by prep HPLC-21 (gradient: 45 to 72%) to give (3R,4S)-1-(6-(1-(2-oxaspiro[3.3]heptan-6-yl)-1H-pyrazol-4-yl)-3-fluoropyrazolo[1,5-a]pyrazin-4-yl)-3-cyclopropyl-4-methyl-2-oxopyrrolidine-3-carbonitrile (20.3 mg, 17.7% yield) as a white solid. LCMS m/z=462.1 [M+H]⁺; ¹H NMR (400 MHz, MeOD-d₄) δ=8.67 (d, J=1.6 Hz, 1H), 8.23 (s, 1H), 8.06 (s, 1H), 7.98 (d, J=3.6 Hz, 1H), 4.85 (s, 2H), 4.78 (d, J=8.0 Hz, 1H), 4.75 (s, 2H), 4.36 (dd, J₁=8.0 Hz, J₂=10.4 Hz, 1H), 3.93 (t, J=10.4 Hz, 1H), 3.26-3.15 (m, 1H), 2.91-2.85 (m, 2H), 2.83-2.75 (m, 2H), 1.48-1.38 (m, 4H), 0.83-0.71 (m, 4H).

### Example 299

### (3R,4S)-3-cyclopropyl-1-(3-fluoro-6-(1-((1r,3R)-3-methoxycyclobutyl)-1H-pyrazol-4-yl)pyrazolo[1,5-a]pyrazin-4-yl)-4-methyl-2-oxopyrrolidine-3-carbonitrile

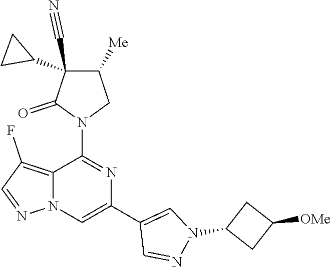 [SMILES: CO[C@H]1C[C@H](n2cc(-c3cn4ncc(F)c4c(N4C[C@@H](C)[C@@](C#N)(C5CC5)C4=O)n3)cn2)C1]

To a solution of 1-((1r,3r)-3-methoxycyclobutyl)-4-(4,4,5,5-tetramethyl-1,3,2-dioxaborolan-2-yl)-1H-pyrazole (Preparation 235, 160 mg, 0.575 mmol) in dioxane (8 mL) and water (1.6 mL) was added K₂CO₃ (238.5 mg, 1.73 mmol) and (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 192.0 mg, 0.575 mmol) at 20° C. Pd(dtbpf)Cl₂ (74.98 mg, 0.115 mmol) was added and the reaction was stirred at 90° C. under N₂ for 2 h. The mixture was poured into water (50 mL) and extracted with DCM (50 mL×3). The combined organic layers were washed with brine (100 mL), dried over Na₂SO₄, filtered and concentrated in vacuo. The crude product was purified by Prep-HPLC-3 (37 to 67% gradient) to give (3R,4S)-3-cyclopropyl-1-(3-fluoro-6-(1-((1r,3R)-3-methoxycyclobutyl)-1H-pyrazol-4-yl)pyrazolo[1,5-a]pyrazin-4-yl)-4-methyl-2-oxopyrrolidine-3-carbonitrile (64.1 mg, 22.8% yield) as a yellow solid. LCMS m/z=450.2 [M+H]⁺; ¹H NMR (400 MHz, CDCl₃) δ: =8.25 (d, J=1.2 Hz, 1H), 7.91 (s, 1H), 7.86 (s, 1H), 7.82 (d, J=4.0 Hz, 1H), 5.02-4.93 (m, 1H), 4.32-4.23 (m, 2H), 3.81 (t, J=9.6 Hz, 1H), 3.33 (s, 3H), 3.23-3.14 (m, 1H), 2.86-2.79 (m, 2H), 2.64-2.57 (m, 2H), 1.45 (d, J=7.2 Hz, 3H), 1.24-1.18 (m, 1H), 0.89-0.76 (m, 4H).

### Example 300

### (3R,4S)-3-cyclopropyl-1-(3-fluoro-6-(1-(3-methyloxetan-3-yl)-1H-pyrazol-4-yl)pyrazolo[1,5-a]pyrazin-4-yl)-4-methyl-2-oxopyrrolidine-3-carbonitrile

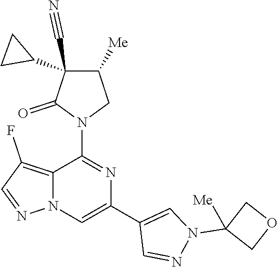 [SMILES: C[C@@H]1CN(c2nc(-c3cnn(C4(C)COC4)c3)cn3ncc(F)c23)C(=O)[C@]1(C#N)C1CC1]

(3R,4S)-3-Cyclopropyl-1-(3-fluoro-6-(1-(3-methyloxetan-3-yl)-1H-pyrazol-4-yl)pyrazolo[1,5-a]pyrazin-4-yl)-4-methyl-2-oxopyrrolidine-3-carbonitrile was obtained as a yellow solid, 11 mg, 16.7% yield, from 1-(3-methyloxetan-3-yl)-4-(4,4,5,5-tetramethyl-1,3,2-dioxaborolan-2-yl)-1H-pyrazole (Preparation 234) and (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125) following the procedure described in Example 299. LCMS m/z=436.3 [M+H]^{+ 1}H NMR (400 MHz, CDCl₃) δ=8.29 (d, J=1.2 Hz, 1H), 7.97 (d, J=6.4 Hz, 2H), 7.84 (d, J=3.6 Hz, 1H), 5.22 (d, J=6.4 Hz, 2H), 4.72 (d, J=6.8 Hz, 2H), 4.35-4.28 (m, 1H), 3.82 (t, J=9.6 Hz, 1H), 3.23-3.13 (m, 1H), 2.03 (s, 3H), 1.46 (d, J=7.2 Hz, 3H), 1.25-1.18 (m, 1H), 0.90-0.75 (m, 4H).

### Example 301

### (3R,4S)-3-cyclopropyl-1-(3-fluoro-6-(1-((1s,3 S)-3-hydroxy-3-methylcyclobutyl)-1H-pyrazol-4-yl)pyrazolo[1,5-a]pyrazin-4-yl)-4-methyl-2-oxopyrrolidine-3-carbonitrile

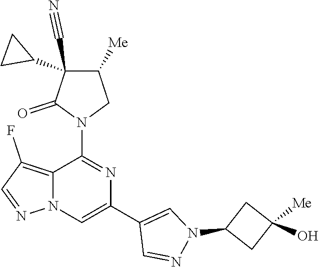 [SMILES: C[C@@H]1CN(c2nc(-c3cnn([C@H]4C[C@@](C)(O)C4)c3)cn3ncc(F)c23)C(=O)[C@]1(C#N)C1CC1]

(3R,4S)-3-Cyclopropyl-1-(3-fluoro-6-(1-((1s,3 S)-3-hydroxy-3-methylcyclobutyl)-1H-pyrazol-4-yl)pyrazolo[1,5-a]pyrazin-4-yl)-4-methyl-2-oxopyrrolidine-3-carbonitrile was obtained as a yellow solid, 21.2 mg, 10.8% yield, from (1s,3s)-1-methyl-3-(4-(4,4,5,5-tetramethyl-1,3,2-dioxaborolan-2-yl)-1H-pyrazol-1-yl)cyclobutan-1-ol (Preparation 241) and (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125) following the procedure described in Example 299. LCMS m/z=450.2 [M+H]⁺; ¹H NMR (400 MHz, CDCl₃) δ=8.25 (s, 1H), 7.91 (d, J=6.8 Hz, 2H), 7.83 (d, J=3.6 Hz, 1H), 4.60-4.50 (m, 1H), 4.33-4.26 (m, 1H), 3.82 (t, J=9.2 Hz, 1H), 3.62-3.24 (m, 1H), 3.24-3.12 (m, 1H), 2.81-2.74 (m, 2H), 2.71-2.63 (m, 2H), 1.49 (s, 3H), 1.45 (d, J=6.8 Hz, 3H), 1.25-1.17 (m, 1H), 0.89-0.76 (m, 4H).

### Example 302

### (3R,4S)-1-(6-(1-((1-cyanocyclopropyl)methyl)-1H-pyrazol-4-yl)-3-fluoropyrazolo[1,5-a]pyrazin-4-yl)-3-cyclopropyl-4-methyl-2-oxopyrrolidine-3-carbonitrile

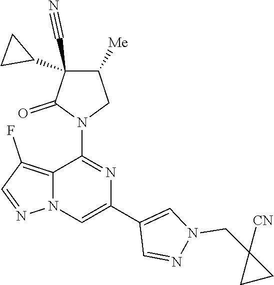 [SMILES: C[C@@H]1CN(c2nc(-c3cnn(CC4(C#N)CC4)c3)cn3ncc(F)c23)C(=O)[C@]1(C#N)C1CC1]

(3R,4S)-1-(6-(1-((1-Cyanocyclopropyl)methyl)-1H-pyrazol-4-yl)-3-fluoropyrazolo[1,5-a]pyrazin-4-yl)-3-cyclopropyl-4-methyl-2-oxopyrrolidine-3-carbonitrile was obtained as a yellow solid, 34.5 mg, 21.2% yield, from 1-((4-(4,4,5,5-tetramethyl-1,3,2-dioxaborolan-2-yl)-1H-pyrazol-1-yl)methyl)cyclopropane-1-carbonitrile (Preparation 242) and (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125) following a similar procedure to that described in Example 299. LCMS m/z=445.2 [M+H]⁺; ¹H NMR (400 MHz, CDCl₃) δ=8.29 (d, J=1.2 Hz, 1H), 8.03 (s, 1H), 7.92 (s, 1H), 7.84 (d, J=4.0 Hz, 1H), 4.33-4.29 (m, 1H), 4.29-4.22 (m, 2H), 3.84 (t, J=10.0 Hz, 1H), 3.27-3.10 (m, 1H), 1.47-1.42 (m, 5H), 1.33-1.27 (m, 2H), 1.26-1.21 (m, 1H), 0.90-0.76 (m, 4H).

### Example 303

### (3R,4S)-3-cyclopropyl-1-(3-fluoro-6-(1-((3-methyloxetan-3-yl)methyl)-1H-pyrazol-4-yl)pyrazolo[1,5-a]pyrazin-4-yl)-4-methyl-2-oxopyrrolidine-3-carbonitrile

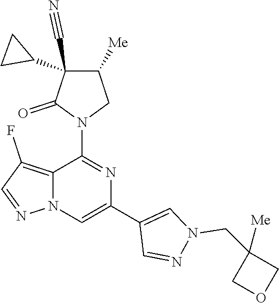 [SMILES: C[C@@H]1CN(c2nc(-c3cnn(CC4(C)COC4)c3)cn3ncc(F)c23)C(=O)[C@]1(C#N)C1CC1]

(3R,4S)-3-Cyclopropyl-1-(3-fluoro-6-(1-((3-methyloxetan-3-yl)methyl)-1H-pyrazol-4-yl)pyrazolo[1,5-a]pyrazin-4-yl)-4-methyl-2-oxopyrrolidine-3-carbonitrile was obtained as a yellow solid, 35.5 mg, 22.0% yield, from 1-((3-methyloxetan-3-yl)methyl)-4-(4,4,5,5-tetramethyl-1,3,2-dioxaborolan-2-yl)-1H-pyrazole (Preparation 243) and (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125) following the procedure described in Example 299. LCMS m/z=450.2 [M+H]⁺; ¹H NMR (400 MHz, CDCl₃) δ=8.26 (d, J=1.2 Hz, 1H), 7.88 (s, 1H), 7.86-7.78 (m, 2H), 4.74 (d, J=6.4 Hz, 2H), 4.48-4.43 (m, 2H), 4.39 (s, 2H), 4.34-4.27 (m, 1H), 3.81 (t, J=10.0 Hz, 1H), 3.24-3.12 (m, 1H), 1.45 (d, J=6.8 Hz, 3H), 1.31 (s, 3H), 1.26-1.17 (m, 1H), 0.90-0.76 (m, 4H).

### Example 304

### (3R,4S)-3-cyclopropyl-1-(3-fluoro-6-(1-(oxetan-3-ylmethyl)-1H-pyrazol-4-yl)pyrazolo[1,5-a]pyrazin-4-yl)-4-methyl-2-oxopyrrolidine-3-carbonitrile

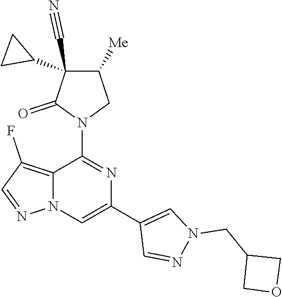 [SMILES: C[C@@H]1CN(c2nc(-c3cnn(CC4COC4)c3)cn3ncc(F)c23)C(=O)[C@]1(C#N)C1CC1]

To a solution of (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 100 mg, 0.30 mmol) in dioxane (5 mL) and water (1 mL) was added 1-(oxetan-3-ylmethyl)-4-(4,4,5,5-tetramethyl-1,3,2-dioxaborolan-2-yl)-1H-pyrazole (158.28 mg, 0.599 mmol) and K₂CO₃ (124.23 mg, 0.899 mmol) at 20° C. Pd(dtbpf)Cl₂ (39.06 mg, 0.06 mmol) was added and the reaction was stirred at 90° C. under N₂ for 2 h. The reaction was quenched with water (30 mL), extracted with DCM (20 mL×3) and the combined organic extracts concentrated in vacuo. The residue was purified by Prep-HPLC-2 (44 to 64% gradient) to give (3R,4S)-3-cyclopropyl-1-(3-fluoro-6-(1-(oxetan-3-ylmethyl)-1H-pyrazol-4-yl)pyrazolo[1,5-a]pyrazin-4-yl)-4-methyl-2-oxopyrrolidine-3-carbonitrile (32.9 mg, 25.2% yield) as a yellow solid. LCMS m/z=436.3 [M+H]⁺; 1H NMR (400 MHz, CDCl₃) δ=8.18 (s, 1H), 7.80 (s, 1H), 7.75-7.70 (m, 2H), 4.81-4.78 (m, 2H), 4.50-4.46 (m, 2H), 4.43-4.41 (m, 2H), 4.24-4.20 (m, 1H), 3.75-3.70 (m, 1H), 3.55-3.45 (m, 1H), 3.20-3.05 (m, 1H), 1.38 (d, J=6.8 Hz, 3H), 1.18-1.10 (m, 1H), 0.80-0.65 (m, 4H).

### Example 305

### (3R,4S)-3-cyclopropyl-1-(3-fluoro-6-(1-((2S,3S)-2-methyloxetan-3-yl)-1H-pyrazol-4-yl)pyrazolo[1,5-a]pyrazin-4-yl)-4-methyl-2-oxopyrrolidine-3-carbonitrile and (3R,4S)-3-cyclopropyl-1-(3-fluoro-6-(1-((2R,3R)-2-methyloxetan-3-yl)-1H-pyrazol-4-yl)pyrazolo[1,5-a]pyrazin-4-yl)-4-methyl-2-oxopyrrolidine-3-carbonitrile

### and Example 306

### (3R,4S)-3-cyclopropyl-1-(3-fluoro-6-(1-((2R,3S)-2-methyloxetan-3-yl)-1H-pyrazol-4-yl)pyrazolo[1,5-a]pyrazin-4-yl)-4-methyl-2-oxopyrrolidine-3-carbonitrile and (3R,4S)-3-cyclopropyl-1-(3-fluoro-6-(1-((2S,3R)-2-methyloxetan-3-yl)-1H-pyrazol-4-yl)pyrazolo[1,5-a]pyrazin-4-yl)-4-methyl-2-oxopyrrolidine-3-carbonitrile

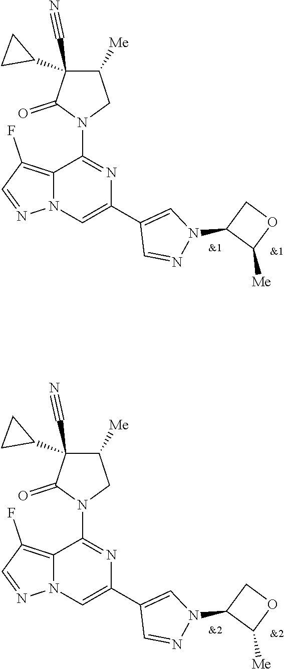 [SMILES: C[C@@H]1OC[C@@H]1n1cc(-c2cn3ncc(F)c3c(N3C[C@@H](C)[C@@](C#N)(C4CC4)C3=O)n2)cn1 ; C[C@@H]1CN(c2nc(-c3cnn([C@H]4CO[C@@H]4C)c3)cn3ncc(F)c23)C(=O)[C@]1(C#N)C1CC1]

To a mixture of 1-(2-methyloxetan-3-yl)-4-(4,4,5,5-tetramethyl-1,3,2-dioxaborolan-2-yl)-1H-pyrazole (Preparation 238, 50 mg, 0.150 mmol), (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 47.48 mg, 0.180 mmol) and K₂CO₃ (62.12 mg, 0.449 mmol) in dioxane (1.5 mL) and H₂O (0.3 mL) was added Pd(dtbpf)Cl₂ (9.76 mg, 0.015 mmol) and the reaction was stirred at 90° C. for 2 h. The reaction mixture was concentrated to give a crude, which was purified by prep-HPLC-3 (30 to 59% gradient) give peak 1, Example 305 (3R,4S)-3-cyclopropyl-1-(3-fluoro-6-(1-((2S,3S)-2-methyloxetan-3-yl)-1H-pyrazol-4-yl)pyrazolo[1,5-a]pyrazin-4-yl)-4-methyl-2-oxopyrrolidine-3-carbonitrile and (3R,4S)-3-cyclopropyl-1-(3-fluoro-6-(1-((2R,3R)-2-methyloxetan-3-yl)-1H-pyrazol-4-yl)pyrazolo[1,5-a]pyrazin-4-yl)-4-methyl-2-oxopyrrolidine-3-carbonitrile (9.2 mg, 14.10% yield) as a white solid. The relative stereochemistry was determined by 2D nmr. LCMS m/z=436.1 [M+H]⁺; ¹H NMR (500 MHz, MeOD-d₄) δ=8.73 (s, 1H), 8.39 (s, 1H), 8.16 (s, 1H), 7.99 (d, J=3.5 Hz, 1H), 5.56-5.53 (m, 1H), 5.36-5.33 (m, 1H), 5.24-5.23 (m, 1H), 5.13-5.09 (m, 1H), 4.40-4.36 (m, 1H), 3.96-3.92 (m, 1H), 3.22-3.18 (m, 1H), 1.45 (d, J=7.0 Hz, 3H), 1.42-1.39 (m, 1H), 1.09 (d, J=6.5 Hz, 3H), 0.82-0.76 (m, 2H), 0.75-0.74 (m, 2H).

and peak 2, Example 306, (3R,4S)-3-cyclopropyl-1-(3-fluoro-6-(1-((2R,3 S)-2-methyloxetan-3-yl)-1H-pyrazol-4-yl)pyrazolo[1,5-a]pyrazin-4-yl)-4-methyl-2-oxopyrrolidine-3-carbonitrile and (3R,4S)-3-cyclopropyl-1-(3-fluoro-6-(1-((2S,3R)-2-methyloxetan-3-yl)-1H-pyrazol-4-yl)pyrazolo[1,5-a]pyrazin-4-yl)-4-methyl-2-oxopyrrolidine-3-carbonitrile (19.0 mg, 29.1% yield) as a white solid. The relative stereochemistry was determined by 2D nmr. LCMS m/z=436.1 [M+H]⁺; ¹H NMR (500 MHz, DMSO-d₆) δ=9.05-9.02 (m, 1H), 8.47 (s, 1H), 8.24-8.20 (m, 2H), 5.20-5.18 (m, 1H), 5.14-5.12 (m, 1H), 4.84-4.81 (m, 1H), 4.78-4.76 (m, 1H), 4.35-4.31 (m, 1H), 3.90-3.85 (m, 1H), 3.19-3.15 (m, 1H), 1.44 (d, J=6.0 Hz, 3H), 1.33 (d, J=7.0 Hz, 3H), 1.24-1.13 (m, 1H), 0.76-0.67 (m, 2H), 0.59-0.57 (m, 2H).

### Example 307 and 308

### (3R,4S)-1-(6-(1-((1s,3S)-3-cyano-3-methylcyclobutyl)-1H-pyrazol-4-yl)-3-fluoropyrazolo[1,5-a]pyrazin-4-yl)-3-cyclopropyl-4-methyl-2-oxopyrrolidine-3-carbonitrile and (3R,4S)-1-(6-(1-((1r,3R)-3-cyano-3-methylcyclobutyl)-1H-pyrazol-4-yl)-3-fluoropyrazolo[1,5-a]pyrazin-4-yl)-3-cyclopropyl-4-methyl-2-oxopyrrolidine-3-carbonitrile

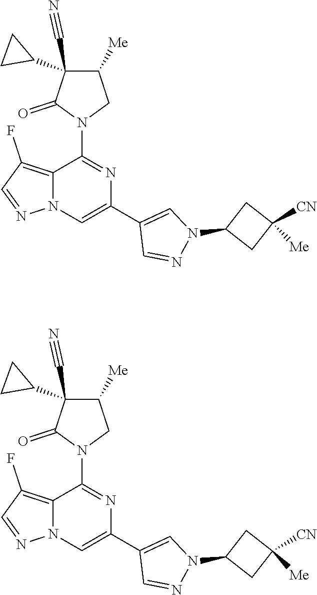 [SMILES: C[C@@H]1CN(c2nc(-c3cnn([C@H]4C[C@@](C)(C#N)C4)c3)cn3ncc(F)c23)C(=O)[C@]1(C#N)C1CC1 ; C[C@@H]1CN(c2nc(-c3cnn([C@H]4C[C@](C)(C#N)C4)c3)cn3ncc(F)c23)C(=O)[C@]1(C#N)C1CC1]

To a solution of 1-methyl-3-(4-(4,4,5,5-tetramethyl-1,3,2-dioxaborolan-2-yl)-1H-pyrazol-1-yl)cyclobutane-1-carbonitrile (Preparation 239, 110 mg, 0.383 mmol) in dioxane (4 mL) and water (0.8 mL) was added K₂CO₃ (158.8 mg, 1.15 mmol) and (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 127.8 mg, 0.383 mmol). Pd(dtbpf)Cl₂ (49.93 mg, 0.077 mmol) was added and the reaction was stirred at 90° C. under N₂ for 2 h. The mixture was concentrated under vacuum to give crude, which was purified by chromatography column on silica gel (PE/EtOAc=1/0 to 0/1) to give peak 1, as yellow oil and peak 2 as yellow oil.

Peak 1 was purified by Prep-HPLC-3 (44 to 74% gradient) to give Example 307, (3R,4S)-1-(6-(1-((1s,3S)-3-cyano-3-methylcyclobutyl)-1H-pyrazol-4-yl)-3-fluoropyrazolo[1,5-a]pyrazin-4-yl)-3-cyclopropyl-4-methyl-2-oxopyrrolidine-3-carbonitrile (8.7 mg, 5.0% yield) as a yellow solid. The relative stereochemistry was determined by 2D nmr. LCMS m/z=459.2 [M+H]⁺; ¹H NMR (400 MHz, CDCl₃) δ=8.26 (s, 1H), 7.92 (s, 1H), 7.87-7.81 (m, 2H), 5.10-4.92 (m, 1H), 4.36-4.24 (m, 1H), 3.81 (t, J=9.6 Hz, 1H), 3.23-3.13 (m, 1H), 3.12-3.03 (m, 2H), 2.86-2.77 (m, 2H), 1.68 (s, 3H), 1.45 (d, J=6.8 Hz, 3H), 1.26-1.15 (m, 1H), 0.92-0.76 (m, 1H).

Peak 2 was purified by Prep-HPLC-3 (42 to 72% gradient) to give, Example 308, (3R,4S)-1-(6-(1-((1r,3R)-3-cyano-3-methylcyclobutyl)-1H-pyrazol-4-yl)-3-fluoropyrazolo[1,5-a]pyrazin-4-yl)-3-cyclopropyl-4-methyl-2-oxopyrrolidine-3-carbonitrile (17.5 mg, 9.9% yield) as a yellow solid. The relative stereochemistry was determined by 2D nmr. LCMS m/z=459.2 [M+H]⁺; ¹H NMR (400 MHz, CDCl₃) δ=8.26 (s, 1H), 7.91 (d, J=3.6 Hz, 2H), 7.84 (d, J=4.0 Hz, 1H), 5.02-4.88 (m, 1H), 4.35-4.25 (m, 1H), 3.83 (t, J=10.0 Hz, 1H), 3.28-3.17 (m, 3H), 2.74-2.66 (m, 2H), 1.69 (s, 3H), 1.46 (d, J=7.2 Hz, 3H), 1.26-1.19 (m, 1H), 0.89-0.76 (m, 4H).

### Example 309

### (3R,4S)-1-(6-(1-((1r,3R)-3-cyano-3-fluorocyclobutyl)-1H-pyrazol-4-yl)-3-fluoropyrazolo[1,5-a]pyrazin-4-yl)-3-cyclopropyl-4-methyl-2-oxopyrrolidine-3-carbonitrile

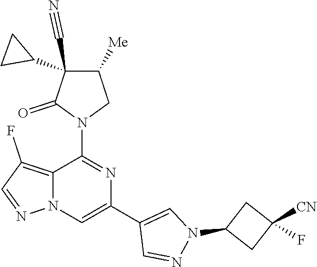 [SMILES: C[C@@H]1CN(c2nc(-c3cnn([C@H]4C[C@@](F)(C#N)C4)c3)cn3ncc(F)c23)C(=O)[C@]1(C#N)C1CC1]

To a solution of 1-fluoro-3-(4-(4,4,5,5-tetramethyl-1,3,2-dioxaborolan-2-yl)-1H-pyrazol-1-yl)cyclobutane-1-carbonitrile (Preparation 240, 300 mg, 1.03 mmol) in dioxane (10 mL) and water (2 mL) was added K₂CO₃ (427.3 mg, 3.09 mmol) and (3R,4S)-1-(6-chloro-3-fluoropyrazolo[1,5-a]pyrazin-4-yl)-3-cyclopropyl-4-methyl-2-oxopyrrolidine-3-carbonitrile (Preparation 125, 343.9 mg, 1.03 mmol) at 20° C. Pd(dtbpf)Cl₂ (134.3 mg, 0.206 mmol) was added and the reaction was stirred at 90° C. under N₂ for 2 h. The mixture was poured into water (50 mL) and extracted with DCM (50 mL×3). The combined organic layers were washed with brine (100 mL), dried over Na₂SO₄, filtered and concentrated under vacuum to give the crude, which was purified by Prep-HPLC-3 (50 to 80% gradient) to give a yellow solid (150 mg, 31.1% yield). This compound was purified by SFC (column: DAICEL CHIRALCEL OD-H (250 mm×30 mm, 5 μm), 30% isocratic (0.1% NH₃·H₂O, EtOH as mobile phase), Flow Rate (mL/min): 80) to give (3R,4S)-1-(6-(1-((1r,3R)-3-cyano-3-fluorocyclobutyl)-1H-pyrazol-4-yl)-3-fluoropyrazolo[1,5-a]pyrazin-4-yl)-3-cyclopropyl-4-methyl-2-oxopyrrolidine-3-carbonitrile (45.6 mg, 30.4% yield) as a yellow solid. The relative stereochemistry was confirmed by 2D nmr. LCMS m/z=463.2 [M+H]⁺; ¹H NMR (400 MHz, CDCl₃) δ: =8.26 (d, J=1.2 Hz, 1H), 7.96 (s, 1H), 7.88 (s, 1H), 7.84 (d, J=4.0 Hz, 1H), 5.20-5.10 (m, 1H), 4.33-4.24 (m, 1H), 3.81 (t, J=9.6 Hz, 1H), 3.54-3.40 (m, 2H), 3.24-3.13 (m, 3H), 1.45 (d, J=6.8 Hz, 3H), 1.24-1.17 (m, 1H), 0.90-0.76 (m, 4H).

### Example 310

### (3R,4S)-1-(3-fluoro-6-(1-methyl-1H-pyrazol-4-yl)pyrazolo[1,5-a]pyrazin-4-yl)-3-isopropyl-4-methyl-2-oxopyrrolidine-3-carbonitrile

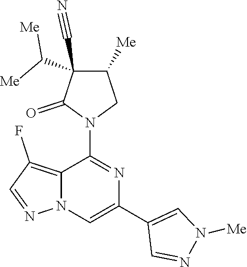 [SMILES: CC(C)[C@@]1(C#N)C(=O)N(c2nc(-c3cnn(C)c3)cn3ncc(F)c23)C[C@H]1C]

N₂ was bubbled through a mixture of (3R,4S)-1-(6-chloro-3-fluoropyrazolo[1,5-a]pyrazin-4-yl)-3-isopropyl-4-methyl-2-oxopyrrolidine-3-carbonitrile (Preparation 266, 180 mg, 0.536 mmol), 1-methyl-4-(4,4,5,5-tetramethyl-1,3,2-dioxaborolan-2-yl)-1H-pyrazole (150 mg, 0.721 mmol), K₂CO₃ (222.3 mg, 1.61 mmol) and Pd(dtbpf)Cl₂ (34.94 mg, 0.054 mmol) in dioxane (10 mL) and water (2 mL) and the reaction mixture was stirred at 90° C. for 2 h. The reaction mixture was concentrated in vacuum to give the crude, which was purified by prep HPLC-3 (32 to 62% gradient) to give (3R,4S)-1-(3-fluoro-6-(1-methyl-1H-pyrazol-4-yl)pyrazolo[1,5-a]pyrazin-4-yl)-3-isopropyl-4-methyl-2-oxopyrrolidine-3-carbonitrile (58.7 mg, 28.7% yield) as a white solid. LCMS m/z=382.1 [M+H]⁺; ¹H NMR (500 MHz, MeOD-d₄) δ=8.65 (s, 1H), 8.15 (s, 1H), 8.07-7.92 (m, 2H), 4.40 (dd, J=7.0 Hz, J₂=10.5 Hz, 1H), 3.96 (s, 3H), 3.90 (dd, J₁=6.0 Hz, J₂ 10.0 Hz, 1H), 3.30-3.10 (m, 1H), 2.40-2.34 (m, 1H), 1.38 (d, J=7.0 Hz, 3H), 1.33 (d, J=7.0 Hz, 3H), 1.29 (d, J=6.5 Hz, 3H).

### Example 315

### (3R,4S)-3-cyclopropyl-4-methyl-1-(6-(1-(2-methyl-2-azaspiro[3.3]heptan-6-yl)-1H-pyrazol-4-yl)pyrrolo[1,2-b]pyridazin-4-yl)-2-oxopyrrolidine-3-carbonitrile

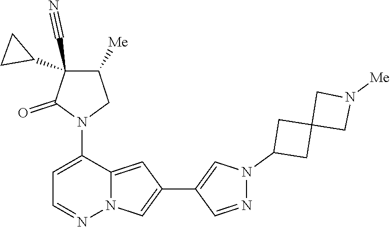 [SMILES: C[C@@H]1CN(c2ccnn3cc(-c4cnn(C5CC6(C5)CN(C)C6)c4)cc23)C(=O)[C@]1(C#N)C1CC1]

Part A: To a solution of tert-butyl 6-(4-(4-((3R,4S)-3-cyano-3-cyclopropyl-4-methyl-2-oxopyrrolidin-1-yl)pyrrolo[1,2-b]pyridazin-6-yl)-1H-pyrazol-1-yl)-2-azaspiro[3.3]heptane-2-carboxylate (Preparation 276, 50 mg, 0.092 mmol) in DCM (2 mL) and 1,1,1,3,3,3-hexafluoropropan-2-ol (2 mL) was added TFA (1 mL) and the solution was stirred at 20° C. for 30 min. DIPEA (1 mL) was added and the reaction mixture was concentrated under vacuum to give (3R,4S)-1-(6-(1-(2-azaspiro[3.3]heptan-6-yl)-1H-pyrazol-4-yl)pyrrolo[1,2-b]pyridazin-4-yl)-3-cyclopropyl-4-methyl-2-oxopyrrolidine-3-carbonitrile (50 mg, crude) as yellow oil. LCMS m/z=442.1 [M+H]⁺

Part B: To a solution of (3R,4S)-1-(6-(1-(2-azaspiro[3.3]heptan-6-yl)-1H-pyrazol-4-yl)pyrrolo[1,2-b]pyridazin-4-yl)-3-cyclopropyl-4-methyl-2-oxopyrrolidine-3-carbonitrile (50 mg, 0.113 mmol) in MeOH (2 mL) was added CH₂O (135.84 mg, 0.113 mmol) and the solution was stirred at 20° C. for 30 mins. NaBH₃CN (21.35 mg, 0.340 mmol) was added and the reaction was stirred at 20° C. for 1 h. The reaction mixture was concentrated in vacuo and the crude product was purified by prep HPLC-3 (45 to 75% gradient) to give (3R,4S)-3-cyclopropyl-4-methyl-1-(6-(1-(2-methyl-2-azaspiro[3.3]heptan-6-yl)-1H-pyrazol-4-yl)pyrrolo[1,2-b]pyridazin-4-yl)-2-oxopyrrolidine-3-carbonitrile (27.1 mg, 49.0% yield) as a white solid. LCMS m/z=456.4 [M+H]⁺; ¹H NMR (500 MHz, MeOD-d₄) δ=8.08-8.01 (m, 3H), 7.86 (s, 1H), 6.90-6.80 (m, 2H), 4.82-4.73 (m, 1H), 4.17-4.10 (m, 1H), 4.02 (t, J=10.0 Hz, 1H), 3.48-3.42 (m, 2H), 3.35 (s, 2H), 3.21-3.11 (m, 1H), 2.76-2.68 (m, 4H), 2.38-2.31 (m, 3H), 1.43 (d, J=7.0 Hz, 3H), 1.19 (d, J=6.5 Hz, 1H), 0.84-0.80 (m, 1H), 0.78-0.70 (m, 3H).

### Example 316

### (3R,4S)-3-cyclopropyl-4-methyl-1-(6-(1-(1-methylazetidin-3-yl)-1H-pyrazol-4-yl)pyrrolo[1,2-b]pyridazin-4-yl)-2-oxopyrrolidine-3-carbonitrile

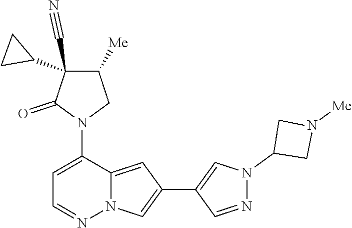 [SMILES: C[C@@H]1CN(c2ccnn3cc(-c4cnn(C5CN(C)C5)c4)cc23)C(=O)[C@]1(C#N)C1CC1]

(3R,4S)-3-Cyclopropyl-4-methyl-1-(6-(1-(1-methylazetidin-3-yl)-1H-pyrazol-4-yl)pyrrolo[1,2-b]pyridazin-4-yl)-2-oxopyrrolidine-3-carbonitrile was obtained, as a yellow solid, 28.1 mg, 17% yield, from tert-butyl 3-(4-(4-((3R,4S)-3-cyano-3-cyclopropyl-4-methyl-2-oxopyrrolidin-1-yl)pyrrolo[1,2-b]pyridazin-6-yl)-1H-pyrazol-1-yl)azetidine-1-carboxylate (Preparation 278), following a similar 2 step procedure to that described in Example 315. LCMS m/z=416.1 [M+H]⁺; 1H NMR (500 MHz, MeOD-d₄) δ=8.16 (s, 1H), 8.11-8.03 (m, 2H), 7.92 (s, 1H), 6.94-6.82 (m, 2H), 5.06 (d, J=7.0 Hz, 1H), 4.16-4.06 (m, 1H), 4.03 (t, J=10.0 Hz, 1H), 3.95-3.85 (m, 2H), 3.68-3.62 (m, 2H), 3.21-3.14 (m, 1H), 2.50 (s, 3H), 1.49-1.40 (m, 4H), 0.85-0.70 (m, 4H).

### Example 317

### (3R,4S)-3-cyclopropyl-4-methyl-1-(6-(4-(4-methylmorpholin-2-yl)-1H-pyrazol-1-yl)pyrrolo[1,2-b]pyridazin-4-yl)-2-oxopyrrolidine-3-carbonitrile

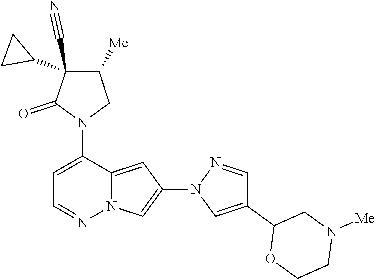 [SMILES: C[C@@H]1CN(c2ccnn3cc(-n4cc(C5CN(C)CCO5)cn4)cc23)C(=O)[C@]1(C#N)C1CC1]

Part A: tert-Butyl 2-(1-(4-((3R,4S)-3-cyano-3-cyclopropyl-4-methyl-2-oxopyrrolidin-1-yl)pyrrolo[1,2-b]pyridazin-6-yl)-1H-pyrazol-4-yl)morpholine-4-carboxylate (Preparation 277, 32 mg, 0.06 mmol) was added to HCl/EtOAc (6 mL) and the reaction mixture was stirred at 25° C. for 2 h. The reaction mixture was concentrated in vacuo to give (3R,4S)-3-cyclopropyl-4-methyl-1-(6-(4-(morpholin-2-yl)-1H-pyrazol-1-yl)pyrrolo[1,2-b]pyridazin-4-yl)-2-oxopyrrolidine-3-carbonitrile hydrochloride (26 mg, crude, HCl) as a light brown solid which was used without further purification. LCMS m/z=432.2 [M+H]⁺

Part B: To a solution of (3R,4S)-3-cyclopropyl-4-methyl-1-(6-(4-(morpholin-2-yl)-1H-pyrazol-1-yl)pyrrolo[1,2-b]pyridazin-4-yl)-2-oxopyrrolidine-3-carbonitrile hydrochloride (26 mg, 0.060 mmol) and paraformaldehyde (35.0 mg, 1.16 mmol) in MeOH (5 mL) was added TEA (37.96 mg, 0.375 mmol) and NaCNBH₃ (11.36 mg, 0.181 mmol) and the reaction mixture was stirred at 20° C. for 2 h. The reaction mixture was concentrated in vacuo and the crude product was purified by prep-HPLC-16 (25 to 41% gradient) to give (3R,4S)-3-cyclopropyl-4-methyl-1-(6-(4-(4-methylmorpholin-2-yl)-1H-pyrazol-1-yl)pyrrolo[1,2-b]pyridazin-4-yl)-2-oxopyrrolidine-3-carbonitrile (7 mg, 26.1% yield) as a light gray solid. LCMS m/z=446.2 [M+H]⁺; ¹H NMR (400 MHz, MeOD-d₄) δ=8.31 (s, 1H), 8.21 (d, J=2.0 Hz, 1H), 8.17 (d, J=4.8 Hz, 1H), 7.76 (s, 1H), 7.01-6.97 (m, 2H), 4.80-4.76 (m, 1H), 4.15-4.11 (m, 2H), 4.04-4.01 (m, 1H), 4.00-3.90 (m, 1H), 3.39-3.36 (m, 1H), 3.22-3.17 (m, 2H), 2.84-2.79 (m, 2H), 2.72 (s, 3H), 1.50-1.42 (m, 4H), 0.88-0.71 (m, 4H).

### Example 318

### (3R,4S)-3-cyclopropyl-4-methyl-1-(6-(5-methyl-1H-pyrazol-3-yl)pyrazolo[1,5-a]pyridin-4-yl)-2-oxopyrrolidine-3-carbonitrile

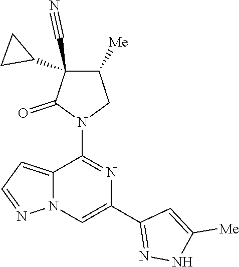 [SMILES: Cc1cc(-c2cn3nccc3c(N3C[C@@H](C)[C@@](C#N)(C4CC4)C3=O)n2)n[nH]1]

To a solution of (3R,4S)-1-(6-bromopyrazolo[1,5-a]pyridin-4-yl)-3-cyclopropyl-4-methyl-2-oxopyrrolidine-3-carbonitrile (Preparation 264, 35 mg, 0.097 mmol) in dioxane (1.5 mL) and water (0.3 mL) was added K₂CO₃ (40.40 mg, 0.292 mmol) and 5-methyl-3-(4,4,5,5-tetramethyl-1,3,2-dioxaborolan-2-yl)-1H-pyrazole (30.41 mg, 0.146 mmol) at 20° C. Pd(dtbpf)Cl₂ (12.70 mg, 0.0195 mmol) was added and the reaction was stirred at 90° C. under N₂ for 2 h. The mixture was poured into water (50 mL) and extracted with DCM (50 mL×3). The combined organic layers were washed with brine (100 mL), dried over Na₂SO₄, filtered and concentrated under vacuum. The crude product was purified by Prep-HPLC-18 (gradient: 38 to 58%) to give (3R,4S)-3-cyclopropyl-4-methyl-1-(6-(5-methyl-1H-pyrazol-3-yl)pyrazolo[1,5-a]pyridin-4-yl)-2-oxopyrrolidine-3-carbonitrile (19.4 mg, 55.3% yield) as a yellow solid. LCMS m/z=361.2 [M+H]⁺; ¹H NMR: (500 MHz, CDCl₃) δ: =8.84 (s, 1H), 7.99 (d, J=2.5 Hz, 1H), 7.64 (s, 1H), 6.49 (d, J=2.5 Hz, 1H), 6.38 (s, 1H), 4.03-3.94 (m, 1H), 3.77-3.67 (m, 1H), 3.16-3.13 (m, 1H), 2.40 (s, 3H), 1.43 (d, J=6.5 Hz, 3H), 1.23-1.16 (m, 1H), 0.88-0.82 (m, 2H), 0.81-0.76 (m, 2H).

### Example 319

### (3R,4S)-1-(6-(1-(2-oxaspiro[3.3]heptan-6-yl)-1H-pyrazol-4-yl)pyrrolo[1,2-b]pyridazin-4-yl)-3-cyclopropyl-4-methyl-2-oxopyrrolidine-3-carbonitrile

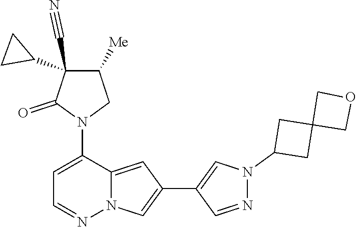 [SMILES: C[C@@H]1CN(c2ccnn3cc(-c4cnn(C5CC6(COC6)C5)c4)cc23)C(=O)[C@]1(C#N)C1CC1]

N₂ was bubbled through a mixture of (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 100 mg, 0.246 mmol), 4-bromo-1-(2-oxaspiro[3.3]heptan-6-yl)-1H-pyrazole (Preparation 214, 100 mg, 0.411 mmol), K₂CO₃ (102.1 mg, 0.738 mmol) and Pd(dtbpf)Cl₂ (16.04 mg, 0.025 mmol) in dioxane (10 mL) and water (2 mL) and the reaction mixture was stirred at 90° C. for 2 h. The reaction mixture was concentrated in vacuum to give the crude, which was purified by prep HPLC-3 (31 to 61% gradient) to give (3R,4S)-1-(6-(1-(2-oxaspiro[3.3]heptan-6-yl)-1H-pyrazol-4-yl)pyrrolo[1,2-b]pyridazin-4-yl)-3-cyclopropyl-4-methyl-2-oxopyrrolidine-3-carbonitrile (67.8 mg, 62.3% yield) as a yellow solid. LCMS m/z=443.2 [M+H]⁺; 1H NMR (500 MHz, MeOD-d₄) δ=8.14-7.80 (m, 4H), 6.91-6.77 (m, 2H), 4.84 (s, 2H), 4.76-4.68 (m, 3H), 4.16-4.11 (m, 1H), 4.00 (t, J=10.0 Hz, 1H), 3.16 (m, 1H), 2.91-2.84 (m, 2H), 2.80-2.74 (m, 2H), 1.46-1.39 (m, 4H), 0.85-0.69 (m, 4H).

### Example 320

### (3R,4S)-1-(6-(1-(3-cyanobicyclo[1.1.1]pentan-1-yl)-1H-pyrazol-4-yl)pyrrolo[1,2-b]pyridazin-4-yl)-3-cyclopropyl-4-methyl-2-oxopyrrolidine-3-carbonitrile

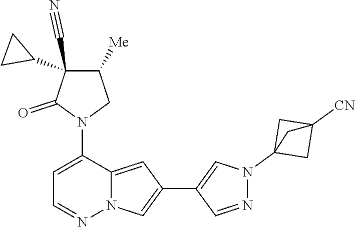 [SMILES: C[C@@H]1CN(c2ccnn3cc(-c4cnn(C56CC(C#N)(C5)C6)c4)cc23)C(=O)[C@]1(C#N)C1CC1]

To a solution of (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 51.19 mg, 0.126 mmol) and 3-(4-bromo-1H-pyrazol-1-yl)bicyclo[1.1.1]pentane-1-carbonitrile (Preparation 225, 30 mg, 126.01 mmol) in a mixture of dioxane (2 mL) and water (0.5 mL) was added K₂CO₃ (34.83 mg, 0.252 mmol) and Pd(dtbpf)Cl₂ (8.21 mg, 0.0126 mmol) at 15° C. and the reaction mixture was stirred at 90° C. under N₂ for 3 h. The reaction mixture was concentrated in vacuum and the crude product was purified by Prep-HPLC-16 (51 to 61% gradient) to give (3R,4S)-1-(6-(1-(3-cyanobicyclo[1.1.1]pentan-1-yl)-1H-pyrazol-4-yl)pyrrolo[1,2-b]pyridazin-4-yl)-3-cyclopropyl-4-methyl-2-oxopyrrolidine-3-carbonitrile (18 mg, 32.7% yield) as a white solid. LCMS m/z=438.2 [M+H]⁺; ¹H NMR (500 MHz, MeOD-d₄) δ=8.08 (s, 1H), 8.05-8.02 (m, 2H), 7.91 (s, 1H), 6.87-6.85 (m, 1H), 6.83 (s, 1H), 4.14-4.09 (m, 1H), 4.10-3.97 (m, 1H), 3.17-3.11 (m, 1H), 2.81 (s, 6H), 1.42-1.40 (m, 4H), 0.81-0.69 (m, 4H).

### Example 321

### (3R,4S)-3-cyclopropyl-1-(6-(3-fluoro-1-(oxetan-3-yl)-1H-pyrazol-4-yl)pyrrolo[1,2-b]pyridazin-4-yl)-4-methyl-2-oxopyrrolidine-3-carbonitrile

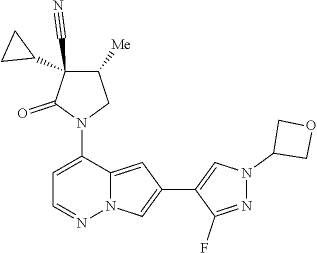 [SMILES: C[C@@H]1CN(c2ccnn3cc(-c4cn(C5COC5)nc4F)cc23)C(=O)[C@]1(C#N)C1CC1]

(3R,4S)-3-Cyclopropyl-1-(6-(3-fluoro-1-(oxetan-3-yl)-1H-pyrazol-4-yl)pyrrolo[1,2-b]pyridazin-4-yl)-4-methyl-2-oxopyrrolidine-3-carbonitrile was obtained as a yellow solid, 43.1 mg, 44.1% yield, from 4-bromo-3-fluoro-1-(oxetan-3-yl)-1H-pyrazole (Preparation 218) and (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69) following a similar procedure to that described in Example 320, except the crude product was purified by Prep HPLC-21 (gradient: 42 to 72%). LCMS m/z=421.1 [M+H]⁺; ¹H NMR (400 MHz, MeOD-d₄) δ=8.10-8.00 (m, 3H), 6.89 (d, J=5.2 Hz, 1H), 6.82 (d, J=1.6 Hz, 1H), 5.51-5.39 (m, 1H), 5.04 (d, J=7.2 Hz, 4H), 4.17-4.12 (m, 1H), 3.98 (t, J=9.6 Hz, 1H), 3.24-3.12 (m, 1H), 1.48-1.37 (m, 4H), 0.85-0.75 (m, 2H), 0.74-0.70 (m, 2H).

### Example 322

### (3R,4S)-3-cyclopropyl-1-(6-(1-(1-(hydroxymethyl)cyclopropyl)-1H-pyrazol-4-yl)pyrrolo[1,2-b]pyridazin-4-yl)-4-methyl-2-oxopyrrolidine-3-carbonitrile

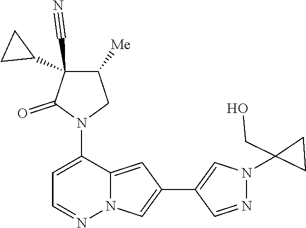 [SMILES: C[C@@H]1CN(c2ccnn3cc(-c4cnn(C5(CO)CC5)c4)cc23)C(=O)[C@]1(C#N)C1CC1]

To a solution of (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 70 mg, 0.172 mmol) in dioxane (3 mL) and water (0.6 mL) was added K₂CO₃ (71.44 mg, 0.517 mmol) and (1-(4-bromo-1H-pyrazol-1-yl)cyclopropyl)methanol (37.40 mg, 0.172 mmol) at 20° C. Pd(dtbpf)Cl₂ (22.46 mg, 0.0345 mmol) was added and the reaction was stirred at 90° C. under N₂ for 2 h. The reaction was quenched with water (20 mL) and extracted with DCM (15 mL×3). The combined organic extracts were dried over Na₂SO₄, filtered and evaporated under vacuum. The residue was purified by Prep-HPLC-19 (gradient: 44 to 64%) to give (3R,4S)-3-cyclopropyl-1-(6-(1-(1-(hydroxymethyl)cyclopropyl)-1H-pyrazol-4-yl)pyrrolo[1,2-b]pyridazin-4-yl)-4-methyl-2-oxopyrrolidine-3-carbonitrile (18.7 mg, 26.1% yield) as a yellow solid. LCMS m/z=417.3 [M+H]⁺; ¹H NMR (400 MHz, MeOD-d₄) δ=8.10 (s, 1H), 8.06-8.04 (m, 2H), 7.88 (s, 1H), 6.89-6.85 (m, 2H), 4.16-4.13 (m, 1H), 4.05-3.95 (m, 1H), 3.80 (s, 1H), 3.19-3.15 (m, 1H), 1.46-1.43 (m, 5H), 1.29-1.28 (m, 2H), 1.17-1.16 (m, 2H), 0.82-0.70 (m, 5H).

### Example 323

### (3R,4S)-3-cyclopropyl-1-(6-(1-(1-ethylcyclopropyl)-1H-pyrazol-4-yl)pyrrolo[1,2-b]pyridazin-4-yl)-4-methyl-2-oxopyrrolidine-3-carbonitrile

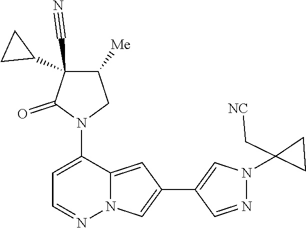 [SMILES: C[C@@H]1CN(c2ccnn3cc(-c4cnn(C5(CC#N)CC5)c4)cc23)C(=O)[C@]1(C#N)C1CC1]

(3R,4S)-3-Cyclopropyl-1-(6-(1-(1-ethylcyclopropyl)-1H-pyrazol-4-yl)pyrrolo[1,2-b]pyridazin-4-yl)-4-methyl-2-oxopyrrolidine-3-carbonitrile was obtained as a yellow solid, 34.8 mg, 29% yield, from 2-(1-(4-bromo-1H-pyrazol-1-yl)cyclopropyl)acetonitrile (Preparation 269) and (4-((3R,4S)-3-cyano-3-cyclopropyl-4-methyl-2-oxopyrrolidin-1-yl)pyrrolo[1,2-b]pyridazin-6-yl)boronic acid (Preparation 267) following a similar procedure to that described in Example 322, except the crude product was purified by Prep-HPLC-3 (32 to 62% gradient). LCMS m/z=426.3 [M+H]⁺; ¹H NMR (400 MHz, CDCl₃) δ: =8.00 (d, J=5.2 Hz, 1H), 7.91 (d, J=1.6 Hz, 1H), 7.81 (d, J=6.4 Hz, 2H), 6.88 (d, J=5.2 Hz, 1H), 6.60 (d, J=1.6 Hz, 1H), 4.18-4.08 (m, 1H), 3.89 (t, J=9.6 Hz, 1H), 3.15-3.07 (m, 1H), 2.99 (d, J=2.8 Hz, 2H), 1.52-1.49 (m, 2H), 1.46 (d, J=6.8 Hz, 3H), 1.29-1.26 (m, 2H), 1.23-1.17 (m, 1H), 0.84-0.76 (m, 4H).

### Example 324

### (3R,4S)-1-(6-(1-((1-cyanocyclopropyl)methyl)-1H-pyrazol-4-yl)pyrrolo[1,2-b]pyridazin-4-yl)-3-cyclopropyl-4-methyl-2-oxopyrrolidine-3-carbonitrile

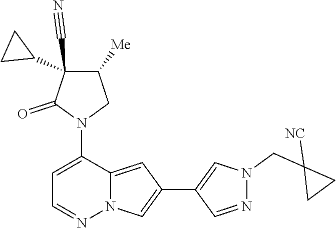 [SMILES: C[C@@H]1CN(c2ccnn3cc(-c4cnn(CC5(C#N)CC5)c4)cc23)C(=O)[C@]1(C#N)C1CC1]

(3R,4S)-1-(6-(1-((1-Cyanocyclopropyl)methyl)-1H-pyrazol-4-yl)pyrrolo[1,2-b]pyridazin-4-yl)-3-cyclopropyl-4-methyl-2-oxopyrrolidine-3-carbonitrile was obtained as a yellow solid, 37.4 mg, 49.7% yield, from (4-((3R,4S)-3-cyano-3-cyclopropyl-4-methyl-2-oxopyrrolidin-1-yl)pyrrolo[1,2-b]pyridazin-6-yl)boronic acid (Preparation 267) and 1-((4-bromo-1H-pyrazol-1-yl)methyl)cyclopropane-1-carbonitrile, following the procedure described in Example 323. LCMS m/z=426.2 [M+H]⁺; ¹H NMR (400 MHz, CDCl₃) δ: =8.00 (d, J=4.8 Hz, 1H), 7.93 (d, J=1.6 Hz, 1H), 7.81 (d, J=13.2 Hz, 2H), 6.90 (d, J=5.2 Hz, 1H), 6.62 (d, J=1.6 Hz, 1H), 4.31-4.22 (m, 2H), 4.17-4.10 (m, 1H), 3.90 (t, J=9.6 Hz, 1H), 3.16-3.05 (m, 1H), 1.46 (d, J=6.8 Hz, 3H), 1.44-1.40 (m, 2H), 1.29-1.24 (m, 2H), 1.23-1.17 (m, 1H), 0.85-0.75 (m, 4H).

### Example 325

### (3R,4S)-3-cyclopropyl-4-methyl-1-(6-(1-((3-methyloxetan-3-yl)methyl)-1H-pyrazol-4-yl)pyrrolo[1,2-b]pyridazin-4-yl)-2-oxopyrrolidine-3-carbonitrile

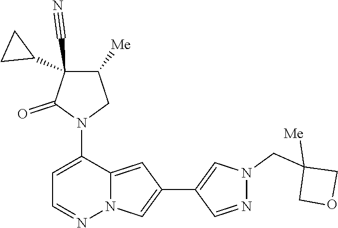 [SMILES: C[C@@H]1CN(c2ccnn3cc(-c4cnn(CC5(C)COC5)c4)cc23)C(=O)[C@]1(C#N)C1CC1]

To a solution of (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 60 mg, 0.148 mmol) in dioxane (1 mL) and H₂O (0.2 mL) was added K₂CO₃ (61.23 mg, 0.443 mmol) and 4-bromo-1-((3-methyloxetan-3-yl)methyl)-1H-pyrazole (37.54 mg, 0.162 mmol) at 20° C. Pd(dtbpf)Cl₂ (9.62 mg, 0.0148 mmol) was added and the reaction was stirred at 90° C. under N₂ for 2 h. The mixture was concentrated under vacuum and the crude product was purified by chromatography column on silica gel (PE/EtOAc=0/1). The product was further purified by Prep-HPLC-20 (gradient: 40 to 60%) to give (3R,4S)-3-cyclopropyl-4-methyl-1-(6-(1-((3-methyloxetan-3-yl)methyl)-1H-pyrazol-4-yl)pyrrolo[1,2-b]pyridazin-4-yl)-2-oxopyrrolidine-3-carbonitrile (28 mg, 44.0% yield) as a green solid. LCMS m/z=431.2 [M+H]⁺; ¹H NMR (400 MHz, CDCl₃) δ: =8.01 (d, J=5.2 Hz, 1H), 7.92 (s, 1H), 7.77 (s, 1H), 7.63 (s, 1H), 6.86-6.79 (m, 1H), 6.61 (s, 1H), 4.77 (d, J=6.0 Hz, 2H), 4.45 (d, J=6.0 Hz, 2H), 4.39 (s, 2H), 4.16-4.10 (m, 1H), 3.90-3.85 (m, 1H), 3.15-3.08 (m, 1H), 1.46 (d, J=7.6 Hz, 3H), 1.32 (s, 3H), 1.22-1.19 (m, 1H), 0.85-0.78 (m, 4H)

### Example 326

### (3R,4S)-1-(6-(1-((1r,3R)-3-cyano-3-fluorocyclobutyl)-1H-pyrazol-4-yl)pyrrolo[1,2-b]pyridazin-4-yl)-3-cyclopropyl-4-methyl-2-oxopyrrolidine-3-carbonitrile

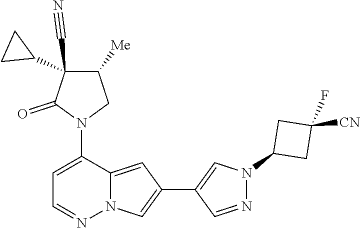 [SMILES: C[C@@H]1CN(c2ccnn3cc(-c4cnn([C@H]5C[C@@](F)(C#N)C5)c4)cc23)C(=O)[C@]1(C#N)C1CC1]

To a solution of (1r,3r)-3-(4-bromo-1H-pyrazol-1-yl)-1-fluorocyclobutane-1-carbonitrile (Preparation 221, 150 mg, 0.615 mmol) in water (1 mL) and dioxane (6 mL) was added K₂CO₃ (254.8 mg, 1.84 mmol) and (4-((3R,4S)-3-cyano-3-cyclopropyl-4-methyl-2-oxopyrrolidin-1-yl)pyrrolo[1,2-b]pyridazin-6-yl)boronic acid (Preparation 267, 199.2 mg, 0.615 mmol) at 20° C. Pd(dtbpf)Cl₂ (80.11 mg, 0.123 mmol) was added and the reaction was stirred at 90° C. under N₂ for 2 h. The mixture was poured into water (20 mL) and extracted with DCM (20 mL×3). The combined organic layers were washed with brine (100 mL), dried over Na₂SO₄, filtered and concentrated under vacuum. The crude product was purified by Prep-HPLC-3 (38 to 68% gradient) to give a yellow solid (150 mg, 48.3% yield). LCMS m/z=444.2 [M+H]⁺

This compound (150 mg, 0.338 mmol) was further purified by SFC (column: DAICEL CHIRALCEL OJ (250 mm×30 mm, 10 μm), 40% EtOH (0.1% NH₃·H₂O) EtOH as mobile phase, Flow Rate (mL/min): 80) to give (3R,4S)-1-(6-(1-((1r,3R)-3-cyano-3-fluorocyclobutyl)-1H-pyrazol-4-yl)pyrrolo[1,2-b]pyridazin-4-yl)-3-cyclopropyl-4-methyl-2-oxopyrrolidine-3-carbonitrile (74.2 mg, 49.5% yield) as a yellow solid. The relative stereochemistry was confirmed by 2D nmr. LCMS m/z=444.2 [M+H]⁺; ¹H NMR (400 MHz, CDCl₃) δ=8.00 (d, J=4.8 Hz, 1H), 7.90 (d, J=1.6 Hz, 1H), 7.84 (s, 1H), 7.66 (s, 1H), 6.82 (d, J=5.2 Hz, 1H), 6.60 (d, J=1.6 Hz, 1H), 5.18-5.07 (m, 1H), 4.17-4.06 (m, 1H), 3.86 (t, J=9.6 Hz, 1H), 3.54-3.40 (m, 2H), 3.22-3.07 (m, 3H), 1.45 (d, J=6.8 Hz, 3H), 1.23-1.16 (m, 1H), 0.85-0.77 (m, 4H).

### Example 327 and 328

### (3R,4S)-3-cyclopropyl-1-(6-(1-((R)-5,5-difluorotetrahydro-2H-pyran-3-yl)-1H-pyrazol-4-yl)pyrrolo[1,2-b]pyridazin-4-yl)-4-methyl-2-oxopyrrolidine-3-carbonitrile and (3R,4S)-3-cyclopropyl-1-(6-(1-((S)-5,5-difluorotetrahydro-2H-pyran-3-yl)-1H-pyrazol-4-yl)pyrrolo[1,2-b]pyridazin-4-yl)-4-methyl-2-oxopyrrolidine-3-carbonitrile

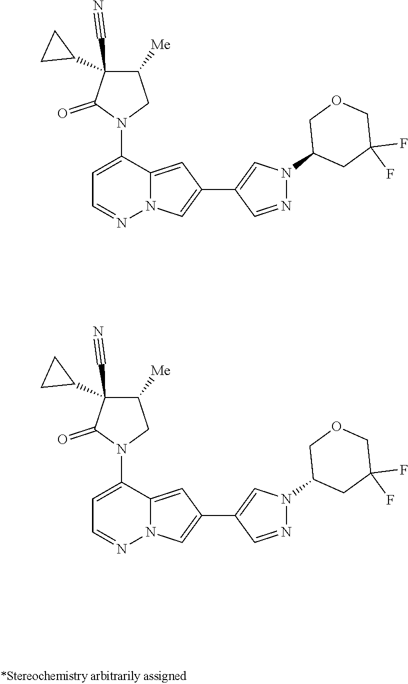 [SMILES: C[C@@H]1CN(c2ccnn3cc(-c4cnn([C@H]5COCC(F)(F)C5)c4)cc23)C(=O)[C@]1(C#N)C1CC1 ; C[C@@H]1CN(c2ccnn3cc(-c4cnn([C@@H]5COCC(F)(F)C5)c4)cc23)C(=O)[C@]1(C#N)C1CC1]

Part A: To a solution of 4-bromo-1-(5,5-difluorotetrahydro-2H-pyran-3-yl)-1H-pyrazole (Preparation 228, 130 mg, 0.487 mmol) in dioxane (5 mL) and water (1 mL) was added K₂CO₃ (201.8 mg, 1.46 mmol) and (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 217.5 mg, 0.535 mmol) at 20° C. Pd(dtbpf)Cl₂ (63.45 mg, 0.097 mmol) was added and the reaction was stirred at 90° C. under N₂ for 2 h. The mixture was concentrated under vacuum and the crude product was purified by column chromatography on silica gel (PE/EtOAc=1/0 to 1/1) to give (3R,4S)-3-cyclopropyl-1-(6-(1-(5,5-difluorotetrahydro-2H-pyran-3-yl)-1H-pyrazol-4-yl)pyrrolo[1,2-b]pyridazin-4-yl)-4-methyl-2-oxopyrrolidine-3-carbonitrile (100 mg, 41.4% yield) as yellow oil. LCMS m/z=467.1 [M+H]⁺

Part B: (3R,4S)-3-Cyclopropyl-1-(6-(1-(5,5-difluorotetrahydro-2H-pyran-3-yl)-1H-pyrazol-4-yl)pyrrolo[1,2-b]pyridazin-4-yl)-4-methyl-2-oxopyrrolidine-3-carbonitrile (100 mg, 0.214 mmol) was purified by SFC (column: DAICEL CHIRALPAK AS (250 mm×30 mm, 10 m), 25% (0.1% NH₃·H₂O, EtOH) as mobile phase, Flow Rate (mL/min): 70) to give, Example 327, (3R,4S)-3-cyclopropyl-1-(6-(1-((R)-5,5-difluorotetrahydro-2H-pyran-3-yl)-1H-pyrazol-4-yl)pyrrolo[1,2-b]pyridazin-4-yl)-4-methyl-2-oxopyrrolidine-3-carbonitrile (stereochemistry arbitrarily assigned) (35.7 mg, 35.7% yield) as a yellow solid. LCMS m/z=467.1 [M+H]⁺; ¹H NMR (400 MHz, MeOD-d₄) δ=8.00 (d, J=5.2 Hz, 1H), 7.91 (s, 1H), 7.81 (s, 1H), 7.77 (s, 1H), 6.84 (d, J=5.2 Hz, 1H), 6.61 (s, 1H), 4.78-4.67 (m, 1H), 4.19-4.09 (m, 2H), 4.02-3.94 (m, 1H), 3.90-3.84 (m, 2H), 3.74-3.63 (m, 1H), 3.15-3.06 (m, 1H), 2.77-2.67 (m, 2H), 1.45 (d, J=6.8 Hz, 3H), 1.22-1.16 (m, 1H), 0.84-0.76 (m, 4H).

Further elution provided Example 328, (3R,4S)-3-cyclopropyl-1-(6-(1-((S)-5,5-difluorotetrahydro-2H-pyran-3-yl)-1H-pyrazol-4-yl)pyrrolo[1,2-b]pyridazin-4-yl)-4-methyl-2-oxopyrrolidine-3-carbonitrile (stereochemistry arbitrarily assigned) (27.8 mg, 27.8% yield) as a yellow solid. LCMS m/z=467.1 [M+H]⁺; 1H NMR (400 MHz, MeOD-d₄) δ=8.00 (d, J=5.2 Hz, 1H), 7.91 (d, J=1.6 Hz, 1H), 7.78 (s, 1H), 7.72 (s, 1H), 6.84 (d, J=4.8 Hz, 1H), 6.60 (d, J=1.6 Hz, 1H), 4.67-4.60 (m, 1H), 4.21-4.15 (m, 1H), 4.14-4.09 (m, 1H), 4.03-3.97 (m, 1H), 3.89-3.81 (m, 2H), 3.70-3.60 (m, 1H), 3.14-3.06 (m, 1H), 2.76-2.66 (m, 2H), 1.45 (d, J=6.8 Hz, 3H), 1.22-1.17 (m, 1H), 0.84-0.77 (m, 4H).

### Example 329 and 330

### (3R,4S)-1-(6-(1-((1s,3S)-3-cyanocyclobutyl)-1H-pyrazol-4-yl)pyrrolo[1,2-b]pyridazin-4-yl)-3-cyclopropyl-4-methyl-2-oxopyrrolidine-3-carbonitrile and (3R,4S)-1-(6-(1-((1r,3R)-3-cyanocyclobutyl)-1H-pyrazol-4-yl)pyrrolo[1,2-b]pyridazin-4-yl)-3-cyclopropyl-4-methyl-2-oxopyrrolidine-3-carbonitrile

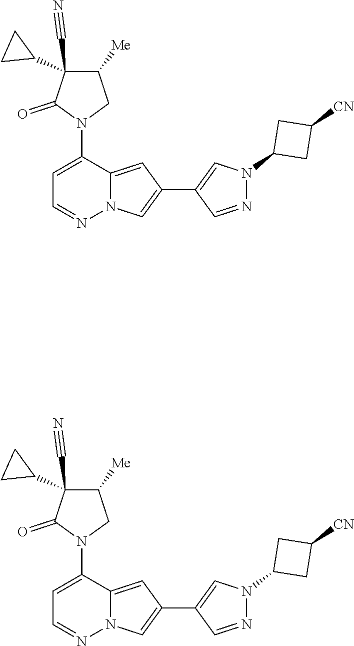 [SMILES: C[C@@H]1CN(c2ccnn3cc(-c4cnn([C@H]5C[C@@H](C#N)C5)c4)cc23)C(=O)[C@]1(C#N)C1CC1 ; C[C@@H]1CN(c2ccnn3cc(-c4cnn([C@H]5C[C@H](C#N)C5)c4)cc23)C(=O)[C@]1(C#N)C1CC1]

Part A: (3R,4S)-1-(6-(1-(3-cyanocyclobutyl)-1H-pyrazol-4-yl)pyrrolo[1,2-b]pyridazin-4-yl)-3-cyclopropyl-4-methyl-2-oxopyrrolidine-3-carbonitrile was obtained as a yellow solid, 100 mg, 44.6% yield from 3-(4-bromo-1H-pyrazol-1-yl)cyclobutane-1-carbonitrile (Preparation 213) and (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69) following the procedure described in Example 319. LCMS m/z=426.2 [M+H]⁺

Part B: (3R,4S)-1-(6-(1-(3-Cyanocyclobutyl)-1H-pyrazol-4-yl)pyrrolo[1,2-b]pyridazin-4-yl)-3-cyclopropyl-4-methyl-2-oxopyrrolidine-3-carbonitrile was further purified by SFC (Column: DAICEL CHIRALPAK AD (250 mm×30 mm, 10 μm), Condition: 50% EtOH (0.1% NH₃H₂O) Flow Rate (mL/min) 80 to give Example 329, (3R,4S)-1-(6-(1-((1s,3S)-3-cyanocyclobutyl)-1H-pyrazol-4-yl)pyrrolo[1,2-b]pyridazin-4-yl)-3-cyclopropyl-4-methyl-2-oxopyrrolidine-3-carbonitrile (10.1 mg, 9.81% yield) as a yellow solid. Relative stereochemistry was determined by 2D nmr. LCMS m/z=426.2 [M+H]⁺; ¹H NMR (500 MHz, DMSO-d₆) δ=8.28 (s, 1H), 8.21 (d, J=2.0 Hz, 1H), 8.14 (d, J=5.5 Hz, 1H), 7.95 (s, 1H), 6.93 (d, J=5.0 Hz, 1H), 6.90 (d, J=2.0 Hz, 1H), 4.93-4.86 (m, 1H), 4.13-4.08 (m, 1H), 4.05-4.00 (m, 1H), 3.27-3.23 (m, 1H), 3.17-3.12 (m, 1H), 2.90-2.80 (m, 4H), 1.53-1.47 (m, 1H), 1.32 (d, J=7.0 Hz, 3H), 0.77-0.68 (m, 2H), 0.60-0.51 (m, 2H).

and Example 330, (3R,4S)-1-(6-(1-((1r,3R)-3-cyanocyclobutyl)-1H-pyrazol-4-yl)pyrrolo[1,2-b]pyridazin-4-yl)-3-cyclopropyl-4-methyl-2-oxopyrrolidine-3-carbonitrile (35.3 mg, 34.5% yield) as a yellow solid. Relative stereochemistry was determined by 2D nmr. LCMS m/z=426.2 [M+H]⁺; ¹H NMR (500 MHz, MeOD-d₄) δ=8.01-7.88 (m, 3H), 7.80 (s, 1H), 6.76 (d, J=5.0 Hz, 1H), 6.71 (d, J=1.5 Hz, 1H), 5.14-5.06 (m, 1H), 4.03 (dd, J=7.5, 9.5 Hz, 1H), 3.89 (t, J=9.5 Hz, 1H), 3.37-3.30 (m, 1H), 3.09-3.01 (m, 1H), 2.96-2.89 (m, 2H), 2.81-2.74 (m, 2H), 1.34-1.28 (m, 4H), 0.73-0.59 (m, 4H).

### Example 331

### (3R,4S)-3-cyclopropyl-4-methyl-1-(6-(1-(1-methylpiperidin-4-yl)-1H-pyrazol-4-yl)pyrrolo[1,2-b]pyridazin-4-yl)-2-oxopyrrolidine-3-carbonitrile

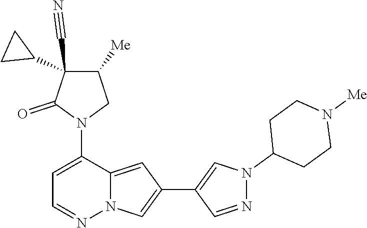 [SMILES: C[C@@H]1CN(c2ccnn3cc(-c4cnn(C5CCN(C)CC5)c4)cc23)C(=O)[C@]1(C#N)C1CC1]

N₂ was bubbled through a mixture of 4-(4-bromo-1H-pyrazol-1-yl)-1-methylpiperidine (145 mg, 0.594 mmol), (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 120 mg, 0.295 mmol), K₂CO₃ (122.46 mg, 0.886 mmol) and Pd(dppf)Cl₂ (24.12 mg, 0.030 mmol) in dioxane (5 mL) and water (1 mL) and the reaction was stirred at 90° C. for 2 h. The reaction mixture was concentrated in vacuum to give the crude, which was purified by Prep-HPLC-3 (43 to 63% gradient) to give (3R,4S)-3-cyclopropyl-4-methyl-1-(6-(1-(1-methylpiperidin-4-yl)-1H-pyrazol-4-yl)pyrrolo[1,2-b]pyridazin-4-yl)-2-oxopyrrolidine-3-carbonitrile (54.4 mg, 41.5% yield) as an orange solid. LCMS m/z=444.2 [M+H]^{+ 1}H NMR (400 MHz, MeOD-d₄) δ=8.08-7.96 (m, 3H), 7.81 (s, 1H), 6.86-6.77 (m, 2H), 4.21-4.14 (m, 1H), 4.13-3.95 (m, 2H), 3.17-3.10 (m, 1H), 3.00 (d, J=12.0 Hz, 2H), 2.33 (s, 3H), 2.24 (t, J=12.0 Hz, 2H), 2.17-2.04 (m, 4H), 1.47-1.34 (m, 4H), 0.81-0.66 (m, 4H).

### Example 332

### (3R,4S)-3-cyclopropyl-4-methyl-1-(6-(1-(1-methylpyrrolidin-3-yl)-1H-pyrazol-4-yl)pyrrolo[1,2-b]pyridazin-4-yl)-2-oxopyrrolidine-3-carbonitrile

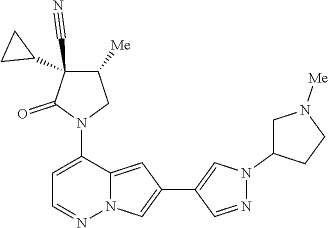 [SMILES: C[C@@H]1CN(c2ccnn3cc(-c4cnn(C5CCN(C)C5)c4)cc23)C(=O)[C@]1(C#N)C1CC1]

To a solution of 4-bromo-1-(1-methylpyrrolidin-3-yl)-1H-pyrazole (Preparation 270, 100 mg, 0.246 mmol) and (3R,4S)-3-cyclopropyl-4-methyl-2-oxo-1-(6-(4,4,5,5-tetramethyl-1,3,2-dioxaborolan-2-yl)pyrrolo[1,2-b]pyridazin-4-yl)pyrrolidine-3-carbonitrile (Preparation 69, 67.96 mg, 0.295 mmol) in dioxane (2 mL) and water (0.5 mL) was added K₂CO₃ (102.05 mg, 0.738 mmol) and Pd(dppf)Cl₂:DCM (80.40 mg, 0.098 mmol) and the reaction was stirred under N₂ at 90° C. for 16 h. The mixture was purified by prep-HPLC-3 (33 to 63% gradient) to give (3R,4S)-3-cyclopropyl-4-methyl-1-(6-(1-(1-methylpyrrolidin-3-yl)-1H-pyrazol-4-yl)pyrrolo[1,2-b]pyridazin-4-yl)-2-oxopyrrolidine-3-carbonitrile (20 mg, 18.9% yield) as a brown solid. LCMS m/z=430.3 [M+H]⁺; ¹H NMR (400 MHz, CDCl₃) δ=7.98 (d, J=5.2 Hz, 1H), 7.91 (d, J=1.2 Hz, 1H), 7.82 (s, 1H), 7.73 (s, 1H), 6.85 (d, J=5.2 Hz, 1H), 6.61 (d, J=1.2 Hz, 1H), 4.92-5.01 (m, 1H), 4.14-4.10 (m, 1H), 3.88 (t, J=10.0 Hz, 1H), 3.13-3.08 (m, 1H), 3.05-2.93 (m, 3H), 2.68-2.60 (m, 1H), 2.59-2.51 (m, 1H), 2.49 (s, 3H), 2.29-2.18 (m, 1H), 1.45 (d, J=7.2 Hz, 3H), 1.26-1.10 (m, 1H), 0.88-0.70 (m, 4H).

### Example 333

### (3R,4S)-3-cyclopropyl-4-methyl-1-(6-(4-methyl-1H-pyrazol-1-yl)pyrrolo[1,2-b]pyridazin-4-yl)-2-oxopyrrolidine-3-carbonitrile

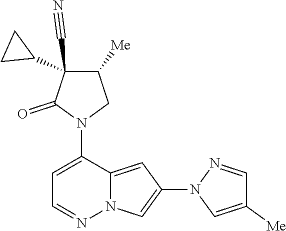 [SMILES: Cc1cnn(-c2cc3c(N4C[C@@H](C)[C@@](C#N)(C5CC5)C4=O)ccnn3c2)c1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 80 mg, 0.223 mmol) in dioxane (2 mL) was added K₂CO₃ (92.34 mg, 0.668 mmol) and 4-methyl-1H-pyrazole (36.57 mg, 0.445 mmol) at 20° C. DMEDA (29.45 mg, 0.334 mmol) and CuI (63.62 mg, 0.334 mmol) were added and the reaction was stirred at 100° C. under N₂ for 12 h. The mixture was poured into water (20 mL) and extracted with DCM (20 mL×3). The combined organic layers were washed with brine (50 mL), dried over Na₂SO₄, filtered and concentrated under vacuum. The crude product was purified by Prep-HPLC-3 (45 to 65% gradient) to give (3R,4S)-3-cyclopropyl-4-methyl-1-(6-(4-methyl-1H-pyrazol-1-yl)pyrrolo[1,2-b]pyridazin-4-yl)-2-oxopyrrolidine-3-carbonitrile (34.1 mg, 42.5% yield) as a yellow solid. LCMS m/z=361.1 [M+H]⁺; ¹H NMR (400 MHz, CDCl₃) δ=8.06 (d, J=5.2 Hz, 1H), 8.01 (d, J=1.6 Hz, 1H), 7.65 (s, 1H), 7.53 (s, 1H), 7.02 (d, J=5.6 Hz, 1H), 6.80 (d, J=1.2 Hz, 1H), 4.16-4.10 (m, 1H), 3.93 (t, J=9.8 Hz, 1H), 3.12-3.04 (m, 1H), 2.18 (s, 3H), 1.43 (d, J=7.2 Hz, 3H), 1.21-1.13 (m, 1H), 0.82-0.75 (m, 4H).

### Example 334

### (3R,4S)-3-cyclopropyl-4-methyl-1-(6-(3-methyl-1H-pyrazol-1-yl)pyrrolo[1,2-b]pyridazin-4-yl)-2-oxopyrrolidine-3-carbonitrile

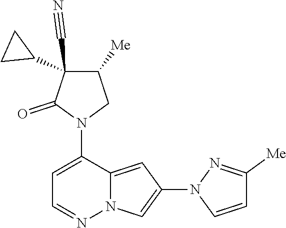 [SMILES: Cc1ccn(-c2cc3c(N4C[C@@H](C)[C@@](C#N)(C5CC5)C4=O)ccnn3c2)n1]

(3R,4S)-3-Cyclopropyl-4-methyl-1-(6-(3-methyl-1H-pyrazol-1-yl)pyrrolo[1,2-b]pyridazin-4-yl)-2-oxopyrrolidine-3-carbonitrile was obtained, 25.1 mg, 31.3% yield as a white solid, from 3-methyl-1H-pyrazole and (3R,4S)-1-(6-bromopyrrolo[1,2-b]pyridazin-4-yl)-3-cyclopropyl-4-methyl-2-oxopyrrolidine-3-carbonitrile (Preparation 68) following the procedure described in Example 333. LCMS m/z=361.1 [M+H]⁺; ¹H NMR (500 MHz, DMSO-d₆) δ=8.33 (d, J=2.0 Hz, 1H), 8.29 (d, J=2.5 Hz, 1H), 8.24 (d, J=5.0 Hz, 1H), 7.04-7.02 (m, 2H), 6.32 (d, J=2.0 Hz, 1H), 4.09-4.06 (m, 2H), 3.19-3.14 (m, 1H), 2.28 (s, 3H), 1.55-1.52 (m, 1H), 1.31 (d, J=7.0 Hz, 3H), 0.70-0.72 (m, 1H), 0.70-0.68 (m, 1H), 0.56-0.53 (m, 2H).

### Example 335

### (3R,4S)-1-(6-(1H-pyrazol-1-yl)pyrrolo[1,2-b]pyridazin-4-yl)-3-cyclopropyl-4-methyl-2-oxopyrrolidine-3-carbonitrile

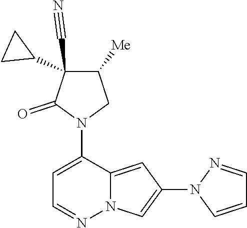 [SMILES: C[C@@H]1CN(c2ccnn3cc(-n4cccn4)cc23)C(=O)[C@]1(C#N)C1CC1]

(3R,4S)-1-(6-(1H-Pyrazol-1-yl)pyrrolo[1,2-b]pyridazin-4-yl)-3-cyclopropyl-4-methyl-2-oxopyrrolidine-3-carbonitrile was obtained as a yellow solid, 25.6 mg, 33.2% yield, from (3R,4S)-1-(6-bromopyrrolo[1,2-b]pyridazin-4-yl)-3-cyclopropyl-4-methyl-2-oxopyrrolidine-3-carbonitrile (Preparation 68) and pyrazole, following a similar procedure to that described in Example 333. LCMS m/z=347.1 [M+H]⁺; 1H NMR (400 MHz, CDCl₃) δ=8.15-8.05 (m, 2H), 7.88 (d, J=2.0 Hz, 1H), 7.73 (d, J=1.2 Hz, 1H), 7.03 (d, J=5.6 Hz, 1H), 6.84 (d, J=2.0 Hz, 1H), 6.48 (t, J=2.0 Hz, 1H), 4.17-4.12 (m, 1H), 3.93 (t, J=9.8 Hz, 1H), 3.14-3.04 (m, 1H), 1.44 (d, J=7.2 Hz, 3H), 1.22-1.14 (m, 1H), 0.83-0.76 (m, 4H).

### Example 336

### (3R,4S)-1-(6-(1H-imidazol-1-yl)pyrrolo[1,2-b]pyridazin-4-yl)-3-cyclopropyl-4-methyl-2-oxopyrrolidine-3-carbonitrile

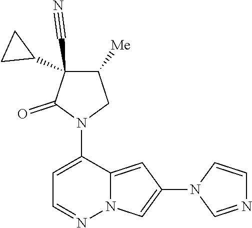 [SMILES: C[C@@H]1CN(c2ccnn3cc(-n4ccnc4)cc23)C(=O)[C@]1(C#N)C1CC1]

(3R,4S)-1-(6-(1H-Imidazol-1-yl)pyrrolo[1,2-b]pyridazin-4-yl)-3-cyclopropyl-4-methyl-2-oxopyrrolidine-3-carbonitrile was obtained as a white solid, 7.1 mg, 4.9% yield, from (3R,4S)-1-(6-bromopyrrolo[1,2-b]pyridazin-4-yl)-3-cyclopropyl-4-methyl-2-oxopyrrolidine-3-carbonitrile (Preparation 68) and imidazole following the procedure described in Example 333. LCMS m/z=347.1 [M+H]⁺; ¹H NMR (500 MHz, MeOD-d₄) δ=8.25-8.23 (m, 2H), 8.18 (d, J=5.5 Hz, 1H), 7.65 (s, 1H), 7.16 (s, 1H), 7.04 (d, J=5.0 Hz, 1H), 6.98 (d, J=1.5 Hz, 1H), 4.18-4.14 (m, 1H), 4.06-4.01 (m, 1H), 3.21-3.16 (m, 1H), 1.43 (d, J=7.0 Hz, 4H), 0.82-0.80 (m, 1H), 0.78-0.74 (m, 1H), 0.72-0.70 (m, 2H).

### Example 337

### (3R,4S)-3-cyclopropyl-1-(6-(4-(methoxymethyl)-1H-pyrazol-1-yl)pyrrolo[1,2-b]pyridazin-4-yl)-4-methyl-2-oxopyrrolidine-3-carbonitrile

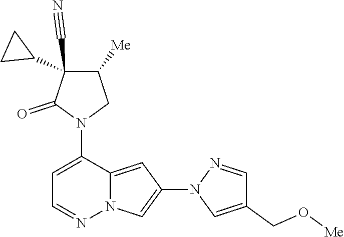 [SMILES: COCc1cnn(-c2cc3c(N4C[C@@H](C)[C@@](C#N)(C5CC5)C4=O)ccnn3c2)c1]

(3R,4S)-3-Cyclopropyl-1-(6-(4-(methoxymethyl)-1H-pyrazol-1-yl)pyrrolo[1,2-b]pyridazin-4-yl)-4-methyl-2-oxopyrrolidine-3-carbonitrile was obtained as a light yellow solid, 30 mg, 34.5% yield, from (3R,4S)-1-(6-bromopyrrolo[1,2-b]pyridazin-4-yl)-3-cyclopropyl-4-methyl-2-oxopyrrolidine-3-carbonitrile (Preparation 68) and 4-(methoxymethyl)-1H-pyrazole, following a similar procedure to that described in Example 333, except the crude product was purified by prep-HPLC-2 (40 to 70% gradient). LCMS m/z=391.1 [M+H]⁺; ¹H NMR (500 MHz, DMSO-d₆) δ=8.44 (s, 1H), 8.37 (d, J=2.0 Hz, 1H), 8.26 (d, J=5.0 Hz, 1H), 7.69 (s, 1H), 7.09-7.05 (m, 2H), 4.38 (s, 2H), 4.11-4.06 (m, 2H), 3.29 (s, 3H), 3.19-3.14 (m, 1H), 1.55-1.52 (m, 1H), 1.32 (d, J=6.5 Hz, 3H), 0.75-0.68 (m, 2H), 0.57-0.53 (m, 2H).

### Example 338

### (3R,4S)-3-cyclopropyl-1-(6-(4-(difluoromethyl)-1H-pyrazol-1-yl)pyrrolo[1,2-b]pyridazin-4-yl)-4-methyl-2-oxopyrrolidine-3-carbonitrile

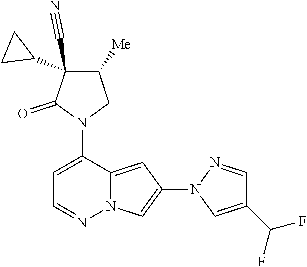 [SMILES: C[C@@H]1CN(c2ccnn3cc(-n4cc(C(F)F)cn4)cc23)C(=O)[C@]1(C#N)C1CC1]

(3R,4S)-3-Cyclopropyl-1-(6-(4-(difluoromethyl)-1H-pyrazol-1-yl)pyrrolo[1,2-b]pyridazin-4-yl)-4-methyl-2-oxopyrrolidine-3-carbonitrile was obtained as a light yellow solid, 7.5 mg, 6.8% yield, from (3R,4S)-1-(6-bromopyrrolo[1,2-b]pyridazin-4-yl)-3-cyclopropyl-4-methyl-2-oxopyrrolidine-3-carbonitrile (Preparation 68) and 4-(difluoromethyl)-1H-pyrazole, following a similar procedure to that described in Example 333, except the crude product was purified by Prep-HPLC-21 (gradient: 47 to 77%). LCMS m/z=397.1 [M+H]⁺; ¹H NMR (500 MHz, MeOD-d₄) δ=8.41 (s, 1H), 8.13 (s, 1H), 8.05 (d, J=5.5 Hz, 1H), 7.77 (s, 1H), 6.92-6.80 (m, 3H), 4.06-4.02 (m, 1H), 3.93-3.90 (m, 1H), 3.09-3.03 (m, 1H), 1.33-1.30 (m, 4H), 0.71-0.64 (m, 2H), 0.62-0.59 (m, 2H).

### Example 339

### (3R,4S)-1-(6-(4-(cyanomethyl)-1H-pyrazol-1-yl)pyrrolo[1,2-b]pyridazin-4-yl)-3-cyclopropyl-4-methyl-2-oxopyrrolidine-3-carbonitrile

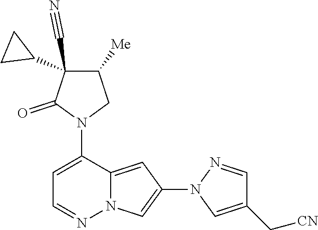 [SMILES: C[C@@H]1CN(c2ccnn3cc(-n4cc(CC#N)cn4)cc23)C(=O)[C@]1(C#N)C1CC1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 80.0 mg, 0.223 mmol) in toluene (2 mL) was added CuI (21.21 mg, 0.111 mmol) and 4-(cyanomethyl)-1H-pyrazole (28.63 mg, 0.267 mmol) at 20° C. (1R, 2R)-N1,N2-dimethylcyclohexane-1,2-diamine (31.68 mg, 0.223 mmol) and K₂CO₃ (61.56 mg, 0.445 mmol) were added and the reaction was stirred at 100° C. under N₂ for 16 h. The mixture was poured into water (10 mL) and extracted with DCM (10 mL×3). The combined organic layers were washed with brine (50 mL), dried over Na₂SO₄, filtered and concentrated under vacuum. The crude product was purified by Prep-HPLC-3 (35 to 55% gradient) to give (3R,4S)-1-(6-(4-(cyanomethyl)-1H-pyrazol-1-yl)pyrrolo[1,2-b]pyridazin-4-yl)-3-cyclopropyl-4-methyl-2-oxopyrrolidine-3-carbonitrile (18.2 mg, 21.2% yield) as a yellow solid. LCMS m/z=386.3 [M+H]⁺; ¹H NMR (400 MHz, CDCl₃) δ=8.12-8.07 (m, 2H), 7.94 (s, 1H), 7.68 (s, 1H), 7.05 (d, J=5.2 Hz, 1H), 6.81 (d, J=1.8 Hz, 1H), 4.16 (m, 1H), 3.94 (t, J=9.8 Hz, 1H), 3.72 (s, 2H), 3.18-3.06 (m, 1H), 1.47 (d, J=6.8 Hz, 3H), 1.24-1.15 (m, 1H), 0.85-0.80 (m, 3H).

### Example 340

### (3R,4S)-3-cyclopropyl-4-methyl-2-oxo-1-(6-(2-oxopyridin-1(2H)-yl)pyrrolo[1,2-b]pyridazin-4-yl)pyrrolidine-3-carbonitrile

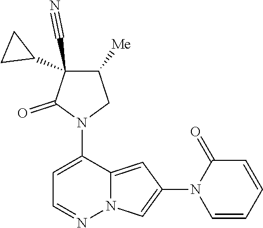 [SMILES: C[C@@H]1CN(c2ccnn3cc(-n4ccccc4=O)cc23)C(=O)[C@]1(C#N)C1CC1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 200 mg, 0.557 mmol) in DMSO (8 mL) was added K₂CO₃ (153.9 mg, 1.11 mmol) and pyridin-2(1H)-one (63.54 mg, 0.668 mmol) at 20° C. 4,7-Dimethoxy-1,10-phenanthroline (20.06 mg, 0.084 mmol) and CuI (10.60 mg, 0.056 mmol) were added and the reaction was stirred at 110° C. under N₂ for 16 h. The mixture was poured into water (20 mL) and extracted with DCM (20 mL×3). The combined organic layers were washed with brine (50 mL), dried over Na₂SO₄, filtered and concentrated under vacuum. The crude product was purified by Prep-HPLC-3 (33 to 53% gradient) to give (3R,4S)-3-cyclopropyl-4-methyl-2-oxo-1-(6-(2-oxopyridin-1(2H)-yl)pyrrolo[1,2-b]pyridazin-4-yl)pyrrolidine-3-carbonitrile (20 mg, 9.5% yield) as a yellow solid. LCMS m/z=374.1 [M+H]⁺; ¹H NMR (400 MHz, CDCl₃) δ: =8.17 (s, 1H), 8.09 (d, J=4.8 Hz, 1H), 7.57 (d, J=6.4 Hz, 1H), 7.40 (t, J=7.0 Hz, 1H), 6.94 (d, J=5.2 Hz, 1H), 6.83 (s, 1H), 6.70 (d, J=9.2 Hz, 1H), 6.30 (t, J=6.6 Hz, 1H), 4.12 (t, J=9.0 Hz, 1H), 3.86 (t, J=9.6 Hz, 1H), 3.13-3.04 (m, 1H), 1.42 (d, J=6.8 Hz, 3H), 1.20-1.12 (m, 1H), 0.83-0.74 (m, 4H).

### Example 341

### (3R,4S)-3-cyclopropyl-4-methyl-1-(6-(4-methyl-1H-imidazol-1-yl)pyrrolo[1,2-b]pyridazin-4-yl)-2-oxopyrrolidine-3-carbonitrile

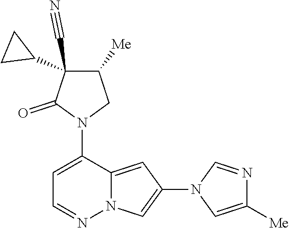 [SMILES: Cc1cn(-c2cc3c(N4C[C@@H](C)[C@@](C#N)(C5CC5)C4=O)ccnn3c2)cn1]

To a mixture of (3R,4S)-1-(6-bromopyrrolo[1,2-b]pyridazin-4-yl)-3-cyclopropyl-4-methyl-2-oxopyrrolidine-3-carbonitrile (Preparation 68, 100 mg, 0.278 mmol), 4-methyl-1H-imidazole (45.71 mg, 0.557 mmol) and K₂CO₃ (76.95 mg, 0.557 mmol) in dioxane (2 mL) and toluene (2 mL) was added Pd₂(dba)₃ (127.5 mg, 0.139 mmol) and Me₄t-BuXPhos (66.92 mg, 0.139 mmol) at 25° C. The reaction mixture was concentrated to give a crude, which was purified by prep-HPLC-2 (36 to 68% gradient) to give (3R,4S)-3-cyclopropyl-4-methyl-1-(6-(4-methyl-1H-imidazol-1-yl)pyrrolo[1,2-b]pyridazin-4-yl)-2-oxopyrrolidine-3-carbonitrile (53.4 mg, 53.2% yield) as a white solid. LCMS m/z=361.1 [M+H]⁺; ¹H NMR: (500 MHz, DMSO-d₆) δ=8.43 (d, J=2.0 Hz, 1H), 8.25 (d, J=5.5 Hz, 1H), 8.18 (d, J=1.5 Hz, 1H), 7.47 (d, J=1.0 Hz, 1H), 7.07-7.06 (m, 2H), 4.10-4.03 (m, 2H), 3.17-3.12 (m, 1H), 2.17 (s, 3H), 1.50-1.47 (m, 1H), 1.31 (d, J=7.0 Hz, 3H), 0.75-0.69 (m, 2H), 0.57-0.53 (m, 2H).

### Example 342

### (3R,4S)-3-cyclopropyl-4-methyl-1-(6-(3-methyl-1H-1,2,4-triazol-1-yl)pyrrolo[1,2-b]pyridazin-4-yl)-2-oxopyrrolidine-3-carbonitrile

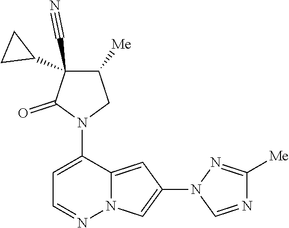 [SMILES: Cc1ncn(-c2cc3c(N4C[C@@H](C)[C@@](C#N)(C5CC5)C4=O)ccnn3c2)n1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 200 mg, 0.557 mmol) in dioxane (5 mL) and toluene (5 mL) was added K₂CO₃ (230.9 mg, 1.67 mmol) and 3-methyl-1H-1,2,4-triazole (138.8 mg, 1.67 mmol) at 20° C. Me₄t-BuXPhos (53.53 mg, 0.111 mmol) and Pd₂(dba)₃ (50.98 mg, 0.056 mmol) was added and the reaction was stirred at 120° C. under N₂ for 24 h. The mixture was poured into water (20 mL) and extracted with DCM (20 mL×3). The combined organic layers were washed with brine (50 mL), dried over Na₂SO₄, filtered and concentrated under vacuum. The crude was purified by Prep-HPLC-23 (gradient: 30 to 50%) to give (3R,4S)-3-cyclopropyl-4-methyl-1-(6-(3-methyl-1H-1,2,4-triazol-1-yl)pyrrolo[1,2-b]pyridazin-4-yl)-2-oxopyrrolidine-3-carbonitrile (8.8 mg, 4.4% yield) as a yellow solid. LCMS m/z=362.2 [M+H]⁺; ¹H NMR (400 MHz, CDCl₃) δ: =8.41 (s, 1H), 8.12-8.07 (m, 2H), 7.02 (d, J=5.2 Hz, 1H), 6.80 (d, J=2.0 Hz, 1H), 4.19-4.10 (m, 1H), 3.92 (t, J=9.8 Hz, 1H), 3.16-3.06 (m, 1H), 2.53 (s, 3H), 1.46 (d, J=6.8 Hz, 3H), 1.23-1.14 (m, 1H), 0.83-0.78 (m, 4H).

### Example 343

### (3R,4S)-3-cyclopropyl-4-methyl-1-(6-(4-methyl-2H-1,2,3-triazol-2-yl)pyrrolo[1,2-b]pyridazin-4-yl)-2-oxopyrrolidine-3-carbonitrile

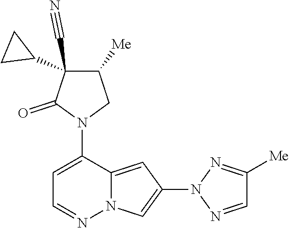 [SMILES: Cc1cnn(-c2cc3c(N4C[C@@H](C)[C@@](C#N)(C5CC5)C4=O)ccnn3c2)n1]

To a solution of (3R,4S)-1-(6-bromopyrrolo[1,2-b]pyridazin-4-yl)-3-cyclopropyl-4-methyl-2-oxopyrrolidine-3-carbonitrile (Preparation 68, 250 mg, 0.696 mmol) in dioxane (10 mL) and toluene (10 mL) was added K₂CO₃ (288.6 mg, 2.09 mmol) and 4-methyl-2H-1,2,3-triazole (115.7 mg, 1.39 mmol). Me₄tBu-XPhos (334.6 mg, 0.696 mmol) and Pd₂(dba)₃ (637.3 mg, 0.696 mmol) were added and the reaction was stirred at 100° C. under N₂ for 16 h. The mixture was poured into water (50 mL) and extracted with DCM (50 mL×3). The combined organic layers were washed with brine (100 mL), dried over Na₂SO₄, filtered and concentrated under vacuum. The crude product was purified by Prep-HPLC-18 (gradient: 53 to 73%). The product was further purified by SFC (column: DAICEL CHIRALCEL OJ (250 mm×30 mm, 10 μm), 30% EtOH (0.1% NH₃·H₂O), as mobile phase, Flow Rate (mL/min): 70) to give (3R,4S)-3-cyclopropyl-4-methyl-1-(6-(4-methyl-2H-1,2,3-triazol-2-yl)pyrrolo[1,2-b]pyridazin-4-yl)-2-oxopyrrolidine-3-carbonitrile (16.8 mg, 6.6% yield) as a yellow solid. LCMS m/z=362.2 [M+H]⁺; ¹H NMR (400 MHz, CDCl₃) δ: =8.19 (d, J=2.0 Hz, 1H), 8.01 (d, J=5.2 Hz, 1H), 7.49 (s, 1H), 7.04 (d, J=5.2 Hz, 1H), 6.90 (d, J=2.0 Hz, 1H), 4.14-4.07 (m, 1H), 3.89 (t, J=9.6 Hz, 1H), 3.05-2.95 (m, 1H), 2.36 (s, 3H), 1.36 (d, J=6.8 Hz, 3H), 1.14-1.06 (m, 1H), 0.77-0.68 (m, 4H).

### Example 344

### (3R,4S)-1-(2-(1-(bicyclo[1.1.1]pentan-1-yl)-1H-pyrazol-4-yl)furo[3,2-b]pyridin-7-yl)-3-cyclopropyl-4-methyl-2-oxopyrrolidine-3-carbonitrile

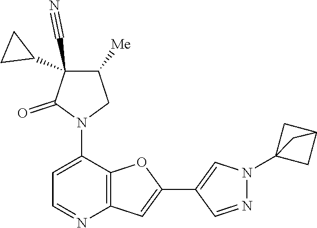 [SMILES: C[C@@H]1CN(c2ccnc3cc(-c4cnn(C56CC(C5)C6)c4)oc23)C(=O)[C@]1(C#N)C1CC1]

To a solution of 2-(1-(bicyclo[1.1.1]pentan-1-yl)-1H-pyrazol-4-yl)-7-chlorofuro[3,2-b]pyridine (Preparation 255, 35 mg, 0.122 mmol) in dioxane (1 mL) was added K₂CO₃ (50.79 mg, 0.367 mmol) and Pd₂(dba)₃ (33.65 mg, 0.037 mmol) at 20° C. XantPhos (21.26 mg, 0.037 mmol) and (3R,4S)-3-cyclopropyl-4-methyl-2-oxopyrrolidine-3-carbonitrile (Preparation 21, 36.21 mg, 0.220 mmol) was added and the reaction was stirred at 100° C. under N₂ for 16 h. The mixture was concentrated under vacuum to give the crude, which was purified by Prep-HPLC-2 (43 to 73% gradient) to give (3R,4S)-1-(2-(1-(bicyclo[1.1.1]pentan-1-yl)-1H-pyrazol-4-yl)furo[3,2-b]pyridin-7-yl)-3-cyclopropyl-4-methyl-2-oxopyrrolidine-3-carbonitrile (4.1 mg, 8.10% yield) as a yellow solid. LCMS m/z=414.1 [M+H]⁺; ¹H NMR (400 MHz, CDCl₃) δ=8.46 (d, J=5.6 Hz, 1H), 7.89 (s, 1H), 7.83-7.76 (m, 2H), 6.91 (s, 1H), 4.40 (dd, J₁=7.6 Hz, J₂=10.4 Hz, 1H), 3.99 (t, J=10.0 Hz, 1H), 3.17-3.10 (m, 1H), 2.70 (s, 1H), 2.40-2.37 (m, 6H), 1.46 (d, J=6.8 Hz, 3H), 1.22-1.15 (m, 1H), 0.87-0.75 (m, 4H).

### Example 345

### (3R,4S)-3-cyclopropyl-4-methyl-1-(2-(4-methyl-1H-pyrazol-1-yl)furo[3,2-b]pyridin-7-yl)-2-oxopyrrolidine-3-carbonitrile

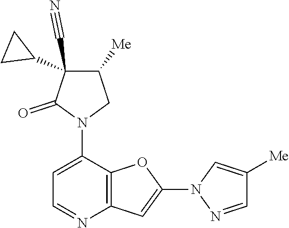 [SMILES: Cc1cnn(-c2cc3nccc(N4C[C@@H](C)[C@@](C#N)(C5CC5)C4=O)c3o2)c1]

To a mixture of 7-chloro-2-(4-methyl-1H-pyrazol-1-yl)furo[3,2-b]pyridine (Preparation 258, 25 mg, 0.107 mmol), (3R,4S)-3-cyclopropyl-4-methyl-2-oxopyrrolidine-3-carbonitrile (Preparation 21, 21.08 mg, 0.128 mmol) and K₂CO₃ (44.36 mg, 0.321 mmol) in dioxane (1 mL) was added Pd₂(dba)₃ (9.80 mg, 0.0107 mmol) and Xantphos (12.38 mg, 0.021 mmol) at 25° C. The mixture was stirred at 100° C. for 16 h. The reaction mixture was concentrated in vacuo and the crude was purified by prep-HPLC-3 (35 to 64% gradient) to give (3R,4S)-3-cyclopropyl-4-methyl-1-(2-(4-methyl-1H-pyrazol-1-yl)furo[3,2-b]pyridin-7-yl)-2-oxopyrrolidine-3-carbonitrile (8.7 mg, 22.5% yield) as a white solid. LCMS m/z=362.1 [M+H]⁺; ¹H NMR (500 MHz, MeOD-d₄) δ=8.42 (s, 1H), 8.15 (d, J=3.0 Hz, 1H), 7.91 (s, 1H), 7.74 (d, J=3.5 Hz, 1H), 6.88 (d, J=3.5 Hz, 1H), 4.47-4.43 (m, 1H), 4.17-4.12 (m, 1H), 3.20-3.16 (m, 1H), 2.21 (d, J=3.5 Hz, 3H), 1.47-1.44 (m, 3H), 1.44-1.42 (m, 1H), 0.82-0.81 (m, 1H), 0.75-0.71 (m, 3H).

### Example 346

### (3R,4S)-3-cyclopropyl-1-(2-(1-isopropyl-1H-pyrazol-4-yl)furo[3,2-b]pyridin-7-yl)-4-methyl-2-oxopyrrolidine-3-carbonitrile

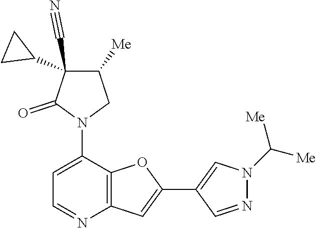 [SMILES: CC(C)n1cc(-c2cc3nccc(N4C[C@@H](C)[C@@](C#N)(C5CC5)C4=O)c3o2)cn1]

To a solution of 7-chloro-2-(1-isopropyl-1H-pyrazol-4-yl)furo[3,2-b]pyridine (Preparation 256, 100 mg, 0.382 mmol) in dioxane (4 mL) was added K₂CO₃ (158.4 mg, 1.15 mmol) and (3R,4S)-3-cyclopropyl-4-methyl-2-oxopyrrolidine-3-carbonitrile (Preparation 21, 94.12 mg, 0.573 mmol) at 20° C. XantPhos (33.16 mg, 0.057 mmol) and Pd₂(dba)₃ (52.49 mg, 0.57 mmol) were added and the reaction was stirred at 100° C. under N₂ for 16 h. The mixture was poured into water (10 mL) and extracted with DCM (10 mL×3). The combined organic layers were washed with brine (20 mL), dried over Na₂SO₄, filtered and concentrated in vacuo. The crude was purified by Prep-HPLC-2 (43 to 70% gradient) to give (3R,4S)-3-cyclopropyl-1-(2-(1-isopropyl-1H-pyrazol-4-yl)furo[3,2-b]pyridin-7-yl)-4-methyl-2-oxopyrrolidine-3-carbonitrile (41 mg, 27.6% yield) as a yellow solid. LCMS m/z=390.2 [M+H]⁺; ¹H NMR (400 MHz, CDCl₃) δ: =8.45 (d, J=5.6 Hz, 1H), 7.90 (s, 1H), 7.85 (s, 1H), 7.78 (d, J=5.2 Hz, 1H), 6.90 (s, 1H), 4.75-4.49 (m, 1H), 4.45-4.29 (m, 1H), 4.01 (t, J=9.8 Hz, 1H), 3.37-2.91 (m, 1H), 1.60 (d, J=6.8 Hz, 6H), 1.47 (d, J=6.8 Hz, 3H), 1.26-1.16 (m, 1H), 0.87-0.71 (m, 4H).

### Example 347

### (3R,4S)-3-cyclopropyl-1-(2-(1-cyclopropyl-1H-pyrazol-4-yl)furo[3,2-b]pyridin-7-yl)-4-methyl-2-oxopyrrolidine-3-carbonitrile

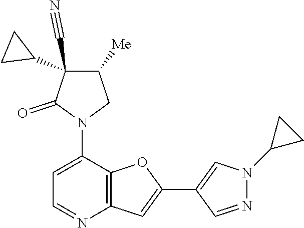 [SMILES: C[C@@H]1CN(c2ccnc3cc(-c4cnn(C5CC5)c4)oc23)C(=O)[C@]1(C#N)C1CC1]

(3R,4S)-3-Cyclopropyl-1-(2-(1-cyclopropyl-1H-pyrazol-4-yl)furo[3,2-b]pyridin-7-yl)-4-methyl-2-oxopyrrolidine-3-carbonitrile was obtained as a yellow solid, 41.4 mg, 25.6% yield, from 7-chloro-2-(1-cyclopropyl-1H-pyrazol-4-yl)furo[3,2-b]pyridine (Preparation 257) and (3R,4S)-3-cyclopropyl-4-methyl-2-oxopyrrolidine-3-carbonitrile (Preparation 21), following the procedure described in Example 346. LCMS m/z=388.1 [M+H]⁺; ¹H NMR (400 MHz, CDCl₃) δ: =8.45 (d, J=5.6 Hz, 1H), 7.86 (d, J=9.2 Hz, 2H), 7.78 (d, J=5.6 Hz, 1H), 6.89 (s, 1H), 4.43-4.33 (m, 1H), 4.00 (t, J=10.0 Hz, 1H), 3.73-3.66 (m, 1H), 3.18-3.08 (m, 1H), 1.47 (d, J=6.8 Hz, 3H), 1.25-1.19 (m, 3H), 1.15-1.09 (m, 2H), 0.86-0.72 (m, 4H).

### Example 348

### (3R,4S)-3-cyclopropyl-4-methyl-1-(5-(1-(oxetan-3-yl)-1H-pyrazol-4-yl)benzo[d]thiazol-7-yl)-2-oxopyrrolidine-3-carbonitrile

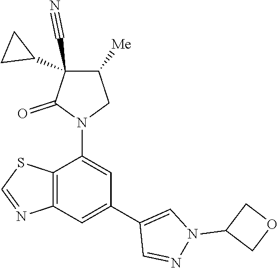 [SMILES: C[C@@H]1CN(c2cc(-c3cnn(C4COC4)c3)cc3ncsc23)C(=O)[C@]1(C#N)C1CC1]

To a mixture of 5-(1-(oxetan-3-yl)-1H-pyrazol-4-yl)benzo[d]thiazol-7-yl trifluoromethanesulfonate (Preparation 253, 50 mg, 0.123 mmol), (3R,4S)-3-cyclopropyl-4-methyl-2-oxopyrrolidine-3-carbonitrile (Preparation 21, 24.30 mg, 0.148 mmol) and K₂CO₃ (51.14 mg, 0.370 mmol) in toluene (2 mL) was added Pd₂(dba)₃ (11.29 mg, 0.0123 mmol) and Xantphos (14.27 mg, 0.0247 mmol) at 20° C. The mixture was stirred at 85° C. for 5 h. The reaction mixture was concentrated in vacuo and the crude was purified by prep-HPLC-3 (Gradient: 25 to 55%) to give (3R,4S)-3-cyclopropyl-4-methyl-1-(5-(1-(oxetan-3-yl)-1H-pyrazol-4-yl)benzo[d]thiazol-7-yl)-2-oxopyrrolidine-3-carbonitrile (14 mg, 27.1% yield) as a white solid. LCMS m/z=420.1 [M+H]⁺; ¹H NMR (500 MHz, MeOD-d₄) δ=9.28 (s, 1H), 8.35 (s, 1H), 8.25 (s, 1H), 8.13 (s, 1H), 7.79 (s, 1H), 5.65-5.61 (m, 1H), 5.10 (d, J=7.0 Hz, 4H), 4.12-4.08 (m, 1H), 3.93-3.89 (m, 1H), 3.26-3.22 (m, 1H), 1.45-1.43 (m, 4H), 0.84-0.79 (m, 2H), 0.77-0.74 (m, 2H).

### Example 349

### (3R,4S)-3-cyclopropyl-1-(5-(1-(difluoromethyl)-1H-pyrazol-4-yl)benzo[d]thiazol-7-yl)-4-methyl-2-oxopyrrolidine-3-carbonitrile

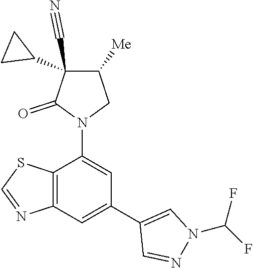 [SMILES: C[C@@H]1CN(c2cc(-c3cnn(C(F)F)c3)cc3ncsc23)C(=O)[C@]1(C#N)C1CC1]

(3R,4S)-3-Cyclopropyl-1-(5-(1-(difluoromethyl)-1H-pyrazol-4-yl)benzo[d]thiazol-7-yl)-4-methyl-2-oxopyrrolidine-3-carbonitrile was obtained as a white solid, 30 mg, 48.3% yield, from 5-(1-(difluoromethyl)-1H-pyrazol-4-yl)benzo[d]thiazol-7-yl trifluoromethanesulfonate (Preparation 254) and (3R,4S)-3-cyclopropyl-4-methyl-2-oxopyrrolidine-3-carbonitrile (Preparation 21) following the procedure described in Example 348. LCMS m/z=414.1 [M+H]⁺; ¹H NMR (500 MHz, DMSO-d₆) δ=9.45 (s, 1H), 8.95 (s, 1H), 8.49-8.48 (m, 2H), 7.96-7.77 (m, 2H), 4.12-4.09 (m, 1H), 3.93-3.89 (m, 1H), 3.22-3.16 (m, 1H), 1.48-1.45 (m, 1H), 1.32 (d, J=6.5 Hz, 3H), 0.77-0.73 (m, 2H), 0.60-0.59 (m, 2H).

### Example 350

### (3R,4S)-3-cyclopropyl-4-methyl-1-(5-(1-methyl-1H-pyrazol-4-yl)benzo[d]thiazol-7-yl)-2-oxopyrrolidine-3-carbonitrile

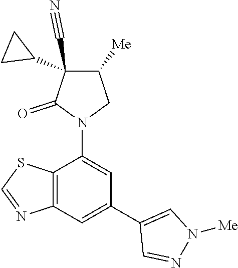 [SMILES: C[C@@H]1CN(c2cc(-c3cnn(C)c3)cc3ncsc23)C(=O)[C@]1(C#N)C1CC1]

3R,4S)-3-cyclopropyl-4-methyl-1-(5-(1-methyl-1H-pyrazol-4-yl)benzo[d]thiazol-7-yl)-2-oxopyrrolidine-3-carbonitrile was obtained as a white solid, 16.6 mg, 40% yield, from 5-(1-methyl-1H-pyrazol-4-yl)benzo[d]thiazol-7-yl trifluoromethanesulfonate (Preparation 252) and (3R,4S)-3-cyclopropyl-4-methyl-2-oxopyrrolidine-3-carbonitrile (Preparation 21) following the procedure described in Example 348. LCMS m/z=378.1 [M+H]⁺; ¹H NMR (400 MHz, MeOD-d₄) δ=9.29 (s, 1H), 8.24 (d, J=1.2 Hz, 1H), 8.16 (s, 1H), 8.00 (s, 1H), 7.77 (d, J=1.2 Hz, 1H), 4.13-4.08 (m, 1H), 3.98 (s, 3H), 3.94-3.89 (m, 1H), 3.25-3.20 (m, 1H), 1.44 (d, J=6.8 Hz, 4H), 0.84-0.80 (m, 2H), 0.78-0.75 (m, 2H).

### Example 351

### (3R,4S)-3-cyclopropyl-4-methyl-1-(6-(1-methyl-1H-pyrazol-4-yl)benzo[c]isothiazol-4-yl)-2-oxopyrrolidine-3-carbonitrile

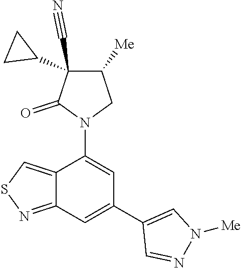 [SMILES: C[C@@H]1CN(c2cc(-c3cnn(C)c3)cc3nscc23)C(=O)[C@]1(C#N)C1CC1]

(3R,4S)-3-Cyclopropyl-4-methyl-1-(6-(1-methyl-1H-pyrazol-4-yl)benzo[c]isothiazol-4-yl)-2-oxopyrrolidine-3-carbonitrile was obtained as a white solid, 31 mg, 29.8% yield, from 6-(1-methyl-1H-pyrazol-4-yl)benzo[c]isothiazol-4-yl trifluoromethanesulfonate (Preparation 263) and (3R,4S)-3-cyclopropyl-4-methyl-2-oxopyrrolidine-3-carbonitrile (Preparation 21), following a similar method to that described in Example 348, except the compound was purified by HPLC-3 (Gradient: 27 to 57%). LCMS m/z=378.1 [M+H]⁺; ¹H NMR (500 MHz, MeOD-d₄) δ=9.45 (s, 1H), 8.19 (s, 1H), 8.03 (s, 1H), 7.95 (s, 1H), 7.58 (s, 1H), 4.09-4.05 (m, 1H), 3.98-3.94 (m, 4H), 3.31-3.27 (m, 1H), 1.52-1.51 (m, 1H), 1.45 (d, J=7.0 Hz, 3H), 0.85-0.80 (m, 2H), 0.78-0.75 (m, 2H).

### Example 352

### (3R,4S)-3-cyclopropyl-4-methyl-1-(6-(1-(oxetan-3-yl)-1H-pyrazol-4-yl)thiazolo[5,4-c]pyridin-4-yl)-2-oxopyrrolidine-3-carbonitrile

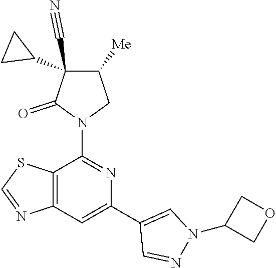 [SMILES: C[C@@H]1CN(c2nc(-c3cnn(C4COC4)c3)cc3ncsc23)C(=O)[C@]1(C#N)C1CC1]

To a solution of (3R,4S)-1-(6-(1H-pyrazol-4-yl)thiazolo[5,4-c]pyridin-4-yl)-3-cyclopropyl-4-methyl-2-oxopyrrolidine-3-carbonitrile (Preparation 248, 20 mg, 0.055 mmol) in DMF was added Cs₂CO₃ (40.05 mg, 0.123 mmol) and 3-iodooxetane (29.99 mg, 0.163 mmol) and the reaction was stirred at 90° C. for 1 h. The mixture was concentrated under vacuum to give the crude, which was purified by Prep-HPLC-20 (gradient: 38 to 48%) to give (3R,4S)-3-cyclopropyl-4-methyl-1-(6-(1-(oxetan-3-yl)-1H-pyrazol-4-yl)thiazolo[5,4-c]pyridin-4-yl)-2-oxopyrrolidine-3-carbonitrile (15.4 mg, 62.6% yield) as a yellow solid. LCMS m/z=421.2 [M+H]⁺; ¹H NMR (400 MHz, CDCl₃) δ=9.20 (s, 1H), 8.18 (s, 1H), 8.13 (s, 1H), 8.06 (s, 1H), 5.55 (t, J=6.8 Hz, 1H), 5.15-5.12 (m, 4H), 4.40-4.34 (m, 1H), 4.05-3.99 (m, 1H), 3.17-3.10 (m, 1H), 1.48 (d, J=6.8 Hz, 3H), 1.27-1.19 (m, 1H), 0.88-0.83 (m, 2H), 0.81-0.73 (m, 2H)

### Example 353

### (3R,4S)-3-cyclopropyl-1-(6-(1-(difluoromethyl)-1H-pyrazol-4-yl)thiazolo[5,4-c]pyridin-4-yl)-4-methyl-2-oxopyrrolidine-3-carbonitrile

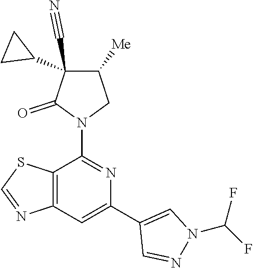 [SMILES: C[C@@H]1CN(c2nc(-c3cnn(C(F)F)c3)cc3ncsc23)C(=O)[C@]1(C#N)C1CC1]

(3R,4S)-3-Cyclopropyl-1-(6-(1-(difluoromethyl)-1H-pyrazol-4-yl)thiazolo[5,4-c]pyridin-4-yl)-4-methyl-2-oxopyrrolidine-3-carbonitrile was obtained as a yellow solid, 10.4 mg, 45.7% yield, from (3R,4S)-1-(6-(1H-pyrazol-4-yl)thiazolo[5,4-c]pyridin-4-yl)-3-cyclopropyl-4-methyl-2-oxopyrrolidine-3-carbonitrile (Preparation 248) and sodium 2-chloro-2,2-difluoroacetate, following a similar procedure to that described in Example 352, except the crude product was purified by Prep-HPLC-3 (Gradient: 35-55%). LCMS m/z=415.1 [M+H]⁺; ¹H NMR (400 MHz, CDCl₃) δ: =9.23 (d, J=2.0 Hz, 1H), 8.37 (s, 1H), 8.20 (s, 1H), 8.11 (d, J=1.6 Hz, 1H), 7.45-7.18 (m, 1H), 4.39-4.34 (m, 1H), 4.05-3.98 (m, 1H), 1.49-1.47 (m, 3H), 1.25-1.20 (m, 1H), 0.90-0.67 (m, 5H)

### Example 354

### (3R,4S)-3-cyclopropyl-1-(6-(1-(2,2-difluoroethyl)-1H-pyrazol-4-yl)thiazolo[5,4-c]pyridin-4-yl)-4-methyl-2-oxopyrrolidine-3-carbonitrile

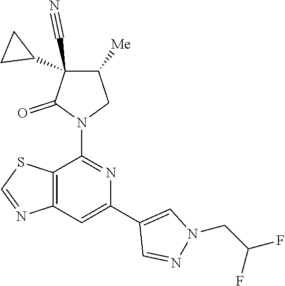 [SMILES: C[C@@H]1CN(c2nc(-c3cnn(CC(F)F)c3)cc3ncsc23)C(=O)[C@]1(C#N)C1CC1]

To a solution of (3R,4S)-1-(6-(1H-pyrazol-4-yl)thiazolo[5,4-c]pyridin-4-yl)-3-cyclopropyl-4-methyl-2-oxopyrrolidine-3-carbonitrile (Preparation 248, 30 mg, 0.0823 mmol) in DMF (3 mL) was added Cs₂CO₃ (60 mg, 0.184 mmol) and 1,1-difluoro-2-iodoethane (45 mg, 0.234 mmol) and the reaction was stirred at 40° C. for 2 h. The reaction was concentrated in vacuo and the residue was purified by Prep-HPLC-20 (gradient: 50 to 60%) to give (3R,4S)-3-cyclopropyl-1-(6-(1-(2,2-difluoroethyl)-1H-pyrazol-4-yl)thiazolo[5,4-c]pyridin-4-yl)-4-methyl-2-oxopyrrolidine-3-carbonitrile (12.9 mg, 36.6% yield) as a yellow solid. LCMS m/z=429.1 [M+H]⁺; ¹H NMR (400 MHz, CDCl₃) δ=9.22 (s, 1H), 8.13 (s, 1H), 8.07-8.05 (m, 2H), 6.30-6.08 (m, 1H), 4.61-4.53 (m, 2H), 4.40-4.35 (m, 1H), 4.05-4.00 (m, 1H), 3.18-3.13 (m, 1H), 1.49 (d, J=6.8 Hz, 3H), 1.26-1.22 (m, 1H), 0.87-0.83 (m, 3H), 0.79-0.76 (m, 1H).

### Example 355

### (3R,4S)-3-cyclopropyl-1-(6-(1-((1r,3R)-3-methoxycyclobutyl)-1H-pyrazol-4-yl)thiazolo[5,4-c]pyridin-4-yl)-4-methyl-2-oxopyrrolidine-3-carbonitrile

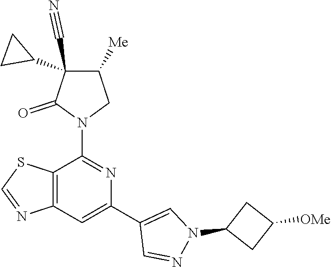 [SMILES: CO[C@H]1C[C@H](n2cc(-c3cc4ncsc4c(N4C[C@@H](C)[C@@](C#N)(C5CC5)C4=O)n3)cn2)C1]

To a solution of (3R,4S)-1-(6-(1H-pyrazol-4-yl)thiazolo[5,4-c]pyridin-4-yl)-3-cyclopropyl-4-methyl-2-oxopyrrolidine-3-carbonitrile (Preparation 248, 30 mg, 0.82 mmol) in DMF (1 mL) was added Cs₂CO₃ (80.47 mg, 247 mmol) and (1s,3s)-3-methoxycyclobutyl methanesulfonate (16.32 mg, 0.091 mmol) and the reaction was stirred at 90° C. for 1 h. The mixture was concentrated under vacuum to give the crude, which was purified by Prep-HPLC-20 (gradient: 45 to 65%) to give (3R,4S)-3-cyclopropyl-1-(6-(1-((1r,3R)-3-methoxycyclobutyl)-1H-pyrazol-4-yl)thiazolo[5,4-c]pyridin-4-yl)-4-methyl-2-oxopyrrolidine-3-carbonitrile (7.3 mg, 19.8% yield) as a yellow solid. The stereochemistry was determined by 2D nmr. LCMS m/z=449.1 [M+H]⁺; ¹H NMR (400 MHz, CDCl₃) δ=9.18 (d, J=1.2 Hz, 1H), 8.08 (s, 1H), 8.04 (d, J=1.2 Hz, 1H), 7.96 (s, 1H), 5.00-4.97 (m, 1H), 4.38-4.34 (m, 1H), 4.28 (d, J=3.2 Hz, 1H), 4.00 (t, J=10.0 Hz, 1H), 3.34-3.33 (m, 3H), 3.16-3.11 (m, 1H), 2.87-2.82 (m, 2H), 2.64-2.60 (m, 2H), 1.48-1.46 (m, 3H), 1.24-1.20 (m, 1H), 0.86-0.77 (m, 4H)

### Example 356

### (3R,4S)-1-(6-(1-(3-cyanocyclobutyl)-1H-pyrazol-4-yl)thiazolo[5,4-c]pyridin-4-yl)-3-cyclopropyl-4-methyl-2-oxopyrrolidine-3-carbonitrile

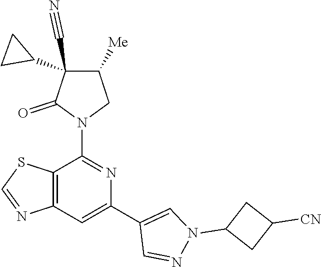 [SMILES: C[C@@H]1CN(c2nc(-c3cnn(C4CC(C#N)C4)c3)cc3ncsc23)C(=O)[C@]1(C#N)C1CC1]

(3R,4S)-1-(6-(1-(3-Cyanocyclobutyl)-1H-pyrazol-4-yl)thiazolo[5,4-c]pyridin-4-yl)-3-cyclopropyl-4-methyl-2-oxopyrrolidine-3-carbonitrile was obtained as a gray solid, 8.8 mg, 24.1% yield, from (3R,4S)-1-(6-(1H-pyrazol-4-yl)thiazolo[5,4-c]pyridin-4-yl)-3-cyclopropyl-4-methyl-2-oxopyrrolidine-3-carbonitrile (Preparation 248) and 3-cyanocyclobutyl methanesulfonate, following a similar procedure to that described in Example 355, except the crude was purified by HPLC-3 (Gradient: 35 to 64%). LCMS m/z=444.1 [M+H]⁺;

¹H NMR: (500 MHz, MeOD-d₄) δ=9.50 (s, 0.25H), 9.45 (s, 0.75H), 8.83 (s, 0.25H), 8.54 (s, 0.25H), 8.39 (s, 0.75H), 8.29 (s, 0.25H), 8.21 (s, 0.75H), 8.15-8.13 (m, 0.75H), 5.32-5.28 (m, 1H), 4.50-4.44 (m, 1H), 4.05-3.99 (m, 1H), 3.51-3.48 (m, 1H), 3.10-2.90 (m, 5H), 1.47-1.42 (m, 4H), 0.82-0.76 (m, 1H), 0.76-0.71 (m, 3H).

### Example 357

### (3R,4S)-3-cyclopropyl-1-(6-(1-cyclopropyl-1H-pyrazol-4-yl)thiazolo[5,4-c]pyridin-4-yl)-4-methyl-2-oxopyrrolidine-3-carbonitrile

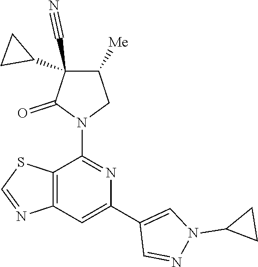 [SMILES: C[C@@H]1CN(c2nc(-c3cnn(C4CC4)c3)cc3ncsc23)C(=O)[C@]1(C#N)C1CC1]

To a mixture of (3R,4S)-1-(6-(1H-pyrazol-4-yl)thiazolo[5,4-c]pyridin-4-yl)-3-cyclopropyl-4-methyl-2-oxopyrrolidine-3-carbonitrile (Preparation 248, 50 mg, 0.137 mmol), cyclopropyl boronic acid (17.68 mg, 0.206 mmol) and K₃PO₄ (87.37 mg, 0.412 mmol) in DCE (2 mL) and water (0.2 mL) was added 1,10-phenanthroline (29.67 mg, 0.165 mmol) and Cu(OAc)₂ (29.90 mg, 0.165 mmol) and the reaction mixture was stirred at 90° C. for 2 h. The reaction mixture was concentrated in vacuo and the crude product was purified by prep-HPLC-3 (32 to 60% gradient) to give (3R,4S)-3-cyclopropyl-1-(6-(1-cyclopropyl-1H-pyrazol-4-yl)thiazolo[5,4-c]pyridin-4-yl)-4-methyl-2-oxopyrrolidine-3-carbonitrile (28.3 mg, 51.0% yield) as a white solid. LCMS m/z=405.1 [M+H]⁺; 1H NMR (500 MHz, DMSO-d₆) δ=9.62-9.61 (m, 1H), 8.52-8.49 (m, 1H), 8.29-8.28 (m, 1H), 8.25-8.14 (m, 1H), 4.44-4.40 (m, 1H), 3.97-3.79 (m, 2H), 3.19-3.16 (m, 1H), 1.50-1.46 (m, 1H), 1.33 (d, J=7.0 Hz, 3H), 1.13-1.01 (m, 4H), 0.77-0.74 (m, 1H), 0.69-0.66 (m, 1H), 0.60-0.58 (m, 2H).

### Example 358

### (3R,4S)-3-cyclopropyl-4-methyl-1-(6-(1-(1-methylazetidin-3-yl)-1H-pyrazol-4-yl)thiazolo[5,4-c]pyridin-4-yl)-2-oxopyrrolidine-3-carbonitrile

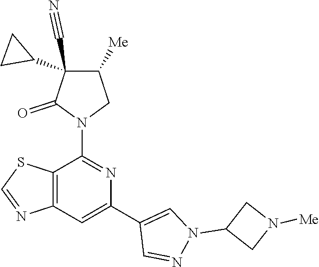 [SMILES: C[C@@H]1CN(c2nc(-c3cnn(C4CN(C)C4)c3)cc3ncsc23)C(=O)[C@]1(C#N)C1CC1]

Part A: To a solution of tert-butyl 3-(4-(4-((3R,4S)-3-cyano-3-cyclopropyl-4-methyl-2-oxopyrrolidin-1-yl)thiazolo[5,4-c]pyridin-6-yl)-1H-pyrazol-1-yl)azetidine-1-carboxylate (Preparation 271, 19 mg, 0.0366 mmol) in DCM (1 mL) and 1,1,1,3,3,3-hexafluoropropan-2-ol (5 mL) was added TFA (297.80 mg, 2.61 mmol) and the mixture was stirred at 20° C. for 30 min. The mixture was concentrated under vacuum to give (3R,4S)-1-(6-(1-(azetidin-3-yl)-1H-pyrazol-4-yl)thiazolo[5,4-c]pyridin-4-yl)-3-cyclopropyl-4-methyl-2-oxopyrrolidine-3-carbonitrile trifluoroactate (11.65 mg, crude) as yellow oil which was used for the next step without further purification. LCMS m/z=420.2 [M+H]⁺

Part B: To a solution of (3R,4S)-1-(6-(1-(azetidin-3-yl)-1H-pyrazol-4-yl)thiazolo[5,4-c]pyridin-4-yl)-3-cyclopropyl-4-methyl-2-oxopyrrolidine-3-carbonitrile trifluoroactate (11.65 mg, 0.0278 mmol) in MeOH (2 mL) was added (CH₂O)₃ (33.31 mg, 0.0278 mmol) and the reaction was stirred at 20° C. for 30 mins. NaBH₃CN (5.24 mg, 0.083 mmol) was added and the reaction stirred for 3 h. The mixture was concentrated under vacuum to give the crude, which was purified by Prep-HPLC-20 (gradient: 11 to 31%) to give (3R,4S)-3-cyclopropyl-4-methyl-1-(6-(1-(1-methylazetidin-3-yl)-1H-pyrazol-4-yl)thiazolo[5,4-c]pyridin-4-yl)-2-oxopyrrolidine-3-carbonitrile (7 mg, 58.14% yield) as a yellow solid. LCMS m/z=434.1 [M+H]⁺; ¹H NMR (500 MHz, CDCl₃) δ=9.22-9.17 (m, 1H), 8.15-8.12 (m, 1H), 8.10-8.07 (m, 1H), 8.05-8.03 (m, 1H), 5.34-5.28 (m, 1H), 4.49-4.46 (m, 2H), 4.36-4.32 (m, 1H), 4.10 (t, J=8.0 Hz, 2H), 4.00 (t, J=10.5 Hz, 1H), 3.15-3.11 (m, 1H), 2.86-2.85 (m, 3H), 1.49-1.46 (m, 3H), 1.26-1.21 (m, 1H), 0.85-0.73 (m, 4H).

### Biological Assays

### Biochemical Assay

Compounds of the disclosure were assessed for their ability to inhibit TYK2, JAK1, JAK2, and JAK3 activity. The inhibitory properties of the compounds of the disclosure described herein can be evidenced by testing in any one of the following protocols.

### JH1 Biochemical Assay for TYK2, JAK1, JAK2, and JAK3

The kinase activity of recombinantly generated catalytic kinase (also known as JH1) domain of human JAK1, JAK2, JAK3 and TYK2 were evaluated in a plate-based assay using the ADP-Glo™ Kinase Assay platform. Specifically, 4 nM of recombinant JAK1 kinase domain is used to phosphorylate 50 μM of a JAK3-342 (sequence ALVDGYFRLTT) peptide in the presence of 35 μM ATP. Catalytic activities of recombinant JAK2, JAK3 and TYK2 kinase domain (0.2, 0.3 and 2 nM, respectively) are evaluated by the phosphorylation status of the JAK3-974 (50 μM; sequence LPLDKDYYVVR) peptide with the addition of ATP (15, 4 and 10 μM, respectively). The reactions proceed for 100 minutes and the catalytic activity is quantified by first depleting the unused ATP, converting the hydrolyzed ADP into ATP to generate luminescence in a luciferase reaction; which is the basis of the ADP-Glo platform. Compounds are tested at either 10 μM or 1 μM top concentration, 11 points of 3-fold dilution. The data is normalized and the percent activity versus log concentration of compound is fitted with a 4-parameter logistic model to generate a curve and an IC₅₀ value.

pSTAT4 Cell Assay

The inhibitory potency of compounds of the disclosure against the Tyk2 kinase activity on STAT4 was evaluated using an MSD-platform plate-based assay format. NK92 cells natively expressing STAT4 and Tyk2 were serum-starved to reduce background phosphorylation levels, then cells were treated compounds for 1 hr with a 10-point four-fold dilution series starting at 10 μM. Cells were then stimulated with 30 ng/mL IL2 for 15 minutes. Cells were lysed and pSTAT5 levels were quantitated using an MSD plate-based assay with anti-STAT4 antibodies. The data were normalized and the percent activity versus log concentration of compound were fitted with a 4-parameter logistic model to generate to generate IC₅₀ curves.

pSTAT5 Cell Assay

The inhibitory potency of compounds of the disclosure against the JAK2 kinase activity on STAT5 was evaluated using an MSD-platform plate-based assay format. TF1 cells natively expressing STAT5 and JAK2 were serum-starved to reduce background phosphorylation levels, then cells were treated with compounds of disclosure for 1 hour with a 10-point four-fold dilution series starting at 10 μM. Cells were then stimulated with 30 ng/mL IL-3 for 15 minutes. Cells were then lysed and pSTAT5 levels were quantitated using an MSD plate-based assay with anti-STAT5 antibodies. The data were normalized and the percent activity versus log concentration of compound was fitted with a 4-parameter logistic model to generate a curve and an IC₅₀ value.

pSTAT3 Cell Assay

The inhibitory potency of compounds of the disclosure against the JAK1 kinase activity on STAT3 was evaluated using an MSD-platform plate-based assay format. TF1 cells natively expressing STAT3 and JAK1 were serum-starved to reduce background phosphorylation levels, then cells were treated with compounds of the disclosure for 1 hour with a 10-point four-fold dilution series starting at 10 μM. Cells were then stimulated with 30 ng/mL interleukin 6 (IL-6) for 15 minutes. Cells were lysed and pSTAT3 levels were quantitated using an MSD plate-based assay with anti-STAT3 antibodies. The data were normalized and the percent activity versus log concentration of compound was fitted with a 4-parameter logistic model to generate IC₅₀ curves.

| | TYK2 | JAK1 | JAK2 | JAK3 | pSTAT4 | pSTAT5 | pSTAT3 |
|---|---|---|---|---|---|---|---|
| Example | IC₅₀ | IC₅₀ | IC₅₀ | IC₅₀ | IC₅₀ | IC₅₀ | IC₅₀ |
| No. | (nM) | (nM) | (nM) | (nM) | (nM) | (nM) | (nM) |
| 1 | ***** | + | + | + | **** | ++ | + |
| 2 | ** | ++ | ++ | +++ | NT | NT | NT |
| 3 | ***** | + | + | + | *** | +++ | ++ |
| 4 | ***** | + | + | + | *** | ++ | ++ |
| 5 | * | +++ | ++ | +++ | * | +++++ | ++++ |
| 6 | ** | +++ | +++ | +++ | * | +++++ | +++++ |
| 7 | * | +++++ | ++++ | ++++ | NT | NT | NT |
| 8 | **** | ++ | ++ | ++ | * | +++ | +++ |
| 9 | **** | ++ | ++ | ++ | * | ++++ | +++ |
| 10 | **** | ++ | ++ | ++ | * | +++++ | +++ |
| 11 | *** | +++ | +++ | ++ | * | +++++ | +++++ |
| 12 | **** | ++ | ++ | ++ | * | +++++ | +++ |
| 13 | * | ++++ | +++++ | +++++ | NT | NT | NT |
| 14 | * | +++++ | ++++ | +++++ | NT | NT | NT |
| 15 | * | +++ | +++ | +++ | NT | NT | NT |
| 16 | * | ++++ | ++ | +++++ | NT | NT | NT |
| 17 | * | +++ | ++ | ++++ | NT | NT | NT |
| 18 | * | +++ | ++ | +++ | NT | NT | NT |
| 19 | * | +++++ | +++++ | +++++ | NT | NT | NT |
| 20 | * | +++++ | ++++ | +++++ | NT | NT | NT |
| 21 | * | +++++ | +++++ | +++++ | NT | NT | NT |
| 22 | ** | +++ | +++ | +++ | * | +++++ | +++++ |
| 23 | **** | ++ | ++ | ++ | ** | ++++ | +++ |
| 24 | * | ++++ | ++++ | +++++ | NT | NT | NT |
| 25 | * | +++++ | +++++ | +++++ | NT | NT | NT |
| 26 | * | +++++ | +++++ | +++++ | NT | NT | NT |
| 27 | * | +++ | +++ | +++ | NT | NT | NT |
| 28 | ** | +++ | ++++ | +++++ | * | +++++ | +++++ |
| 29 | * | +++++ | +++++ | +++++ | NT | NT | NT |
| 30 | * | +++++ | +++++ | +++++ | NT | NT | NT |
| 31 | * | +++++ | +++++ | +++++ | NT | NT | NT |
| 32 | * | +++++ | +++++ | +++++ | NT | NT | NT |
| 33 | * | ++++ | +++ | ++++ | NT | NT | NT |
| 34 | ***** | ++ | ++ | ++ | *** | ++++ | +++ |
| 35 | ***** | ++ | ++ | ++ | *** | ++++ | +++ |
| 36 | * | +++++ | ++++ | +++++ | NT | NT | NT |
| 37 | * | +++++ | +++ | +++ | NT | NT | NT |
| 38 | ** | +++ | +++ | +++ | NT | NT | NT |
| 39 | * | +++++ | +++++ | +++++ | NT | NT | NT |
| 40 | ** | +++ | ++ | ++ | * | +++++ | +++++ |
| 41 | * | +++++ | ++++ | ++++ | NT | NT | NT |
| 42 | ** | +++ | +++ | +++ | NT | NT | NT |
| 43 | * | +++++ | +++++ | +++++ | NT | NT | NT |
| 44 | * | +++++ | +++++ | +++++ | NT | NT | NT |
| 45 | * | +++++ | +++++ | +++++ | NT | NT | NT |
| 46 | * | ++++ | +++ | ++ | NT | NT | NT |
| 47 | * | +++++ | ++++ | +++++ | NT | NT | NT |
| 48 | **** | ++ | ++ | ++ | * | ++++ | +++ |
| 49 | *** | ++ | +++ | +++ | NT | NT | NT |
| 50 | ** | +++ | +++ | +++ | NT | NT | NT |
| 51 | **** | + | ++ | ++ | ** | +++++ | +++ |
| 52 | **** | ++ | ++ | ++ | * | +++++ | ++++ |
| 53 | **** | ++ | ++ | ++ | * | +++++ | ++++ |
| 54 | **** | ++ | +++ | ++ | * | +++++ | ++++ |
| 55 | ***** | + | ++ | ++ | **** | +++ | ++ |
| 56 | **** | ++ | ++ | ++ | * | +++++ | ++++ |
| 57 | **** | ++ | ++ | ++ | * | +++++ | +++ |
| 58 | **** | ++ | ++ | ++ | ** | +++++ | +++ |
| 59 | ***** | + | ++ | ++ | ** | ++++ | +++ |
| 60 | **** | ++ | ++ | ++ | * | +++++ | +++ |
| 61 | ***** | + | ++ | ++ | *** | ++++ | ++ |
| 62 | ***** | ++ | ++ | ++ | ** | +++ | +++ |
| 63 | **** | ++ | ++ | ++ | * | +++++ | +++++ |
| 64 | **** | ++ | ++ | ++ | ** | ++++ | +++ |
| 65 | **** | ++ | ++ | ++ | ** | +++++ | +++ |
| 66 | **** | ++ | ++ | ++ | * | +++++ | ++++ |
| 67 | **** | ++ | ++ | ++ | * | ++++ | +++ |
| 68 | **** | + | ++ | ++ | ** | ++++ | ++ |
| 69 | **** | ++ | +++ | ++ | * | +++++ | ++++ |
| 70 | ** | +++ | +++ | +++ | NT | NT | NT |
| 71 | **** | ++ | ++ | ++ | * | +++++ | +++ |
| 72 | **** | ++ | ++ | ++ | * | ++++ | +++ |
| 73 | **** | ++ | ++ | ++ | * | +++++ | +++ |
| 74 | **** | ++ | +++ | ++ | * | +++++ | ++++ |
| 75 | ***** | + | ++ | ++ | ** | +++++ | +++ |
| 76 | ** | ++ | +++ | +++ | NT | NT | NT |
| 77 | ***** | + | ++ | ++ | ** | +++ | ++ |
| 78 | * | +++++ | +++++ | +++++ | NT | NT | NT |
| 79 | **** | ++ | ++ | ++ | * | +++++ | +++++ |
| 80 | ** | +++ | +++ | +++ | NT | NT | NT |
| 81 | **** | ++ | ++ | ++ | * | +++++ | ++++ |
| 82 | *** | +++++ | +++++ | +++++ | NT | NT | NT |
| 83 | ***** | + | ++ | ++ | ** | ++++ | +++ |
| 84 | **** | ++ | +++ | ++ | * | +++++ | ++++ |
| 85 | *** | +++ | +++ | +++ | NT | NT | NT |
| 86 | **** | ++ | +++ | ++ | * | +++++ | ++++ |
| 87 | *** | ++++ | +++++ | +++++ | NT | NT | NT |
| 88 | ***** | + | ++ | ++ | *** | +++ | ++ |
| 89 | *** | +++ | +++ | +++ | NT | NT | NT |
| 90 | **** | ++ | ++ | ++ | * | +++++ | ++++ |
| 91 | *** | ++ | +++ | +++ | NT | NT | NT |
| 92 | ** | +++ | ++++ | +++ | NT | NT | NT |
| 93 | **** | ++ | ++ | ++ | * | +++++ | ++++ |
| 94 | *** | +++ | +++++ | +++++ | NT | NT | NT |
| 95 | **** | ++ | ++ | ++ | NT | NT | NT |
| 96 | **** | +++ | +++ | +++ | * | +++++ | +++++ |
| 97 | ***** | + | + | + | *** | +++ | +++ |
| 98 | ** | +++ | +++ | ++++ | NT | NT | NT |
| 99 | *** | ++ | +++ | ++ | NT | NT | NT |
| 100 | ** | +++ | +++ | +++ | NT | NT | NT |
| 101 | **** | ++ | ++ | ++ | * | +++++ | +++ |
| 102 | **** | ++ | +++ | ++ | * | +++++ | ++++ |
| 103 | **** | +++++ | +++++ | +++++ | * | +++++ | +++++ |
| 104 | ***** | ++ | ++ | ++ | * | ++++ | +++ |
| 105 | * | +++++ | +++ | ++++ | NT | NT | NT |
| 106 | ***** | ++ | ++ | ++ | * | +++++ | +++++ |
| 107 | *** | +++ | +++ | +++ | NT | NT | NT |
| 108 | ** | +++ | +++++ | +++ | NT | NT | NT |
| 109 | ** | +++ | ++++ | +++ | NT | NT | NT |
| 110 | **** | ++ | ++ | ++ | * | +++++ | +++++ |
| 111 | ***** | ++ | ++ | ++ | * | +++++ | +++++ |
| 112 | **** | ++ | ++ | ++ | * | +++++ | +++++ |
| 113 | *** | ++ | +++ | ++ | NT | NT | NT |
| 114 | *** | +++++ | +++++ | +++ | NT | NT | NT |
| 115 | * | +++++ | +++++ | ++++ | NT | NT | NT |
| 116 | *** | ++ | +++ | ++ | NT | NT | NT |
| 117 | *** | +++ | +++ | +++ | NT | NT | NT |
| 118 | **** | ++ | ++ | ++ | * | +++++ | ++++ |
| 119 | *** | +++ | +++ | ++ | NT | NT | NT |
| 120 | * | ++++ | +++++ | +++ | NT | NT | NT |
| 121 | * | +++ | ++++ | +++ | NT | NT | NT |
| 122 | * | ++++ | +++++ | +++++ | NT | NT | NT |
| 123 | * | +++++ | +++++ | +++++ | NT | NT | NT |
| 124 | **** | ++ | ++ | ++ | * | ++++ | ++++ |
| 125 | *** | ++ | ++ | ++ | NT | NT | NT |
| 126 | *** | ++ | ++ | ++ | NT | NT | NT |
| 127 | **** | ++ | ++ | ++ | * | +++++ | ++++ |
| 128 | **** | ++ | +++ | ++ | * | +++++ | +++++ |
| 129 | * | +++++ | +++++ | +++++ | NT | NT | NT |
| 130 | **** | ++ | ++ | ++ | NT | NT | NT |
| 131 | ***** | + | ++ | ++ | ** | +++ | ++ |
| 132 | ** | +++ | +++ | +++ | NT | NT | NT |
| 133 | *** | +++ | +++ | +++ | NT | NT | NT |
| 134 | **** | ++ | ++ | ++ | * | ++++ | +++ |
| 135 | **** | ++ | ++ | ++ | * | +++++ | +++ |
| 136 | ** | +++ | ++++ | +++ | NT | NT | NT |
| 137 | *** | +++ | ++ | ++ | NT | NT | NT |
| 138 | *** | ++ | +++++ | +++ | NT | NT | NT |
| 139 | * | +++ | +++++ | +++ | NT | NT | NT |
| 140 | ** | +++ | +++++ | ++++ | NT | NT | NT |
| 141 | *** | +++ | +++ | +++ | NT | NT | NT |
| 142 | *** | +++ | +++ | +++ | NT | NT | NT |
| 143 | *** | ++ | +++ | ++ | NT | NT | NT |
| 144 | *** | +++ | +++ | +++ | NT | NT | NT |
| 145 | **** | ++ | ++ | ++ | * | +++++ | ++++ |
| 146 | * | +++ | ++++ | ++++ | NT | NT | NT |
| 147 | **** | ++ | ++ | ++ | NT | NT | NT |
| 148 | *** | ++ | ++ | ++ | NT | NT | NT |
| 149 | *** | +++ | ++++ | +++ | NT | NT | NT |
| 150 | **** | ++ | ++ | ++ | NT | NT | NT |
| 151 | ** | +++ | +++ | +++ | NT | NT | NT |
| 152 | * | +++ | +++ | +++ | NT | NT | NT |
| 153 | *** | ++ | +++ | +++ | * | +++++ | +++++ |
| 154 | ***** | + | ++ | ++ | **** | +++ | +++ |
| 155 | ***** | + | ++ | ++ | ** | +++ | +++ |
| 156 | ***** | ++ | ++ | + | ** | +++ | +++ |
| 157 | ***** | + | + | + | ** | +++ | ++ |
| 158 | ***** | ++ | ++ | ++ | * | +++++ | +++ |
| 159 | ***** | ++ | ++ | ++ | ** | ++++ | +++ |
| 160 | ***** | ++ | ++ | ++ | ** | +++ | +++ |
| 161 | ***** | ++ | ++ | ++ | *** | +++ | +++ |
| 162 | ***** | + | + | + | *** | +++ | +++ |
| 163 | ***** | ++ | ++ | ++ | * | ++++ | +++ |
| 164 | ***** | + | ++ | + | *** | ++++ | +++ |
| 165 | ***** | ++ | ++ | ++ | *** | ++++ | +++ |
| 166 | **** | ++ | ++ | ++ | * | +++++ | +++++ |
| 167 | ***** | + | + | + | *** | +++ | ++ |
| 168 | ***** | + | + | + | ** | +++ | +++ |
| 169 | ***** | + | + | + | * | +++ | +++ |
| 170 | ***** | + | + | + | * | ++++ | ++++ |
| 171 | **** | ++ | ++ | ++ | * | +++++ | ++++ |
| 172 | ***** | ++ | ++ | ++ | * | ++++ | +++ |
| 173 | **** | ++ | ++ | ++ | NT | NT | NT |
| 174 | **** | ++ | ++ | ++ | ** | ++++ | +++ |
| 175 | * | ++++ | +++++ | +++++ | NT | NT | NT |
| 176 | **** | ++ | +++ | ++ | * | +++++ | ++++ |
| 177 | *** | ++ | +++ | +++ | NT | NT | NT |
| 178 | * | ++++ | +++++ | ++++ | NT | NT | NT |
| 179 | * | +++++ | +++++ | +++++ | NT | NT | NT |
| 180 | * | +++++ | ++++ | +++++ | * | ++++ | +++ |
| 181 | **** | ++ | ++ | ++ | * | ++++ | +++ |
| 182 | *** | ++ | ++ | ++ | * | +++++ | ++++ |
| 183 | * | +++++ | +++++ | +++++ | NT | NT | NT |
| 184 | ***** | + | + | + | *** | ++ | ++ |
| 185 | ***** | + | + | + | *** | ++ | ++ |
| 186 | ** | +++ | +++ | ++ | * | +++++ | +++++ |
| 187 | ***** | + | ++ | + | ** | +++ | +++ |
| 188 | ***** | + | + | + | *** | +++ | ++ |
| 189 | ***** | + | ++ | + | ** | +++ | +++ |
| 190 | ***** | + | + | + | *** | +++ | ++ |
| 191 | ***** | + | + | + | ** | +++ | +++ |
| 192 | **** | + | ++ | + | * | +++++ | +++ |
| 193 | **** | ++ | +++++ | +++++ | * | +++++ | +++++ |
| 194 | ***** | + | ++ | + | * | +++ | +++ |
| 195 | **** | + | ++ | + | * | +++++ | +++ |
| 196 | *** | ++ | +++ | ++ | NT | NT | NT |
| 197 | **** | + | ++ | + | * | +++++ | +++ |
| 198 | ***** | + | + | + | ** | ++ | ++ |
| 199 | **** | + | ++ | ++ | * | ++++ | +++ |
| 200 | *** | ++ | +++ | ++ | NT | NT | NT |
| 201 | ***** | + | + | + | *** | ++ | ++ |
| 202 | ***** | + | + | + | ** | ++ | ++ |
| 203 | ***** | + | + | + | *** | + | ++ |
| 204 | ***** | + | + | + | ** | +++ | +++ |
| 205 | ***** | + | + | + | * | ++++ | +++ |
| 206 | **** | ++ | ++ | ++ | * | +++++ | +++++ |
| 207 | ***** | + | ++ | + | ** | +++ | ++ |
| 208 | ***** | + | + | + | ** | +++ | +++ |
| 209 | **** | + | ++ | + | * | ++++ | +++ |
| 210 | ***** | + | ++ | + | * | ++++ | +++ |
| 211 | ***** | + | ++ | + | ** | +++ | +++ |
| 212 | ** | +++ | +++++ | +++ | NT | NT | NT |
| 213 | **** | + | +++ | ++ | * | NT | NT |
| 214 | **** | ++ | ++ | ++ | * | ++++ | +++ |
| 215 | ***** | + | + | + | ** | ++ | ++ |
| 216 | * | +++++ | +++++ | +++++ | NT | NT | NT |
| 217 | ***** | + | + | + | * | +++ | ++ |
| 218 | ***** | + | + | + | ** | ++ | ++ |
| 219 | ***** | + | + | + | ** | ++ | ++ |
| 220 | ***** | ++ | ++ | + | * | +++ | +++ |
| 221 | **** | ++ | +++ | ++ | * | +++++ | +++ |
| 222 | ** | +++ | +++ | ++ | NT | NT | NT |
| 223 | ** | +++ | +++ | +++ | NT | NT | NT |
| 224 | *** | ++ | +++ | +++ | NT | NT | NT |
| 225 | ***** | + | ++ | + | * | +++ | +++ |
| 226 | * | ++++ | +++++ | +++++ | NT | NT | NT |
| 227 | ***** | + | ++ | + | * | +++ | ++ |
| 228 | ***** | + | ++ | + | ** | +++ | ++ |
| 229 | * | +++ | +++++ | +++ | NT | NT | NT |
| 230 | ***** | + | ++ | + | **** | +++ | ++ |
| 231 | * | +++++ | +++++ | +++++ | NT | NT | NT |
| 232 | **** | + | ++ | ++ | * | +++++ | +++ |
| 233 | ***** | + | ++ | + | * | +++ | +++ |
| 234 | **** | ++ | ++ | ++ | * | +++++ | +++++ |
| 235 | ***** | + | ++ | ++ | * | +++++ | +++++ |
| 236 | **** | ++ | ++ | ++ | * | ++++ | +++ |
| 237 | **** | + | ++ | ++ | ** | ++++ | ++ |
| 238 | * | +++++ | +++++ | +++++ | NT | NT | NT |
| 239 | ***** | + | ++ | + | ** | +++ | ++ |
| 240 | * | +++++ | +++++ | +++++ | NT | NT | NT |
| 241 | ** | ++ | +++ | +++ | NT | NT | NT |
| 242 | * | +++++ | +++++ | +++++ | NT | NT | NT |
| 243 | * | +++ | +++++ | +++ | NT | NT | NT |
| 244 | **** | ++ | ++ | ++ | * | +++++ | ++++ |
| 245 | ***** | + | ++ | + | ** | +++ | ++ |
| 246 | **** | ++ | ++ | ++ | NT | NT | NT |
| 247 | * | +++++ | +++++ | +++++ | NT | NT | NT |
| 248 | *** | ++ | +++ | +++ | NT | NT | NT |
| 249 | * | +++++ | +++++ | ++++ | NT | NT | NT |
| 250 | *** | ++ | +++ | +++ | NT | NT | NT |
| 251 | ***** | + | + | + | *** | ++ | ++ |
| 252 | **** | ++ | ++ | ++ | * | +++++ | +++ |
| 253 | *** | +++ | +++++ | ++++ | NT | NT | NT |
| 254 | *** | ++ | ++ | ++ | NT | NT | NT |
| 255 | *** | + | +++++ | ++ | NT | NT | NT |
| 256 | * | +++++ | +++ | +++ | NT | NT | NT |
| 257 | **** | + | ++ | ++ | * | ++++ | +++ |
| 258 | **** | + | ++ | + | * | +++++ | +++ |
| 259 | ***** | + | + | + | **** | ++ | + |
| 260 | ** | ++ | +++ | +++ | NT | NT | NT |
| 261 | ***** | ++ | ++ | + | * | +++ | +++ |
| 262 | *** | +++++ | +++++ | +++++ | NT | NT | NT |
| 263 | ***** | + | + | + | *** | ++ | + |
| 264 | **** | ++ | +++ | ++ | ** | +++++ | +++++ |
| 265 | ** | +++ | +++ | +++ | NT | NT | NT |
| 266 | ***** | + | + | + | ** | NT | NT |
| 267 | ***** | + | ++ | + | ** | ++ | ++ |
| 268 | **** | ++ | ++ | ++ | ** | +++++ | +++ |
| 269 | * | +++ | ++++ | +++ | NT | NT | NT |
| 270 | *** | +++ | +++ | +++ | NT | NT | NT |
| 271 | **** | ++ | +++ | ++ | * | +++++ | +++++ |
| 272 | * | +++++ | +++++ | +++++ | NT | NT | NT |
| 273 | * | ++++ | +++++ | ++++ | NT | NT | NT |
| 274 | * | +++++ | ++ | +++++ | NT | NT | NT |
| 275 | **** | ++ | +++ | ++ | * | +++++ | ++++ |
| 276 | **** | ++ | +++ | ++ | * | +++++ | ++++ |
| 277 | ***** | ++ | ++ | ++ | * | ++++ | +++ |
| 278 | ***** | ++ | ++ | ++ | * | ++++ | ++++ |
| 279 | * | +++++ | +++++ | +++++ | NT | NT | NT |
| 280 | ***** | ++ | ++ | ++ | * | +++++ | +++ |
| 281 | **** | ++ | +++ | ++ | * | +++++ | +++ |
| 282 | *** | +++ | +++ | +++ | NT | NT | NT |
| 283 | * | +++++ | +++++ | +++++ | NT | NT | NT |
| 284 | ***** | + | + | + | **** | + | + |
| 285 | * | +++++ | +++++ | +++++ | NT | NT | NT |
| 286 | * | +++++ | +++ | +++++ | NT | NT | NT |
| 287 | * | +++++ | +++++ | +++++ | NT | NT | NT |
| 288 | **** | ++ | + | ++ | ** | +++ | +++ |
| 289 | ***** | + | + | + | **** | + | + |
| 290 | ***** | + | + | + | *** | ++ | ++ |
| 291 | ***** | + | + | + | ** | +++ | +++ |
| 292 | ***** | + | + | + | *** | ++ | ++ |
| 293 | ***** | + | + | + | *** | ++ | ++ |
| 294 | ***** | + | + | + | *** | ++ | ++ |
| 295 | ***** | + | + | + | *** | ++ | ++ |
| 296 | ***** | + | ++ | + | *** | +++ | ++ |
| 297 | ***** | + | + | + | * | +++ | +++ |
| 298 | ***** | + | + | + | ** | ++ | ++ |
| 299 | ***** | + | + | + | ** | ++ | ++ |
| 300 | ***** | + | ++ | + | * | +++ | ++ |
| 301 | ***** | + | + | + | ** | ++ | ++ |
| 302 | ***** | + | + | + | ** | +++ | ++ |
| 303 | ***** | + | + | + | ** | NT | NT |
| 304 | ***** | + | + | + | ** | +++ | ++ |
| 305 | ***** | + | + | + | * | NT | NT |
| 306 | ***** | + | + | + | *** | +++ | ++ |
| 307 | ***** | + | + | + | ** | +++ | ++ |
| 308 | ***** | + | + | + | ** | ++ | ++ |
| 309 | ***** | + | + | + | * | +++ | ++++ |
| 310 | **** | ++ | ++ | + | * | NT | NT |
| 315 | ***** | ++ | ++ | + | ** | +++ | +++ |
| 316 | ***** | ++ | ++ | ++ | ** | ++++ | ++++ |
| 317 | *** | +++ | ++ | ++ | * | NT | NT |
| 318 | **** | ++ | +++ | ++ | * | NT | NT |
| 319 | ***** | + | + | + | *** | ++ | ++ |
| 320 | ***** | ++ | + | + | ** | +++ | +++ |
| 321 | ***** | ++ | + | ++ | ** | +++ | +++ |
| 322 | ***** | ++ | ++ | + | ** | NT | NT |
| 323 | ***** | + | + | + | ** | +++ | +++ |
| 324 | ***** | + | + | + | ** | ++ | ++ |
| 325 | ***** | + | + | + | *** | NT | NT |
| 326 | ***** | + | + | + | * | +++ | +++ |
| 327 | ***** | + | + | + | ** | ++ | +++ |
| 328 | ***** | + | + | + | * | NT | NT |
| 329 | ***** | + | + | + | *** | ++ | ++ |
| 330 | ***** | + | + | + | *** | +++ | +++ |
| 331 | ***** | ++ | + | + | ** | ++ | +++ |
| 332 | ***** | ++ | ++ | + | ** | +++ | +++ |
| 333 | **** | ++ | ++ | ++ | * | +++++ | +++++ |
| 334 | *** | +++ | +++ | ++ | * | +++++ | +++++ |
| 335 | *** | ++ | +++ | ++ | * | +++++ | +++++ |
| 336 | *** | ++ | +++ | ++ | * | +++++ | +++++ |
| 337 | *** | ++ | ++ | ++ | * | NT | NT |
| 338 | *** | +++ | ++ | ++ | * | NT | NT |
| 339 | *** | ++ | ++ | ++ | * | NT | NT |
| 340 | * | +++++ | +++++ | +++ | NT | NT | NT |
| 341 | **** | ++ | ++ | ++ | * | ++++ | +++ |
| 342 | * | +++ | +++ | +++ | NT | NT | NT |
| 343 | * | +++++ | +++++ | +++++ | NT | NT | NT |
| 344 | ***** | ++ | ++ | + | * | +++ | ++ |
| 345 | *** | +++ | +++ | +++ | NT | NT | NT |
| 346 | ***** | + | ++ | + | * | +++ | +++ |
| 347 | ***** | ++ | ++ | ++ | * | ++++ | +++ |
| 348 | ***** | + | + | + | ** | ++ | ++ |
| 349 | ***** | + | ++ | + | * | +++ | ++ |
| 350 | ***** | + | ++ | + | ** | +++ | ++ |
| 351 | *** | ++ | ++ | ++ | NT | NT | NT |
| 352 | ***** | + | + | + | *** | NT | NT |
| 353 | ***** | + | + | + | *** | ++ | + |
| 354 | ***** | + | + | + | *** | + | + |
| 355 | ***** | + | + | + | *** | + | + |
| 356 | ***** | + | + | + | *** | + | ++ |
| 357 | ***** | + | + | + | *** | + | + |
| 358 | ***** | + | + | + | ** | ++ | ++ |

“*****” means IC₅₀ < 5 nM;
“****” means 5 nM ≤ IC₅₀ ≤ 20 nM;
“***” means 20 nM < IC₅₀ ≤ 50 nM;
“**” means 50 nM < IC₅₀ ≤ 100 nM;
“*” means IC₅₀ > 100 nM;
“+++++” means IC₅₀ > 10,000 nM;
“++++” means 5,000 nM < IC₅₀ ≤ 10,000 nM;
“+++” means 1,000 nM < IC₅₀ ≤ 5,000 nM;
“++” means 100 nM ≤ IC₅₀ ≤ 1,000 nM;
“+” means IC₅₀ < 100 nM

What is claimed is:

1. A compound of Formula (I-1) or (I-2): 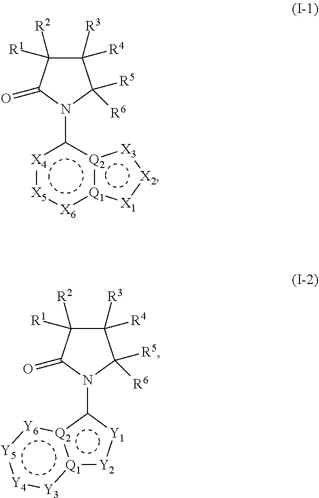
or a pharmaceutically acceptable salt thereof, wherein:
Q₁ and Q₂ each independently represent C or N;
X₁ is N, N—R^{X1}, or C—R^{X1};
X₂ is S, N—R^{X2} or C—R^{X2};
X₃ is N, O, S or C—R^{X3}, provided when X₂ is S, then X₃ cannot be S or O;
X₄ is N or C—R^{X4};
X₅ is C—R^{X5};
X₆ is N or C—R^{X6};
Y₁ is N or C—R^{Y1};
Y₂ is N—R^{Y2} or C—R^{Y2};
Y₃ is N or C—R^{Y3};
Y₄ is N or C—R^{Y4};
Y₅ is C—R^{Y5};
Y₆ is C—R^{Y6};
R^{X1}, R^{X3}, R^{X4} and R^{X6} are each independently selected from H, halo, —CN, —NR^{1a}R^{1b}, —OR^{1c}, C₁₋₄ alkyl and C₁₋₄ haloalkyl;
R^{X2} and R^{X5} are each independently selected from H, halo, CN, —NR^{1a}R^{1b}, —OR^{1c}, C₁₋₆ alkyl, C₃₋₈ cycloalkyl and R^{S}, wherein C₁₋₆ alkyl and C₃₋₈ cycloalkyl represented by R^{X2} and R^{X5} are each optionally substituted with one or more R⁸;
R^{Y1}, R^{Y3}, R^{Y4} and R^{Y6} are each independently selected from H, halo, —CN, —NR^{1a}R^{1b}, —OR^{1c}, C₁₋₄ alkyl and C₁₋₄ haloalkyl;
R^{Y2} and R^{Y5} are each independently selected from H, halo, —CN, —NR^{1a}R^{1b}, —OR^{1c}, C₁₋₆ alkyl, C₃₋₈ cycloalkyl and R^{S}, wherein C₁₋₆ alkyl and C₃₋₈ cycloalkyl represented by R^{Y2} and R^{Y5} are each optionally substituted with one or more R⁸;
R^{S} is independently selected from C₆₋₁₀ aryl, 4 to 7 membered monocyclic heterocyclyl and 5 to 10 membered heteroaryl, wherein the C₆₋₁₀ aryl, 4 to 7 membered monocyclic heterocyclyl and 5 to 10 membered heteroaryl represented by R^{S} are each optionally substituted with one or more R⁷;
R¹, R², R³, R⁴, R⁵ and R⁶, are each independently selected from H, halo, —CN, —NR^{1a}R^{1b}, —OR^{1c}, C₁₋₆ alkyl, C₃₋₈ cycloalkyl, C₆₋₁₀ aryl, 4 to 10 membered heterocycloalkyl and 5 to 10 membered heteroaryl, wherein the C₁₋₆ alkyl, C₃₋₈ cycloalkyl, C₆₋₁₀ aryl, 4 to 10 membered heterocycloalkyl and 5 to 10 membered heteroaryl represented by R¹, R², R³, R⁴, R⁵ and R⁶ are each optionally substituted with one or more substituents independently selected from halo, —CN, —NR^{1a}R^{1b}, —OR^{1c}, C₁₋₆ alkyl and C₃₋₈ cycloalkyl;
R⁷ is independently selected from halo, —CN, —NR^{1a}R^{1b}, —NR^{1a}C(O)R^{1d}, —OR^{1c}, —C(O)OR^{1c}, —C(O)NR^{1a}R^{1b}, —SO₂R^{1c}, C₁₋₆ alkyl, C₁₋₆ haloalkyl, C₃₋₆ cycloalkyl, C₆₋₁₀ aryl, 4 to 7 membered monocyclic heterocycloalkyl, 7 to 10 membered bicyclic heterocycloalkyl, and 5 to 6 membered heteroaryl; wherein the C₁₋₆ alkyl, C₁₋₆ haloalkyl, C₃₋₆ cycloalkyl, C₆₋₁₀ aryl, 4 to 7 membered monocyclic heterocycloalkyl, 7 to 10 membered bicyclic heterocycloalkyl, and 5 to 6 membered heteroaryl represented by R⁷ are each optionally substituted with one or more R^{7a};
or alternatively two of R⁷, taken together with their intervening atoms, for a 3- to 7-membered monocyclic heterocyclyl that is optionally substituted by one or two substituents independently selected from C₁₋₆ alkyl and oxo (═O);
R^{7a}, for each occurrence, is independently selected from halo, —CN, C₁₋₄ alkyl, C₁₋₄ haloalkyl, C₃₋₆ cycloalkyl, —NR^{1a}R^{1b}, —OR^{1c} and 4 to 6 membered monocyclic heterocycloalkyl, wherein the C₁₋₄ alkyl, C₃₋₆ cycloalkyl and 4 to 6 membered monocyclic heterocycloalkyl are each optionally substituted with one or more substituents independently selected from C₁₋₃alkyl, —OR^{1c}, CN and halo;
R⁸, for each occurrence, is independently halo, —CN, —NR^{1a}R^{1b}, —OR^{1c}, —C(O)OR^{1c}, C₁₋₆ alkyl, C₁₋₆ haloalkyl, C₃₋₆ cycloalkyl, C₆₋₁₀ aryl, 4 to 7 membered monocyclic heterocycloalkyl, or 5 to 6 membered heteroaryl; wherein the C₁₋₆ alkyl, C₁₋₆ haloalkyl, C₃₋₆ cycloalkyl, C₆₋₁₀ aryl, 4 to 7 membered monocyclic heterocycloalkyl, and 5 to 6 membered heteroaryl represented by R¹ are each optionally substituted with one or more substituents independently selected from halo, C₁₋₄ alkyl, C₁₋₄haloalkyl, C₃₋₆ cycloalkyl, —NR^{1a}R^{1b}, —OR^{1c} and 4 to 6 membered monocyclic heterocycloalkyl;
R^{1a} and R^{1b} are each independently H or C₁₋₄ alkyl;
R^{1c} is H, C₁₋₄ alkyl or C₁₋₄ haloalkyl;
R^{1d} is C₁₋₄ alkyl or —OR^{1e};
R^{1e} is C₁₋₄ alkyl, and
wherein at least one of R^{X1}, R^{X2}, R^{X3}, R^{X4}, R^{X5} and R^{X6} is not H, and at least one of R^{Y1}, R^{Y2}, R^{Y3}, R^{Y4}, R^{Y5} and R^{Y6} is not H.

2. The compound of claim 1, or a pharmaceutically acceptable salt thereof, wherein:
(i) Q₁ is C, and Q₂ is C;
(ii) Q₁ is N, and Q₂ is C; or
(iii) Q₁ is C, and Q₂ is N.

3. The compound of claim 1 or claim 2, or a pharmaceutically acceptable salt thereof, wherein the compound is represented by Formula (I-1), and wherein:
(i) X₁ is C—R^{X1}, X₂ is C—R^{X2}; X₄ is N or C—R^{X4}, X₅ is C—R^{X5}, X₆ is N, and X₃ is selected from N, S, O and C—R^{X3}; or
(ii) X₁ is N, X₂ is C—R^{X2}, X₄ is N or C—R^{X4}, X₅ is C—R^{X5}, X₆ is C—R^{Y6}, and X₃ is selected from N, S and C—R^{X3}.

4. The compound of claim 3, or a pharmaceutically acceptable salt thereof, wherein one of R^{X2} and R^{X5} is R^{S}, and the other one is H.

5. The compound of claim 1 or claim 2, or a pharmaceutically acceptable salt thereof, wherein the compound is represented by Formula (I-2), and wherein:
(i) Y₁ is N, Y₂ is N—R^{Y2}, Y₃ is N, Y₄ is C—R^{Y4}, Y₅ is C—R^{Y5}, and Y₆ is C—R^{Y6};
(ii) Y₁ is C—R^{Y1}, Y₂ is N—R^{Y2}, Y₃ is N, Y₄ is C—R^{Y4}, Y₅ is C—R^{Y5}, and Y₆ is C—R^{Y6};
(iii) Y₁ is C—R^{Y1}, Y₂ is C—R^{Y2}, Y₃ is N, Y₄ is C—R^{Y4}, Y₅ is C—R^{Y5}, and Y₆ is C—R^{Y6}; or
(iv) Y₁ is N, Y₂ is N—R^{Y2}, Y₃ is C—R^{Y3}, Y₄ is N, Y₅ is C—R^{Y5}, and Y₆ is C—R^{Y6}.

6. The compound of claim 5, or a pharmaceutically acceptable salt thereof, wherein one of R^{Y2} and R^{Y5} is R^{S} and the other one is H.

7. The compound of claim 1, wherein the compound is represented by one of the following formulae: 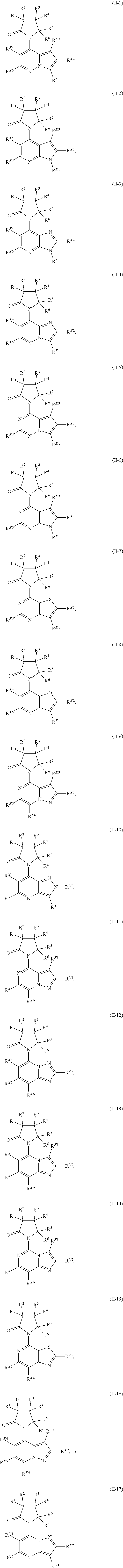
or a pharmaceutically acceptable salt thereof.

8. The compound of any one of claims 1-4 and 7, or a pharmaceutically acceptable salt thereof, wherein R^{X2} is 4- to 7-membered monocyclic heterocyclyl or 5- to 10-membered heteroaryl, wherein the 4- to 7-membered monocyclic heterocyclyl and 5- to 10-membered heteroaryl represented by R^{X2} are each optionally substituted with 1 or 2 R⁷; and R^{X5}, if present, is H.

9. The compound of any one of claims 1-4 and 7, or a pharmaceutically acceptable salt thereof, wherein R^{X5} is 4- to 7-membered monocyclic heterocyclyl or 5 to 10 membered heteroaryl, wherein the 4- to 7-membered monocyclic heterocyclyl and 5 to 10 membered heteroaryl represented by R^{X5} are each optionally substituted with 1 or 2 R⁷; and R^{X2}, if present, is H.

10. The compound of claim 1, wherein the compound is represented by one of the following formulae: 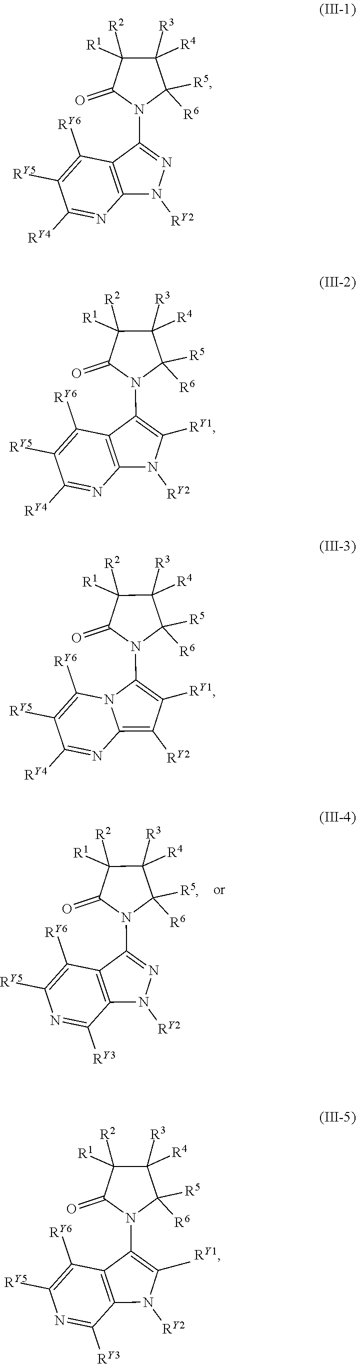
or a pharmaceutically acceptable salt thereof.

11. The compound of any one of claims 1, 2, 5, 6 and 10, or a pharmaceutically acceptable salt thereof, wherein R^{Y2} is R^{S}; and R^{S} is 4- to 7-membered monocyclic heterocyclyl or 5- to 10-membered heteroaryl, wherein the 4- to 7-membered monocyclic heterocyclyl and 5- to 10-membered heteroaryl represented by R^{S} are each optionally substituted with 1 or 2 R⁷; and R^{Y5} is H.

12. The compound of any one of claims 1, 2, 5, 6 and 10, or a pharmaceutically acceptable salt thereof, wherein R^{Y5} is R^{S}; and R^{S} is 4- to 7-membered monocyclic heterocyclyl or 5 to 10 membered heteroaryl, wherein the 4- to 7-membered monocyclic heterocyclyl and 5 to 10 membered heteroaryl represented by R^{S} are each optionally substituted with 1 or 2 R⁷; and R^{Y2} is H.

13. The compound of any one of claims 8, 9, 11 and 12, or a pharmaceutically acceptable salt thereof, wherein R^{S} is selected from pyrazine, pyridazine, pyridine, pyridin-2(1H)-one, pyrazole, pyrazolopyridine, pyrimidine, pyrrolopyridine, isoxazole, imidazole, imidazopyridine, indazole, thiazole, triazole, thiazolopyridine and triazolopyridine, each of which is optionally substituted with 1 or 2 of R⁷.

14. The compound of any one of claims 8, 9, 11 and 12, or a pharmaceutically acceptable salt thereof, wherein R^{S} is selected from: 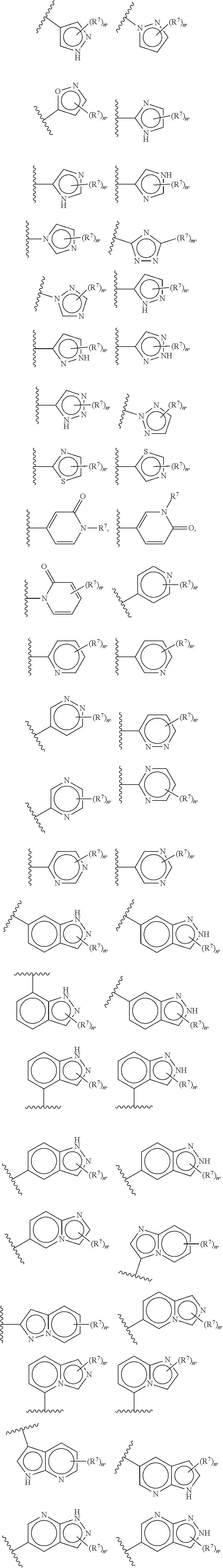 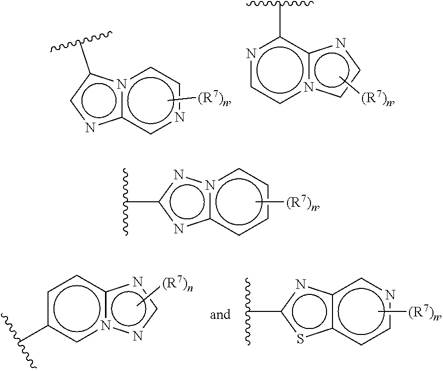
wherein: 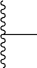
represents a bond to X₂, X₅, Y₂ or Y₅;
n represents 0, 1 or 2; and
m represents 0 or 1.

15. The compound of any one of claims 8, 9 and 11-14, or a pharmaceutically acceptable salt thereof, wherein
R⁷ is independently selected from halo, —CN, —NR^{1a}R^{1b}, —NR^{1a}C(═O)R^{1d}, —OR^{1c}, —C(O)NR^{1a}R^{1b}, —SO₂R^{1c}, C₁₋₆ alkyl, C₁₋₆ haloalkyl, C₃₋₆ cycloalkyl, 4 to 7 membered monocyclic heterocycloalkyl, 7 to 8 membered bicyclic heterocycloalkyl, and 5 to 6 membered heteroaryl; wherein the C₁₋₆ alkyl, C₁₋₆ haloalkyl, C₃₋₆ cycloalkyl, 4 to 7 membered monocyclic heterocycloalkyl, 7 to 8 membered bicyclic heterocycloalkyl, and 5 to 6 membered heteroaryl represented by R⁷ are each optionally substituted with one to three R^{7a};
or alternatively two of R⁷, taken together with their intervening atoms, for a 3 to 7 membered monocyclic heterocyclyl that is optionally substituted by one or two substituents independently selected from C₁₋₆ alkyl and =0;
R^{7a}, for each occurrence, is independently selected from C₁₋₃alkyl, halo, —CN, —OR^{1c}, C₃₋₆cycloalkyl, and 4 to 6 membered monocyclic heterocycloalkyl, wherein the C₁₋₃alkyl, C₃₋₆cycloalkyl and 4 to 6 membered monocyclic heterocycloalkyl represented by R^{7a} are each optionally substituted with one or two substituents independently selected from C₁₋₃alkyl, —OH and CN;
R^{1a} and R^{1b} are each independently H or C₁₋₄ alkyl;
R^{1c} is H or C₁₋₄ alkyl or C₁₋₄ haloalkyl;
R^{1d} is C₁₋₄ alkyl or —OR^{1c}; and
R^{1c} is C₁₋₄ alkyl.

16. The compound of any one of claims 8, 9 and 11-14, or a pharmaceutically acceptable salt thereof, wherein R⁷ is independently selected from Cl, F, —CN, —OH, —OCH₃, —O—CHF₂, —CH₃, —CD₃, —CH₂CH₃, —CH(CH₃)₂, —CHF₂, —CH₂CHF₂, —CF₃, —CH₂—OH, —CH(OH)—CH₃, —CH₂OCH₃, —CH₂CH₂OCH₂CH₃, —CH₂—CN, —N(CH₃)₂, —C(O)—NH—CH₃, —C(O)—N(CH₃)₂, —NH—C(O)—CH₃, —N(CH₃)—C(O)—O—C(CH₃)₃, —SO₂—CH₃, 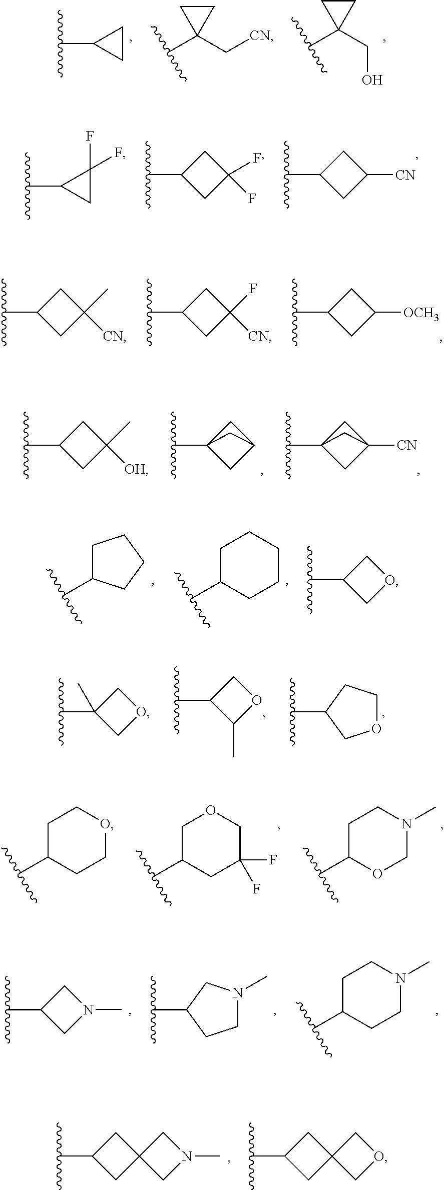 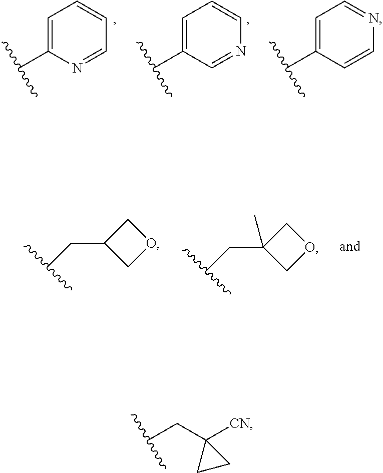
wherein 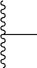
represents a bond to R^{S}.

17. The compound of any one of claims 8, 9 and 11-14, or a pharmaceutically acceptable salt thereof, wherein two of R⁷, taken together with their intervening atoms, for a 3- to 7-membered monocyclic heterocyclyl that is optionally substituted by one or two substituents independently selected from C₁₋₆ alkyl and ═O;

18. The compound of claim 17, or a pharmaceutically acceptable salt thereof, wherein R^{S} is selected from: 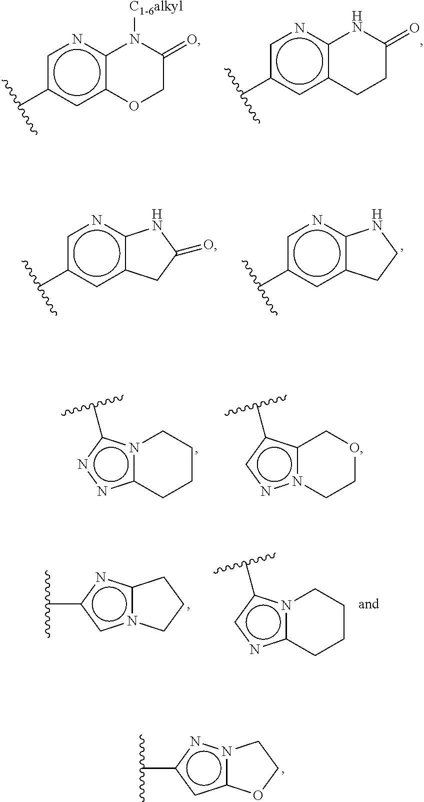
wherein 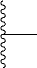
represents a bond to X₂, X₅, Y₂ or Y₅.

19. The compound of any one of claims 1-18, or a pharmaceutically acceptable salt thereof, wherein:
R¹, R², R³, R⁴, R⁵ and R⁶ are each independently selected from H, —CN, C₁₋₆ alkyl, C₁₋₆ haloalkyl, C₃₋₆ cycloalkyl and 4 to 6 membered heterocycloalkyl, wherein the C₁₋₆ alkyl is optionally substituted with —CN or —OR^{1f}; and
R^{1f} is C₁₋₄ alkyl.

20. The compound of claim 19, or a pharmaceutically acceptable salt thereof, wherein R¹, R², R³, R⁴, R⁵ and R⁶ are each independently selected from H, CN, —CH₃, —CH₂CH₃, cyclopropyl, cyclobutyl, —CHF₂, —CH₂—O—CH₃, CH₂—CN and

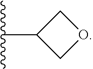 [SMILES: *C1COC1]

21. The compound of any one of claims 1-18, or a pharmaceutically acceptable salt thereof, wherein:
R¹ and R² are each independently selected from H, CN, C₁₋₆ alkyl, C₁₋₆ haloalkyl, C₃₋₆ cycloalkyl and 4 to 10 membered heterocycloalkyl, wherein the C₁₋₆ alkyl is optionally substituted with —CN or —OR^{1f};
R³ and R⁴ are each independently selected from H, C₁₋₆ alkyl and C₁₋₆ haloalkyl;
R⁵ and R⁶ are each independently H or C₁₋₆ alkyl; and
R^{1f} is C₁₋₄ alkyl.

22. The compound of claim 21, or a pharmaceutically acceptable salt thereof, wherein:
R¹ and R² are each independently selected from H, CN, —CH₃, —CH₂CH₃, —CHF₂, cyclopropyl, cyclobutyl, —CH₂—O—CH₃, —CH₂—CN and: 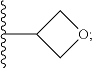 [SMILES: *C1COC1]
R³ and R⁴ are each independently selected from H, —CH₃, —CH₂CH₃ and —CHF₂; and
R⁵ and R⁶ are each independently selected from H, —CH₃ and —CH₂CH₃.

23. The compound of any one of claims 1-22, or a pharmaceutically acceptable salt thereof, wherein:
R^{X1}, R^{X3}, R^{X4} and R^{X6} are each independently selected from H, halo, and C₁₋₄ alkyl; and
R^{Y1}, R^{Y3}, R^{Y4} and R^{Y6} are each independently selected from H, halo, and C₁₋₄ alkyl.

24. The compound of claim 23, or a pharmaceutically acceptable salt thereof, wherein:
R^{X1}, R^{X3}, R^{X4} and R^{X6} are each independently selected from H, Cl, F, and CH₃; and
R^{Y1}, R^{Y3}, R^{Y4} and R^{Y6} are each independently selected from H, Cl, F, and CH₃.

25. The compound of claim 23, or a pharmaceutically acceptable salt thereof, wherein:
R^{X1}, R^{X3}, R^{X4} and R^{X6} are H; and
R^{Y1}, R^{Y3}, R^{Y4} and R^{Y6} are H.

26. The compound of claim 1, wherein the compound is represented by the following formula: 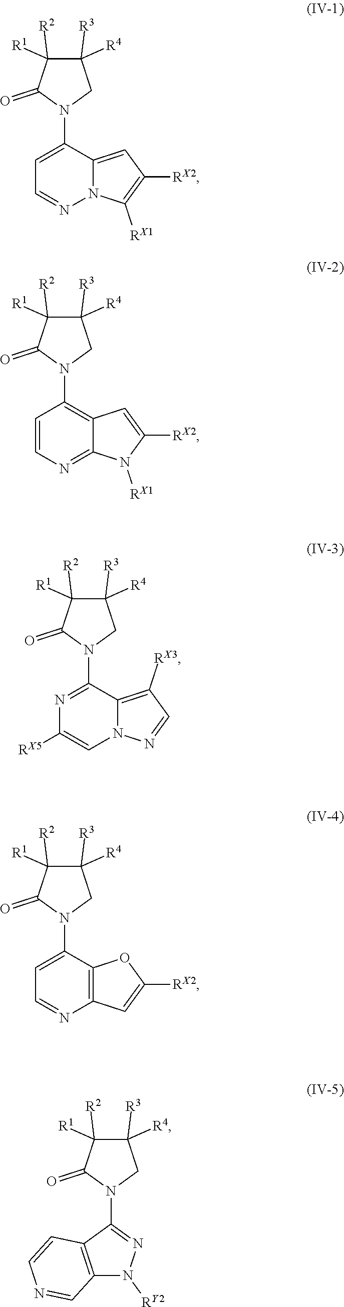
or a pharmaceutically acceptable salt thereof, wherein:
R^{X1} is H or halo;
R^{X2} is 5-membered heteroaryl optionally substituted with 1 or 2 R⁷;
R^{X3} is H or halo;
R^{X5} is 5- or 6-membered heteroaryl optionally substituted with 1 or 2 R⁷;
R¹² is 5-membered heteroaryl optionally substituted with 1 or 2 R⁷;
R¹ and R² are each independently —CN or C₃₋₆ cycloalkyl; and
R³ and R⁴ are each independently H or C₁₋₃ alkyl.

27. The compound of claim 26, or a pharmaceutically acceptable salt thereof, wherein the 5- or 6-membered heteroaryl represented by R^{X2}, R^{X5} or R^{Y2} is pyrazole or pyridine, each of which is optionally substituted with 1 R⁷.

28. The compound of claim 26, or a pharmaceutically acceptable salt thereof, wherein the 5- or 6-membered heteroaryl represented by R^{X2}, R^{X5} or R^{Y2} is represented by one of the following formula:

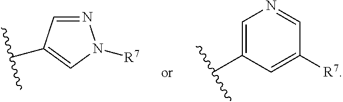

29. The compound of claim 27 or 28, wherein R⁷ is halo, C₁₋₄ alkyl, C₁₋₄ haloalkyl or 4 to 6 membered monocyclic heterocycloalkyl containing 1 or 2 heteroatoms independently selected from oxygen and nitrogen.

30. The compound of claim 29, or a pharmaceutically acceptable salt thereof, wherein R⁷ is F, —CH₃, —CHF₂, —CH₂CH₃ or

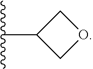 [SMILES: *C1COC1]

31. The compound of any one of claims 26-30, or a pharmaceutically acceptable salt thereof, wherein R¹ is CN, R² is C₃₋₆cycloalkyl, R³ is H and R⁴ is C₁₋₃alkyl.

32. The compound of claim 31, or a pharmaceutically acceptable salt thereof, wherein R¹ is CN, R² is cyclopropyl, R³ is H and R⁴ is —CH₃.

33. A pharmaceutical composition comprising a compound according to any one of claims 1-32, or a pharmaceutically acceptable salt thereof, and a pharmaceutically acceptable carrier.

34. A method of inhibiting tyrosine kinase 2 (TYK2) activity in a subject in need thereof comprising administering to the subject an effective amount of a compound according to any one of claims 1-32 or a pharmaceutically acceptable salt thereof or a pharmaceutical composition according to claim 33.

35. A method of treating a disease or disorder responsive to inhibition of tyrosine kinase 2 (TYK2) in a subject comprising administering to the subject an effective amount of a compound according to any one of claims 1-32 or a pharmaceutically acceptable salt thereof or a pharmaceutical composition according to claim 33.

36. The method of claim 35, wherein the disease or disorder is inflammation, autoimmune disease, neuroinflammation, arthritis, rheumatoid arthritis, spondyloarthropathies, systemic lupus erythematous, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis and sunburn.
